(19) United States
(12) Patent Application Publication
(10) Pub. No.: US 2010/0152160 A1
(43) Pub. Date: Jun. 17, 2010

(54) NOVEL BENZODIOXANE AND BENZOXAZINE DERIVATIVES USEFUL AS CC CHEMOKINE RECEPTOR LIGANDS

(76) Inventors: Uttam KHAMRAI, Kolkata (IN); Matthew Ronsheim, Port Jefferson, NY (US); Sumit Kumar Karak, Kolkata (IN)

Correspondence Address: Forest Laboratories, Inc.;Attn: Charles S. Ryan, 48 Mall Drive, Commack, NY, 11725, US

(21) Appl. No.: 12/635,907

(22) Filed: Dec. 11, 2009

Related U.S. Application Data
(60) Provisional application No. 61/121,937, filed on Dec. 12, 2008.

(51) Int. Cl. C07D 413/06; C07D 405/06; A61K 31/496; A61K 31/551; C07D 413/02; A61K 31/538; A61K 31/553; A61P 29/00; A61P 37/00; A61P 3/00; A61P 3/10

(52) U.S. Cl. 514/210.21; 544/377; 514/254.11; 514/218; 540/575; 544/105; 514/230.5; 514/211.15; 540/544

(57) ABSTRACT

The present invention relates to benzodioxane and benzoxazine derivatives that act as ligands for CC chemokine receptors, such as CCR1. The invention also relates to methods of preparing the compounds, compositions containing the compounds, and to methods of treatment using the compounds.

### FIELD OF THE INVENTION

The present invention relates to benzodioxane and benzoxazine derivatives that act as ligands for CC chemokine receptors, such as CCR1. The invention also relates to methods of preparing the compounds, compositions containing the compounds, and to methods of treatment using the compounds.

### BACKGROUND OF THE INVENTION

The selective accumulation and activation of leukocytes in inflamed tissues contributes to the pathogenesis of inflammatory and autoimmune diseases. Chemokines and their receptors, which belong to a family of seven transmembrane G-protein coupled receptors are involved in the selective accumulation and activation of leukocytes in inflamed tissues, and in the pathogenesis of inflammatory and autoimmune diseases. One such receptor is CCR1 which is a receptor for CC chemokines such as RANTES (regulated on activation normal Tcell expressed), MIP-1α (macrophage inflammatory protein) MPIF-1/CKβ8 and Leukotactin chemokine, among others.

The receptor CCR1 and its chemokine ligands represent significant therapeutic targets (see, e.g., Saeki, et al., Current Pharmaceutical Design, 9, 1201-1208, 2003) since they have been implicated in, for example, rheumatoid arthritis, transplant rejection (see, e.g., DeVries, et al., Semin. Immunol., 11(2), 95-104, 1999), and multiple sclerosis (see, e.g., Fischer, et al., J. Neuroimmunol., 110(1-2), 195-208, 2000, Izikson, et al., J Exp. Med., 192(7), 1075-1080, 2000, and Rottman, et al., Eur. J. Immunol., 30(8), 2372-2377, 2000). In vivo studies on mice indicate that CCR1-mediated leukocyte recruitment is important for interstitial inflammation in the kidney and that CCR1 blockade late in renal disease can halt disease progression and improve renal function (see, e.g., NAME, J. Am. Soc. Nephrol., 15, 1504-1513, 2004). Further, an animal model of neutrophil recruitment in response to MIP-1α demonstrates the positive biological and pharmacodynamic activity of CCR1 antagonists (see, e.g., U.S. 2005/0288319).

There is therefore an ongoing need to develop new compounds that can be used in the treatment of diseases mediated by CCR1 signaling.

### SUMMARY OF THE INVENTION

The present invention relates to benzodioxane and benzoxazine derivatives that act as ligands for CC chemokine receptors, such as CCR1. The invention also relates to methods of preparing the compounds, compositions containing the compounds, and to methods of treatment using the compounds.

### DETAILED DESCRIPTION OF THE INVENTION

In one aspect, the present invention includes compounds having the chemical structure:

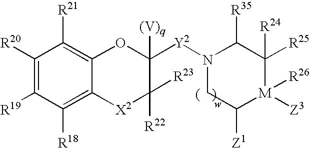

wherein

X² is O or —NR¹⁷, where R¹⁷ is hydrogen, alkyl, acyl, heteroaryl, —C(O)—CF₃, cyano, alkylsulfinyl, alkylsulfonyl, amido, —C(O)-aminoalkyl, -alkyleneamino, -alkylene-OH, -alkylene-C(O)NR^{e}R^{f}, or -alkylene-NR^{e}R^{f}, where R^{e} and R^{f} are each, independently, hydrogen or alkyl;

Y² is —C(O)—, —CH₂C(O)—, —CH₂CH₂— or —CH₂—;

M is selected from the group consisting of —C—, —N—, and —O—;

Z¹ is selected from the group consisting of hydrogen, aryl, heteroaryl, heterocycle, arylalkyl, heteroarylalkyl or heterocyclealkyl;

Z³ is —C(O)-aryl, —C(O)-heteoraryl, —C(O)-arylalkyl, or —C(O)-heteroarylalkyl, arylalkyl or heteroarylalkyl;

R¹⁸, R¹⁹, R²⁰ and a R²¹ are each, independently, hydrogen, halogen, hydroxy, cyano, nitro, amino, alkylamino, dialkylamino, carboxyl, alkyl, alkenyl, alkynyl, cycloalkyl, cycloalkylalkyl, aryl, arylalkyl, heteroaryl, heteroarylamino, heteroarylalkyl, heterocycle, heterocyclealkyl, aroyl, acyl, alkoxy, aryloxy, cycloalkyloxy, cycloalkylalkyloxy, arylalkyloxy, heteroarylalkyloxy, alkythio, arylthio, alkylsulfinyl, alkylsulfonyl, alkoxycarbonyl, aryloxycarbonyl, heteroaryloxycarbonyl —O-alkylene-CO₂H, —O-alkylene-C(O)NR^{x}R^{y} or NR^{x}C(O)-alkyl, in which R^{x} and R^{y} are each, independently, hydrogen or alkyl;

R²² and R²³ are each hydrogen, or R²² and R²³, together with the carbon atom to which they are attached, form a —C(O)— group;

R²⁴ and a R²⁵ are each, independently, hydrogen, alkyl or arylalkyl;

V is alkyl;

w is 0, 1 or 2;

q is 0 or 1;

R²⁶ K is hydrogen, hydroxyl, —CO₂Et, cyano, heteroaryl, -alkylene-OH, or -alkylene-NH₂; wherein

when R²⁶ is hydrogen, then X² is -NR¹⁷— and Z³ is —C(O)-aryl, —C(O)-heteoraryl, —C(O)-arylalkyl, or —C(O)-heteroarylalkyl; and

when R²⁶ is hydroxyl, —CO₂Et, cyano, heteroaryl, -alkylene-OH, or -alkylene-NH₂, then Z³ is arylalkyl or heteroarylalkyl;

R³⁵ is hydrogen or alkyl;

provided, however, that when R²⁶ is present, then M is —C—;

provided, however, that when Z³ is present, then M is —C— or —N—;

provided, however, than when M is —O—, then Z¹ is arylalkyl;

provided, however, than when w is 1, X² is —O—, and M is —N—, then Z³ is optionally substituted 4-fluorobenzyl,

wherein, when present, any aryl, heteroaryl or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, and combinations thereof;

and pharmaceutically acceptable salts or solvates or N-oxides thereof, or solvates of pharmaceutically acceptable salts thereof, or pharmaceutically acceptable salts or solvates of N-oxides thereof, prodrugs and isomers thereof,

with the proviso that said compound is not:

[4-[(2-chloro-4-fluorophenypmethyl]-1-piperazinyl]-(2,3-dihydro-1,4-benzodioxin-2-yl)-methanone,

1-[2,3-dihydro-2-[[4-(1-naphthalenylmethyl)-1-piperazinyl]carbonyl]-4H-1,4-benzoxazin-4-yl]-ethanone,

1-[2,3-dihydro-2-[[4-[[4-(trifluoromethyl)phenyl]methyl]-1-piperazinyl]carbonyl]4H-1,4-benzoxazin-4-yl]-ethanone,

[4-(ethylsulfonyl)-3,4-dihydro-2H-1,4-benzoxazin-2-yl][4-(phenylmethyl)-1-piperazinyl]-methanone,

2-[2-oxo-2-[4-(phenylmethyl)-1-piperazinyl]ethyl]-2H-1,4,-benzoxazin-3(4H)-one, or

1-2[[hexahydro-4-[(4-methoxyphenyl)methyl]-1H-1,4-diazepin-1-yl]carbonyl]-2,3-dihydro-4H-1,4-benzoxazin-4-yl]-ethanone,

1-[2-[(4-benzoyl-1-piperidinyl)carbonyl]-2,3-dihydro-4H-1,4-benzoxazin-4-yl]-ethanone,

1-[2-[(4-(4-hydroxy)benzoyl-1-piperidinyl)carbonyl]-2,3-dihydro-4H-1,4-benzoxazin-4-yl]-ethanone,

1-[2-(2,3-dihydro-1,4-benzodioxin-2-yl)ethyl]-4-(4-methylphenyl)-4-piperidinol, or a pharmaceutically acceptable salt thereof.

In another aspect, the present invention includes compounds of formula I:

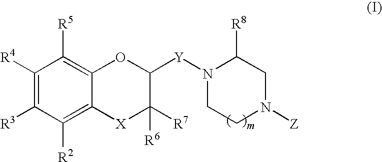

wherein

X is —O— or —NR¹—, where R¹ is hydrogen, alkyl, acyl, alkylsulfinyl, alkylsufonyl, amido or —C(O)-aminoalkyl;

Y is —C(O)— or —CH₂C(O)—;

R², R³, R⁴ and R⁵ are each, independently, hydrogen, halogen, hydroxy, cyano, nitro, amino, alkylamino, dialkylamino, carboxyl, alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, cycloalkyloxy, cycloalkylalkyloxy, arylalkyloxy, heteroarylalkyloxy, alkythio, arylthio, alkylsulfinyl, alkylsulfonyl, alkoxycarbonyl, aryloxycarbonyl or heteroaryloxycarbonyl;

R⁶ and R⁷ are each hydrogen, or R⁶ and R⁷, together with the carbon atom to which they are attached, form a —C(O)— group;

R⁸ is hydrogen or alkyl;

Z is arylalkyl; and

m is 1 or 2;

wherein, when present, any aryl, heteroaryl or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof;

with the provisos that:

when m is 1 and X is —O—, then Z is optionally substituted 4-fluorobenzyl,

and said compound is not:

[4-[(2-chloro-4-fluorophenyl)methyl]-1-piperazinyl](2,3-dihydro-1,4-benzodioxin-2-yl)-methanone,

1-[2,3-dihydro-2-[[4-(1-naphthalenylmethyl)-1-piperazinyl]carbonyl]-4H-1,4-benzoxazin-4-yl]-ethanone,

1-[2,3-dihydro-2-[[4-[[4-(trifluoromethyl)phenyl]methyl]-1-piperazinyl]carbonyl]4H-1,4-benzoxazin-4-yl]-ethanone,

[4-(ethylsulfonyl)-3,4-dihydro-2H-1,4-benzoxazin-2-yl][4-(phenylmethyl)-1-piperazinyl]-methanone,

2-[2-oxo-2-[4-(phenylmethyl)-1-piperazinyl]ethyl]-2H-1,4,-benzoxazin-3(4H)-one, or

1-2[[hexahydro-4-[(4-methoxyphenyl)methyl]-1H-1,4-diazepin-1-yl]carbonyl]-2,3 -dihydro-4H-1,4-benzoxazin-4-yl]-ethanone,

or a pharmaceutically acceptable salt thereof.

In additional embodiments, one of R²-R⁵ is halogen (e.g., Cl). For example, one of R²-R⁵ is halogen (e.g., Cl) and the others of R²-R⁵ are hydrogen. For further example, R², R⁴ and R⁵ are hydrogen and R³ is halogen (e.g., Cl). In another embodiment, two of R²-R⁵ are halogen (e.g., Cl). For example, R² and R⁵ are hydrogen and R³ and R⁴ are halogen (e.g., Cl).

In certain embodiments, when X is —NR¹—, then R², R⁴ and R⁵ are hydrogen and R³ is halogen (e.g., Cl).

In one embodiment, when R⁶ and R⁷, together with the carbon atom to which they are attached, form a —C(O)— group, then Y is —C(O)—.

In further embodiments, when X is —O—, then Y is —C(O)—.

In additional embodiments, X is —NR¹—, where R¹ is hydrogen, alkyl (e.g., methyl), acyl (e.g., —C(O)CH₃), alkylsufonyl (e.g., —SO₂CH₃), amido (—C(O)NH₂) or —C(O)-aminoalkyl (e.g., —C(O)CH₂NHMe, —C(O)CH₂NMe₂).

In certain embodiments, R⁸ is alkyl (e.g., methyl). In other embodiments, R⁸ is hydrogen.

In further embodiments, Z is 4-halo-substituted arylalkyl (e.g., 4-halo-substituted benzyl, such as 4-fluorobenzyl). In further embodiments, Z is 4-halo-substituted arylalkyl (e.g., 4-halo-substituted benzyl, such as 4-fluorobenzyl) that is not additionally substituted by halogen. In further embodiments, Z is 4-halo-substituted arylalkyl (e.g., 4-halo-substituted benzyl, such as 4-fluorobenzyl) that is not additionally substituted by alkoxy (e.g., methoxy). In further embodiments, Z is 4-halo-substituted arylalkyl (e.g., 4-halo-substituted benzyl, such as 4-fluorobenzyl) that is not additionally substituted by cyano. In further embodiments, Z is 4-halo-substituted arylalkyl (e.g., 4-halo-substituted benzyl, such as 4-fluorobenzyl) that is not additionally substituted by halogen, alkoxy or cyano.

In additional embodiments, Z is 4-halo-substituted arylalkyl (e.g., 4-halo-substituted benzyl, such as 4-fluorobenzyl) that is not further substituted. In exemplary embodiments, Z is unsubstituted 4-fluoro-arylalkyl, such as unsubstituted 4-fluorobenzyl.

In certain embodiments, the present invention includes compounds of formula (I) wherein

X is O or —NR¹, where R¹ is hydrogen, alkyl, acyl, alkylsufonyl, amido or —C(O)-aminoalkyl;

Y is —C(O)— or —CH₂C(O)—;

R², R³, R⁴ and R⁵ are each, independently, hydrogen or halogen;

R⁶ and R⁷ are each hydrogen, or R⁶ and R⁷, together with the carbon atom to which they are attached, form a —C(O)— group;

R⁸ is hydrogen or alkyl;

m is 1 or 2; and

Z is arylalkyl;

with the proviso that when m is 1 and X is O, then Z is 4-fluorobenzyl.

In certain embodiments, the compound of formula I is selected from:

(6-Chloro-2,3-dihydro-benzo[1,4]dioxin-2-yl)-[(R)-4-(4-fluoro-benzyl)-2-methyl-piperazin-1-yl]-methanone,

(2,3-Dihydro-benzo[1,4]dioxin-2-yl)-[(R)-4-(4-fluoro-benzyl)-2-methyl-piperazin-1-yl]-methanone,

(2,3-Dihydro-benzo[1,4]dioxin-2-yl)-[4-(4-fluoro-benzyl)-piperazin-1-yl]-methanone,

(6-Chloro-2,3-dihydro-benzo[1,4]dioxin-2-yl)-[4-(4-fluoro-benzyl)-piperazin-1-yl]-methanone,

(6-Chloro-2,3-dihydro-benzo[1,4]dioxin-2-yl)-[4-(4-fluoro-benzyl)-[1,4]diazepan-1yl]-methanone,

(6,7-Dichloro-2,3-dihydro-benzo[1,4]dioxin-2-yl)-[(R)-4-(4-fluoro-benzyl)-2-methyl-piperazin-1-yl]-methanone,

(6,7-Dichloro-2,3-dihydro-benzo[1,4]dioxin-2-yl)-[4-(4-fluoro-benzyl)-[1,4]diazepan-1-yl]-methanone,

(6-Chloro-3,4-dihydro-2H-benzo[1,4]oxazin-2-yl)-[(R)-4-(4-fluoro-benzyl)-2-methyl-piperazin-1-yl]-methanone,

(6-Chloro-3,4-d,ihydro-2H-benzo[1,4]oxazin-2-yl)-[4-(4-fluoro-benzyl)-piperazin-1-yl]-methanone,

(6-Chloro-3,4-dihydro-2H-benzo[1,4]oxazin-2-yl)-[4-(4-fluoro-benzyl)-[1,4]diazepan-1-yl]-methanone,

6-Chloro-2-[4-(4-fluoro-benzyl)-[1,4]diazepane-1-carbonyl]-2,3-dihydro-benzo[1,4]oxazine-4-carboxylic acid amide,

(6-Chloro-4-methyl-3,4-dihydro-2H-benzo[1,4]oxazin-2-yl)-[(R)-4-(4-fluoro-benzyl)-2-methyl-piperazin-1-yl]-methanone,

(6-Chloro-4-methyl-3,4-dihydro-2H-benzo[1,4]oxazin-2-yl)-[4-(4-fluoro-benzyl)-[1,4]diazepan-1-yl]-methanone,

6-Chloro-2-[(R)-4-(4-fluoro-benzyl)-2-methyl-piperazine-1-carbonyl]-4H-benzo[1,4]oxazin-3-one,

1-{6-Chloro-2-[4-(4-fluoro-benzyl)-piperazine-1-carbonyl]-2,3-dihydro-benzo[1,4]oxazin-4-yl}-ethanone,

(6-Chloro-4-methanesulfonyl-3,4-dihydro-2H-benzo[1,4]oxazin-2-yl)-[4-(4-fluoro-benzyl)-piperazin-1-yl]-methanone,

1-{6-Chloro-2-[4-(4-fluoro-benzyl)-piperazine-1-carbonyl]-2,3-dihydro-benzo[1,4]oxazin-4-yl}-2-dimethylamino-ethanone,

(6-Chloro-4-methyl-3,4-dihydro-2H-benzo[1,4]oxazin-2-yl)-[4-(4-fluoro-benzyl)-piperazin-1-yl]-methanone,

1-{6-Chloro-2-[4-(4-fluoro-benzyl)-piperazine-1-carbonyl]-2,3-dihydro-benzo[1,4]oxazin-4-yl}-2-methylamino-ethanone,

2-(6-Chloro-3,4-dihydro-2H-benzo[1,4]oxazin-2-yl)-1-[(R)-4-(4-fluoro-benzyl)-2-methyl-piperazin-1-yl]-ethanone,

2-(6-Chloro-3,4-dihydro-2H-benzo[1,4]oxazin-2-yl)-1-[4-(4-fluoro-benzyl)-[1,4]diazepan-1-yl]-ethanone, and

(6-Chloro-7-methoxy-3,4-dihydro-2H-benzo[1,4]oxazin-2-yl)-[(R)-4-(4-fluoro-benzyl)-2-methyl-piperazin-1-yl]-methanone,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In additional embodiments, the compound of formula I is selected from:

(6-Chloro-2,3-dihydro-benzo[1,4]dioxin-2-yl)-[4-(4-fluoro-benzyl)-2-methyl-piperazin-1-yl]-methasone,

(2,3-Dihydro-benzo[1,4]dioxin-2-yl)-[4-(4-fluoro-benzyl)-2-methyl-piperazin-1-yl]-methanone,

(6,7-Dichloro-2,3-dihydro-benzo[1,4]dioxin-2-yl)-[4-(4-fluoro-benzyl)-2-methyl-piperazin-1-yl]-methanone,

(6-Chloro-4-methyl-3,4-dihydro-2H-benzo[1,4]oxazin-2-yl)-[4-(4-fluoro-benzyl)-2-methyl-piperazin-1-yl]-methanone, and

(6-Chloro-7-methoxy-4-methyl-3,4-dihydro-2H-benzo[1,4]oxazin-2-yl)-[(R)-4-(4-fluoro-benzyl)-2-methyl-piperazin-1-yl]-methanone,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In another aspect, the present invention includes compounds of formula II:

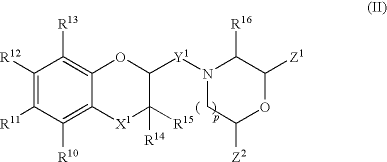

wherein

X¹ is O or —NR⁹, where R⁹ is hydrogen, alkyl, acyl, alkylsulfinyl, alkylsulfonyl, amido or —C(O)-aminoalkyl;

Y¹ is —C(O)—, —CH₂C(O)—, —CH₂— or —CH₂CH₂—;

R¹⁰, R¹¹, R¹² and R¹³ are each, independently, hydrogen, halogen, hydroxy, cyano, nitro, amino, alkylamino, dialkylamino, carboxyl, alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, cycloalkyloxy, cycloalkylalkyloxy, arylalkyloxy, heteroarylalkyloxy, alkythio, arylthio, alkylsulfinyl, alkylsulfonyl, alkoxycarbonyl, aryloxycarbonyl or heteroaryloxycarbonyl;

R¹⁴ and R¹⁵ are each hydrogen, or R¹⁴ and R¹⁵, together with the carbon atom to which they are attached, form a —C(O)— group;

R¹⁶ is hydrogen or alkyl;

p is 1 or 2; and

Z¹ are Z² are each, independently, hydrogen, aryl, heteroaryl, heterocycle, arylalkyl, heteroarylalkyl or heterocyclealkyl; with the proviso that at least one of Z¹ or Z² is other than hydrogen;

wherein, when present, any aryl, heteroaryl or heterocycle may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof.

In additional embodiments, one of R¹⁰—R¹³ is halogen (e.g., Cl). For example, one of R¹⁰—R¹³ is halogen (e.g., Cl) and the others of R¹⁰—R¹³ are hydrogen. For further example, R¹⁰, R¹² and R¹³ are hydrogen and R¹¹ is halogen (e.g., Cl). In another embodiment, two of R¹⁰—R¹³ are halogen (e.g., Cl).

In certain embodiments, X is —NR⁹, where R⁹ is hydrogen, alkyl, acyl, alkylsulfinyl, alkylsulfonyl, amido or —C(O)-aminoalkyl. For example X is —NH—.

In further embodiments, one of Z¹ and Z² is hydrogen and the other of Z¹ and Z² is arylalkyl (e.g., benzyl), such as as 4-halo-substituted arylalkyl (e.g., 4-fluorobenzyl). For example, Z¹ is hydrogen and Z² is arylalkyl (e.g., benzyl), such as as 4-halo-substituted arylalkyl (e.g., 4-fluorobenzyl). For further example, Z² is hydrogen and Z¹ is arylalkyl (e.g., benzyl), such as as 4-halo-substituted arylalkyl (e.g., 4-fluorobenzyl). In certain embodiments, Z¹ is hydrogen and Z² is 4-fluorobenzyl. In additional embodiments, Z² is hydrogen and Z¹ is 4-fluorobenzyl.

In certain embodiments, the present invention includes compounds of formula II

wherein

X¹ is —NR⁹, where R⁹ is hydrogen;

Y¹ is —C(O)—, —CH₂C(O)—, or —CH₂CH₂—;

R¹⁰, R¹¹, R¹² and R¹³ are each, independently, hydrogen or halogen;

R¹⁴ and R¹⁵ are each hydrogen;

R¹⁶ is hydrogen;

p is 2; and

Z¹ are Z² are each, independently, hydrogen or arylalkyl; with the proviso that at least one of Z¹ or Z² is arylalkyl.

In certain embodiments, the compound of formula II is selected from:

(6-Chloro-3,4-dihydro-2H-benzo[1,4]oxazin-2-yl)-[7-(4-fluoro-benzyl)-[1,4]oxazepan-4-yl]-methanone,

2-(6-Chloro-3,4-dihydro-2H-benzo[1,4]oxazin-2-yl)-1-[7-(4-fluoro-benzyl)-[1,4]oxazepan-4-yl]-ethanone,

2-(6-Chloro-3,4-dihydro-2H-benzo[1,4]oxazin-2-yl)-1-[2-(4-fluoro-benzyl)-[1,4]oxazepan-4-yl]-ethanone, and

6-Chloro-2-{2-[2-(4-fluoro-benzyl)-[1,4]oxazepan-4-yl]-ethyl}-3,4-dihydro-2H-benzo[1,4]oxazine,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In a further aspect, the present invention includes compounds of formula III:

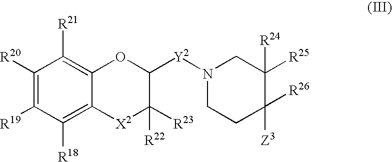

wherein

X² is O or —NR¹⁷, where R¹⁷ is hydrogen, alkyl, acyl, alkylsulfinyl, alkylsulfonyl, amido, —C(O)-aminoalkyl or -alkylene-C(O)NR^{e}R^{f}, where R^{e} and R^{f} are each, independently, hydrogen or alkyl;

Y² is —C(O)—, —CH₂C(O)—, —CH₂CH₂— or —CH₂—;

R¹⁸, R¹⁹, R²⁰ and R²¹ are each, independently, hydrogen, halogen, hydroxy, cyano, nitro, amino, alkylamino, dialkylamino, carboxyl, alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, cycloalkyloxy, cycloalkylalkyloxy, arylalkyloxy, heteroarylalkyloxy, alkythio, arylthio, alkylsulfinyl, alkylsulfonyl, alkoxycarbonyl, aryloxycarbonyl, heteroaryloxycarbonyl —O-alkylene-CO₂H, —O-alkylene-C(O)NR^{x}R^{y} or NR^{x}(O)-alkyl, in which R^{x} and R^{y} are each, independently, hydrogen or alkyl;

R²² and R²³ are each hydrogen, or R²² and R²³, together with the carbon atom to which they are attached, form a —C(O)-group;

R²⁴ and R²⁵ are each, independently, hydrogen or alkyl; and

R²⁶ is hydrogen, hydroxyl, cyano or -alkylene-NH₂; wherein

when R²⁶ is hydrogen, then X² is —NR¹⁷— and Z³ is —C(O)-aryl, -C(O)-heteoraryl, —C(O)-arylalkyl, or —C(O)-heteroarylalkyl; and

when R²⁶ is hydroxyl, cyano or -alkylene-NH₂, then Z³ is arylalkyl or heteroarylalkyl;

wherein, when present, any aryl, heteroaryl or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof,

with the proviso that said compound is not:

1-[2-[(4-benzoyl-1-piperidinyl)carbonyl]-2,3-dihydro-4H-1,4-benzoxazin-4-yl]-ethanone,

1-[2-[(4-(4-hydroxy)benzoyl-1-piperidinyl)carbonyl]-2,3-dihydro-4H-1,4-benzoxazin-4-yl]-ethanone,

1-[2-(2,3-dihydro-1,4-benzodioxin-2-yl)ethyl]-4-(4-methylphenyl)-4-piperidinol, or a pharmaceutically acceptable salt thereof.

In additional embodiments, R¹⁸—R²¹ are each hydrogen. In further embodiments one of R¹⁸—R²¹ is halogen (e.g., Cl). For example, one of R¹⁸—R²¹ is halogen (e.g., Cl) and the others of R¹⁸—R²¹ are hydrogen. In another embodiment, two of R¹⁸—R²¹ are halogen (e.g., Cl). In further embodiments, one of R¹⁸—R²¹ is halogen (e.g., Cl) and another of R¹⁸—R²¹ is alkoxy, hydroxyl, dialkylamino, —O-alkylene-CO₂H, —O-alkylene-C(O)NR^{x}R^{y} or NR^{x}C(O)-alkyl, in which R^{x} and R^{y} are each independently hydrogen or alkyl.

For example, R¹⁸, R²⁰ and R²¹ are hydrogen and R¹⁹ is halogen (e.g., Cl). For further example, R¹⁹ is halogen (e.g., Cl) and R²⁰ is hydrogen, alkoxy (e.g., methoxy, ethoxy), dialkylamino (e.g., -dimethylamino), hydroxyl, —O-alkylene-CO₂H, —O-alkylene-C(O)NR^{x}R^{y} or NR^{x}C(O)-alkyl, in which R^{x} and R^{y} are each independently hydrogen or alkyl. For example, R¹⁹ is halogen (e.g., Cl), R²⁰ is hydrogen, alkoxy, hydroxyl, dialkylamino, —O-alkylene-CO₂H, —O-alkylene-C(O)NR^{x}R^{y} or NR^{x}C(O)-alkyl, in which R^{x} and R^{y} are each independently hydrogen or alkyl, and R¹⁸ and R²⁰ are hydrogen. For example R¹⁸ and R²⁰ are hydrogen, R¹⁹ is halogen (e.g., Cl) and R²⁰ is hydrogen, methoxy, ethoxy, hydroxyl, —NMe₂, —OCH₂CO₂H, —OCH₂C(O)NH₂, —OCH₂C(O)NMe₂ or —NHC(O)CH₃.

In certain embodiments, X² is —NR¹⁷, where R¹⁷ is hydrogen, alkyl, acyl, alkylsulfinyl, alkylsulfonyl, amido, —C(O)-aminoalkyl or -alkylene-C(O)NR^{e}R^{f}. For example R¹⁷ is hydrogen, alkyl, acyl, alkylsulfonyl, —C(O)-aminoalkyl or -alkylene-C(O)NR^{e}R^{f}. In certain embodiments, R^{e} and R^{f} are both hydrogen. For further example, X² is —NH— or —CH₂C(O)NH₂. For example, X² is —NH—.

In further embodiments, R²² and R²³ are hydrogen.

In additional embodiments, R²⁶ is hydroxyl, cyano or -alkylene-NH₂.

In further embodiments, R²⁴ and R²⁵ are alkyl. For example, R²⁴ and R²⁵ are methyl.

In certain embodiments, R²⁴ and R²⁵ are alkyl and R²⁶ is hydroxyl. For example, R²⁴ and R²⁵ are methyl and R²⁶ is hydroxyl.

In further embodiments, R²⁴ and R²⁵ are hydrogen and R²⁶ is cyano or -alkylene-NH₂. For example, R²⁴ and R²⁵ are hydrogen and R²⁶ is cyano.

In additional embodiments, Z³ is halo-substituted arylalkyl. In exemplary embodiments, Z³ is benzyl. For further example, Z³ is halo-substituted benzyl, such as 4-halo-substitued benzyl (e.g., 4-fluorobenzyl, 4-chlorobenzyl).

In further embodiments, Z³ is substituted arylalkyl. In exemplary embodiments, the benzyl hydrogens are substituted with for example, but not limited to, hydroxyl, fluoro or difluoro groups.

In certain embodiments, the present invention includes compounds of formula III wherein

X² is —NR¹⁷, where R¹⁷ is hydrogen, alkyl, acyl, alkylsulfonyl, —C(O)-aminoalky or or -alkylene-C(O)NR^{e}R^{f}, where R^{e} and R^{f} are each, independently, hydrogen or alkyl;

Y² is —C(O)—, —CH₂C(O)—, —CH₂CH₂— or —CH₂—;

R¹⁸, R¹⁹, R²⁰ and R²¹ are each, independently, hydrogen, halogen, hydroxy, alkoxy, dialkylamino, —O-alkylene-CO₂H or —O-alkylene-C(O)NR^{x}R^{y}, in which R^{x} and R^{y} are each, independently, hydrogen or alkyl;

R²² and R²³ are hydrogen;

R²⁴ and R²⁵ are each, independently, hydrogen or alkyl; and

R²⁶ is hydrogen, hydroxyl, cyano or -alkylene-NH₂; wherein

when R²⁶ is hydrogen, then Z³ is —C(O)-aryl; and

when R²⁶ is hydroxyl, cyano or -alkylene-NH₂, then Z³ is arylalkyl.

In certain embodiments, the present invention includes compounds of formula III wherein

X² is —NR¹⁷, where R¹⁷ is hydrogen, alkyl, acyl, alkylsulfonyl, —C(O)-aminoalkyl, dialkylamino or -alkylene-C(O)NR^{e}R^{f} where R^{e} and R^{f} are each, independently, hydrogen or alkyl;

Y² is —C(O)—,

R¹⁸, R¹⁹, R²⁰ and R²¹ are each, independently, hydrogen, halogen, or alkoxy;

R²² and R²³ are hydrogen;

R²⁴ and R²⁵ are hydrogen;

R²⁶ is cyano; and

Z³ is arylalkyl.

For example, X² is —NH—, Y² is —C(O)—, R¹⁸ and R²¹ are each hydrogen, R¹⁹ and R²⁰ are each, independently, halogen (e.g., Cl) or alkoxy (e.g., methoxy), R²² and R²³ are hydrogen, R²⁴ and R²⁵ are hydrogen, R²⁶ is cyano and Z³ is arylalkyl.

In additional embodiments, the compound of formula III is represented by subformulas IIIa-IIIb:

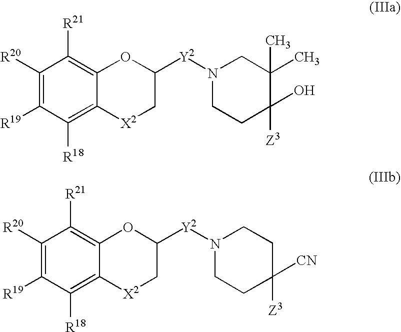

For example, in subformula IIIa, X² is NR¹⁷, Y² is —C(O)—, —CH₂C(O)—, —CH₂CH₂— or —CH₂—, R¹⁹ is halogen and R¹⁸, R²⁰ and R²¹ are hydrogen. In additional embodiments, R¹⁷ is hydrogen.

For example, in subformula IIIb, X² is NR¹⁷, Y² is —C(O)—, R¹⁹ is halogen and R¹⁸ and R²¹ are hydrogen, R²⁰ is hydrogen, alkoxy (e.g., methoxy), hydroxyl, O-alkylene-CO₂H (e.g., —OCH₂CO₂H)— or —O-alkylene-C(O)NR^{x}R (e.g., —OCH₂C(O)NH₂, —OCH2C(O)NMe₂).

In certain embodiments, the compound of formula III is selected from:

(6-Chloro-3,4-dihydro-2H-benzo[1,4]oxazin-2-yl)-[4-(4-fluoro-benzoyl)-piperidin-1-yl]-methanone,

1-(6-Chloro-3,4-dihydro-2H-benzo[1,4]oxazine-2-carbonyl)-4-(4-fluoro-benzyl)-piperidine-4-carbonitrile,

1-(6-Chloro-3,4-dihydro-2H-benzo[1,4]oxazin-2-ylmethyl)-4-(4-fluoro-benzyl)-piperidin-4-yl)-methylamine,

1-(6-Chloro-7-methoxy-3,4-dihydro-2H-benzo[1,4]oxazine-2-carbonyl)-4-(4-fluoro-benzyl)-piperidine-4-carbonitrile,

4-(4-Chloro-benzyl)-1-(6-chloro-3,4-dihydro-2H-1-benzo[1,4]oxazine-2-carbonyl)-piperidine-4-carbonitrile,

[4-(4-Chloro-benzyl)-4-hydroxy-3,3-dimethyl-piperidin-1-yl]-(6-chloro-3,4-dihydro-2H-benzo[1,4]oxazin-2-yl)-methanone,

4-(4-Chloro-benzyl)-1-(6-chloro-3,4-dihydro-2H-benzo[1,4]oxazin-2-ylmethyl)-3,3-dimethyl-piperidin-4-ol,

1-(6-Chloro-7-methoxy-4-methyl-3,4-dihydro-2H-benzo[1,4]oxazine-2-carbonyl)-4-(4-fluoro-benzyl)-piperidine-4-carbonitrile,

1-(6-Chloro-7-hydroxy-3,4-dihydro-2H-benzo[1,4]oxazine-2-carbonyl)-4-(4-fluoro-benzyl)-piperidine-4-carbonitrile,

2-{6-Chloro-2-[4-cyano-4-(4-fluoro-benzyl)-piperidine-1-carbonyl]-3,4-dihydro-2H-benzo[1,4]oxazin-7-yloxy}-acetic acid,

2-{6-Chloro-2-[4-cyano-4-(4-fluoro-benzyl)-piperidine-1-carbonyl]-3,4-dihydro-2H-benzo[1,4]oxazin-7-yloxy}-acetamide,

2-{6-Chloro-2-[4-cyano-4-(4-fluoro-benzyl)-piperidine-1-carbonyl]-3,4-dihydro-2H-benzo[1,4]oxazin-7-yloxy}-N,N-dimethyl-acetamide,

1-(6-Chloro-7-ethoxy-3,4-dihydro-2H-benzo[1,4]oxazine-2-carbonyl)-4-(4-fluoro-benzyl)-piperidine-4-carbonitrile,

1-[6-Chloro-4-(2-dimethylamino-acetyl)-7-methoxy-3,4-dihydro-2H-benzo[1,4]oxazine-2-carbonyl]-4-(4-fluoro-benzyl)-piperidine-4-carbonitrile,

1-(4-Acetyl-6-chloro-7-methoxy-3,4-dihydro-2H-benzo[1,4]oxazine-2-carbonyl)-4-(4-fluoro-benzyl)-piperidine-4-carbonitrile,

1-[4-(4-Chloro-benzyl)-4-hydroxy-3,3-dimethyl-piperidin-1-yl]-2-(6-chloro-3,4-dihydro-2H-benzo[1,4]oxazin-2-yl)-ethanone,

4-(4-Chloro-benzyl)-1-[2-(6-chloro-3,4-dihydro-2H-benzo[1,4]oxazin-2-yl)-ethyl]-3,3-dimethyl-piperidin-4-ol,

1-[2-(6-Chloro-3,4-dihydro-2H-benzo[1,4]oxazin-2-yl)-ethyl]-4-(4-fluoro-benzyl)-3,3-dimethyl-piperidin-4-ol,

1-(6-Chloro-4-methanesulfonyl-7-methoxy-3,4-dihydro-2H-benzo[1,4]oxazine-2-carbonyl)-4-(4-fluoro-benzyl)-piperidine-4-carbonitrile,

2-{6-Chloro-2-[4-cyano-4-(4-fluoro-benzyl)-piperidine-1-carbonyl]-7-methoxy-2,3-dihydro-benzo[1,4]oxazin-4-yl}-acetamide,

1-(6-Chloro-7-dimethylamino-3,4-dihydro-2H-benzo[1,4]oxazine-2-carbonyl)-4-(4-fluoro-benzyl)-piperidine-4-carbonitrile,

N-{6-Chloro-2-[4-cyano-4-(4-fluoro-benzyl)-piperidine-1-carbonyl]-3,4-dihydro-2H-benzo[1,4]oxazin-7-yl}-acetamide,

4-(4-Fluoro-benzyl)-1-(7-methoxy-3,4-dihydro-2H-benzo[1,4]oxazine-2-carbonyl)-piperidine-4-carbonitrile,

6-Chloro-2-[4-cyano-4-(4-fluoro-benzyl)-piperidine-l-carbonyl]-7-methoxy-2,3-dihydro-benzo[1,4]oxazine-4-carboxylic acid amide,

1-(7-Bromo-6-chloro-3,4-dihydro-2H-benzo[1,4]oxazine-2-carbonyl)-4-(4-fluoro-benzyl)-piperidine-4-carbonitrile,

1-(6-Chloro-8-dimethylamino-3,4-dihydro-2H-benzo[1,4]oxazine-2-carbonyl)-4-(4-fluoro-benzyl)-piperidine-4-carbonitrile,

1-(6,7-Dichloro-3,4-dihydro-2H-benzo[1,4]oxazine-2-carbonyl)-4-(4-fluoro-benzyl)-piperidine-4-carbonitrile,

1-(6-Chloro-7-isopropoxy-3,4-dihydro-2H-benzo[1,4]oxazine-2-carbonyl)-4-(4-fluoro-benzyl)-piperidine-4-carbonitrile,

1-(4-Acetyl-6-chloro-7-dimethylamino-3,4-dihydro-2H-benzo[1,4]oxazine-2-carbonyl)-4-(4-fluoro-benzyl)-piperidine-4-carbonitrile,

2-{6-Chloro-2-[4-cyano-4-(4-fluoro-benzyl)-piperidine-1-carbonyl]-7-dimethylamino-2,3-dihydro-benzo[1,4]oxazin-4-yl}-acetamide,

1-(6-Chloro-7-dimethylamino-4-methanesulfonyl-3,4-dihydro-2H-benzo[1,4]oxazine-2-carbonyl)-4-(4-fluoro-benzyl)-piperidine-4-carbonitrile,

2-{6-Chloro-2-[4-cyano-4-(4-fluoro-benzyl)-piperidine-1-carbonyl]-7-isopropoxy-2,3-dihydro-benzo[1,4]oxazin-4-yl}-acetamide,

2-{6-Chloro-2-[4-cyano-4-(4-fluoro-benzyl)-piperidine-1-carbonyl]-7-ethoxy-2,3-dihydro-benzo[1,4]oxazin-4-yl}-acetamide,

1-(6-Chloro-7-ethoxy-4-methanesulfonyl-3,4-dihydro-2H-benzo[1,4]oxazine-2-carbonyl)-4-(4-fluoro-benzyl)-piperidine-4-carbonitrile,

1-(6-Chloro-7-pyrrolidin-1-yl-3,4-dihydro-2H-benzo[1,4]oxazine-2-carbonyl)-4-(4-fluoro-benzyl)-piperidine-4-carbonitrile,

1-(7-Azetidin-1-yl-6-chloro-3,4-dihydro-2H-benzo[1,4]oxazine-2-carbonyl)-4-(4-fluoro-benzyl)-piperidine-4-carbonitrile,

1-(6-Chloro-7-difluoromethoxy-3,4-dihydro-2H-benzo[1,4]oxazine-2-carbonyl)-4-(4-fluoro-benzyl)-piperidine-4-carbonitrile,

1-(7-Chloro-6-methoxy-3,4-dihydro-2H-benzo[1,4]oxazine-2-carbonyl)-4-(4-fluoro-benzyl)-piperidine-4-carbonitrile,

1-(6-Chloro-7-diethylamino-3,4-dihydro-2H-benzo[1,4]oxazine-2-carbonyl)-4-(4-fluoro-benzyl)-piperidine-4-carbonitrile,

1-(6-Chloro-7-morpholin-4-yl-3,4-dihydro-2H-benzo[1,4]oxazine-2-carbonyl)-4-(4-fluoro-benzyl)-piperidine-4-carbonitrile,

(6-Chloro-7-methoxy-3,4-dihydro-2H-benzo[1,4]oxazin-2-yl)-[4-(4-fluoro-phenoxy)-piperidin-1-yl]-methanone,

1-[6-Chloro-4-(2-hydroxy-ethyl)-7-methoxy-3,4-dihydro-2H-benzo[1,4]oxazine-2-carbonyl]-4-(4-fluoro-benzyl)-piperidine-4-carbonitrile,

1-[6-Chloro-7-dimethylamino-4-(2-hydroxy-ethyl)-3,4-dihydro-2H-benzo[1,4]oxazine-2-carbonyl]-4-(4-fluoro-benzyl)-piperidine-4-carbonitrile,

6-Chloro-2-[4-cyano-4-(4-fluoro-benzyl)-piperidine-l-carbonyl]-7-dimethylamino-2,3-dihydro-benzo[1,4]oxazine-4-carbonitrile,

1-(6-Chloro-7-dimethylamino-3,4-dihydro-2H-benzo[1,4]oxazine-2-carbonyl)-4-(6-fluoro-pyridin-3-ylmethyl)-piperidine-4-carbonitrile,

1-(6-Chloro-7-methylamino-3,4-dihydro-2H-benzo[1,4]oxazine-2-carbonyl)-4-(4-fluoro-benzyl)-piperidine-4-carbonitrile,

2-{6-Chloro-2-[4-cyano-4-(4-fluoro-benzyl)-piperidine-1-carbonyl]-7-methoxy-2,3-dihydro-benzo[1,4]oxazin-4-yl}-N-methyl-acetamide,

1-(6-Chloro-7-methoxy-3,4-dihydro-2H-benzo[1,4]oxazine-2-carbonyl)-4-(6-fluoro-pyridin-3-ylmethyl)-piperidine-4-carbonitrile,

6-Chloro-2-[4-cyano-4-(4-fluoro-benzyl)-piperidine-1-carbonyl]-7-methoxy-2,3-dihydro-benzo[1,4]oxazine-4-carbonitrile,

1-(6-Chloro-7-methoxy-3,4-dihydro-2H-benzo[1,4]oxazine-2-carbonyl)-4-(4-fluoro-benzyl)-piperidine-4-carboxylic acid ethyl ester,

1-(6-Chloro-7-dimethylamino-3,4-dihydro-2H-benzo[1,4]oxazine-2-carbonyl)-4-(4-fluoro-benzyl)-piperidine-4-carboxylic acid ethyl ester,

1-(6-Chloro-7-methoxy-3,4-dihydro-2H-benzo[1,4]oxazine-2-carbonyl)-4-pyridin-4-ylmethyl-piperidine-4-carbonitrile,

1-(6-Chloro-7-dimethylamino-3,4-dihydro-2H-benzo[1,4]oxazine-2-carbonyl)-4-pyridin-4-ylmethyl-piperidine-4-carbonitrile,

(6-Chloro-7-methoxy-3,4-dihydro-2H-benzo[1,4]oxazin-2-yl)-[4-(4-fluoro-benzyl)-4-hydroxymethyl-piperidin-1-yl]-methanone,

(6-Chloro-7-dimethylamino-3,4-dihydro-2H-benzo[1,4]oxazin-2-yl)-[4-(4-fluoro-benzyl)-4-hydroxymethyl-piperidin-1-yl]-methanone,

1-(6-Chloro-7-dimethylamino-3,4-dihydro-2H-benzo[1,4]oxazine-2-carbonyl)-4-[(4-fluoro-phenyl)-hydroxy-methyl]-piperidine-4-carbonitrile,

1-[6-Chloro-7-dimethylamino-4-(2-dimethylamino-ethyl)-3,4-dihydro-2H-benzo[1,4]oxazine-2-carbonyl]-4-(4-fluoro-benzyl)-piperidine-4-carbonitrile,

1-[6-Chloro-4-(2-dimethylamino-ethyl)-7-methoxy-3,4-dihydro-2H-benzo[1,4]oxazine-2-carbonyl]-4-(4-fluoro-benzyl)-piperidine-4-carbonitrile,

(6-Chloro-7-methoxy-3,4-dihydro-2H-benzo[1,4]oxazin-2-yl)-[4-(4-fluoro-benzyl)-4-imidazol-1-ylmethyl-piperidin-1-yl]-methanone,

1-(6-Chloro-7-ethyl-3,4-dihydro-2H-benzo[1,4]oxazine-2-carbonyl)-4-(4-fluoro-benzyl)-piperidine-4-carbonitrile,

1-(6-Chloro-7-methoxy-3,4-dihydro-2H-benzo[1,4]oxazine-2-carbonyl)-4-[(4-fluoro-phenyl)-hydroxy-methyl]-piperidine-4-carbonitrile,

1-(6-Chloro-7-dimethylamino-3,4-dihydro-2H-benzo[1,4]oxazine-2-carbonyl)-3-(4-fluoro-benzyl)-pyrrolidine-3-carbonitrile,

{6-Chloro-2-[4-cyano-4-(4-fluoro-benzyl)-piperidine-1-carbonyl]-3,4-dihydro-2H-benzo[1,4]oxazin-7-yl}-methyl-cyanamide,

1-(6-Chloro-7-methoxy-3,4-dihydro-2H-benzo[1,4]oxazine-2-carbonyl)-4-[difluoro-(4-fluoro-phenyl)-methyl]-piperidine-4-carbonitrile,

1-(6-Chloro-7-dimethylamino-3,4-dihydro-2H-benzo[1,4]oxazine-2-carbonyl)-4-[difluoro-(4-fluoro-phenyl)-methyl]-piperidine-4-carbonitrile,

1-[6-Chloro-7-(1H-pyrazol-4-yl)-3,4-dihydro-2H-benzo[1,4]oxazine-2-carbonyl]-4-(4-fluoro-benzyl)-piperidine-4-carbonitrile,

6-Chloro-7-[methyl-(2H-tetrazol-5-yl)-amino]-3,4-dihydro-2H-benzo[1,4]oxazine-2-carboxylic acid [3-cyano-3-(4-fluoro-benzyl)-pentyl]-methyl-amide,

1-[6-Chloro-7-(2H-tetrazol-5-ylamino)-3,4-dihydro-2H-benzo[1,4]oxazine-2-carbonyl]-4-(4-fluoro-benzyl)-piperidine-4-carbonitrile,

1-[6-Chloro-7-dimethylamino-4-(2,2,2-trifluoro-acetyl)-3,4-dihydro-2H-benzo[1,4]oxazine-2-carbonyl]-4-(4-fluoro-benzyl)-piperidine-4-carbonitrile,

1-[6-Chloro-7-methoxy-4-(2,2,2-trifluoro-acetyl)-3,4-dihydro-2H-benzo[1,4]oxazine-2-carbonyl]-4-(4-fluoro-benzyl)-piperidine-4-carbonitrile,

1-(6-Chloro-7-methoxy-3-oxo-3,4-dihydro-2H-benzo[1,4]oxazine-2-carbonyl)-4-(4-fluoro-benzyl)-piperidine-4-carbonitrile,

1-(6-Chloro-7-imidazol-1-yl-3,4-dihydro-2H-benzo[1,4]oxazine-2-carbonyl)-4-(4-fluoro-benzyl)-piperidine-4-carbonitrile,

1-[6-Chloro-7-methoxy-4-(2H-tetrazol-5-yl)-3,4-dihydro-2H-benzo[1,4]oxazine-2-carbonyl]-4-(4-fluoro-benzyl)-piperidine-4-carbonitrile,

4-(4-Fluoro-benzyl)-1-(quinoxaline-2-carbonyl)-piperidine-4-carbonitrile,

4-(4-Fluoro-benzyl)-1-([1,2,4]triazolo[1,5-a]pyrimidine-2-carbonyl)-piperidine-4-carbonitrile,

4-(4-Fluoro-benzyl)-1-(pyrazolo[1,5-a]pyrimidine-3-carbonyl)-piperidine-4-carbonitrile

6-Chloro-2-[4-cyano-4-(4-fluoro-benzyl)-piperidine-1-carbonyl]-2,3-dihydro-benzo[1,4]oxazine-4-carbonitrile,

1-[6-Chloro-4-(2H-tetrazol-5-yl)-3,4-dihydro-2H-benzo[1,4]oxazine-2-carbonyl]-4-(4-fluoro-benzyl)-piperidine-4-carbonitrile,

1-[6-Chloro-4-(1H-[1,2,4]triazol-3-yl)-3,4-dihydro-2H-benzo[1,4]oxazine-2-carbonyl]-4-(4-fluoro-benzyl)-piperidine-4-carbonitrile,

1-(6-Chloro-7-pyrrol-1-yl-3,4-dihydro-2H-benzo[1,4]oxazine-2-carbonyl)-4-(4-fluoro-benzyl)piperidine-4-carbonitrile,

1-[6-Chloro-7-methoxy-4-(1H-[1,2,4]triazol-3-yl)-3,4-dihydro-2H-benzo[1,4]oxazine-2-carbonyl]-4-(4-fluoro-benzyl)-piperidine-4-carbonitrile,

1-[6-Chloro-8-(1H-pyrazol-4-yl)-3,4-dihydro-2H-benzo[1,4]oxazine-2-carbonyl]-4-(4-fluoro-benzyl)-piperidine-4-carbonitrile,

1-(6-Chloro-8-pyridin-4-yl-3,4-dihydro-2H-benzo[1,4]oxazine-2-carbonyl)-4-(4-fluoro-benzyl)-piperidine-4-carbonitrile,

1-(6-Chloro-8-thiophen-3-yl-3,4-dihydro-2H-benzo[1,4]oxazine-2-carbonyl)-4-(4-fluoro-benzyl)-piperidine-4-carbonitrile,

1-(6-Chloro-7-thiophen-3-yl-3,4-dihydro-2H-benzo[1,4]oxazine-2-carbonyl)-4-(4-fluoro-benzyl)-piperidine-4-carbonitrile,

1-(6-Chloro-7-pyridin-3-yl-3,4-dihydro-2H-benzo[1,4]oxazine-2-carbonyl)-4-(4-fluoro-benzyl)-piperidine-4-carbonitrile,

1-(6-Chloro-7-pyridin-4-yl-3,4-dihydro-2H-benzo[1,4]oxazine-2-carbonyl)-4-(4-fluoro-benzyl)-piperidine-4-carbonitrile,

1-[6-Chloro-7-(2-methyl-2H-pyrazol-3-yl)-3,4-dihydro-2H-benzo[1,4]oxazine-2-carbonyl]-4-(4-fluoro-benzyl)-piperidine-4-carbonitrile,

1-[6-Chloro-7-(1-methyl-1H-pyrazol-4-yl)-3,4-dihydro-2H-benzo[1,4]oxazine-2-carbonyl]-4-(4-fluoro-benzyl)-piperidine-4-carbonitrile,

1-(6-Chloro-7-dimethylamino-3,4-dihydro-2H-benzo[1,4]oxazine-2-carbonyl)-4-(4-fluoro-benzyl)-piperidine-4-carbonitrile,

1-(6-Chloro-7-pyrazol-1-yl-3,4-dihydro-2H-benzo[1,4]oxazine-2-carbonyl)-4-(4fluoro-benzyl)-piperidine-4-carbonitrile,

1-(6-Chloro-7-pyrimidin-5-yl-3,4-dihydro-2H-benzo[1,4]oxazine-2-carbonyl)-4-(4-fluoro-benzyl)-piperidine-4-carbonitrile,

1-(6-Chloro-7-hexa deuterio dimethylamino-3,4-dihydro-2H-benzo[1,4]oxazine-2-carbonyl)-4-(4-fluoro-benzyl)-piperidine-4-carbonitrile,

1-(6-Chloro-7-furan-2-yl-3,4-dihydro-2H-benzo[1,4]oxazine-2-carbonyl)-4-(4-fluoro-benzyl)-piperidine-4-carbonitrile,

1-(6-Chloro-7-furan-3-yl-3,4-dihydro-2H-benzo[1,4]oxazine-2-carbonyl)-4-(4-fluoro-benzyl)-piperidine-4-carbonitrile,

1-[6-Chloro-7-(4-methyl-imidazol-1-yl)-3,4-dihydro-2H-benzo[1,4]oxazine-2-carbonyl]-4-(4-fluoro-benzyl)-piperidine-4-carbonitrile,

1-[6-Chloro-7-(3-methyl-pyrazol-1-yl)-3,4-dihydro-2H-benzo[1,4]oxazine-2-carbonyl]-4-(4-fluoro-benzyl)-piperidine-4-carbonitrile,

1-(6-Chloro-7-[1,2,4]triazol-4-yl-3,4-dihydro-2H-benzo[1,4]oxazine-2-carbonyl)-4-(4-fluoro-benzyl)-piperidine-4-carbonitrile,

1-(6-Chloro-7-[1,2,3]triazol-1-yl-3,4-dihydro-2H-benzo[1,4]oxazine-2-carbonyl]-4-fluoro-benzyl)-piperidine-4-carbonitrile,

1-(6-Chloro-4-methyl-7-oxazol-5-yl-3,4-dihydro-2H-benzo[1,4]oxazine-2-carbonyl)-4-fluoro-benzyl)-piperidine-4-carbonitrile,

1-[6-Chloro-7-dimethylamino-4-(1H-[1,2,4]triazol-3-yl)-3,4-dihydro-2H-benzo[1,4]oxazine-2-carbonyl]-4-(4-fluoro-benzyl)-piperidine-4-carbonitrile,

1-[6-Chloro-7-(6-fluoro-pyridin-3-yl)-3,4-dihydro-2H-benzo[1,4]oxazine-2-carbonyl]-4-(4-fluoro-benzyl)-piperidine-4-carbonitrile,

1-[6-Chloro-7-(6-methoxy-pyridin-3-yl)-3,4-dihydro-2H-benzo[1,4]oxazine-2-carbonyl]-4-(4-fluoro-benzyl)-piperidine-4-carbonitrile,

1-[6-Chloro-7-(2-methoxy-pyridin-3-yl)-3,4-dihydro-2H-benzo[1,4]oxazine-2-carbonyl]-4-(4-fluoro-benzyl)-piperidine-4-carbonitrile,

1-(6-Chloro-7-hydroxymethyl-3,4-dihydro-2H-benzo[1,4]oxazine-2-carbonyl)-4-(4-fluoro-benzyl)-piperidine-4-carbonitrile,

1-(6-Chloro-2-methyl-3,4-dihydro-2H-benzo[1,4]oxazine-2-carbonyl)-4-(4-fluoro-piperidine-4-carbonitrile,

1-(6-Chloro-7-dimethylamino-2-methyl-3,4-dihydro-2H-benzo[1,4]oxazine-2-carbonyl)-4-(4-fluoro-benzyl)-piperidine-4-carbonitrile,

6-Chloro-2-[4-cyano-4-(4-fluoro-benzyl)-piperidine-1-carbonyl]-7-dimethylamino-2,3-dihydro-benzo[1,4]oxazine-4-carbonitrile,

1-[6-Chloro-7-(2-methoxy-phenyl)-3,4-dihydro-2H-benzo[1,4]oxazine-2-carbonyl]-4-(4-fluoro-benzyl)-piperidine-4-carbonitrile,

1-(6-Chloro-7-pyridin-2-yl-3,4-dihydro-2H-benzo[1,4]oxazine-2-carbonyl)-4-(4-fluoro-benzyl)-piperidine-4-carbonitrile,

1-(7-Acetyl-6-chloro-3,4-dihydro-2H-benzo[1,4]oxazine-2-carbonyl)-4-(4-fluoro-benzyl)-piperidine-4-carbonitrile,

1-(6-Chloro-7-oxazol-5-yl-3,4-dihydro-2H-benzo[1,4]oxazine-2-carbonyl)-4-(4-fluoro-benzyl)-piperidine-4-carbonitrile,

1-(4-Chloro-2-methyl-3,6,7,8-tetrahydro-9-oxa-1,3,6-triaza-cyclopenta[a]naphthalene-8-carbonyl)-4-(4-fluoro-benzyl)-piperidine-4-carbonitrile,

1-(4-Chloro-3,6,7,8-tetrahydro-9-oxa-1,3,6triazacyclopenta[a]naphthalene-8-carbonyl)-4-(4-fluoro-benzyl)-piperidine-4-carbonitrile,

6-Chloro-2-[4-cyano-4-(4-fluoro-benzyl)-piperidine-1-carbonyl]-3,4-dihydro-2H-benzo[1,4]oxazine-7-carboxylic acid methylamide,

6-Chloro-2-[4-cyano-4-(4-fluoro-benzyl)-piperidine-1-carbonyl]-3,4-dihydro-2H-benzo[1,4]oxazine-7-carbonitrile,

6-Chloro-2-[4-cyano-4-(4-fluoro-benzyl)-piperidine-1-carbonyl]-3,4-dihydro-2H-benzo[1,4]oxazine-7-carboxylic acid dimethylamide, and

1-[6-Chloro-7-(2-methyl-thiazol-4-yl)-3,4-dihydro-2H-benzo[1,4]oxazine-2-carbonyl]-4-(4-fluoro-benzyl)-piperidine-4-carbonitrile,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In another aspect, the present invention includes compounds of formula IV:

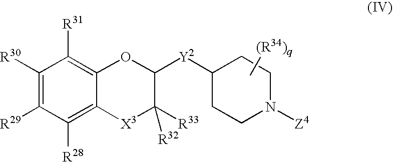

wherein

X³ is O or —NR²⁷, where R²⁷ is hydrogen, alkyl, acyl, alkylsulfinyl, alkylsulfonyl, amido or —C(O)-aminoalkyl;

Y² is —C(O)—, —CH₂C(O)—, —CH₂—, —CH₂CH₂—, —CH₂NR′, —C(O)NR^{z}, —CH₂C(O)NR^{z} or —CH₂CH₂N^{z}, where R^{z} is hydrogen or alkyl;

R²⁸, R²⁹, R³⁰ and R³¹ are each, independently, hydrogen, halogen, hydroxy, cyano, nitro, amino, alkylamino, dialkylamino, carboxyl, alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, cycloalkyloxy, cycloalkylalkyloxy, arylalkyloxy, heteroarylalkyloxy, alkythio, arylthio, alkylsulfinyl, alkylsulfonyl, alkoxycarbonyl, aryloxycarbonyl or heteroaryloxycarbonyl;

R³² and R³³ are each hydrogen, or R³² and R³³, together with the carbon atom to which they are attached, form a —C(O)— group;

R³⁴ is alkyl;

q is 0, 1, 2, 3 or 4; and

Z⁴ is arylalkyl;

wherein, when present, any aryl, heteroaryl or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof,

with the proviso that said compound is not:

2,3-dihydro-N-[1-(phenylmethyl)-4-piperidinyl]-1,4-benzodioxin-2-carboxamide,

or a pharmaceutically acceptable salt thereof.

In certain embodiments, X³ is —NR²⁷, where R²⁷ is hydrogen, alkyl, acyl, alkylsulfinyl, alkylsulfonyl, amido or —C(O)-aminoalkyl. For example, X³ is —NH—.

In additional embodiments, Y² is —C(O)NR^{z}, —CH₂C(O)NR^{z} or —CH₂CH₂NR^{z}, where R^{z} is hydrogen or alkyl. For example Y² is —C(O)NR^{z}, —CH₂C(O)NR^{z} or —CH₂CH₂NR^{z}, where R^{z} is hydrogen.

In further embodiments, q is 0, 1 or 2. For example, q is 0 or 1. For further example, q is 0.

In additional embodiments, one of R²⁸—R³¹ is halogen (e.g., Cl). For example, one of R²⁸—R³¹ is halogen (e.g., Cl) and the others of R²⁸—R³¹ are hydrogen. For further example, R²⁸, R³⁰ and R³¹ are hydrogen and R²⁹ is halogen (e.g., Cl). In another embodiment, two of R²⁸—R³¹ are halogen (e.g., Cl).

In additional embodiments, Z⁴ is halo-substituted arylalkyl. In exemplary embodiments, Z⁴ is benzyl. For further example, Z⁴ is halo-substituted benzyl, such as 4-halo-substitued benzyl (e.g., 4-fluorobenzyl, 4-chlorobenzyl).

In certain embodiments, the present invention includes compounds of formula IV

wherein
X³ is —NR²⁷, where R²⁷ is hydrogen;
Y² is —C(O)NR^{z}, —CH₂C(O)NR^{z} or —CH₂CH₂NR^{z}, where R^{z} is hydrogen;
R²⁸, R²⁹, R³⁰ and R³¹ are each, independently, hydrogen or halogen;
R³² and R³³ are each hydrogen;
q is 0; and
Z⁴ is arylalkyl.

In certain embodiments, the compound of formula IV is selected from:

6-Chloro-3,4-dihydro-2H-benzo[1,4]oxazine-2-carboxylic acid [1-(4-fluoro-benzyl)-piperidin-4-yl]-amide,

N-[1-(4-Chloro-benzyl)-piperidin-4-yl]-2-(6-chloro-3,4-dihydro-2H-benzo[1,4]oxazin-2-yl)-acetamide,

[1-(4-Chloro-benzyl)-piperidin-4-yl]-[2-(6-chloro-3,4-dihydro-2H-benzo[1,4]oxazin-2-yl)-ethyl]-amine,

6-Chloro-7-methoxy-3,4-dihydro-2H-benzo[1,4]oxazine-2-carboxylic acid [1-(4-fluoro-benzyl)-piperidin-4-yl]-amide,

6-Chloro-7-methoxy-3,4-dihydro-2H-benzo[1,4]oxazine-2-carboxylic acid [1-(4-fluoro-benzyl)-4-hydroxy-piperidin-4-ylmethyl]-amide, and

6-Chloro-7-dimethylamino-3,4-dihydro-2H-benzo[1,4]oxazine-2-carboxylic acid [1-(4-fluoro-benzyl)-4-hydroxy-piperidin-4-ylmethyl]-amide,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

As used herein the term “halogen” means F, Cl, Br, and I.

The term “alkyl” means a substituted or unsubstituted saturated hydrocarbon radical which may be straight-chain or branched-chain and may comprise about 1 to about 20 carbon atoms, for instance 1 to 12 carbon atoms, such as 1 to 8 carbon atoms, e.g., 1 to 4 carbon atoms. Suitable alkyl groups include, but are not limited to, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to, 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, methylhexyl, dimethylpentyl, ethylpentyl, ethylmethylbutyl, dimethylbutyl, and the like.

Substituted alkyl groups are alkyl groups as described above which are substituted in one or more positions by, e.g., deuterium, halogen, hydroxyl, amino, carboxy, alkylamino, dialkylamino, aryl, heteroaryl, alkoxy, nitro and cyano, and combinations thereof.

The term “halogenated alkyl” means a saturated hydrocarbon radical which may be straight-chain or branched-chain and may comprise about 1 to about 20 carbon atoms, for instance 1 to 12 carbon atoms, such as 1 to 8 carbon atoms, e.g., 1 to 4 carbon atoms, that is substituted by one ore more halogens, such as, but not limited to, —CF₃, CF₂CF₃, CHF₂, CH₂F, and the like. The use of the term “halogenated alkyl” should not be construed to mean that a “substituted alkyl” group may not be substituted by one or more halogens.

The term “alkenyl” means a substituted or unsubstituted hydrocarbon radical which may be straight-chain or branched-chain, which contains one or more carbon-carbon double bonds, and which may comprise about 1 to about 20 carbon atoms, such as 1 to 12 carbon atoms, for instance 1 to 6 carbon atoms. Suitable alkenyl groups include ethenyl, propenyl, butenyl, etc.

Substituted alkenyl groups are alkenyl groups as described above which are substituted in one or more positions by, e.g., halogen, hydroxyl, amino, carboxy, alkylamino, dialkylamino, aryl, heteroaryl, alkoxy, nitro and cyano, and combinations thereof.

The term “alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

The term “alkynyl” means a substituted or unsubstituted aliphatic hydrocarbon radical which may be straight-chain or branched-chain and which contains one or more carbon-carbon triple bonds. Preferably the alkynyl group contains 2 to 15 carbon atoms, such as 2 to 12 carbon atoms, e.g., 2 to 8 carbon atoms. Suitable alkynyl groups include ethynyl, propynyl, butynyl, etc.

Substituted alkynyl groups are alkynyl groups as described above which are substituted in one or more positions by, e.g., halogen, hydroxyl, amino, carboxy, alkylamino, dialkylamino, aryl, heteroaryl, alkoxy, nitro and cyano, and combinations thereof.

The term “amino” means substituted or unsubstituted —NH₂. Substituted amino means a hydrogen substituted in one or more positions by, for example, but not limited to cyano, heterocycle, or heteroaryl group.

The term “alkylamino” means —NH(alkyl), wherein alkyl is optionally substituted as described above. Substituted alkyl includes, for example, but not limited to deuterium.

The term “dialkylamino” means —N(alkyl)₂, wherein alkyl is substituted as described above.

The term “aryl” means a substituted or unsubstituted aromatic monocyclic or bicyclic ring system comprising about 5 to about 14 carbon atoms, e.g., about 6 to about 10 carbon atoms. Suitable aryl groups include, but are not limited to, phenyl, naphthyl, anthracenyl.

Substituted aryl groups include the above-described aryl groups which are substituted one or more times by, for example, but not limited to, halogen, hydroxyl, amino, carboxy, alkylamino, dialkylamino, aryl, heteroaryl, alkoxy, nitro and cyano, and combinations thereof.

The term “arylamino” means —NH(aryl), wherein aryl is as described above.

The term “diarylamino” means —N(aryl)₂, wherein aryl is as described above.

The term “amido” means —CONH₂.

The term “aminoalkyl” means a -(alkylene)-amino, -(alkylene)-alkylamino or -(alkylene)-dialkylamino group, wherein the various groups are as described above.

The term “arylalkyl” refers to an -(alkylene)-aryl group in which the aryl and alkylene portions are in accordance with the previous descriptions. Suitable examples include, but are not limited to, benzyl, 1-phenethyl, 2-phenethyl, phenpropyl, phenbutyl, phenpentyl, and napthylmethyl.

The term “substituted arylalkyl” refers to -(alkylene)-aryl group in which the alkylene hydrogens are substituted with, for example, but not limited to, hydroxyl, fluoro or difluoro groups.

The term “carboxyl” means —C(O)OH.

The term “cycloalkyl” means a monocyclic, bicyclic or tricyclic nonaromatic saturated hydrocarbon radical having 3 to 10 carbon atoms, such as 3 to 8 carbon atoms, for example, 3 to 6 carbon atoms. Suitable cycloalkyl groups include, but are not limited to, cyclopropyl, cyclobutyl, cyclopentyl, cyclohexyl, cycloheptyl, cyclooctyl, norbornyl, 1-decalin, adamant-1-yl, and adamant-2-yl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, bicyclo[4.2.0]octyl, and spiro[3.5]nonyl. Preferred cycloalkyl groups include cyclopropyl, cyclobutyl, cyclopentyl and cyclohexyl. The cycloalkyl group can be substituted, for example, by one or more halogens and/or alkyl groups.

The term “cycloalkylalkyl” means a -(alkylene)-cycloalkyl in which the cycloalkyl group is as previously described; e.g., cyclopropylmethyl, cyclobutylmethyl, cyclopentylethyl, or cyclohexylmethyl, and the like.

The term “heteroaryl” means a substituted or unsubstituted aromatic monocyclic or multicyclic ring system comprising 5 to 14 ring atoms, preferably about 5 to about 10 ring atoms and most preferably 5 or 6 ring atoms, wherein at least one of the ring atoms is an N, O or S atom. Suitable heteroaryl groups include, but are not limited to furyl, thienyl, pyrrolyl, pyrazolyl, imidazolyl, pyridyl, pyrimidinyl, benzimidazolyl, indazolyl, indolyl, quinolinyl, isoquinolinyl, naphthyridinyl and the like.

Substituted heteroaryl groups include the above-described heteroaryl groups which are substituted one or more times by, for example, but not limited to, halogen, hydroxyl, amino, carboxy, alkyl, alkylamino, dialkylamino, aryl, heteroaryl, alkoxy, nitro and and combinations thereof.

The term “heteroarylalkyl” refers to a -(alkylene)-heteroaryl group wherein the heteroaryl and alkylene portions are in accordance with the previous discussions. Suitable examples include, but are not limited to, pyridylmethyl, thiazolylmethyl, thienylmethyl, pyrimidinylmethyl, pyrazinylmethyl, and isoquinolinylmethyl, and the like.

The term “heterocycle” means a substituted or unsubstituted non-aromatic mono- or multicyclic ring system comprising 3 to 10 atoms, preferably 5 or 6, wherein at least one of the ring atoms is an N, O or S atom. Suitable heterocyle groups include, but are not limited to tetrahydrofuranyl, tetrahydrothienyl, tetrahydropyranyl, dihydropyranyl, pyrrolidinyl, piperidinyl, piperazinyl, thiomorpholinyl, morpholinyl, isoxazolinyl, and the like.

Substituted heterocycle groups include the above-described heterocycle groups which are substituted one or more times by, for example, halogen, amino, alkyl, hydroxy, carboxy, and combinations thereof. Heterocycle groups may also be substituted by, e.g., aryl or heteroaryl.

The term “heterocyclealkyl” refers to a -(alkylene)-heterocycle group wherein the heterocycle and alkylene portions are in accordance with the previous discussions.

The term “aroyl” means an aryl-C(O)—, in which the aryl group is as previously described. Suitable aroyl groups include, but are not limited to, benzoyl and 1-naphthoyl.

The term “acyl” means an HC(O)—, alkyl-C(O)—, cycloalkyl-C(O)—, aryl-C(O)—, or heteroalkyl-C(O)—, in which the various groups are as previously described, e.g., acetyl, propionyl, benzoyl, pyridinylcarbonyl, and the like.

The term “alkoxy” means alkyl-O— groups in which the alkyl portion can be optionally substituted in accordance with the previous discussion. Suitable alkoxy groups include, but are not limited to, methoxy, ethoxy, n-propoxy, iso-propoxy, n-butoxy, t-butoxy, pentoxy, hexoxy, heptoxy, octoxy, and the like. For example, the alkoxy can be methoxy or ethoxy. For example, suitable substituted alkoxy can be isopropoxy or difluoromethoxy.

The term “aryloxy” means an aryl-O— group, in which the aryl group is as previously described.

The term “heteroaryloxy” means an heteroaryl-O— group, in which the heteroaryl group is as previously described.

The term “cycloalkylalkyloxy” means a —O-(alkylene)-cycloalkyl group, in which the cycloalkyl and alkylene groups are as previously described.

The term “alkylthio” means an alkyl-S— group, in which the alkyl group is as previously described.

The term “arylthio” means an aryl-S— group, in which the aryl group is as previously described.

The term “alkylsulfinyl” means a —SOR radical where R is alkyl as defined above, e.g., methylsulfinyl, ethylsulfinyl, and the like.

The term “alkylsulfonyl” means a —SO₂R radical where R is alkyl as defined above, e.g., methylsulfonyl, ethylsulfonyl, and the like.

The term “arylsulfinyl” means a —SOR radical where R is aryl as defined above, e.g., phenylsulfinyl, and the like.

The term “arylsulfonyl” means a —SO₂R radical where R is aryl as defined above, e.g., phenylsulfonyl, and the like.

The term “heteroarylsulfinyl” means a —SOR radical where R is heteroaryl as defined above.

The term “heteroarylsulfonyl” means a —SO₂R radical where R is heteroaryl as defined above.

The term “alkoxycarbonyl” means an alkyl-O—C(O)— group, in which the alkyl group is as previously described.

The term “aryloxycarbonyl” means an aryl-O—C(O)— group, in which the aryl group is as previously described.

The term “heteroaryloxycarbonyl” means an heteroaryl-O—C(O)— group, in which the heteroaryl group is as previously described.

The term “cycloalkyloxy” means a —O-cycloalkyl group in which the cycloalkyl group is as previously described, e.g., cyclopropyloxy, cyclobutyloxy, cyclopentyloxy, cyclohexyloxy, and the like.

The term “arylalkyloxy” means —O-(alkylene)-aryl group, in which the aryl and alkylene groups are as previously described.

The term “heteroarylalkyloxy” means —O-(alkylene)-heteroaryl group, in which the heteroaryl and alkylene groups are as previously described.

One of ordinary skill in the art will recognize that compounds of formulas I-IV can exist in different tautomeric and geometrical isomeric forms. All of these compounds, including cis isomers, trans isomers, diastereomic mixtures, racemates, nonracemic mixtures of enantiomers, substantially pure, and pure enantiomers, are within the scope of the present invention. Substantially pure enantiomers contain no more than 5% w/w of the corresponding opposite enantiomer, such as no more than 2%, for example, no more than 1%.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. Optically active compounds of formulas I-IV can likewise be obtained by utilizing optically active starting materials in chiral synthesis processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] (2000), 110 pp. CAN 133:68895 AN 2000:473538 CAPLUS; Kabalka, George W.; Varma, Rajender S. The synthesis of radiolabeled compounds via organometallic intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem. (1981), 64(1-2), 9-32. CODEN: JRACBN ISSN:0022-4081, CAN 95:76229 AN 1981:476229 CAPLUS.

Where applicable, the present invention also relates to useful forms of the compounds as disclosed herein, such as base free forms, and pharmaceutically acceptable salts or prodrugs of all the compounds of the present invention for which salts or prodrugs can be prepared. Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, aDIPEAtes, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride, a hydrobromide, a hydroformate, or a maleate. For further example, the pharmaceutically acceptable salt is a hydrochloride.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of formulas I-IV can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of formulas I-IV can exist in different solvate forms. Solvates of the compounds of the invention may also form when solvent molecules are incorporated into the crystalline lattice structure of the compound molecule during the crystallization process.

The present invention also includes prodrugs of compounds of formulas I-IV. The term prodrug is intended to represent covalently bonded carriers, which are capable of releasing the active ingredient of formulas I-IV when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of formulas I-IV include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of formulas I-IV), amides (e.g., trifluoroacetylamino, acetylamino, and the like), and the like. Prodrugs of compounds of formulas I-IV are also within the scope of this invention.

The present invention also provides processes for preparing the compounds of formulas I-IV. Suitable general reaction schemes are shown below.

Compounds of formula I may be prepared by amide coupling between appropriately substituted carboxylic acids and amines as depicted in general synthetic schemes 1-5.

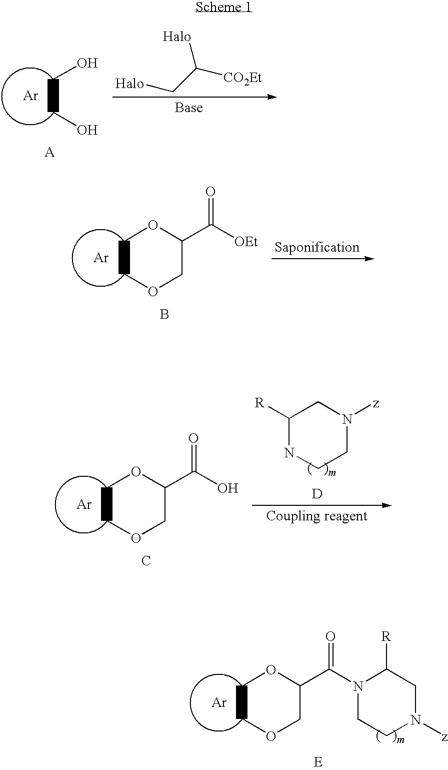

Precursor A in Scheme 1 (where Ar is an aromatic ring, e.g., phenyl) is treated with a dihaloalkyl ester in the presence of a base such as potassium tert-butoxide to obtain compound B as a mixture of region- and stereo-isomeric products. Saponification of compound B followed by amide coupling with an appropriately substituted piperazine or homopiperizine unit D using suitable coupling agents (e.g., EDC/HOBt) affords the desired compound E.

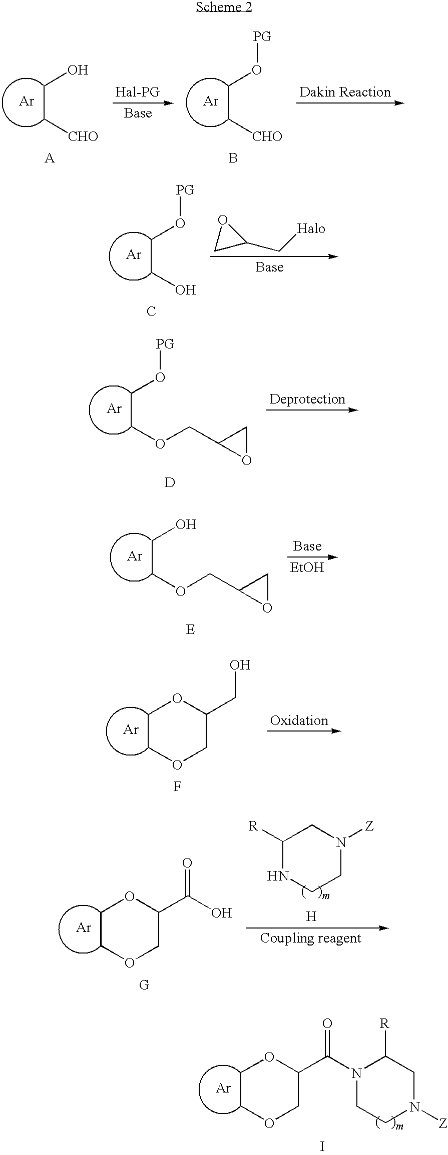

Protection of the free hydroxyl group of precursor A in Scheme 2 with a suitable protecting agent (PG, such as benzyl chloride) affords compound B. Conversion of compound B to C is accomplished via a Dakin reaction using hydrogen peroxide and sulfuric acid. Compound C is then reacted with epi-chlorohydrin in the presence of a base such as cesium carbonate to obtain compound D. Reductive deprotection of Compound D with hydrogen/Pd/C affords Compound E. On treatment with strong alkali such as sodium hydroxide, Compound E affords Compound F as a pure regio-siomer, which is oxidized with e.g., CrO₃/H₂SO₄, to afford Compound G as a pure regio-isomer. Amide coupling between Compound G and an appropriately substituted piperazine or homopiperazine derivative (H) using suitable coupling agents (e.g., EDC/HOBt) affords the desired Compound I.

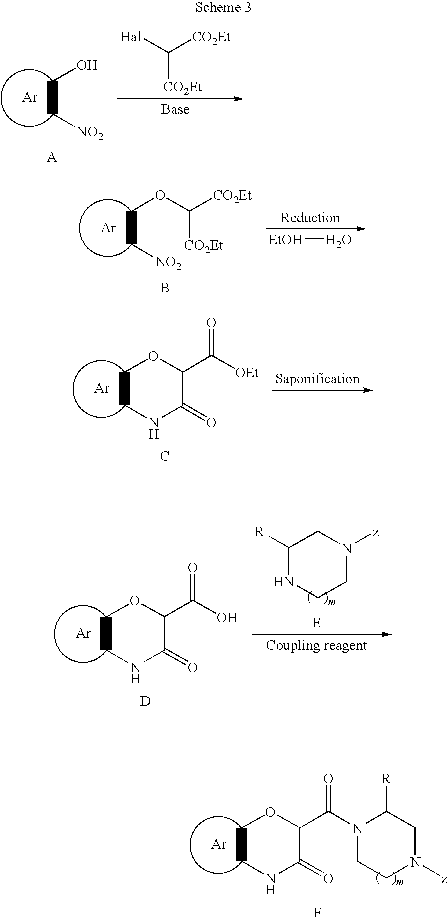

Compound B in Scheme 3 is prepared by reacting precursor A with an α-haloalkyl diester in the presence of a suitable base such as potassium tert-butoxide. Subjecting Compound B to reductive conditions with iron affords Compound C, which upon saponofication yields Compound D. Amide coupling between Compound D and an appropriately substituted piperazine or homopiperazine derivative (E) using suitable coupling agents (e.g., EDC/HOBt) affords the desired Compound F.

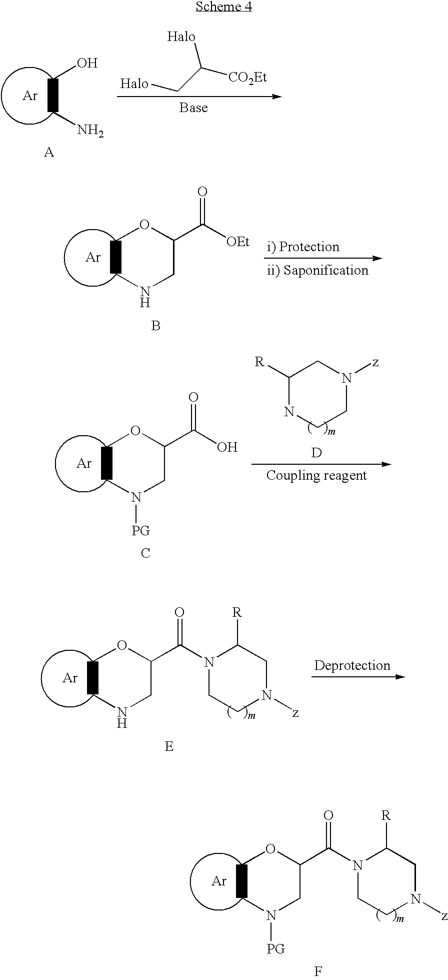

Compound B in Scheme 4 is prepared by reacting precursor A with an appropriate dihaloalkyl ester in the presence of a suitable base such as potassium tert-butoxide. Protection of the basic nitrogen in Compound B using a protecting group (PG, such as BOC) followed by saponification affords Compound C. Alternatively Compound B in Scheme 4 may be prepared by reducing Compound C of scheme 3 with e.g., borane.

Amide coupling between Compound C and an appropriately substituted piperazine or homopiperazine derivative (D) using suitable coupling agents (e.g., EDC/HOBt) followed by subsequent removal of protecting group (PG) affords the desired Compound F.

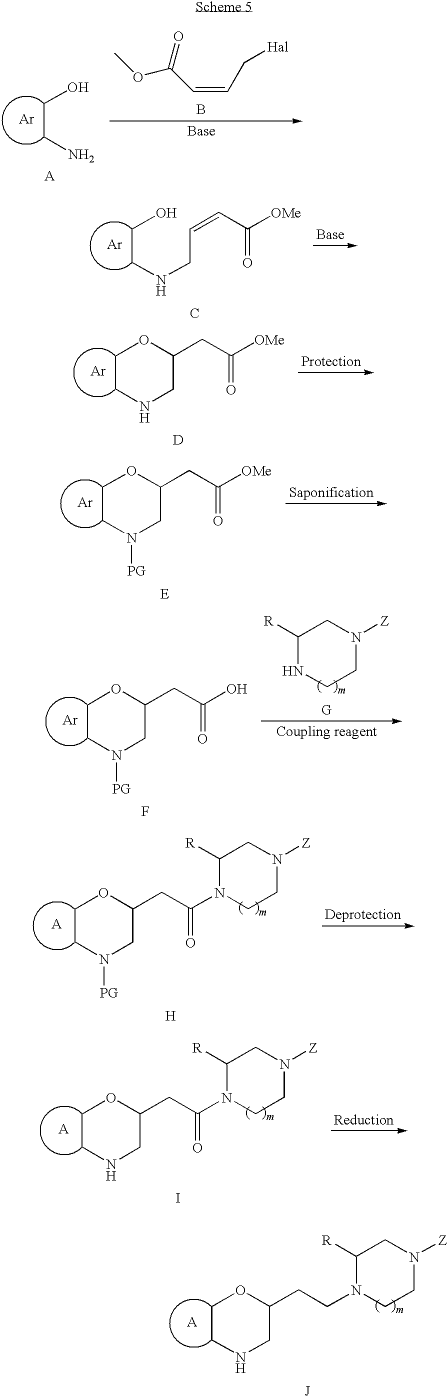

Compound C in scheme 5 is prepared by reacting Compound A with Compound B in presence of a suitable base such as potassium tert-butoxide. Further treatment of compound C with base affords Compound D. Protection of the free nitrogen of Compound D, for example, as a tert-butyl carbamate derivative (Compound E), followed by saponification affords Compound F. Amide coupling between Compound F and an appropriately substituted piperazine or homopiperazine derivative (G) using suitable coupling agents (e.g., EDC/HOBt) followed by subsequent removal of protecting group (PG) affords the desired Compound I. Reduction of Compound I using borane affords Compound J.

Compounds of formula II may be prepared by amide coupling between appropriately substituted carboxylic acids and amines as depicted in general synthetic schemes 6 and 7.

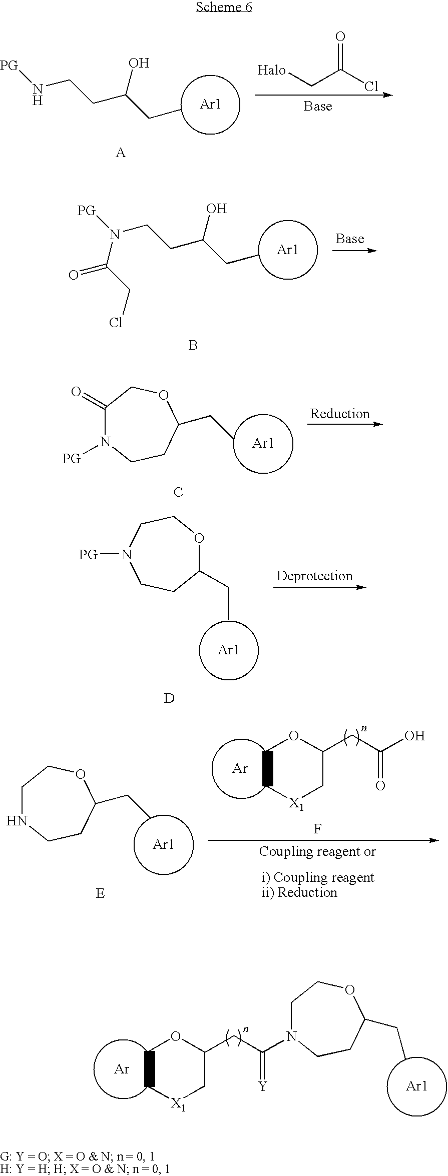

Precursor A in scheme 6 may be prepared following the synthetic method described in J. Am. Chem. Soc., 125, 10502-10503, 2003, using appropriate starting materials (PG=protecting group). Reaction of precursor A with a haloalkyl acid chloride affords Compound B, which is cyclised in presence of an anhydrous base such as sodium hydride to afford Compound C. Reduction of Compound C with borane followed by deprotection affords Compound E. Amide coupling using an appropriate combination of Compounds E and F using suitable coupling agents (e.g., EDC/HOBt) affords Compound G. Reduction of Compound G using borohydride reducxing agents affords Compound H. Carboxylic acids F may be prepared following the synthetic route described for Compound I in scheme 5.

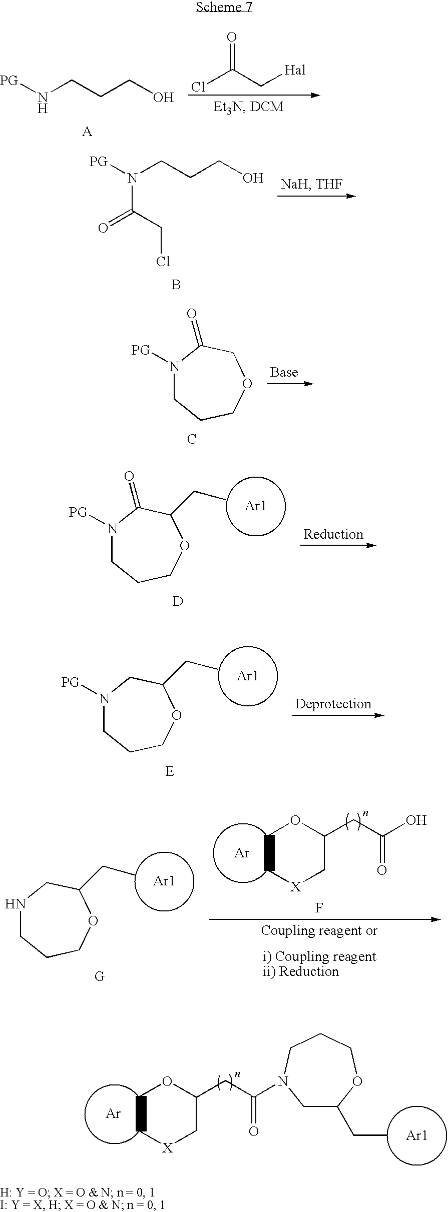

Precursor B in scheme 7 is prepared by reacting Compound A with an appropriate haloalkyl acid chloride in presence of a base such as triethylamine. Cyclization of Compound B with sodium hydride followed by C-alkylation of corresponding lithium enolate affords Compound D, which on reduction with a metal hydride affords Compound E. N-Deprotection of

Compound E followed by amide coupling with appropriate Compound F using suitable coupling agents (e.g., EDC/HOBt) affords Compound H. Reduction of Compound H with, e.g., borane affords Compound I. Appropriate carboxylic acids (Compound F) may be prepared following the synthetic route described for Compound I in scheme 5.

Compounds of formula III may be prepared by amide coupling between appropriate carboxylic acids and amines as depicted in general synthetic scheme 8.

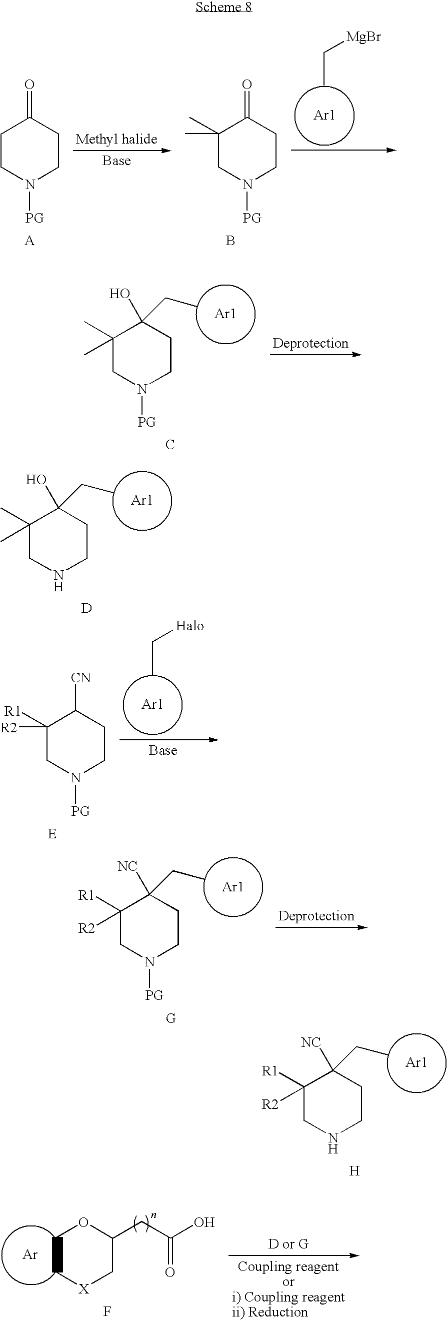

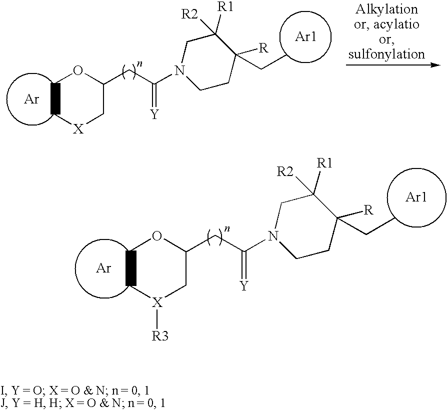

Compound B in Scheme 8 is prepared by alkylation of the enolate of Compound A. Reaction of Compound B with ab appropriate Grignard reagent affords Compound C, which after removal of protecting group (PG) affords Compound D. Base assisted alkylation of Compound E affords Compound G, which undergoes N-deprotection with anhydrous acid to afford Compound H. Appropriate precursor Compounds F are prepared following the synthetic route described for Compound I in scheme 5.Amide coupling of Compound F with either Compound D or Compound G, using suitable coupling reagents (e.g., EDC/HOBt) affords Compound I. Compound J is prepared by reduction of the amide bond of Compound I with e.g., borane. Alkylation, acylation or sulfonylation of the Compound I or Compound J (X═N) affords Compound K.

Compounds of formula IV may be prepared by amide coupling between appropriate carboxylic acids and amines as depicted in general synthetic scheme 9.

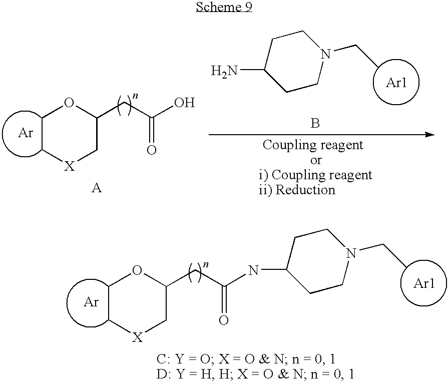

Appropriate precursors A in Scheme 9 are prepared following the synthetic route described for Compound I in scheme 5. Amide coupling, using suitable coupling reagents (e.g., EDC/HOBt) with an appropriate aminopiperidine unit (Compound B) affords Compound C. Reduction of the amide bond of Compound C using e.g., borane affords Compound D.

The compounds of the invention can be administered alone or as an active ingredient of a formulation. Thus, the present invention also includes pharmaceutical compositions of compounds of formulas I, II, III or IV, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Administration of the compounds of the present invention may be accomplished according to patient needs, for example, orally, nasally, parenterally (subcutaneously, intraveneously, intramuscularly, intrasternally and by infusion) by inhalation, rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, the compounds of formulas I, II, III or IV can be administered by inhalation in the form of a powder (e.g., micronized) or in the form of atomized solutions or suspensions. The aerosol formulation can be placed into a pressurized acceptable propellant.

### Methods of Treatment

The compounds of formulas I, II, III or IV may be useful as ligands for CC chemokine receptors, for example, CCR1. Therefore, compounds of formulas I, II, III or IV may be useful in the treatment of conditions mediated by CC chemokine receptors, for example, CCR1. In certain embodiments, the compounds of the present invention may be useful in the treatment of conditions that respond to a CCR1 receptor agonist, inverse agonist or antagonist. For example, conditions that respond to a CCR1 antagonist.

In yet another aspect, the present invention provides methods of treating CC chemokine receptor (e.g., CCR1) mediated conditions or diseases by administering to a patient having such a disease or condition, a therapeutically effective amount of a compound of formulas I, II, III or IV above.

CCR1 provides a target for interfering with or promoting specific aspects of immune cell functions, or more generally, with functions associated with CCR1 expression on a wide range of cell types in a mammal, such as a human. Compounds that inhibit CCR1, are particularly useful for modulating monocyte, macrophage, lymphocyte, granulocyte, NK cell, mast cells, dendritic cell, neutrophils, and certain immune derived cell (for example, osteoclasts) function for therapeutic purposes. Accordingly, the present invention is directed to compounds which are useful in the prevention and/or treatment of a wide variety of inflammatory and immunoregulatory disorders and diseases (see Saeki, et al., Current Pharmaceutical Design, 9, 1201-1208, 2003).

There are also provided, in accordance with embodiments of the invention, methods of treating or preventing inflammatory or autoimmune diseases comprising administering a compound of formulas I, II, III or IV. In some embodiments the inflammatory disease or autoimmune disease is rheumatoid arthritis or multiple sclerosis.

Compounds of the genera are CCR1 antagonists. As such, they have utility in treating and preventing autoimmune disease and inflammatory diseases. In particular, CCR1 antagonists are therapeutic targets for the treatment and prevention of a variety of diseases, including, but not limited to, autoimmune diseases (such as rheumatoid arthritis, Takayasu arthritis, psoriatic arthritis, ankylosing spondylitis, type 1 diabetes (recent onset), lupus, inflammatory bowel disease, Crohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulo interstitial fibrosis, subepithelial fibrosis, scleroderma (progressive systemic sclerosis), hepatic fibrosis (including that caused by alcoholic or viral hepatitis), primary and secondary biliary cirrhosis); diabetic nephropathy; allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xenotransplantation); HIV Infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease and other neurodegenerative diseases; osteolytic lesion and sequelae associated with certain cancers such as multiple myeloma; diagnosis and treatment of endometriosis; analgesia. Compounds of formulas I, II, III or IV may also inhibit the production of metallo proteinases and cytokines at inflammatory sites (including but not limited to MMP9, TNF, IL-I, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). Compounds of formulas I, II, III or IV may also prevent tissue damage caused by inflammation induced by infectious agents (such as viral induced encephalomyelitis or demyelination, viral inflammation of the lung or liver (e.g. caused by influenza or hepatitis or respiratory syncytial virus), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria), arterial remodeling characterized by neointima formation and medial thickening for mediating inflammatory cell recruitment and endothelial dysfunction.

There are also provided, in accordance with additional embodiments of the invention, methods of treating or preventing a disease disease or condition selected from, for example, hepatocellular carcinoma, respiratory synctial virus (RSV), kidney disease, allergic asthma, Alport disease (which includes glumerulosclerosis and progressive renal fibrosis), prion diseases, sepsis, T-cell mediated liver diseases, severe respiratory viruses, chronic renal injury, and transplant and cardio allograft vascalopathy (chronic rejection) comprising administering a compound of formulas I, II, III or IV. In some embodiments the Alport disease is renal fibrosis.

There are also provided, in accordance with embodiments of the invention, methods of treating or preventing endometriosis comprising administering a compound of formulas I, II, III or IV.

In certain embodiments, the compounds of the invention are useful in the treatment of elevated levels of lipids, cardiovascular diseases, diabetes, obesity, and metabolic syndrome.

In the treatment or prevention of conditions which require chemokine receptor modulation an appropriate dosage level will generally be about 0.001 to 100 mg per kg patient body weight per day which can be administered in single or multiple doses. For example, the dosage level will be about 0.01 to about 25 mg/kg per day; such as about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions may be provided in the form of tablets containing about 1 to about 1000 milligrams of the active ingredient, such as about 1, about 5, about 10, about 15, about 20, about 25, about 50, about 75, about 100, about 150, about 200, about 250, about 300, about 400, about 500, about 600, about 750, about 800, about 900, and about 1000 milligrams of the active ingredient. The compounds may be administered on a regimen of 1 to 4 times per day, for example, once or twice per day.

It will be understood, however, that the specific dose level and frequency of dosage for any particular patient may be varied and will depend upon a variety of factors including the activity of the specific compound employed, the metabolic stability and length of action of that compound, the age, body weight, hereditary characteristics, general health, sex and diet of the subject, as well as the mode and time of administration, rate of excretion, drug combination, and the severity of the particular condition for the subject undergoing therapy.

The compounds and compositions of the present invention can be combined with other compounds and compositions having related utilities to prevent and treat the conditions described herein, such as, for example, inflammatory or autoimmune disorders, conditions and diseases, including inflammatory bowel disease, rheumatoid arthritis, osteoarthritis, psoriatic arthritis, polyarticular arthritis, multiple sclerosis, allergic diseases, psoriasis, atopic dermatitis and asthma.

For example, in the treatment or prevention of inflammation or autimmunity or for example arthritis associated bone loss, the present compounds and compositions may be used in conjunction with, for example, an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non steroidal anti-inflammatory agent, or a cytokine-suppressing anti-inflammatory agent, for example with a compound such as acetaminophen, aspirin, codeine, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds and compositions may be administered with an analgesic listed above; a potentiator such as caffeine, an H2 antagonist (e.g., ranitidine), simethicone, aluminum or magnesium hydroxide; a decongestant such as phenylephrine, phenylpropanolamine, pseudoephedrine, oxymetazoline, ephinephrine, naphazoline, xylometazoline, propylhexedrine, or levo desoxy ephedrine; an antitussive such as codeine, hydrocodone, caramiphen, carbetapentane, or dextromethorphan; a diuretic; and a sedating or non sedating antihistamine.

Such other drug(s) may be administered, by a route and in an amount commonly used therefor, contemporaneously or sequentially with a compound of formulas I, II, III or IV. When a compound of formulas I, II, III or IV is used contemporaneously with one or more other drugs, a pharmaceutical unit dosage form containing such other drugs in addition to the compound of formulas I, II, III or IV may be employed. Accordingly, the pharmaceutical compositions of the present invention include those that also contain one or more other active ingredients, in addition to a compound of formulas I, II, III or IV.

The term “treating” means to relieve, alleviate, delay, reduce, reverse, improve or prevent at least one symptom of a condition in a subject. The term “treating” may also mean to arrest, delay the onset (i.e., the period prior to clinical manifestation of a disease) and/or reduce the risk of developing or worsening a condition.

An “effective amount” means the amount of a compound of formulas I, II, III or IV that, when administered to a patient (e.g., a mammal) for treating a disease, is sufficient to effect such treatment for the disease to achieve the objectives of the invention. The “effective amount” will vary depending on the compound, the disease and its severity and the age, weight, etc., of the patient to be treated.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, such as up to 10%, for example, up to 5%.

### Examples

The present invention will now be further described by way of the following non-limiting examples. In applying the disclosure of these examples, it should be kept clearly in mind that other and different embodiments of the methods and synthetic schemes disclosed according to the present invention will no doubt suggest themselves to those of skill in the relevant art.

In the foregoing and in the following examples, all temperatures are set forth uncorrected in degrees Celsius; and, unless otherwise indicated, all parts and percentages are by weight.

The following abbreviations may be used herein: Ac (CH₃CO), Bn (benzyl), DCM (dichloromethane), DMF (dimethylformamide), DIPEA (N,N-diisopropyl ethyl amine), EDCI (1-ethyl-3-[3-dimethylaminopropyl]carbodiimide hydrochloride), Et (ethyl), HOBT (1-hydroxybenzotriazole), Me (methyl), TFA (trifluoroacetic acid), THF (tetrahydrofuran), EtOH (ethanol), EtOAc (ethyl acetate), MeOH (methanol), K₂CO₃ (potassium carbonate), Pd/C (palladium on carbon), Boc (tert-butoxycarbonyl), Na₂SO₄ (sodium sulphate), CHCl₃ (chloroform), NaOH (sodium hydroxide), DMAP (dimethyl amino pyridine), NMR (nuclear magnetic resonance), DMSO-d₆ (deuterated dimethyl sulfoxide), CDCl₃ (deuterated chloroform), LC-MS (liquid chromatography-mass spectrometry), LDA (lithium diisopropylamine), HPLC (high pressure liquid chromatography or high performance liquid chromatography), Me₂NH (dimethyalamine) RT (room temperature), Nall (sodium hydride), tBuOK (potassiume tert-butoxide), NH₄Cl (ammonium chloride), LiOH (lithium hydroxide), H₂O₂ (hydrogen peroxide), NaHCO₃ (sodium hydrogen carbonate), DMS (dimethyl sulfoxide), GCMS (gas chromatography-mass spectrometry), Si-gel (silica gel), Na₂S₂O₄ (sodium hydrosulfite), TBTU (O-(benzotriazol-1-yl)-N,N,N′,N′-tetramethyluronium tetrafluoroborate), Jones Reagent (chromium trioxide in aqueous sulfuric acid).

### Example 1

### (6-Chloro-2,3-dihydro-benzo[1,4]dioxin-2-yl)-[(R)-4-(4-fluoro-benzyl)-2-methyl-piperazin-1-yl]-methanone

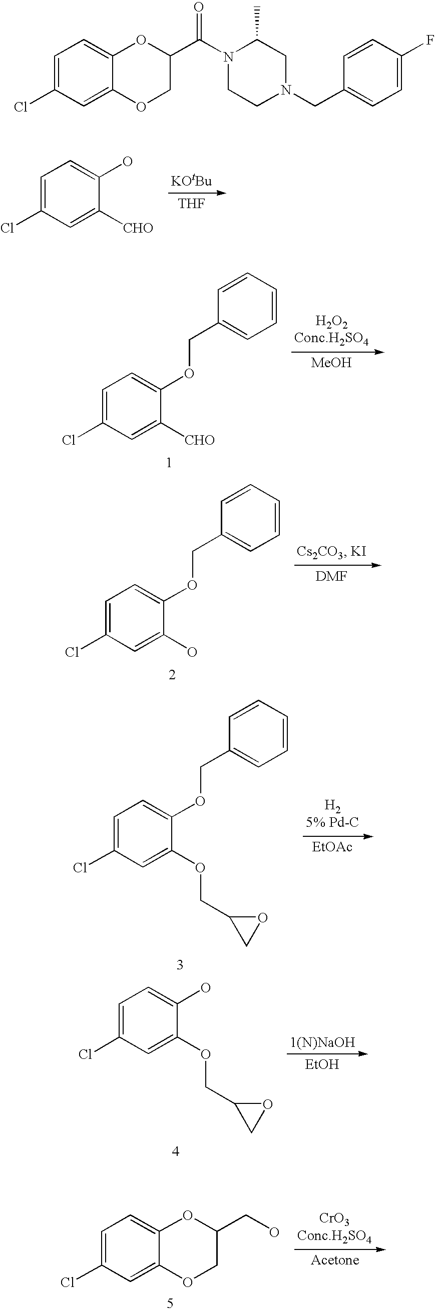

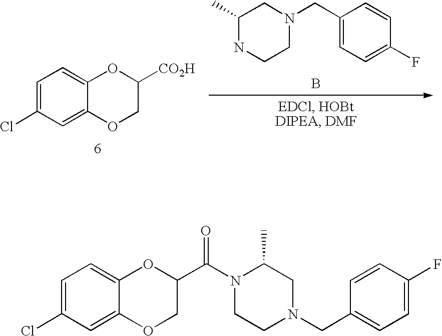

Preparation of (R)-1-(4-fluoro-benzyl)-3-methyl-piperazine

A mixture of (R)-2-Methyl-piperazine (2 g, 0.02 mol), 4-fluorobenzyl bromide (2.38 ml, 0.02 mol), and sodium bicarbonate (5.04 g, 0.06 mol) in ethanol was refluxed for 24 hours. The solution was filtered and the filtrate was evaporated under reduced pressure. The residue was diluted with water and extracted with ethyl acetate. The organic layer was washed with brine solution and dried over sodium sulfate. The crude was purified over neutral alumina (5% ethyl acetate-hexane) to afford 1.2 g (29%) of (R)-1-(4-fluoro-benzyl)-3-methyl-piperazine. LC/MS [M+H⁺: 209.3. ¹H-NMR (400 MHz, CDCl₃) δ (ppm): 7.27-6.96 (m, 4 Ar—H,), 3.43 (s, 2H), 2.96-2.82 (m, 3H)), 2.73 (d, J=11.5 Hz, 2H), 2.00 (m, 1H), 1.65 (m, 1H), 1.00 (d, J=6.6 Hz, 3H).

Step-1: 2-Benzyloxy-5-chloro-benzaldehyde

To a solution of 5-Chloro-2-hydroxy-benzaldehyde (1 g, 6.4 mmol) in THF was added potassium tert- butoxide (788 mg, 7 mmol) portion wise at 0° C. and stirred at room temperature for 2 h. Benzyl bromide (0.76 ml, 6.4 mmol) was added to the reaction solution at 0° C. and refluxed for 4 h. The reaction mixture was concentrated, diluted with water, and the product was extracted with ethyl acetate. Concentration of ethyl acetate layer in vacuo afforded 1.6 g (99%) of 2-Benzyloxy-5-chloro-benzaldehyde. GCMS [m/z]:246.0. ¹H-NMR (400 MHz, DMSO-d₆) δ (ppm): 10.34 (s, 1H), 7.72-7.33 (m, 8 Ar H), 5.31 (s, 2H),

Step-2: 2-benzyloxy-5-chlorophenol

2-Benzyloxy-5-chloro-benzaldehyde (200 mg, 0.81 mmol) was dissolved in methanol and treated with hydrogen peroxide (0.36 ml, 30%) and conc. sulfuric acid (0.17 ml). The mixture was stirred overnight at room temperature and evaporated. The residue was dissolved in saturated aq. NaCl solution and extracted with ether. The organic phase was dried over sodium sulfate and concentrated under reduced pressure. Purification of the residue by flash chromatography (silica-gel, 2% EtOAc-hexane) afforded 85 mg (45%) of 2-benzyloxy-5-chlorophenol. GCMS [m/z]:234.

¹H-NMR (400 MHz, DMSO-d₆) δ (ppm): 9.56 (s, 1H), 7.46-6.72 (m, 8 Ar H), 5.09 (s, 2H).

Step-3: 2-(2-Benzyloxy-5-chloro-phenoxymethyl)-oxirane

2-benzyloxy-5-chlorophenol 2 (200 mg, 8.5 mmol) was dissolved in DMF and treated with epi-chlorohydrine (118 mg, 1.3 mmol) using a catalytic amount of KI and cesium carbonate (415 mg, 1.3 mmol). The mixture was stirred at room temperature for 24 h. The reaction mixture was diluted with water, extracted with ether, and the organic layer was dried over Na₂SO₄ and finally concentrated under reduce pressure to afford 250 mg (99%) of 2-(2-benzyloxy-5-chloro-phenoxymethyl)-oxirane. GCMS [m/z]: 290. ¹H-NMR (400 MHz, CDCl₃) δ (ppm): 7.42-6.8 (m, 8 Ar H), 5.08 (s, 2H), 4.26 (dd, J=8.24, 3.04 Hz 1H), 3.98 (m, 1H), 3.35 (m, 1H), 2.88 (m, 1H), 2.75 (m, 1H).

Step-4: 4-Chloro-2-oxiranylmethoxy-phenol

2-(2-Benzyloxy-5-chloro-phenoxymethyl)-oxirane (100 mg, 3.4 mmol) was hydrogenated with 5% Pd-C (40 mg) in ethyl acetate for 1.5 h. The catalyst was filtered through a celite bed. Concentration of the ethyl acetate solution afforded 50 mg (99%) of 4-chloro-2-oxiranylmethoxy-phenol. GCMS [m/z]: 200. ¹H-NMR (400 MHz, CDCl₃) δ (ppm): 6.93-6.81 (m, 3 Ar H), 4.30 (dd, J=3.8, 2.4 Hz, 1H), 4.0 (m, 1H), 3.36 (m, 1H), 2.95 (t, J=4.4 Hz, 1H), 2.82 (m, 1H).

Step-5: (6-Chloro-2,3-dihydro-benzo[1,4]dioxin-2-yl)-methanol

4-Chloro-2-oxiranylmethoxy-phenol (500 mg, 2.5 mmol) was dissolved in ethanol and treated with 2.6 ml of aq. 1(N) NaOH solution. The mixture was stirred for 30 min. and diluted with ether. The organic phase was washed with 1(N) HCl solution and water, dried over sodium sulfate and concentrated to afford 420 mg (70%) of (6-chloro-2,3-dihydro-benzo[1,4]dioxin-2-yl)-methanol. ¹H-NMR (400 MHz, CDCl₃), δ (ppm): 6.93-6.82 (m, 3 Ar H), 4.27 (dd, J=9.1, 2.2 Hz, 1H), 4.21 (m, 1H), 4.08 (m, 1H), 3.89 (dd, J=7.89, 4.1 Hz, 1H), 3.8 (m, 1H).

Step-6: 6-Chloro-2,3-dihydro-benzo[1,4]dioxine-2-carboxylic acid

To a stirred solution of (6-Chloro-2,3-dihydro-benzo[1,4]dioxin-2-yl)-methanol (420 mg, 2.1 mmol) in acetone was added Jones reagent (1.35 ml) dropwise at 10-15° C. The reaction was stirred at room temperature for 3.5 h and then was diluted with chloroform. The organic layer was separated and evaporated. The residue was taken up in chloroform and was washed with saturated sodium bicarbonate solution. The aqueous phase was washed with chloroform, cooled, acidified with conc. HCl (pH 1) and was extracted with chloroform. Concentration of the chloroform layer afforded 105 mg (23%) of 6-Chloro-2,3-dihydro-benzo[1,4]dioxine-2-carboxylic acid. GCMS [m/z]: 214. ¹H-NMR (400 MHz, DMSO-d₆) δ (ppm): 13.46 (br s, 1H), 6.96 (m, 2H), 6.9 (dd, J=6.3, 2.4 Hz, 1H), 5.08 (t, J=2.9 Hz, 1H), 4.30 (dd, J=8.9, 3.2 Hz, 1H), 4.27 (dd, J=8.5, 2.8 Hz, 1H).

Step-7: (6-Chloro-2,3-dihydro-benzo[1,4]dioxin-2-yl)-[(R)-4-(4-fluoro-benzyl)-2-methyl-piperazin-1-yl]-methanone

To a solution of 6-chloro-2,3-dihydro-benzo[1,4]dioxine-2-carboxylic acid (500 mg, 2.3 mmol) in DMF was added (R)-1-(4-fluoro-benzyl)-3-methyl-piperazine (487 mg, 2.35 mmol), TBTU (827 mg, 2.6 mmol), and DIPEA (0.4 ml) at 5-10° C. and the resulting mixture was stirred at room temperature for 14-16 hours. The reaction mixture was diluted with water and the product was extracted with ethyl acetate. The ethyl acetate layer was concentrated in vacuo and subjected to column chromatography (45% ethyl acetate-hexane) to afford 450 mg (48%) of (6-chloro-2,3-dihydro-benzo[1,4]dioxin-2-yl)-[(R)-4-(4-fluoro-benzyl)-2-methyl-piperazin-1-yl]hmethanone. LC/MS [M+H]⁺: 405.2. ¹H-NMR (400 MHz, DMSO-d₆) δ (ppm): 7.35 (t, J=5.8 Hz, 2H), 7.16 (t, J=8.8 Hz, 2H), 6.88 (m, 3H), 5.21 (m, 1H), 4.47-3.79 (m, 7H), 2.91-1.87 (m, 5H), 1.18 (d, J=12.2 Hz, 3H). HPLC purity: 95.1%

### Example 2

### (2,3-Dihydro-benzo[1,4]dioxin-2-yl)-[(R)-4-(4-fluoro-benzyl)-2-methyl-piperazin-1-yl]-methanone

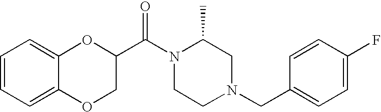

(6-Chloro-2,3-dihydro-benzo[1,4]dioxin-2-yl)-[(R)-4-(4-fluoro-benzyl)-2-methyl-piperazin-1-yl]-methanone (40.5 mg, 0.1 mmol) was hydrogenated with 10% Pd-C catalyst for 14 hours. The catalyst was removed by filtration through a celite bed. Concentration of the ethanol solution afforded 35 mg (85%) of (2,3-dihydro-benzo[1,4]dioxin-2-yl)-[(R)-4-(4-fluoro-benzyl)-2-methyl-piperazin-1-yl]-methanone. LC/MS [M+H]⁺: 371.4. ¹H-NMR (400 MHz, DMSO-d₆) δ (ppm): 7.36 (t, 2H), 7.16 (t, 2H), 6.88 (m, 4H), 5.15 (m, 1H) 4.48 (m, 1H), 4.38 (m, 1H)), 4.15 (m, 2H), 3.38 (m, 1H), 2.78 (m, 1H), 2.60 (m, 2H), 2.02 (m, 1H), 1.39 (d, 1H), 1.33 (m, 1H), 1.23 (m, 1H), 1.16 (t, 2H). HPLC purity: 97.1%

### Example 3

### (2,3-Dihydro-benzo[1,4]dioxin-2-yl)-[4-(4-fluoro-benzyl)-piperazin-1-yl]-methanone

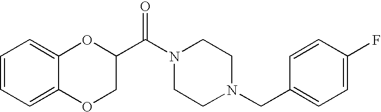

(2,3-Dihydro-benzo[1,4]dioxin-2-yl)-[4-(4-fluoro-benzyl)-piperazin-1-yl]-methanone was prepared from (6-chloro-2,3-dihydro-benzo[1,4]dioxin-2-yl)-[4-(4-fluoro-benzyl)-piperazin-1-yl]hmethanone by a process similar to that described in Example 2. LC/MS [M+H]⁺: 357.2. ¹H-NMR (400 MHz, DMSO-d₆) δ (ppm): 7.27 (m, 2H), 7.03 (t, 2H), 6.86 (m, 4H), 4.81 (dd, 1H), 4.48 (dd, 1H), 4.30 (m, 1H), 3.74 (m, 2H), 3.57 (m, 2H), 3.49 (s, 2H0, 2.44 (m, 4H). HPLC purity: 94.3%.

### Example 4

### (6-Chloro-2,3-dihydro-benzo[1,4]dioxin-2-yl)-[4-(4-fluoro-benzyl)-piperazin-1-yl]-methanone

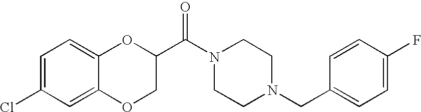

### Preparation of 1-(4-Fluoro-benzyl)-piperazine

To a solution of 1-boc-piperazine (1 g, 5.4 mmol) in THF was added 4-fluorobenzyl bromide (0.7 ml, 5.4 mmol) and triethylamine (2.2 ml, 16.1 mmol). The reaction mixture was stirred for 22 hour at room temperature. The reaction mixture was concentrated, diluted with water and the product extracted with ethyl acetate. The organic layer was washed with water and dried over sodium sulfate. The organic solution was concentrated to afford 1.6 g (100%) of 4-(4-fluoro-benzyl)-piperazine-1-carboxylic acid tert-butyl ester.

To a solution of 4-(4-fluoro-benzyl)-piperazine-1-carboxylic acid tert-butyl ester (1.6 g, 5.4 mmol) in DCM was added trifluoroacetic acid (4.16 ml, 54 mmol). The resulting mixture was stirred for about 5 h. The reaction mass was concentrated and the residue was washed with dry ether. The TFA salt was diluted with minimum volume of water and neutralized with aq. NaOH solution at 5-10° C. Extraction of the aqueous solution with ethyl acetate followed by concentration of the combined organic phases under reduced pressure afforded 1 g (95%) of 1-(4-Fluoro-benzyl)-piperazine. LC/MS [M+H]: 195.2. ¹H-NMR (400 MHz, DMSO-d₆) δ (ppm): 7.33-7.10 (m, 4 Ar H), 3.4 (s, 2H), 2.70 (t, J=4.8 Hz, 4H), 2.30 (t, J=1.6 Hz, 4 H).

(6-Chloro-2,3-dihydro-benzo[1,4]dioxin-2-yl)-[4-(4-fluoro-benzyl)-piperazin-1-yl]-methanone was prepared by a process similar to that described in Example 1. LC/MS [M+H]⁺: 391.2. ¹H-NMR (400 MHz, DMSO-d₆) δ (ppm): 7.27 (m, 2H), 2.00 (m, 2H), 6.89 (br s, 1H), 6.81 (m, 2H), 4.79 (m, 1H), 4.46 (dd, 1H), 4.29 (m, 1H), 3.71 (m, 2H), 3.55 (m, 2H), 3.48 (m, 2H), 2.43 (m, 4H). HPLC purity: 98.1%

### Example 5

### (6-Chloro-2,3-dihydro-benzo[1,4]dioxin-2-yl)-[4-(4-fluoro-benzyl)-[1,4]diazepan-1-yl]-methanone

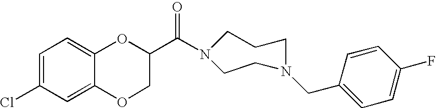

1-(4-Fluoro-benzyl)-[1,4]diazepane was prepared from 1-Boc-[1,4]diazapane using a procedure similar to that described for 1-(4-fluoro-benzyl)-piperazine in Example 4.

To a solution of 6-chloro-2,3-dihydro-benzo[1,4]dioxine-2-carboxylic acid (as described above in example 1) (97 mg, 0.47 mmol) DMF was added 1-(4-Fluoro-benzyl)-[1,4]diazepane (100 mg, 0.47 mmol), EDCI (116 mg, 0.61 mmol), HOBt (31 mg, 0.24 mmol) and DIPEA (0.23 ml, 1.4 mmol) at 5-10° C. and was stirred the reaction mixture at room temperature for 14-16 hours. The reaction mixture was diluted with water and extracted with ethyl acetate. The ethyl acetate solution was concentrated in vacuo and subjected to column chromatography (45% ethyl acetate-hexane) to afford 45 mg (24%) of (6-Chloro-2,3-dihydro-benzo[1,4]dioxin-2-yl)-[4-(4-fluoro-benzyl)-[1,4]diazepan-1-yl]-methanone. LC/MS [M+H]⁺: 405.0. ¹H-NMR (400 MHz, DMSO-d₆) δ (ppm): 7.37-6.88 (7 Ar H), 5.20 (m, 1H), 4.38 (m, 1H), 4.20 (m, 1H), 3.74-3.36 (m, 6H), 2.71 (m, 1H), 2.57 (m, 3H), 1.84 (m, 1H), 1.73 (m, 1H). HPLC purity: 94.3%

### Example 6

### (6,7-Dichloro-2,3-dihydro-benzo[1,4]dioxin-2-yl)-[(R)-4-(4-fluoro-benzyl)-2-methyl-piperazin-1-yl]-methanone

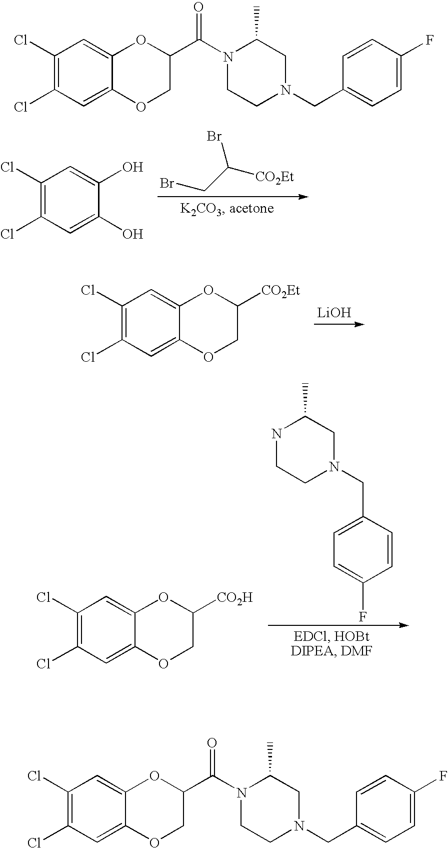

Step 1: 6,7-Dichloro-2,3-dihydro-benzo[1,4]dioxine-2-carboxylic acid ethyl ester

A mixture of 4,5-dichlorocatechol (250 mg, 1.4 mmol) was refluxed with ethyl-1,2-dibromopropionate (363 mg, 1.4 mmol) in acetone with potassium carbonate (290 mg, 2.1 mmol) for 9 h. The reaction mixture was concentrated and extracted with ethyl acetate. The ethyl acetate layer was concentrated and the crude product was subjected to column chromatography (silica gel, 5% ethyl acetate-hexane) to afford 310 mg (81%) of 6,7-Dichloro-2,3-dihydro-benzo[1,4]dioxine-2-carboxylic acid ethyl ester. LC/MS [M+H]⁺: 277.1. ¹H-NMR (400 MHz, CDCl3) δ (ppm): 7.09 (s, 1H), 6.96 (s, 1H), 4.81 (m, 1H), 4.42 (m, 1H), 4.28 (m, 1H), 4.25 (q, 2H), 1.28 (t, 3H).

Step 2: 6,7-Dichloro-2,3-dihydro-benzo[1,4]dioxine-2-carboxylic acid

To a solution of 6,7-Dichloro-2,3-dihydro-benzo[1,4]dioxine-2-carboxylic acid ethyl ester (310 mg, 1.12 mmol) in THF: H₂O (2:1) was added LiOH (61 mg, 1.45 mmol) and the resulting mixture was stirred for 3 hours. The reaction mixture was then concentrated and the crude mass was neutralized with 6(N) HCl solution and extracted with ethyl acetate. Concentration of the ethyl acetate layer afforded 210 mg (71.4%) of 6,7-Dichloro-2,3-dihydro-benzo[1,4]dioxine-2-carboxylic acid. ¹H-NMR (400 MHz, CDCl3) δ (ppm): 7.29-6.93 (2Ar H), 4.66 (m, 1H), 4.35 (m, 1H ), 4.12 (m, 1H).

Step 3: (6,7-Dichloro-2,3-dihydro-benzo[1,4]dioxin-2-yl)-[(R)-4-(4-fluoro-benzyl)-2-methyl-piperazin-1-yl]-methanone

To a solution of 6,7-Dichloro-2,3-dihydro-benzo[1,4]dioxine-2-carboxylic acid (100 mg, 0.48 mmol) in dichloromethane was added (R)-1-(4-fluoro-benzyl)-3-methyl-piperazine (119.6 mg, 0.48 mmol), EDCI (119.4 mg, 0.62 mmol), HOBt (32.4 mg, 0.24 mmol) and DIPEA (0.25 ml, 1.44 mmol) at 5-10° C. and the reaction mixture was stirred at room temperature for 14-16 hours. The mixture was diluted with water and extracted with ethyl acetate. The ethyl acetate layer was concentrated in vacuo and subjected to column chromatography (45% ethyl acetate-hexane) to afford 25 mg (11.8%) of (6,7-dichloro-2,3-dihydro-benzo[1,4]dioxin-2-yl)-[(R)-4-(4-fluoro-benzyl)-2-methyl-piperazin-1-yl]-methanone. LC/MS [M+H]⁺: 439.0. ¹H-NMR (400 MHz, DMSO-d₆) δ (ppm): 7.3-6.98 (m, 6H), 4.73 (m, 2H), 4.46 (m, 1H), 4.29 (m, 2H), 4.11 (m, 1H), 3.51 (m, 1H), 3.43 (m, 1H), 2.84 (m, 1H), 2.68 (m, 1H), 2.16 (m, 1H), 2.05 (m, 1H), 1.52 (m, 1H), 1.39 (d, 1H), 1.28 (m, 1H). HPLC purity: 94.9%

### Example 7

### (6,7-Dichloro-2,3-dihydro-benzo[1,4]dioxin-2-yl)-[4-(4-fluoro-benzyl)-[1,4]diazepan-1-yl]-methanone

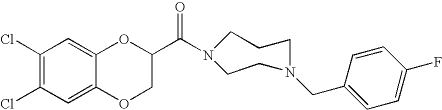

(6,7-Dichloro-2,3-dihydro-benzo[1,4]dioxin-2-yl)-[4-(4-fluoro-benzyl)-[1,4]diazepan-1-yl]-methanone was prepared by a process similar to that described in Example 6. LC/MS [M+H]⁺: 439.3. ¹H-NMR (400 MHz, DMSO-d₆) δ (ppm): 7.37-7.11 (m, 6H), 5.29 (m, 1H), 4.38 (m, 1H), 4.28 (m, 1H), 4.03-1.71 (m, 12H). HPLC purity: 89.6%

### Example 8

### (6-Chloro-3,4-dihydro-2H-benzo[1,4]oxazin-2-yl)-[(R)-4-(4-fluoro-benzyl)-2-methyl-piperazin-2-yl]-methanone

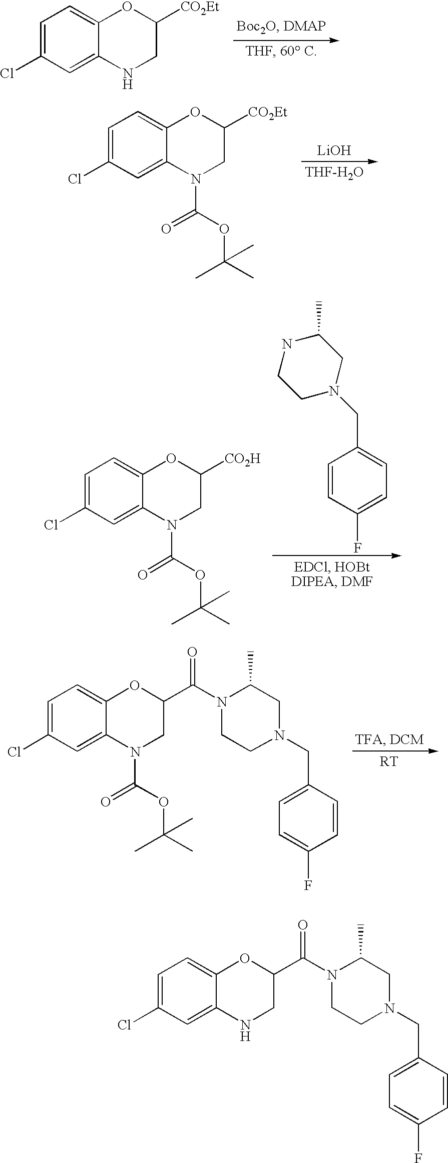

Step-1: 6-Chloro-2,3-dihydro-benzo[1,4]oxazine-2,4-dicarboxylic acid 4-tert-butyl ester 2-ethyl ester

To a solution of ethyl-6-chloro-3,4-dihydro-2H-benzo[1,4]oxazine-2-carboxylate (Key Organics, UK) (500 mg, 2.1 mmol) in THF was added DMAP (50 mg, 5.1 mmol) and di-tert-butyl dicarbonate (0.7 ml, 3.1 mmol). The solution was stirred for 18 h. The reaction mixture was concentrated. The crude mass was diluted with water and extracted with ethyl acetate. Concentration of ethyl acetate solution afforded 160 mg (100%) of 6-Chloro-2,3-dihydro-benzo[1,4]oxazine-2,4-dicarboxylic acid 4-tert-butyl ester 2-ethyl ester. LC/MS [M+H]⁺: ¹H-NMR (400 MHz, DMSO-d₆) δ (ppm): 7.71 (br s, 1H), 7.08-6.97 2 Ar H), 5.21 (t, J=3.1 Hz, 1H), 4.41 (m, 1H), 4.11 (m, 1H), 3.56 (dd, J=11.0, 2.9 Hz, 1H), 1.38 (s, 9H), 1.17 (t, J=7.0 Hz, 3H).

Step-2: 6-Chloro-2,3-dihydro-benzo[1,4]oxazine-2,4-dicarboxylic acid 4-tert-butyl ester

To a solution of ethyl6-Chloro-2,3-dihydro-benzo[1,4]oxazine-2,4-dicarboxylic acid 4-tert-butyl ester 2-ethyl ester (302 mg, 0.88 mmol) in THF: H₂O (2:1) was added lithium hydroxide (74.9 mg, 1.77 mmol). The reaction mixture was stirred for 5-6 h then concentrated. The crude mass was neutralized with 50% aqueous acetic acid and extracted with ethyl acetate. Concentration of the ethyl acetate layer afforded 270 mg (97%) of 6-Chloro-2,3-dihydro-benzo[1,4]oxazine-2,4-dicarboxylic acid 4-tert-butyl ester. GCMS [m/z-Boc]: 213. ¹H-NMR (400 MHz, DMSO-d₆) δ (ppm):13.40 (br s, 1H), 7.72-6.95 (3 Ar H), 5.06 (br, s, 1H), 4.40 (dd, J=11.2, 2.6 Hz, 1H), 3.55 (dd, J=10.8, 3.0 Hz, 1H), 1.38 (s, 9H)

Step-3: 6-Chloro-2-[(R)-4-(4-fluoro-benzyl)-2-methyl-piperazine-1-carbonyl]-2,3-dihydro-benzo[1,4]oxazine-4-carboxylic acid tert-butyl ester

To a solution of 6-chloro-2,3-dihydro-benzo[1,4]oxazine-2,4-dicarboxylic acid 4-tert-butyl ester (415 mg, 1.3 mmol) in DMF was added (R)-1-(4-fluoro-benzyl)-3-methyl-piperazine (257 mg, 1.3 mmol)), EDCI (330 mg, 1.7 mmol), HOBt (89 mg, 0.7 mmol) and DIPEA (0.7 ml, 3.9 mmol) at 5-10° C. The reaction mixture was stirred at room temperature for 14-16 then diluted with water and extracted with ethyl acetate. The ethyl acetate layer was concentrated in vacuo and subjected to column chromatography (Si-gel, 45% ethyl acetate-hexane) to afford 197 mg (30%) of 6-chloro-2-[(R)-4-(4-fluoro-benzyl)-2-methyl-piperazine-1-carbonyl]-2,3-dihydro-benzo[1,4]oxazine-4-carboxylic acid tert-butyl ester. LC/MS [M+H]⁺: 504.4

Step-4: (6-Chloro-3,4-dihydro-2H-benzo[1,4]oxazin-2-yl)-[(R)-4-(4-fluoro-benzyl)-2methyl-piperazin-1-yl]-methanone

To a solution of 6-chloro-2-[(R)-4-(4-fluoro-benzyl)-2-methyl-piperazine-1-carbonyl]-2,3-dihydro-benzo[1,4]oxazine-4-carboxylic acid tert-butyl ester (190 mg, 0.37 mmol) in DCM was added trifluoroacetic acid (0.3 ml, 3.9 mmol). The reaction mixture was stirred for about 5 hours then concentrated and washed with dry ether. The TFA salt was diluted with a minimum volume of water and neutralized with aq. NaOH solution at 5-10° C. Extraction of the neutralized solution followed by concentration afforded 114 mg (75%) of (6-chloro-3,4-dihydro-2H-benzo[1,4]oxazin-2-yl)-[(R)-4-(4-fluoro-benzyl)-2-methyl-piperazin-1-yl]-methanone. LC/MS [M+H]⁺: 404.2 ¹H-NMR VT 90° C. (400 MHz, DMSO-d₆) δ (ppm): 7.35 (m, 2H), 7.12 (m, 2H), 6.67 (m, 1H), 6.62 (d, J=2.4 Hz, 1H), 6.46 (m, 1H), 5.84 (br s, 1H), 4.80 (m, 1H), 4.45 (m, 1H), 3.96 (m, 1H), 3.53-3.21 (m, 5H), 2.80 (m, 1H), 2.65 (m, 1H), 2.16 (m, 1H), 2.03 (m, 1H), 1.25 (d, J=6.7 Hz, 3H), HPLC purity: 99.7%.

### Example 9

### (6-Chloro-3,4-dihydro-2H-benzo[1,4]oxazin-2-yl)-[4-(4-fluoro-benzyl)-piperazin-1-yl]-methanone

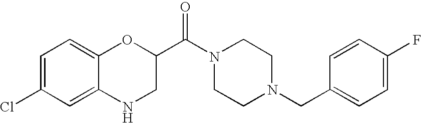

(6-Chloro-3,4-dihydro-2H-benzo[1,4]oxazin-2-yl)-[4-(4-fluoro-benzyl)-piperazin-1-yl]-methanone was prepared by a process similar to that described in Example 8. LC/MS [M+H]⁺: 390.3. ¹H-NMR (400 MHz, DMSO-d₆) δ (ppm): 7.35 (m, 2H), 7.15 (t, J=8.8 Hz, 2H), 6.67 (d, J=8.4 Hz, 1H), 6.57 (d, J=2.4 Hz, 1H), 6.46 (dd, J=6.0, 2.4 Hz, 1H), 6.17 (s, 1H), 4.89 (m, 1H), 3.57-3.38 (br m, 7H), 2.66 (m, 1H), 2.40-2.32 (m, 4H). HPLC purity: 80.9%

### Example 10

### (6-Chloro-3,4-dihydro-2H-benzo[1,4]oxazin-2-yl)-[4-(4-fluoro-benzyl)-[1,4]diazepan-1-yl]-methanone

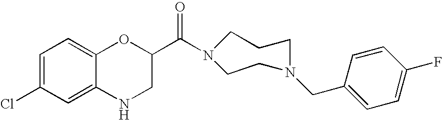

(6-Chloro-3,4-dihydro-2H-benzo[1,4]oxazin-2-yl)-[4-(4-fluoro-benzyl)-[1,4]diazepan-1yl]-methanone was prepared by a process similar to that described in Example 8. LC/MS [M+H]⁺: 404.3. ¹H-NMR (400 MHz, DMSO-d₆) δ (ppm): 7.34 (m, 2H), 7.13 (m, 2H), 6.70-6.58 (m, 2H), 6.45 (m, 1H), 6.18 (s, 1H), 4.83 (m, 1H), 3.67-1.73 (m, 14H). HPLC purity: 81.8%

### Example 11

### 6-Chloro-2-[4-(4-fluoro-benzyl)-[1,4]diazepane-1-carbonyl]-2,3-dihydro-benzo[1,4]oxazine-4-carboxylic acid amide

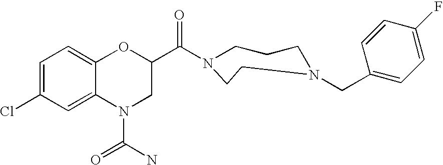

To a solution of (6-chloro-3,4-dihydro-2H-benzo[1,4]oxazin-2-yl)-[4-(4-fluoro-benzyl)-[1,4]diazepan-1-yl]-methanone (47 mg, 0.17 mmol) in THF was added triethylamine (0.03 ml, 0.23 mmol) and ethyl chloroformate (0.01 ml, 0.16 mmol) under ice-cold condition. The reaction mixture was stirred overnight, then concentrated and extracted with ethyl acetate. The ethyl acetate layer was concentrated. The concentrated mass was dissolved in ethanol and condensed with NH₃ at 0° C. in a sealed tube and the solution was heated at 70° C. overnight. The solution was concentrated in vacuo and subjected to column chromatography (Si-gel, 2% MeOH-DCM) to obtain 11 mg (25%) 6-chloro-2-[4-(4-fluoro-benzyl)-[1,4]diazepane-1-carbonyl]-2,3-dihydro-benzo[1,4]oxazine-4-carboxylic acid amide. LC/MS [M+H]: 446. HPLC purity: 83.21%

### Example 12

### (6-Chloro-4-methyl-3,4-dihydro-2H-benzo[1,4]oxazin-2-yl)-[(R)-4-(4-fluoro-benzyl)-2-methyl-piperazin-1-yl]-methanone

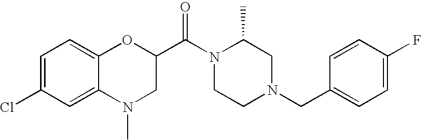

To a solution of 6-chloro-4-methyl-3,4-dihydro-2H-benzo[1,4]oxazine-2-carboxylic acid (prepared from 6-Chloro-2,3-dihydro-benzo[1,4]oxazine-2,4-dicarboxylic acid 4-tert-butyl ester 2-ethyl ester with cesium carbonate and methyl iodide followed by hydrolysis with lithium hydroxide) (100 mg, 0.44 mmol) in DMF was added 1-(4-fluoro-benzyl)-[1,4]diazepane (100 mg, 0.44 mmol), EDCI (109 mg, 0.57 mmol), HOBt (30 mg, 0.22 mmol) and DIPEA (0.22 ml, 1.32 mmol) at 5-10° C. The reaction mixture was stirred at room temperature for 15 hours then diluted with water and extracted with ethyl acetate. The ethyl acetate layer was concentrated in vacuo and subjected to column chromatography (Si-gel, 3% MeOH-DCM) to afford 50 mg (27%) of (6-chloro-4-methyl-3,4-dihydro-2H-benzo[1,4]oxazin-2-yl)-[(R)-4-(4-fluoro-benzyl)-2-methyl-piperazin-1-yl]-methanone. LC/MS [M+H]⁺: 418.1. ¹H-NMR (400 MHz, DMSO-d₆) δ (ppm): 7.35 (m, 2H), 7.15 (m, 2H), 6.67 (m, 2H), 6.56 (br s, 1H), 5.75 (s, 1H), 5.05 (M, 1H), 4.47-3.30 (m, 7H), 2.89 (s, 3H), 2.78-0.85 (m, 8H). HPLC purity: 99.4%

### Example 13

### (6-Chloro-4-methyl-3,4-dihydro-2H-benzo[1,4]oxazin-2-yl)-[4-(4-fluoro-benzyl)-[1,4]diazepan-1-yl]-methanone

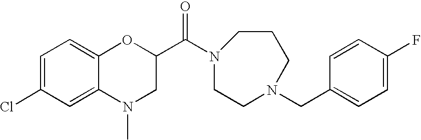

To a solution of 6-chloro-4-methyl-3,4-dihydro-2H-benzo[1,4]oxazine-2-carboxylic acid (prepared from 6-Chloro-2,3-dihydro-benzo[1,4]oxazine-2,4-dicarboxylic acid 4-tert-butyl ester 2-ethyl ester with cesium carbonate and methyl iodide followed by hydrolysis with lithium hydroxide) (100 mg, 0.44 mmol) in DMF was added 1-(4-fluoro-benzyl)-[1,4]diazepane (100 mg, 0.44 mmol), EDCI (109 mg, 0.57 mmol), HOBt (30 mg, 0.22 mmol) and DIPEA (0.22 ml, 1.32 mmol) at 5-10° C. The reaction mixture was stirred at room temperature for 15 hours then diluted with water and extracted with ethyl acetate. The ethyl acetate layer was concentrated in vacuo and subjected to column chromatography (Si-gel, 3% MeOH-DCM) to afford 50 mg (27%) of (6-chloro-4-methyl-3,4-dihydro-2H-benzo[1,4]oxazin-2-yl)-[4-4-fluoro-benzyl)-[1,4]diazepan-1-yl]-methanone. LC/MS [M+H]⁺: 418.1. ¹H-NMR (400 MHz, DMSO-d₆) δ (ppm): 7.34 (m, 2H), 7.13 (m, 2H), 6.67 (m, 2H), 6.55 (m, 1H), 5.05 (m, 1H), 3.67 (m, 1H), 3.61 (m, 3H), 3.48 (m, 1H), 3.44 (m, 1H), 3.40 (m, 1H), 2.85 (s, 3H), 2.74 (m, 2H), 2.71 (m, 4H), 1.83 (m, 1H), 1.73 (m, 1H). HPLC purity: 98.1%

### Example 14

### 6-Chloro-2-[(R)-4-(4-fluoro-benzyl)-2-methyl-piperazine-1-carbonyl]-4H-benzo[1,4]oxazin-3-one

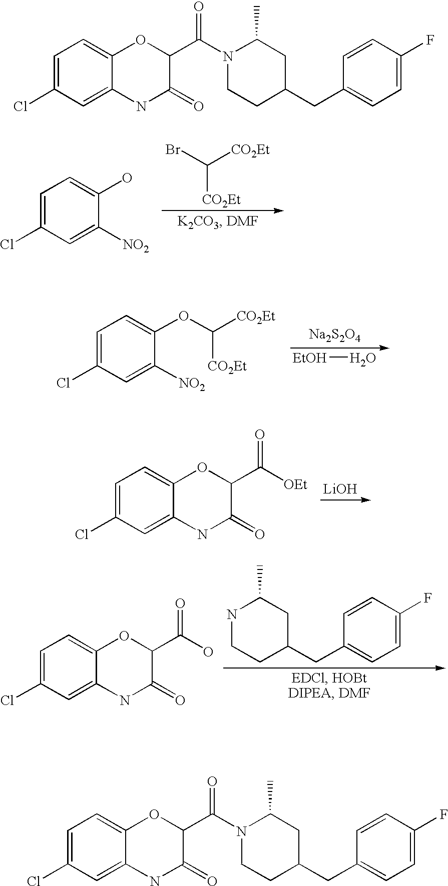

Step-1: 2-(4-Chloro-2-nitro-phenoxy)-malonic acid diethyl ester

4-Chloro-2-nitrophenol (1 g, 5.76 mmol) was dissolved in a minimum volume of DMF, and then anhydrous K₂CO₃ 1.6 g (11.5 mmol) and diethyl-2-bromomalonate 1.08 ml (6.34 mmol) were added to the solution. The resulting mixture was stirred at room temperature overnight. The mixture was diluted with ice-water and extracted with ethyl acetate. The organic layer was washed successively with water and brine then dried over Na₂SO₄. The organic layer was concentrated under reduced pressure and the residue was purified by flash chromatography (silica-gel, 2% EtOAc-hexane) to afford 600 mg (32%) of 2-(4-Chloro-2-nitro-phenoxy)-malonic acid diethyl ester. LC/MS [M+H]⁺: 332.1. ¹ H-NMR (400 MHz, DMSO-d₆) δ (ppm): 8.08 (s, 1H), 7.74 (m, 1H), 7.37 (d, 1H), 4.21 (m, 4H), 5.99 (s, 1H), 1.20 (t, 6H).

Step-2: 6-Chloro-3-oxo-3,4-dihydro-2H-benzo[1,4]oxazine-2-carboxylic acid ethyl ester

2-(4-Chloro-2-nitro-phenoxy)-malonic acid diethyl ester (4.8 g, 14.5 mmol) was dissolved in EtOH: H₂O (4:5) and then Na₂S₂O₄. H₂O (17 g, 88.3 mmol) was added. The reaction mixture was stirred at 60° C. for 5 h. The mixtures was concentrated under reduced pressure and diluted with water. The reaction mass was then extracted with ethyl acetate. The organic layer was washed with water and brine, dried over Na₂SO₄ and concentrated under reduce pressure to afford 3.6 g (97.3%) of 6-Chloro-3-oxo-3,4-dihydro-2H-benzo[1,4]oxazine-2-carboxylic acid ethyl ester. LC/MS [M+H]⁺: 256.2. ¹H-NMR (400 MHz, DMSO-d₆) δ (ppm): 11.11 (s, 1H), 7.07 (d, 1H0, 7.01 (dd, 1H), 6.90 (d, 1H), 5.48 (s, 1H), 4.16 (m, 2H), 1.15 (t, 3H)

Step-3: 6-Chloro-3-oxo-3,4-dihydro-2H-benzo[1,4]oxazine-2-carboxylic acid

To a solution of 6-Chloro-3-oxo-3,4-dihydro-2H-benzo[1,4]oxazine-2-carboxylic acid ethyl ester (200 mg, 0.78 mmol) in THF: H₂O (2:1) was added lithium hydroxide (82 mg, 1.95 mmol). The reaction mixture was stirred for 5-6 h then concentrated. The crude mass was neutralized with 50% aqueous acetic acid and extracted with ethyl acetate. Concentration of the ethyl acetate layer afforded 150 mg (84.2%) of 6-chloro-3-oxo-3,4-dihydro-2H-benzo[1,4]oxazine-2-carboxylic acid. LC/MS [M+H]⁺: 227.9. ¹H-NMR (400 MHz, DMSO-d₆) δ (ppm): 13.69 (br s, 1H), 11.01 (s, 1H), 7.04 (d, 1H), 6.98 (d, 1H), 6.88 (br s, 1H), 5.30 (s, 1H).

Step-4: 6-Chloro-2-[(R)-4-(4-fluoro-benzyl)-2-methyl-piperazine-1-carbonyl]-4H-benzo[1,4]oxazin-3-one

To a solution of 6-chloro-3,4-dihydro-2H-benzo[1,4]oxazine-2-carboxylic acid (100 mg, 0.44 mmol) in DMF was added (R)-1-(4-fluoro-benzyl)-3-methyl-piperazine (92 mg, 0.44 mmol), EDCI (109 mg, 0.57 mmol), HOBt (29 mg, 0.22 mmol) and DIPEA (0.2 ml, 1.3 mmol) at 5-10° C. The reaction mixture was stirred at room temperature for 14-16 hours then diluted with water and extracted with ethyl acetate. The ethyl acetate layer was concentrated in vacuo and subjected to column chromatography (45% ethyl acetate-hexane) to afford 50 (27.2%) of 6-chloro-2-[(R)-4-(4-fluoro-benzyl)-2-methyl-piperazine-1-carbonyl]-4H-benzo[1,4]oxazin-3-one. LC/MS [M+H]⁺: 417.9. ¹H-NMR (400 MHz, CDCl₃) δ (ppm): 7.88 (m, 1H), 7.27 (m, 2H), 7.00 (m, 2H), 6.93 (m, 2H), 6.78 (m, 1H), 5.45 (m, 1H), 5.39 (m, 1H), 4.56-2.0 (m, 7H), 1.43 (m, 1H), 1.18 (m, 3H). HPLC purity: 95.1%

### Example 15

### 1-{6-Chloro-2-[4-(4-fluoro-benzyl)-piperazine-1-carbonyl]-2,3-dihydro-benzo[1,4]oxazin-4-yl}-ethanone

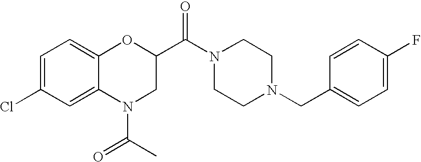

(6-Chloro-3,4-dihydro-2H-benzo[1,4]oxazin-2-yl)-[4-(4-fluoro-benzyl)-piperazin-1-yl]-methanone (75 mg, 0.19 mmol) was dissolved in dichloromethane. Acetyl chloride (0.02 ml, 0.23 mmol) followed triethylamine (0.05 ml, 0.38 mmol) were then added at 0° C. The reaction mixture was stirred at room temperature overnight. The mixture was diluted with water and extracted with ethyl acetate. The organic layer was dried over sodium sulfate and concentrated. Purification of crude reaction mass over silica-gel afforded 35 mg (42.7%) of 1-{6-chloro-2-[4-(4-fluoro-benzyl)-piperazine-1-carbonyl]-2,3-dihydro-benzo[1,4]oxazin-4-yl}-ethanone. LC/MS [M+H]⁺: 432.2. ¹H-NMR (400 MHz, DMSO-d₆) δ (ppm): 7.6 (m, 1H), 7.35 (br s, 2H), 7.12 (m, 3H), 6.95 (d, 1H), 5.39 (br s, 1H0, 4.35 (m, 2H), 3.49 (m, 4H), 2.90 (br s, 1H), 2.32 (s, 3H), 1.50 (m, 1H), 1.23 (m, 3H). HPLC purity: 98.3%

### Example 16

### (6-Chloro-4-methanesulfonyl-3,4-dihydro-2H-benzo[1,4]oxazin-2-yl)-[4-(4-fluoro-benzyl)-piperazin-1-methanone

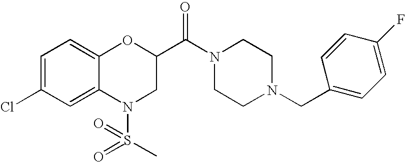

(6-Chloro-3,4-dihydro-2H-benzo[1,4]oxazin-2-yl)-[4-(4-fluoro-benzyl)-piperazin-1-yl]-methanone (70 mg, 0.18 mmol) was dissolved in dichloromethane. Methane sulfonyl chloride (0.02 ml, 0.22 mmol) followed triethylamine (0.05 ml, 0.36 mmol) were then added at 0° C. The reaction mixture was stirred at room temperature overnight. The mixture was diluted with water and extracted with ethyl acetate. The organic layer was dried over sodium sulfate and concentrated. Purification of crude reaction mass over silica-gel afforded 50 mg (59.4%) of (6-chloro-4-methanesulfonyl-3,4-dihydro-2H-1-benzo[1,4]oxazin-2-yl)-[4-(4-fluoro-benzyl)-piperazin-1-yl]-methanone. LC/MS [M+H]⁺: 468.1. ¹H-NMR (400 MHz, DMSO-d₆) δ (ppm): 7.50-7.02 (m, 7H), 5.21 (br s, 1H), 4.40 (m, 4H), 4.05 (m, 2H), 3.69 (m, 1H), 3.01 (br s, 2H), 2.32 (m, 3H), 1.23 (s, 2H), 0.85 (m, 1H). HPLC purity: 98.8%

### Example 17

### (6-Chloro-3,4-dihydro-2H-benzo[1,4]oxazin-2-yl)-[7-(4-fluoro-benzyl)-[1,4]oxazepan-4yl]methanone

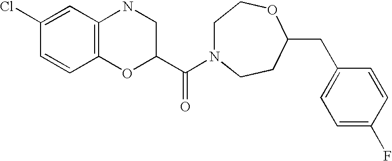

Preparation of 7-(4-fluoro-benzyl)[1,4]oxazepane

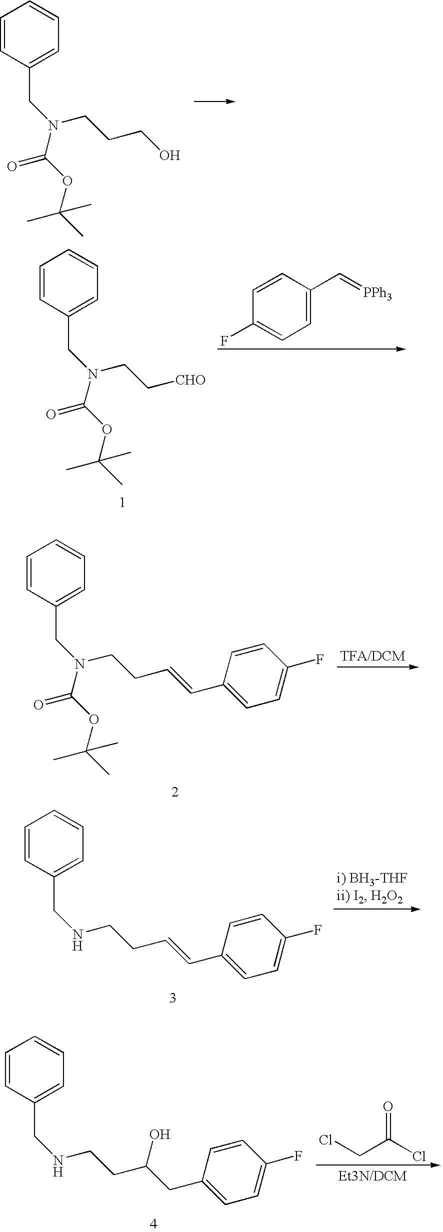

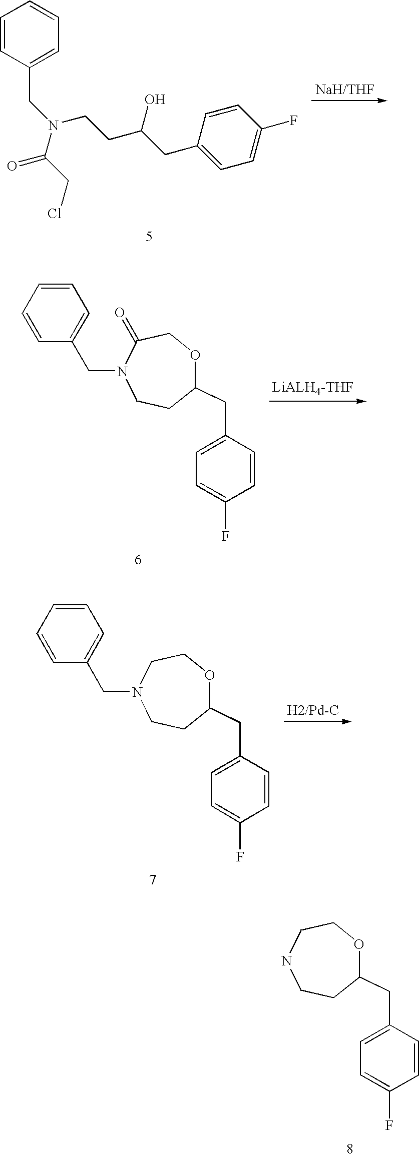

Step-1: (3-Hydroxy-propyl)-[(E)-((Z)-2-propenyl)-penta-2,4-dienyl]-carbamic acid tert-butyl ester

To a solution of 3-benzylamino-propan-1-ol (10 g, 0.06 mol) in DCM was added 1(N) NaOH and Boc-anhydride (13.5 ml, 0.06 mol) at 0° C. The solution was stirred overnight at room temperature. The organic layer was separated, washed with water and concentrated under reduced pressure. The crude product was subjected to column chromatography (Si-gel, 15% EtOAc-hexane) to afford 15.1 g of (3-Hydroxy-propyl)-[(E)-((Z)-2-propenyl)-penta-2,4-dienyl]-carbamic acid tert-butyl ester in 99% yield. LC/MS [M+H]⁺: 266.4. ¹H-NMR (400 MHz, CDCl₃) δ (ppm): 7.33-7.21 (m, 5-ArH), 4.37 (s, 2H), 3.71 (s, 1H), 3.55 (br s, 3H), 3.37 (br. s, 3H), 1.45 (s, 9H).

Step-2: (3-propanyl)-[(E)-((Z)-2-propenyl)-penta-2,4-dienyl]-carbamic acid tert-butyl ester

To a solution of DMSO (13.5 ml, 0.19 mol) in anhydrous dichloromethane was added drop wise a solution of oxalyl chloride (7.2 ml, 0.08 mol) in anhydrous dichloromethane under a nitrogen atmosphere at −78° C. The resulting mixture was stirred for 15 minutes. A solution of (3-hydroxypropyl)-[(E)-((Z)-2-propenyl)-penta-2,4-dienyl]-carbamic acid tert-butyl ester (20.2 g, 0.07 mol) in dichloromethane (85 ml) was then added dropwise to the reaction mixture over a period of 10 min and stirring was continued for 2 h. To the solution was added triethyl amine (53 ml, 0.38 mol) and stirring was continued for 5 min at −78° C. followed by stirring at RT 30 min. The reaction mixture was diluted with water and extracted with dichloromethane. The combined organic layer was washed with brine, water, 5% sodium bicarbonate and dried over anhydrous sodium sulphate. Concentration under reduced pressure afforded 18.1 g of (3-propanyl)-[(E)-((Z)-2-propenyl)-penta-2,4-dienyl]-carbamic acid tert-butyl ester in 92% yield. This was used in the next step without further purification. ¹H-NMR (400 MHz, DMSO-d₆) δ (ppm): 9.63 (s, 1H), 7.37-7.22 (m, 5H), 4.37 (s, 2H), 3.41 (br. s, 2H), 2.60 (t, J=1.0 Hz, 2H), 1.38 (s, 9H).

Step-3: Benzyl-[(E)-4-(4-fluoro-phenyl)-but-3-enyl]-carbamic acid tert-butyl ester

To a solution of (4-fluorobenzyl)triphenylphosphonium bromide (28 g, 0.075 mol) in THF was added t-BuOK (8.5 g, 0.075 mol) portion wise at 0° C. and stirred for 1 h at room temperature. A solution of (3-propanyl)-[(E)-((Z)-2-propenyl)-penta-2,4-dienyl]-carbamic acid tert-butyl ester (18 g, 0.68 mol) in THF was added drop wise to the generated ylide solution at 0° C. The reaction mixture was stirred for 5 h at room temperature then extracted with ethyl acetate. Concentration of the ethyl acetate solution and purification of the crude product by column chromatography (Si-gel, 3% ethyl acetate-hexane) afforded 10 g (62%) of benzyl-[(E)-4-(4-fluoro-phenyl)-but-3-enyl]-carbamic acid tert-butyl ester. LC/MS [M+H]⁺: 356.2. ¹H-NMR (400 MHz, CDCl₃) δ (ppm): 7.33-6.93 (m, 9 ArH), 6.45 (d, J=11.6 Hz, 1H), 6.02 (br s, 1H), 4.43 (br s, 2H) 3.29 (br, 2H), 2.42 (br, 2H), 1.44 (s, 9H).

Step-4: Benzyl-[(E)-4-(4-fluoro-phenyl)-but-3-enyl]-amine

To a solution of benzyl-[(E)-4-(4-fluoro-phenyl)-but-3-enyl]-carbamic acid tert-butyl ester (10 g, 0.028 mol) in DCM was added trifluoroacetic acid (21.5 ml, 0.28 mol)) and stirred the solution for about 2 h. The solution was concentrated and washed with dry ether. The resulting TFA salt was diluted with a minimum volume of water and neutralized with aq. NaOH solution at 5-10° C. Extraction of the aqueous solution with ethyl acetate followed by concentration of the organic layer afforded 6.8 g (95%) of benzyl-[(E)-4-(4-fluoro-phenyl)-but-3-enyl]-amine. LC/MS [M+H]⁺: 256. ¹H-NMR (400 MHz, DMSO-d₆) δ (ppm): 8.95 (s, 1H), 7.52-7.14 (m, 8 ArH), 6.53 (t, J=16.2 Hz, 1H), 6.21 (m, 1H), 4.18 (d, J=13.4 Hz, 2H), 3.05 (m, 2H), 2.65 (m, 1H), 2.54 (m, 1H).

Step-5: 4-Benzylamino-1-(4-fluoro-phenyl)-butan-2-ol

Benzyl-[(E)-4-(4-fluoro-phenyl)-but-3-enyl]-amine (14g, 0.027 mol) was dissolved in 100 ml THF and cooled to 0° C. BH₃-THF (1.5 M, 18.2 ml) was slowly added dropwise and the solution was stirred for 45 minutes. After warming the reaction mixture to room temperature, the reaction mixture was concentrated under reduced pressure.

Iodine (2 g, 0.013 mol) dissolved in dichloromethane was added dropwise to a solution of crude amine-borane complex (14 g, 0.26 mol) at room temperature. After 30 minutes, the solvent was evaporated leaving the crude hydroborated product. Oxidation was carried out by adding MeOH (200 ml) followed by 20% NaOH (70 ml) and 30% H₂O₂ (63 ml). The white suspension was stirred overnight at room temperature. The methanolic reaction mixture was concentrated and extracted with dichloromethane. The combined organic layer was dried over sodium sulfate and the solvent was removed under reduced pressure. The crude reaction mass was purified through Si-gel (3% meOH-DCM) to afford 4 g (54.2%) of 4-benzylamino-1-(4-fluoro-phenyl)-butan-2-ol. LC/MS [M+H]⁺: 274.2

Step-6: N-Benzyl-2-chloro-N-[4-(4fluoro-phenyl)-3-hydroxy-butyl]-acetamide

To a solution of 4-benzylamino-1-(4-fluoro-phenyl)-butan-2-ol (2 g, 7.3 mmol) in dichloromethane was added triethylamine (1.1 ml, 8.03 mmol). Chloroacetyl chloride (0.58 ml, 7.3 mmol) was then added at 0° C. and the mixture stirred for 2 hours at room temperature. The mixture was then concentrated, diluted with water and extracted with ethyl acetate. The organic layer was concentrated to afford 2.78 g N-benzyl-2-chloro-N-[4-(4-fluoro-phenyl)-3-hydroxy-butyl]-acetamide. LC/MS [M+H]⁺: 350. ¹H-NMR (400 MHz, DMSO-d₆) δ (ppm):7.38-7.04 (m, 9H), 4.74 (d, J=6.1 Hz, 1H), 4.62-4.35 (m, 5H), 3.57 (br s. 1H), 3.27 (m, 1H), 2.59 (d, J=6.1 Hz, 2H), 1.72 (m, 1H).

Step-7: 4-Benzyl-7-(4-fluoro-benzyl)-[1,4]oxazepan-3-one

To a solution of N-Benzyl-2-chloro-N-[4-(4-fluoro-phenyl)-3-hydroxy-butyl]-acetamide (2.9 g, 8.3 mmol) in THF was added sodium hydride (0.26 g, 10.7 mmol) portion wise at 0° C. The resulting mixture was stirred at room temperature. Excess NaH was quenched with saturated aqueous solution of ammonium chloride at 0° C. and with ethyl acetate. The organic layer was concentrated to afford 2.65 g of 4-benzyl-7-(4-fluoro-benzyl)-[1,4]oxazepan-3-one. LC/MS [M+H]⁺: 314. ¹H-NMR (400 MHz, CDCl₃) δ (ppm): 7.34-6.99 (m, 9H), 4.90-4.26 (m, 5H), 3.39 (d, J=13.5 Hz, 1H), 2.06 (m, 1H), 1.74 (m, 1H).

Step-8: 4-Benzyl-7-(4-fluoro-benzyl)[1,4]oxazepane

To a solution of 4-benzyl-7-(4-fluoro-benzyl)-[1,4]oxazepan-3-one (2.65g, 8.55 mmol) in THF was added LiAlH₄ (0.65 g, 17.1 mmol) at 0° C. The resulting mixture was heated to reflux for 2 houra. Excess LiALH₄ was quenched with saturated aqueous sodium sulfate solution and with ethyl acetate. The organic layer was concentrated and purified through Si-gel (10% ethyl acetate-hexane) to afford 1.3 g (50.3%) of 4-benzyl-7-(4-fluoro-benzyl)-[1,4]oxazepane. LC/MS [M+H]⁺: 300.2. ¹H-NMR (400 MHz, DMSO-d₆) δ (ppm): 7.32-7.05 (m, 9H), 3.88-3.70 (m, 2H), 3.57 (s, 2H), 3.44 (m, 1H), 2.722.52 (m, 5H), 1.81 (m, 1H), 1.68 (m, 1H)

Step-9: 7-(4-Fluoro-benzyl)-[1,4]oxazepane

4-benzyl-7-(4-fluoro-benzyl)-[1,4]oxazepane (1.3 g, 4.3 mmol) dissolved in ethanol was hydrogenated with 10% Pd-C (140 mg) for 12 hour. The catalyst was removed by filtration through a celite bed. Concentration of the ethanol solution afforded 850 mg (93%) of 7-(4-fluoro-benzyl)-[1,4]oxazepane. LC/MS [M+H]⁺: 210.4. ¹H-NMR (400 MHz, DMSO-d₆) δ (ppm): 7.16-6.92 (m, 4 ArH), 3.84 (m, 2H), 3.48 (m, 1H), 2.92 (m, 3H), 2.87 (m, 1H), 2.65 (m, 1H), 1.89 (m, 1 h, 1.62 (m, 1H).

(6-Chloro-3,4-dihydro-2H-benzo[1,4]oxazin-2-yl)-[7-(4-fluoro-benzyl)-[1,4]oxazepan-4-yl]-methanone was prepared by coupling 7-(4-Fluoro-benzyl)-[1,4]oxazepane with 6-Chloro-2,3-dihydro-benzo[1,4]oxazine-2,4-dicarboxylic acid 4-tert-butyl ester followed by deboc with TFA using the procedure described in Example 8. LC/MS [M+H]⁺: 405.4. ¹H-NMR (400 MHz, DMSO-d₆) δ (ppm): 7.16-6.34 (m, 7H), 4.74 (m, 1H), 4.72-3.23 (m, 10H), 2.80-2.66 (m, 2H), 1.72 (m, 2H). HPLC purity: 99.6%

### Example 18

### 1-{6-Chloro-2-[4-(4-fluoro-benzyl)-piperazine-1-carbonyl]-2,3-dihydro-benzo[1,4]oxazin-4-yl}-2-dimethylamino-ethanone

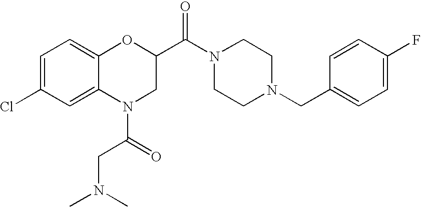

(6-chloro-3,4-dihydro-2H-benzo[1,4]oxazin-2-yl)-[4-(4-fluoro-benzyl)-piperazin-1-yl]-methanone (200 mg, 0.51 mmol) was dissolved in dichloromethane. Chloroacetyl chloride (0.04 ml, 0.51 mmol) followed by triethylamine (0.08 ml, 0.56 mmol) were then added at 0° C. The reaction mixture was stirred at room temperature overnight. The mixture was diluted with water and extracted with ethyl acetate. The organic layer was dried over sodium sulfate and concentrated to afford 240 mg of crude product. 120 mg of the crude product in Me₂NH-THF was heated at 70° C. in a sealed tube overnight. The reaction mixture was concentrated to afford 85 mg (68.8%) of 1-{6-chloro-2-[4-(4-fluoro-benzyl)-piperazine-1-carbonyl]-2,3-dihydro-benzo[1,4]oxazin-4-yl}-2-dimethylamino-ethanone. LC/MS [M+H]⁺: 475.1. ¹H-NMR (400 MHz, DMSO-d₆) δ (ppm): 8.44-6.86 (m, 7H), 5.36 (br s, 1H), 4.01 (br s, 1H), 3.62 (br s, 1H), 3.49 (m, 4H), 2.96 (d, 1H), 2.82 (d, 1H), 2.58 (m, 3H), 2.36 (m, 2H), 2.24 (s, 6H), 1.35 (m, 1H), 1.16 (m, 1H). HPLC purity: 95.1%

### Example 19

### (6-Chloro-4-methyl-3,4-dihydro-2H-benzo[1,4]oxazin-2-yl)-[4-(4-fluoro-benzyl)-piperazin-1-yl]-methanone

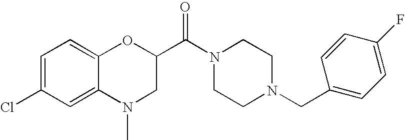

(6-chloro-4-methyl-3,4-dihydro-2H-benzo[1,4]oxazin-2-yl)-[4-(4-fluoro-benzyl)-piperazin-1-yl]-methanone was prepared using a process similar to that described in Example 12. LC/MS [M+H]⁺: 404.0. ¹H-NMR (400 MHz, CDCl₃) δ (ppm): 8.00-6.55 (m, 7H), 4.82 (m, 1H), 3.72 (m, 2H), 3.57-3.36 (m, 6H), 2.89 (s, 3H), 2.48-2.40 (m, 4H). HPLC purity: 90.1%

### Example 20

### 1-{6-Chloro-2-[4-(4-fluoro-benzyl)-piperazine-1-carbonyl]-2,3-dihydro-benzo[1,4]oxazin-4-yl}-2-methylamino-ethanone

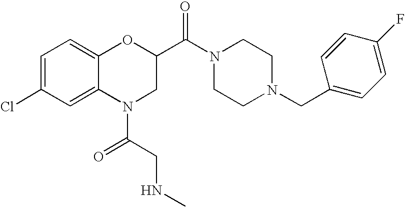

1-{6-Chloro-2-[4-(4-fluoro-benzyl)-piperazine-1-carbonyl]-2,3-dihydro-benzo[1,4]oxazin-4-yl}-2-methylamino-ethanone was prepared through aminolysis of the ester with a THF solution methylamine by a process similar to that described above in Example 18. LC/MS [M+H]⁺: 461.2.

¹H-NMR (400 MHz, DMSO-d₆) δ (ppm): 8.34-7.00 (m, 7H), 5.39 (br s, 1H), 4.27 (m, 2H), 3.79-3.31 (m, 7H), 2.66 (m, 1H), 2.53-0.85 (7H). HPLC purity: 91.1%

### Example 21

### (6-Chloro-3,4-dihydro-2H-benzo[1,4]oxazin-2-yl)-[4-(4-fluoro-benzoyl)-piperidin-1-yl]-methanone

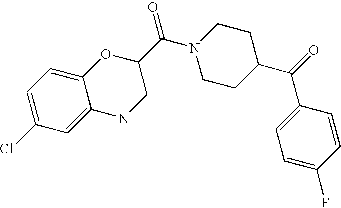

(6-Chloro-3,4-dihydro-2H-benzo[1,4]oxazin-2-yl)-[4-(4-fluoro-benzoyl)-piperidin-1-yl]-methanone was prepared from 4-fluorobenzoyl piperidine (Alfa Aesar, USA) by the amide coupling method described above in Example 8. LC/MS [M+H]⁺: 403.0. ¹H-NMR (400 MHz, DMSO-d₆) δ (ppm): 8.10 (br s, 2H), 7.37 (t, 2H), 6.68 (br s, 1H), 6.58s, 1H), 6.44 (d, 1H), 4.91 (br s, 1H), 4.35 (t, 1H), 4.03 (d, 1H), 3.75 (m, 1H), 3.48 (m, 2H), 3.31 (m, 2H), 2.85 (m, 2H), 1.99 (d, 2H), 1. 58 (m, 2H). HPLC purity: 92.2%

### Example 22

### 6-Chloro-3,4-dihydro-2H-benzo[1,4]oxazine-2-carboxylic acid [1-(4-fluoro-benzyl)-piperidin-4-yl]-amide

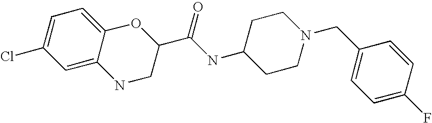

Preparation of 1-(4-fluoro-benzyl)-piperidin-4-ylamine

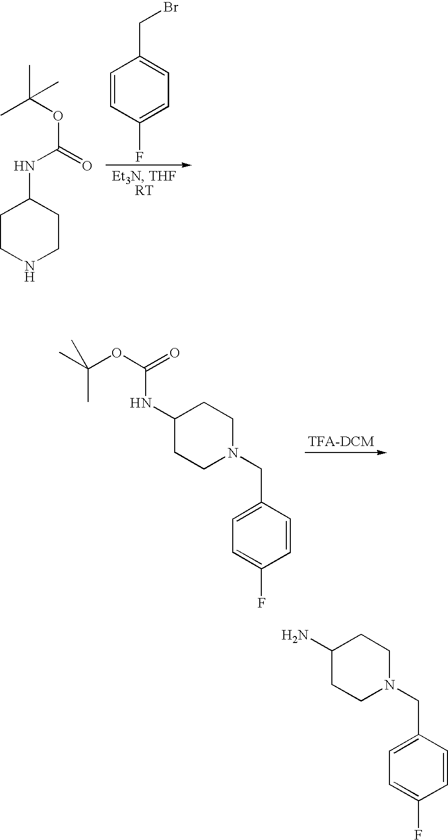

Step-1: [1-(4-fluoro-benzyl)-piperidin-4-yl]carbamic acid tert-butyl ester

To a solution of piperidin-4-yl-carbamic acid tert-butyl ester (1 g, 4.99 mmol) in THF was added triethylamine (1.4 ml, 9.98 mmol) and 4-fluorobenzyl bromide (0.81 ml, 6.49 mmol) at 0° C. The reaction mixture was stirred at room temperature for 3 hour. The mixture was then concentrated, diluted with water and extracted with ethyl acetate. The organic layer was washed with water and dried over sodium sulfate. The organic layer was concentrated to afford 1.4 g (91%) of [1-(4-fluoro-benzyl)-piperidin-4-yl]-carbamic acid tert-butyl ester. LC/MS [M+H]⁺: 309.3

Step-2: 1-(4-Fluoro-benzyl)-piperidin-4-ylamine

To a solution of 1-(4-fluoro-benzyl)-piperidin-4-yl]-carbamic acid tert-butyl ester (1.35 g, 4.3 mmol)) in DCM was added trifluoroacetic acid (0.3 ml, 3.9 mmol). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 900 mg (100%) of 1-(4-fluoro-benzyl)-piperidin-4-ylamine.LC/MS [M+H]⁺:209.2

6-chloro-3,4-dihydro-2H-benzo[1,4]oxazine-2-carboxylic acid [1-(4-fluoro-benzyl)-piperidin-4-yl]-amide was prepared from 6-chloro-2,3-dihydro-benzo[1,4]oxazine -4-carboxylic acid tert-butyl ester-2-carboxylic acid using a process similar to that described in Example 8. LC/MS [M+H]⁺: 404.1. ¹H-NMR (400 MHz, DMSO-d₆) δ (ppm): 7.80-6.17 (8-ArH), 4.44 (m, 1H), 3.50 (m, 1H), 3.44 (m, 1H), 3.40 (m, 1H), 3.37 (m, 2H), 3.28 (m, 1H), 2.67 (m, 1H), 1.98 (m, 2H), 1.66 (m, 2H), 1.51 (m, 2H), 1.23 (m, 1H). HPLC purity: 93.4%

### Example 23

### 1-(6-Chloro-3,4-dihydro-2H-benzo[1,4]oxazine-2-carbonyl)-4-(4-fluoro-benzyl)-piperidine-4-carbonitrile

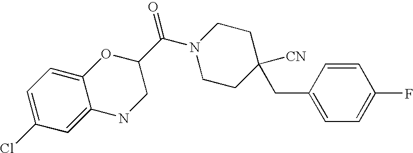

Preparation of 4-(4-Fluoro-benzyl)-piperidine-4-carbonitrile:

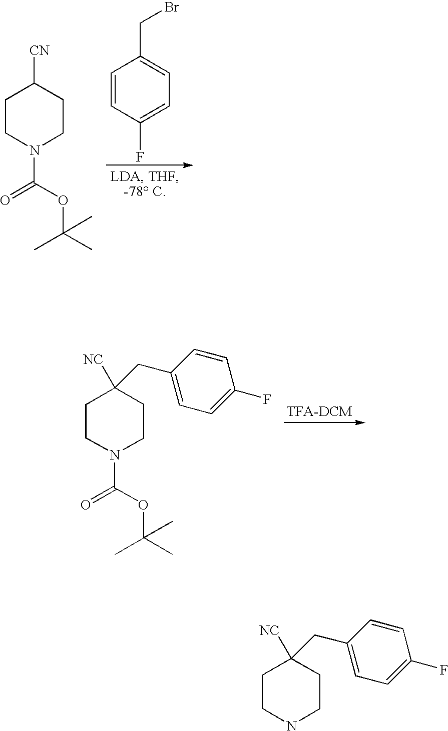

Step-1: 4-Cyano-4-(4-fluoro-benzyl)-piperidine-1-carboxylic acid tent butyl ester

1.6 (M) solution n-butyl lithium in hexane (15.8 ml, 26.6 mmol) was added to an ice-chilled solution of diisopropylamine (3.2 ml, 22.8 mmol) in THF. The resulting mixture was stirred at room temperature for 1 hour. The solution was then cooled to −78° C. and a solution of 4-cyano-1-boc piperidine (4.0 g, 19 mmol) in THF was added dropwise. The mixture was warmed to −40° C. over a period of 1 hour, then cooled to −78° C. A solution of 4-fluorobenzyl bromide in THF was added dropwise and the resuling mixture was stirred at −78° C. for 4 hours then quenched at 0° C. by the addition of saturated aqueous solution of ammonium chloride. The product was extracted with ethyl acetate. The organic layer was washed with brine solution and dried over sodium sulfate. The organic layer was concentrated and the crude mass was crystallized from hexane to afford 3 g (49.6%) of 4-cyano-4-(4-fluoro-benzyl)-piperidine-1-carboxylic acid tert butyl ester. LC/MS [M+H]⁺: 319.4

Step-2: 4-(4-Fluoro-benzyl)-piperidine-4-carbonitrile

Trifluoroacetic acid (5.7 ml, 75.2 mmol) was added to a solution of 4-cyano-4-(4-fluoro-benzyl)-piperidine-1-carboxylic acid tert butyl ester (3 g, 9.4 mmol) in DCM and the resuoing mixture was stirred for about 5 h. The reaction mass was concentrated and washed with dry ether. The TFA salt was diluted with a minimum volume of water and neutralized with aq. NaOH solution at 5-10° C. Extraction of the neutralized solution with ethyl acetate followed by concentration of the organics afforded 2.1 mg (97.6%) of 4-(4-Fluoro-benzyl)-piperidine-4-carbonitrile. LC/MS [M+H]⁺: 219.4

1-(6-Chloro-3,4-dihydro-2H-benzo[1,4]oxazine-2-carbonyl)-4-(4-fluoro-benzyl)-piperidine-4-carbonitrile was prepared from 6-Chloro-2,3-dihydro-benzo[1,4]oxazine-2,4-dicarboxylic acid 4-tert-butyl ester using a process similar to that described in Example 8. LC/MS [M+H]⁺: 414.3. ¹H-NMR (400 MHz, DMSO-d₆) δ (ppm): 7.34 (d, 2H), 7.19 (t, 2H), 6.68 (m, 1H), 6.58 (s, 1H), 6.47 (d, 1H), 6.18 (s, 1H), 4.92 (br s, 1H0, 4.39 (m, 1H), 4.09 (t, 1H), 3.37 (m, 1H), 3.28 (m, 1H), 3.20 (m, 1H), 2.95 (m, 2H), 2.69 (m, 1H), 1.81 (m, 3H), 1.56 (m, 1H), HPLC purity: 99.7%

### Example 24

### [1-(6-Chloro-3,4-dihydro-2H-benzo[1,4]oxazin-2-ylmethyl)-4-(4-fluoro-benzyl)-piperidin-4-yl]-methylamine

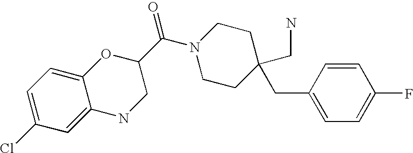

[4-Cyano-4-(4-fluoro-benzyl)-piperidin-1-yl]-(6-chloro-3,4-dihydro-2H-benzo[1,4]oxazin-2-yl)-methanone 100 mg (0.24mmol) in THF was refluxed with BH₃-DMS (0.27 ml, 2.4 mmol) for 20 hours. The reaction mixture was refluxed with 5 ml methanol for one hour then concentrated. Purification of the residue using column chromatography over si-gel afforded 23 mg (24%) of [4-aminomethyl-4-(4-fluoro-benzyl)-piperidin-1-yl]-(6-chloro-3,4-dihydro-2H-benzo[1,4]oxazin-2-yl)-methanone. LC/MS [M+H]⁺: 404.4. ¹H-NMR (400 MHz, DMSO-d₆) δ (ppm): 7.19 (t, 2H), 7.10 (t, 2H), 6.62 (d, 1H), 6.55 (d, 1H), 6.41 (dd, 1H), 6.06 (s, 1H), 5.11 (br s, 2H), 4.08 (m, 1H), 2.96 (m, 1H), 2.66 (s, 2H), 2.50 (m, 1H), 2.45 (m, 2H), 2.32 (m, 4H), 1.34 (m, 6H). HPLC purity: 97.9%

### Example 25

### 2-(6-Chloro-3,4-dihydro-2H-benzo[1,4]oxazin-2-yl)-1-[(R)-4-(4-fluoro-benzyl)-2-methyl-piperazin-1-yl]-ethanone

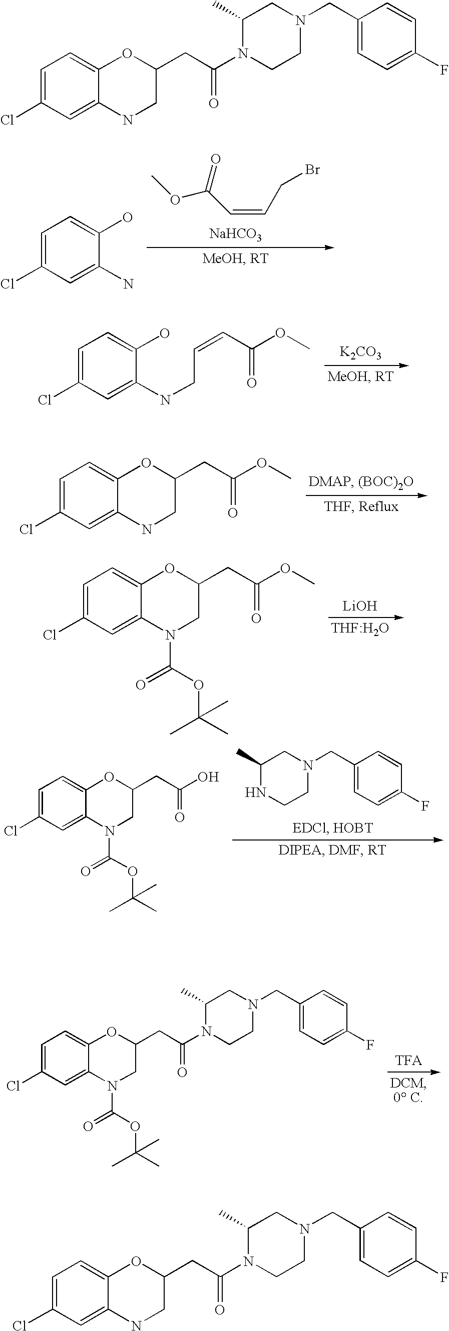

Step-1: (Z)-4-(5-Chloro-2-hydroxy-phenylamino)-but-2-enoic acid methyl ester

NaHCO₃ (1.4 g, 16.7 mmol) and methyl-4-bromocrotonate (1.64 ml, 13.9 mmol) were added to 4-chloro-2-nitrophenol (2 g, 13.9 mmol) in methanol and the resulting mixture was stirred overnight at room temperature. The mixture was then concentrated, diluted with water and extracted with ethyl acetate. The organic layer was dried over sodium sulfate and concentrated under reduced pressure. The crude mass was purified using column chromatography over Si-gel (70% DCM-hexane) to afford 2 g (60%) of (Z)-4-(5-chloro-2-hydroxy-phenylamino)-but-2-enoic acid methyl ester. ¹H-NMR (400 MHz, CDCl₃) δ (ppm): 7.04-6.99 (dt, 1H), 6.64-6.47 (4H), 6.02 (br d, J=16 Hz, 1H), 5.1 (mbr s, 1H), 3.95 (t, 2H), 3.74 (s, 3H)

Step-2: (6-Chloro-3,4-dihydro-2H-benzo[1,4]oxazin-2-yl)-acetic acid methyl ester

A catalytic amount of K₂CO₃ (136 mg, 0.9 mmol) was added to (Z)-4-(5-Chloro-2-hydroxy-phenylamino)-but-2-enoic acid methyl ester (2 g, 8.3 mmol) in methanol and the mixture was stirred at room temperature for 2 hours. The solution was then concentrated, diluted with water and extracted with ethyl acetate. The organic layer was dried over sodium sulfate and concentrated under reduced pressure to afford 1.5 g (75%) of (6-chloro-3,4-dihydro-2H-benzo[1,4]oxazin-2-yl)-acetic acid methyl ester. ¹H-NMR (400 MHz, CDCl₃) δ (ppm): 6.72-6.56 (m, 3H), 4.53 (m, 1H), 3.81 (s, 1H), 3.72 (s, 3H), 3.46 (d, 1H), 3.17 (m, 1H), 2.79-2.58 (m, 2H).

Step-3: 6-Chloro-2-methoxycarbonylmethyl-2,3-dihydro-benzo[1,4]oxazine-4-carboxylic acid tert-butyl ester

DMAP (1.2 g, 9.93 mmol) and (Boc)₂O (2.75 ml, 13.2 mmol) were added to (6-chloro-3,4-dihydro-2H-benzo[1,4]oxazin-2-yl)-acetic acid methyl ester (1.6 g, 6.62mmol) in THF and the resulting mixture was stirred overnight under reflux. The mixture was then concentrated, diluted with water and extracted with ethyl acetate. The organic layer was dried over sodium sulfate and concentrated under reduced pressure. The crude mass was purified using column chromatography over Si-gel (5%-ethyl acetate-hexane) to afford 1.7 g (75%) of 6-chloro-2 methoxycarbonylmethyl-2,3-dihydro-benzo[1,4]oxazine-4-carboxylic acid tert-butyl ester. LC/MS [M+H]⁺: 342. ¹H-NMR (400 MHz, CDCl₃) δ (ppm):7.8 (s, 1H), 6.93-6.76 (m, 2H), 4.59 (m, 1H), 4.1-4.07 (dd, 1H), 3.74 (s, 3H), 3.46 (m, 1H), 2.74-2.61 (m, 2H) 1.56 (s, 9H).

Step-4: 2-Carboxymethyl-6-chloro-2,3-dihydro-benzo[1,4]oxazine-4-carboxylic acid tert-butyl ester

To a solution of 6-chloro-2-methoxycarbonylmethyl-2,3-dihydro-benzo[1,4]oxazine-4-carboxylic acid tert-butyl ester (1.7 g, 4.9 mmol) in THF: H₂O (2:1) was added lithium hydroxide (418 mg, 9.95 mmol). The reaction mixture was stirred for 5-6 h then concentrated. The crude product was neutralized with 50% aqueous acetic acid and extracted with ethyl acetate. Concentration of the ethyl acetate layer afforded 1.35 g (83%) of 2-carboxymethyl-6-chloro-2,3-dihydro-benzo[1,4]oxazine-4-carboxylic acid tert-butyl ester. ¹H-NMR (400 MHz, CDCl₃) δ (ppm):7.8 (s, 1H), 7.06-6.77 (m, 3H), 4.6 (m, 1H), 4.13 (d, 1H), 3.48 (m, 1H), 2.79-2.63 (m, 2H), 1.53 (s, 9H).

Step-5: 6-Chloro-2-{2-[(R)-4-(4-fluoro-benzyl)-2-methyl-piperazin-1-yl]-2-oxo-ethyl}-2,3-dihydro-benzo[1,4]oxazine-4-carboxylic acid tent-butyl ester

(R)-1-(4-fluoro-benzyl)-3-methyl-piperazine (65 mg, 0.31 mmol), EDCI (89 mg, 0.47 mmol), HOBt (50 mg, 0.37 mmol) and DIPEA (0.16 ml, 0.93 mmol) were added to 2-carboxymethyl-6-chloro-2,3-dihydro-benzo[1,4]oxazine-4-carboxylic acid tert-butyl ester (100 mg, 0.31 mmol) in DMF at 5-10° C. The resulting mixture was stirred at room temperature for 14-16 hours then diluted with water and extracted with ethyl acetate. The ethyl acetate layer was concentrated in vacuo and subjected to column chromatography (12% ethyl acetate-hexane) to afford 98 mg (61%) of 6-chloro-2-{2-[(R)-4-(4-fluoro-benzyl)-2-methyl-piperazin-1-yl]-2-oxo-ethyl}-2,3-dihydro-benzo[1,4]oxazine-4-carboxylic acid tert-butyl ester. LC/MS [M+H]: 518.1

Step-6: 2-(6-Chloro-3,4-dihydro-2H-benzo[1,4]oxazin-2-yl)-1-[(R)-4-(4-fluoro-benzyl)-2methyl-piperazin-1-yl]-ethanone

Trifluoroacetic acid (0.1 ml, 1.4 mmol) ws added to a solution of 6-chloro-2-{2-[(R)-4-(4-fluoro-benzyl)-2-methyl-piperazin-1-yl]-2-oxo-ethyl}-2,3-dihydro-benzo[1,4]oxazine-4-carboxylic acid tert-butyl ester (90 mg, 0.17 mmol) in DCM and the resuling mixture was stirred for about 5 h. The reaction mass was then concentrated and washed with dry ether. The TFA salt was diluted with a minimum volume of water and neutralized with aq. NaOH solution at 5-10° C. Extraction of the neutralized solution followed by concentration afforded 30 mg (42%) of 2-(6-chloro-3,4-dihydro-2H-benzo[1,4]oxazin-2-yl)-1-[(R)-4-(4-fluoro-benzyl)-2-methyl-piperazin-1-yl]-ethanone. LC/MS [M+H]⁺: 418.3. ¹H-NMR (400 MHz, DMSO-d₆) δ (ppm): 7.29-6.6 (m, 7H), 4.74 (br s, 1H), 4.59 (br s, 1H), 4.38 (t, 1H), 4.00 (br s, 1H), 3.81 (brs, 1H), 3.55-2.60 (m, 8H), 2.16-1.96 (m, 2H), 1.26 (br s, 3H). HPLC purity: 95.1%

### Example 26

### 2-(6-Chloro-3,4-dihydro-2H-benzo[1,4]oxazin-2-yl)-1-1-[7-(4-fluoro-benzyl)-[1,4]oxazepan-4-yl]-ethanone

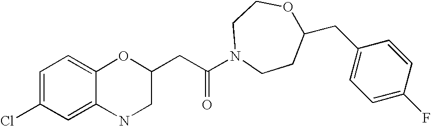

7-(4-Fluoro-benzyl)-[1,4]oxazepane was prepared as described in Example 17.

2-(6-Chloro-3,4-dihydro-2H-benzo[1,4]oxazin-2-yl)-1-[7-(4-fluoro-benzyl)-[1,4]oxazepan-4-yl]-ethanone was prepared by a process similar to that described in Example 25. LC/MS 8 M+H]⁺: 419.2. ¹H-NMR (400 MHz, DMSO-d₆) δ (ppm): 7.25-6.39 (m, 7H), 6.1 (s, 1H), 4.36 (br s, 1H), 3.86-3.39 (m, 5H), 3.00 (m, 2H), 2.77-2.55 (m, 5H), 1.84 (m, 2H), 1.51 (m, 2H). HPLC purity: 90.7%

### Example 27

### 1-(6-Chloro-7-methoxy-3,4-dihydro-2H-benzo[1,4]oxazine-2-carbonyl)-4-(4-fluoro-benzyl)-piperidine-4-carbonitrile

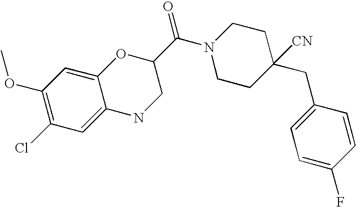

4-(4-Fluoro-benzyl)-piperidine-4-carbonitrile was prepared as described above in Example 23.

Step 1: 2-chloro-5-fluoro-4-nitroanisole

Potassium nitrate (3.15 g, 0.031 mol) was added portion wise at 0° C. to 2-Chloro-5-fluoroanisole (5 g, 0.031 mol) dissolved in conc. H₂SO₄. The reaction mixture was stirred for 1 h. then poured into ice. The resulting precipitate was extracted with ethyl acetate and the organic layer was washed with water, saturated bicarbonate solution and brine. The organics were then dried over Na₂SO₄ and concentrated under reduce pressure to afford 5.5 g (86%) of 2-chloro-5-fluoro-4-nitroanisole. GCMS [m/z]: 205. ¹H-NMR (400 MHz, DMSO-d₆) δ (ppm): 8.30 (d, J=7.9 Hz, 1H), 7.48 (d, J=13.4 Hz, 1H), 4.01 (s, 3H).

Step 2: 4-Chloro-5-methoxy-2-nitrophenol

2-chloro-5-fluoro-4-nitroanisole (2 g, 9.73 mmol) was refluxed overnight with 6 (N) NaOH solution. The pH of the solution was adjusted to 1 with 6(N) HCl. The product was extracted with ethyl acetate, which was washed with water, saturated bicarbonate solution and brine. The organic layer was dried over Na₂SO₄ and concentrated under reduce pressure to afford 1.5 g (75.5%) of 4-chloro-5-methoxy-2-nitrophenol. GCMS [m/z]: 203.5. ¹H-NMR (400 MHz, DMSO-d₆) δ (ppm): 10.9 (s, 1H), 8.14 (s, 1H), 6.59 (s, 1H), 3.97 (s, 3H).

Step 3: Diethyl 4-chloro-5-methoxy-2-nitro-phenyloxymalonate

4-Chloro-5-methoxy-2-nitrophenol (6.3 g, 0.031 mol) was dissolved in a minimum volume of DMF. Anhydrous K₂CO₃ (8.54 g, 0.062 mol) and diethyl-2-bromomalonate (6.8 ml, 0.034 mol) were added to the solution and the resulting mixture was stirred at room temperature overnight. The reaction mixture was diluted with ice-water and extracted with ethyl acetate. The organic layer was washed successively with water and brine and then dried over Na₂SO₄. The organic layer was concentrated under reduced pressure and purification of the residue by flash chromatography (silica-gel, 2% EtOAc-hexane) afforded 4.39 g (40%) of diethyl 4-chloro-5-methoxy-2-nitro-phenyloxymalonate.LC/MS [M+H]⁺: 362.1. ¹H-NMR (400 MHz, DMSO-d₆) δ (ppm): 8.11 (s, 1H), 6.83 (s, 1H), 5.19 (s, 1H), 4.33 (m, 4H), 3.94 (s, 3H), 1.32 (m, 6H)

Step 4: Ethyl-6-Chloro-7-methoxy-3-oxo-3,4-dihydro-2H-benzo[1,4]oxazine-2-carboxylate

4-chloro-5-methoxy-2-nitro-phenyloxymalonate (725 mg, 2 mmol) was dissolved in EtOH: H₂O (4:5) and Na₂S₂O₄.H₂O (2.34 g, 12 mmol) was added. The reaction mixture was stirred at 60° C. for 5 h. The reaction mixture was then concentrated under reduced pressure, diluted with water and extracted with ethyl acetate. The organic layer was washed with water and brine, dried over Na₂SO₄ and concentrated under reduce pressure to obtain 200 mg (35%) of ethyl-6-chloro-7-methoxy-3-oxo-3,4-dihydro-2H-benzo[1,4]oxazine-2-carboxylate. LC/MS [M+H]⁺: 286.2. ¹H-NMR (400 MHz, DMSO-d₆) δ (ppm): 7.84 (br s, 1H), 6.99 (s, 1H), 6.73 (s, 1H), 4.26 (m, 2H), 1.28 (t, J=6.8 Hz, 3H).

Step 5: Ethyl-6-Chloro-7-methoxy-3,4-dihydro-2H-benzo[1,4]oxazine-2-carboxylate

BH₃-DMS (0.17 ml, 2.17 mmol) was added at 0° C. to a solution of ethyl-6-chloro-7-methoxy-3-oxo-3,4-dihydro-2H-benzo[1,4]oxazine-2-carboxylate (620 mg, 2.17 mmol) in THF and the resulting mixture was stirred overnight at room temperature. The mixture was then cooled to 0° C., quenched with MeOH and the solution was refluxed for 1 h. The reaction mixture was concentrated under reduced pressure. Purification of the residue by flash chromatography (silica-gel, 8% EtOAc-hexane) afforded 150 mg (25%) of ethyl-6-Chloro-7-methoxy-3,4-dihydro-2H-benzo[1,4]oxazine-2-carboxylate. LC/MS [M+H]⁺: 272.3. ¹H-NMR (400 MHz, DMSO-d₆) δ (ppm): 6.63 (s, 2H), 5.61 s, 1H), 4.95 (br s, 1H), 4.13 (q, J=7 Hz, 2H), 3.71 (s, 3H), 1.8 (t, J=7 Hz, 3H)

Step 6: 6-Chloro-7-methoxy-3,4-dihydro-2H-benzo[1,4]oxazine-2-carboxylic acid

Lithium hydroxide (71 mg, 1.69 mmol) was added to a solution of ethyl-6-chloro-7-methoxy-3,4-dihydro-2H-benzo[1,4]oxazine-2-carboxylate (230 mg, 0.84 mmol) in THF: H₂O (2:1) and the resulting solution was stirred for 5-6 h then concentrated. The crude product was neutralized with 50% aqueous acetic acid and extracted with ethyl acetate. Concentration of the ethyl acetate layer afforded 170 mg (83%) of 6-chloro-7-methoxy-3,4-dihydro-2H-benzo[1,4]oxazine-2-carboxylic acid. LC/MS [M+H]⁺: 244.3. ¹H-NMR (400 MHz, DMSO-d₆) δ (ppm): 6.54 (s, 1H), 6.50 (s, 1H), 5.37 (s, 1H), 4.07(d, J=4.9, 1H), 3.68 (s, 3H), 3.08 (m, 1H).

Step-6: (6-Chloro-7-methoxy-2,3-dihydro-benzo[1,4]dioxin-2-yl)-[{(4-fluorophenyl)methyl}-1-piperidinyl}]-methanone

To a solution of 6-chloro-7-methoxy-3,4-dihydro-2H-benzo[1,4]oxazine-2-carboxylic acid (130 mg, 0.53 mmol) in DMF was added 4-cyano-4-(4-fluoro-benzyl)-piperidine (126.5 mg, 0.0.58 mmol), EDCI (152.8 mg, 0.79 mmol), HOBt (86.34 mg, 0.63 mmol) and DIPEA (0.12 ml, 1.6 mmol) at 5-10° C. and the resulting mixture was stirred at room temperature for 14-16 hours. The mixture was then diluted with water and extracted with ethyl acetate. The organic layer was concentrated under reduced pressure and the residue was purified by flash chromatography (silica-gel, 2% MeOH-DCM) to afford 22 mg (7%) of (6-chloro-7-methoxy-2,3-dihydro-benzo[1,4]dioxin-2-yl)-[{(4-fluorophenyl)methyl}-1-piperidinyl}]-methanone. LC/MS [M+H]⁺:444.3.

¹H-NMR (400 MHz, DMSO-d₆) δ (ppm): 7.34 (br s, 2H), 7.18 (m, 2H), 6.64-6.56 (m, 2H), 5.58 (s, 1H), 4.97 (m, 1H), 4.37 (m, 1H) 4.08 (m, 1H), 3.83 (m, 1H) 3.71 (s, 3H), 3.21 (m, 1H), 2.95 (m, 1H),1.87 (m, 3H), 1.54 (m, 1H). HPLC purity: 99.23%.

### Example 28

### 2-(6-Chloro-3,4-dihydro-2H-benzo[1,4]oxazin-2-yl)-1-[4-(4-fluoro-benzyl)-[1,4]diazepan-1-yl]-ethanone

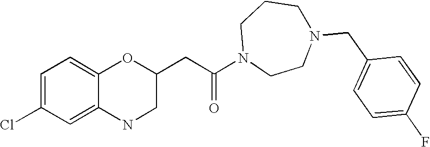

2-(6-chloro-3,4-dihydro-2H-benzo[1,4]oxazin-2-yl)-[4-(4-fluoro-benzyl)-[1,4]diazepan-1-yl]-ethanone was prepared using a procedure similar to that described in Example 25. LC/MS [M+H]⁺: 418.3. ¹H-NMR (400 MHz, DMSO-d₆) δ (ppm): 7.25-6.56 (m, 7H), 4.64 (m, 1H), 3.81-3.48 (m, 8H), 3.19 (m, 1H), 2.79 (m, 1H), 2.62 (m, 4H), 1.84 (m, 2H). HPLC purity: 95.2%

### Example 29

### 2-(6-Chloro-3,4-dihydro-2H-benzo[1,4]oxazin-2-yl)-1-[2-(4-fluoro-benzyl)-(1,4]oxazepan-4yl]-ethanone

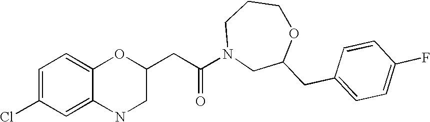

Preparation of 2-(4-fluoro-benzyl)-[1,4]oxazepane

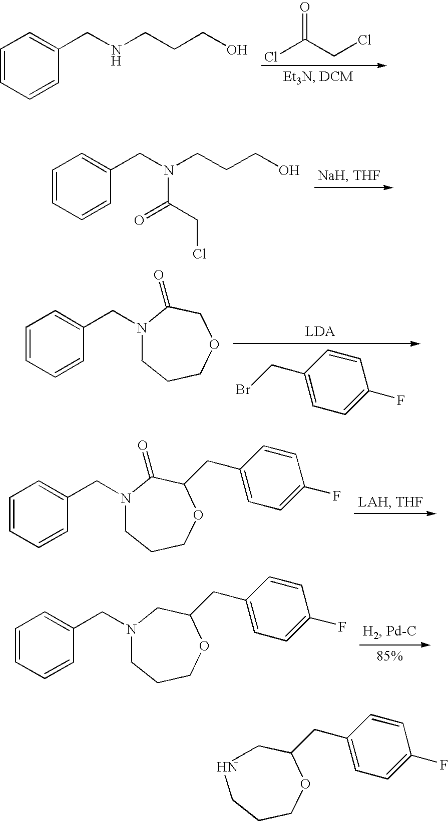

Step-1: N-Benzyl-2-chloro-N-(3-hydroxy-propyl)-acetamide

To a solution of 3-benzylamino-propan-1-ol (28 g, 0.17 mol) solution in DCM was added triethylamine (25.9 ml, 0.19 mol) and chloroacetyl chloride (13.4 ml, 0.17 mol) at 0° C. The mixture was stirred for 2 h at room temperature, then concentrated and extracted with ethyl acetate. Concentration of organic layer afforded 41 g (93%) of N-benzyl-2-chloro-N-(3-hydroxy-propyl)-acetamide. LC/MS [M+H]⁺: 242.3

Step-2: 4-Benzyl-[1,4]oxazepan-3-one

A solution of N-benzyl-2-chloro-N-(3-hydroxy-propyl)-acetamide (30 g, 0.12 mol) in THF was added to a suspension of sodium hydride (6.25 g, 0.13 mol) in dry THF at 0° C. and the resulting mixture was stirred overnight. Excess sodium hydride was quenched with saturated NH₄Cl solution and extracted with ethyl acetate. The organic layer was washed with water and brine solution. The combined organic phase was concentrated under reduced pressure and purified through column chromatography (Si-gel, 1-2% MeOH-DCM) to afford 16 g of 4-benzyl-[1,4]oxazepan-3-one in 68% yield. LC/MS [M+H]⁺:206.2. ¹H-NMR (400 MHz, DMSO-d₆) δ (ppm): 7.35-7.24 (m, 5H), 4.51 (s, 2H), 4.19 (s, 2H), 3.73 (t, J=5.6 Hz, 2H), 3.43 (t, J=4.9 Hz, 2H), 1.69 (m, 2H)

Step-3: 4-Benzyl-2-(4-fluoro-benzyl)-[1,4]oxazepan-3-one

LDA solution in THF (5.9 mmol) was added to a solution of 4-benzyl-[1,4]oxazepan-3-one (1 g, 4.9 mmol) in THF at −78° C. The solution was stirred for 1 h at −78° C. then 4-fluorobenzyl bromide (920 mg, 4.9 mmol) was added. The solution was allowed to warm to room temperature over a period of 4 h. The reaction mixture was then quenched with saturated NH₄Cl solution and extracted with ethyl acetate. The organic layer was washed with water and brine solution. The organic phase was concentrated in vacuo and purified through column chromatography (Si-gel, 5-10% ethyl acetate-hexane) to afford 1.1 g (49%) of 4-benzyl-2-(4-fluoro-benzyl)-[1,4]oxazepan-3-one. LC/MS [M+H]⁺:314.2. ¹H-NMR (400 MHz, DMSO-d₆) δ (ppm): 7.35-7.06 (9 Ar H), 4.59-4.41 (m, 3H), 3.92 (m, 1H), 3.69 (m, 1H), 3.41 (m, 1H), 3.3 (m, 1H), 3.04 (dd, J=14.5, 3.7 Hz, 1H), 2.80 (m, 1H), 1.53 (s, 2H).

Step-4: 4-Benzyl-2-(4-fluoro-benzyl)-[1,4]oxazepane

Lithium aluminum hydride (726 mg, 19.1 mmol) was added portion wise at 0° C. o a solution of 4-benzyl-2-(4-fluoro-benzyl)-[1,4]oxazepan-3-one (3.08 g, 9.6 mmol) in THF. The mixture was refluxed for 2 h and excess LiALH₄ was quenched with aq. NaOH solution at 0° C. The reaction mixture was filtered through celite bed. Concentration of the organic layer afforded 2.5 g (89%) of 4-benzyl-2-(4-fluoro-benzyl)-[1,4]oxazepane. LC/MS [M+H]⁺:300.2. ¹H-NMR (400 MHz, DMSO-d₆) δ (ppm): 7.29-7.01 (9 Ar H), 3.73 (m, 2H), 3.56 (m, 3H), 2.78-2.31 9 m, 5H), 1.79-1.70 (2H).

Step-5: 2-(4-Fluoro-benzyl)-[1,4]oxazepane

4-benzyl-2-(4-fluoro-benzyl)-[1,4]oxazepane (2.5 g) was hydrogenated using 10% Pd-C in ethanol for 5-10 h. The catalyst was removed by filtration through a celite bed. Concentration of the ethanol solution afforded 1.8 g (97%) of 2-(4-fluoro-benzyl)-[1,4]oxazepane. LC/MS [M+H]⁺:210.3. ¹H-NMR (400 MHz, DMSO-d₆) δ (ppm): 7.22-7.05 (4 Ar H), 3.75 (m, 1H), 3.54 (m, 2H), 3.88 (m, 2H), 2.63 (m, 3H), 2.41 9 m, 1H), 1.68 (m, 2H).

2-(6-chloro-3,4-dihydro-2H-benzo[1,4]oxazin-2-yl)-1-[2-(4-fluoro-benzyl)-[1,4]oxazepan-4-yl]-ethanone was prepared using a procedure similar to that described in Example 25. LC/MS [M+H]⁺: 419.3. ¹H-NMR (400 MHz, DMSO-d₆) δ (ppm): 7.28-7.06 (m, 7H), 6.09 (s, 1H), 4.36 (m, 1H), 4.02-3.66 (m, 4H), 3.38 (m, 2H), 3.14-2.60 (m, 7H), 1.80 m, 2H). HPLC purity: 95.47%

### Example 30

### 6-Chloro-2-{2-[2-(4-fluoro-benzyl)-[1,4]oxazepan-4-yl]-ethyl}-3,4-dihydro-2H-benzo[1,4]oxazine

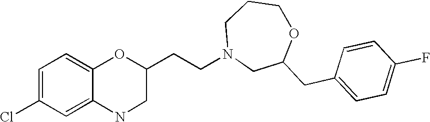

A solution of 2-(6-chloro-3,4-dihydro-2H-benzo[1,4]oxazin-2-yl)-1-[2-(4-fluoro-benzyl)-[1,4]oxazepan-4-yl]-ethanone (100 mg, 0.24 mmol) in THF was refluxed with BH₃-THF (0.13 ml, 1.19 mmol) for 8 hours. The reaction mixture was refluxed with 5 ml methanol for an additional hour, the concentrated. Purification of the residue using column chromatography over si-gel (1.5% MeOH-DCM) afforded 62 mg (64.5%) of 6-chloro-2-{2-[2-(4-fluoro-benzyl)-[1,4]oxazepan-4-yl]-ethyl}-3,4-dihydro-2H-benzo[1,4]oxazine. LC/MS [M+H]⁺: 405.4. ¹H-NMR (400 MHz, DMSO-d₆) δ (ppm): 7.30-6.41 (m, 7H), 6.07 (s, 1H), 3.97 (br s, 1H), 3.97-3.49 (m, 5H), 2.98-2.50 (m, 7H), 2.34 (m, 1H), 1.70 (m, 4H). HPLC purity: 98.1%

### Example 31

### 4-(4-Chloro-benzyl)-1-(6-chloro-3,4-dihydro-2H-benzo[1,4]oxazine-2-carbonyl)-piperidine-4-carbonitrile

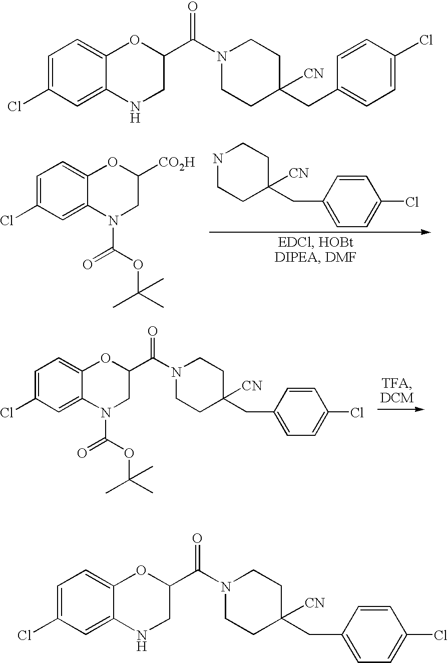

Preparation of 4-(4-Chloro-benzyl)-piperidine-4-carbonitrile

4-(4-chloro-benzyl)-piperidine-4-carbonitrile was prepared using a procedure similar to that described in Example 23. LC/MS [M+H]⁺: 219.2. ¹H-NMR (400MHz, DMSO-d₆) δ (ppm): 7.33-7.15 (4 Ar H), 2.90 (t, J=12.8 Hz, 2H), 2.86 (s, 2H), 2.55 (t, J=12.2 Hz, 2H), 1.65 (d, J=12.9 Hz, 2H), 1.46 (t, J=3.4 Hz, 2H). HPLC purity: 99%

6-chloro-2,3-dihydro-benzo[1,4]oxazine -4-carboxylic acid tert-butyl ester-2-carboxylic acid was prepared using a procedure similar to that described in in Example 8. GCMS [m/z-Boc]: 213. ¹H-NMR (400MHz, DMSO-d₆) δ (ppm): 13.40 (br s, 1H), 7.72-6.95 (3 Ar H), 5.06 (br, s, 1H), 4.40 (dd, J=11.2, 2.6 Hz, 1H), 3.55 (dd, J=10.8, 3.0 Hz, 1H), 1.38 (s, 9H)

Step 1: 6-Chloro-2-[4-(4-chloro-benzyl)-4-cyano-piperidine-1-carbonyl]-2,3-dihydro-benzo[1,4]oxazine-4-carboxylic acid tent-butyl ester

To a solution of 6-chloro-2,3-dihydro-benzo[1,4]oxazine-4-carboxylic acid tert-butyl ester-2-carboxylic acid (200 mg, 0.64 mmol) in DMF was added 4-cyano-4-(4-chloro-benzyl)-piperidine (140 mg, 0.64 mmol), EDCI (160 mg, 0.83 mmol), HOBt (43 mg, 0.32 mmol) and DIPEA (0.32 ml, 1.42 mmol) at 5-10° C. and the resulting mixture was stirred at room temperature for 14-16 hours. The mixture was then diluted with water and extracted with ethyl acetate. The organic layer was concentrated under reduced pressure and the residue was purified by flash chromatography (silica-gel, 2% MeOH-DCM) to afford 230 mg (70%) of 6-Chloro-2-[4-(4-chloro-benzyl)-4-cyano-piperidine-1-carbonyl]-2,3-dihydro-benzo[1,4]oxazine-4-carboxylic acid tert-butyl ester. LC/MS [M+H]⁺: 514.2. ¹N-NMR (400 MHz, DMSO-d₆) δ (ppm): 7.67-6.92 (7 Ar H), 5.44 (s, 1H), 5.37 (s, 1H), 4.33 (d, J=12.9 Hz, 1H), 4.10 (m, 2H), 3.82 (m, 3H), 2.95 (s, 2H), 1.78 (m, 2H), 1.45 (s, 9H), 1.23 (s, 1H).

Step-2: 4-(4-Chloro-benzyl)-1-(6-chloro-3,4dihydro-2H-benzo[1,4]oxazine-2-carbonyl)-piperidine-4-carbonitrile

Trifluoroacetic acid (1.72 ml, 22.3 mmol) was added to a solution of 6-Chloro-2-[4-(4-chloro-benzyl)-4-cyano-piperidine-1-carbonyl]-2,3-dihydro-benzo[1,4]oxazine-4-carboxylic acid tert-butyl ester (230 mg, 0.45 mmol) in DCM at 5-10° C. and the reaction mixture was stirred for 2-4 h at room temperature. The solution was concentrated and basified with 1 (N) NaOH solution. The aqueous solution was extracted with ethyl acetate and the organic layer was washed with water and brine solution. Concentration of the organic layer afforded 140 mg (29%) of 4-(4-Chloro-benzyl)-1-(6-chloro-3,4-dihydro-2H-benzo[1,4]oxazine-2-carbonyl)-piperidine-4-carbonitrile. LC/MS [M+H]⁺: 414.3. ¹H-NMR (400MHz, DMSO-d₆) δ (ppm): 7.34 (m, 2H), 7.19 (t, J=8.7 Hz, 2H), 6.69 (m, 1H), 6.58 (s, 1H), 6.47 (d, J=8.2 Hz, 1H), 6.18 (s, 1H), 4.92 (m, 1H), 4.39 (m, 1H), 4.07 (m, 1H), 3.40-2.67 (m, 6H), 1.88 (m, 3H), 1.55 (m, 1H). HPLC purity: 90.80%

### Example 32

### [4-(4-Chloro-benzyl)-4-hydroxy-3,3-dimethyl-piperidin-1-yl]-(6-chloro-3,4-dihydro-2H-benzo[1,4]oxazin-2-yl)-methanone

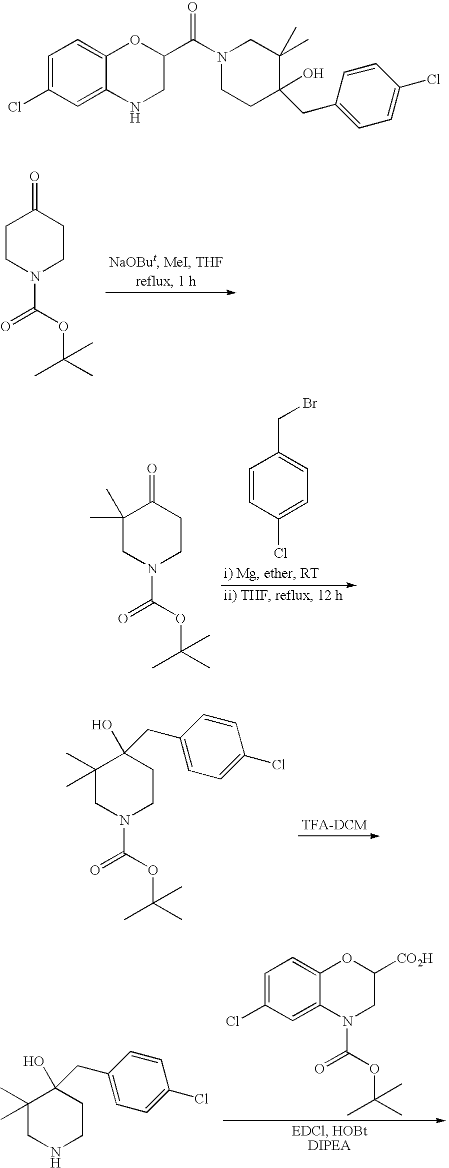

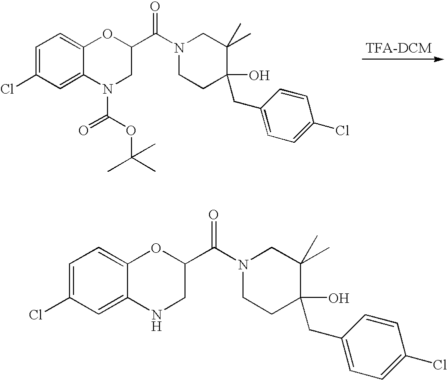

6-Chloro-2,3-dihydro-benzo[1,4]oxazine -2,4-dicarboxylic acid 4-tert-butyl ester was prepared as described in Example 8.

Step-1: 3,3-Dimethyl-4-oxo-piperidine-1-carboxylic acid tert-butyl ester

Methyl iodide (11.8 ml, 0.19 mol) was added at 0° C. to a solution of 4-oxo-piperidine-1-carboxylic acid tert-butyl ester (18 g, 0.09 mol). Sodium tert butoxide (20.9 g, 0.22 mol) was then added at 0° C. and the resulting mixture was heated to reflux for 1 hour. The reaction mixture was concentrated under reduced pressure, diluted with water and the product was extracted with ethyl acetate. The organic layer was washed with brine, dried over sodium sulfate and concentrated Purification of the residue by column chromatography (2% ethyl acetate-hexane) afforded 7 g (37%) of 3,3-dimethyl-4-oxo-piperidine-1-carboxylic acid tert-butyl ester. ¹H-NMR (400 MHz, CDCl₃) δ (ppm): 3.7 (m, 2H), 3.41 (m, 2H), 2.47 (t, 2H), 1.48 (s, 9H), 1.09 (s, 6H).

Step-2: 4-(4-Chloro-benzyl)-4-hydroxy-3,3-dimethyl-piperidine-1-carboxylic acid tert-butyl ester

Magnesium turnings (500 mg, 20.57 mmol) were added to a solution of 4-chlorobenzyl bromide (3.52 g, 17.2 mmol) in ether and the mixture was stirred at room temperature for 1 hour. A solution of 3,3-dimethyl-4-oxo-piperidine-1-carboxylic acid tert-butyl ester (3.0 g, 13.2 mmol) in THF was then added dropwise at room temperature and the mixture was heated to reflux overnight. The mixture was then diluted with aqueous saturated NH4Cl solution and extracted with ethyl acetate. The organic layer was washed with water, dried over sodium sulfate, concentrated and purified through Si-gel (5% ether-hexane) to afford 1.8 g (38.5%) of 4-(4-chloro-benzyl)-4-hydroxy-3,3-dimethyl-piperidine-1-carboxylic acid tert-butyl ester. LC/MS [M+H]⁺: 354.3

Step-3: 4-(4-Chloro-benzyl)-3,3-dimethyl-piperidin-4-ol

Trifluoroacetic acid (3.8 ml, 50 mmol) was added to a solution of 4-(4-chloro-benzyl)-4-hydroxy-3,3-dimethyl-piperidine-1-carboxylic acid tert-butyl ester (1.8 g, 5.08 mmol)) in DCM and the resulting mixture was stirred for about 5 h. The reaction mass was concentrated and washed with dry ether. The TFA salt was diluted with a minimum volume of water and neutralized with aq. NaOH solution at 5-10° C. Extraction of the neutralized solution followed by concentration afforded 1 g (77.6%) of 4-(4-chloro-benzyl)-3,3-dimethyl-piperidin-4-ol. LC/MS [M+H]⁺: 254.2

Step-4: 6-Chloro-2-[4-(4-chloro-benzyl)-4-hydroxy-3,3-dimethyl-piperidine-1-carbonyl]-2,3-dihydro-benzo[1,4]oxazine-4-carboxylic acid tert-butyl ester

To a solution of 6-chloro-2,3-dihydro-benzo[1,4]oxazine -4-carboxylic acid tert-butyl ester-2-carboxylic acid (150 mg, 0.48 mmol) in DMF was added 4-(4-chloro-benzyl)-3,3-dimethyl-piperidin-4-ol (121 mg, 0.48 mmol), EDCI (275 mg, 1.43 mmol), HOBt (33 mg, 0.24 mmol) and DIPEA (0.28 ml, 1.67 mmol) at 5-10° C. and the mixture was stirred at room temperature for 14-16 hours. The mixture was then diluted with water and extracted with ethyl acetate. The organic layer was concentrated under reduced pressure and purification of the residue by flash chromatography (silica-gel, 2% MeOH-DCM) afforded 110 mg (42%) of 6-chloro-2-[4-(4-chloro-benzyl)-4-hydroxy-3,3-dimethyl-piperidine-1-carbonyl]-2,3-dihydro-benzo[1,4]oxazine-4-carboxylic acid tert-butyl ester. LC/MS [M+H]⁺: 549.4. ¹H-NMR (400MHz, DMSO-d₆) δ (ppm): 7.85 (m, 1H), 7.31 (m, 2H), 7.11 (m, 2H), 6.93 (m, 1H), 6.83 (m, 1H), 4.85 (m, 1H), 4.35 (m, 1H), 3.71 (m, 2H), 2.90 (m, 1H) 2.64 (m, 1H), 1.77 (m, 1H), 1.54 (s, 9H), 1.26 (m, 1H), 1.06 (m, 6H), 1.03 (m, 2H).

Step-5: [4-(4-Chloro-benzyl)-4-hydroxy-3,3-dimethyl-piperidin-1-yl]-(6-chloro-3,4-dihydro-2H-benzo[1,4]oxazin-2-yl)-methanone

Trifluoroacetic acid (1.4 ml, 2.0 mmol) was added at 5-10° C. to a solution of 6-chloro-2-[4-(4-chloro-benzyl)-4-hydroxy-3,3-dimethyl-piperidine-1-carbonyl]-2,3-dihydro-benzo[1,4]oxazine-4-carboxylic acid tert-butyl ester (100 mg, 0.2 mmol) in DCM. The resulting mixture was stirred for 2-4 h at room temperature, then concentrated and basified with 1 (N) NaOH solution. The aqueous solution was extracted with ethyl acetate and the organic layer was washed with water and brine solution. Concentration of the organic layer afforded 65 mg (72.5%) of [4-(4-chloro-benzyl)-4-hydroxy-3,3-dimethyl-piperidin-1-yl]-(6-chloro-3,4-dihydro-2H-benzo[1,4]oxazin-2-yl)-methanone. LC/MS [M+H]⁺: 449.3. ¹H-NMR (400 MHz, DMSO-d₆) δ (ppm): 7.29 (m, 4H), 6.67 (m, 1H), 6.61 (s, 1H), 6.47 (m, 1H), 5.92 (br s, 1H), 3.99 (s, 1H), 3.77 (m, 1H), 3.35 (m, 4H), 2.82 (d, 1H), 2.68 (m, 1H), 1.61 (m, 1H), 1.18 (d, 1H), 0.86 (m, 1H), 0.82 (m, 6H). HPLC purity: 98.2%

### Example 33

### 4-(4-Chloro-benzyl)-1-(6-chloro-3,4-dihydro-2H-benzo[1,4]oxazin-2-ylmethyl)-3,3-dimethyl-piperidin-4-ol

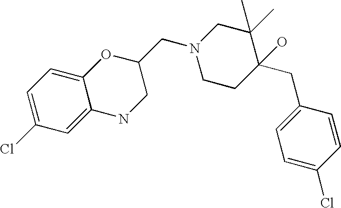

To a solution of [4-(4-chloro-benzyl)-4-hydroxy-3,3-dimethyl-piperidin-1-yl]-(6-chloro-3,4-dihydro-2H-benzo[1,4]oxazin-2-yl)-methanone (prepared as described in Example 32) (30 mg, 0.07 mmol) in THF was added BH₃-DMS (0.03 ml, 0.33 mmol) at 0° C. The reaction mixture was stirred overnight at room temperature. The mixture was cooled to 0° C., quenched with MeOH then heated to reflux for 1 h. Concentration under reduced pressure followed by purification by flash chromatography (silica-gel, 0-0.5% MeOH-DCM) afforded 20 mg (67%) of 4-(4-chloro-benzyl)-1-(6-chloro-3,4-dihydro-2H-benzo[1,4]oxazin-2-ylmethyl)-3,3-dimethyl-piperidin-4-ol. LC/MS [M+H]⁺: 435.2. ¹H-NMR (400 MHz, DMSO-d₆) δ (ppm): 7.26 (m, 4H), 6.61 (d, J=8.28, 1H), 6.55 (d, J=1.76 , 1H) 6.42 (dd, 1H), 6.06 (s, 1H), 4.06 (d, 1H), 3.87 (s, 1H), 3.00 (q, 1H), 2.72 (d, 1H), 2.63 (m, 1H), 2.42 (m, 2H), 2.13 (m, 1H), 1.53 (m, 1H), 1.05 (s, 3H), 0.88 (s, 3H). HPLC purity: 98.8%

### Example 34

### [4-(4-Chloro-benzyl)-4-hydroxy-3,3-dimethyl-piperidin-1-yl]-(6-chloro-2,3-dihydro-benzo[1,4]dioxin-2-yl)-methanone

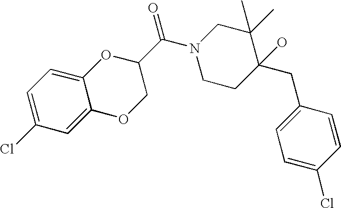

4-(4-Chloro-benzyl)-3,3-dimethyl-piperidin-4-ol was prepared as described in Example 32.

[4-(4-Chloro-benzyl)-4-hydroxy-3,3-dimethyl-piperidin-1-yl]-(6-chloro-2,3-dihydro-benzo[1,4]dioxin-2-yl)-methanone was prepared using 4-(4-Chloro-benzyl)-3,3-dimethyl-piperidin-4-ol coupling with 6-Chloro-2,3-dihydro-benzo[1,4]dioxine-2-carboxylic acid by a procedure similar to that described in Example 1. LC/MS [M+H]⁺: 450.2. ¹H-NMR (400MHz, DMSO-d₆) δ (ppm):7.31-6.87 (m, 7H), 5.31 (m, 1H), 4.36 -3.37 (m, 5H), 3.06-2.08 (m, 5H), 1.23 (m, 1H), 0.94 (s, 6H). HPLC purity: 93.1%

### Example 35

### 4-(4-Chloro-benzyl)-1-(6-chloro-2,3-dihydro-benzo[1,4]dioxin-2-ylmethyl)-3,3-dimethyl-piperidin-4-ol

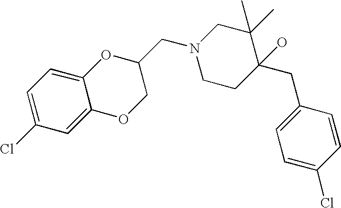

To a solution of [4-(4-chloro-benzyl)-4-hydroxy-3,3-dimethyl-piperidin-1-yl]-(6-chloro-2,3-dihydro-benzo[1,4]dioxin-2-yl)-methanone (40 mg, 0.09 mmol) in THF was added BH₃-DMS (0.04 ml, 0.44 mmol) at 0° C. The reaction mixture was stirred overnight at room temperature. The reaction mixture was then cooled to 0° C., quenched with MeOH and the solution was heated to reflux for 1 h. The reaction mixture was concentrated under reduced pressure. Purification of the residue by flash chromatography (silica-gel, 2.5% EtOAc-hexane) afforded 20 mg (51%) of 4-(4-chloro-benzyl)-1-(6-chloro-2,3-dihydro-benzo[1,4]dioxin-2-ylmethyl)-3,3-dimethyl-piperidin-4-ol. LC/MS [M+H]⁺:436.3 ¹H-NMR (400 MHz, CDCl₃) δ (ppm): 7.27-6.74 (m, 7H), 4.33-3.93 (m, 3H), 2.89-2.16 (m, 10H), 1.15 (s, 3H), 0.99 (s, 3H). HPLC purity: 96.2%

### Example 36

### 1-(6-Chloro-7-methoxy-4-methyl-3,4-dihydro-2H-benzo[1,4]oxazine-2-carbonyl)-4-(4-fluoro-benzyl)-piperidine-4-carbonitrile

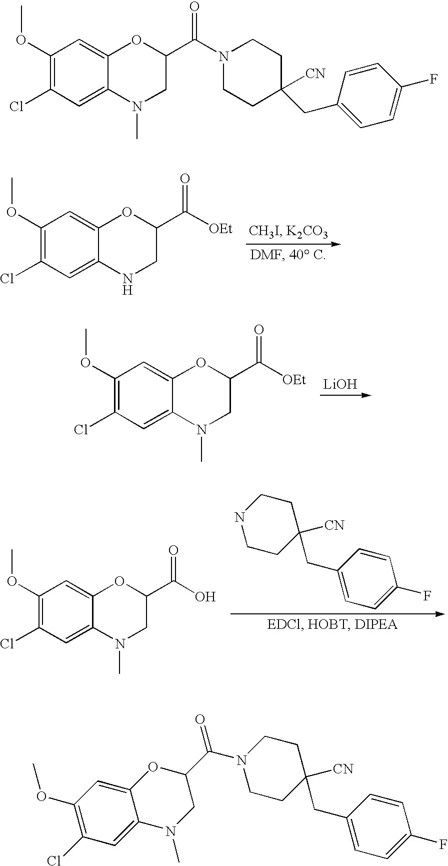

Step-1: 6-Chloro-7-methoxy-4-methyl-3,4-dihydro-2H-benzo[1,4]oxazine-2-carboxylic acid ethyl ester

Anhydrous K₂CO₃ (458 mg, 3.3 mmol) and methyl iodide (0.2 ml, 3.3 mmol) were added to a solution of 6-Chloro-7-methoxy-3,4-dihydro-2H-benzo[1,4]oxazine-2-carboxylic acid ethyl ester (prepared as described in Example 27) (300 mg, 1.1 mmol) in DMF and the resulting mixture was stirred at 40° C. for 3 hours. The mixture was diluted with crushed ice and extracted with ethyl acetate. The organic layer was concentrated and purified through column chromatography over Si-gel (7% ethyl acetate-hexane) to afford 167 mg (53%) of 6-Chloro-7-methoxy-4-methyl-3,4-dihydro-2H-benzo[1,4]oxazine-2-carboxylic acid ethyl ester. LC/MS [M+H]⁺: 286.2

Step-2: 6-Chloro-7-methoxy-4-methyl-3,4-dihydro-2H-benzo[1,4]oxazine-2-carboxylic acid

Lithium hydroxide (48 mg, 1.15 mmol) was added to a solution of 6-Chloro-7-methoxy-4-methyl-3,4-dihydro-2H-benzo[1,4]oxazine-2-carboxylic acid ethyl ester (165 mg, 0.58 mmol) in THF:H₂O (2:1) and the resuling mixture was stirred for 5-6 h, then concentrated the crude product was neutralized with 50% aqueous acetic acid and extracted with ethyl acetate. Concentration of ethyl acetate layer afforded 110 mg (74%) of 6-chloro-7-methoxy-4-methyl-3,4-dihydro-2H-benzo[1,4]oxazine-2-carboxylic acid. LC/MS [M+H]⁺: 258.4

Step-3: 1-(6-Chloro-7-methoxy-4-methyl-3,4-dihydro-2H-benzo[1,4]oxazine-2-carbonyl)-4-(4-fluoro-benzyl)-piperidine-4-carbonitrile

To a solution of 6-chloro-7-methoxy-4-methyl-3,4-dihydro-2H-benzo[1,4]oxazine-2-carboxylic acid (110 mg, 0.43 mmol) in DMF was added 4-(4-Chloro-benzyl)-piperidine-4-carbonitrile (93 mg, 0.0.43 mmol), EDCI (106 mg, 0.55 mmol), HOBt (29 mg, 0.22 mmol) and DIPEA (0.15 ml, 0.85 mmol) at 5-10° C. and the mixture was stirred at room temperature for 14-16 hours. The reaction mixture was diluted with water and extracted with ethyl acetate. The organic layer was concentrated under reduced pressure and the residue was purified by flash chromatography (silica-gel, 2% MeOH-DCM) to afford 145 mg (75%) of 1-(6-Chloro-7-methoxy-4-methyl-3,4-dihydro-2H-benzo[1,4]oxazine-2-carbonyl)-4-(4-fluoro-benzyl)-piperidine-4-carbonitrile. LC/MS [M+H]⁺: 458.3. ¹H-NMR (400 MHz, DMSO-d₆) δ (ppm): 7.35-6.59 (m, 7H), 5.2 (br s, 1H), 4.38 (m, 1H), 4.08 (m, 1H), 3.72 (s, 3H), 3.27-3.12 (m, 3H), 2.96 (s, 2H), 2.77 (s, 3H), 2.68 (m, 1H), 1.86-1.23 (m, 4H). HPLC purity: 91.4%

### Example 37

### 1-(6-Chloro-7-hydroxy-3,4-dihydro-2H-benzo[1,4]oxazine-2-carbonyl)-4-(4-fluoro-benzyl)-piperidine-4-carbonitrile

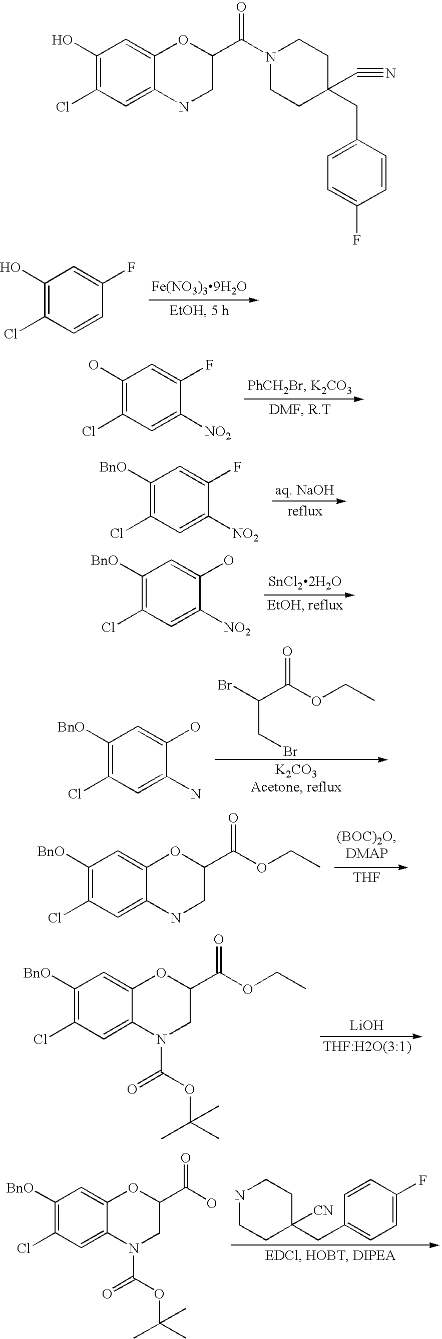

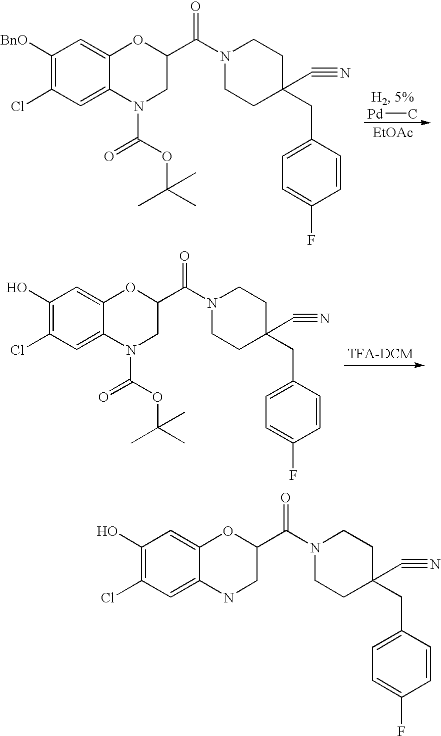

Step-1: 2-chloro-5-fluoro-4-nitro-phenol

Ferric nitrate nonahydrate (14.06 g, 34.12 mmol) was added to a solution of 2-chloro-5-fluoro-phenol (5 g, 34.12 mmol) in and the resulting mixture was stirred at 55° C. for 5 hours. The mixture was then diluted with water and extracted with chloroform. The organic layer was concentrated and and crude product was recrystallized from toluene and heptane to afford 2 g (38%) of 2-chloro-5-fluoro-4-nitro-phenol. ¹H-NMR (400MHz, CDCl₃) δ (ppm): 8.19 (d, J=7.64 Hz, 1H), 6.93 (d, J=11.4 Hz, 1H), 6.42 (br s, 1H).

Step-2: 4-benzyloxy-5-Chloro-2-fluoro-nitrobenzene

K₂CO₃ (0.87 g, 6.26 mmol) and benzyl bromide (0.62 ml, 5.22 mmol) were added to a solution of 2-chloro-5-fluoro-4-nitro-phenol (1 g, 5.22 mmol) in DMF and the resulting mixture was stirred at room temperature for 2 hours. The reaction mixture was then diluted with water and extracted with ethyl acetate. The organic layer was concentrated and purified through column chromatography over Si-gel (1% EtOAC-hexane) to afford 1.1 g (76%) of 4-benzyloxy-5-Chloro-2-fluoro-nitrobenzene. GCMS [m/z]:281

Step-3: 5-Benzyloxy-4-chloro-2-nitrophenol

NaOH (475 mg, 11.1 mmol) was added to 4-benzyloxy-5-chloro-2-fluoro-nitrobenzene (220 mg, 0.79 mmol) in water and the solution was heated to reflux overnight. The reaction mixture was then diluted with water and adjusted to pH 3-4. The solution was extracted with ethyl acetate, which was washed with water and dried over sodium sulfate. Concentration of the organic layer afforded 100 mg (45%) of 5-benzyloxy-4-chloro-2-nitrophenol. GCMS [m/z]:279

Step-4: 2-Amino-5-benzyloxy-4-chlorophenol

SnCl₂.2H₂O (5.65 g, 25 mmol) was added to a solution of 5-benzyloxy-4-chloro-2-nitrophenol (1.4 g, 5.0 mmol) in ethanol and the resulting mixture ws heated to reflux for 5 hours. The mixture was basified and extracted with ethyl acetate. The organic layer was washed with water and dried over sodium sulfate. Concentration of organic layer afforded 975 mg (78%) of 2-amino-5-benzyloxy-4-chlorophenol. LC/MS [M+H]⁺: 250.4

Step-5: 7-Benzyloxy-6-chloro-3,4-dihydro-2H-benzo[1,4]oxazine-2-carboxylic acid ethyl ester

2,3-Dibromo-ethylpropiolate (0.67 ml, 4.68 mmol) and K₂CO₃ (1.5 g, 10.84 mmol) were added to 2-amino-5-benzyloxy-4-chlorophenol (970 mg, 3.9 mmol) in acetone and the resulting mixture was heated to reflux overnight. The reaction mixture was concentrated and purified through column chromatography over Si-gel (12% EtOAC-hexane) to afford 600 mg (45%) of 7-Benzyloxy-6-chloro-3,4-dihydro-2H-benzo[1,4]oxazine-2-carboxylic acid ethyl ester. LC/MS [M+H]⁺: 348.3

Step-6: 7-Benzyloxy-6-chloro-2,3-dihydro-benzo[1,4]oxazine-2,4-dicarboxylic acid 4-tert-butyl ester 2-ethyl ester

DMAP (21 mg, 0.17 mmol) and (Boc)₂O (3.8 ml, 17.3 mmol) were added to a solution of 7-Benzyloxy-6-chloro-3,4-dihydro-2H-benzo[1,4]oxazine-2-carboxylic acid ethyl ester (600 mg, 1.73 mmol) in THF and the resulting mixture was heated to reflux overnight. The mixture was then concentrated, diluted with water and extracted with ethyl acetate. The organic layer was dried over sodium sulfate and concentrated under reduced pressure. The crude product was purified through column chromatography over Si-gel (6%-ethyl acetate-hexane) to afford 1.7 g (75%) of 7-Benzyloxy-6-chloro-2,3-dihydro-benzo[1,4]oxazine-2,4-dicarboxylic acid 4-tert-butyl ester 2-ethyl ester. ¹H-NMR (400 MHz, CDCl₃) δ (ppm): 7.80 (br s, 1H), 7.45-7.28 (m, 5H), 6.63 (s, 1H), 5.08 (m, 2H0, 4.79 (t, 1H0, 4.22 (m, 3), 3.77 (m, 1H), 1.51 (s, 9H), 1.26 (t, 3H).

Step-7: 7-Benzyloxy-6-chloro-2,3-dihydro-benzo[1,4]oxazine-2,4-dicarboxylic acid 4-tert-butyl ester

Lithium hydroxide (150 mg, 3.57 mmol) was added to a solution of 7-Benzyloxy-6-chloro-2,3-dihydro-benzo[1,4]oxazine-2,4-dicarboxylic acid 4-tert-butyl ester 2-ethyl ester (800 mg, 1.79 mmol) in THF:H₂O (2:1) and the resulting mixture was stirred for 5-6 h, then concentrated. The crude product was neutralized with 50% aqueous acetic acid and extracted with ethyl acetate. Concentration of the ethyl acetate layer afforded 550 mg (76%) of 7-Benzyloxy-6-chloro-2,3-dihydro-benzo[1,4]oxazine-2,4-dicarboxylic acid 4-tert-butyl ester. ¹H-NMR (400 MHz, CDCl₃) δ (ppm): 7.80 (br s, 1H), 7.45-7.31 (m, 5H), 6.63 (s, 1H), 5.08 (m, 2H), 4.84 (m, 1H), 4.14 (m, 1H), 3.90 (m, 1H), 1.50 (s, 9H).

Step-8: 7-Benzyloxy-6-Chloro-2-[4-cyano-4-(4-fluoro-benzyl)-piperidine-1-carbonyl]-2,3-dihydro-benzo[1,4]oxazine-4-carboxylic acid tert-butyl ester

To a solution of ethyl-7-benzyloxy-6-chloro-2,3-dihydro-benzo[1,4]oxazine-4-carboxylic acid tert-butyl ester-2-carboxylic acid (550 mg, 1.3 mmol) in DMF was added 4-cyano-4-(4-fluoro-benzyl)-piperidine (286 mg, 1.31 mmol), EDCI (380 mg, 1.97 mmol), HOBt (212 mg, 1.57 mmol) and DIPEA (0.68 ml, 3.93 mmol) at 5-10° C. and the mixture was stirred at room temperature for 14-16 hours. The reaction mixture was then diluted with water and extracted with ethyl acetate. The organic layer was concentrated under reduced pressure and the residue was purified by flash chromatography (silica-gel, 2% MeOH-DCM) to afford 795 mg (98%) of 7-benzyloxy-6-chloro-2-[4-cyano-4-(4-fluoro-benzyl)-piperidine-1-carbonyl]-2,3-dihydro-benzo[1,4]oxazine-4-carboxylic acid tert-butyl ester.LC/MS [M+H]⁺: 620.5

Step-9: 6-Chloro-2-[4-cyano-4-(4-fluoro-benzyl)-piperidine-l-carbonyl]-7-hydroxy-2,3-dihydro-benzo[1,4]oxazine-4-carboxylic acid tert-butyl ester

7-benzyloxy-6-chloro-2[4-cyano-4-(4-fluoro-benzyl)-piperidine-1-carbonyl]-2,3-dihydro-benzo[1,4]oxazine-4-carboxylic acid tert-butyl ester (790 mg) was hydrogenated with 5% Pd-C (40 mg) in ethyl acetate for 2 h. The catalyst was removed by filtration through a celite bed. Concentration of the ethyl acetate solution yielded 650 mg (96.3%) of 6-chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester. LC/MS [M+H]⁺: 530.4

Step-10: 1-(6-Chloro-7-hydroxy-3,4-dihydro-2H-benzo[1,4]oxazine-2-carbonyl)-4-(4-fluoro-benzyl)-piperidine-4-carbonitrile

Trifluoroacetic acid (0.1 ml, 1.33 mmol) was added to 6-chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester (70 mg, 0.13 mmol) in DCM at 5-10° C. and the reaction mixture was stirred for 2-4 h at room temperature. The solution was then concentrated and neutralised with 1 (N) NaOH solution. The product was extracted with ethyl acetate and the organic layer was washed with water and brine solution. The organics were concentrated and purified through column chromatography over Si-gel (2% MeOH-DCM) to afford 20 mg (36%) of 1-(6-chloro-7-hydroxy-3,4-dihydro-2H-benzo[1,4]oxazine-2-carbonyl)-4-(4-fluoro-benzyl)-piperidine-4-carbonitrile. LC/MS [M+H]⁺: 430.2. ¹N-NMR (400 MHz, DMSO-d₆) δ (ppm): 9.16 (s, 1H), 7.34 (br s, 2H), 7.19 (m, 2H), 6.50 (s, 1H), 6.39 (s, 1H), 5.39 (s, 1H), 4.90 (br s, 1H), 4.38 (br s, 1H), 4.07 (br s, 1H), 3.17 (m, 2H), 2.96 (s, 2H), 2.67 (m, 1H), 1.81 (m, 3H), 1.54 (m, 1H). HPLC purity: 96.1%

### Example 38

### {6-Chloro-2-[4-cyano-4-(4-fluoro-benzyl)-piperidine-1-carbonyl]-3, 4-dihydro-2H-benzo[1,4]oxazin-7-yloxy}-acetic acid

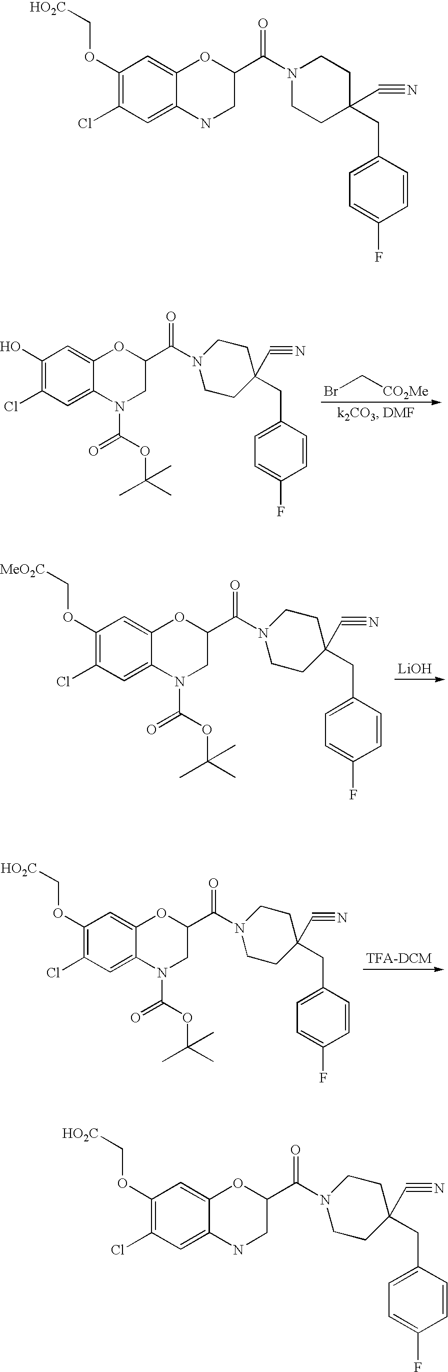

Step-1: 6-Chloro-2-[4-cyano-4-(4-fluoro-benzyl)-piperidine-1-carbonyl]-7-methoxycarbonylmethoxy-2,3-dihydro-benzo[1,4]oxazine-4-carboxylic acid tert-butyl ester

K₂CO₃ (156 mg, 1.13 mmol) and methyl bromoacetate (0.57 ml, 0.63 mmol) were added to 6-chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester (300 mg, 0.57 mmol) in DMF. The resulting mixture was stirred at room temperature for 3 hours then diluted with water. The resulting precipitate was filtered, washed with water and the residue was dissolved in ethyl acetate. The organic layer was concentrated to afford 315 mg (92.7%) of 6-Chloro-2-[4-cyano-4-(4-fluoro-benzyl)-piperidine-1-carbonyl]-7-methoxycarbonylmethoxy-2,3-dihydro-benzo[1,4]oxazine-4-carboxylic acid tert-butyl ester. LC/MS [M+H]⁺: 602.4

Step-2: 7-Carboxymethoxy-6-chloro-2-[4-cyano-4-(4-fluoro-benzyl)-piperidine-1-carbonyl]-2,3-dihydro-benzo[1,4]oxazine-4-carboxylic acid tert-butyl ester

Lithium hydroxide (20 mg, 0.37 mmol) was added to a solution of 6-Chloro-2-[4-cyano-4-(4-fluoro-benzyl)-piperidine-1-carbonyl]-7-methoxycarbonylmethoxy-2,3-dihydro-benzo[1,4]oxazine-4-carboxylic acid tert-butyl ester (90 mg, 0.15 mmol) in THF:H₂O (2:1) and the mixture was stirred for 5-6 h. After concentrating the mixture, the crude product was neutralized with 50% aqueous acetic acid and extracted with ethyl acetate. Concentration of the ethyl acetate layer afforded 65 mg (79%) of 7-Carboxymethoxy-6-chloro-2-[4-cyano-4-(4-fluoro-benzyl)-piperidine-1-carbonyl]-2,3-dihydro-benzo[1,4]oxazine-4-carboxylic acid tert-butyl ester. LC/MS [M+H]⁺: 588.2

Step-3: 2-{6-Chloro-2-[4-cyano-4-(4-fluoro-benzyl)-piperidine-1-carbonyl]-3,4-dihydro-2H-benzo[1,4]oxazin-7-yloxy}-acetic acid

Trifluoroacetic acid (0.1 ml, 1.33 mmol) was added to 7-Carboxymethoxy-6-chloro-2-[4-cyano-4-(4-fluoro-benzyl)-piperidine-1-carbonyl]-2,3-dihydro-benzo[1,4]oxazine-4-carboxylic acid tert-butyl ester (65 mg, 0.11 mmol) in DCM at 5-10° C. and the reaction mixture was stirred for 2-4 h at room temperature. The solution was concentrated and neutralised with 1 (N) NaOH solution. The product was extracted with ethyl acetate/EtOH and the organic layer was washed with water and brine solution. Concentration of the organic layer afforded 35 mg (65.2%) of 2-{6-chloro-2-[4-cyano-4-(4-fluoro-benzyl)-piperidine-1-carbonyl]-3,4-dihydro-2H-benzo[1,4]oxazin-7-yloxy}-acetic acid. LC/MS [M+H]⁺: 488.4. ¹H-NMR (400 MHz, DMSO-d₆) δ (ppm): 7.42-7.17 (m, 6H), 6.64 (s, 1H), 6.55 (m, 1H), 5.69 (br s, 1H), 4.97 (m, 1H), 4.36 (s, 2H), 4.03 (m, 2H), 3.21 (m, 2H), 2.95 (s, 2H), 2.67 (m, 1H), 1.80-1.54 (m, 4H). HPLC purity: 90.7%

### Example 39

### 2-{6-Chloro-2-[4-cyano-4-(4-fluoro-benzyl)-piperidine-1-carbonyl]-3,4-dihydro-2H-benzo[1,4]oxazin-7-yloxy}-acetamide

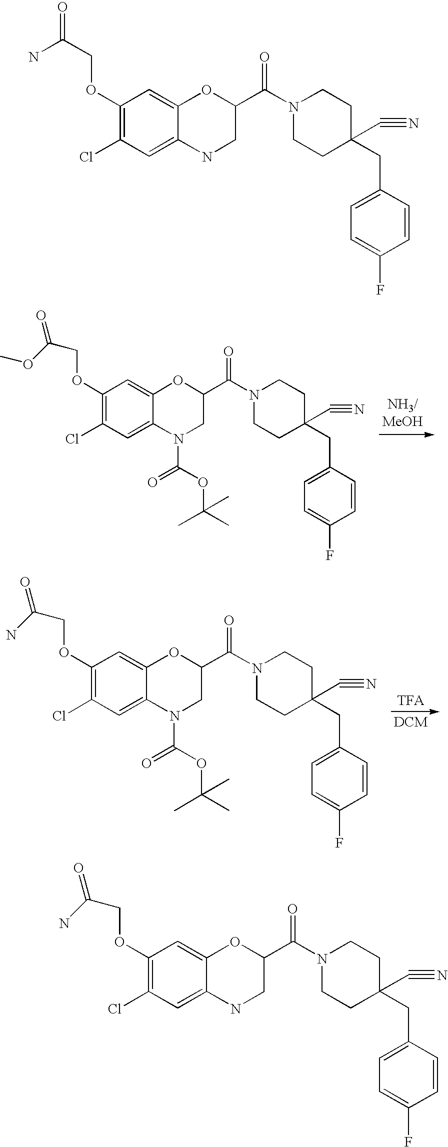

Step-1: 2-{6-Chloro-2-[4-cyano-4-(4-fluoro-benzyl)-piperidine-1-carbonyl]-3,4-dihydro-2H-benzo[1,4]oxazin-7-yloxy}-acetamide-4-carboxylic acid tert butyl ester

A solution of 6-Chloro-2-[4-cyano-4-(4-fluoro-benzyl)-piperidine-1-carbonyl]-7-methoxycarbonylmethoxy-2,3-dihydro-benzo[1,4]oxazine-4-carboxylic acid tert-butyl ester (100 mg) in methanol was saturated with ammonia by purging with ammonia gas at atmospheric pressure at −5° C. The resulting mixture was heated at 70° C. in a sealed tube overnight. The mixture was then concentrated to afford 70 mg (71.4%) of 2-{6-chloro-2-[4-cyano-4-(4-fluoro-benzyl)-piperidine-1-carbonyl]-3,4-dihydro-2H-benzo[1,4]oxazin-7-yloxy}-acetamide-4-carboxylic acid tert butyl ester. LC/MS [M+H]⁺: 587.2

Step-2: 2-{6-Chloro-2-[4-cyano-4-(4-fluoro-benzyl)-piperidine-1-carbonyl]-3,4-dihydro-2H-benzo[1,4]oxazin-7-yloxy}-acetamide

Trifluoroacetic acid (0.1 ml, 1.33 mmol) was added to 2-{6-chloro-2-[4-cyano-4-(4-fluoro-benzyl)-piperidine-1-carbonyl]-3,4-dihydro-2H-benzo[1,4]oxazin-7-yloxy}-acetamide-4-carboxylic acid tert butyl ester (50 mg, 0.085 mmol) in DCM at 5-10° C. and the resulting mixture was stirred for 2-4 h at room temperature. The solution was concentrated and neutralised with 1 (N) NaOH solution. The product was extracted with ethyl acetate/EtOH and the organic layer was washed with water and brine solution. Concentration of the organic layer afforded 30 mg (73.2%) of 2-{6-chloro-2-[4-cyano-4-(4-fluoro-benzyl)-piperidine-1-carbonyl]-3,4-dihydro-2H-benzo[1,4]oxazin-7-yloxy}-acetamide.LC/MS [M+H]⁺: 487.4. ¹H-NMR (400 MHz, DMSO-d₆) δ (ppm): 7.38-7.17 (m, 4H), 6.58 (s, 1H), 6.35 (s, 1H), 5.42 (s, 1H), 4.91 (br s, 1H), 4.38 (m, 1H), 4.09 (m, 2H), 4.02 (s, 2H), 3.19-2.60 (m, 6H), 1.78 (m, 2H). HPLC purity: 92.7%

### Example 40

### 2-{6-Chloro-2-[4-cyano-4-(4-fluoro-benzyl)-piperidine-1-carbonyl]-3,4-dihydro-2H-benzo[1,4]oxazin-7-yloxy}-N,N-dimethyl-acetamide

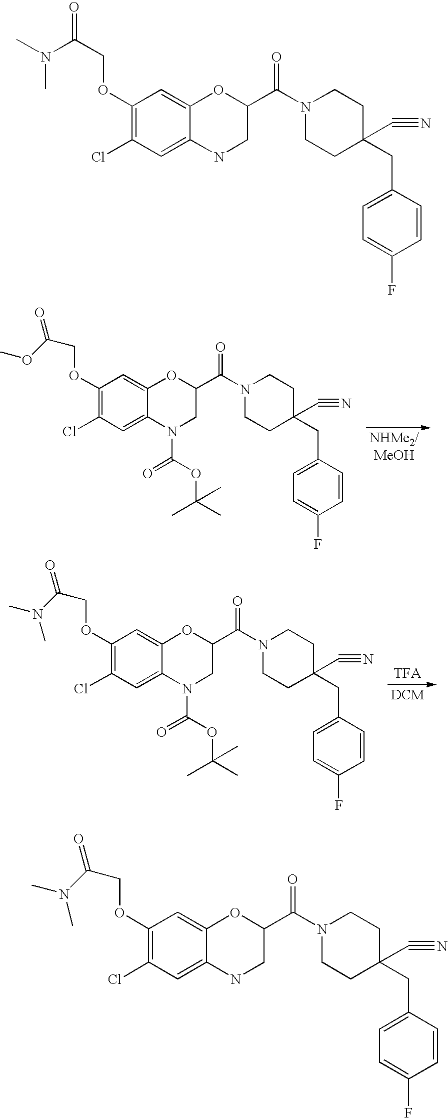

Step-1: 2-{6-Chloro-2-[4-cyano-4-(4-fluoro-benzyl)-piperidine-1-carbonyl]-3,4-dihydro-2H-benzo[1,4]oxazin-7-yloxy}-N,N-dimethyacetamide-4-carboxylic acid tert butyl ester

6-Chloro-2-[4-cyano-4-(4-fluoro-benzyl)-piperidine-1-carbonyl]-7-methoxycarbonylmethoxy-2,3-dihydro-benzo[1,4]oxazine-4-carboxylic acid tert-butyl ester (80 mg, 0.133 mmol) was heated in a THF solution of dimethylamine at 70° C. in a sealed tube overnight. Concentration of the reaction mixture afforded 80 mg (97.8%) of 2-16-chloro-2-[4-cyano-4-(4-fluoro-benzyl)-piperidine-1-carbonyl]-3,4-dihydro-2H-benzo[1,4]oxazin-7-yloxy}-N,N-dimethyacetamide-4-carboxylic acid tert butyl ester. LC/MS [M+H]⁺: 615.4

Step-2: 2-{6-Chloro-2-[4-cyano-4-(4-fluoro-benzyl)-piperidine-1-carbonyl]-3,4-dihydro-2H-benzo[1,4]oxazin-7-yloxy}-N,N-dimethyacetamide

Trifluoroacetic acid (0.1 ml, 1.33 mmol) was added to 2-{6-chloro-2-[4-eyano-4-(4-fluoro-benzyl)-piperidine-1-carbonyl]-3,4-dihydro-2H-benzo[1,4]oxazin-7-yloxy}-N,N-dimethyacetamide-4-carboxylic acid tert butyl ester (80 mg, 0.13 mmol) in DCM at 5-10° C. and the resulting mixture was stirred for 2-4 h at room temperature. The solution was concentrated and neutralised with 1 (N) NaOH solution. The product was extracted with ethyl acetate/EtOH and the organic layer was washed with water and brine solution. Concentration of the organic layer afforded 50 mg (74.7%) of 2-{6-chloro-2-[4-cyano-4-(4-fluoro-benzyl)-piperidine-1-carbonyl]-3,4-dihydro-2H-benzo[1,4]oxazin-7-yloxy}-N,N-dimethyacetamide. LC/MS [M+H]⁺: 515.4. ¹H-NMR (400 MHz, CDCl₃) δ (ppm): 7.30-6.55 (7H), 4.75 (br s, 1H), 4.66-3.32 (m, 8H), 3.09 (s, 3H), 2.96 (s, 3H), 2.86 (m, 3H), 1.90 (m, 2H), 1.73 (m, 1H).HPLC purity: 95.2%

### Example 41

### 1-(6-Chloro-7-ethoxy-3,4-dihydro-2H-benzo[1,4]oxazine-2-carbonyl)-4-(4-fluoro-benzyl)-piperidine-4-carbonitrile

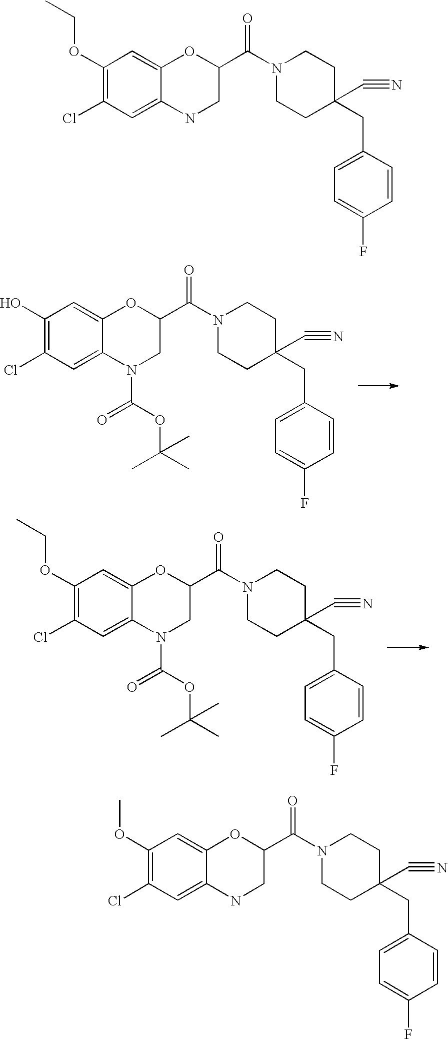

Step-1: 6-Chloro-2-[4-cyano-4-(4-fluoro-benzyl)-piperidine-1-carbonyl]-7-ethoxy-2,3-dihydro-benzo[1,4]oxazine-4-carboxylic acid tert-butyl ester

Anhydrous K₂CO₃ (52 mg, 0.38 mmol) and ethyl iodide (0.05 ml, 0.57 mmol) were added to 6-chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester (prepared as described in Example 37 (100 mg, 0.19 mmol) in acetone and the resulting mixture was stirred at 50° C. overnight. The mixture was then diluted with crushed ice and extracted with ethyl acetate. The organic layer was concentrated and purified through column chromatography over Si-gel (20% ethyl acetate-hexane) to afford 90 mg (85%) of 6-chloro-2-[4-cyano-4-(4-fluoro-benzyp-piperidine-1-carbonyl]-7-ethoxy-2,3-dihydro-benzo[1,4]oxazine-4-carboxylic acid tert-butyl ester. LC/MS [M+H]⁺:558.2

Step-2: 1-(6-Chloro-7-ethoxy-3,4-dihydro-2H-benzo[1,4]oxazine-2-carbonyl)-4-(4-fluoro-benzyl)-piperidine-4-carbonitrile

Trifluoroacetic acid (1 ml) was added to 6-chloro-2-[4-cyano-4-(4-fluoro-benzyl)-piperidine-1-carbonyl]-7-ethoxy-2,3-dihydro-benzo[1,4]oxazine-4-carboxylic acid tert-butyl ester (90 mg, 0.16 mmol) in DCM at 5-10° C. and the reaction mixture was stirred for 2-4 h at room temperature. The solution was then concentrated and neutralised with 1 (N) NaOH solution. The product was extracted with ethyl acetate/EtOH and the organic layer was washed with water and brine solution. Concentration of organic layer afforded 66 mg (75.8%) of 1-(6-chloro-7-ethoxy-3,4-dihydro-2H-benzo[1,4]oxazine-2-carbonyl)-4-(4-fluoro-benzyl)-piperidine-4-carbonitrile. LC/MS [M+H]⁺: 458.4. ¹N-NMR (400 MHz, DMSO-d₆) δ (ppm): 7.34-6.55 (6 Ar H), 5.59 (s, 1H), 4.97 (m, 1H), 4.38 (m, 1H), 4.07 (m, 1H), 3.94 (t, 2H), 3.19 (m, 2H), 2.95 (s, 2H), 2.69 (m, 1H), 1.89-1.39 (m, 4H), 1.27 (m, 4H). HPLC purity: 98.8%

### Example 42

### 1-[6-Chloro-4-(2-dimethylamino-acetyl)-7-methoxy-3,4-dihydro-2H-benzo[1,4]oxazine-2-carbonyl]-4-(4-fluoro-benzyl)-piperidine-4-carbonitrile

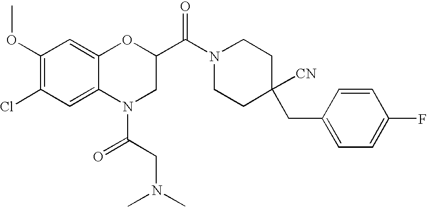

1-[6-Chloro-4-(2-dimethylamino-acetyl)-7-methoxy-3,4-dihydro-2H-1-benzo[1,4]oxazine-2-carbonyl]-4-(4-fluoro-benzyl)-piperidine-4-carbonitrile was prepared from 1-(6-Chloro-7-methoxy-3,4-dihydro-2H-benzo[1,4]oxazine-2-carbonyl)-4-(4-fluoro-benzyl)-piperidine-4-carbonitrile using a similar procedure to that described in Example 18.LC/MS [M+H]⁺: 529.3. ¹H-NMR (400 MHz, CDCl₃) δ (ppm): 8.04-6.69 (6 Ar H), 5.40 (m, 1H), 4.44-4.00 (m, 3H), 3.81 (s, 3H), 3.21-2.23 (m, 8H), 2.19 (s, 6H), 1.87-1.54 (m, 4H). HPLC purity: 92.4%

### Example 43

### 1-(4-Acetyl-6-chloro-7-methoxy-3,4-dihydro-2H-benzo[1,4]oxazine-2-carbonyl)-4-(4-fluoro-benzyl)-piperidine-4-carbonitrile

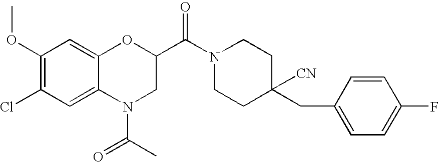

1-(4-Acetyl-6-chloro-7-methoxy-3,4-dihydro-2H-benzo[1,4]oxazine-2-carbonyl)-4-(4-fluoro-benzyl)-piperidine-4-carbonitrile was prepared from 1-(6-Chloro-7-methoxy-3,4-dihydro-2H-benzo[1,4]oxazine-2-carbonyl)-4-(4-fluoro-benzyl)-piperidine-4-carbonitrile using a similar procedure to that described in Example 15. LC/MS [M+H]⁺: 486.3. ¹H-NMR (400 MHz, CDCl₃) δ (ppm): 7.25-7.02 (m, 5H), 6.59 (s, 1H), 5.12 (s, 1H), 4.60 (d, J=14.4 Hz, 1H), 4.45 (m, 1H), 3.85 (s, 3H), 3.78 (m, 2H), 3.44 (m, 2H), 2.87 (m, 3H), 2.26 (s, 3H), 1.95 (m, 3H). HPLC purity: 95.1%

### Example 44

### 1-[4-(4-Chloro-benzyl)-4-hydroxy-3,3-dimethyl-piperidin-1-yl]-2-(6-chloro-3,4-dihydro-2H-benzo[1,4]oxazin-2-yl)-ethanone

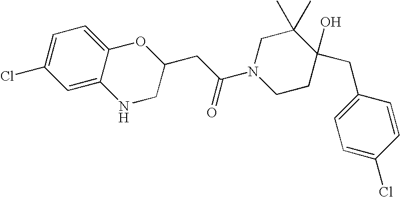

4-(4-Chloro-benzyl)-3,3-dimethyl-piperidin-4-ol was prepared as described in Example 32.

1-[4-(4-Chloro-benzyl)-4-hydroxy-3,3-dimethyl-piperidin-1-yl]-2-(6-chloro-3,4-dihydro-2H-benzo[1,4]oxazin-2-yl)-ethanone using a similar procedures to that described in Example 25. LC/MS [M+H]⁺: 463.3. ¹H-NMR (400 MHz, DMSO-d₆) δ (ppm): 7.31-7.24 (m, 4H), 6.61-6.43 (m, 3H), 6.08 (s, 1H), 4.36 (m, 1H), 4.21-2.61 (m, 10H), 1.60-1.23 (m, 2H), 0.92 (s, 6H). HPLC purity: 93.7%

### Example 45

### 4-(4-Chloro-benzyl)-1-[2-(6-chloro-3,4-dihydro-2H-benzo[1,4]oxazin-2-yl)-ethyl)-3,3-dimethyl-piperidin-4-ol

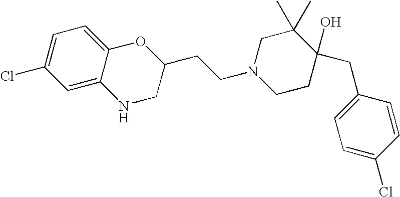

4-(4-Chloro-benzyl)-1-[2-(6-chloro-3,4-dihydro-2H-benzo[1,4]oxazin-2-yl)-ethyl]-3,3-dimethyl-piperidin-4-ol was prepared from 1-[4-(4-Chloro-benzyl)-4-hydroxy-3,3-dimethyl-piperidin-1-yl]-2-(6-chloro-3,4-dihydro-2H-benzo[1,4]oxazin-2-yl)-ethanone using a similar procedures to that described in Example 30. LC/MS [M+H]⁺: 449.3. ¹H-NMR (400 MHz, DMSO-d₆) δ (ppm): 7.29-7.23 (m, 4H), 6.62-6.41 (m, 3H), 6.08 (s, 1H), 3.98-1.51 (m, 16H), 1.02 (s, 3H), 0.90 (s, 3H). HPLC purity: 97.6%

### Example 45A

### 1-[2-(6-Chloro-3,4-dihydro-2H-benzo[1,4]oxazin-2-yl)-ethyl]-4-(4-fluoro-benzyl)-3,3-dimethyl-piperidin-4-ol

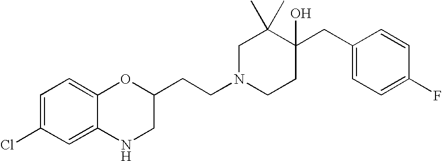

1-[2-(6-Chloro-3,4-dihydro-2H-benzo[1,4]oxazin-2-yl)-ethyl]-4-(4-fluoro-benzyl)-3,3-dimethyl-piperidin-4-ol was prepared using a similar procedure to that described in Example 45. LC/MS [M+H]⁺: 433.2. ¹H-NMR (400 MHz, DMSO-d₆) δ (ppm): 7.24 (m, 2H), 7.05 (m, 2H), 6.60 (d, 1H), 6.54 (m, 1H), 6.41 (d, 1H), 6.07 (br s, 1H), 3.99-1.68 (m, 16H), 1.03 (s, 3H), 0.90 (s, 3H). HPLC purity: 97.7%.

### Example 46

### 1-(6-Chloro-4-methanesulfonyl-7-methoxy-3,4-dihydro-2H-benzo[1,4]oxazine-2-carbonyl)-4-(4-fluoro-benzyl)-piperidine-4-carbonitrile

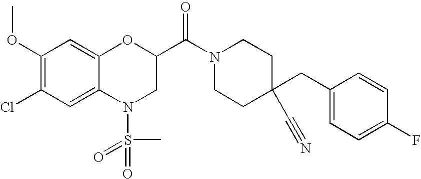

1-(6-Chloro-4-methanesulfonyl-7-methoxy-3,4-dihydro-2H-benzo[1,4]oxazine-2-carbonyl)-4-(4-fluoro-benzyl)-piperidine-4-carbonitrile was prepared from 1-(6-Chloro-7-methoxy-3,4-dihydro-2H-benzo[1,4]oxazine-2-carbonyl)-4-(4-fluoro-benzyl)-piperidine-4-carbonitrile using a similar procedures to that described in Example 16. LC/MS [M+H]⁺: 522.4. ¹H-NMR (400 MHz, CDCl₃) δ (ppm): 7.59-6.52 (6 Ar H), 4.95 (m, 1H), 4.65 (m, 1H), 4.10 (m, 3H), 3.86 (s, 3H), 3.60 (m, 1H), 3.49 (m, 3H), 2.96 (s, 3H), 1.96 (m, 3H), 1.67 (m, 1H). HPLC purity: 90.2%

### Example 47

### N-[7-(4-Chloro-benzyl)-piperidin-4-yl]-2-(6-chloro-3,4-dihydro-2H-benzo[1,4]oxazin-2-yl)-acetamide

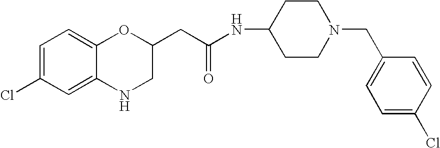

2-Carboxymethyl-6-chloro-2,3-dihydro-benzo[1,4]oxazine-4-carboxylic acid tert-butyl ester was prepared as described in Example 25.

1-(4-Chloro-benzyl)-piperidin-4-ylamine was prepared using a procedure similar to that described in Example 22.

N-[1-(4-Chloro-benzyl)-piperidin-4-yl]-2-(6-chloro-3,4-dihydro-2H-benzo[1,4]oxazin-2-yl)-acetamide was prepared from 1-(4-Chloro-benzyl)-piperidin-4-ylamine following amide coupling with 2-Carboxymethyl-6-chloro-2,3-dihydro-benzo[1,4]oxazine-4-carboxylic acid tert-butyl ester with 1-(4-Chloro-benzyl)-piperidin-4-ylamine followed by deboc with TFA using a procedure similar to that described in Example 25. LC/MS [M+H]⁺: 434.2. ¹H-NMR (400 MHz, DMSO-d₆) δ (ppm): 7.89 (d, 1H), 7.38-7.29 (m, 4H), 6.59-6.42 (m, 4H), 6.10 (s, 1H), 4.29 (m, 1H), 3.51 (m, 1H), 3.42 (s, 2H), 2.98 (m, 1H), 2.70 (m, 2H), 2.45-2.32 (m, 3H), 1.2.0 (t, 2H), 1.70 (m, 2H), 1.37 (m, 2H). HPLC purity: 99.1%

### Example 48

### [1-(4-Chloro-benzyl)-piperidin-4-yl]-[2-(6-chloro-3,4-dihydro-2H-benzo[1,4]oxazin-2-yl)-ethyl]-amine

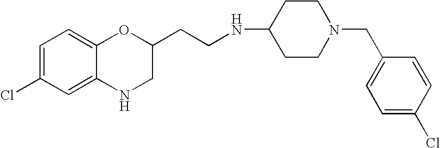

[1-(4-Chloro-benzyl)-piperidin-4-yl]-[2-(6-chloro-3,4-dihydro-2H-benzo[1,4]oxazin-2-yl)-ethyl]-amine was prepared from N-[1-(4-Chloro-benzyl)-piperidin-4-yl]-2-(6-chloro-3,4-dihydro-2H-benzo[1,4]oxazin-2-yl)-acetamide using a procedure similar to that described in Example 30. LC/MS [M+H]⁺: 420.4. ¹H-NMR (400 MHz, DMSO-d₆) δ (ppm): 7.36-7.28 (m, 4H), 6.61-6.54 (m, 2H), 6.42 (m, dd, 1H), 6.05 (s, 1H), 4.03 (m, 1H), 3.41 (s, 2H), 2.95 (m, 1H), 2.69 (m, 4H), 2.32 (m, 1H), 1.95 (t, 2H), 1.77-1.23 (m, 8H). HPLC purity: 97.6%

### Example 49

### 2-{6-Chloro-2-[4-cyano-4-(4-fluoro-benzyl)-piperidine-1-carbonyl]-7-methoxy-2,3-dihydro-benzo[1,4]oxazin-4-yl}-acetamide

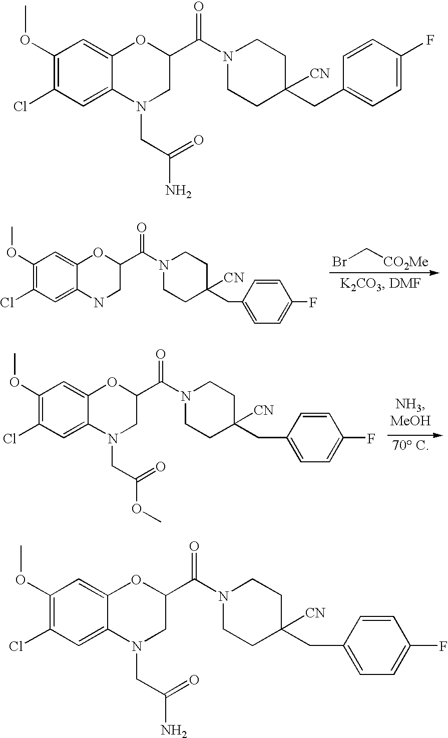

1-(6-Chloro-7-methoxy-3,4-dihydro-2H-benzo[1,4]oxazine-2-carbonyl)-4-(4-fluoro-benzyl)-piperidine-4-carbonitrile was prepared as described in Example 27.

Step-1: (6-Chloro-2-[4-cyano-4-(4-fluoro-benzyl)-piperidine-1-carbonyl]-7-methoxy-2,3-dihydro-benzo[1,4]oxazin-4-yl}-acetic acid methyl ester

Methyl bromoacetate (0.02 ml, 0.2 mmol) and K₂CO₃ (44.2 mg, 0.32 mmol) were added to a solution of 1-(6-chloro-7-methoxy-3,4-dihydro-2H-benzo[1,4]oxazine-2-carbonyl)-4-(4-fluoro-benzyl)-piperidine-4-carbonitrile (70 mg, 0.16 mmol) in DMF at 0° C. and the resulting mixture was stirred overnight at room temperature. The mixture was then diluted with ice-water and the resulting precipitate was filtered and dried to afford 65 mg (78.7%) of [6-chloro-2-[4-cyano-4-(4-fluoro-benzyl)-piperidine-1-carbonyl]-7-methoxy-2,3-dihydro-benzo[1,4]oxazin-4-yl}-acetic acid methyl ester. LC/MS [M+H]⁺: 516.0

Step-2: 2-{6-Chloro-2-[4-cyano-4-(4-fluoro-benzyl)-piperidine-1-carbonyl]-7-methoxy-2,3- dihydro-benzo[1,4]oxazin-4-yl}-acetamide

A solution of {6-chloro-2-[4-cyano-4-(4-fluoro-benzyl)-piperidine-1-carbonyl]-7-methoxy-2,3-dihydro-benzo[1,4]oxazin-4-yl}-acetic acid methyl ester in methanol was saturated with ammonia by purging with ammonia gas at atmospheric pressure at −5 ° C. The resulting mixture was heated overnight at 60° C. The mixture was then concentrated and washed with hexane to afford 30 mg (48%) of 2-{6-chloro-2-[4-cyano-4-(4-fluoro-benzyl)-piperidine-1-carbonyl]-7-methoxy-2,3-dihydro-benzo[1,4]oxazin-4-yl}-acetamide. LC/MS [M+H]⁺: 501.4. ¹H-NMR (400 MHz, CDCl₃) δ (ppm): 8.18 (br s, 1H), 8.05 (br s, 1H), 7.24 (t, 2H), 7.04 (t, 2H), 6.57 (d, 1H), 6.47 (s, 1H), 5.37 (s, 1H), 4.95 (d, 1H), 4.53 (d, 1H), 4.34 (d, 1H), 3.79 (s, 3H), 3.72-1.89 (m, 10H). HPLC purity: 98.3%

### Example 50

### (6-Chloro-7-methoxy-3,4-dihydro-2H-benzo[1,4]oxazin-2-yl)-[(R)-4-(4-fluoro-benzyl)-2-methyl-piperazin-1-yl]-methanone

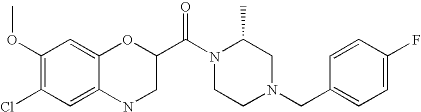

(R)-1-(4-fluoro-benzyl)-3-methyl-piperazine was prepared as described in Example 1.

6-Chloro-7-methoxy-3,4-dihydro-2H-benzo[1,4]oxazine-2-carboxylic acid was prepared as described in Example 27.

(6-Chloro-7-methoxy-3,4-dihydro-2H-benzo[1,4]oxazin-2-yl)-[(R)-4-(4-fluoro-benzyl)-2-methyl-piperazin-1-yl]-methanone was prepared from (R)-1-(4-fluoro-benzyl)-3-methyl-piperazine by amide coupling with 6-chloro-7-methoxy-3,4-dihydro-2H-benzo[1,4]oxazine-2-carboxylic acid using a procedure similar to that described in Example 27. LC/MS [M+H]⁺: 434.3 ¹H-NMR (400 MHz, CDCl₃) δ (ppm): 7.36 (t, 2H), 7.16 (t, 2H), 6.63-6.53 (m, 2H), 5.57 (br s, 1H), 4.88 (m, 1H), 4.49 (br s, 1H), 4.17 (m, 1H), 3.80 (m, 1H), 3.69 (s, 3H), 3.52-1.98 (m, 7H), 1.16 (d, 3H). HPLC purity: 99.5%.

### Example 51

### 1-(6-Chloro-7-dimethylamino-3,4-dihydro-2H-benzo[1,4]oxazine-2-carbonyl)-4-(4-fluoro-benzyl)-piperidine-4-carbonitrile

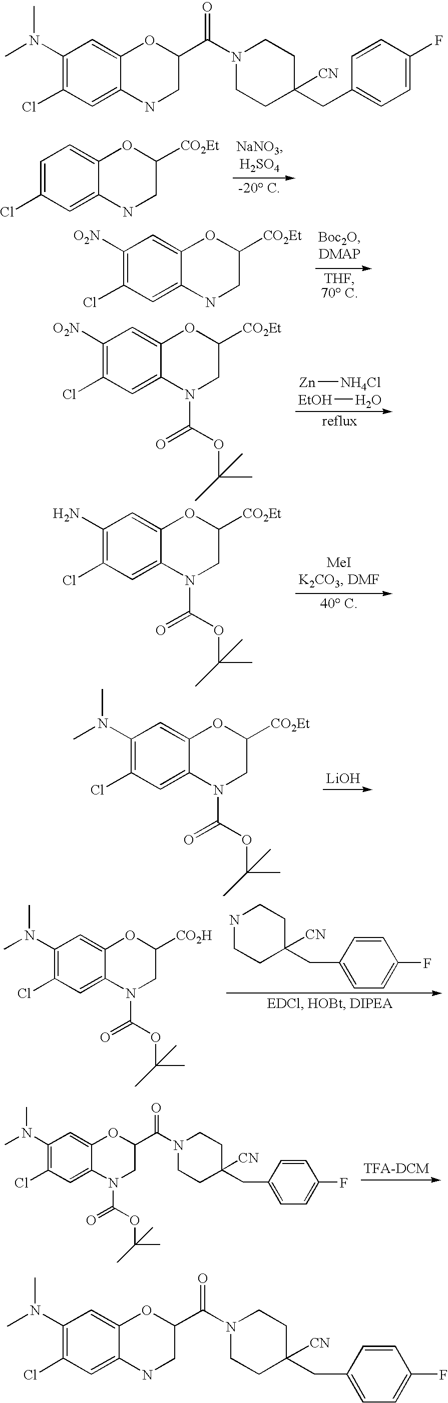

4-Cyano-4-(4-fluoro-benzyl)-piperidine was prepared as described above in Example 23.

Step-1: 6-Chloro-7-nitro-3,4-dihydro-2H-benzo[1,4]oxazine-2-carboxylic acid ethyl ester

NaNO₃ (0.53 g, 6.2 mmol) was added to a solution of 6-chloro-3,4-dihydro-2H-benzo[1,4]oxazine-2-carboxylic acid ethyl ester (1 g, 4.1 mmol) in H₂SO₄ at −30° C. and the mixture was stirred at −30° C. for 2 hour. The mixture was then poured into ice-water and extracted with ethyl acetate. The organic layer was washed with water and dried over sodium sulfate. The organic layer was then concentrated and purified through Si-gel column chromatography (7% ethyl acetate-hexane) to afford 350 mg (30%) of 6-chloro-7-nitro-3,4-dihydro-2H-benzo[1,4]oxazine-2-carboxylic acid ethyl ester. GCMS [m/z]:286.0

Step-2: 6-Chloro-7-nitro-2,3-dihydro-benzo[1,4]oxazine-2,4-dicarboxylic acid 4-tert-butyl ester 2-ethyl ester

DMAP (5 mg) and Boc₂O (0.1 ml, 0.35 mmol) were added to a solution of 6-chloro-7-nitro-3,4-dihydro-2H-benzo[1,4]oxazine-2-carboxylic acid ethyl ester (100 mg, 0.35 mmol) in THF and the reaction mixture was refluxed for 3 hours. After concentrating the mixture, the crude product was purified through Si-gel column chromatography (3% ethyl acetate-hexane) to afford 100 mg (74%) of 6-chloro-7-nitro-2,3-dihydro-benzo[1,4]oxazine-2,4-dicarboxylic acid 4-tert-butyl ester 2-ethyl ester. ¹H-NMR (400 MHz, CDCl₃) δ (ppm): 8.04 (s, 1H), 7.66 (s, 1H), 4.91 (t, 1H), 4.48 (dd, 1H), 4.21 (m, 2H), 3.71 (dd, 1H), 1.27 (t, 3H).

Step-3: 7-Amino-6-chloro-2,3-dihydro-benzo[1,4]oxazine-2,4-dicarboxylic acid 4-tert-butyl ester 2-ethyl ester

Zn-powder (710 mg, 10.85 mmol) and NH₄Cl (248 mg, 4.65 mmol) were added to a solution of 6-chloro-7-nitro-2,3-dihydro-benzo[1,4]oxazine-2,4-dicarboxylic acid 4-tert-butyl ester 2-ethyl ester (600 mg, 1.55 mmol) in ethanol-water (4:1) and the mixture was heated to reflux for for 4 hours. The reaction mixture was then filtered through celite bed. The organic layer was concentrated, diluted with water and the product was extracted with ethyl acetate. The organic layer was washed with water, dried over sodium sulfate and concentrated to afford 450 mg (81.5%) of 7-amino-6-chloro-2,3-dihydro-benzo[1,4]oxazine-2,4-dicarboxylic acid 4-tert-butyl ester 2-ethyl ester. LC/MS [M+H]⁺: 357.2

Step-4: 6-Chloro-7-dimethylamino-2,3-dihydro-benzo[1,4]oxazine-2,4-dicarboxylic acid 4-tert-butyl ester 2-ethyl ester

K₂CO₃ (232 mg, 1.68 mmol) and methyl iodide (0.3 ml) were added to a solution of 7-amino-6-chloro-2,3-dihydro-benzo[1,4]oxazine-2,4-dicarboxylic acid 4-tert-butyl ester 2-ethyl ester (150 mg, 0.42 mmol) in DMF. The reaction mixture was heated for 3 hour at 40° C. then diluted with water and extracted with ethyl acetate. The organic layer was washed with water and dried over sodium sulfate. The reaction mixture was concentrated and purified through Si-gel column chromatography (6% ethyl acetate-hexane) to afford 45 mg (28%) of 6-chloro-7-dimethylamino-2,3-dihydro-benzo[1,4]oxazine-2,4-dicarboxylic acid 4-tert-butyl ester 2-ethyl ester. LC/MS [M+H]⁺: 385.2

Step-5: 6-Chloro-7-dimethylamino-2,3-dihydro-benzo[1,4]oxazine-2,4-dicarboxylic acid 4-tert-butyl ester

LiOH (10 mg, 0.2 mmol) was added to a solution of 6-chloro-7-dimethylamino-2,3-dihydro-benzo[1,4]oxazine-2,4-dicarboxylic acid 4-tert-butyl ester 2-ethyl ester (70 mg, 0.18 mmol) in THF: H₂O (2:1) and the resuling solution was stirred for 3 hours. The reaction mixture was concentrated and the crude product was neutralized with 6(N) HCl solution and extracted with ethyl acetate. Concentration of the ethyl acetate layer afforded 55 mg (85.6%) of 6-chloro-7-dimethylamino-2,3-dihydro-benzo[1,4]oxazine-2,4-dicarboxylic acid 4-tert-butyl ester. LC/MS [M+H]⁺: 357.4

Step-6: 6-Chloro-2-[4-cyano-4-(4-fluoro-benzyl)-piperidine-1-carbonyl]-7-dimethylamino-2,3-dihydro-benzo[1,4]oxazine-4-carboxylic acid tent-butyl ester

To a solution 6-chloro-7-dimethylamino-2,3-dihydro-benzo[1,4]oxazine-2,4-dicarboxylic acid 4-tert-butyl ester (55 mg, 0.15 mmol) in DMF was added 4-(4-Fluoro-benzyl)-piperidine-4-carbonitrile (33.6 mg, 0.15 mmol)), EDCI (38.4 mg, 0.20 mmol), HOBt (10 mg, 0.07 mmol) and DIPEA (01 ml, 0.4 mmol) at 5-10 ° C. and the mixture was stirred at room temperature for 14-16 h. The mixture was then diluted with water and extracted with ethyl acetate. The ethyl acetate layer was concentrated in vacuo and subjected to column chromatography (Si-gel, 1% MeOH-DCM) to afford 35 mg (41%) 6-chloro-2-[4-cyano-4-(4-fluoro-benzyp-piperidine-1-carbonyl]-7-dimethylamino-2,3-dihydro-benzo[1,4]oxazine-4-carboxylic acid tert-butyl ester. LC/MS [M+H]⁺: 557.4

Step-7: 1-(6-Chloro-7-dimethylamino-3,4-dihydro-2H-benzo[1,4]oxazine-2-carbonyl)-4-(4-fluoro-benzyl)-piperidine-4-carbonitrile

Trifluoroacetic acid (0.5 ml) was added to a solution of 6-chloro-2-[4-cyano-4-(4-fluoro-benzyl)-piperidine-1-carbonyl]-7-dimethylamino-2,3-dihydro-benzo[1,4]oxazine-4-carboxylic acid tert-butyl ester (35 mg, 0.06 mmol) in DCM and the resulting mixture was stirred for 5 h. The mixture was then concentrated and washed with dry ether. The TFA salt was diluted with a minimum volume of water and neutralized with aq. NaOH solution at 5-10° C. The reaction mixture was extracted with ethyl acetate and washed with water and brine solution. The organic layer was dried over sodium sulfate and concentrated under reduced pressure to afford 18 mg (62.7%) of 1-(6-Chloro-7-dimethylamino-3,4-dihydro-2H-benzo[1,4]oxazine-2-carbonyl)-4-(4-fluoro-benzyl)-piperidine-4-carbonitrile. LC/MS [M+H]⁺: 457.4. ¹H-NMR (400 MHz, DMSO-d₆) δ (ppm): 7.34 (br s, 2H), 7.19 (m, 2H), 6.56 (br d, 2H), 4.92 (m, 1H), 4.38 (m, 1H), 4.03 (m, 1H), 3.19 (m, 2H), 2.88 (br s, 2H), 2.68 (m, 1H), 2.57 (s, 6H), 1.81-1.54 (m, 5H). HPLC purity: 89.1%

### Example 52

### N-{6-Chloro-2-[4-cyano-4-(4-fluoro-benzyl)-piperidine-1-carbonyl]-3,4-dihydro-2H-benzo[1,4]oxazin-7-yl}-acetamide

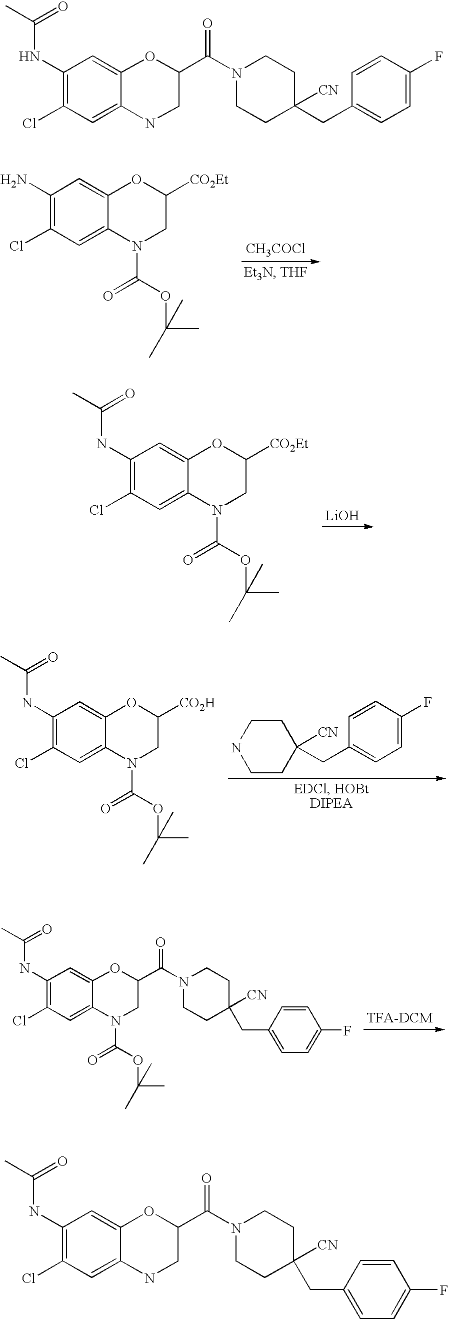

4-Cyano-4-(4-fluoro-benzyl)-piperidine was prepared as described above in Example 23.

7-Amino-6-chloro-2,3-dihydro-benzo[1,4]oxazine-2,4-dicarboxylic acid 4-tert-butyl ester 2-ethyl ester was prepared as described in Example 51.

Step 1: 7-Acetylamino-6-chloro-2,3-dihydro-benzo[1,4]oxazine-2,4-dicarboxylic acid 4-tent-butyl ester 2-ethyl ester

Triethyl amine (0.13 ml, 0.91 mmol) and acetyl chloride (0.03 ml, 0.4 mmol) were added to a solution of 7-amino-6-chloro-2,3-dihydro-benzo[1,4]oxazine-2,4-dicarboxylic acid 4-tert-butyl ester 2-ethyl ester (130 mg, 0.36 mmol) in THF at 10° C. and the reaction mixture was stirred overnight. The mixture was concentrated, diluted with water and extracted with ethyl acetate. The organic layer was washed with water, dried over sodium sulfate, concentrated and purified through Si-gel column chromatography (6% ethyl acetate-hexane) to afford 60 mg (41%) of 7-acetylamino-6-chloro-2,3-dihydro-benzo[1,4]oxazine-2,4-dicarboxylic acid 4-tert-butyl ester 2-ethyl ester. LC/MS [M+H]⁺: 399.2, 416.6 (M +17)

Step-2: 7-Acetylamino-6-chloro-2,3-dihydro-benzo[1,4]oxazine-2,4-dicarboxylic acid 4-tert-butyl ester

LiOH (6 mg, 0.15 mmol) was added to a solution of 7-acetylamino-6-chloro-2,3-dihydro-benzo[1,4]oxazine-2,4-dicarboxylic acid 4-tert-butyl ester 2-ethyl ester (60 mg, 0.15 mmol) in THF:H₂O (2:1) and the mixture was stirred for 3 hours. The mixture was then concentrated and the crude product was neutralized with 50% acetic acid and extracted with ethyl acetate. Concentration of the ethyl acetate layer afforded 35 mg (63%) of 7-acetylamino-6-chloro-2,3-dihydro-benzo[1,4]oxazine-2,4-dicarboxylic acid 4-tert-butyl ester. LC/MS [M+H]⁺: 371.4

Step-3: 7-Acetylamino-6-chloro-2-[4-cyano-4-(4-fluoro-benzyl)-piperidine-1-carbonyl]-2,3-dihydro-benzo[1,4]oxazine-4-carboxylic acid tent-butyl ester

To a solution 7-acetylamino-6-chloro-2,3-dihydro-benzo[1,4]oxazine-2,4-dicarboxylic acid 4-tert-butyl ester (35 mg, 0.094 mmol) in DMF was added 4-(4-fluoro-benzyl)-piperidine-4-carbonitrile (21 mg, 0.094 mmol)), EDCI (27 mg, 0.14 mmol), HOBt (16 mg, 0.112 mmol) and DIPEA (0.05 ml, 0.28 mmol) at 5-10° C. and the mixture was stirred at room temperature for 14-16 h. The mixture was then diluted with water and extracted with ethyl acetate. The ethyl acetate layer was concentrated in vacuo and subjected to column chromatography (Si-gel, 35% ethyl acetate-hexane) to afford 30mg (56%) 7-acetylamino-6-chloro-2-[4-cyano-4-(4-fluoro-benzyl)-piperidine-1-carbonyl]-2,3-dihydro-benzo[1,4]oxazine-4-carboxylic acid tert-butyl ester. LC/MS [M+H]⁺: 571.6

Step-4: N-{6-Chloro-2-[4-cyano-4-(4-fluoro-benzyl)-piperidine-1-carbonyl]-3,4-dihydro-2H-benzo[1,4]oxazin-7-yl}-acetamide

Trifluoroacetic acid (0.5 ml) was added to a solution of 7-acetylamino-6-chloro-2-[4-cyano-4-(4-fluoro-benzyl)-piperidine-1-carbonyl]-2,3-dihydro-benzo[1,4]oxazine-4-carboxylic acid tert-butyl ester (30 mg, 0.05 mmol) in DCM and the resulting mixture was stirred for 5 h. The mixture was then concentrated and washed with dry ether. The TFA salt was diluted with a minimum volume of water and neutralized with aq. NaOH solution at 5-10° C. Extraction of the neutralized solution with ethyl acetate followed by concentration afforded 18 mg (73.5%) of N-{6-chloro-2-[4-cyano-4-(4-fluoro-benzyl)-piperidine-1-carbonyl]-3,4-dihydro-2H-benzo[1,4]oxazin-7-yl}-acetamide. LC/MS [M+H]⁺: 471.4. ¹H-NMR (400 MHz, CDCl3) δ (ppm): 7.81-6.65 (m, 7H), 4.77 (br s, 1H), 4.57 (m, 1H), 4.25 (m, 1H), 3.66 (m, 1H), 3.45 (m, 1H), 3.28 (m, 1H), 3.86 (br s, 2H), 2.19 (s, 3H), 2.03-1.40 (m, 6H). HPLC purity: 99.6%

### Example 53

### (6-Chloro-7-methoxy-4-methyl-3,4-dihydro-2H-benzo[1,4]oxazin-2-yl)-[(R)-4-(4-fluoro-benzyl)-2-methyl-piperazin-1-yl]-methanone

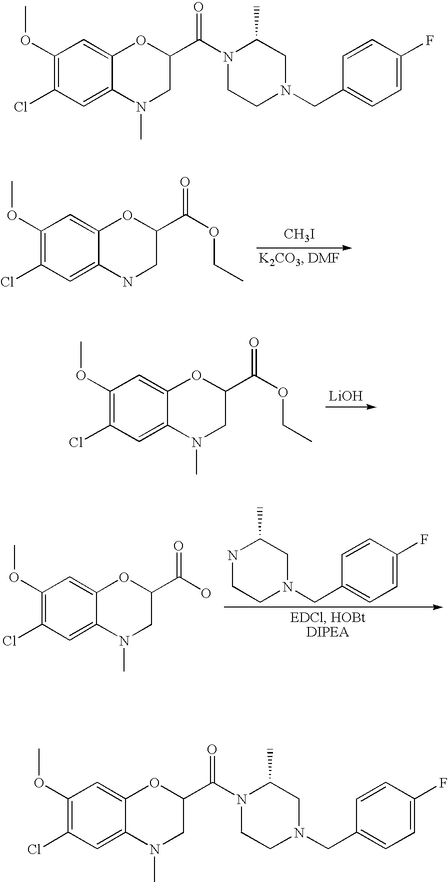

Step-1: 6-Chloro-7-methoxy-4-methyl-3,4-dihydro-2H-benzo[1,4]oxazine-2-carboxylic acid ethyl ester

To a solution of 6-Chloro-7-methoxy-3,4-dihydro-2H-benzo[1,4]oxazine-2-carboxylic acid ethyl ester (300 mg, 1.1 mmol) in DMF was added K₂CO₃ (470 mg, 3.3 mmol) and methyl iodide (0.2 ml, 3.3 mmol) at 0° C. The reaction mixture was stirred at 40 ° C. for 2 and half hours and diluted with water. The organics was extracted with ethyl acetate, washed with water and dried over anhd. Na₂SO₄. The organic layer was concentrated and purified over column chromatography (Si-gel, 7% ethyl acetate-hexane) to afford 167 mg (53%) of 6-Chloro-7-methoxy-4-methyl-3,4-dihydro-2H-benzo[1,4]oxazine-2-carboxylic acid ethyl ester. LC/MS [M+H]⁺: 286.2

Step-2: 6-Chloro-7-methoxy-4-methyl-3, 4-dihydro-2H-benzo[1,4]oxazine-2-carboxlic acid

To a solution of 6-Chloro-7-methoxy-4-methyl-3,4-dihydro-2H-benzo[1,4]oxazine-2-carboxylic acid ethyl ester (165 mg, 0.58 mmol) in THF:H₂O (2:1) was added lithium hydroxide (48 mg, 1.15 mmol). The reaction mixture was stirred for 5-6 h at rt and concentrated. The crude mass was neutralized with 50% aqueous acetic acid and extracted with ethyl acetate. Concentration of the ethyl acetate layer afforded 110 mg (73.6%) of 6-Chloro-7-methoxy-4-methyl-3,4-dihydro-2H-benzo[1,4]oxazine-2-carboxylic acid. LC/MS [M+H]⁺: 258.4

Step-3: (6-Chloro-7-methoxy-4-methyl-3,4-dihydro-2H-benzo[1,4]oxazin-2-yl)-[(R)-4-(4-fluoro-benzyl)-2-methyl-piperazin-1-yl]-methanone

To a solution of 6-Chloro-7-methoxy-4-methyl-3,4-dihydro-2H-benzo[1,4]oxazine-2-carboxylic acid (50 mg, 0.19 mmol) in DMF was added (R)-1-(4-Fluoro-benzyl)-3-methyl-piperazine (40.4 mg, 0.19 mmol), EDCI (47.3 mg,0.25 mmol 1), HOBt (12.8 mg, 0.095 mmol) and DIPEA (0.096 ml, 0.57 mmol) at 5-10° C. The reaction mixture was stirred at room temperature for 15 hours, diluted with water and extracted with ethyl acetate. The ethyl acetate layer was concentrated in vacuo and subjected to column chromatography (Si-gel, 3% MeOH-DCM) to afford 35 mg (40%) of (6-Chloro-7-methoxy-4-methyl-3,4-dihydro-2H-benzo[1,4]oxazin-2-yl)-[(R)-4-(4-fluoro-benzyl)-2-methyl-piperazin-1-yl]-methanone. LC/MS [M+H]⁺: 448.4. ¹H-NMR (400 MHz, DMSO-d₆) δ (ppm):7.28 (m, 2H), 7.00 (m, 2H), 6.68 (s, 1H), 6.47 (s, 1H), 4.87 (brs, 1H), 4.69 (brs, 1H), 4.37 (m, 1H), 3.78 (s, 3H), 3.52-3.28 (m, 4H), 2.86 (s, 4H), 2.68-2.41 (m, 5H), 1.28 (m,3H). HPLC purity: 99.3%

### Example 54

### 6-Chloro-7-methoxy-3,4-dihydro-2H-benzo[1,4]oxazine-2-carboxylic acid [1-(4-fluoro-benzyl)-piperidin-4-yl]-amide

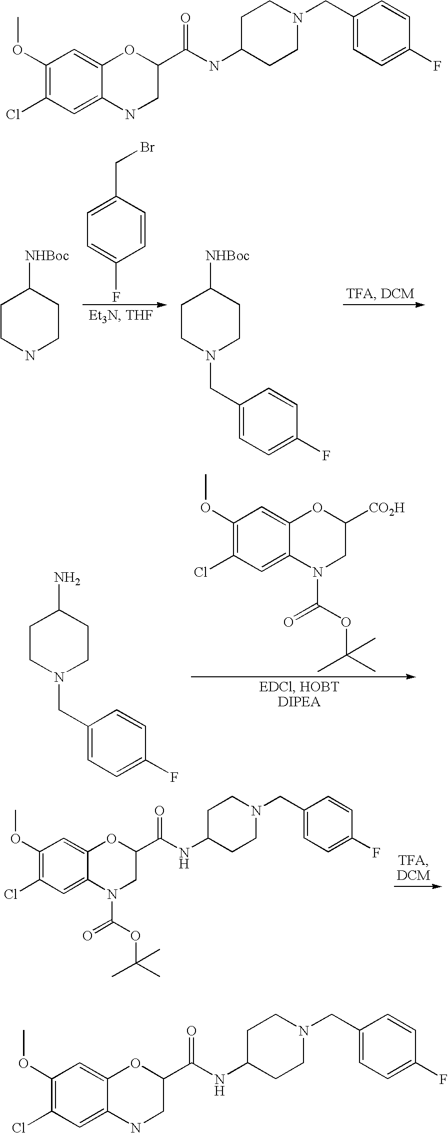

Step-1: [1-(4-Fluoro-benzyl)-piperidin-4-yl]-carbamic acid tert-butyl ester

To a solution of Piperidin-4-yl-carbamic acid tert-butyl ester (1 g, 4.9 mmol) in THF was added triethyl amine (1.3 ml, 9.8 mmol) and 4-fluorobenzyl bromide (0.79 ml, 6.3 mmol) at ice-cold condition. The reaction mixture was stirred at room temperature for 12 hrs and concentrated The reaction mass was diluted with water and extracted with ethyl acetate. The organic layer was washed with water, brine and dried over Na₂SO₄. The organic layer was concentrated under reduced pressure and washed with hexane to afford 500 mg (86.5%) of [1-(4-Fluoro-benzyl)-piperidin-4-yl]-carbamic acid tert-butyl ester.

LC/MS [M+H]⁺: 309

Step-2: 1-(4-Fluoro-benzyl)-piperidin-4-ylamine

To a solution [1-(4-Fluoro-benzyl)-piperidin-4-yl]-carbamic acid tert-butyl ester (1.4 g, 4.5 mmol) in DCM was added trifluoroacetic acid (0.27 ml,3.61 mmol) drop wise at cold condition. The mixture was allowed to stir at room temperature for 3 hours. Then reaction mixture was concentrated under reduced pressure. The crude mass was diluted with water, basified with 2N NaOH solution and extracted with ethyl acetate. The organic layer was washed with water and brine, then dried over Na₂SO₄. Concentration of the organic layer under reduced pressure afforded 700 mg (74.7%) of 1-(4-Fluoro-benzyl)-piperidin-4-ylamine.LC/MS [M+H]⁺: 209.4

Step-3: 6-Chloro-2-[1-(4-fluoro-benzyl)-piperidin-4-ylcarbamoyl]-7-methoxy-2,3-dihydro-benzo[1,4]oxazine-4-carboxylic acid tent-butyl ester

To a solution of 6-Chloro-7-methoxy-2, 3-dihydro-benzo[1,4]oxazine-2, 4-dicarboxylic acid-4-tert-butyl ester (100 mg, 0.29 mmol) in DMF was added EDCI (72.49 mg, 0.37 mmol), HOBT (19.6 mg, 0.14 mmol) and DIPEA (0.16 ml, 0.87 mmol) at 0° C. The resulting mixture was allowed to stir at room temperature for 30 minutes. Then a solution of 1-(4-Fluoro-benzyl)-piperidin-4-ylamine in DMF was added to the previous solution at 5° C. The reaction mixture was allowed to stir at rt overnight. The reaction mixture was diluted with water and extracted with ethyl acetate. The organic layer was concentrated under reduced pressure and the crude was purified by column chromatography (Si-gel, 0.5% MeOH-DCM) to afford 60 mg (38.7%) of 6-Chloro-2-[1-(4-fluoro-benzyl)-piperidin-4-ylcarbamoyl]-7-methoxy-2, 3-dihydro-benzo[1,4]oxazine-4-carboxylic acid tert-butyl ester. LC/MS [M+H]⁺:534.4

Step-4: 6-Chloro-7-methoxy-3, 4-dihydro-2H-benzo[1, 4]oxazine-2-carboxylic acid [1-(4-fluoro-benzyl)-piperidin-4-yl]-amide

To a solution of 6-Chloro-2-[1-(4-fluoro-benzyl)-piperidin-4-ylcarbamoyl]-7-methoxy-2,3-dihydro-benzo[1,4]oxazine-4-carboxylic acid tert-butyl ester (50 mg, 0.09 mmol) in DCM was added trifluoroacetic acid (0.04 ml, 0.744 mmol) drop wise under ice-cold condition. The reaction mixture was allowed to stir at room temperature for 3 hours. The reaction mixture was concentrated and the crude mass was diluted with water, basified with 2N NaOH solution and extracted with ethyl acetate. Concentration of organic layer under reduced pressure afforded 9 mg (22.3%) of 6-Chloro-7-methoxy-3, 4-dihydro-2H-benzo[1,4]oxazine-2-carboxylic acid [1-(4-fluoro-benzyl)-piperidin-4-yl]-amide. LC/MS [M+H]⁺: 434.4. ¹H-NMR (400 MHz, DMSO-d₆): δ (ppm) 7.77 (d, 1H), 7.30 (m, 2H), 7.13 (t, 2H), 6.86 (m, 2H), 5.60 (s, 1H), 4.44 (d, 1H), 3.68 (s, 3H), 3.59 (bs, 1H), 3.41 (s, 3H), 3.11 (m, 1H), 2.88 (m, 1H), 2.00 (m, 2H), 1.67 (m, 2H), 1.52 (m, 2H), 1.23 (bs, 1H). HPLC purity: 96.8%

### Example 55

### 4-(4-Fluoro-benzyl)-1-(7-methoxy-3,4-dihydro-2H-benzo[1,4]oxazine-2-carbonyl)-piperidine-4-carbonitrile

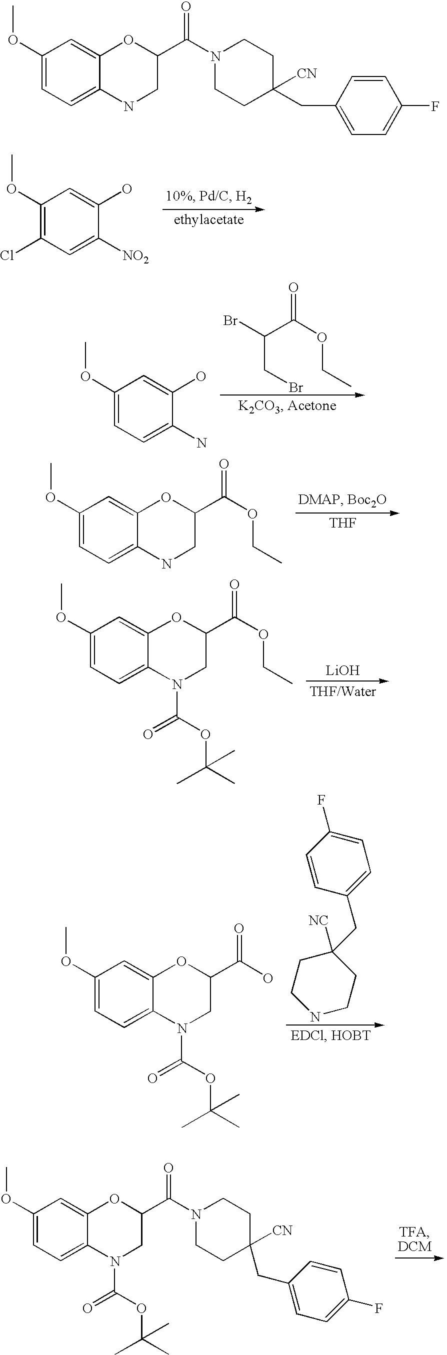

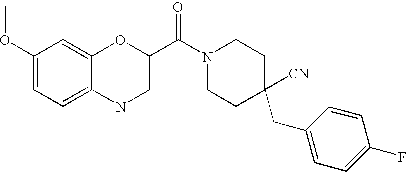

Step-1: 2-Amino-5-methoxy-phenol

A solution of 4-Chloro-5-methoxy-2-nitro-phenol (500 mg, 2.4 mmol) in ethyl acetate was hydrogenated overnight in presence of 10% Pd—C (50 mg). The catalyst was filtered off and ethyl acetate solution was concentrated. The crude solid was washed with ether and hexane to afford 260 mg (77.8%) of 2-amino-5-methoxyphenol. LC/MS [M+H]⁺: 140.

Step-2: 7-Methoxy-3,4-dihydro-2H-benzo[1,4]oxazine-2-carboxylic acid ethyl ester

To a solution of 2-amino-5-methoxyphenol (260 mg,1.86 mmol) in acetone was added 2,3-dibromopropiobnate (0.28 ml,1.97 mmol) drop wise and heated the reaction mixture with potassium carbonate (692.7 mg,5.01 mmol) for 20 hours. The reaction mixture was concentrated under reduced pressure and the crude was purified over column chromatography (Si-gel, 6% EtOAC-hexane) to afford 120 mg (27%) of 7-Methoxy-3,4-dihydro-2H-benzo[1,4]oxazine-2-carboxylic acid ethyl ester. GCMS (m/z): 237

Step-3: 7-Methoxy-2,3-dihydro-benzo[1,4]oxazine-2,4-dicarboxylic acid 4-tert-butyl ester 2-ethyl ester

To a solution of 7-Methoxy-3, 4-dihydro-2H-benzo[1, 4]oxazine-2-carboxylic acid ethyl ester (120 mg, 0.51 mmol) in THF was added catalytic amount (6.23 mg, 0.015 mmol) of DMAP and stirred the solution for 5 hrs. The THF solution was concentrated under reduced pressure and the crude was purified over column chromatography (Si-gel, 6% EtOAC-hexane) to afford 100 mg (51%) of 7-Methoxy-2, 3-dihydro-benzo[1,4]oxazine-2, 4-dicarboxylic acid 4-tert-butyl ester 2-ethyl ester.

¹H-NMR (400 MHz, DMSO-d₆): δ (ppm) 7.08-6.54 (m, 3Ar—H), 4.21 (m, 1H), 3.75 (s, 3H), 1.51 (m, 3H), 1.39 (m, 9H).

Step-4: 7-Methoxy-2,3-dihydro-benzo[1,4]oxazine-2,4-dicarboxylic acid 4-tert-butyl ester

To a solution of 7-Methoxy-2, 3-dihydro-benzo[1,4]oxazine-2, 4-dicarboxylic acid 4-tert-butyl ester 2-ethyl ester (90 mg, 0.27 mmol) in THF-water (3:1) was added LiOH.H₂O (22.4 mg, 0.53 mmol) in cold condition. The mixture was allowed to stir at room temperature for one and half hour. The reaction mixture was concentrated, diluted with water, neutralized with AcOH and extracted with ethyl acetate. The organic layer was washed with water and brine, then dried over Na₂SO₄ and concentrated under reduced pressure. The crude mass was washed with hexane to afford 50 mg (60%) of solid 7-Methoxy-2, 3-dihydro-benzo[1,4]oxazine-2, 4-dicarboxylic acid 4-tert-butyl ester.

Step-5: 2-[4-Cyano-4-(4-fluoro-benzyl)-piperidine-1-carbonyl]-7-methoxy-2,3-dihydro-benzo[1,4]oxazine-4-carboxylic acid tert-butyl ester

To a solution of 7-Methoxy-2,3-dihydro-benzo[1,4]oxazine-2,4-dicarboxylic acid 4-tert-butyl ester (40 mg, 0.13 mmol) in DMF was added EDCI (32.4 mg, 0.17 mmol), HOBT (8.8 mg, 0.065 mmol) and DIPEA (0.03 ml, 0.39 mmol) at 10° C. The resulting mixture was allowed to stir at room temperature for 30 minutes. Then a solution of 4-(4-Fluoro-benzyl)-piperidine-4-carbonitrile (28.4 mg, 0.13 mmol) in DMF was added to the previous reaction mixture at 10° C. and the reaction mixture was allowed to stir at room temperature overnight. The reaction mass was diluted with ice water, stirred for 10 minutes and extracted with ethyl acetate. The organic layer was washed with water and brine, dried over Na₂SO₄ and concentrated under reduced pressure. The crude organic mass was purified over column chromatography (Si-gel, 6% EtOAC-hexane) to afford 30 mg (44.6%) of 2-[4-Cyano-4-(4-fluoro-benzyl)-piperidine-1-carbonyl]-7-methoxy-2, 3-dihydro-benzo[1,4]oxazine-4-carboxylic acid tert-butyl ester. LC/MS [M+H]⁺: 510.4

Step-6: 4-(4-Fluoro-benzyl)-1-(7-methoxy-3,4-dihydro-2H-benzo[1,4]oxazine-2-carbonyl)-piperidine-4-carbonitrile

To a solution of 2-[4-Cyano-4-(4-fluoro-benzyl)-piperidine-1-carbonyl]-7-methoxy-2,3-dihydro-benzo[1,4]oxazine-4-carboxylic acid tert-butyl ester (30 mg, 0.053 mmol) in DCM was added trifluoroacetic acid (0.1 ml) drop wise at cold condition. The mixture was allowed to stir at room temperature for one and half an hour. The reaction mixture was evaporated and the crude mass was diluted with water, basified with 2N NaOH solution and extracted with ethyl acetate. The organic layer was washed with water and dried over Na₂SO₄. Concentration of the organic extract under reduced pressure afforded 4 mg (18%) of 4-(4-Fluoro-benzyl)-1(7-methoxy-3,4-dihydro-2H-benzo[1,4]oxazine-2-carbonyl)-piperidine-4-carbonitrile. LC/MS [M+H]⁺: 410.2.HPLC purity: 87%

### Example 56

### 6-Chloro-2-[4-cyano-4-(4-fluoro-benzyl)-piperidine-1-carbonyl]-7-methoxy-2,3-dihydro-benzo[1,4]oxazine-4-carboxylic acid amide

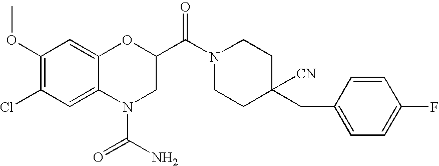

6-Chloro-2-[4-cyano-4-(4-fluoro-benzyl)-piperidine-1-carbonyl]-7-methoxy-2,3-dihydro-benzo[1,4]oxazine-4-carboxylic acid amide was prepared from 1-(6-Chloro-7-methoxy-3,4-dihydro-2H-benzo[1,4]oxazine-2-carbonyl)-4-(4-fluoro-benzyl)-piperidine-4-carbonitrile by a process similar to that described in example 11.LC/MS [M+H]⁺: 487.3.

### Example 57

### 1-(7-Bromo-6-chloro-3,4-dihydro-2H-benzo[1,4]oxazine-2-carbonyl)-4-(4-fluoro-benzyl)-piperidine-4-carbonitrile

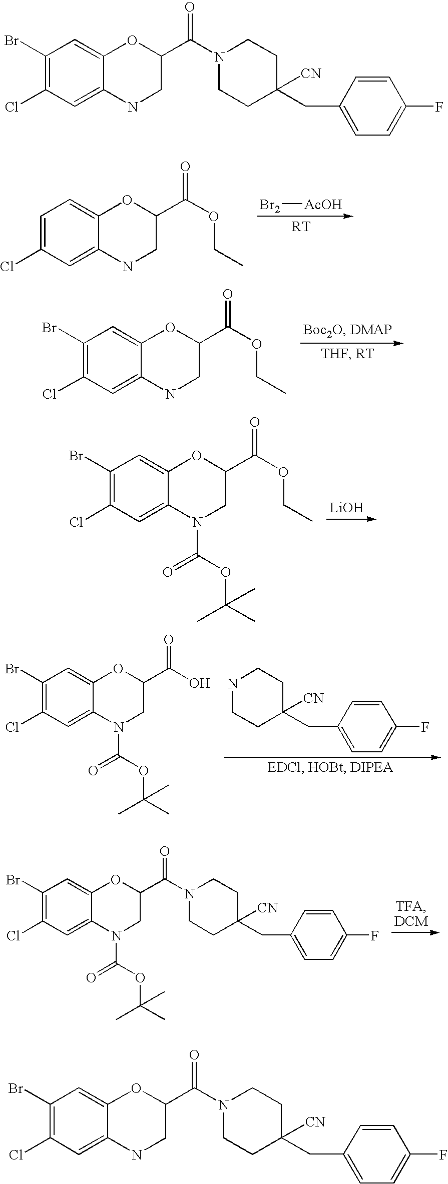

Step-1: 7-Bromo-6-chloro-3,4-dihydro-2H-benzo[1,4]oxazine-2-carboxylic acid ethyl ester

To a stirred solution of 6-Chloro-3,4-dihydro-2H-benzo[1,4]oxazine-2-carboxylic acid ethyl ester (41.38 m.mol,10.0 g) in acetic acid was added Br₂ (41.38 m.mol,2.13 ml) drop wise at 10° C. the reaction mixture was stirred for 30 min at same temperature. The reaction mixture was poured in ice-water, extracted the compound with ethyl acetate, washed with water, brine, dried over Na₂SO4 and concentrated under reduced pressure. Thus obtained crude was purified through column chromatography (Si-gel, 20% DCM-Hex) to afford 6.3 g (47.6%) of 7-Bromo-6-chloro-3, 4-dihydro-2H-benzo[1,4]oxazine-2-carboxylic acid ethyl ester. ¹H-NMR (400 MHz, CDCl₃): δ(ppm) 7.13 (s, 1H), 6.67 (s, 1H), 4.77 (t, 1H), 4.22 (q, 2H), 3.85 (s, 1H), 3.56 (d, 2H), 1.26 (t, 3H).

Step-2: 7-Bromo-6-chloro-2,3-dihydro-benzo[1,4]oxazine-2,4-dicarboxylic acid 4-tent-butyl ester 2-ethyl ester

To a stirred solution of 7-Bromo-6-chloro-3, 4-dihydro-2H-benzo[1,4]oxazine-2-carboxylic acid ethyl ester (4.5 g, 14.06 m.mol) in THF (30 ml), DMAP (0.172 g, 1.4 m.mol) and (BOC)₂O (6.46 ml, 28.13 m.mol) were added and stirred the reaction mixture overnight at room temperature. The reaction mixture was concentrated and purified through column chromatography (Si-gel, 3% EA/Hex) to afford 5.0 g (84.5%) of 7-Bromo-6-chloro-2,3-dihydro-benzo[1,4]oxazine-2,4-dicarboxylic acid 4-tert-butyl ester 2-ethyl ester. ¹H-NMR (400 MHz, CDCl₃): δ(ppm) 7.87-6.92 (m, 2H), 4.83 (t, 1H), 4.34 (dd, 1H), 4.21 (m, 2H), 3.70 (dd, 1H), 1.51 (s, 9H), 1.27 (t, 3H).

Step-3: 7-Bromo-6-chloro-2,3-dihydro-benzo[1,4]oxazine-2,4-dicarboxylic acid-4-tert-butyl ester

To a stirred solution of 7-Bromo-6-chloro-2,3-dihydro-benzo[1,4]oxazine-2,4-dicarboxylic acid 4-tert-butyl ester 2-ethyl ester(11.91 m.mol,5.0 g) in THF:Water (3:1) was added LiOH (23.81 m.mol, 1.0 g) and stirred the reaction mixture for 2 h at room temperature. The reaction mixture was concentrated, diluted with water, acidified with dilute acetic acid and extracted the organics with ethyl acetate. The organic layer was washed with water, brine, dried over Na₂SO4 and concentrated under reduced pressure. Washed the crude with hexane to afford 4.1 g (87.7%) of 7-Bromo-6-chloro-2,3-dihydro-benzo[1,4]oxazine-2,4-dicarboxylic acid 4-tert-butyl ester. ¹H-NMR (400 MHz, CDCl₃): δ(ppm) 7.9-6.89 (m, 3H), 4.87-3.77 (m, 3H), 1.5 (s, 9H).

Step-4: 7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester

To a solution of 7-Bromo-6-chloro-2, 3-dihydro-benzo[1,4]oxazine-2, 4-dicarboxylic acid 4-tert-butyl ester (4.1 g, 10.5 m.mol) in DMF was added 4-(4-Fluoro-benzyl)-piperidine-4-carbonitrile (2.28 g, 10.5 m.mol,), EDCI (3.0 g, 15.7 m.mol), HOBt (1.69 g, 12.6 m.mol) and DIPEA (5.4 ml, 31.4 m.mol) at 5-10° C. The reaction mixture was stirred at room temperature for 15 hours then diluted with water. The precipitated solid was filtered and dried to afford 5.4 g (87.1%) of 7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester. LCMS (M+H): 594.0. ¹H-NMR (400 MHz, CDCl₃): δ(ppm) 8.0-7.0 5 (m, 6H), 4.94-3.38 (m, 8H), 2.9 (s, 2H), 2.87 (m, 1H) 2.06-1.93 (m, 2H), 1.2 (s, 9H).

Step-5: 1-(7-Bromo-6-chloro-3,4-dihydro-2H-benzo[1,4]oxazine-2-carbonyl)-4-(4-fluoro-benzyl)-piperidine-4-carbonitrile

To a solution of 7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester (60 mg, 0.1 mmol) in DCM was added trifluoroacetic acid (0.3 ml, 3.9 mmol). The reaction mixture was stirred for about 5 h, concentrated and washed with dry ether. The resulting TFA salt was diluted with a minimum volume of water and neutralized with aq. NaOH solution at 5-10° C. Extraction of the neutralized solution with ethyl acetate followed by concentration under reduced pressure afforded 35 mg (71%) of 1-(7-Bromo-6-chloro-3,4-dihydro-2H-benzo[1,4]oxazine-2-carbonyl)-4-(4-fluoro-benzyl)-piperidine-4-carbonitrile. LC/MS [M+H]⁺: 493.3. ¹H-NMR (400 MHz, CDCl₃): δ(ppm) 7.28 (m, 1H), 7.25 (m, 1H), 7.05 (m, 3H), 6.72 (m, 1H), 4.74 (m, 1H), 4.62 (m, 1H), 4.18 (m, 1H), 3.97 (m, 1H), 3.59 (m, 3H), 3.33 (m, 1H), 2.89 (m, 3H), 1.94-1.1.67 (m, 2H). HPLC purity: 90.5%.

### Example 58

### 1-(6-Chloro-8-dimethylamino-3,4-dihydro-2H-benzo[1,4]oxazine-2-carbonyl)-4-(4-fluoro-benzyl)-piperidine-4-carbonitrile

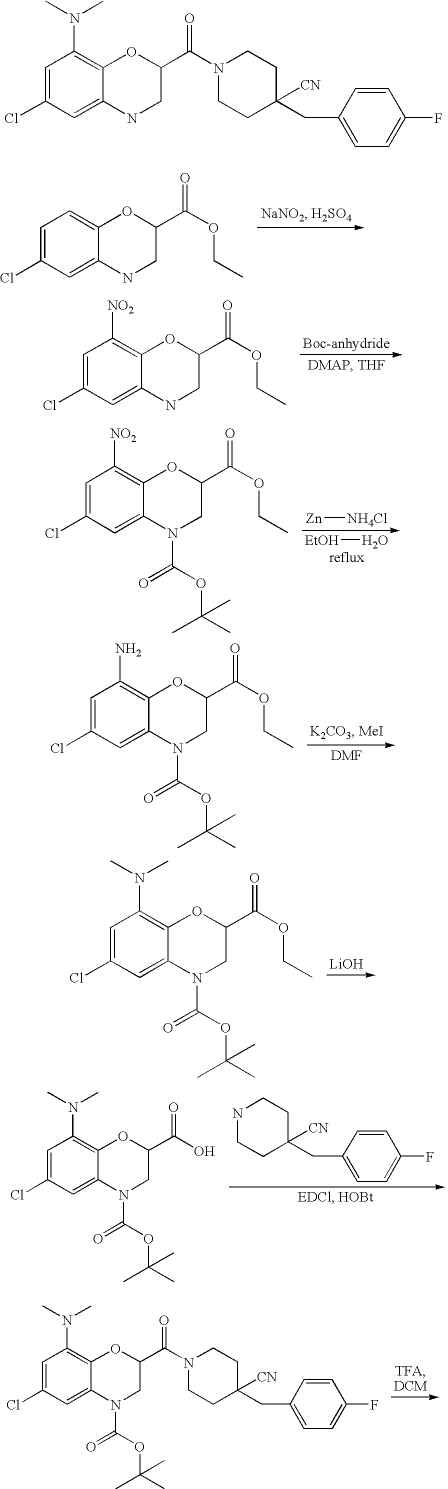

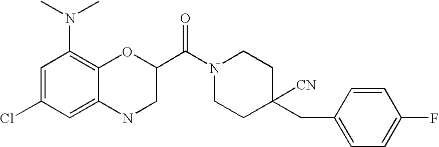

Step-1: 6-Chloro-8-nitro-3,4-dihydro-2H-benzo[1,4]oxazine-2-carboxylic acid ethyl ester

NaNO₃ (0.53 g, 6.2 mmol) was added to a solution of 6-chloro-3,4-dihydro-2H-benzo[1,4]oxazine-2-carboxylic acid ethyl ester (1 g, 4.1 mmol) in H₂SO₄ at −30° C. and the mixture was stirred at −30° C. for 2 hour. The mixture was then poured into ice-water and extracted with ethyl acetate. The organic layer was washed with water, dried over sodium sulfate and the organic layer was concentrated under reduced pressure and purified through Si-gel column chromatography (5% ethyl acetate-hexane) to afford 400 mg (28.2%) of 6-Chloro-8-nitro-3,4-dihydro-2H-benzo[1,4]oxazine-2-carboxylic acid ethyl ester. GCMS (m/z): 286.0

1-(6-Chloro-8-dimethylamino-3,4-dihydro-2H-benzo[1,4]oxazine-2-carbonyl)-4-(4-fluoro-benzyl)-piperidine-4-carbonitrile was prepared by a similar method as described in example 51. LC/MS [M+H]⁺: 457.0. ¹H-NMR (400 MHz, DMSO-d₆): δ(ppm) 7.22 (m, 2H), 7.03 (t, 2H), 6.29 (m, 2H), 4.67 (m, 2H), 4.32 (d, 1H), 3.85 (bs, 1H), 3.50 (m, 4H), 3.32 (m, 111), 2.89 (m, 1H), 2.73 (s, 6H), 1.92 (m, 2H), 1.75 (m, 1H), 0.86 (m, 1H). HPLC purity: 77%.

### Example 59:

### 1-(6,7-Dichloro-3,4-dihydro-2H-benzo[1,4]oxazine-2-carbonyl)-4-(4-fluoro-benzyl)-piperidine-4-carbonitrile

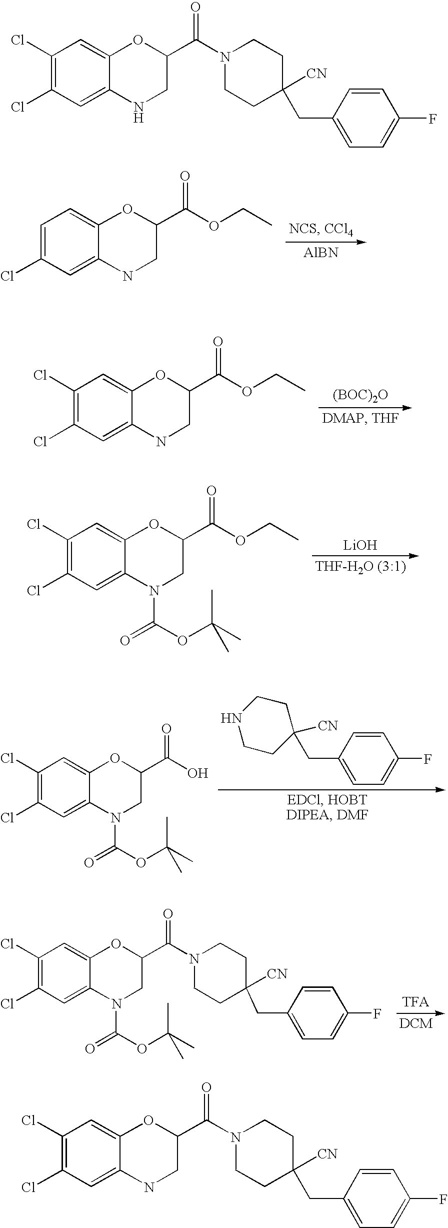

Step-1: 6,7-Dichloro-3,4-dihydro-2H-benzo[1,4]oxazine-2-carboxylic acid ethyl ester

To a solution of 6-Chloro-3,4-dihydro-2H-benzo[1,4]oxazine-2-carboxylic acid ethyl ester (200 mg, 0.83 mmol) in CCl₄ was added N-Chloro succinimide (116 mg,0.87 mmol) and AIBN (20 mg) at 0° C. The resulting mixture was allowed to stir at room temperature for 30 minutes and then refluxed for 3 hours. Then organic solution was evaporated under reduced pressure. The crude was purified over column chromatography (Si-gel, 2% ethyl acetate-hexane) to afford 50 mg (22%) of 6,7-Dichloro-3,4-dihydro-2H-benzo[1,4]oxazine-2-carboxylic acid ethyl ester. ¹H-NMR (400 MHz, CDCl₃): δ(ppm) 6.99 (s, 1H), 6.65 (s, 1H), 4.78 (t, 1H), 4.24 (m, 2H), 3.82 (s, 1H), 3.56 (s, 1H), 3.31 (t, 1H).

Step-2: 6,7-Dichloro-2,3-dihydro-benzo[1,4]oxazine-2,4-dicarboxylic acid 4-tert-butyl ester 2-ethyl ester

To a solution of 6,7-Dichloro-3,4-dihydro-2H-benzo[1,4]oxazine-2-carboxylic acid ethyl ester (150 mg,0.54 mmol) in THF was added DMAP (6.6 mg, 0.05 mmol) and di-tert-butyl carbonate (0.62 ml, 2.72 mmol). The mixture was allowed to reflux at 80° C. overnight. The reaction mixture was evaporated under reduced pressure. The crude was purified over column chromatography (Si-gel, 2% ethyl acetate-hexane) to afford 130 mg (64%) of 6,7-Dichloro-2,3-dihydro-benzo[1,4]oxazine-2,4-dicarboxylic acid 4-tert-butyl ester 2-ethyl ester. ¹H-NMR (400 MHz, DMSO-d₆): δ(ppm) 7.89 (s, 1H), 7.30 (s, 1H), 5.27 (bs, 1H), 4.46 (d, 1H), 4.12 (m, 1H), 3.57 (d, 1H), 1.46 (s, 9H).

Step-3: 6,7-Dichloro-2,3-dihydro-benzo[1,4]oxazine-2,4-dicarboxylic acid 4-tert-butyl ester

To a solution of 6, 7-Dichloro-2, 3-dihydro-benzo[1,4]oxazine-2, 4-dicarboxylic acid 4-tert-butyl ester 2-ethyl ester (130 mg,0.35 mmol) in THF-water (3:1) was added LiOH.H₂O (17.4 mg, 0.41 mmol) in ice-cold condition. The mixture was allowed to stir at room temperature during one and half hour. The reaction mixture was evaporated and the crude mass was diluted with water, neutralized with AcOH and extracted with ethyl acetate. The organic layer was washed with water, brine and concentrated under reduced pressure. The crude mass was washed with hexane to afford 80 mg (66%) of 6, 7-Dichloro-2, 3-dihydro-benzo[1,4]oxazine-2,4-dicarboxylic acid 4-tert-butyl ester. ¹H-NMR (400 MHz, DMSO-d₆): δ(ppm) 7.91 (bs, 1H), 7.26 (m, 1H), 7.16 (m, 1H), 5.07 (bs, 1H), 4.39 (d, 1H), 3.58 (d, 1H), 1.45 (s, 9H).

Step-4: 6,7-Dichloro-2-[4-cyano-4-(4-fluoro-benzyl)-piperidine-1-carbonyl]-2,3-dihydro-benzo[1,4]oxazine-4-carboxylic acid tert-butyl ester

To a solution of 6,7-Dichloro-2,3-dihydro-benzo[1,4]oxazine-2,4-dicarboxylic acid 4-tert-butyl ester (80 mg, 0.23 mmol) in DMF was added 4-cyano-4-(4-fluoro-benzyl)-piperidine (286 mg, 1.31 mmol), EDCI (57.3 mg, 0.29 mmol), HOBT (15.5 mg, 0.11 mmol) and DIPEA (0.12 ml, 0.69 mmol) at 5-10° C. and the mixture was stirred at room temperature for 14-16 hours. The reaction mixture was then diluted with water and extracted with ethyl acetate. The organic layer was concentrated under reduced pressure and the residue was purified by flash chromatography (silica-gel, 2% MeOH-DCM) to afford 300 mg (59%) of 6,7-Dichloro-2-[4-cyano-4-(4-fluoro-benzyl)-piperidine-1-carbonyl]-2,3-dihydro-benzo[1,4]oxazine-4-carboxylic acid tert-butyl ester. ¹H-NMR (400 MHz, DMSO-d₆): δ(ppm) 7.85 (s, 1H), 7.33 (m, 2H), 7.20 (m, 3H), 5.49 (m, 1H), 4.33 (d, 1H), 4.15 (m, 2H), 3.62 (m, 1H), 3.17 (m, 1H), 2.96 (s, 2H), 2.67 (m, 1H), 1.85 (m, 3H), 1.56 (m, 1H), 1.46 (s, 9H), 1.12 (s, 1H). LC/MS [M+H]⁺: 547.8

Step-5: 1-(6,7-Dichloro-3,4-dihydro-2H-benzo[1,4]oxazine-2-carbonyl)-4-(4-fluoro-benzyl)-piperidine-4-carbonitrile

Trifluoroacetic acid (0.1 ml, 1.33 mmol) was added to 6,7-Dichloro-2-[4-cyano-4-(4-fluoro-benzyl)-piperidine-1-carbonyl]-2,3-dihydro-benzo[1,4]oxazine-4-carboxylic acid tert-butyl ester (70 mg, 0.13 mmol) in DCM at 5-10° C. and the reaction mixture was stirred for 2-4 h at room temperature. The solution was then concentrated and neutralised with 1 (N) NaOH solution. The product was extracted with ethyl acetate and the organic layer was washed with water and brine solution. The organics were concentrated under reduced pressure to afford 55 mg (96%) of 1-(6,7-Dichloro-3,4-dihydro-2H-benzo[1,4]oxazine-2-carbonyl)-4-(4-fluoro-benzyl)-piperidine-4-carbonitrile. LC/MS [M+H]⁺: 447.9. ‘H-NMR (400 MHz, DMSO-d₆) δ(ppm): 7.34 (s, 2H), 7.19 (t, 2H), 6.94 (m, 1H), 6.75 (s, 1H), 5.01 (bs, 1H), 4.37 (bs, 1H), 4.05 (m, 1H), 3.39 (m, 1H), 2.95 (s, 1H), 2.69 (m, 1H), 1.79 (m, 3H), 1.54 (m, 1H), 1.23 (s, 1H). HPLC purity: 95.9%.

### Example 60

### 1-(6-Chloro-7-isopropoxy-3,4-dihydro-2H-benzo[1,4]oxazine-2-carbonyl)-4-(4-fluoro-benzyl)-piperidine-4-carbonitrile

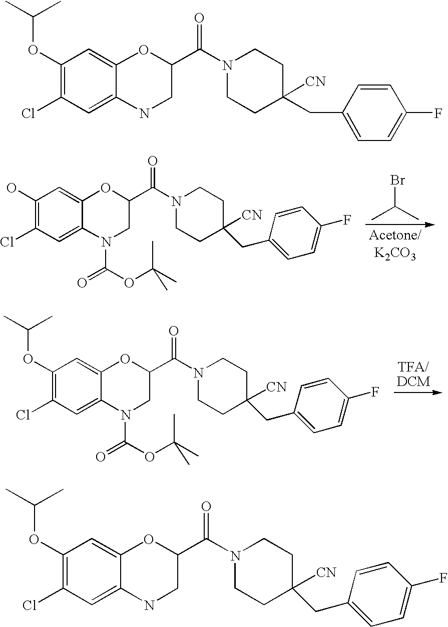

Step 1: 6-Chloro-2-[4-cyano-4-(4-fluoro-benzyl)-piperidine-1-carbonyl]-7-isopropoxy-2,3-dihydro-benzo[1,4]oxazine-4-carboxylic acid tert-butyl ester

To a solution of 6-Chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester (150 mg, 0.28 mmol) in acetone was added K₂CO₃ (78 mg, 0.31 mmol) and isopropyl bromide (0.03 ml, 0.57 mmol) at 0° C. and was refluxed overnight. The reaction mixture was concentrated, diluted water, extracted with ethyl acetate. The organic layer was concentrated under reduced pressure and the residue was purified by flash chromatography (silica-gel, 2% MeOH-DCM) to afford 155 mg (96.9%) of 6-Chloro-2-[4-cyano-4-(4-fluoro-benzyl)-piperidine-1-carbonyl]-7-isopropoxy-2,3-dihydro-benzo[1,4]oxazine-4-carboxylic acid tert-butyl ester. LC/MS [M+H]⁺: 572.2.

Step 2: 1-(6-Chloro-7-isopropoxy-3,4-dihydro-2H-benzo[1,4]oxazine-2-carbonyl)-4-(4-fluoro-benzyl)-piperidine-4-carbonitrile

Trifluoroacetic acid (0.1 ml, 1.33 mmol) was added to 6-Chloro-2-[4-cyano-4-(4-fluoro-benzyl)-piperidine-1-carbonyl]-7-isopropoxy-2,3-dihydro-benzo[1,4]oxazine-4-carboxylic acid tert-butyl ester (155 mg, 0.27 mmol) in DCM at 5-10° C. and the reaction mixture was stirred for 2-4 h at room temperature. The solution was then concentrated and neutralised with 1 (N) NaOH solution. The product was extracted with ethyl acetate and the organic layer was washed with water and brine solution. The organics were concentrated under reduced pressure to afford 100 mg (78.5%) of 1-(6-Chloro-7-isopropoxy-3,4-dihydro-2H-benzo[1,4]oxazine-2-carbonyl)-4-(4-fluoro-benzyl)-piperidine-4-carbonitrile. LC/MS [M+H]⁺: 471.8. ¹H-NMR (400 MHz, DMSO-d₆) δ(ppm): 7.34 (bs, 2H), 7.19 (t, 3H), 6.60 (d, 2H), 5.56 (s, 1H), 4.96 (bs, 1H), 4.33 (m, 2H), 4.05 (m, 1H), 3.21 (m, 2H), 2.94 (s, 2H), 2.70 (s, 1H), 1.80 (m, 3H), 1.50 (m, 1H), 1.22 (s, 6H), 0.84 (m, 1H). HPLC purity: 97.5%.

### Example 61

### 1-(4-Acetyl-6-chloro-7-dimethylamino-3,4-dihydro-2H-benzo[1,4]oxazine-2-carbonyl)-4-(4-fluoro-benzyl)-piperidine-4-carbonitrile

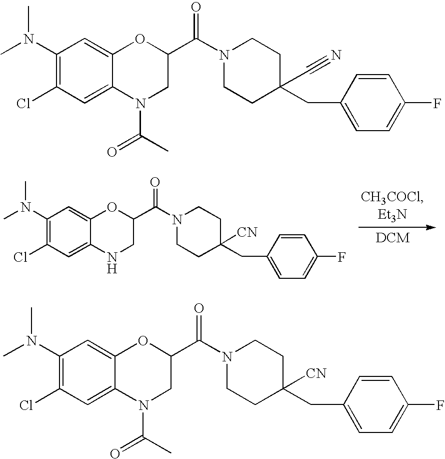

1-(6-Chloro-7-dimethylamino-3,4-dihydro-2H-benzo[1,4]oxazine-2-carbonyl)-4-(4-fluoro-benzyl)-piperidine-4-carbonitrile was prepared as described in example 51.

To a solution of 1-(6-Chloro-7-dimethylamino-3,4-dihydro-2H-benzo[1,4]oxazine-2-carbonyl)-4-(4-fluoro-benzyl)-piperidine-4-carbonitrile (70 mg, 0.15 mmol) in DCM was added triethyl amine (0.02 ml, 0.18 mmol) and acetyl chloride (0.01 ml, 0.16 mmol) at cold condition. The mixture was stirred at room temperature for 1 hr, concentrated, diluted with water and extracted with dichloromethane. The organic layer was washed with sodium bicarbonate solution followed by water, brine and dried over Na₂SO₄. Concentration of organic layer under reduced pressure followed by washing with hexane afforded 50 mg (66.7%) of 1-(4-Acetyl-6-chloro-7-dimethylamino-3,4-dihydro-2H-benzo[1,4]oxazine-2-carbonyl)-4-(4-fluoro-benzyl)-piperidine-4-carbonitrile. LC/MS [M+H]⁺: 499.2. ¹H-NMR (400 MHz, DMSO-d₆) δ(ppm): 7.33 (m, 2H), 7.19 (m, 2H), 6.67 (m, 1H), 4.33 (m, 1H), 4.02 (m, 1H), 4.02 (m, 1H), 2.93 (m, 3H), 2.68 (s, 6H), 2.32 (bs, 3H), 2.00 (s, 1H), 1.82 (m, 3H), 1.53 (m, 2H), 1.23 (s, 1H), 1.19 (m, 1H), 0.85 (m, 1H). HPLC purity: 92.2%.

### Example 62

### 2-{6-Chloro-2-[4-cyano-4-(4-fluoro-benzyl)-piperidine-1-carbonyl]-7-dimethylamino-2,3-dihydro-benzo[1,4]oxazin-4-yl}-acetamide

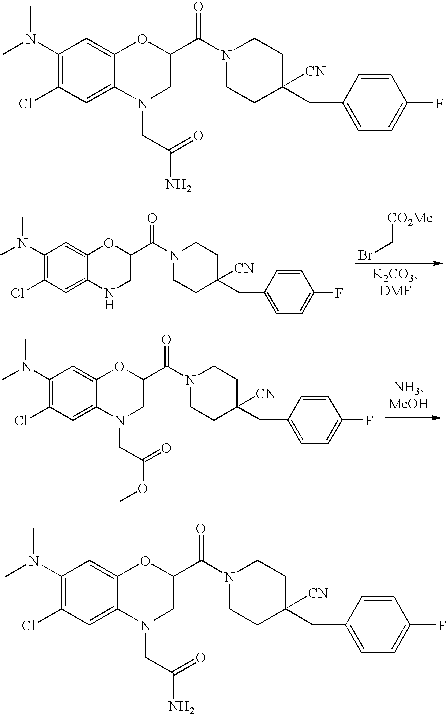

1-(6-Chloro-7-dimethylamino-3,4-dihydro-2H-benzo[1,4]oxazine-2-carbonyl)-4-(4-fluoro-benzyl)-piperidine-4-carbonitrile was prepared as described in example 51.
Step-1: {6-Chloro-2-[4-cyano-4-(4-fluoro-benzyl)-piperidine-1-carbonyl]-7-dimethylamino-2,3-dihydro-benzo[1,4]oxazin-4-yl}-acetic acid methyl ester

To a solution of 1-(6-Chloro-7-dimethylamino-3,4-dihydro-2H-benzo[1,4]oxazine-2-carbonyl)-4-(4-fluoro-benzyl)-piperidine-4-carbonitrile (70 mg, 0.150 mmol) in DMF was added K₂CO₃ (41.5 mg, 0.30 mmol) and methyl bromoacetate (0.014 ml, 0.164 mmole) at 0° C. The Reaction mixture was stirred at room temperature for overnight. The reaction mixture was diluted with cold water and extracted with ethyl acetate. The organic layer was concentrated under reduced pressure to afford 60 mg (75.3%) of (6-Chloro-2-[4-cyano-4-(4-fluoro-benzyl)-piperidine-1-carbonyl]-7-dimethylamino-2,3-dihydro-benzo[1,4]oxazin-4-yl}-acetic acid methyl ester. LC/MS [M+H]⁺: 529.2.

Step-2: 2-{6-Chloro-2-[4-cyano-4-(4-fluoro-benzyl)-piperidine-1-carbonyl]-7-dimethylamino-2,3-dihydro-benzo[1,4]oxazin-4-yl}-acetamide

To a solution of {6-Chloro-2-[4-cyano-4-(4-fluoro-benzyl)-piperidine-1-carbonyl]-7-dimethylamino-2,3-dihydro-benzo[1,4]oxazin-4-yl}-acetic acid methyl ester (60 mg, 0.11 mmol) in methanol was saturated with ammonia gas during 15 minutes at 0° C. The resulting mixture was heated at 60° C. for 12 hrs. The ammonia solution was concentrated under reduced pressure. Purification of the crude mass by column chromatography in 100-200 silica mesh (2%-MeOH-DCM) afforded 40 mg (68.7%) of 2-{6-Chloro-2-[4-cyano-4-(4-fluoro-benzyp-piperidine-1-carbonyl]-7-dimethylamino-2,3-dihydrobenzo[1,4]oxazin-4-yl}-acetamide. LC/MS [M+H]⁺: 514.3. ¹H-NMR (400 MHz, DMSO-d₆) δ(ppm): 7.54 (d, 1H), 7.34 (d, 2H), 7.19 (t, 3H), 6.64 (m, 1H), 6.46 (s, 1H), 5.21 (s, 1H), 4.37 (d, 1H), 4.13 (m, 1H), 3.77 (m, 2H), 3.47 (bs, 2H), 2.72 (m, 1H), 2.59 (s, 6H), 2.49 (s, 2H), 1.82 (m, 4H), 1.54 (m, 1H). HPLC purity: 95.8%.

### Example 63

### 1-(6-Chloro-7-dimethylamino-4-methanesulfonyl-3,4-dihydro-2H-benzo[1,4]oxazine-2-carbonyl)-4-(4-fluoro-benzyl)-piperidine-4-carbonitrile

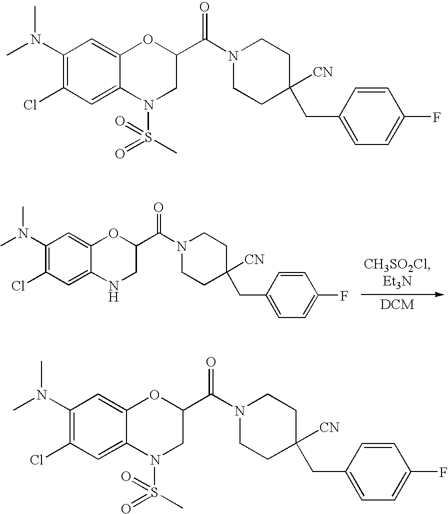

1-(6-Chloro-7-dimethylamino-3,4-dihydro-2H-benzo[1,4]oxazine-2-carbonyl)-4-(4-fluoro-benzyl)-piperidine-4-carbonitrile was prepared as described in example 51.

To a solution of 1-(6-Chloro-7-dimethylamino-3,4-dihydro-2H-benzo[1,4]oxazine-2-carbonyl)-4-(4-fluoro-benzyl)-piperidine-4-carbonitrile (50 mg,0.10 mmol) in DCM was added triethylamine (0.05 ml, 0.36 m.mol) and methane sulfonyl chloride (0.009 ml, 0.12 mmol) in cold condition. The reaction mixture was stirred at room temperature for 1 hr. The reaction mixture was concentrated, diluted with water and extracted with dichloromethane. The organic layer was washed with bicarbonate solution, water, brine and dried over Na₂SO₄. Concentration of organic layer under reduced pressure followed by washing with hexane afforded 35 mg (59.7%) 1-(6-Chloro-7-dimethylamino-4-methanesulfonyl-3,4-dihydro-2H-benzo[1,4]oxazine-2-carbonyl)-4-(4-fluoro-benzyl)-piperidine-4-carbonitrile. LC/MS [M+H]⁺: 535.2. ¹H-NMR (400 MHz, DMSO-d₆) δ(ppm): 7.56 (bs, 1H), 7.36 (m, 2H), 5.19 (m, 1H), 4.42 (m, 1H), 3.58 (m, 1H), 3.35 (m, 1H), 3.25 (d, 3H), 3.17 (m, 1H), 3.03 (m, 1H), 2.92 (bs, 1H), 2.77 (s, 6H), 2.33 (s, 3H), 2.08 (s, 1H), 1.80 (m, 3H). HPLC purity: 93.5%

### Example 64

### 2-{6-Chloro-2-[4-cyano-4-(4-fluoro-benzyl)-piperidine-1-carbonyl]-7-isopropoxy-2,3-dihydro-benzo[1,4]oxazin-4-yl}-acetamide

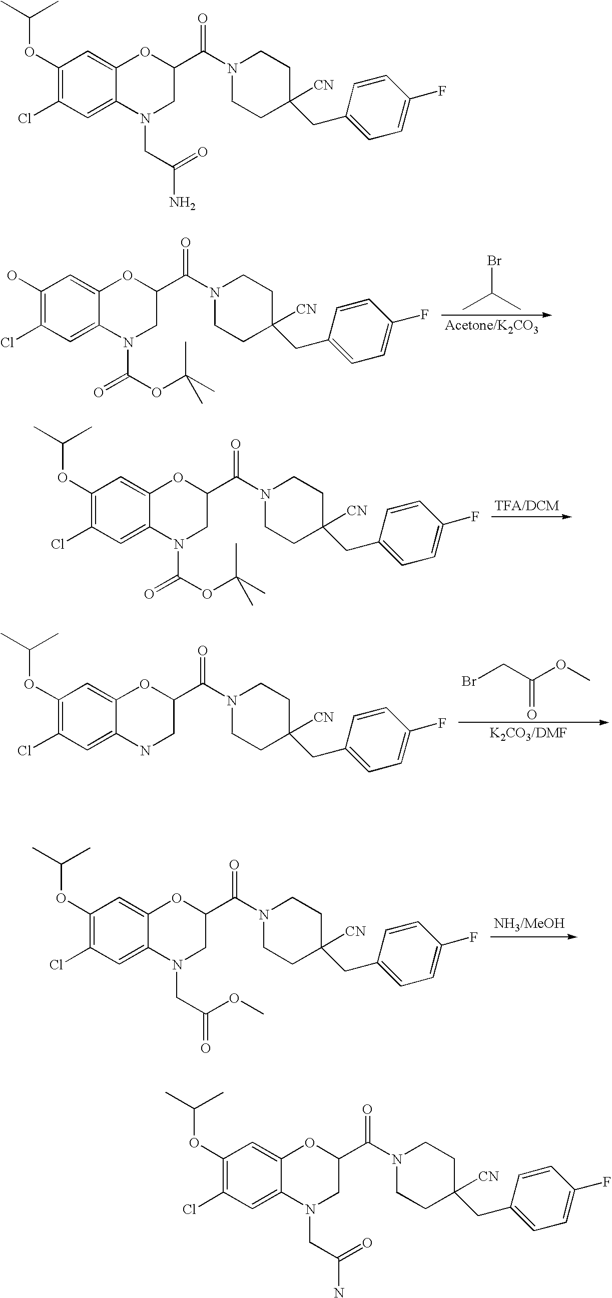

Step 1: 6-Chloro-2-[4-cyano-4-(4-fluoro-benzyl)-piperidine-1-carbonyl]-7-isopropoxy-2,3-dihydro-benzo[1,4]oxazine-4-carboxylic acid tert-butyl ester

To a solution of 6-Chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester (150 mg, 0.28 mmol in acetone was added K₂CO₃ (78 mg, 0.31 mmol) and isopropyl bromide (0.03 ml, 0.57 mmol) at 0° C. and was refluxed overnight. The reaction mixture was concentrated, diluted water, extracted with ethyl acetate. The organic layer was concentrated under reduced pressure and the residue was purified by flash chromatography (silica-gel, 2% MeOH-DCM) to afford 155 mg (96.9%)of 6-Chloro-2-[4-cyano-4-(4-fluoro-benzyl)-piperidine-1-carbonyl]-7-isopropoxy-2,3-dihydro-benzo[1,4]oxazine-4-carboxylic acid tert-butyl ester. LC/MS [M+H]⁺: 572.2.

Step 2: 1-(6-Chloro-7-isopropoxy-3,4-dihydro-2H-benzo[1,4]oxazine-2-carbonyl)-4-(4-fluoro-benzyl)-piperidine-4-carbonitrile

Trifluoroacetic acid (0.1 ml, 1.33 mmol) was added to 6-Chloro-2-[4-cyano-4-(4-fluoro-benzyl)-piperidine-1-carbonyl]-7-isopropoxy-2,3-dihydro-benzo[1,4]oxazine-4-carboxylic acid tert-butyl ester (155 mg, 0.27 mmol) in DCM at 5-10° C. and the reaction mixture was stirred for 2-4 h at room temperature. The solution was then concentrated and neutralised with 1 (N) NaOH solution. The product was extracted with ethyl acetate and the organic layer was washed with water and brine solution. The organics were concentrated under reduced pressure to afford 100 mg (78.5%) of 1-(6-Chloro-7-isopropoxy-3,4-dihydro-2H-benzo[1,4]oxazine-2-carbonyl)-4-(4-fluoro-benzyl)-piperidine-4-carbonitrile. LC/MS [M+H]⁺: 471.8.

Step 3: (6-Chloro-2-[4-cyano-4-(4-fluoro-benzyl)-piperidine-1-carbonyl]-7-isopropoxy-2,3-dihydro-benzo[1,4]oxazin-4-yl}-acetic acid methyl ester

To a solution of 1-(6-Chloro-7-isopropoxy-3,4-dihydro-2H-benzo[1,4]oxazine-2-carbonyl)-4-(4-fluoro-benzyl)-piperidine-4-carbonitrile (70 mg, 0.15 mmol) in DMF was added methyl bromoacetate (0.12 ml,1.31 mmol) and stirred the reaction mixture for overnight. The reaction mixture was heated for 2 hour. The reaction mixture was diluted with ice-water and extracted with ethyl acetate. The organic layer was concentrated under reduced pressure and the residue was purified by flash chromatography (silica-gel, 20% ethyl acetate-hexane) to afford 70 mg (85.7%) {6-Chloro-2-[4-cyano-4-(4-fluoro-benzyl)-piperidine-1-carbonyl]-7-isopropoxy-2,3-dihydro-benzo[1,4]oxazin-4-yl}-acetic acid methyl ester. LC/MS [M+H]⁺:544.0.

Step 4: 2-{6-Chloro-2-[4-cyano-4-(4-fluoro-benzyl)-piperidine-1-carbonyl]-7-isopropoxy-2,3- dihydro-benzo[1,4]oxazin-4-yl}-acetamide

A solution of {6-Chloro-2-[4-cyano-4-(4-fluoro-benzyl)-piperidine-1-carbonyl]-7-isopropoxy-2,3-dihydro-benzo[1,4]oxazin-4-yl}-acetic acid methyl ester (60 mg, 0.11 mmol) in methanol was saturated with ammonia by purging with ammonia gas at atmospheric pressure at −5° C. The resulting mixture was heated overnight at 60° C. in a sealed tube. The mixture was then concentrated and washed with hexane to afford 18 mg (30.9%) 2-{6-Chloro-2-[4-cyano-4-(4-fluoro-benzyl)-piperidine-1-carbonyl]-7-isopropoxy-2,3-dihydro-benzo[1,4]oxazin-4-yl}-acetamide. LC/MS [M+H]⁺: 529.2. ¹H-NMR (400 MHz, DMSO-d₆) δ(ppm): 7.36 (m,2H), 7.24 (m,1H), 7.15 (m,2H), 6.99 (m,1H), 6.55 (m,2H), 5.40 (bs, 1H) 4.38 (m, 1H), 4.26 (m, 1H), 3.81 (s, 1H), 3.48 (m, 2H), 2.96 (s, 2H), 1.88 (m, 2H), 1.66 (m, 2H), 1.54 (m, 1H), 1.36 (m, 2H), 1.24 (s, 6H), 0.87(m,1H). HPLC purity: 93.1%

### Example 65

### 2-[6-Chloro-2-[4-cyano-4-(4-fluoro-benzyl)-piperidine-1-carbonyl]-7-ethoxy-2,3-dihydro-benzo[1,4]oxazin-4-yl}-acetamide

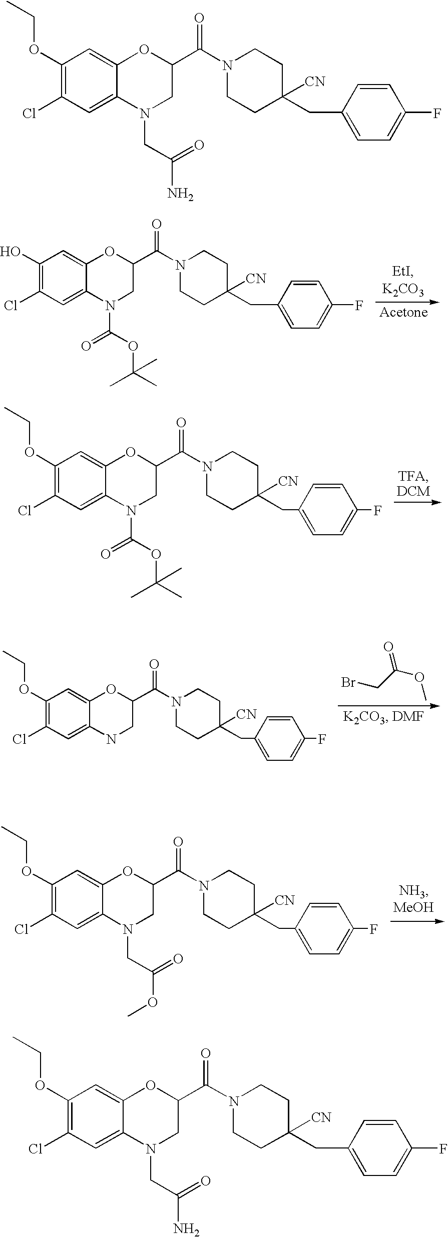

6-Chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester was prepared by a process as described in example 37.
Step-1: 6-Chloro-2-[4-cyano-4-(4-fluoro-benzyl)-piperidine-1-carbonyl]-7-ethoxy-2,3-dihydro-benzo[1,4]oxazine-4-carboxylic acid tert-butyl ester

To a solution of 6-Chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester (500 mg, 0.9433 mmol) in acetone was added K₂CO₃ (260.7 mg, 1.88 mmol) and ethyl Iodide (0.22 ml, 2.83 mmol) in cold condition. The reaction mixture was allowed to reflux for 5 hrs. Then reaction mixture was concentrated and the crude was diluted with water, extracted with ethyl acetate. The organic layer was washed with water and brine and dried over Na₂SO₄. The organics was concentrated under reduced pressure and purified over column chromatography (Si-gel, 20% ethyl acetate-hexane) to afford 430 mg (81.7%) of 6-Chloro-2-[4-cyano-4-(4-fluoro-benzyl)-piperidine-1-carbonyl]-7-ethoxy-2,3-dihydro-benzo[1,4]oxazine-4-carboxylic acid tert-butyl ester. LC/MS [M+H]⁺: 558.0

Step-2: 1-(6-Chloro-7-ethoxy-3,4-dihydro-2H-benzo[1,4]oxazine-2-carbonyl)-4-(4-fluoro-benzyl)-piperidine-4-carbonitrile

To a solution of 6-Chloro-2-[4-cyano-4-(4-fluoro-benzyl)-piperidine-1-carbonyl]-7-ethoxy-2,3-dihydro-benzo[1,4]oxazine-4-carboxylic acid tert-butyl ester (430 mg,0.77 mmol) in DCM was added trifluoroacetic acid (0.4 ml,6.16 mmol) drop wise in cold condition. The reaction mixture was allowed to stir at room temperature for one and half an hour. The reaction mixture was concentrated, diluted with water, basified with 2N NaOH solution and extracted with ethyl acetate. The organic layer was washed with water, brine and dried over Na₂SO₄. Concentration of the organic extract under vacuum followed by washing with hexane afforded 300 mg (85.2%) of compound 1-(6-Chloro-7-ethoxy-3,4-dihydro-2H-benzo[1,4]oxazine-2-carbonyl)-4-(4-fluoro-benzyl)-piperidine-4-carbonitrile. LC/MS [M+H]⁺: 458.3.

Step-3: (6-Chloro-2-[4-cyano-4-(4-fluoro-benzyl)-piperidine-1-carbonyl]-7-ethoxy-2,3-dihydro-benzo[1,4]oxazin-4-yl}-acetic acid methyl ester

To a solution of 1-(6-Chloro-7-ethoxy-3,4-dihydro-2H-benzo[1,4]oxazine-2-carbonyl)-4-(4-fluoro-benzyl)-piperidine-4-carbonitrile (100 mg, 0.22 mmol) in DMF was added K₂CO₃ (61.4 mg, 0.43 mmol) and methyl bromoacetate (0.02 ml, 0.24 mmol) at 0° C. The reaction mixture was stirred for overnight at room temperature. The reaction mixture was diluted with water and extracted with ethyl acetate. The organic layer was concentrated under reduce pressure to afford 90 mg (78.2%) of {6-Chloro-2-[4-cyano-4-(4-fluoro-benzyl)-piperidine-1-carbonyl]-7-ethoxy-2,3-dihydro-benzo[1,4]oxazin-4-yl}-acetic acid methyl ester. LC/MS [M+H]⁺: 530.3

Step-4: 2-{6-Chloro-2-[4-cyano-4-(4-fluoro-benzyl)-piperidine-1-carbonyl]-7-ethoxy-2,3-dihydro-benzo[1,4]oxazin-4-yl}-acetamide

To a solution of {6-Chloro-2-[4-cyano-4-(4-fluoro-benzyl)-piperidine-1-carbonyl]-7-ethoxy-2,3-dihydro-benzo[1,4]oxazin-4-yl}-acetic acid methyl ester (90 mg, 0.16 mmol) in methanol, was saturated with ammonia gas during 15 minutes at 0° C. The resulting mixture was heated at 60° C. for 12 hrs in a sealed tube. Then reaction mass was concentrated under reduced pressure and purified over Si-gel column (2-2.5% MeOH-DCM) to afford 60 mg (68.6%) of 2-{6-Chloro-2-[4-cyano-4-(4-fluoro-benzyp-piperidine-1-carbonyl]-7-ethoxy-2,3-dihydro-benzo[1,4]oxazin-4-yl]-acetamide. LC/MS [M+H]⁺: 515.0. ¹H-NMR (400 MHz, DMSO-d₆) δ(ppm): 7.50 (m, 1H), 7.35 (bs, 2H), 7.19 (t, 3H), 6.62 (m, 1H), 6.49 (bs, 1H), 5.22 (s, 1H), 4.20 (m, 1H), 4.10 (m, 1H), 3.96 (m, 2H), 3.75 (m, 2H), 3.45 (s, 2H), 2.94 (bs, 2H), 2.84 (m, 1H). HPLC purity: 96.6%.

### Example 66

### 1-(6-Chloro-7-ethoxy-4-methanesulfonyl-3,4-dihydro-2H-benzo[1,4]oxazine-2-carbonyl)-4-(4-fluoro-benzyl)-piperidine-4-carbonitrile

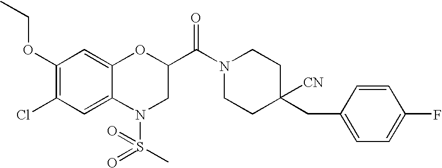

1-(6-Chloro-7-ethoxy-4-methanesulfonyl-3,4-dihydro-2H-benzo[1,4]oxazine-2-carbonyl)-4-(4-fluoro-benzyl)-piperidine-4-carbonitrile was prepared from 1-(6-Chloro-7-ethoxy-3,4-dihydro-2H-benzo[1,4]oxazine-2-carbonyl)-4-(4-fluoro-benzyl)-piperidine-4-carbonitrile by a similar process as described in example 46. LC/MS [M+H]⁺: 536.3. ‘H-NMR (400 MHz, DMSO-d₆) δ(ppm): 7.56-6.48 (m, 6 Ar H), 4.65 (br s, 1H), 4.11-3.44 (m, 4H), 3.06 (s, 3H0, 2.93-2.87 (m, 3H), 1.96 (m, 2H), 1.64-1.42 (m, 5H). HPLC purity: 98.3%.

### Example 67

### 1-(6-Chloro-7-pyrrolidin-1-yl-3,4-dihydro-2H-benzo[1,4]oxazine-2-carbonyl)-4-(4-fluoro-benzyl)-piperidine-4-carbonitrile

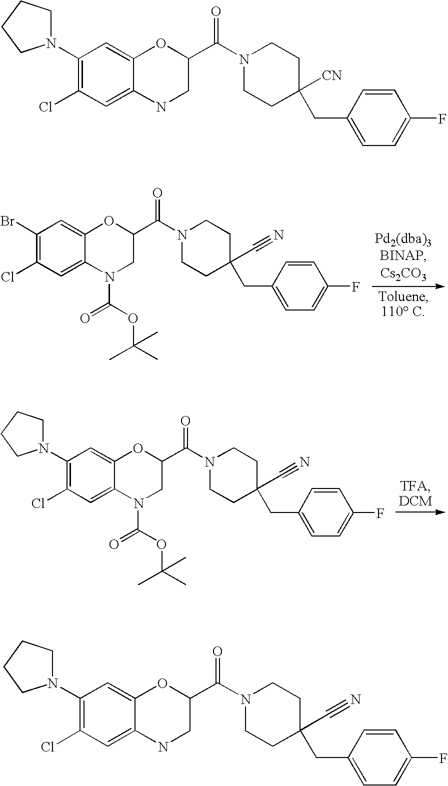

1-(7-Bromo-6-chloro-3,4-dihydro-2H-benzo[1,4]oxazine-2-carbonyl)-4-(4-fluoro-benzyl)-piperidine-4-carbonitrile was prepared by a process described in example 57.
Step-1: 6-Chloro-2-[4-cyano-4-(4-fluoro-benzyl)-piperidine-1-carbonyl]-7-pyrrolidin-1-yl-2,3-dihydro-benzo[1,4]oxazine-4-carboxylic acid tert-butyl ester

To a solution of 1-(7-Bromo-6-chloro-3,4-dihydro-2H-benzo[1,4]oxazine-2-carbonyl)-4-(4-fluoro-benzyl)-piperidine-4-carbonitrile (150 mg, 0.25 mmol) in toluene was added pyrolidine (18 mg, 0.25 mmol) and cesium carbonate (248 mg, 0.75 mmol). The solution was degassed with argon during 1 hour. To the previous degassed solution was added Pd₂(dba)₃ (15 mg, 0.02 mmol) followed by BINAP (32 mg, 0.04 mmol) and was heated at 100 C overnight. The reaction mixture was passed through a celite bed was diluted with water. The organics was extracted with ethyl acetate. The organic layer was concentrated and purified over Si-gel (15% EtOAc-Hex)) to afford 80 mg (54.4%) of 6-Chloro-2-[4-cyano-4-(4-fluoro-benzyl)-piperidine-1-carbonyl]-7-pyrrolidin-1-yl-2,3-dihydro-benzo[1,4]oxazine-4-carboxylic acid tert-butyl ester. LC/MS [M+H]⁺: 583.4

Step-2: 1-(6-Chloro-7-pyrrolidin-1-yl-3,4-dihydro-2H-benzo[1,4]oxazine-2-carbonyl)-4-(4-fluoro-benzyl)-piperidine-4-carbonitrile

To a solution of 6-Chloro-2-[4-cyano-4-(4-fluoro-benzyl)-piperidine-1-carbonyl]-7-pyrrolidin-1-yl-2,3-dihydro-benzo[1,4]oxazine-4-carboxylic acid tert-butyl ester (80 mg, 0.14 mmol) in dichloromethane was added trifluoroacetic acid (0.3 ml). The reaction mixture was stirred for 3 hours and concentrated. The organics was diluted with water, basified with 10% aqueous sodium hydroxide solution and extracted with ethyl acetate. The organic layer was dried over sodium sulfate and concentrated to afford 40 mg (60.6%) of 1-(6-Chloro-7-pyrrolidin-1-yl-3,4-dihydro-2H-benzo[1,4]oxazine-2-carbonyl)-4-(4-fluoro-benzyl)-piperidine-4-carbonitrile. LC/MS [M+H]⁺: 483.4. ‘H-NMR (400 MHz, DMSO-d₆) δ(ppm): 7.06 (m, 4H), 6.69 (bs, 1H), 6.48 (m, 2H), 4.77 (m, 2H), 4.64 (m, 2H), 4.25 (m, 2H), 3.44 (m, 2H), 3.36-2.92 (m, 11H), 2.85 (s, 2H), HPLC purity: 94.1%.

### Example 68

### 1-(7-Azetidin-1-yl-6-chloro-3,4-dihydro-2H-benzo[1,4]oxazine-2-carbonyl)-4-(4-fluoro-benzyl)-piperidine-4-carbonitrile

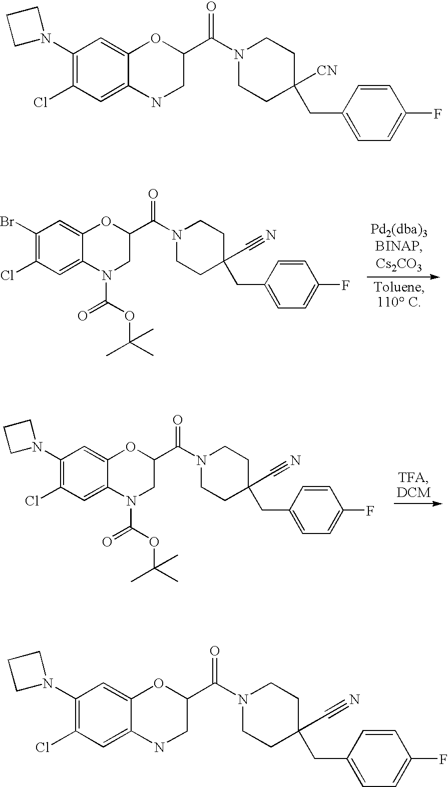

1-(7-Bromo-6-chloro-3,4-dihydro-2H-benzo[1,4]oxazine-2-carbonyl)-4-(4-fluoro-benzyl)-piperidine-4-carbonitrile was prepared by a process described in example 57.

1-(7-Azetidin-1-yl-6-chloro-3,4-dihydro-2H-benzo[1,4]oxazine-2-carbonyl)-4-(4-fluoro-benzyl)-piperidine-4-carbonitrile was prepared by a similar process as described in CF00416. LC/MS [M+H]⁺: 469.4. ¹H-NMR (400 MHz, DMSO-d₆) δ(ppm): 7.46 (m, 2H), 7.21 (m, 2H), 6.57 (s, 1H), 6.09 (m, 1H), 5.43 (s, 1H), 4.91 (bs, 1H), 4.07 (bs, 1H), 4.02 (bs, 1H), 3.76 (s, 4H), 3.18 (m, 2H), 2.95 (bs, 2H), 2.69 (bs, 1H), 2.14 (bs, 2H), 1.79 (m, 4H), 1.69 (m, 1H). HPLC purity: 83.91%.

### Example 69

### 1-(6-Chloro-7-difluoromethoxy-3,4-dihydro-2H-benzo[1,4]oxazine-2-carbonyl)-4-(4-fluoro-benzyl)-piperidine-4-carbonitrile

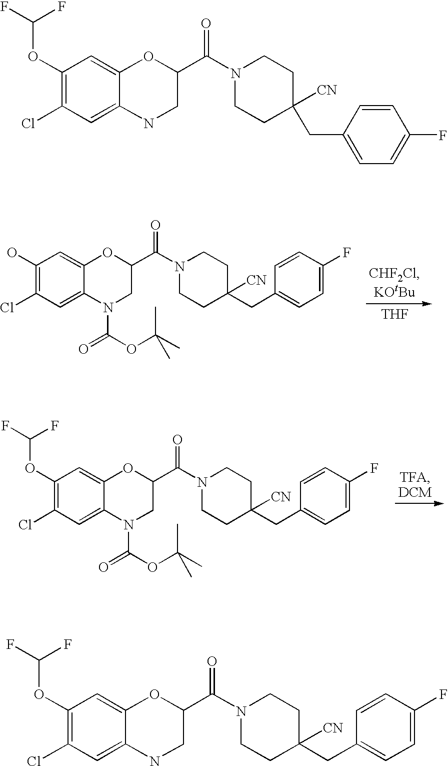

6-Chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester was prepared by a process as described in example 37.
Step-1: 6-Chloro-2-[4-cyano-4-(4-fluoro-benzyl)-piperidine-1-carbonyl]-7-difluoromethoxy-2,3-dihydro-benzo[1,4]oxazine-4-carboxylic acid tert-butyl ester

To a solution of 6-Chloro-2-[4-cyano-4-(4-fluoro-benzyl)-piperidine-1-carbonyl]-7-hydroxy-2,3-dihydro-benzo[1,4]oxazine-4-carboxylic acid tert-butyl ester (200 mg, 0.38 mmol) in THF was added KOtBu (47 mg, 0.43 mmol) at 0° C. and stirred the reaction mixture for one hour. The reaction mixture was cooled to −78° C. and condensed CHF2Cl until its volume became double to that initial. The reaction mixture was stirred at room temperature with Dewar condenser at room temperature for 5 hours. The reaction mixture was concentrated, diluted with ice-water, extracted with ethyl acetate. The organic layer was concentrated under reduced pressure and the residue was purified by flash chromatography (silica-gel, 15% ethyl acetate-hexane) to afford 80 mg (36.8%) of 6-Chloro-2-[4-cyano-4-(4-fluoro-benzyl)-piperidine-1-carbonyl]-7-difluoromethoxy-2,3-dihydro-benzo[1,4]oxazine-4-carboxylic acid tert-butyl ester. LC/MS [M+H]⁺: 580.4. ¹H-NMR (400 MHz, DMSO-d₆) δ(ppm): 7.77 (m, 1H) 7.33 (m, 2H), 7.22 (m, 3H), 6.96 (d, 1H), 4.34 (d, 1H), 4.23 (d, 1H), 3.39 (d, 1H), 3.17 (m, 1H), 2.95 (m, 2H), 2.66 (m, 1H), 1.85 (m, 3H) 1.79 (m, 1H), 1.53 (m, 1H), 1.43 (s, 9H), 1.24 (m, 1H).

### Step-2

### 1-(6-Chloro-7-difluoromethoxy-3,4-dihydro-2H-benzo[1,4]oxazine-2-carbonyl)-4-(4-fluoro- benzyl)-piperidine-4-carbonitrile

Trifluoroacetic acid (0.1 ml, 1.33 mmol) was added of 6-Chloro-2-[4-cyano-4-(4-fluoro-benzyl)-piperidine-1-carbonyl]-7-difluoromethoxy-2,3-dihydro-benzo[1,4]oxazine-4-carboxylic acid tert-butyl ester (80 mg, 0.14 mmol) in DCM at 5-10° C. and the reaction mixture was stirred for 2-4 h at room temperature. The solution was then concentrated and neutralised with 1 (N) NaOH solution. The product was extracted with ethyl acetate and the organic layer was washed with water and brine solution. The organics were concentrated under reduced pressure to afford 53 mg (84.9%) of 1-(6-Chloro-7-difluoromethoxy-3,4-dihydro-2H-1-benzo[1,4]oxazine-2-carbonyl)-4-(4-fluoro-benzyl)-piperidine-4-carbonitrile. LC/MS [M+H]⁺: 480.0. ¹H-NMR (400 MHz, DMSO-d₆) δ(ppm): 7.34 (bs, 2H), 7.19 (t, 2H), 6.81 (m, 1H), 6.69 (s, 1H), 6.15 (s, 11-1), 5.04 (m, 1H), 4.37 (m, 1H), 4.05 (m,1H), 3.40 (m, 1H), 3.31 (m, 1H), 3.15 (m, 1H), 2.94 (s, 2H), 2.72 (m, 1H), 1.98 (s, 3H), 1.72 (m, 1H), 1.24 (m, 1H). HPLC: 94.1%.

### Example 70

### 1-(7-Chloro-6-methoxy-3,4-dihydro-2H-benzo[1,4]oxazine-2-carbonyl)-4-(4-fluoro-benzyl)-piperidine-4-carbonitrile

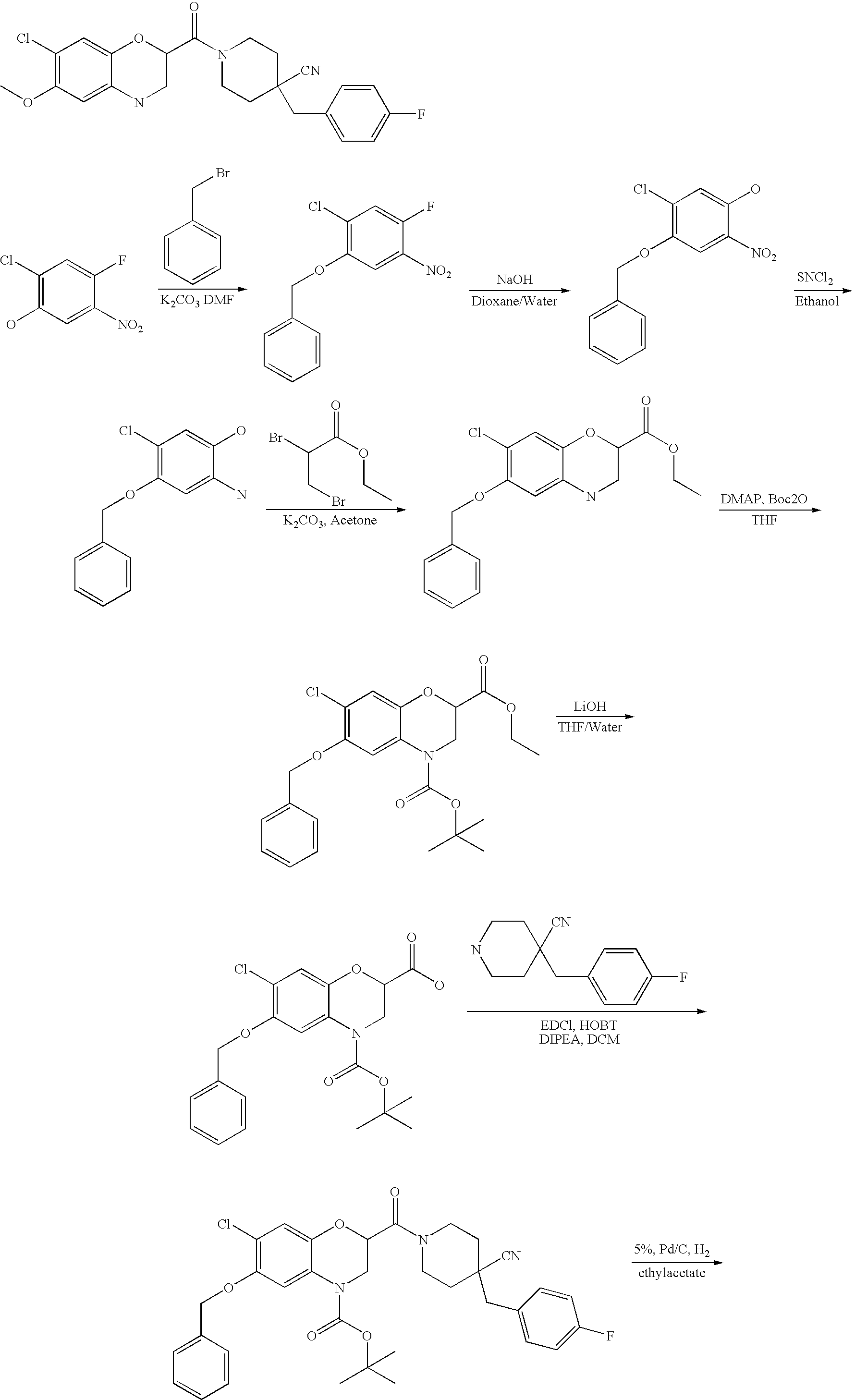

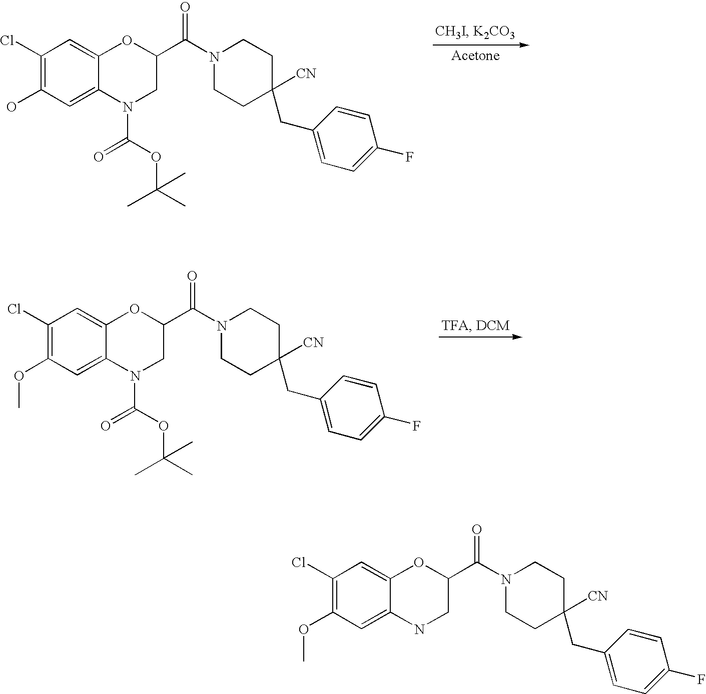

7-Chloro-2-[4-cyano-4-(4-fluoro-benzyl)-piperidine-1-carbonyl]-6-hydroxy-2,3-dihydro-benzo[1,4]oxazine-4-carboxylic acid tert-butyl ester was prepared by a process similar to that described in example 37. LC/MS [M+H]⁺: 530.6.
Step-9: 7-Chloro-2-[4-cyano-4-(4-fluoro-benzyl)-piperidine-1-carbonyl]-6-methoxy-2,3-dihydro-benzo[1,4]oxazine-4-carboxylic acid tent-butyl ester

Anhydrous K₂CO₃ (30.4 mg, 0.22 mmol) and methyl iodide (0.02 ml, 0.34 mmol) were added to 7-Chloro-2-[4-cyano-4-(4-fluoro-benzyl)-piperidine-1-carbonyl]-6-hydroxy-2,3-dihydro-benzo[1,4]oxazine-4-carboxylic acid tert-butyl ester (60 mg, 0.11 mmol) in acetone and the resulting mixture was stirred at 50° C. overnight. The mixture was then diluted with crushed ice and extracted with ethyl acetate. The organic layer was concentrated and purified through column chromatography over Si-gel (20% ethyl acetate-hexane) to afford 10 mg (2%) of 7-Chloro-2-[4-cyano-4-(4-fluoro-benzyl)-piperidine-1-carbonyl]-6-methoxy-2,3-dihydro-benzo[1,4]oxazine-4-carboxylic acid tert-butyl ester. LC/MS [M+H]⁺: 544.6.

Step-10: 1-(7-Chloro-6-methoxy-3,4-dihydro-2H-benzo[1,4]oxazine-2-carbonyl)-4-(4-fluoro-benzyl)-piperidine-4-carbonitrile

To a solution of 7-Chloro-2-[4-cyano-4-(4-fluoro-benzyl)-piperidine-1-carbonyl]-6-methoxy-2,3-dihydro-benzo[1,4]oxazine-4-carboxylic acid tert-butyl ester (10 mg, 0.02 mmol)) in DCM was added trifluoroacetic acid (0.1 ml). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 6 mg (75%) of 1-(7-Chloro-6-methoxy-3,4-dihydro-2H-benzo[1,4]oxazine-2-carbonyl)-4-(4-fluoro-benzyl)-piperidine-4-carbonitrile. LC/MS [M+H]⁺: 444.3. HPLC: 87%.

### Example 71

### 1-(6-Chloro-7-diethylamino-3,4-dihydro-2H-benzo[1,4]oxazine-2-carbonyl)-4-(4-fluoro-benzyl)-piperidine-4-carbonitrile

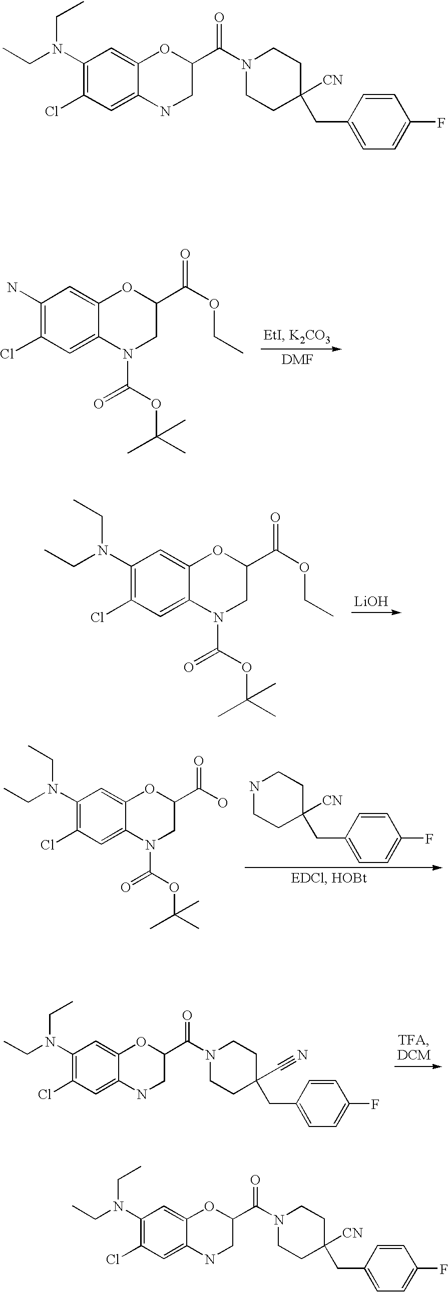

7-Amino-6-chloro-2,3-dihydro-benzo[1,4]oxazine-2,4-dicarboxylic acid 4-tert-butyl ester 2-ethyl ester was prepared by a process as described in example 51.
Step-1: 6-Chloro-7-diethylamino-2,3-dihydro-benzo[1,4]oxazine-2,4-dicarboxylic acid 4-tert-butyl ester 2-ethyl ester

To a solution of 7-Amino-6-chloro-2,3-dihydro-benzo[1,4]oxazine-2,4-dicarboxylic acid 4-tert-butyl ester 2-ethyl ester (500 mg, 1.40 mmol) in DMF was added K₂CO₃ (774.6 mg, 5.60 mmol) and ethyl Iodide (0.56 ml, 7.00 mmol) in cold condition. The reaction mixture was allowed to reflux for 10 hrs. The reaction mass was diluted with water and extracted with ethyl acetate. The organic layer was washed with water, brine and dried over Na₂SO₄. The organics was concentrated under reduced pressure followed by washing with hexane to afford 500 mg (86.5%) of 6-Chloro-7-diethylamino-2,3-dihydro-benzo[1,4]oxazine-2,4-dicarboxylic acid 4-tert-butyl ester 2-ethyl ester. LC/MS [M+H]⁺: 413.0.

Step-2: 6-Chloro-7-diethylamino-2,3-dihydro-benzo[1,4]oxazine-2,4-dicarboxylic acid 4-tert-butyl ester

To a solution of 6-Chloro-7-diethylamino-2,3-dihydro-benzo[1,4]oxazine-2,4-dicarboxylic acid 4-tert-butyl ester 2-ethyl ester (500 mg, 0.35 mmol) in THF-water (3:1) was added LiOH.H₂O (55.8 mg, 1.33 mmol) in cold condition. The mixture was allowed to stir at room temperature for one and half an hour. Then THF was evaporated and the rest mass was diluted with water, neutralized with AcOH and extracted with ethyl acetate. Ethyl acetate extract was washed with water, brine. The organic layer was dried over Na₂SO₄ and concentrated under reduced pressure followed by washing of crude mass with hexane to afford 350 mg (75.2%) of 6-Chloro-7-diethylamino-2,3-dihydro-benzo[1,4]oxazine-2,4-dicarboxylic acid 4-tert-butyl ester. LC/MS [M+H]^{±}: 384.9.

Step-3: 6-Chloro-2-[4-cyano-4-(4-fluoro-benzyl)-piperidine-1-carbonyl]-7-diethylamino-2,3-dihydro-benzo[1,4]oxazine-4-carboxylic acid tert-butyl ester

To a solution of 6-Chloro-7-diethylamino-2,3-dihydro-benzo[1,4]oxazine-2,4-dicarboxylic acid 4-tert-butyl ester (350 mg, 0.91 mmol) in DMF was added EDCI (226.6 mg, 1.18 mmol), HOBT (61.4 mg, 0.45 mmol) and DIPEA (0.5 ml, 2.72 mmol) at 0° C. The resulting mixture was allowed to stir at room temperature for 30 minutes. Then a solution of 4-(4-Fluoro-benzyl)-piperidine-4-carbonitrile in DMF was added to it at 5° C. and the reaction mixture was allowed to stir at room temperature for overnight. The reaction mass was diluted with ice water, stirred for 10 minutes and extracted with ethyl acetate. Ethyl acetate layer was washed with water and brine, then dried over Na₂SO₄ and concentrated under reduced pressure. The crude mass was purified over Si gel column (0.5%MeOH-DCM) to afford 350 mg (65.7%) of compound 6-Chloro-2-[4-cyano-4-(4-fluoro-benzyl)-piperidine-1-carbonyl]-7-diethylamino-2,3-dihydro-benzo[1,4]oxazine-4-carboxylic acid tert-butyl ester. LC/MS [M+H]⁺: 585.3.

Step-4: 1-(6-Chloro-7-diethylamino-3,4-dihydro-2H-benzo[1,4]oxazine-2-carbonyl)-4-(4-fluoro-benzyl)-piperidine-4-carbonitrile

To a solution of 6-Chloro-2-[4-cyano-4-(4-fluoro-benzyl)-piperidine-1-carbonyl]-7-diethylamino-2,3-dihydro-benzo[1,4]oxazine-4-carboxylic acid tert-butyl ester (350 mg,0.59 mmol) in DCM was added trifluoroacetic acid (0.36 ml,4.78 mmol) drop wise in cold condition. The reaction mixture was allowed to stir at room temperature for 3 hours. Then DCM was concentrated. The crude mass was diluted with water, basified with 2N NaOH solution and extracted with ethyl acetate. The organic layer was concentrated under reduced pressure to afford 250 mg (86.2%) of 1-(6-Chloro-7-diethylamino-3,4-dihydro-2H-benzo[1,4]oxazine-2-carbonyl)-4-(4-fluoro-benzyl)-piperidine-4-carbonitrile.

LC/MS [M+H]⁺: 485.2. ¹H-NMR (400 MHz, DMSO-d₆) δ (ppm): 7.73 (s, 2H), 7.19 (t, 2H), 6.59 (d, 2H), 5.85 (s, 1H), 4.97 (s, 1H), 4.38 (d, 1H), 4.09 (m, 1H), 3.15 (m, 2H), 2.94 (s, 2H), 2.89 (s, 4H), 2.71 (d, 1H), 1.98 (m, 4H), 1.72 (bs, 1H), 0.98 (s, 6H). HPLC: 96.3%.

### Example 72

### 1-(6-Chloro-7-morpholin-4-yl-3,4-dihydro-2H-benzo[1,4]oxazine-2-carbonyl)-4-(4-fluoro-benzyl)-piperidine-4-carbonitrile

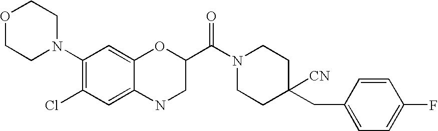

1-(6-Chloro-7-morpholin-4-yl-3,4-dihydro-2H-benzo[1,4]oxazine-2-carbonyl)-4-(4-fluoro-benzyl)-piperidine-4-carbonitrile was prepared by a similar process as described in example 67. LC/MS [M+H]⁺: 498.9. ¹H-NMR (400 MHz, DMSO-d₆) δ (ppm): 7.34 (bs, 2H), 7.19 (m, 2H), 6.64 (m, 1H), 6.54 (m, 1H), 5.81 (s, 1H), 4.96 (m, 1H), 4.41 (m, 1H), 3.69 (m, 1H), 3.36 (bs, 4H), 3.22 (m, 1H), 3.15 (m, 2H), 2.95 (bs, 2H0, 2.80 (m, 4H), 1.98-1.73 (m, 5H). HPLC: 96.4%

### Example 73

### (6-Chloro-7-methoxy-3,4-dihydro-2H-benzo[1,4]oxazin-2-yl)-[4-(4-fluoro-phenoxy)-piperidin-1-yl]-methanone

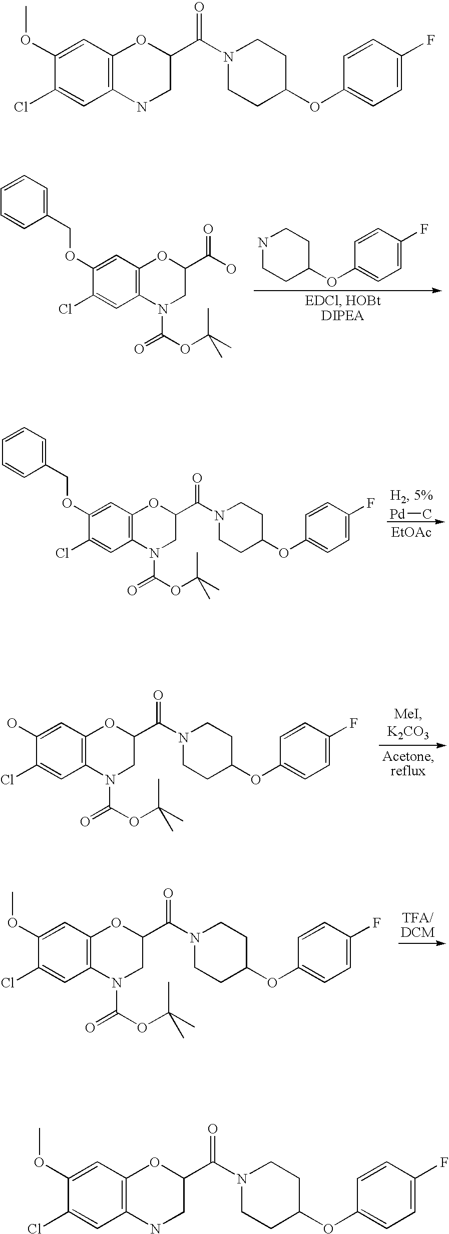

4-(4-Floro-phenoxy)-piperidine was prepared by a process similar to that described in example 64. 7-Benzyloxy-6-chloro-2,3-dihydro-benzo[1,4]oxazine-2,4-dicarboxylic acid 4-tert-butyl ester was prepared by a process similar to that described in example 37 of priority application.
Step-1: 7-Benzyloxy-6-chloro-2-[4-(4-fluoro-phenoxy)-piperidine-1-carbonyl]-2,3-dihydro-benzo[1,4]oxazine-4-carboxylic acid tent-butyl ester

To a solution of 7-Benzyloxy-6-chloro-2,3-dihydro-benzo[1,4]oxazine-2,4-dicarboxylic acid 4-tert-butyl ester (250 mg, 0.59 mmol) in DMF was added 4-(4-Floro-phenoxy)-piperidine (115.2 mg, 0.59 mmol), EDCI (147.6 mg, 0.77 mmol), HOBt (39.2 mg, 0.3 mmol) and DIPEA (0.5 ml, 2.95 mmol) at 5-10° C. The reaction mixture was stirred at room temperature for 15 hours then diluted with water and extracted with ethyl acetate. The ethyl acetate layer was concentrated in vacuo and subjected to column chromatography (Si-gel, 15% EtOAc-hexane) to afford 50 mg (14.2%) of 7-Benzyloxy-6-chloro-2-[4-(4-fluoro-phenoxy)-piperidine-1-carbonyl]-2,3-dihydro-benzo[1,4]oxazine-4-carboxylic acid tert-butyl ester. LC/MS [M+H]⁺: 597.4.

Step-2: 6-Chloro-2-[4-(4-fluoro-phenoxy)-piperidine-1-carbonyl]-7-hydroxy-2,3-dihydro-benzo[1,4]oxazine-4-carboxylic acid tert-butyl ester

7-Benzyloxy-6-chloro-2-[4-(4-fluoro-phenoxy)-piperidine-1-carbonyl]-2,3-dihydro-benzo[1,4]oxazine-4-carboxylic acid tert-butyl ester (140 mg, 0.23 mmol) dissolved in ethyl acetate was hydrogenated with 5% Pd—C (50 mg) for 4 hour. The catalyst was removed by filtration through a celite bed. Concentration of the organics afforded 110 mg (94.3%) of 6-Chloro-2-[4-(4-fluoro-phenoxy)-piperidine-1-carbonyl]-7-hydroxy-2,3-dihydro-benzo[1,4]oxazine-4-carboxylic acid tert-butyl ester. LC/MS [M+H]⁺: 507.1.

Step-3: 6-Chloro-2-[4-(4-fluoro-phenoxy)-piperidine-1-carbonyl]-7-methoxy-2,3-dihydro-benzo[1,4]oxazine-4-carboxylic acid tent-butyl ester

To a solution of 6-Chloro-2-[4-(4-fluoro-phenoxy)-piperidine-1-carbonyl]-7-hydroxy-2,3-dihydro-benzo[1,4]oxazine-4-carboxylic acid tert-butyl ester (110 mg, 0.22 mmol) in acetone was added K₂CO₃ (60 mg, 0.43 mmol) and methyl iodide (0.04 ml, o.65 mmol). The reaction mixture was refluxed overnight and was filtered. The organics was concentrated and purified over Si-gel (20% EtOAc-hexane) to afford 110 mg (95.9%) of 6-Chloro-2-[4-(4-fluoro-phenoxy)-piperidine-1-carbonyl]-7-methoxy-2,3-dihydro-benzo[1,4]oxazine-4-carboxylic acid tert-butyl ester. LC/MS [M+H]⁺: 521.6

Step-4: (6-Chloro-7-methoxy-3,4-dihydro-2H-benzo[1,4]oxazin-2-yl)-[4-(4-fluoro-phenoxy)-piperidin-1-yl]-methanone

Trifluoroacetic acid (0.2 ml, 2.11 mmol) was added of 6-Chloro-2-[4-cyano-4-(4-fluoro-benzyl)-piperidine-1-carbonyl]-7-difluoromethoxy-2,3-dihydro-benzo[1,4]oxazine-4-carboxylic acid tert-butyl ester (110 mg,0.211 mmol) in DCM at 5-10° C. and the reaction mixture was stirred for 2-4 h at room temperature. The solution was then concentrated and neutralised with 1 (N) NaOH solution. The product was extracted with ethyl acetate and the organic layer was washed with water and brine solution. The organics were concentrated under reduced pressure to afford 50 mg (56.5%) of 1-(6-Chloro-7-difluoromethoxy-3,4-dihydro-2H-benzo[1,4]oxazine-2-carbonyl)-4-(4-fluoro-benzyl)-piperidine-4-carbonitrile. LC/MS [M+H]⁺: 421.4. ¹H-NMR (400 MHz, DMSO-d₆) δ (ppm): 7.12 (t, 2H), 7.02 (m, 2H), 6.64 (s, H), 6.58 (bs, H), 5.59 (s, H), 4.96 (s, H), 4.59 (bs, H), 3.82 (m, 2H), 3.47 (s, 3H), 3.33 (m, 2H), 3.21 (m, 2H), 1.98 (m, 3H). HPLC: 93.8%.

### Example 74

### 1-[6-Chloro-4-(2-hydroxy-ethyl)-7-methoxy-3,4-dihydro-2H-benzo[1,4]oxazine-2-carbonyl]-4-(4-fluoro-benzyl)-piperidine-4-carbonitrile

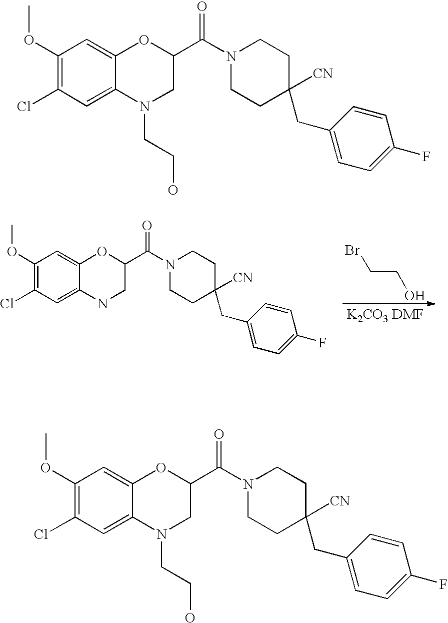

1-(6-Chloro-7-methoxy-3,4-dihydro-2H-benzo[1,4]oxazine-2-carbonyl)-4-(4-fluoro-benzyl)-piperidine-4-carbonitrile was prepared by a process as described in example 27.

To a solution of 1-(6-Chloro-7-methoxy-3,4-dihydro-2H-benzo[1,4]oxazine-2-carbonyl)-4-(4-fluoro-benzyl)-piperidine-4-carbonitrile (100 mg,0.23 mmol) in DMF was added bromoethanol (0.017 ml,0.25 mmol) and K₂CO₃(63.5 mg, 0.46 mmol). The reaction mixture was warmed at 60° C. overnight. The reaction mixture was diluted with water and extracted with ethyl acetate. The organic layer was washed with water and concentrated under reduced pressure to afford 70 mg (62.4%) of 1-[6-Chloro-4-(2-hydroxy-ethyl)-7-methoxy-3,4-dihydro-2H-benzo[1,4]oxazine-2-carbonyl]-4-(4-fluoro-benzyl)-piperidine-4-carbonitrile. LC/MS [M+H]⁺: 488.6. ¹H-NMR (400 MHz, DMSO-d₆) δ (ppm): 7.25-6.48 (m, 6Ar—H), 4.91-4.33 (m, 3H), 3.84-2.8 (m, 14H), 1.89 (m, 2H), 1.41-1.24 (m, 2H). HPLC: 93.4%.

### Example 75

### 1-[6-Chloro-7-dimethylamino-4-(2-hydroxy-ethyl)-3,4-dihydro-2H-benzo[1,4]oxazine-2-carbonyl]-4-(4-fluoro-benzyl)-piperidine-4-carbonitrile

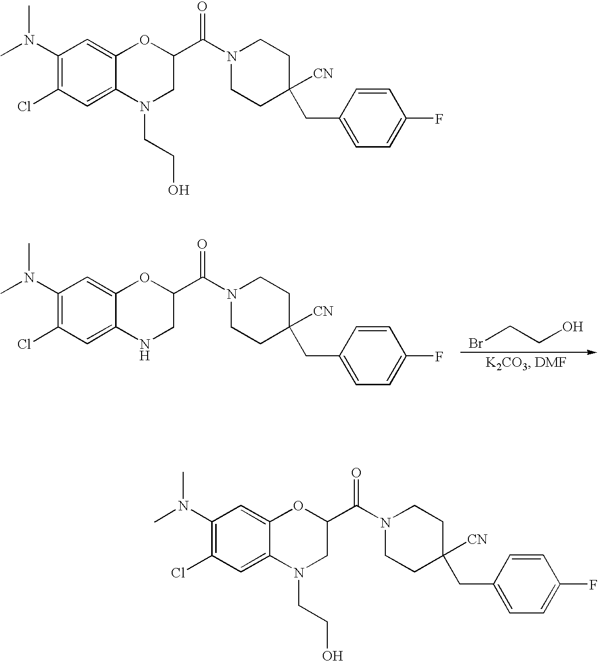

1-(6-Chloro-7-dimethylamino-3,4-dihydro-2H-benzo[1,4]oxazine-2-carbonyl)-4-(4-fluoro-benzyl)-piperidine-4-carbonitrile was prepared as described in example 51.

To a solution of 1-(6-Chloro-7-dimethylamino-3,4-dihydro-2H-benzo[1,4]oxazine-2-carbonyl)-4-(4-fluoro-benzyl)-piperidine-4-carbonitrile (100 mg, 0.21 mmol) in DMF was added K₂CO₃ (60.4 mg, 0.43 mmol) and 2-Bromo ethanol (0.016 ml, 0.24 mmol). The reaction mixture was heated at 55° C. for 36 hrs. The reaction mass was diluted with water and extracted with ethyl acetate. The Organic layer was washed with water, brine and dried over Na₂SO₄. The organics was concentrated under reduced pressure and purified through Si-gel column (50-55% EtOAc-hexane) to afford 40 mg (36.6%) of 1-[6-Chloro-7-dimethylamino-4-(2-hydroxy-ethyl)-3,4-dihydro-2H-benzo[1,4]oxazine-2-carbonyl]-4-(4-fluoro-benzyl)-piperidine-4-carbonitrile. LC/MS [M+H]⁺:501.4. ¹H-NMR (400 MHz, DMSO-d₆) δ (ppm): 7.34 (bs, 2H), 7.19 (t, 2H), 6.72 (s, 1H), 6.62(m, 1H), 4.99 (s, 1H), 4.70 (bs, 1H), 4.40 (m, 1H), 4.10 (m, 1H), 3.55 (bs, 2H), 3.46 (s, 2H), 3.32 (m, 2H), 3.25 (m, 1H), 2.93 (d, 2H), 2.67 (s, 1H), 2.58 (bs, 6H), 1.79(m, 3H), 1.53(m, 1H). HPLC: 95.6%

### Example 76

### 6-Chloro-2-[4-cyano-4-(4-fluoro-benzyl)-piperidine-1-carbonyl]-7-dimethylamino-2,3-dihydro-benzo[1,4]oxazine-4-carbonitrile

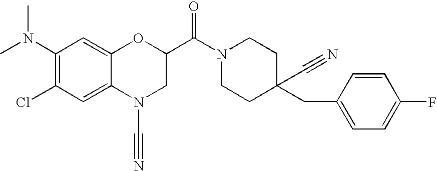

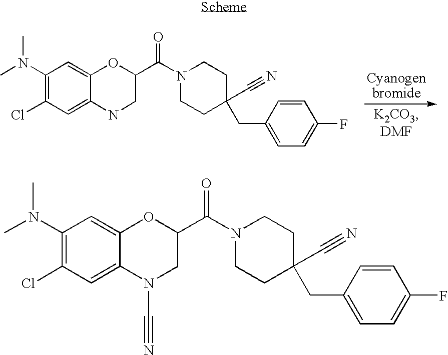

1-(6-Chloro-7-dimethylamino-3,4-dihydro-2H-benzo[1,4]oxazine-2-carbonyl)-4-(4-fluoro-benzyl)-piperidine-4-carbonitrile was prepared as described in example 51.

To a solution of 1-(6-Chloro-7-dimethylamino-3,4-dihydro-2H-benzo[1,4]oxazine-2-carbonyl)-4-(4-fluoro-benzyl)-piperidine-4-carbonitrile (100 mg, 0.22 mmol) in DMF was added K₂CO₃ (60 mg, 0.44 mmol) and cyanogen bromide (28 mg, 0.26 mmol) at cold condition. The reaction mixture was stirred for overnight. The reaction mixture was diluted with water and extracted with ethyl acetate. The organic layer was concentrated under reduced pressure and the residue was purified by flash chromatography (silica-gel, 2% MeOH-DCM) to afford 50 mg (48%) of 6-Chloro-2-[4-cyano-4-(4-fluoro-benzyl)-piperidine-1-carbonyl]-7-dimethylamino-2,3-dihydro-benzo[1,4]oxazine-4-carbonitrile. LC/MS [M+H]⁺:482.4. ¹H-NMR (400 MHz, DMSO-d₆) δ (ppm):7.34(d, 2H, J=5.88), 7.19(t, 2H), 6.98(brs, 1H), 6.78(m, 1H), 4.38(m, 1H), 4.36(m, 1H), 4.16(m, 2H), 3.91(m,2H), 2.94(s, 2H), 2.67(m, 6H), 1.90 to 0.95(5H).

HPLC: 87.7%.

### Example 77

### 1-(6-Chloro-7-dimethylamino-3,4-dihydro-2H-benzo[1,4]oxazine-2-carbonyl)-4-(6-fluoro-pyridin-3-ylmethyl)-piperidine-4-carbonitrile

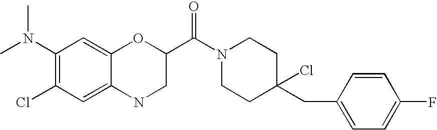

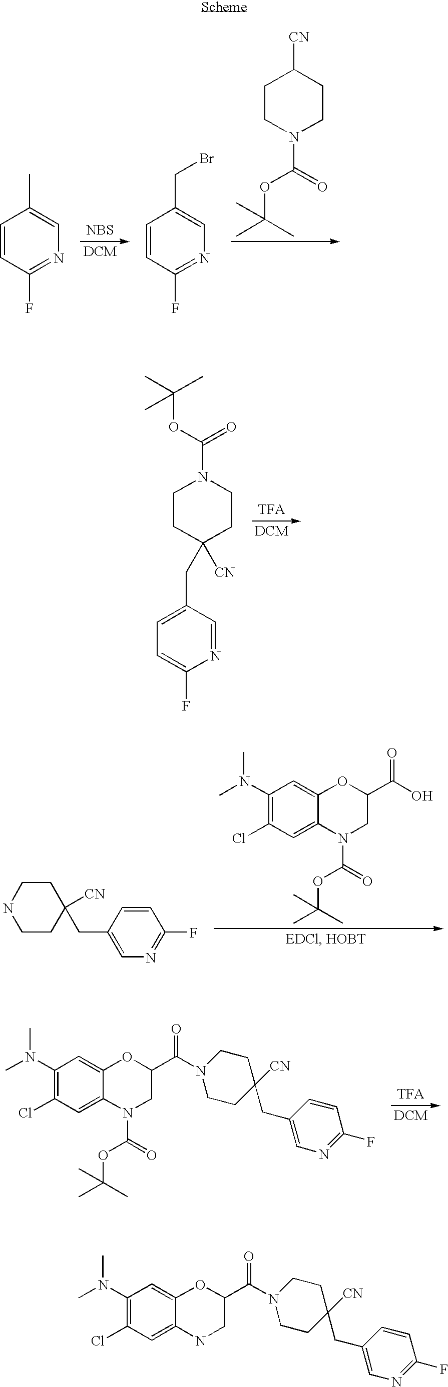

Step-1: 5-Bromomethyl-2-fluoro-pyridine

To a solution of 2-Fluoro-5-methyl-pyridine (500 mg, 4.5 mmol) in 10 ml of CCl₄ NBS (800 mg, 4.5 mmol) and benzoyl peroxide (25 mg) was added in cold condition. The resulting mixture was allowed to stir at room temperature for 30 minutes and then refluxed for one hour.

The reaction mixture was cooled and diluted with water. The organic layer was separated and washed with sodium bicarbonate solution followed by water and brine, then dried over Na₂SO₄ and concentrated under vacuum. The crud mass was purified by column chromatography (Si-gel, 5% ethyl acetate) to afford 350 mg (41%) of 5-Bromomethyl-2-fluoro-pyridine.GCMS (M/z): 189. ¹H-NMR (400 MHz, DMSO-d₆) δ (ppm): 8.34 (s, H), 8.04 (m, H), 7.21 (dd, 1H), 4.76 (s, 2H).

Step-2: 4-Cyano-4-(6-fluoro-pyridin-3-ylmethyl)-piperidine-1-carboxylic acid tert-butyl ester

To a solution of of DIPEA (0.3 ml, 2.21 mmol) in dry THF, was added n-BuLi (2.58 mmol) solution dropwise at 0° C. The mixture was allowed to stir for 1 hr at room temperature. To this mixture a solution of N-boc-4-cyanopipyridine (387 mg, 1.84 mmol) in dry THF was added dropwise at −78° C. The resulting mixture was allowed to stir for two hours at −78° C. to 0° C. Then a solution of 5-Bromomethyl-2-fluoro-pyridine (350 mg, 1.84 mmol) in dry THF was added dropwise at −78° C. and the resulting mixture was allowed to stir at room temperature overnight. The reaction was quenched with saturated NH₄Cl solution in cold condition and stirred at room temperature for 30 minutes. THF layer was separated and the aqueous layer was extracted with ethyl acetate. The combined organic layers (THF and ethyl acetate) was washed with water and brine, then dried over Na₂SO₄ and concentrated under vacuum. The crud mass was purified by column chromatography (Si-gel, 12% ethyl acetate-hexane) to afford 400 mg (68%) of 4-Cyano-4-(6-fluoro-pyridin-3-ylmethyl)-piperidine-1-carboxylic acid tert-butyl ester. ¹H-NMR (400 MHz, DMSO-d₆) δ (ppm):8.16 (s, H), 7.92 (t, 1H, J=8.24 Hz), 7.20 (d, H, J=8.36 Hz), 4.01 (d, 2H, J=13.36 Hz), 2.98 (s, 2H), 2.81(bs, 2H), 1.76 (d, 2H, J=13.28 Hz), 1.56 (m, 2H), 1.39 (s, 9H). LCMS [M+H]⁺:320.2.

Step 3: 4-(6-Fluoro-pyridin-3-ylmethyl)-piperidine-4-carbonitrile

To a solution of 4-Cyano-4-(6-fluoro-pyridin-3-ylmethyl)-piperidine-1-carboxylic acid tert-butyl ester (400 mg, 1.25 mmol) in DCM was added trifluoroacetic acid (0.97 ml, 12.52 mmol) dropwise in cold condition. The mixture was allowed to stir at room temperature for one and half an hour. Then DCM was evaporated and the crude mass was diluted with water, basified with 2N NaOH solution and extracted with ethyl acetate. Ethyl acetate extract was washed with water and brine, then dried over Na₂SO₄. The organic layer was concentrated under vacuum to afforde 230 mg (84%) of 4-(6-Fluoro-pyridin-3-ylmethyl)-piperidine-4-carbonitrile. ¹H-NMR (400 MHz, DMSO-d₆) δ (ppm): 8.14 (s, H), 7.91 (t, H, J=8.2 Hz), 7.19 (d, H, J=8.36 Hz), 2.93 (s, 4H), 2.56 (m, 2H), 1.66 (d, 2H, J=12.6 Hz), 1.48 (m, 2H). LCMS [M+H]⁺: 220.2.

Step-4: 6-Chloro-2-[4-cyano-4-(6-fluoro-pyridin-3-ylmethyl)-piperidine-1-carbonyl]-7-dimethylamino-2,3-dihydro-benzo[1,4]oxazine-4-carboxylic acid tent-butyl ester

To a solution of 6-Chloro-7-dimethylamino-2,3-dihydro-benzo[1,4]oxazine-2,4-dicarboxylic acid 4-tert-butyl ester (114 mg, 0.32 mmol) in DMF was added 4-(6-Fluoro-pyridin-3-ylmethyl)-piperidine-4-carbonitrile (70 mg, 0.32 mmol), EDCI (183 mg, 0.957 mmol), HOBT (22 mg, 0.16 mmol) and DIPEA (0.16 ml, 0.957 mmol) at 5-10° C. and the mixture was stirred at room temperature for 14-16 hours. The reaction mixture was then diluted with water and extracted with ethyl acetate. The organic layer was concentrated under reduced pressure and the residue was purified by flash chromatography (silica-gel, 2% MeOH-DCM) to afford 105 mg (59%) of 6-Chloro-2-[4-cyano-4-(6-fluoro-pyridin-3-ylmethyl)-piperidine-1-carbonyl]-7-dimethylamino-2,3-dihydro-benzo[1,4]oxazine-4-carboxylic acid tert-butyl ester. LC/MS [M+H]⁺: 558.3.

Step-5: 1-(6-Chloro-7-dimethylamino-3,4-dihydro-2H-benzo[1,4]oxazine-2-carbonyl)-4-(6-fluoro-pyridin-3-ylmethyl)-piperidine-4-carbonitrile

Trifluoroacetic acid (0.14 ml, 1.8 mmol) was added to 6-Chloro-2-[4-cyano-4-(6-fluoro-pyridin-3-ylmethyl)-piperidine-1-carbonyl]-7-dimethylamino-2,3-dihydro-benzo[1,4]oxazine-4-carboxylic acid tert-butyl ester (100 mg, 0.179 mmol) in DCM at 5-10 ° C. and the reaction mixture was stirred for 2-4 h at room temperature. The solution was then concentrated and neutralised with 2 (N) NaOH solutions. The product was extracted with ethyl acetate and the organic layer was washed with water and brine solution. The organics were concentrated under reduced pressure to afford 50 mg (61%) of 1-(6-Chloro-7-dimethylamino-3,4-dihydro-2H-benzo[1,4]oxazine-2-carbonyl)-4-(6-fluoro-pyridin-3-ylmethyl)-piperidine-4-carbonitrile. LC/MS [M+H]⁺:458.0.

¹H-NMR (400 MHz, DMSO-d₆) δ (ppm): 8.71(s, 1H), 7.93 (s, 1H), 7.22 (d, 1H, J=2.16), 6.61 (s, 1H), 6.57 (s, 1H), 5.75 (s, 1H), 4.95 (s, 1H), 4.40 (d, 1H, J=11.44), 4.10 (m, 1H), 3.33-1.39 (16H). HPLC: 98.5%.

### Example 78

### 1-(6-Chloro-7-methylamino-3,4-dihydro-2H-benzo[1,4]oxazine-2-carbonyl)-4-(4-fluoro-benzyl)-piperidine-4-carbonitrile

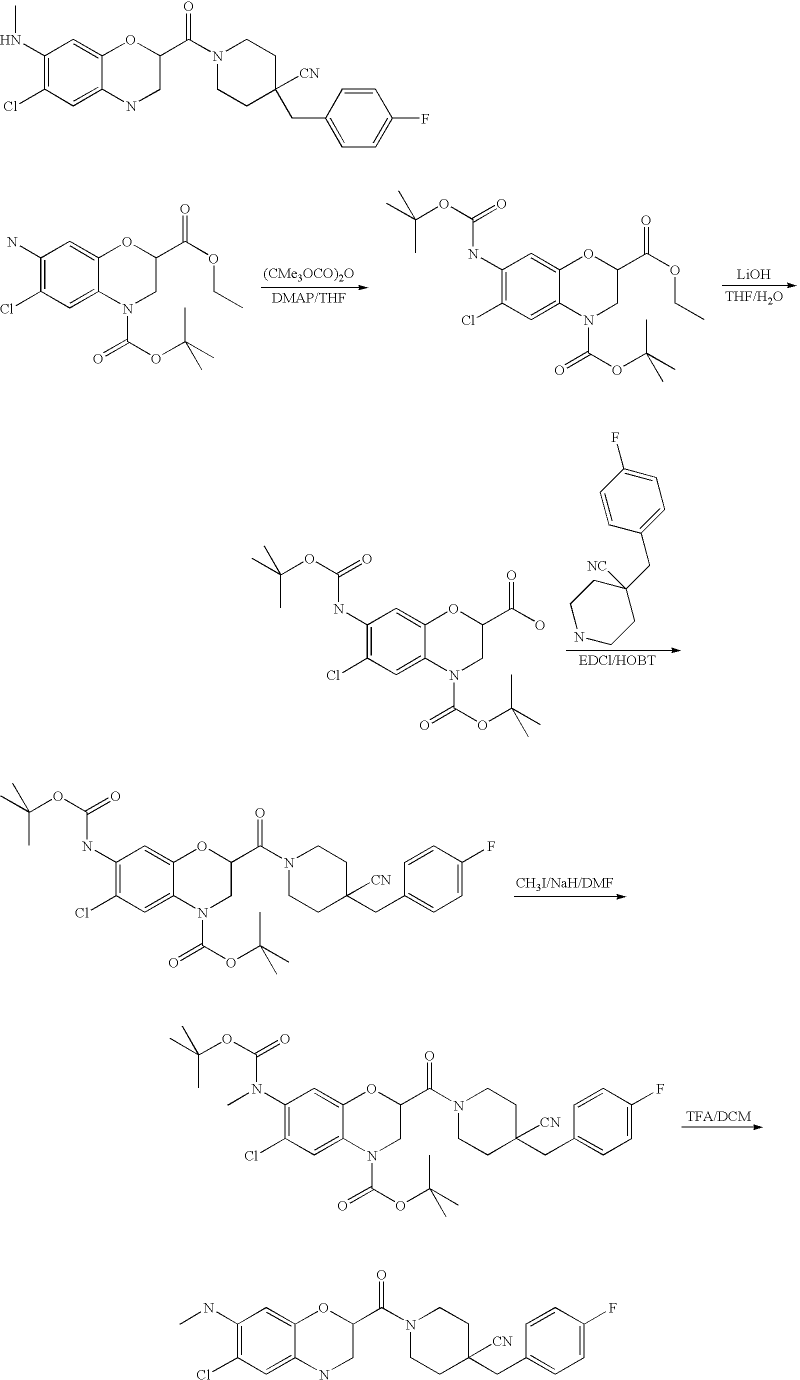

Step-1: 7-tert-Butoxycarbonylamino-6-chloro-2,3-dihydro-benzo[1,4]oxazine-2,4-dicarboxylic acid 4-tert-butyl ester 2-methyl ester

To a solution 7-Amino-6-chloro-2-(1-methoxy-vinyl)-2,3-dihydro-benzo[1,4]oxazine-4-carboxylic acid tert-butyl ester (400 mg, 1.12 mmol) in THF-1,4-dioxane was added ditertiary butyl dicarbonate (0.3 ml, 1.2 mmol) and DMAP (14 mg, 0.11 mmol). The reaction mixture was refluxed overnight. The reaction mixture was concentrated and purified over column chromatography (Si-gel, 3% ethyl acetate in hexane) to afford 110 mg (21.5%) of 7-tert-Butoxycarbonylamino-6-chloro-2,3-dihydro-benzo[1,4]oxazine-2,4-dicarboxylic acid 4-tert-butyl ester 2-methyl ester. ¹H-NMR (400 MHz, CDCl₃) δ (ppm): 7.83 (s, 1H), 6.83 (s, 1H), 4.70 (m, 1H), 4.35 (d, 1H), 3.60 (d, 1H), 1.50 (s, 3H), 1.43 (m, 2H), 1.45 (s, 9H).

Step-2: 7-tert-Butoxycarbonylamino-6-chloro-2,3-dihydro-benzo[1,4]oxazine-2,4-dicarboxylic acid 4-tent-butyl ester

To a solution 7-tert-Butoxycarbonylamino-6-chloro-2,3-dihydro-benzo[1,4]oxazine-2,4-dicarboxylic acid 4-tert-butyl ester 2-methyl ester (110 mg, 0.24 mmol) in THF-water was added LiOH (15 mg, 0.36 mmol) under ice cold condition and stirred at room temperature for 2 h. After completion of the reaction solvent was evaporated, added water, acidified with 50% acetic acid at 0° C. The organics was extracted with ethyl acetate, washed with water, brine, dried over Na₂SO₄. The organic layer was concentrated under reduced pressure to afford 100 mg (97.1%) of 7-tert-Butoxycarbonylamino-6-chloro-2,3-dihydro-benzo[1,4]oxazine-2,4-dicarboxylic acid 4-tert-butyl ester. ¹H-NMR (400 MHz, DMSO-d₆) δ (ppm): 13.3 (bs, 2H), 8.53 (s, 1H), 7.72 (s, 1H), 7.17 (s, 1H), 5.05(s, 1H), 4.40 (d, 1H), 3.57 (d, 1H), 1.42 (s, 18H).

Step-3: 7-tert-Butoxycarbonylamino-6-chloro-2-[4-cyano-4-(4-fluoro-benzyl)-piperidine-1-carbonyl]-2,3-dihydro-benzo[1,4]oxazine-4-carboxylic acid tert-butyl ester

To a solution of 7-tert-Butoxycarbonylamino-6-chloro-2,3-dihydro-benzo[1,4]oxazine-2,4-dicarboxylic acid 4-tert-butyl ester (0.1 g, 0.23 mmol) in DMF was added EDCI (0.058 g, 0.30 mmol), HOBT (0.016 g, 0.12 mmol) and DIPEA (0.1 ml, 0.69 mmol) at 0° C. The reaction mixture was stirred at room temperature for 1 h and was added 4-(4-Fluoro-benzyl)-piperidine-4-carbonitrile (0.051 g, 0.23 mmol) at 0° C. The reaction mixture was stirred at room temperature for overnight. The reaction mixture was diluted with ice-water and extracted with ethyl acetate. The organic layer was concentrated to afford 110 mg (73.9%) of 7-tert-Butoxycarbonylamino-6-chloro-2-[4-cyano-4-(4-fluoro-benzyl)-piperidine-1-carbonyl]-2,3-dihydro-benzo[1,4]oxazine-4-carboxylic acid tert-butyl ester. LC/MS [M+H]⁺:629.3.

Step-4: 7-(tert-Butoxycarbonyl-methyl-amino)-6-chloro-2-[4-cyano-4-(4-fluoro-benzyl)-piperidine-1-carbonyl]-2,3-dihydro-benzo[1,4]oxazine-4-carboxylic acid tert-butyl ester

To a solution E 7-tert-Butoxycarbonylamino-6-chloro-2-[4-cyano-4-(4-fluoro-benzyl)-piperidine-1-carbonyl]-2,3-dihydro-benzo[1,4]oxazine-4-carboxylic acid tert-butyl ester (110 mg, 0.17 mmol) in DMF was added sodium hydride (10 mg, 0.19 mmol) at 0° C. The reaction mixture was stirred at room temperature for 1 h. Methyl iodide (0.03 ml, 0.53 mmol) was added at 0° C. and the reaction mixture stirred at room temperature for 2 h. The reaction mixture was diluted with ice-water and extracted with ethyl acetate. The organic layer was washed with water and concentrated under reduced pressure. The crude was purified over column chromatography (Si-gel, 20% ethyl acetate in hexane) to afford 80 mg (73.2%) of 7-(tert-Butoxycarbonyl-methyl-amino)-6-chloro-2-[4-cyano-4-(4-fluoro-benzyl)-piperidine-1-carbonyl]-2,3-dihydro-benzo[1,4]oxazine-4-carboxylic acid tert-butyl ester. LC/MS [M+H]⁺: 643.4.

Step-5: 1-(6-Chloro-7-methylamino-3,4-dihydro-2H benzo[1,4]oxazine-2-carbonyl)-4-(4-fluoro-benzyl)-piperidine-4-carbonitrile

To a solution of 7-(tert-Butoxycarbonyl-methyl-amino)-6-chloro-2-[4-cyano-4-(4-fluoro-benzyl)-piperidine-1-carbonyl]-2,3-dihydro-benzo[1,4]oxazine-4-carboxylic acid tert-butyl ester (80 mg, 0.12 mmol) in DCM was added trifluoroacetic acid (1 ml, 12.9 mmol). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 25 mg (47%) of 1-(6-Chloro-7-methylamino-3,4-dihydro-2H benzo[1,4]oxazine-2-carbonyl)-4-(4-fluoro-benzyl)-piperidine-4-carbonitrile. LC/MS [M+H]⁺: 443.1. ¹H-NMR (400 MHz, DMSO-d₆) δ (ppm): 7.34 (bs, 2H), 7.19 (m, 2H), 6.57 (s, 1H), 6.09 (m, 1H), 5.13 (s, 1H), 4.90 (bs, 1H), 4.72 (bs, 1H), 4.40 (d, 1H), 4.09 (m, 1H), 3.15 (m, 2H), 2.95 (bs, 2H), 2.65 (s, 3H), 1.81(m, 2H), 1.54 (m, 1H), 1.39 (m, 2H), 1.14 (s, 1H). HPLC: 98.6%.

### Example 79

### 2-{6-Chloro-2-[4-cyano-4-(4-fluoro-benzyl)-piperidine-1-carbonyl]-7-methoxy-2,3-dihydro-benzo[1,4]oxazin-4-yl}-N-methyl-acetamide

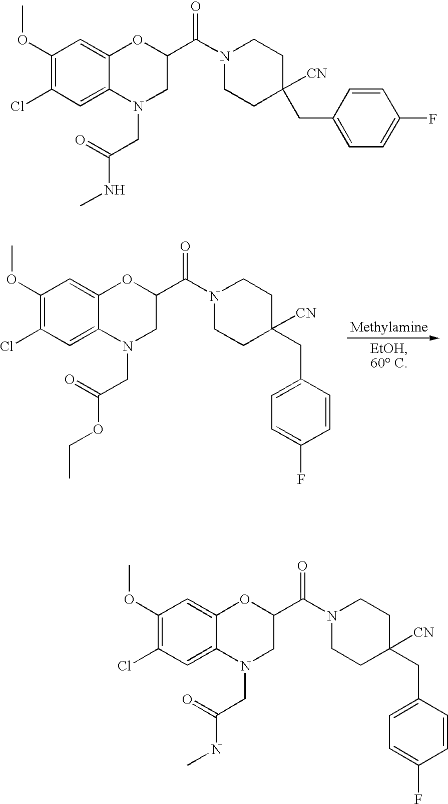

{6-Chloro-2-[4-cyano-4-(4-fluoro-benzyl)-piperidine-1-carbonyl]-7-methoxy-2,3-dihydro-benzo[1,4]oxazin-4-yl}-acetic acid ethyl ester was prepared by a process similar to that described in example 49.

A solution of {6-Chloro-2-[4-cyano-4-(4-fluoro-benzyl)-piperidine-1-carbonyl]-7-methoxy-2,3-dihydro-benzo[1,4]oxazin-4-yl}-acetic acid ethyl ester (80 mg, 0.1509 mmol) in ethanol was heated with methylamine solution of ethanol in sealed tube at 70° C. overnight. The ethanolic solution was concentrated and purified through column chromatography (Si-gel, 1.5% MeOH-DCM) to afford 50 mg (64.3%) of 2-{6-Chloro-2-[4-cyano-4-(4-fluoro-benzyl)-piperidine-1-carbonyl]-7-methoxy-2,3-dihydro-benzo[1,4]oxazin-4-yl}-N-methyl-acetamide. LC/MS [M+H]⁺: 515.4. ¹H-NMR (400 MHz, DMSO-d₆) δ (ppm): 7.97(m,1H), 7.34(m,2H), 7.19(m,2H), 6.66(m,1H), 6.51(s,1 H), 5.22(brs,1H), 4.39(m,1H), 4.11(m,1H), 3.78-1.53(m,18H). HPLC: 90.9%.

### Example 80

### 1-(6-Chloro-7-methoxy-3,4-dihydro-2H-benzo[1,4]oxazine-2-carbonyl)-4-(6-fluoro-pyridin-3-ylmethyl)-piperidine-4-carbonitrile

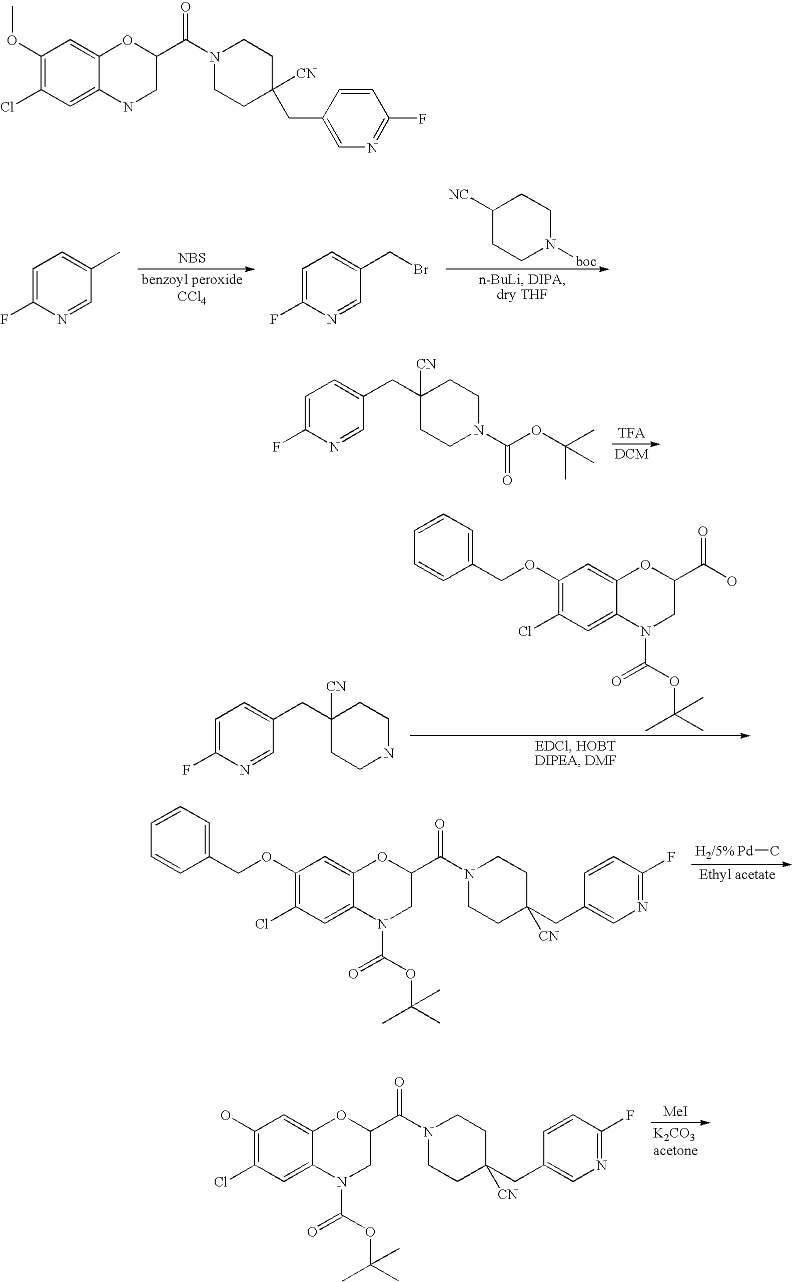

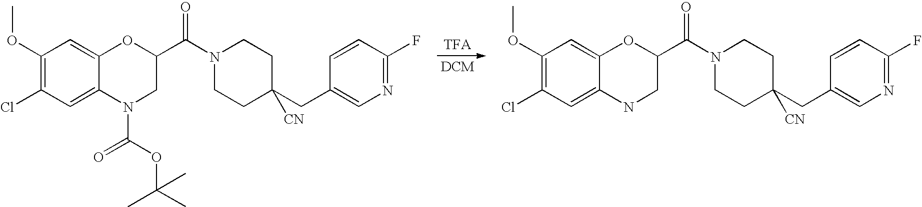

Step-1: 5-Bromomethyl-2-fluoro-pyridine

To a solution of 2-Fluoro-5-methyl-pyridine (500 mg, 4.5 mmol) in 10 ml of CCl₄ NBS (800 mg,4.5 mmol) & benzoyl peroxide (25 mg) was added in cold condition. The resulting mixture was allowed to stir at room temperature for 30 minutes and then refluxed for one hour. The reaction mass was cooled and diluted with water. The organic layer was separated and washed with sodium bicarbonate solution followed by water and brine, then dried over Na₂SO₄ and concentrated under vacuum. Purification of the crude mass over column chromatography (Si-gel, 5% ethyl acetate) afforded 350 mg (41%) of 5-Bromomethyl-2-fluoro-pyridine. GCMS (M/z): 189. ¹H-NMR (400 MHz, DMSO-d₆) δ (ppm): 8.34 (s, H), 8.04 (m, H), 7.21 (dd, H, J=2.6 Hz), 4.76 (s,2H).

Step-2: 4-Cyano-4-(6-fluoro-pyridin-3-ylmethyl)-piperidine-1-carboxylic acid tert-butyl ester

To a solution of of DIPEA (0.3 ml, 2.21 mmol) in dry THF, was added n-BuLi (2.58 mmol) solution dropwise at 0° C. The mixture was allowed to stir for 1 hr at room temperature. To this mixture a solution of N-boc-4-cyanopipyridine (387 mg, 1.84 mmol) in dry THF was added dropwise at −78° C. The resulting mixture was allowed to stir for two hours at −78° C. to 0° C. Then a solution of 5-Bromomethyl-2-fluoro-pyridine (350 mg, 1.84 mmol) in dry THF was added dropwise at −78° C. and the resulting mixture was allowed to stir at room temperature overnight. The reaction was quenched with saturated NH₄Cl solution in cold condition and stirred at room temperature for 30 minutes. THF layer was separated and the aqueous layer was extracted with ethyl acetate. The combined organic layers (THF and ethyl acetate) was washed with water and brine, then dried over Na₂SO₄ and concentrated under reduced pressure. Purification of the crude by column chromatography over silica gel (12% ethyl acetate-hexane) afforded 400 mg (68%) of 4-Cyano-4-(6-fluoro-pyridin-3-ylmethyl)-piperidine-1-carboxylic acid tert-butyl ester. ¹H-NMR (400 MHz, DMSO-d₆) δ (ppm): 8.16 (s, H), 7.92 (t, H, J=8.24Hz), 7.20 (d, H, J=8.36 Hz), 4.01 (d, 2H, J=13.36 Hz), 2.98 (s, 2H), 2.81(bs, 2H), 1.76 (d, 2H, J=13.28 Hz), 1.56 (m, 2H), 1.39 (s, 9H). LCMS [M+H]⁺:320.2.

Step 3: 4-(6-Fluoro-pyridin-3-ylmethyl)-piperidine-4-carbonitrile

To a solution of 4-Cyano-4-(6-fluoro-pyridin-3-ylmethyl)-piperidine-1-carboxylic acid tert-butyl ester (400 mg, 1.25 mmol) in DCM was added trifluoroacetic acid (0.97 ml, 12.52 mmol) dropwise in cold condition. The mixture was allowed to stir at room temperature for one and half hour. The reaction mixture was evaporated and the crude mass was diluted with water, basified with 2N NaOH solution and extracted with ethyl acetate. The organic layer was washed with water and brine, then dried over Na₂SO₄. The organic layer was concentrated under reduced pressure to afford 230 mg (84%) of 4-(6-Fluoro-pyridin-3-ylmethyl)-piperidine-4-carbonitrile. ¹H-NMR (400 MHz, DMSO-d₆) δ (ppm): 8.14 (s, H), 7.91 (t, H, J=8.2 Hz), 7.19 (d, H, J=8.36 Hz), 2.93 (s, 4H), 2.56 (m, 2H), 1.66 (d, 2H, J=12.6 Hz), 1.48 (m, 2H). LCMS [M+H]⁺: 220.2.

Step-4: 7-Benzyloxy-6-chloro-2-[4-cyano-4-(6-fluoro-pyridin-3-ylmethyl)-piperidine-1-carbonyl]-2,3-dihydro-benzo[1,4]oxazine-4-carboxylic acid tert-butyl ester

To a solution of 7-Benzyloxy-6-chloro-2,3-dihydro-benzo[1,4]oxazine-2,4-dicarboxylic acid 4-tert-butyl ester (440 mg, 1.05 mmol) in 1.5 ml of DMF was added EDCI (261.4 mg, 1.36 mmol), HOBT (71 mg, 0.52 mmol) and DIPEA (0.54 ml 3.14 mmol) at 10° C. The resulting reaction mixture was allowed to stir at room temperature for 30 minutes. Then a solution of 4-(6-Fluoro-pyridin-3-ylmethyl)-piperidine-4-carbonitrile in DMF (0.5 ml) was added to it at 10° C. and the reaction mixture was allowed to stir at room temperature overnight. The reaction mass was diluted with ice-water, stirred for 10 minutes and extracted with ethyl acetate. The organic layer was washed with water and brine, then dried over Na₂SO₄ and concentrated under vacuum. Purification of the crude organic mass by column chromatography (Si-gel, 0.2%MeOH-DCM) afforded 300 mg (46%) of 7-Benzyloxy-6-chloro-2-[4-cyano-4-(6-fluoro-pyridin-3-ylmethyl)-piperidine-1-carbonyl]-2,3-dihydro-benzo[1,4]oxazine-4-carboxylic acid tert-butyl ester. ¹H-NMR (400 MHz, DMSO-d₆) δ (ppm): 8.16 (d, H, J=15.76 Hz), 7.91 (m, H), 7.44 (m, 3H), 7.34 (s, H), 7.21 (d, 2H, J=8.2 Hz), 6.79 (d, H, J=16.08 Hz), 5.47(s, H), 5.16 (s, 2H), 4.34 (bs, H), 4.10 (bs, H), 3.58 (m, H), 3.14 (m, H), 3.02(s, 2H), 2.67(m, H), 1.86 (m, 2H), 1.74 (m, H), 1.56 (m, H), 1.39 (s, 9H), 1.24(s, H), 0.83 (m, H). LCMS [M+H]⁺: 621.3.

Step-5: 6-Chloro-2-[4-cyano-4-(6-fluoro-pyridin-3-ylmethyl)-piperidine-1-carbonyl]-7-hydroxy-2,3-dihydro-benzo[1,4]oxazine-4-carboxylic acid tert-butyl ester

7-Benzyloxy-6-chloro-2-[4-cyano-4-(6-fluoro-pyridin-3-ylmethyl)-piperidine-1-carbonyl]-2,3-dihydro-benzo[1,4]oxazine-4-carboxylic acid tert-butyl ester (300 mg, 0.48 mmol) was dissolved in 10 ml of ethyl acetate and hydrogenated over 5% Pd—C for 5 hrs. Then reaction mass was filtered through celite bed and the filtrate was concentrated under vacuum. Purification of the crude over column chromatography (Si-gel, 1% MeOH-DCM) afforded 200 mg (78%) of 6-Chloro-2-[4-cyano-4-(6-fluoro-pyridin-3-ylmethyl)-piperidine-1-carbonyl]-7-hydroxy-2,3-dihydro-benzo[1,4]oxazine-4-carboxylic acid tert-butyl ester. LCMS [M+H]⁺: 531.4. ¹H-NMR (400 MHz, DMSO-d₆) δ (ppm): 9.9 (bs, H), 8.27 (d, H, J=13.08 Hz), 7.92 (m, H), 7.55 (bs, H), 7.21 (d, H, J=6.6 Hz), 6.49 (d, H, J=7.36 Hz), 5.35 (d, H, J=26.68), 4.34 (d, H, J=12.56), 4.04 (m, H), 3.64 (d, H, J=13.26), 3.14 (m, H), 3.02 (s, H), 2.67(m, H), 1.86 (m, 2H), 1.84 (m, 2H), 1.56 (m, H), 1.43 (s, 9H), 1.24 (s, 2H), 0.83 (m, H).

Step-6: 6-Chloro-2-[4-cyano-4-(6-fluoro-pyridin-3-ylmethyl)-piperidine-1-carbonyl]-7-methoxy-2,3-dihydro-benzo[1,4]oxazine-4-carboxylic acid tert-butyl ester

To a solution of 6-Chloro-2-[4-cyano-4-(6-fluoro-pyridin-3-ylmethyl)-piperidine-1-carbonyl]-7-hydroxy-2,3-dihydro-benzo[1,4]oxazine-4-carboxylic acid tert-butyl ester (200 mg, 0.377 mmol) in acetone was added K₂CO₃ (156 mg, 1.13 mmol) and MeI (0.05 ml, 0.75 mmol) at 10° C. The resulting mixture was allowed to reflux for 3 hrs and concentrated. The crude was diluted with water and extracted with ethyl acetate. The organic layer was washed with water and brine, then dried over Na₂SO₄ and concentrated under vacuum. The crude was washed with hexane to get 195 mg (95%) of 6-Chloro-2-[4-cyano-4-(6-fluoro-pyridin-3-ylmethyl)-piperidine-1-carbonyl]-7-methoxy-2,3-dihydro benzo[1,4]oxazine-4-carboxylic acid tert-butyl ester. ¹H-NMR (400 MHz, DMSO-d₆) δ (ppm): 8.16 (d, H, J=14.2 Hz), 7.93 (m, H), 7.65 (bs, H), 7.22 (d, H, J=10.4 Hz), 6.69 (d, H, J=14.84 Hz), 5.35(d , H, J=26.68), 4.36 (d, H, J=13.6), 4.10 (d, H, J=13.88), 3.81 (s, 3H), 3.61 (d, H, J=12.6), 3.15 (m, H), 3.02(s, 2H), 2.67 (m, H), 1.89 (m, 3H), 1.52 (m, H), 1.45 (s, 9H), 1.23 (s, 2H), 0.84 (m, H). LCMS [M+H]⁺: 545.2.

Step-7: 1-(6-Chloro-7-methoxy-3,4-dihydro-2H-benzo[1,4]oxazine-2-carbonyl)-4-(6-fluoro-pyridin-3-ylmethyl)-piperidine-4-carbonitrile

To a solution of 6-Chloro-2-[4-cyano-4-(6-fluoro-pyridin-3-ylmethyl)-piperidine-1-carbonyl]-7-methoxy-2,3-dihydro-benzo[1,4]oxazine-4-carboxylic acid tert-butyl ester (190 mg, 0.35 mmol) in DCM was added trifluoroacetic acid (0.3 ml, 3.49 mmol) dropwise in cold condition. The mixture was allowed to stir at room temperature for one and half hour. Then DCM was evaporated and the crude mass was diluted with water, basified with 2N NaOH solution and extracted with ethyl acetate. The organic layer was washed with water and brine, then dried over Na₂SO₄. Concentration of the organic extract under reduced pressure afforded 150 mg (96%) of 1-(6-Chloro-7-methoxy-3,4-dihydro-2H-benzo[1,4]oxazine-2-carbonyl)-4-(6-fluoro-pyridin-3-ylmethyl)-piperidine-4-carbonitrile. LCMS [M+H]⁺: 445.2. ¹H-NMR (400 MHz, DMSO-d₆) δ (ppm): 8.18 (s, H), 7.94 (s, H), 7.22 (d, H, J=10.48 Hz), 6.62 (m,2H), 5.59(s, H,) 4.98 (d, H, J=13.6), 4.40 (bs, H), 4.04 (m, H), 3.72 (s, 3H), 3.37 (m, H), 3.21(m, 2H), 3.02 (s, 2H), 2.69 (m, H), 1.84 (m, 3H), 1.57 (m, 9H), 1.09 (s, 2H). HPLC: 90%.

### Example 81

### 6-Chloro-2-[4-cyano-4-(4-fluoro-benzyl)-piperidine-1-carbonyl]-7-methoxy-2,3-dihydro-benzo[1,4]oxazine-4-carbonitrile

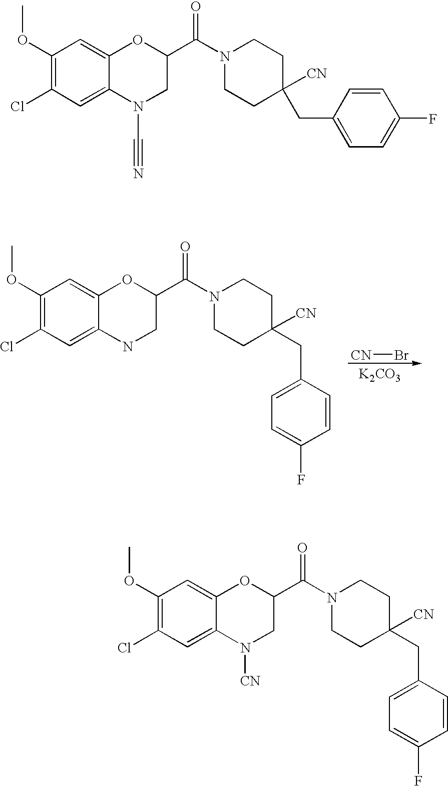

1-(6-Chloro-7-methoxy-3,4-dihydro-2H-benzo[1,4]oxazine-2-carbonyl)-4-(4-fluoro-benzyl)-piperidine-4-carbonitrile was prepared as described in example 27.

To a solution of 1-(6-Chloro-7-methoxy-3,4-dihydro-2H-benzo[1,4]oxazine-2-carbonyl)-4-(4-fluoro-benzyl)-piperidine-4-carbonitrile (50 mg, 0.11 mmol), in 1 ml of DMF was added K₂CO₃ (19 mg, 0.135 mmol) and the reaction mixture was stirred for half an hour. Then cyanogen bromide (14.3 mg, 0.135 mmol) was added to the previous reaction mixture at ice cool condition and the reaction mixture was stirred for 14 hours. Then the reaction mixture was diluted with water and extracted with ethyl acetate, washed with brine, dried over sodium sulfate and evaporated to get the crude mass which was further purified by column chromatography over silica gel (1.25% methanol in DCM) to afford 25 mg (47.7%) of 6-Chloro-2-[4-cyano-4-(4-fluoro-benzyl)-piperidine-1-carbonyl]-7-methoxy-2,3-dihydro-benzo[1,4]oxazine-4-carbonitile. LC/MS [M+H]⁺:469.2. ¹H-NMR (400 MHz, DMSO-d₆) δ (ppm): 7.35 (d, 2H), 7.19 (t, 2H), 7.03 (s, 1H), 6.84 (m, 1H), 5.38 (d,1H), 4.37 (d, 1H), 4.13 (m,1H), 3.90 (m, 2H), 3.79 (d, 3H), 3.26-1.39 (m, 8H). HPLC: 91.5%.

### Example 82

### 1-(6-Chloro-7-methoxy-3,4-dihydro-2H-benzo[1,4]oxazine-2-carbonyl)-4-(4-fluoro-benzyl)-piperidine-4-carboxylic acid ethyl ester

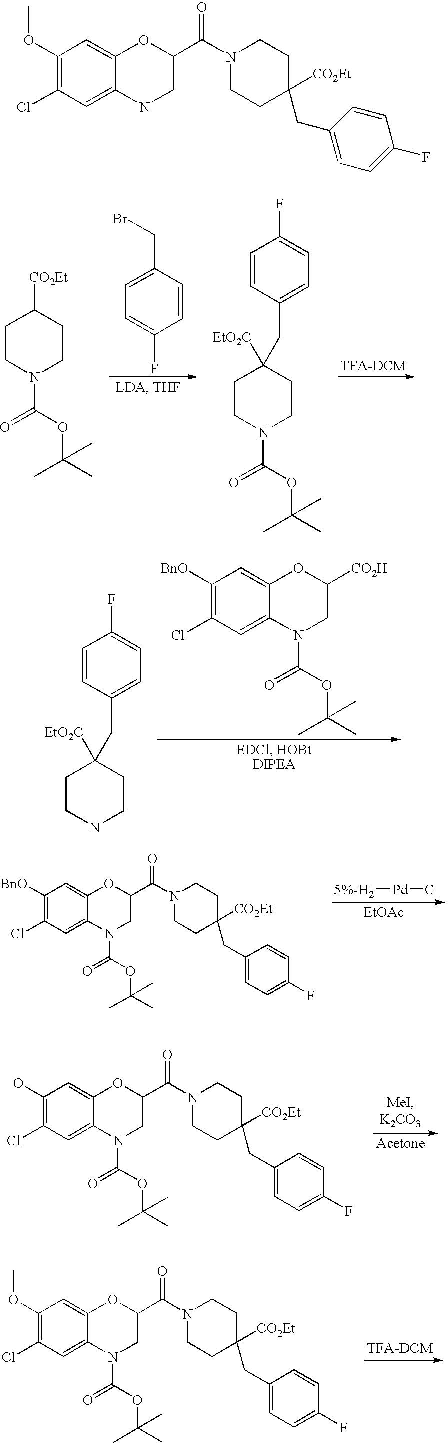

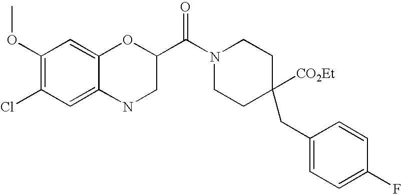

Step-1: 4-(4-Fluoro-benzyl)-piperidine-1,4-dicarboxylic acid 1-tent-butyl ester 4-ethyl ester

To a stirred solution of lithium diisopropyl amide in THF was added Piperidine-1,4-dicarboxylic acid 1-tert-butyl ester 4-ethyl ester (2 g, 7.7 mmol) at −78° C. The reaction mixture was stirred at −40° C. for 1 hr. Then reaction mixture was cooled to −78° C. and was added 4-fluorobenzyl bromide (1.46 g, 7.7 mmole) was added to reaction mass at −78° C. The resulting mixture was stirred at −78° C. for 2 hrs. Then reaction mixture was quenched with saturated ammonium chloride solution and extracted with ethyl acetate. Organic layer was washed with water and brine, dried over Na₂SO₄ and concentrated under reduced pressure. The crude mass was washed with hexane to afford 2.5 g (89.2%) of 4-(4-Fluoro-benzyl)-piperidine-1,4-dicarboxylic acid 1-tert-butyl ester 4-ethyl ester. LC/MS [M+H]⁺: 366.3.

Step-2: 4-(4-Fluoro-benzyl)-piperidine-4-carboxylic acid ethyl ester

To a solution of 4-(4-Fluoro-benzyl)-piperidine-1,4-dicarboxylic acid-1-tert-butyl ester 4-ethyl ester (1.0 g, 2.7 mmol) in DCM was added trifluoroacetic acid (2.47 g, 2.18 mmol) drop wise in cold condition. The reaction mixture was allowed to stir at room temperature for one and half an hour. The reaction mixture was evaporated and the crude mass was diluted with water, basified with 2N NaOH solution and extracted with ethyl acetate. The organic layer was washed with water and then dried over Na₂SO₄. Concentration of the organic compound under reduced pressure afforded 700 mg (97.7%) of compound 4-(4-Fluoro-benzyl)-piperidine-4-carboxylic acid ethyl ester. LC/MS [M+H]⁺: 266.4.

Step-3: 7-Benzyloxy-6-chloro-2-[4-ethoxycarbonyl-4-(4-fluoro-benzyl)-piperidine-1-carbonyl]-2,3-dihydro-benzo[1,4]oxazine-4-carboxylic acid tert-butyl ester

To a solution of 6-Chloro-7-ethoxy-2,3-dihydro-benzo[1,4]oxazine-2,4-dicarboxylic acid 4-tert-butyl ester (300 mg, 0.71 mmol) in DMF was added EDCI (178 mg, 0.93 mmol), HOBT (96.5 mg, 0.72 mmol) and DIPEA (276.9 mg, 2.14 mmol) at 10° C. The resulting reaction mixture was allowed to stir at room temperature for 30 minutes. Then a solution of 4-(4-Fluoro-benzyl)-piperidine-4-carboxylic acid ethyl ester (189.5 mg, 0.72 mmol) in DMF was added to it at 10° C. and the reaction mixture was allowed to stir at room temperature overnight. The reaction mixture was diluted with ice water, stirred for 10 minutes and extracted with ethyl acetate. The organic layer was washed with water, brine and dried over Na₂SO₄. The organic layer was concentrated under reduced pressure. Purification of the crude mass by column chromatography (Si-gel, 25% ethylacetate-hexane) afforded 350 mg (73.4%) of 7-Benzyloxy-6-chloro-2-[4-ethoxycarbonyl-4-(4-fluoro-benzyl)-piperidine-1-carbonyl]-2,3-dihydro-benzo[1,4]oxazine-4-carboxylic acid tert-butyl ester. LC/MS [M+H]⁺: 667.2.

Step-4: 6-Chloro-2-[4-ethoxycarbonyl-4-(4-fluoro-benzyl)-piperidine-1-carbonyl]-7-hydroxy-2,3-dihydro-benzo[1,4]oxazine-4-carboxylic acid tert-butyl ester

To a solution of 7-Benzyloxy-6-chloro-2-[4-ethoxycarbonyl-4-(4-fluoro-benzyl)-piperidine-1-carbonyl]-2,3-dihydro-benzo[1,4]oxazine-4-carboxylic acid tert-butyl ester (350 mg, 0.52 mmol) in ethyl acetate (20 ml) was hydrogenated with 5% Pd—C for 3 hours. The reaction mixture was filtered and concentrated under reduced pressure to afford 300 mg (99%) of 6-Chloro-2-[4-ethoxycarbonyl-4-(4-fluoro-benzyl)-piperidine-1-carbonyl]-7-hydroxy-2,3-dihydro-benzo[1,4]oxazine-4-carboxylic acid tert-butyl ester. LC/MS [M+H]⁺: 577.4.

Step-5: 6-Chloro-2-[4-ethoxycarbonyl-4-(4-fluoro-benzyl)-piperidine-1-carbonyl]-7-methoxy-2,3-dihydro-benzo[1,4]oxazine-4-carboxylic acid tert-butyl ester

To a solution of 6-Chloro-2-[4-ethoxycarbonyl-4-(4-fluoro-benzyl)-piperidine-1-carbonyl]-7-hydroxy-2,3-dihydro-benzo[1,4]oxazine-4-carboxylic acid tert-butyl ester (300 mg, 0.519 mmol) in acetone was added K₂CO₃ (143.7 mg, 1.03 mmol) and methyl Iodide (0.1 ml, 1.5594 mmol). The resulting reaction mixture was heated at reflux for 12 hrs. The reaction mixture was concentrated, diluted with water and extracted with ethyl acetate. The organic layer was washed with water and brine, dried over Na₂SO₄ concentrated under reduced pressure to afford 150 mg (48.8%) of 6-Chloro-2-[4-ethoxycarbonyl-4-(4-fluoro-benzyl)-piperidine-1-carbonyl]-7-methoxy-2,3-dihydro-benzo[1,4]oxazine-4-carboxylic acid tert-butyl ester.

LC/MS [M+H]⁺: 591.

Step-6: 1-(6-Chloro-7-methoxy-3,4-dihydro-2H-benzo[1,4]oxazine-2-carbonyl)-4-(4-fluoro-benzyl)-piperidine-4-carboxylic acid ethyl ester

To a solution of 6-Chloro-2-[4-ethoxycarbonyl-4-(4-fluoro-benzyl)-piperidine-1-carbonyl]-7-methoxy-2,3-dihydro-benzo[1,4]oxazine-4-carboxylic acid tert-butyl ester (170 mg, 0.29 mol) in DCM was added trifluoroacetic acid (0.26 ml, 2.29 mmol) drop wise in cold condition. The mixture was allowed to stir at room temperature for one and half hour. The organic layer was evaporated and the crude mass was diluted with water, basified with 2N NaOH solution and extracted with ethyl acetate. The organic layer was washed with water and brine, and then dried over Na₂SO₄. Concentration of the organic extract under vacuum afforded 110 mg (78%) of 1-(6-Chloro-7-methoxy-3,4-dihydro-2H-benzo[1,4]oxazine-2-carbonyl)-4-(4-fluoro-benzyl)-piperidine-4-carboxylic acid ethyl ester. LC/MS [M+H]⁺: 491.4. ¹H-NMR (400 MHz, DMSO-d₆) δ (ppm): 7.10 (bs, 4H), 6.32(s, 1H), 6.56 (s, 1H), 5.56 (s, 1H), 4.93 (s, 1H), 4.08 (m, 3H), 3.91 (s, 1H), 3.70 (d, 3H), 3.07 (m, 2H), 2.82 (s, 2H), 2.66 (m, 1H), 1.98 (m, 2H), 1.58 (m, 2H), 1.15 (m, 3H), 0.93 (bs, 1H). HPLC: 98.3%.

### Example 83

### 1-(6-Chloro-7-dimethylamino-3,4-dihydro-2H-benzo[1,4]oxazine-2-carbonyl)-4-(4-fluoro-benzyl)-piperidine-4-carboxylic acid ethyl ester

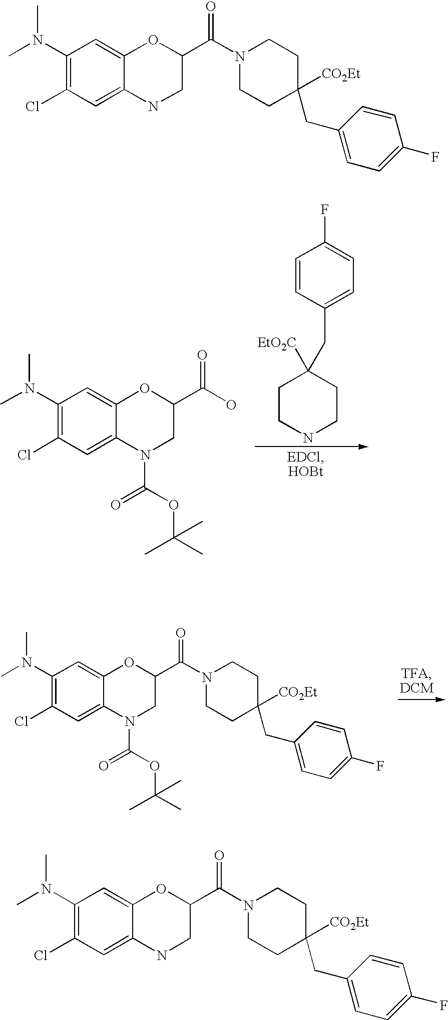

6-Chloro-7-dimethylamino-2,3-dihydro-benzo[1,4]oxazine-2,4-dicarboxylic acid 4-tert-butyl ester was prepared by a process as described in example 51.

4-(4-Fluoro-benzyl)-piperidine-4-carboxylic acid ethyl ester was prepared by a process similar to that described in example 82.

Step-1: 6-Chloro-7-dimethylamino-2-[4-ethoxycarbonyl-4-(4-fluoro-benzyl)-piperidine-1-carbonyl]-2,3-dihydro-benzo[1,4]oxazine-4-carboxylic acid tent-butyl ester

To a solution of 6-Chloro-7-dimethylamino-2,3-dihydro-benzo[1,4]oxazine-2,4-dicarboxylic acid 4-tert-butyl ester (300 mg, 0.84 mmol) in DMF was added 4-(4-Fluoro-benzyl)-piperidine-4-carboxylic acid ethyl ester (223 mg, 0.84 mmol), EDCI (209 mg, 1.09 mmol), HOBt (57 mg, 0.42 mmol) and DIPEA (0.4 ml, 2.5 mmol) at 5-10° C. The reaction mixture was stirred at room temperature for 15 hours then diluted with water and extracted with ethyl acetate. The organic layer was concentrated in vacuo and subjected to column chromatography over silica gel (3% MeOH-DCM) to afford 350 mg (69.1%) of 6-Chloro-7-dimethylamino-2-[4-ethoxycarbonyl-4-(4-fluoro-benzyl)-piperidine-1-carbonyl]-2,3-dihydro-benzo[1,4]oxazine-4-carboxylic acid tert-butyl ester. LC/MS [M+H]⁺: 604.1.

Step-2: 1-(6-Chloro-7-dimethylamino-3,4-dihydro-2H-benzo[1,4]oxazine-2-carbonyl)-4-(4-fluoro-benzyl)-piperidine-4-carboxylic acid ethyl ester

To a solution of 6-Chloro-7-dimethylamino-2-[4-ethoxycarbonyl-4-(4-fluoro-benzyl)-piperidine-1-carbonyl]-2,3-dihydro-benzo[1,4]oxazine-4-carboxylic acid tert-butyl ester (350 mg,0.57 mmol) in DCM was added trifluoroacetic acid (0.45 ml,5.7 mmol). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 285 mg (99%) of 1-(6-Chloro-7-dimethylamino-3,4-dihydro-2H-benzo[1,4]oxazine-2-carbonyl)-4-(4-fluoro-benzyl)-piperidine-4-carboxylic acid ethyl ester. LC/MS [M+H]⁺: 504.6. ¹H-NMR (400 MHz, DMSO-d₆) δ (ppm): 7.09 (d, 4H), 6.60 (bs, 1H), 6.56 (s, 1H), 5.71 (bs , 1H), 4.90 (bs, 1H), 4.13 (m, 1H), 4.08 (m, 2H), 3.90 (d, 1H), 3.21 (bs, H), 3.04 (m, 1H), 2.82 (s, 2H), 2.64 (m, 2H), 2.50 (s, 6H), 1.96 (m, 2H), 1.42 (m, 1H), 1.40 (m, 1H), 1.16 (s,3H).

HPLC: 96.3%.

### Example 84

### 1-(6-Chloro-7-methoxy-3,4-dihydro-2H-benzo[1,4]oxazine-2-carbonyl)-4-pyridin-4-ylmethyl-piperidine-4-carbonitrile

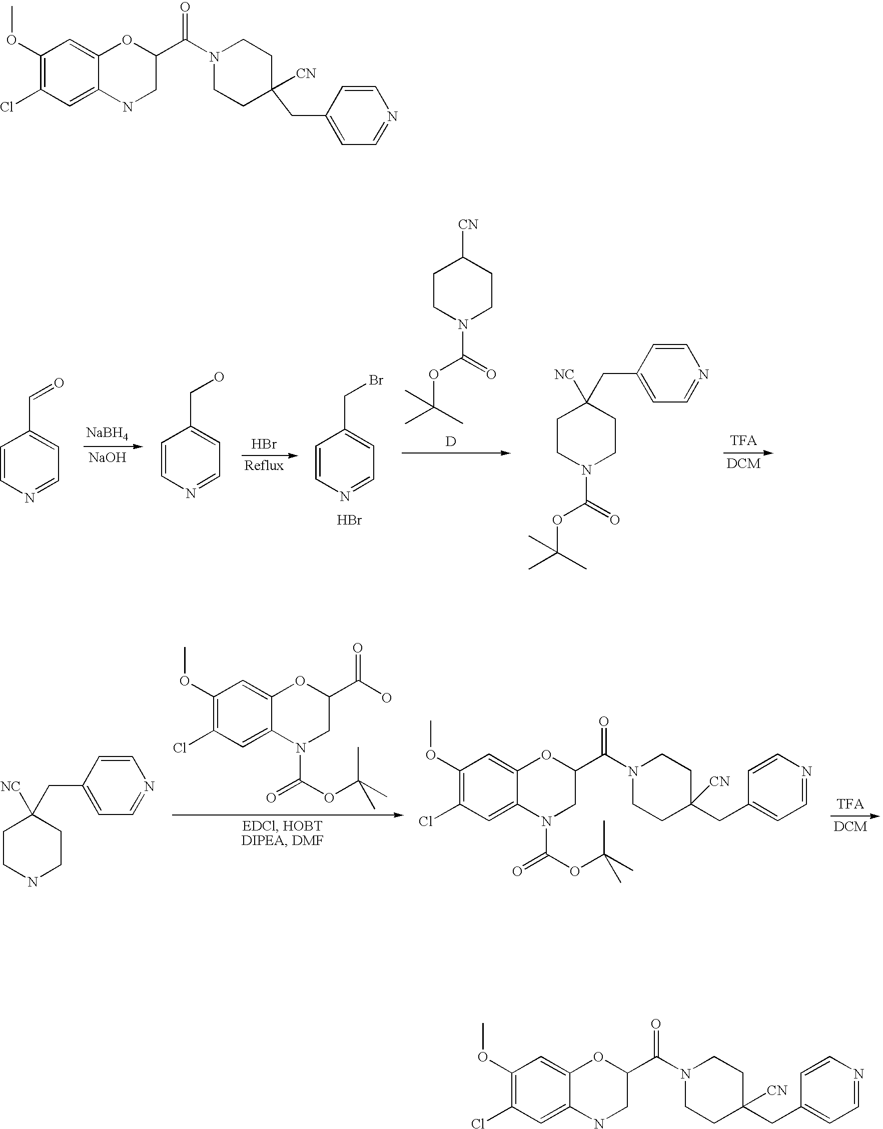

6-Chloro-7-methoxy-2,3-dihydro-benzo[1,4]oxazine-2,4-dicarboxylic acid4-tert-butyl ester was prepared by a process as described in example 27.
Step-1: Pyridin-4-yl-methanol

To a solution of Pyridine-4-carbaldehyde (500 mg, 4.66 mmol) in 10 ml of methanol was added NaBH₄ (172 mg, 4.66 mmol) in ice cool condition. The reaction mixture was stirred for half an hour in ice cool condition and then it was quenched with saturated solution of sodium sulfate and evaporated to a concentrated mass which was then diluted with water and extracted with 10% ethanol in ethyl acetate. The organic layer was dried over sodium sulfate and concentrated at reduced pressure to get 500 mg (98%) of Pyridin-4-yl-methanol. GCMS (m/z): 109.

Step-2: 4-Bromomethyl-pyridine hydrobromide

Pyridin-4-yl-methano (500 mg, 4.58 mmol) was taken in 5 ml of 48% of aqueous solution of HBr and the reaction mixture was subjected to reflux for 8 hours. Then the reaction mixture was evaporated, tripped with toluene and washed with hexane to afford 4-Bromomethyl-pyridine hydrobromide. GCMS (m/z): 172.

Step-3: 4-Cyano-4-pyridin-4-ylmethyl-piperidine-1-carboxylic acid tent-butyl ester

To a solution of DIPA (1 ml, 7.12 mmol) in THF was added n-BuLi (3.78 ml, 7.12 mmol) in ice cool condition. Then the reaction mixture was stirred for 1 hour at room temperature and was cooled to −78° C. To the previous solution at −78° C. was added a solution of 4-Bromomethyl-pyridine hydrobromide (749 mg, 3.56 mmol) in THF (5 ml) and stirred for another hour at −78° C. Then to that solution was added solid compound 4-fluorobenzyl bromide (900 mg, 3.56 mmol) at −78° C. and stirred at that temperature for 1 hour. The temperature of the reaction was allowed to rise at room temperature and stirred for 14 hours. Then the reaction mixture was quenched with saturated solution of ammonium chloride and extracted with ethyl acetate, washed with brine, dried over sodium sulfate and concentrated to get the crude mass which was further purified by column chromatography over silica gel (30% ethyl acetate-hexane) to afford 510 mg (47.4%) of 4-Cyano-4-pyridin-4-ylmethyl-piperidine-1-carboxylic acid tert-butyl ester.

LC/MS [M+H]⁺: 302.2.

Step-4: 4-Pyridin-4-ylmethyl-piperidine-4-carbonitrile

To a solution of 4-Cyano-4-pyridin-4-ylmethyl-piperidine-1-carboxylic acid tert-butyl ester (500 mg, 1.659 mmol) in DCM was added TFA (1.3 ml, 16.7 mmol) in ice cool condition and the reaction mixture was stirred for 2 hours. The reaction mixture was concentrated under reduced pressure and diluted with water, basified with 1(N) NaOH, then extracted with ethyl acetate. Organic layer was washed with water and brine, dried over sodium sulfate and concentrated under reduced pressure to get 225 mg (76%) of 4-Pyridin-4-ylmethyl-piperidine-4-carbonitrile. LC/MS [M+H]⁺: 202.2.

Step-5: 6-Chloro-2-(4-cyano-4-pyridin-4-ylmethyl-piperidine-1-carbonyl)-7-methoxy-2,3-dihydro-benzo[1,4]oxazine-4-carboxylic acid tert-butyl ester

To a solution of 6-Chloro-7-methoxy-2,3-dihydro-benzo[1,4]oxazine-2,4-dicarboxylic acid 4-tert-butyl ester (100 mg, 0.29 mmol) in DMF was added EDCI (72.5 mg, 0.38 mmol), HOBT (19.6 mg, 0.14 mmol) and DIPEA (0.15 ml, 0.87 mmol) was added at 5° C. The resulting mixture was allowed to stir at room temperature for 30 minutes. A solution of 4-Pyridin-4-ylmethyl-piperidine-4-carbonitrile (58.5 mg, 0.29 mmol) in DMF was added to it at 5° C. and the reaction mixture was allowed to stir at room temperature overnight. The reaction mass was diluted with ice-water, stirred for 10 minutes and extracted with ethyl acetate. The organic layer was washed with water and brine, then dried over Na₂SO₄ and concentrated under reduced pressure. Purification of the crude by column chromatography over silica gel (2% MeOH-DCM) as compound eluent afforded 90 mg (59%) of 6-Chloro-2-(4-cyano-4-pyridin-4-ylmethyl-piperidine-1-carbonyl)-7-methoxy-2,3-dihydro-benzo[1,4]oxazine-4-carboxylic acid tert-butyl ester. LC/MS [M+H]⁺: 527.2. ¹H-NMR (400 MHz, DMSO-d₆) δ (ppm): 8.56 (d, 2H), 7.65 (bs, H), 7.33 (m, 2H), 6.69 (d, H), 5.47 (m, H), 4.33 (bs, H), 4.12 (bs, 2H), 3.81 (s, 3H), 3.16 (m, H), 3.00 (m, 2H), 2.65 (m, H), 1.85 (m, 3H), 1.57 (m, H), 1.44 (s, 9H), 1.23 (s, H).

Step-6: 1-(6-Chloro-7-methoxy-3,4-dihydro-2H-benzo[1,4]oxazine-2-carbonyl)-4-pyridin-4-ylmethyl-piperidine-4-carbonitrile

To a solution of 6-Chloro-2-(4-cyano-4-pyridin-4-ylmethyl-piperidine-1-carbonyl)-7-methoxy-2,3-dihydro-benzo[1,4]oxazine-4-carboxylic acid tert-butyl ester (90 mg, 0.17 mmol) in DCM was added trifluoro acetic acid (0.13 ml, 1.71 mmol) dropwise in cold condition. The mixture was allowed to stir at room temperature 2 hours. Then DCM was evaporated and the crude mass was diluted with water and basified with 2N NaOH solution, then extracted with ethyl acetate. Ethyl acetate extract was washed with water and brine, dried over Na₂SO₄ and concentrated under reduced pressure. Washing of the crude mass with hexane afforded 55 mg (75%) of 1-(6-Chloro-7-methoxy-3,4-dihydro-2H-benzo[1,4]oxazine-2-carbonyl)-4-pyridin-4-ylmethyl-piperidine-4-carbonitrile. LC/MS [M+H]⁺: 427.2. ¹H-NMR (400 MHz, DMSO-d₆) δ (ppm): 11.91 (bs, H), 8.56 (d, 2H, J=5.0 Hz), 7.33 (s, 2H), 6.62 (m, 2H), 5.58 (s, H), 4.98 (bs, H), 4.39 (bs, H), 4.04 (m, H), 3.76 (s, 3H), 3.22 (m, 2H), 2.99 (s, 2H), 2.69 (m, H), 1.91 (s, H), 1.83 (m, 2H), 1.58 (m, H), 1.23 (m, H). HPLC: 96.4%.

### Example 85

### 1-(6-Chloro-7-dimethylamino-3,4-dihydro-2H-benzo[1,4]oxazine-2-carbonyl)-4-pyridin-4-ylmethyl-piperidine-4-carbonitrile

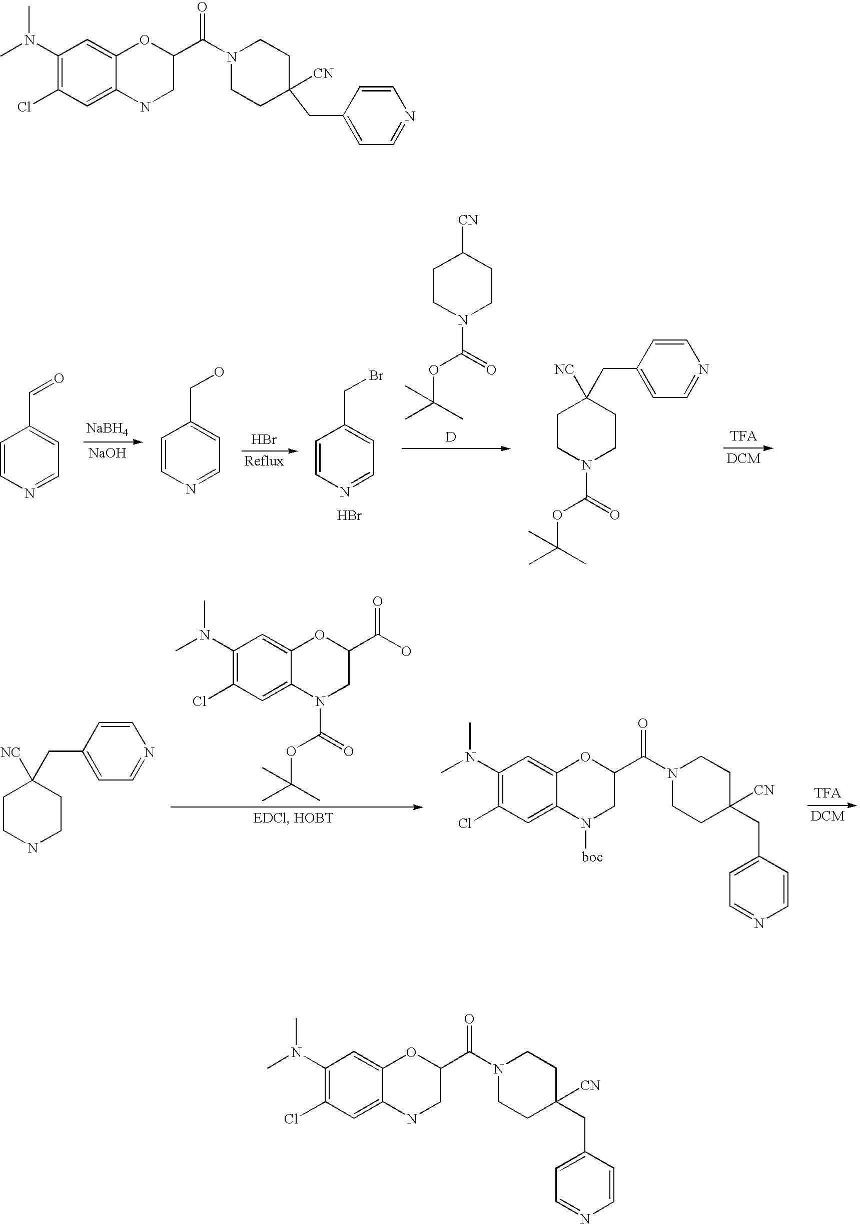

6-Chloro-7-dimethylamino-2,3-dihydro-benzo[1,4]oxazine-2,4-dicarboxylic acid 4-tert-butyl ester was prepared by a process as described in example 51.
Step-1: Pyridin-4-yl-methanol

To a solution of Pyridine-4-carbaldehyde (500 mg, 4.66 mmol), in 10 ml of methanol was added NaBH₄ (172 mg, 4.66 mmol) in ice cool condition. Then the reaction mixture was stirred for half an hour in ice cool condition and then it was quenched with saturated solution of sodium sulfate and evaporated to a concentrated mass which was then diluted with water and extracted with 10% ethanol in ethyl acetate. The organic layer was dried over sodium sulfate and concentrated at reduced pressure to get 500 mg (98%) of Pyridin-4-yl-methanol. GCMS (m/z): 109.

Step-2: 4-Bromomethyl-pyridine hydrobromide

Pyridin-4-yl-methano (500 mg, 4.58 mmol) was taken in 5 ml of 48% of aqueous solution of HBr and the reaction mixture was subjected to reflux for 8 hours. Then the reaction mixture was evaporated, tripped with toluene and washed with hexane to afford 4-Bromomethyl-pyridine hydrobromide. GCMS (m/z): 172.

Step-3: 4-Cyano-4-pyridin-4-ylmethyl-piperidine-1-carboxylic acid tert-butyl ester

To a solution of DIPA (1 ml, 7.12 mmol) in 5 ml of THF and to it was added n-BuLi (3.78 ml, 7.12mmol) in ice cool condition. The reaction mixture was stirred for 1 hour at room temperature and cooled to −78° C. To that solution was added a solution of 4-Bromomethyl-pyridine hydrobromide (749 mg, 3.56 mmol) in 5 ml of THF at −78° C. and stirred for another hour at −78° C. Then to that solution was added solid compound 4-fluorobenzyl bromide (900 mg, 3.56 mmol) at −78° C. and stirred at that temperature for 1 hour. The temperature of the reaction mixture was allowed to rise at room temperature and stirred for 14 hours. The reaction mixture was quenched with saturated solution of ammonium chloride and extracted with ethyl acetate, washed with brine, dried over sodium sulfate and concentrated to get the crude mass which was further purified by column chromatography (Si-gel, 30% ethyl acetate-hexane) to afford 510 mg (47.4%) of 4-Cyano-4-pyridin-4-ylmethyl-piperidine-1-carboxylic acid tert-butyl ester. LC/MS [M+H]⁺: 302.2.

Step-4: 4-Pyridin-4-ylmethyl-piperidine-4-carbonitrile

To a solution of 4-Cyano-4-pyridin-4-ylmethyl-piperidine-1-carboxylic acid tert-butyl ester (500 mg, 1.66 mmol) in DCM was added TFA (1.3 ml, 16.7 mmol) in ice cool condition and the reaction mixture was stirred for 2 hours. Then the reaction mixture was concentrated under reduced pressure and diluted with water, basified with 1(N) NaOH, then extracted with ethyl acetate. The organic layer was washed with water and brine, dried over sodium sulfate and concentrated under reduced pressure to afford 225 mg (76%) of 4-Pyridin-4-ylmethyl-piperidine-4-carbonitrile. LC/MS [M+H]⁺: 202.2

Step-5: 6-Chloro-2-(4-cyano-4-pyridin-4-ylmethyl-piperidine-1-carbonyl)-7-dimethylamino-2,3-dihydro-benzo[1,4]oxazine-4-carboxylic acid tert-butyl ester

To a solution of 6-Chloro-7-dimethylamino-2,3-dihydro-benzo[1,4]oxazine-2,4-dicarboxylic acid 4-tert-butyl ester (114 mg, 0.32 mmol) in 1 ml of DMF, EDCI (183 mg, 0.96 mmol), HOBT (22 mg, 0.16 mmol) and DIPEA (0.16 ml, 0.96 mmol) was added at 10° C. The resulting mixture was allowed to stir at room temperature for 30 minutes. Then a solution of 4-Pyridin-4-ylmethyl-piperidine-4-carbonitrile (64 mg, 0.32 mmol) in 0.5 ml DMF was added to it at 10° C. and the reaction mixture was allowed to stir at room temperature overnight. The reaction mass was diluted with ice-water, stirred for 10 minutes and extracted with ethyl acetate. Ethyl acetate layer was washed with water and brine, then dried over Na₂SO₄ and concentrated under vacuum. Purification of the crude by column chromatography over silica-gel (2-3% MeOH-DCM) afforded 100 mg (58%) of 6-Chloro-2-(4-cyano-4-pyridin-4-ylmethyl-piperidine-1-carbonyl)-7-dimethylamino-2,3-dihydro-benzo[1,4]oxazine-4-carboxylic acid tert-butyl ester. LC/MS [M+H]⁺: 540.3.

Step-6: 1-(6-Chloro-7-dimethylamino-3,4-dihydro-2H-benzo[1,4]oxazine-2-carbonyl)-4-pyridin-4-ylmethyl-piperidine-4-carbonitrile

To a solution of 6-Chloro-2-(4-cyano-4-pyridin-4-ylmethyl-piperidine-1-carbonyl)-7-dimethylamino-2,3-dihydro-benzo[1,4]oxazine-4-carboxylic acid tert-butyl ester (100 mg, 0.185 mmol) in DCM was added trifluoroacetic acid (0.14 ml, 1.85 mmol) drop wise at cold condition. The mixture was allowed to stir at room temperature for one and half an hour. Then DCM was evaporated and the crude mass was diluted with water, basified with 2N NaOH solution and extracted with ethyl acetate. The organic layer was washed with water and brine, and dried over Na₂SO₄. Concentration of the organic layer under reduced pressure afforded 50 mg (61%) of 1-(6-Chloro-7-dimethylamino-3,4-dihydro-2H-benzo[1,4]oxazine-2-carbonyl)-4-pyridin-4-ylmethyl-piperidine-4-carbonitrile. LC/MS [M+H]⁺: 442.8. ¹H-NMR (400 MHz, DMSO-d₆) δ (ppm): 8.56 (d, 2H, J=4.96), 7.33 (s, 2H), 6.61 (s, 1H), 6.57 (s, 1H), 5.74 (s, 1H), 4.95 (brs, 1H), 4.40 (m, 1H), 4.09 (m, 1H), 3.22 (m, 2H), 3.00 (brs, 2H), 2.70 (m, 1H), 2.57 (m, 6H), 1.83 (m, 4H), 1.58 (m, 1H). HPLC: 97.96%

### Example 86

### (6-Chloro-7-methoxy-3,4-dihydro-2H-benzo[1,4]oxazin-2-yl)-[4-(4-fluoro-benzyl)-4-hydroxymethyl-piperidin-1-yl]-methanone

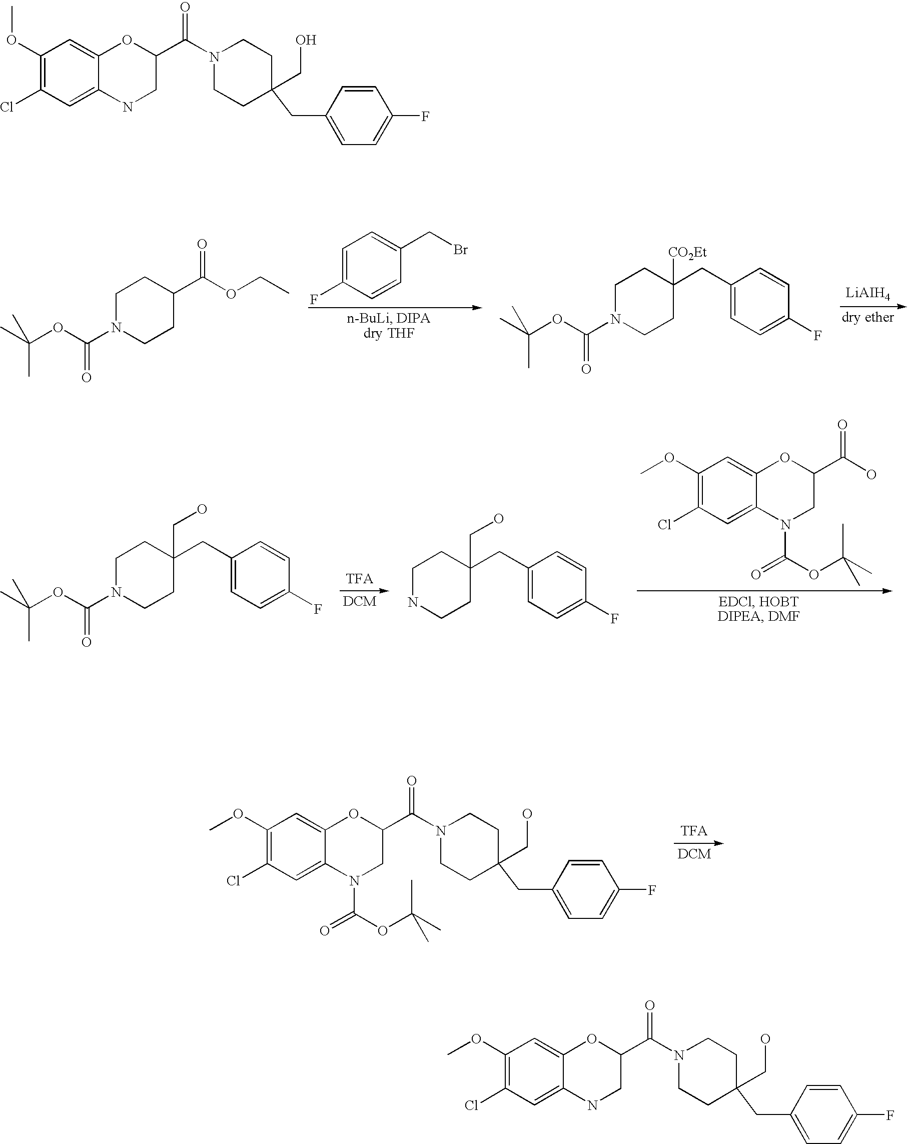

Step-1: 4-(4-Fluoro-benzyl)-piperidine-1,4-dicarboxylic acid 1-tert-butyl ester 4-ethyl ester

To a solution of of DIPEA (1.3 ml, 9.33 mmol) in dry THF, was added n-BuLi (10.88 mmol) solution dropwise at 0° C. The mixture was allowed to stir for 1 hr at room temperature. To this mixture a solution of Piperidine-1,4-dicarboxylic acid 1-tert-butyl ester 4-ethyl ester (2.0 g, 7.77 mmol) in dry THF was added dropwise at −78° C. The resulting mixture was allowed to stir for two hours at −78° C. to 0° C. Then a solution of 4-Fluoro benzylbromide (0.97 ml, 7.77 mmol) in dry THF was added dropwise at −78° C. and the resulting mixture was allowed to stir at room temperature overnight. The reaction was quenched with saturated NH₄Cl solution in cold condition and stirred at room temperature for 30 minutes. THF layer was separated and the aqueous layer was extracted with ethyl acetate. The combined organic layers (THF and ethyl acetate) was washed with water and brine, then dried over Na₂SO₄and concentrated under reduced pressure Purification of the crude by column chromatography (Si-gel, 4% ethyl acetate-hexane) afforded 2.3 g (81%) of 4-(4-Fluoro-benzyl)-piperidine-1,4-dicarboxylic acid 1-tert-butyl ester 4-ethyl ester. LC/MS [M+H]⁺: 366.3. ¹H-NMR (400 MHz, DMSO-d₆) δ (ppm): 7.08 (d, 4H, J=7.28 Hz), 4.05 (q, 2H, J=7.04 Hz, 14.12 Hz), 3.79 (d, 2H, J=13.16 Hz), 2.79 (s, 2H), 2.75 (m, 2H), 1.88 (d, 2H, J=13.32 Hz), 1.42 (m, 2H), 1.38 (s, 9H), 1.13 (t, 3H, J=7.08 Hz, 9.44 Hz).

Step-2: 4-(4-Fluoro-benzyl)-4-hydroxymethyl-piperidine-1-carboxylic acid tent-butyl ester

To a slurry of lithium aluminum hydride (57 mg, 1.5 mmol) in 6 ml of dry diethyl ether was added a solution of 4-(4-Fluoro-benzyl)-piperidine-1,4-dicarboxylic acid 1-tert-butyl ester 4-ethyl ester (500 mg, 1.37 mmol) in dry diethyl ether dropwise at 0° C. with maintaining the temperature of the reaction mixture below 10° C. The resulting mixture was stirred at 0° C. for one hour and then quenched with successive addition of water (0.057 ml), 15% aqueous NaOH (0.057 ml) and water (0.114 ml). The resultant slurry was stirred for one hour and then filtered through a celite bed. The filtrate was dried over Na₂SO₄ and concentrated under reduced pressure to afford 4-(4-Fluoro-benzyl)-4-hydroxymethyl-piperidine-1-carboxylic acid tert-butyl ester. LC/MS [M+H]⁺: 324.3.

¹H-NMR (400 MHz, DMSO-d₆) δ (ppm): 7.21 (m, 2H), 7.08 (t, 2H, J=8.84 Hz, 8.72 Hz), 4.67 (t, H, J=4.8 Hz, 4.84 Hz), 3.41 (m, 2H), 3.26 (m, 2H), 3.12 (d, 2H, J=4.76 Hz), 2.61 (s, 2H), 2.75 (m, 2H), J=13.32 Hz), 1.37 (s, 9H), 1.24 (m, 3H).

Step-3 : [4-(4-Fluoro-benzyl)-piperidin-4-yl]-methanol

To a solution of compound 4-(4-Fluoro-benzyl)-4-hydroxymethyl-piperidine-1-carboxylic acid tert-butyl ester (430 mg, 1.33 mmol) in 6 ml of DCM was added trifluoro acetic acid (1.02 ml, 13.3 mmol) dropwise in cold condition. The mixture was allowed to stir at room temperature for one hour. The reaction mixture was evaporated and the crude mass was diluted with water, neutralized with 2N NaOH solution and extracted with ethyl acetate. The organic layer was washed with water and brine, and then dried over Na₂SO₄. Concentration of the organic layer under reduced pressure afforded 230 mg (77%) of [4-(4-Fluoro-benzyl)-piperidin-4-yl]-methanol. ¹H-NMR (400 MHz, DMSO-d₆) δ (ppm): 8.31 (s,H), 7.21 (t, 2H, J=7.6 Hz, 5.92 Hz), 7.12 (t, 2H, J=8.72 Hz, 8.56 Hz), 4.85 (s, H) 3.16 (m, 2H), 3.12 (m, H), 3.03 (m, 2H), 2.65 (s, 2H),1.5 (m, 2H), 1.41 (m, 2H), 1.23 (s, H). LC/MS [M+H]⁺: 224.2.

Step-4: 6-Chloro-2-[4-(4-fluoro-benzyl)-4-hydroxymethyl-piperidine-1-carbonyl]-7-methoxy-2,3-dihydro-benzo[1,4]oxazine-4-carboxylic acid tert-butyl ester

To a solution of 6-Chloro-7-methoxy-2,3-dihydro-benzo[1,4]oxazine-2,4-dicarboxylic acid 4-tert-butyl ester (231 mg, 0.67 mmol) in DMF was added EDCI (167 mg, 0.87 mmol), HOBT (45 mg, 0.34 mmol) and DIPEA (0.35 ml 2.01 mmol) at 5° C. The resulting mixture was allowed to stir at room temperature for 30 minutes. Then a solution of [4-(4-Fluoro-benzyl)-piperidin-4-yl]-methanol (150 mg, 0.67 mmol) in DMF (0.5 ml) was added to the previous reaction mixture at 5° C. and the reaction mixture was allowed to stir at room temperature overnight. The reaction mass was diluted with ice-water, stirred for 10 minutes and extracted with ethyl acetate. The organic layer was washed with water and brine, then dried over Na₂SO₄ and concentrated under reduced pressure. Purification of the crude by column chromatography (Si-gel, 1.5% MeOH-DCM) afforded 80 mg (23%) of 6-Chloro-2-[4-(4-fluoro-benzyl)-4-hydroxymethyl-piperidine-1-carbonyl]-7-methoxy-2,3-dihydro-benzo[1,4]oxazine-4-carboxylic acid tert-butyl ester. LC/MS [M+H]⁺: 549.3. ¹H-NMR (400 MHz, DMSO-d₆) δ (ppm): 7.23 (m, 2H), 7.09 (t, 2H, J=8.6 Hz, 8.35 Hz), 6.67 (s, H), 5.38 (s, H), 4.76 (m, H), 3.98 (m, 2H), 3.79 (s, 3H), 3.60 (m, 2H), 3.18 (s, 2H), 2.89 (m, 2H), 2.73 (s, H), 2.67 (s, H), 2.55 (m, H), 1.43 (s, 9H), 1.26 (m, 2H).

Step-5: (6-Chloro-7-methoxy-3,4-dihydro-2H-benzo[1,4]oxazin-2-yl)-[4-(4-fluoro-benzyl)-4-hydroxymethyl-piperidin-1-methanone

To a solution of 6-Chloro-2-[4-(4-fluoro-benzyl)-4-hydroxymethyl-piperidine-1-carbonyl]-7-methoxy-2,3-dihydro-benzo[1,4]oxazine-4-carboxylic acid tert-butyl ester (80 mg,0.146 mmol) in 4 ml of DCM was added trifluoro acetic acid (0.11 ml, 1.46 mmol) dropwise in cold condition. The mixture was allowed to stir at room temperature for 2 hours. Then DCM was evaporated and the crude mass was diluted with water, basified with 2N NaOH solution and extracted with ethyl acetate. Ethyl acetate extract was washed with water and brine, then dried over Na₂SO₄ and concentrated under reduced pressure. Purification of the crude by column chromatography in 100-200 silica mesh and 3.5% MeOH-DCM as compound eluent afforded 30 mg (46%) of (6-Chloro-7-methoxy-3,4-dihydro-2H-benzo[1,4]oxazin-2-yl)-[4-(4-fluoro-benzyl)-4-hydroxymethyl-piperidin-1-yl]-methanone. LC/MS [M+H]⁺:449.4. ¹H-NMR (400 MHz, DMSO-d₆) δ (ppm): 7.23 (bs, 2H), 7.09 (t, 2H, J=8.76 Hz, 8.6 Hz), 6.63 (s, H), 6.55 (bs, H), 5.56 (s, H), 4.91 (bs, H), 4.73 (bs, H), 3.70 (d, 3H, J=10.36 Hz), 3.52 (m, 4H), 3.17 (s, 3H), 2.66 (s, 2H), 1.30 (m, 4H). HPLC: 98.9%.

### Example 87

### (6-Chloro-7-dimethylamino-3,4-dihydro-2H-benzo[1,4]oxazin-2-yl)-[4-(4-fluoro-benzyl)-4-hydroxymethyl-piperidin-1-yl]-methanone

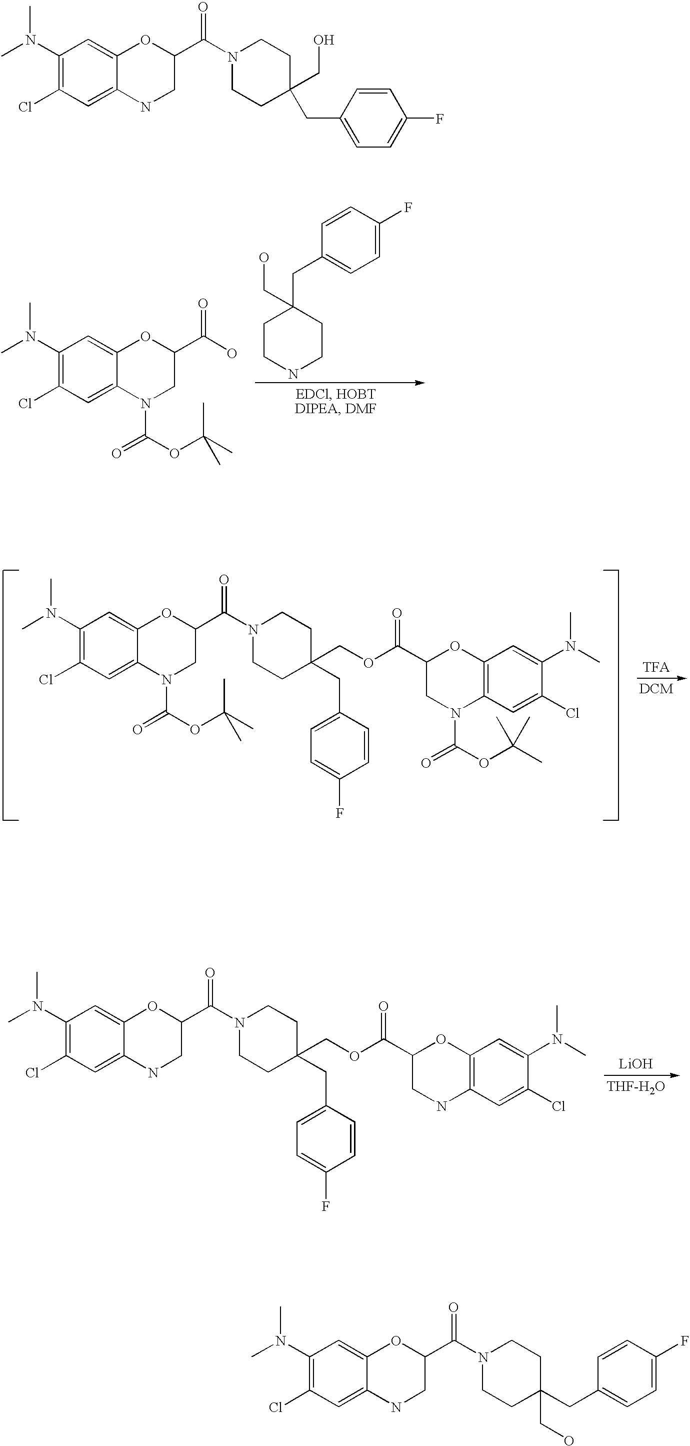

[4-(4-Fluoro-benzyl)-piperidin-4-yl]-methanol was prepared by a process as described in example 86.
Step-1: 6-Chloro-7-dimethylamino-3,4-dihydro-2H-benzo[1,4]oxazine-2-carboxylic acid 1-(6-chloro-7-dimethylamino-3,4-dihydro-2H-benzo[1,4]oxazine-2-carbonyl)-4-(4-fluoro-benzyl)-piperidin-4-ylmethyl ester

To a solution of 6-Chloro-7-dimethylamino-2,3-dihydro-benzo[1,4]oxazine-2,4-dicarboxylic acid 4-tert-butyl ester (150 mg, 0.42 mmol) in 0.5 ml of DMF, EDCI (104.8 mg, 0.55 mmol), HOBT (28.4 mg, 0.21 mmol) & DIPEA (0.22 ml 1.3 mmol) was added at 5° C. The resulting mixture was allowed to stir at room temperature for 30 minutes. Then a solution of [4-(4-Fluoro-benzyl)-piperidin-4-yl]-methanol* (94 mg, 0.42 mmol) in 0.3 ml DMF was added to it at 5° C. and the reaction mixture was allowed to stir at room temperature overnight. The reaction mass was diluted with ice-water, stirred for 10 minutes and extracted with ethyl acetate. Ethyl acetate layer was washed with water and brine, then dried over Na₂SO₄ and concentrated under reduced pressure. Purification of the crude by column chromatography (Si-gel, 2% MeOH-DCM) 130 mg of material showed desired mass as degradation. The material in DCM was added trifluoro acetic acid (0.75 ml, 9.7 mmol) dropwise in cold condition. The mixture was allowed to stir at room temperature for one hour. Then DCM was evaporated and the crude mass was diluted with water, basified with 2N NaOH solution and extracted with ethyl acetate. Ethyl acetate extract was washed with water and brine, then dried over Na₂SO₄ and concentrated under reduced pressure. Purification of the crude by column chromatography (Si-gel, 2% MeOH-DCM) afforded 60 mg (59%) of 6-Chloro-7-dimethylamino-3,4-dihydro-2H-benzo[1,4]oxazine-2-carboxylic acid 1-(6-chloro-7-dimethylamino-3,4-dihydro-2H-benzo[1,4]oxazine-2-carbonyl)-4-(4-fluoro-benzyl)-piperidin-4-ylmethyl ester. LC/MS [M+H]⁺: 700.6.

Step-3: (6-Chloro-7-dimethylamino-3,4-dihydro-2H-benzo[1,4]oxazin-2-yl)-[4-(4-fluoro-benzyl)-4-hydroxymethyl-piperidin-1-yl]-methanone

To a solution of 6-Chloro-7-dimethylamino-3,4-dihydro-2H-benzo[1,4]oxazine-2-carboxylic acid 1-(6-chloro-7-dimethylamino-3,4-dihydro-2H-benzo[1,4]oxazine-2-carbonyl)-4-(4-fluoro-benzyl)-piperidin-4-ylmethyl ester (60 mg,0.085 mmol) in THF-H₂O (3:1) was added LiOH.H₂O (7.2 mg, 0.171 mmol) in cold condition. The reaction mass was allowed to stir at room temperature for one hour, concentrated. The residue was diluted with water, and then extracted with ethyl acetate. The organic layer was washed with water and brine, then dried over Na₂SO₄ and concentrated under reduced pressure. Washing of the crude mass with hexane afforded 30 mg (76%) of (6-Chloro-7-dimethylamino-3,4-dihydro-2H-benzo[1,4]oxazin-2-yl)-[4-(4-fluoro-benzyl)-4-hydroxymethyl-piperidin-1-yl]-methanone. LC/MS [M+H]⁺: 462.1. ¹H-NMR (400 MHz, DMSO-d₆) δ (ppm): 7.23 (s, 2H), 7.09 (t, 2H, J=8.8 Hz, 8.64 Hz), 6.60 (s, H), 6.55 (bs, H), 5.74 (s,H), 4.90 (bs, H), 4.74 (s, H), 3.54 (m, 4H), 3.17 (s, 3H), 2.66 (s, 2H), 2.55 (s, 6H), 1.35 (m, 4H), 1.09 (m, H). HPLC: 97.5%.

### Example 88

### 6-Chloro-7-methoxy-3,4-dihydro-2H-benzo[1,4]oxazine-2-carboxylic acid [1-(4-fluoro-benzyl)-4-hydroxy-piperidin-4-ylmethyl]-amide

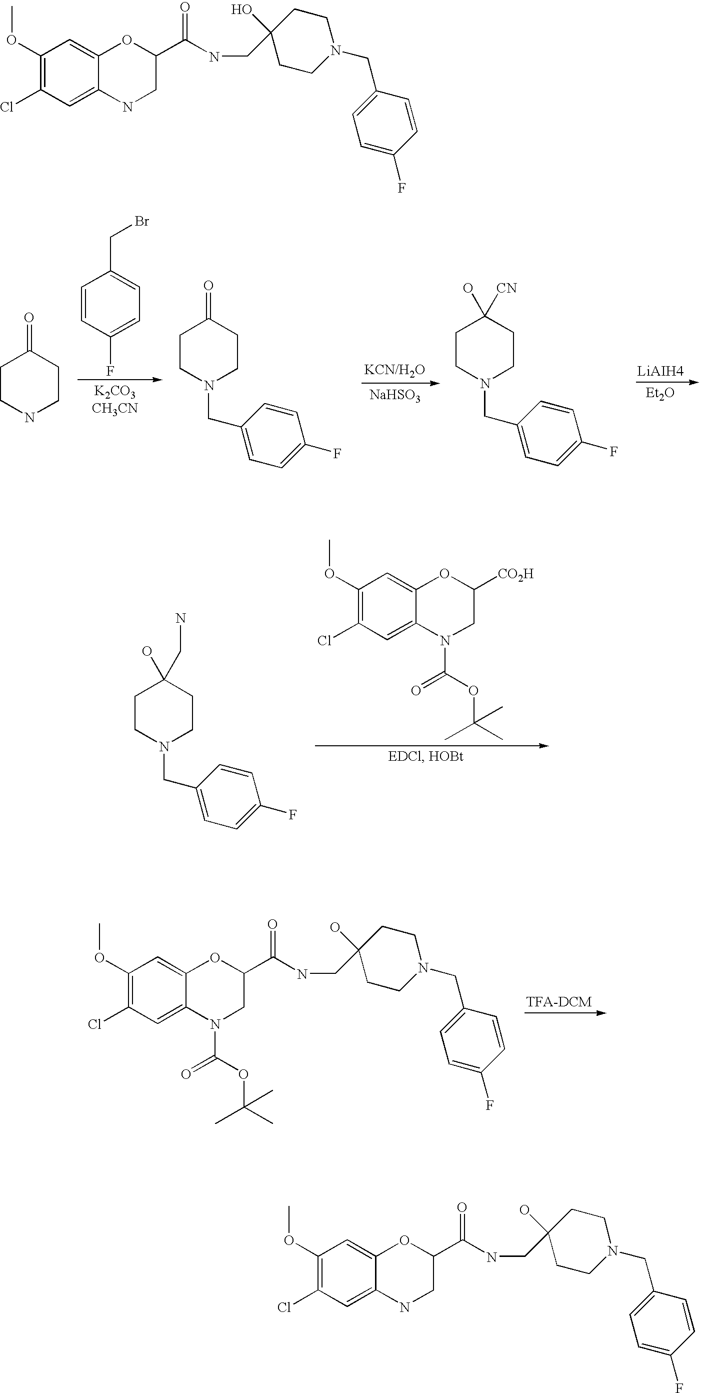

6-Chloro-7-methoxy-2,3-dihydro-benzo[1,4]oxazine-2,4-dicarboxylic acid 4-tert-butyl ester was prepared by a process as described in example 27.

### Step-1: 1-(4-Fluoro-benzyl)-piperidin-4-one

To a stirred solution of Piperidin-4-one (2 g, 13.0 mmol) in acetonitrile was added K₂CO_{3 (}5.40 g, 39.0 mmol) and 1-Bromomethyl-4-fluoro-benzene (1.5 ml, 11.7 mmol) at 0° C. The reaction mixture was stirred at room temperature for overnight. The reaction mixture was diluted with water and extracted with chloroform. The organic layer was washed with water, dilutes NaOH and dried over Na₂SO₄. The organic layer was concentrated in reduced pressure to afford 2.93 g of 1-(4-Fluoro-benzyl)-piperidin-4-one. LC/MS [M+H]⁺: 208.0.

Step-2: 1-(4-Fluoro-benzyl)-4-hydroxy-piperidine-4-carbonitrile

To a solution of 1-(4-Fluoro-benzyl)-piperidin-4-one (2.93 g, 10.9 mmol) in water was added potassium cyanide (2.6 g, 25.2 mmol) and aquous solution of NaHSO3 (2.6 g, 25.2 mmol). The reaction mixture was stirred for 1 hour. The reaction mixture was extracted with chloroform and washed with water. The organic layer was concentrated to afford 3.3 g of 1-(4-Fluoro-benzyl)-4-hydroxy-piperidine-4-carbonitrile. ¹H-NMR (400 MHz, CDCl₃) δ (ppm): 7.30 (m, H,),7.25 (m, H), 6.99(m,2H), 3.48 (s,2H,), 2.71 (m,2H), 2.55 (bs, H), 2.39 (m, 2H), 2.09 (m,2H), 1.87 (m, 2H).

Step-3: 4-Aminomethyl-1-(4-fluoro-benzyl)-piperidin-4-ol

To a solution of 1-(4-Fluoro-benzyl)-4-hydroxy-piperidine-4-carbonitrile (1 g, 4.2 mmol) in diethyl ether was added lithium aluminium hydride (324 mg, 8.5 mmol) portionwise. The reaction mixture was stirred at room temperature for 2 hours and quenched (0.3 mlwater, 0.3 ml 15% NaOH, 0.6 mlwater). The precipitated solid was filtered off through celite bed. The organic layer was concentrated under reduced pressure to afford 730 mg of 4-Aminomethyl-1-(4-fluoro-benzyl)-piperidin-4-ol. LC/MS [M+H]⁺: 239.4.

Step-4: 6-Chloro-2-{[1-(4-fluoro-benzyl)-4-hydroxy-piperidin-4-ylmethyl]-carbamoyl}-7-methoxy-2,3-dihydro-benzo[1,4]oxazine-4-carboxylic acid tert-butyl ester

To a solution of 6-Chloro-7-methoxy-2,3-dihydro-benzo[1,4]oxazine-2,4-dicarboxylic acid 4-tert-butyl ester (100 mg, 0.29 mmol) in DMF was added of 4-Aminomethyl-1-(4-fluoro-benzyl)-piperidin-4-ol (69 mg, 0.29 mmol), EDCI (72 mg, 0.38 mmol), HOBt (20 mg, 0.15 mmol) and DIPEA (0.15 ml, 0.87 mmol) at 5-10° C. The reaction mixture was stirred at room temperature for 16 hours. The reaction mixture was diluted with water and extracted with ethyl acetate. The organic layer was concentrated in vacuum and purified through column chromatography (Si-gel, 2% MeOH-DCM) to afford 60 mg (36.5%) of 6-Chloro-2-{[1-(4-fluoro-benzyl)-4-hydroxy-piperidin-4-ylmethyl]-carbamoyl}-7-methoxy-2,3-dihydro-benzo[1,4]oxazine-4-carboxylic acid tert-butyl ester. LC/MS [M+H]⁺: 564.4.

Step-5: 6-Chloro-7-methoxy-3,4-dihydro-2H-benzo[1,4]oxazine-2-carboxylic acid [1-(4-fluoro-benzyl)-4-hydroxy-piperidin-4-ylmethyl]-amide

To a solution of 6-Chloro-2-{[1-(4-fluoro-benzyl)-4-hydroxy-piperidin-4-ylmethyl]-carbamoyl}-7-methoxy-2,3-dihydro-benzo[1,4]oxazine-4-carboxylic acid tert-butyl ester (60 mg, 0.11 mmol)) in DCM was added trifluoroacetic acid (0.1 ml). The reaction mixture was stirred for about 5 h then concentrated and washed with dry ether. The resulting TFA salt was diluted with a minimum volume of water and neutralized with aq. NaOH solution at 5-10 ° C. Extraction of the neutralized solution with ethyl acetate followed by concentration afforded 40 mg (81.1%) of 6-Chloro-7-methoxy-3,4-dihydro-2H-benzo[1,4]oxazine-2-carboxylic acid [1-(4-fluoro-benzyl)-4-hydroxy-piperidin-4-ylmethyl]-amide. LC/MS [M+H]⁺: 464.44. ¹H-NMR (400 MHz, DMSO-d₆) δ (ppm): 7.61 (bs, 1H), 7.30 (t, 2H), 7.11 (t, 2H), 6.65 (d, 2H), 5.63 (s, 1H), 4.56 (m, 1H), 4.40 (s, 1H), 3.71 (s, 3H), 3.41 (m, 4H), 3.14 (m, 3H), 2.37 (bs, 2H), 2.28 (m, 2H), 1.98 (m, 1H), 1.67 (m, 1H), 1.40 (1H). HPLC: 92.9%.

### Example 89

### 6-Chloro-7-dimethylamino-3,4-dihydro-2H-benzo[1,4]oxazine-2-carboxylic acid [1-(4-fluoro-benzyl)-4-hydroxy-piperidin-4-ylmethyl]-amide

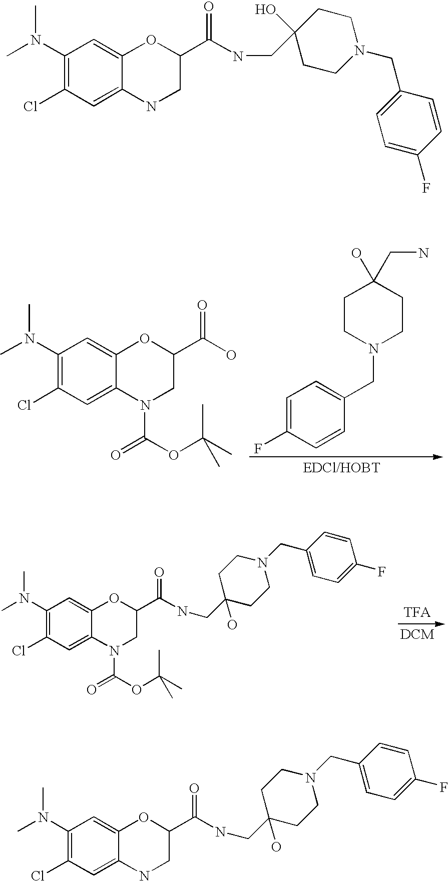

4-Aminomethyl-1-(4-fluoro-benzyl)-piperidin-4-ol was prepared by a process described in example 88.
Step 1: 6-Chloro-7-dimethylamino-2-{[1-(4-fluoro-benzyl)-4-hydroxy-piperidin-4-ylmethyl]- carbamoyl}-2,3-dihydro-benzo[1,4]oxazine-4-carboxylic acid tert-butyl ester

To a solution of 6-Chloro-7-dimethylamino-2,3-dihydro-benzo[1,4]oxazine-2,4-dicarboxylic acid 4-tert-butyl ester (67 mg,0.28 mmol) in DMF was added 4-Aminomethyl-1-(4-fluoro-benzyl)-piperidin-4-ol (100 mg,0.28 mmol), EDCI (69 mg,0.36 mmol), HOBT (19 mg,0.14 mmol) and DIPEA (0.15 ml, 0.84 mmol) at 5-10° C. The reaction mixture was stirred at room temperature for 15 hours then diluted with water and extracted with ethyl acetate. The ethyl acetate layer was concentrated in vacuo to afford 110 mg (68%) of 6-Chloro-7-dimethylamino-2-{[1-(4-fluoro-benzyl)-4-hydroxy-piperidin-4-ylmethyl]-carbamoyl}-2,3-dihydro-benzo[1,4]oxazine-4-carboxylic acid tert-butyl ester. LC/MS [M+H]⁺: 577.4.

Step-2: 6-Chloro-7-dimethylamino-3,4-dihydro-2H-benzo[1,4]oxazine-2-carboxylic acid[1-(4-fluoro-benzyl)-4-hydroxy-piperidin-4-ylmethyl]-amide

To a solution of 6-Chloro-7-dimethylamino-2-{[1-(4-fluoro-benzyl)-4-hydroxy-piperidin-4-ylmethyl]-carbamoyl}-2,3-dihydro-benzo[1,4]oxazine-4-carboxylic acid tert-butyl ester (110 mg,0.19 mmol) in DCM was added trifluoroacetic acid (0.3 ml, 3.9 mmol). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 53 mg (58.4%) of 6-Chloro-7-dimethylamino-3,4-dihydro-2H-benzo[1,4]oxazine-2-carboxylic acid [1-(4-fluoro-benzyl)-4-hydroxy-piperidin-4-ylmethyl]-amide. LC/MS [M+H]⁺: 477.4. ¹H-NMR (400 MHz, DMSO-d₆) δ (ppm): 7.60(m, H,),7.30(m,2H),7.11(m,2H), 6.77(s, H,), 6.62(s,H), 4.54 (m, H), 4.39 (s, H), 3.38 (m, 2H), 3.15 (m, 3H), 2.56 (s, 6H), 2.37 (m, 2H), 2.27 (m, 2H), 1.98 (m, H). HPLC: 93.3%.

### Example 90

### 1-(6-Chloro-7-dimethylamino-3,4-dihydro-2H-benzo[1,4]oxazine-2-carbonyl)-4-[(4-fluoro-phenyl)-hydroxy-methyl]-piperidine-4-carbonitrile

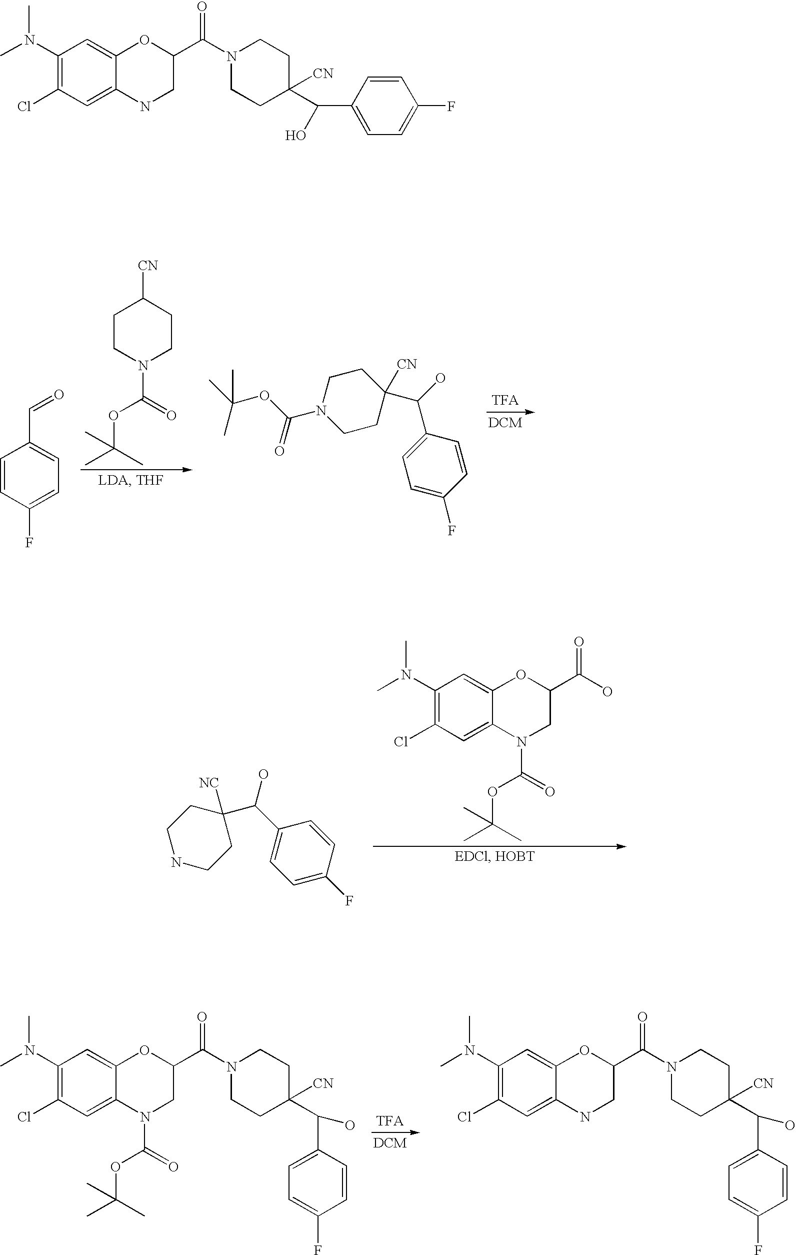

6-Chloro-7-dimethylamino-2,3-dihydro-benzo[1,4]oxazine-2,4-dicarboxylic acid 4-tert-butyl ester was prepared by a process as described in example 51.
Step-1: 4-Cyano-4-[(4-fluoro-phenyl)-hydroxy-methyl]-piperidine-1-carboxylic acid tert-butyl ester

To a solution of of DIPA (1.6 ml, 11.41 mmol) in dry THF, was added n-BuLi (13.31 mmol) solution dropwise at 0° C. The mixture was allowed to stir for 1 hr at room temperature. To this mixture a solution of N-boc-4-cyanopipyridine (2.0 g, 9.51 mmol) in dry THF was added dropwise at −78° C. The resulting mixture was allowed to stir for two hours at −78° C. to 0° C. Then a solution of 4-Fluoro benzaldehyde (1.1 ml, 9.51 mmol) in dry THF was added dropwise at −78° C. and the resulting mixture was allowed to stir at room temperature overnight. The reaction was quenched with saturated NH₄Cl solution in cold condition and stirred at room temperature for 30 minutes. THF layer was separated and the aqueous layer was extracted with ethyl acetate. The combined organic layers (THF and ethyl acetate) was washed with water and brine, then dried over Na₂SO₄ and concentrated under reduced pressure Purification of the crude mass by column chromatography(Si-gel, 20% ethyl acetate-hexane) afforded 1.2 g (38%) of 4-Cyano-4-[(4-fluoro-phenyl)-hydroxy-methyl]-piperidine-1-carboxylic acid tert-butyl ester. LC/MS [M+H]⁺: 335.2. ¹H-NMR (400 MHz, DMSO-d₆) δ (ppm): 7.43 (t, 2H, J=7.6 Hz, 5.72 Hz), 7.08 (t, 2H, J=8.32 Hz, 8.44 Hz), 4.51 (s, H), 4.14 (m, 2H), 2.95 (m, 2H), 2.29(s, H), 2.22 (d, H, J=13.44 Hz), 1.54(s, H), 1.49 (s, H), 1.43 (s, 9H).

Step-2: 4-[(4-Fluoro-phenyl)-hydroxy-methyl]-piperidine-4-carbonitrile

To a solution of 4-Cyano-4-[(4-fluoro-phenyl)-hydroxy-methyl]-piperidine-1-carboxylic acid tert-butyl ester (1.2 g, 3.59 mmol) in DCM was added trifluoro acetic acid (2.76 ml, 35.9 mmol) dropwise in cold condition. The mixture was allowed to stir at room temperature for one and half an hour. Then DCM was evaporated and the crude mass was diluted with water, neutralized with 2N NaOH solution and extracted with ethyl acetate. Ethyl acetate extract was washed with water and brine, and then dried over Na₂SO₄. Concentration of the organic extract under reduced pressure afforded 700 mg (83%) of 4-[(4-Fluoro-phenyl)-hydroxy-methyl]-piperidine-4-carbonitrile. LC/MS [M+H]⁺: 235.2. ¹H-NMR (400 MHz, DMSO-d₆) δ (ppm): 7.45 (t, 2H, J=6.8 Hz, 6.68 Hz), 7.19 (t, 2H, J=8.8 Hz, 8.68 Hz), 6.09 (s, H), 4.47 (s, H), 2.93 (d, H, J=12.28 Hz), 2.83 (d, H, J=12.64 Hz), 2.61 (t, H, J=12.4 Hz, 12.6 Hz), 2.02 (m, H), 1.45 (m, H), 1.14 (d, H, J=12.4 Hz).

Step-3: 6-Chloro-2-{4-cyano-4-1-[(4-fluoro-phenyl)-hydroxy-methyl]-piperidine-1-carbonyl}-7-dimethylamino-2,3-dihydro-benzo[1,4]oxazine-4-carboxylic acid tent-butyl ester

To a solution of 6-Chloro-7-dimethylamino-2,3-dihydro-benzo[1,4]oxazine-2,4-dicarboxylic acid 4-tert-butyl ester (100 mg, 0.280 mmol) in 1 ml of DMF, EDCI (161 mg, 0.84 mmol), HOBT (19mg, 0.14 mmol) and DIPEA (0.14 ml, 0.84 mmol) was added at 10° C. The resulting mixture was allowed to stir at room temperature for 30 minutes. Then a solution of 4-[(4-Fluoro-phenyl)-hydroxy-methyl]-piperidine-4-carbonitrile (66 mg, 0.28 mmol) in 0.5 ml DMF was added to it at 10° C. and the reaction mixture was allowed to stir at room temperature overnight. The reaction mass was diluted with ice-water, stirred for 10 minutes and extracted with ethyl acetate. Ethyl acetate layer was washed with water and brine, then dried over Na₂SO₄ and concentrated under vacuum. Purification of the crude mass by column chromatography (Si-gel, 2-3) % MeOH-DCM) afforded 110 mg (68%) of 6-Chloro-2-{4-cyano-4-[(4-fluoro-phenyl)-hydroxy-methyl]-piperidine-1-carbonyl -7-dimethylamino-2,3-dihydro-benzo[1,4]oxazine-4-carboxylic acid tert-butyl ester. LC/MS [M+H]⁺: 573.2.

Step-4: 1-(6-Chloro-7-dimethylamino-3,4-dihydro-2H-benzo[1,4]oxazine-2-carbonyl)-4-[(4-fluoro-phenyl)-hydroxy-methyl]-piperidine-4-carbonitrile

To a solution of 6-Chloro-2-{4-cyano-4-[(4-fluoro-phenyl)-hydroxy-methyl]-piperidine-1-carbonyl}-7-dimethylamino-2,3-dihydro-benzo[1,4]oxazine-4-carboxylic acid tert-butyl ester (100 mg, 0.174 mmol) in DCM was added trifluoroacetic acid (0.14 ml, 1.85 mmol) drop wise in cold condition. The mixture was allowed to stir at room temperature for one and half an hour. Then DCM was evaporated and the crude mass was diluted with water, basified with 2N NaOH solution and extracted with ethyl acetate. The organic layer was washed with water and brine and then dried over Na₂SO₄. Concentration of the organic layer under reduced pressure afforded 50 mg (61%) of 1-(6-Chloro-7-dimethylamino-3,4-dihydro-2H-benzo[1,4]oxazine-2-carbonyl)-4-[(4-fluoro-phenyl)-hydroxy-methyl]-piperidine-4-carbonitrile. LC/MS [M+H]⁺: 473.4. ¹H-NMR (400 MHz, DMSO-d₆) δ (ppm): 7.48(brs, 1H), 7.21(m,2H), 6.63(s,2H), 6.22(brs,1H), 4.93(m,1H), 4.46(m,1H), 4.02(m,1H), 3.24(m,1H), 2.64(brs,6H), 2.18 to 1.15(8H). HPLC: 98.4%.

### Example 91

### 1-[6-Chloro-7-dimethylamino-4-(2-dimethylamino-ethyl)-3,4-dihydro-2H-benzo[1,4]oxazine-2-carbonyl]-4-(4-fluoro-benzyl)-piperidine-4-carbonitrile

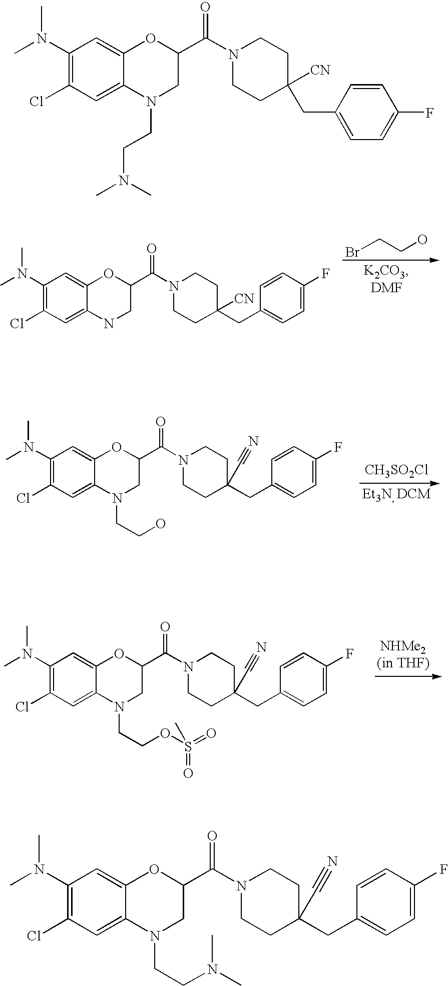

1-(6-Chloro-7-dimethylamino-3,4-dihydro-2H-benzo[1,4]oxazine-2-carbonyl)-4-(4-fluoro-benzyl)-piperidine-4-carbonitrile was prepared as described in example 51.
Step-1: 1-[6-Chloro-7-dimethylamino-4-(2-hydroxy-ethyl)-3,4-dihydro-2H-benzo[1,4]oxazine-2-carbonyl]-4-(4-fluoro-benzyl)-piperidine-4-carbonitrile

To a solution of 1-(6-Chloro-7-dimethylamino-3,4-dihydro-2H-benzo[1,4]oxazine-2-carbonyl)-4-(4-fluoro-benzyl)-piperidine-4-carbonitrile (150 mg, 0.33 mmol) in 1.0 ml of DMF, was added K₂CO₃ (97 mg, 0.66 mmol) & 2-Bromo ethanol (0.075 ml, 1.069 mmol). The resulting mixture was heated at 55° C. for 36 hrs. Then reaction mass was diluted with water and extracted with ethyl acetate. Organic extract was washed with water and brine, dried over Na₂SO₄ and concentrated under reduced pressure. Purification of the crude over column chromatography (Si-gel, 1% MeOH-DCM) afforded 100 mg (61%) of 1-[6-Chloro-7-dimethylamino-4-(2-hydroxy-ethyl)-3,4-dihydro-2H-benzo[1,4]oxazine-2-carbonyl]-4-(4-fluoro-benzyl)-piperidine-4-carbonitrile. LC/MS [M+H]⁺: 501.4. ¹H-NMR (400 MHz, DMSO-d₆) δ (ppm): 7.34 (bs, 2H), 7.19 (t, 2H, J=8.6 Hz, 8.52 Hz), 6.73 (s, H), 6.62 (d, H, J=17.32 Hz), 4.99 (s, H), 4.71 (m, H), 4.38 (m, H), 4.10 (m, 2H), 3.55 (s, 2H), 3.43 (m, 2H), 2.95 (d, 2H, J=10.56 Hz), 2.69 (m, H), 2.58 (s, 6H), 1.82 (m, H), 1.78 (m, 2H), 1.70 (m, H), 1.54 (m, H).

Step-2: Methanesulfonic acid 2-{6-chloro-2-[4-cyano-4-(4-fluoro-benzyl)-piperidine-1-carbonyl]-7-dimethylamino-2,3-dihydro-benzo[1,4]oxazin-4-yl}-ethyl ester

To a solution of 1-[6-Chloro-7-dimethylamino-4-(2-hydroxy-ethyl)-3,4-dihydro-2H-benzo[1,4]oxazine-2-carbonyl]-4-(4-fluoro-benzyl)-piperidine-4-carbonitrile (100 mg,0.20 mmol) in 3 ml of DCM was added triethyl amine (0.06 ml, 0.40 mmol) and Mesyl chloride (0.02 ml, 0.24 mmol) in cold condition and the mixture was stirred at rt for 4 hrs. Then the reaction mass in DCM was washed with bicarbonate solution followed by water and brine, dried over Na₂SO₄ and concentrated under reduced pressure. The crude was washed with hexane to afford 130 mg of Methanesulfonic acid 2-{6-chloro-2-[4-cyano-4-(4-fluoro-benzyl)-piperidine-1-carbonyl]-7-dimethylamino-2,3-dihydro-benzo[1,4]oxazin-4-yl}-ethyl ester. LC/MS [M+H]⁺: 579.4.

Step-3 : 1-[6-Chloro-7-dimethylamino-4-(2-dimethylamino-ethyl)-3,4-dihydro-2H-benzo[1,4]oxazine-2-carbonyl]-4-(4-fluoro-benzyl)-piperidine-4-carbonitrile

A solution of 2-{6-chloro-2-[4-cyano-4-(4-fluoro-benzyl)-piperidine-1-carbonyl]-7-dimethylamino-2,3-dihydro-benzo[1,4]oxazin-4-yl}-ethyl ester (130 mg, 0.22 mmol) and 6 ml 0 f Dimethyl amine (2M solution in THF) was heated in a seal tube at 80° C. overnight. The reaction mixture was evaporated under reduced pressure. Purification of the crude over column chromatography (Si-gel, 2.5% MeOH-DCM) afforded 70 mg (59%) of 1-[6-Chloro-7-dimethylamino-4-(2-dimethylamino-ethyl)-3,4-dihydro-2H-benzo[1,4]oxazine-2-carbonyl]-4-(4-fluoro-benzyl)-piperidine-4-carbonitrile. LC/MS [M+H]⁺: 428.3. ¹H-NMR (400 MHz, DMSO-d₆) δ (ppm): 7.35 (bs, 2H), 7.19 (t, 2H, J=8.8 Hz), 6.87(s, H), 6.64 (d, H, J=19.8 Hz), 5.13 (s, H), 4.37 (d, H, J=13.44), 4.14 (t, H, J=15.48 Hz, 14.72 Hz), 3.53 (bs, 2H), 3.40 (m, H), 3.01 (s, 2H), 2.73 (s, H), 2.63 (m, 4H), 2.58 (s, 6H), 2.50 (s, 6H), 1.82 (m, 2H), 1.79 (m, H), 1.53 (m, H). HPLC: 97.7%.

### Example 92

### 1-[6-Chloro-4-(2-dimethylamino-ethyl)-7-methoxy-3,4-dihydro-2H-benzo[1,4]oxazine-2-carbonyl]-4-(4-fluoro-benzyl)-piperidine-4-carbonitrile

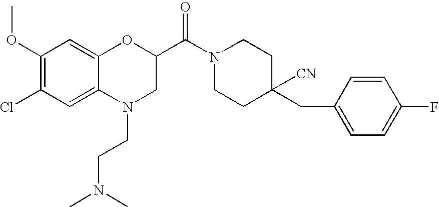

1-[6-Chloro-4-(2-dimethylamino-ethyl)-7-methoxy-3,4-dihydro-2H-benzo[1,4]oxazine-2-carbonyl]-4-(4-fluoro-benzyl)-piperidine-4-carbonitrile was prepared by a process similar to that described in example 91. LC/MS [M+H]⁺: 515.4. ¹H-NMR (400 MHz, DMSO-d₆) δ (ppm): 7.34 (bs, 2H), 7.19 (t, 2H), 6.78 (bs, 1H), 6.61 (m, 1H), 5.06 (m, 2H), 4.39 (d, 1H), 4.06 (m, 2H), 3.42 (s, 3H), 3.30 (m, 2H), 3.19 (m, 2H), 2.95 (s, 2H), 2.70 (s, 1H), 2.26 (bs, 6H), 1.90 (m, 2H), 1.74 (m, 1H), 1.53 (m, 1H), 1.23 (s, 1H). HPLC: 97.5%.

### Example 93

### (6-Chloro-7-methoxy-3,4-dihydro-2H-benzo[1,4]oxazin-2-yl)-[4-(4-fluoro-benzyl)-4-imidazol-1-ylmethyl-piperidin-1-yl]-methanone

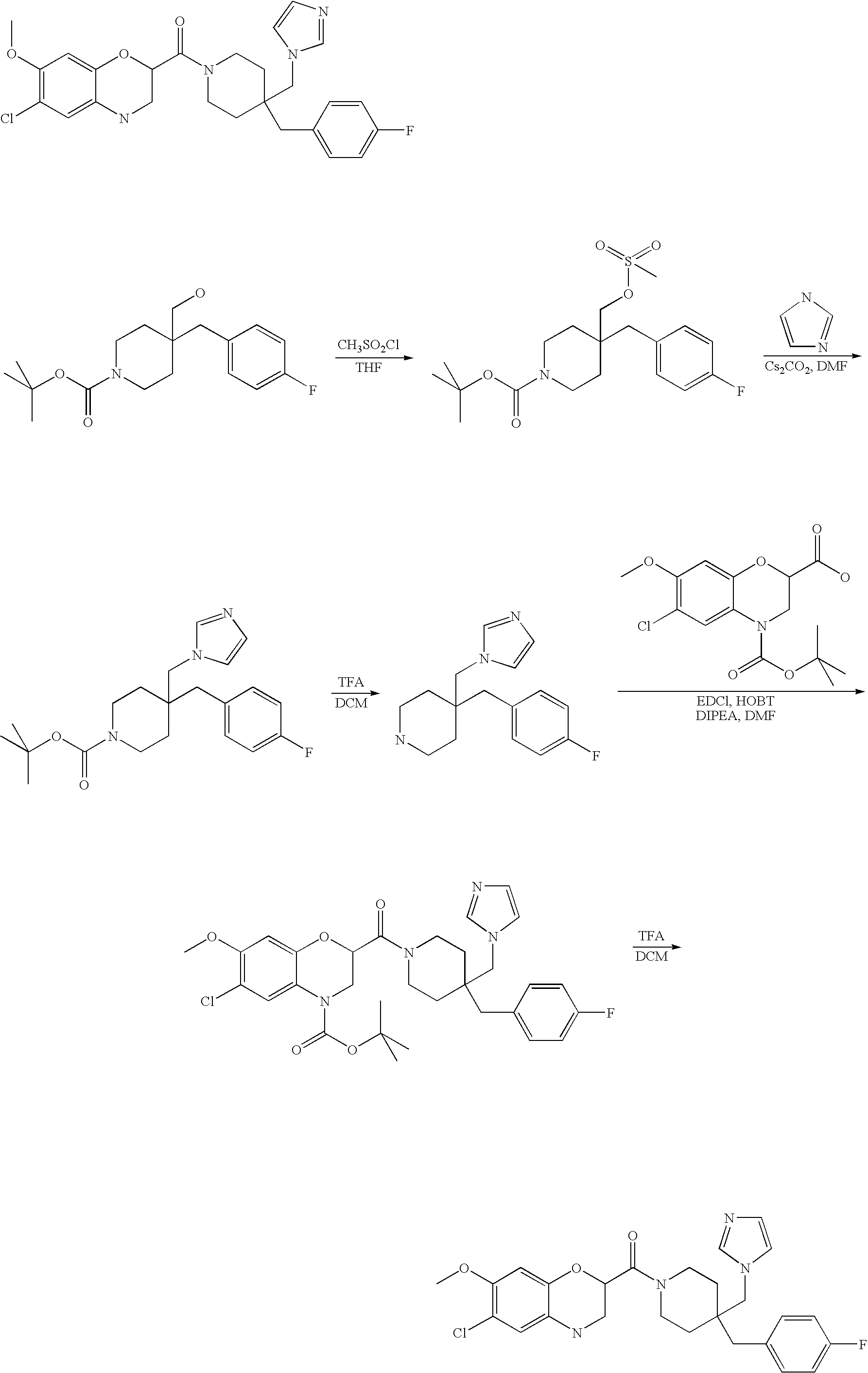

Step-1: 4-(4-Fluoro-benzyl)-4-methanesulfonyloxymethyl-piperidine-1-carboxylic acid tert-butyl ester

To a solution of 4-(4-Fluoro-benzyl)-4-hydroxymethyl-piperidine-1-carboxylic acid tert-butyl ester (400 mg,1.24 mmol) in DCM was added triethyl amine (0.52 ml, 3.71 mmol) followed by methane sulfonyl chloride (0.12 ml, 1.48 mmol) at cold condition. The reaction mixture was allowed to stir at room temperature for 5 hrs. Then the reaction mass was diluted with more DCM and washed with sodium bicarbonate solution followed by water and brine, dried over Na₂SO₄ and concentrated under reduced pressure. The crude mass thus obtained was washed with hexane and dried under reduced pressure to get 430 mg (86.6%) of 4-(4-Fluoro-benzyl)-4-methanesulfonyloxymethyl-piperidine-1-carboxylic acid tert-butyl ester. ¹H-NMR (400 MHz, DMSO-d₆) δ (ppm): 7.20 (t, 2H), 7.13 (t, 2H), 3.93 (s, 2H), 3.47 (m, 2H), 3.39 (m, H), 3.34 (m, H), 3.22 (s, 3H), 2.69 (s, 2H), 1.38 (s, 9H), 1.34 (m, 4H).

Step-2 : 4-(4-Fluoro-benzyl)-4-imidazol-1-ylmethyl-piperidine-1-carboxylic acid tert-butyl ester

To a solution of 4-(4-Fluoro-benzyl)-4-methanesulfonyloxymethyl-piperidine-1-carboxylic acid tert-butyl ester (400 mg, 1.27 mmol) in DMF was added Cs₂CO₃ (826 mg, 2.54 mmol). To this mixture, a solution of 1H-Imidazole (86 mg, 1.27 mmol) in 0.5 ml of DMF was added in cold condition and the resulting mixture was allowed to heat at 80° C. for 38 hrs. Then the reaction mass was cooled and diluted with water, extracted with ethyl acetate. The organic layer was washed with water and brine, dried over Na₂SO₄ and concentrated under reduced pressure. Purification of the crude by column chromatography (Si-gel, 1.5% MeOH-DCM) afforded 180 mg (38%) of 4-(4-Fluoro-benzyl)-4-imidazol-1-ylmethyl-piperidine-1-carboxylic acid tert-butyl ester. LC/MS [M+H]⁺: 374.2. ¹H-NMR (400 MHz, DMSO-d₆) δ (ppm): 7.58 (s, H), 7.15 (m, 5H), 6.89 (s, H), 5.76 (s, H), 3.98 (s, 2H), 3.42 (s, 4H), 2.67 (s, 2H) 1.31 (s, 9H), 1.15 (m, 3H).

Step-3: 4-(4-Fluoro-benzyl)-4-imidazol-1-ylmethyl-piperidine

To a solution of 4-(4-Fluoro-benzyl)-4-imidazol-1-ylmethyl-piperidine-1-carboxylic acid tert-butyl ester (180 mg,0.48 mmol) in 2 ml of DCM was added trifluoro acetic acid (0.37 ml, 4.82 mmol) dropwise in cold condition. The mixture was allowed to stir at room temperature for 90 minutes. The organic layer was evaporated and the crude mass was diluted with water, neutralized with 2N NaOH solution and extracted with ethyl acetate. The organic layer was washed with water, brine and dried over Na₂SO₄. Concentration of the organic layer under reduced pressure afforded 120 mg (91%) of 4-(4-Fluoro-benzyl)-4-imidazol-1-ylmethyl-piperidine. LC/MS [M+H]⁺: 274.4. ¹H-NMR (400 MHz, DMSO-d₆) δ (ppm): 7.55 (s, H), 7.12 (m, 5H), 6.89 (s, H), 3.93 (s, 2H), 2.77 (bs, 4H), 2.63 (s, 2H) 1.23 (m, 2H), 1.15 (m, 2H).

Step-4: 6-Chloro-2-[4-(4-fluoro-benzyl)-4-imidazol-1-ylmethyl-piperidine-1-carbonyl]-7-methoxy-2,3-dihydro-benzo[1,4]oxazine-4-carboxylic acid tert-butyl ester

To a solution of 6-Chloro-7-methoxy-2,3-dihydro-benzo[1,4]oxazine-2,4-dicarboxylic acid-4-tert-butyl ester (100 mg, 0.29 mmol) in 0.5 ml of DMF was added EDCI (72.5 mg, 0.38 mmol), HOBT (19.6 mg, 0.145 mmol) and DIPEA (0.15 ml, 0.87 mmol) at 5° C. The resulting mixture was allowed to stir at room temperature for 30 minutes. Then a solution of 4-(4-Fluoro-benzyl)-4-imidazol-1-ylmethyl-piperidine (79.5 mg, 0.29 mmol) in DMF (0.3 ml) was added to it at 5° C. and the reaction mixture was allowed to stir at room temperature overnight. The reaction mass was diluted with ice-water, stirred for 10 minutes and extracted with ethyl acetate. Ethyl acetate layer was washed with water and brine, then dried over Na₂SO₄ and concentrated under reduced pressure. Purification of the crude by column chromatography (Si-gel, 1.5% MeOH-DCM) afforded 120 mg (69%) of 6-Chloro-2-[4-(4-fluoro-benzyl)-4-imidazol-1-ylmethyl-piperidine-1-carbonyl]-7-methoxy-2,3-dihydro-benzo[1,4]oxazine-4-carboxylic acid tert-butyl ester. LC/MS [M+H]⁺: 599.4. ¹H-NMR (400 MHz, DMSO-d₆) δ (ppm): 7.61 (s, 2H), 7.19 (m, 2H), 7.13 (m, 3H), 6.91 (d, H, J=6.4 Hz), 6.60 (d, H, J=4.48 Hz), 5.28 (bs, H), 4.03 (d, 2H, J=14.56 Hz), 3.89 (m, H), 3.79 (s, 3H), 3.65 (m, 4H), 3.47 (m, H), 2.74 (s, H), 2.68 (m, H), 1.42 (s, 9H), 1.26 (m, 4H), 0.84 (m, H).

Step-5: (6-Chloro-7-methoxy-3,4-dihydro-2H-benzo[1,4]oxazin-2-yl)-[4-(4-fluoro-benzyl)-4-imidazol-1-ylmethyl-piperidin-1-yl]-methanone

To a solution of 6-Chloro-2-[4-(4-fluoro-benzyl)-4-imidazol-1-ylmethyl-piperidine-1-carbonyl]-7-methoxy-2,3-dihydro-benzo[1,4]oxazine-4-carboxylic acid tert-butyl ester (110 mg,0.18 mmol) in 3 ml of DCM was added trifluoro acetic acid (0.14 ml, 1.84 mmol) dropwise in cold condition. The mixture was allowed to stir at room temperature for two hours. Then DCM was evaporated and the crude mass was diluted with water, basified with 2N NaOH solution and extracted with ethyl acetate. The organic layer was washed with water and brine, then dried over Na₂SO₄ and concentrated under reduced pressure. Purification of the crude by column chromatography (Si-gel, 3% MeOH-DCM) afforded 55 mg (60%) of (6-Chloro-7-methoxy-3,4-dihydro-2H-benzo[1,4]oxazin-2-yl)-[4-(4-fluoro-benzyl)-4-imidazol-1-ylmethyl-piperidin-1-yl]-methanone. LC/MS [M+H]⁺: 499.4. ¹H-NMR (400 MHz, DMSO-d₆) δ (ppm): 7.61 (s, H), 7.18 (m, 5H), 6.91 (d, H, J=4.44 Hz), 6.61 (s, H), 6.48 (d, H, J=7.04 Hz),5.54 (s, H), 4.89 (bs, H), 4.02 (d, 2H, J=21.96 Hz), 3.71 (s, 3H), 3.66 (m, 2H), 3.51 (m, H), 3.23 (m, H), 3.13 (m, H), 2.73 (m, 2H), 1.33 (m, 4H), 0.84 (m, H). HPLC: 98.3%.

### Example 94

### 1-(6-Chloro-7-ethyl-3,4-dihydro-2H-benzo[1,4]oxazine-2-carbonyl)-4-(4-fluoro-benzyl)-piperidine-4-carbonitrile

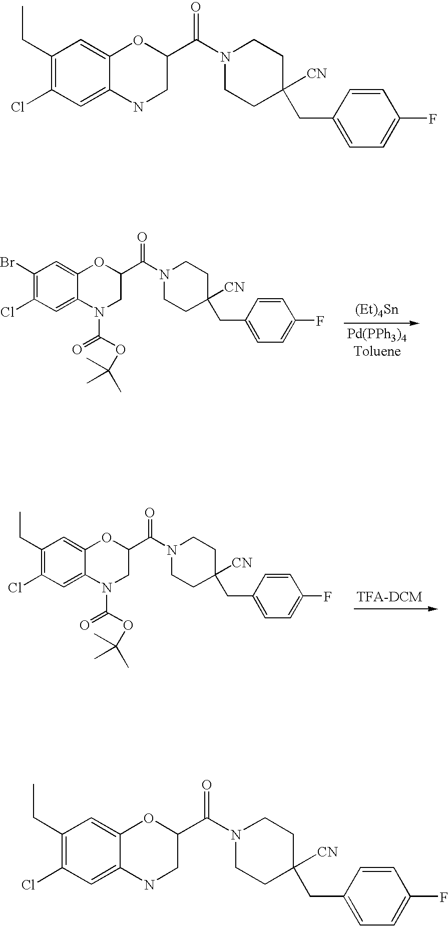

1-(7-Bromo-6-chloro-3,4-dihydro-2H-benzo[1,4]oxazine-2-carbonyl)-4-(4-fluoro-benzyl)-piperidine-4-carbonitrile was prepared by a process described in example 57.
Step-1: 6-Chloro-2-[4-cyano-4-(4-fluoro-benzyl)-piperidine-1-carbonyl]-7-ethyl-2,3-dihydro-benzo[1,4]oxazine-4-carboxylic acid tert-butyl ester

To a solution of 1-(7-Bromo-6-chloro-3,4-dihydro-2H-benzo[1,4]oxazine-2-carbonyl)-4-(4-fluoro-benzyl)-piperidine-4-carbonitrile (200 mg, 0.34 mmol) in toluene was degassed with argon and was added tetrabutyltin (79.2 mg, 0.34 mmol) and [Pd(PPh₃)₄] (1.57 mg, 0.002 mmol). The reaction mixture was refluxed overnight. The reaction mixture was concentrated and partitioned in 1:1 acetonitrile-hexane. The acetonitrile solution was concentrated under reduced pressure and crude was purified over column chromatography (Si-gel, 10% ethyl acetate-hexane) to afford 180 mg (97.7%) of 6-Chloro-2-[4-cyano-4-(4-fluoro-benzyl)-piperidine-1-carbonyl]-7-ethyl-2,3-dihydro-benzo[1,4]oxazine-4-carboxylic acid tert-butyl ester. LC/MS [M+H]⁺: 542.2.

Step-2: 1-(6-Chloro-7-ethyl-3,4-dihydro-2H-benzo[1,4]oxazine-2-carbonyl)-4-(4-fluoro-benzyl)-piperidine-4-carbonitrile

To a solution of 6-Chloro-2-[4-cyano-4-(4-fluoro-benzyl)-piperidine-1-carbonyl]-7-ethyl-2,3-dihydro-benzo[1,4]oxazine-4-carboxylic acid tert-butyl ester (180 mg, 0.33 mmol)) in DCM was added trifluoroacetic acid (1 ml). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration and preparative HPLC afforded 20 mg (13.7%) of 1-(6-Chloro-7-ethyl-3,4-dihydro-2H-benzo[1,4]oxazine-2-carbonyl)-4-(4-fluoro-benzyl)-piperidine-4-carbonitrile. LC/MS [M+H]⁺: 442.0. LCMS purity: 98.6%.

### Example-95

### 1-(6-Chloro-7-methoxy-3,4-dihydro-2H-benzo[1,4]oxazine-2-carbonyl)-4-[(4-fluoro-phenyl)-hydroxy-methyl]-piperidine-4-carbonitrile

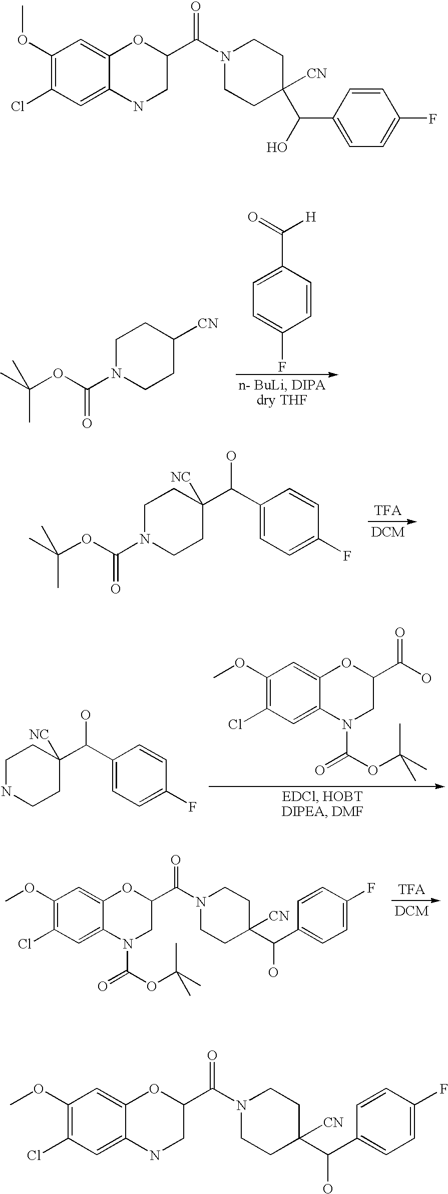

6-Chloro-7-methoxy-2,3-dihydro-benzo[1,4]oxazine-2,4-dicarboxylic acid-4-tert-butyl ester was prepared by a process as described in example 27.
Step-1: 4-Cyano-4-[(4-fluoro-phenyl)-hydroxy-methyl]-piperidine-1-carboxylic acid tent-butyl ester

To a solution of of DIPA (1.6 ml, 11.41 mmol) in dry THF, was added n-BuLi (13.31 mmol) solution dropwise at 0° C. The mixture was allowed to stir for 1 hr at room temperature. To this mixture a solution of N-boc-4-cyanopipyridine (2.0 g, 9.51 mmol) in dry THF was added dropwise at −78° C. The resulting mixture was allowed to stir for two hours at −78° C. to 0° C. Then a solution of 4-Fluoro benzaldehyde (1.1 ml, 9.51 mmol) in dry THF was added dropwise at −78° C. and the resulting mixture was allowed to stir at room temperature overnight. The reaction was quenched with saturated NH₄Cl solution in cold condition and stirred at room temperature for 30 minutes. THF layer was separated and the aqueous layer was extracted with ethyl acetate. The combined organic layers (THF and ethyl acetate) was washed with water and brine, then dried over Na₂SO₄ and concentrated under reduced pressure Purification of the crude by column chromatography (Si-gel, 20% ethyl acetate-hexane) afforded 1.2 g (38%) of 4-Cyano-4-[(4-fluoro-phenyl)-hydroxy-methyl]-piperidine-1-carboxylic acid tert-butyl ester. LC/MS [M+H]⁺: 335.2. ‘H-NMR (400 MHz, DMSO-d₆) δ (ppm): 7.43 (t, 2H, J=7.6 Hz, 5.72 Hz), 7.08 (t, 2H, J=8.32 Hz, 8.44 Hz), 4.51 (s, H), 4.14 (m, 2H), 2.95 (m, 2H), 2.29(s, H), 2.22 (d, H, J=13.44 Hz), 1.54(s, H), 1.49 (s, H), 1.43 (s, 9H).

Step-2: 4-[(4-Fluoro-phenyl)-hydroxy-methyl]-piperidine-4-carbonitrile

To a solution of 4-Cyano-4-[(4-fluoro-phenyl)-hydroxy-methyl]-piperidine-1-carboxylic acid tert-butyl ester (1.2 g, 3.59 mmol) in DCM was added trifluoro acetic acid (2.76 ml, 35.9 mmol) dropwise in cold condition. The mixture was allowed to stir at room temperature for one and half an hour. Then DCM was evaporated and the crude mass was diluted with water, neutralized with 2N NaOH solution and extracted with ethyl acetate. Ethyl acetate extract was washed with water and brine, and then dried over Na₂SO₄. Concentration of the organic extract under reduced pressure afforded 700 mg (83%) of 4-[(4-Fluoro-phenyl)-hydroxy-methyl]-piperidine-4-carbonitrile. LC/MS [M+H]⁺: 235.2

¹H-NMR (400 MHz, DMSO-d₆) δ (ppm): 7.45 (t, 2H, J=6.8 Hz, 6.68 Hz), 7.19 (t, 2H, J=8.8 Hz, 8.68 Hz), 6.09 (s, H), 4.47 (s, H), 2.93 (d, H, J=12.28 Hz), 2.83 (d, H, J=12.64 Hz), 2.61 (t, H, J=12.4 Hz, 12.6 Hz), 2.02 (m, H), 1.45 (m, H), 1.14 (d, H, J=12.4 Hz).

Step-3: 6-Chloro-2-{4-cyano-4-[(4-fluoro-phenyl)-hydroxy-methyl]-piperidine-1-carbonyl}-7-methoxy-2,3-dihydro-benzo[1,4]oxazine-4-carboxylic acid tert-butyl ester

To a solution of 6-Chloro-7-methoxy-2,3-dihydro-benzo[1,4]oxazine-2,4-dicarboxylic acid-4-tert-butyl ester (100 mg, 0.29 mmol) in DMF was added EDCI (72.5 mg, 0.38 mmol), HOBT (19.6 mg, 0.14 mmol) and DIPEA (0.15 ml 0.87 mmol) at 5° C. The resulting mixture was allowed to stir at room temperature for 30 minutes. Then a solution of 4-[(4-Fluoro-phenyl)-hydroxy-methyl]-piperidine-4-carbonitrile (68 mg, 0.29 mmol) in 0.3 ml DMF was added to it at 5° C. and the reaction mixture was allowed to stir at room temperature overnight. The reaction mass was diluted with ice-water, stirred for 10 minutes and extracted with ethyl acetate. Ethyl acetate layer was washed with water and brine, then dried over Na₂SO₄ and concentrated under reduced pressure. Purification of the crude by column chromatography (Si-gel, 0.75% MeOH-DCM) afforded 155 mg (96%) of 6-Chloro-2-{4-cyano-4-[(4-fluoro-phenyl)-hydroxy-methyl]-piperidine-1-carbonyl}-7-methoxy-2,3-dihydro-benzo[1,4]oxazine-4-carboxylic acid tert-butyl ester. LC/MS [M+H]⁺: 560.0. ¹H-NMR (400 MHz, DMSO-d₆) δ (ppm): 7.95 (s, H), 7.48 (m, 2H), 7.22 (t, 2H, J=8.64 Hz, 8.60 Hz), 6.69 (m, H), 6.24 (m, H), 5.47 (d, H, J=15.28), 5.29 (m, H), 4.59 (m, H), 4.43 (m, H), 4.17 (m, H), 3.79 (s, 3H), 2.89 (s, 2H), 2.73 (s, 2H), 2.22 (m, H), 1.73 (m, H), 1.46 (s, 9H).

Step-4: 1-(6-Chloro-7-methoxy-3,4-dihydro-2H-benzo[1,4]oxazine-2-carbonyl)-4-[(4-fluoro-phenyl)-hydroxy-methyl]-piperidine-4-carbonitrile

To a solution of 6-Chloro-2-{4-cyano-4-[(4-fluoro-phenyl)-hydroxy-methyl]-piperidine-1-carbonyl}-7-methoxy-2,3-dihydro-benzo[1,4]oxazine-4-carboxylic acid tert-butyl ester (130 mg, 0.23 mmol) in DCM was added trifluoro acetic acid (0.18 ml,2.32 mmol) dropwise in cold condition. The mixture was allowed to stir at room temperature 2 hours. Then DCM was evaporated and the crude mass was diluted with water and washed with 50% ether-hexane. The aqueous part was basified with 2N NaOH solution and extracted with ethyl acetate. Ethyl acetate extract was washed with water and brine, then dried over Na₂SO₄. Concentration of the organic extract under vacuum afforded 100 mg (93%) of 1-(6-Chloro-7-methoxy-3,4-dihydro-2H-benzo[1,4]oxazine-2-carbonyl)-4-[(4-fluoro-phenyl)-hydroxy-methyl]-piperidine-4-carbonitrile. LC/MS [M+H]⁺: 460.2. ¹H-NMR (400 MHz, DMSO-d₆) δ (ppm): 7.49 (s, H), 7.22 (t, 2H, J=8.72 Hz), 6.59 (m, 2H), 6.21 (m, H), 5.57 (s, H), 4.97 (m, H), 4.57(s, H), 4.38(m, H), 4.15 (m,H), 3.71 (s, 3H) 3.32 (m, H), 3.20 (m, H), 2.67 (m, 2H), 2.18 (m,H), 1.72 (m, H), 1.54 (m, H)1.29 (m,H). HPLC: 91.6%.

### Example 96

### 1-(6-Chloro-7-dimethylamino-3,4-dihydro-2H-benzo[1,4]oxazine-2-carbonyl)-3-(4-fluoro-benzyl)-pyrrolidine-3-carbonitrile

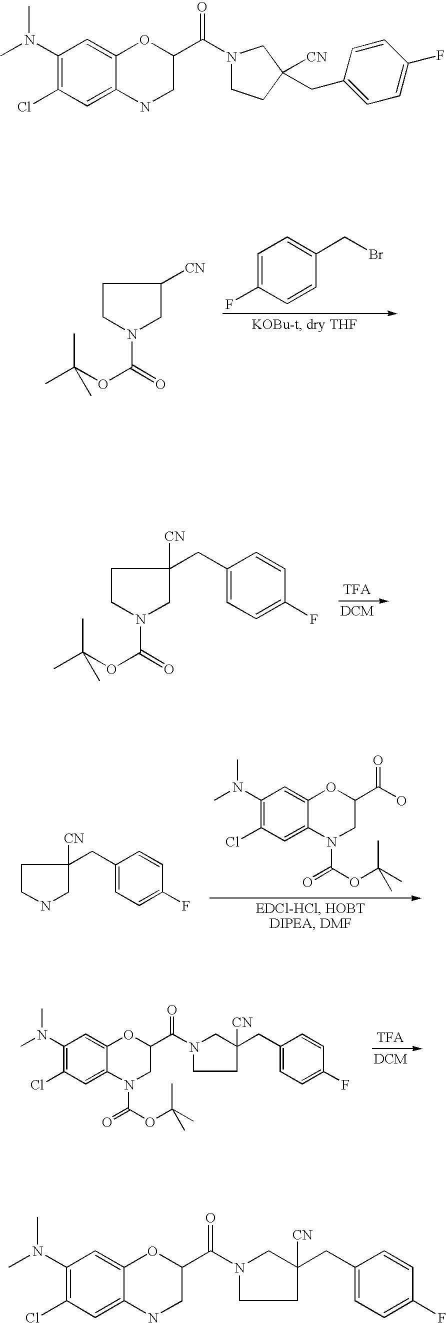

6-Chloro-7-dimethylamino-2,3-dihydro-benzo[1,4]oxazine-2,4-dicarboxylic acid 4-tert-butyl ester was prepared by a process as described in example 51.
Step 1: 3-Cyano-3-(4-fluoro-benzyl)-pyrrolidine-1-carboxylic acid tert-butyl ester

Potassium tert. butoxide (137 mg, 1.22 mmol) was added to a cold stirring solution of 3-Cyano-pyrrolidine-1-carboxylic acid tert-butyl ester (200 mg, 1.02 mmol) in THF and the resulting mixture was stirred for an hour. Then 1-Bromomethyl-4-fluoro-benzene (0.13 ml, 1.02 ml, 1 eqv) was added dropwise at 0° C. The temperature of reaction mixture was allowed to rise and the mixture was stirred at room temperature overnight. Then the reaction mass was quenched with ice-water and was stirred for 10 minutes. THF layer was separated and the aqueous part was extracted with ethyl acetate. Combined organic layers were washed with water and brine, dried over Na₂SO₄ and concentrated. Purification of the crude by column chromatography (Si-gel, 10% ethyl acetate in hexane) afforded 65 mg of 3-Cyano-3-(4-fluoro-benzyl)-pyrrolidine-1-carboxylic acid tert-butyl ester. LC/MS [M+H]⁺: 305.0. ¹H-NMR (400 MHz, DMSO-d₆) δ (ppm): 7.35 (m, 2H), 7.20 (m, 2H), 3.56 (m, 1H), 3.47 (m, 1H), 3.31 (m, 2H), 3.02 (m, 2H), 2.13 (bs, 2H), 1.40 (s, 9H).

Step 2: 3-(4-Fluoro-benzyl)-pyrrolidine-3-carbonitrile

To a solution of 3-Cyano-3-(4-fluoro-benzyl)-pyrrolidine-1-carboxylic acid tert-butyl ester (65 mg) in 5 ml of DCM was added trifluoro acetic acid (0.3 ml) dropwise in cold condition. The mixture was allowed to stir at room temperature for 2 hours. Then DCM was evaporated and the crude mass was diluted with water and basified with 2N NaOH solution and extracted with ethyl acetate. Ethyl acetate extract was washed with water and brine, dried over Na₂SO₄ and concentrated under reduced pressure to afford 35 mg of 3-(4-Fluoro-benzyl)-pyrrolidine-3-carbonitrile. LC/MS [M+H]⁺: 205.3.

Step3: 6-Chloro-2-[3-cyano-3-(4-fluoro-benzyl)-pyrrolidine-1-carbonyl]-7-dimethylamino-2,3 dihydro-benzo[1,4]oxazine-4-carboxylic acid tert-butyl ester

To a solution of 6-Chloro-7-dimethylamino-2,3-dihydro-benzo[1,4]oxazine-2,4-dicarboxylic acid 4-tert-butyl ester (50 mg, 0.14 mmol) in DMF was added EDCI (35 mg, 0.18 mmol), HOBT (9.5 mg, 0.07 mmol) and DIPEA (0.07 ml 0.42 mmol) at 5° C. The resulting mixture was allowed to stir at room temperature for 30 minutes and was added a solution of 3-(4-Fluoro-benzyl)-pyrrolidine-3-carbonitrile (28.6 mg, 0.14 mmol) in DMF. The reaction mixture was allowed to stir at room temperature overnight. The reaction mass was diluted with ice-water, stirred for 10 minutes and extracted with ethyl acetate. Ethyl acetate layer was washed with water and brine, then dried over Na₂SO₄ and concentrated under reduced pressure. Purification of the crude by column chromatography (Si-gel, 1% MeOH—DCM) afforded 32 mg (42%) of 6-Chloro-2-[3-cyano-3-(4-fluoro-benzyl)-pyrrolidine-1-carbonyl]-7-dimethylamino-2,3 dihydro-benzo[1,4]oxazine-4-carboxylic acid tert-butyl ester. LC/MS [M+H]⁺: 543.4.

Step-4: 1-(6-Chloro-7-dimethylamino-3,4-dihydro-2H-benzo[1,4]oxazine-2-carbonyl)-3-(4-fluoro-benzyl)-pyrrolidine-3-carbonitrile

To a solution of 6-Chloro-2-[3-cyano-3-(4-fluoro-benzyl)-pyrrolidine-1-carbonyl]-7-dimethylamino-2,3 dihydro-benzo[1,4]oxazine-4-carboxylic acid tert-butyl ester (30 mg, 0.05 mmol) in DCM was added trifluoro acetic acid (0.04 ml, 0.55 mmol) dropwise in cold condition. The mixture was allowed to stir at room temperature for 2 hours. Then DCM was evaporated and the crude mass was diluted with water and basified with 2N NaOH solution and extracted with ethyl acetate. The organic layer was washed with water and brine, dried over Na₂SO₄ and concentrated under reduced pressure. Purification of the crude by column chromatography (Si-gel, 1% MeOH—DCM) afforded 18 mg (75%) of 1-(6-Chloro-7-dimethylamino-3,4-dihydro-2H-benzo[1,4]oxazine-2-carbonyl)-3-(4-fluoro-benzyl)-pyrrolidine-3-carbonitrile. LC/MS [M+H]⁺: 443.1. ¹H-NMR (400 MHz, DMSO-d₆) δ (ppm): 7.36 (m, 2H), 7.20 (m, 2H), 6.61 (s, 2H), 5.77 (m, 1H), 4.79 (bs, 1H), 4.11 (m, 1H), 3.85 (m, 1H), 3.68 (m, 2H), 3.47 (m, 2H), 3.06 (m, 2H), 2.58 (s, 6H), 2.27 (m, 2H). HPLC: 96.2%.

### Example 97

### {6-Chloro-2-[4-cyano-4-(4-fluoro-benzyl)-piperidine-1-carbonyl]-3,4-dihydro-2H-benzo[1,4]oxazin-7-yl-methyl-cyanamide

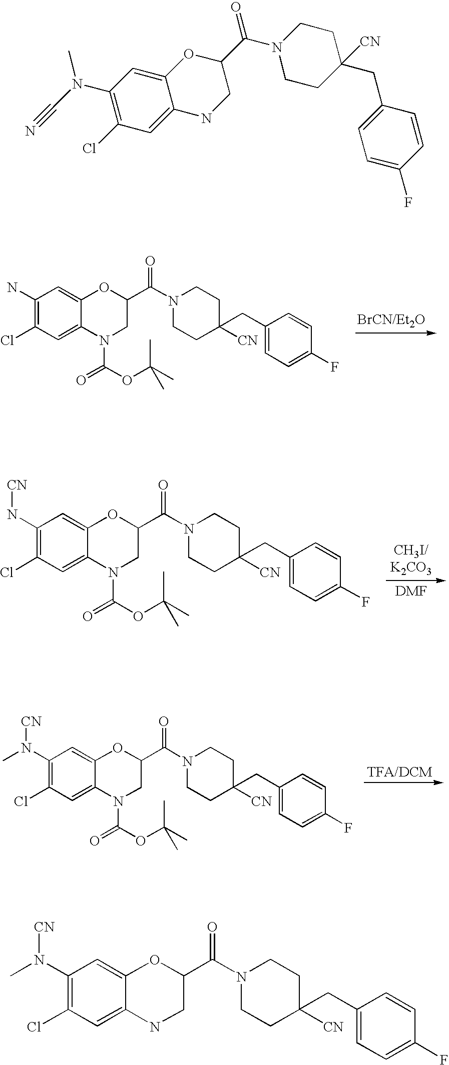

7-Amino-6-chloro-2-[((5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester was prepared as described in example 51.
Step-1: 6-Chloro-7-cyanoamino-2-[((5Z,7E)-3-cyano-3-ethyl-8-fluoro-5-vinyl-nona-5,7-dienyl)- methyl-carbamoyl]-2,3-dihydro-benzo[1,4]oxazine-4-carboxylic acid tert-butyl ester

To a solution of 7-Amino-6-chloro-2-[((5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester (100 mg, 0.18 mmol) in diethyl ether was added cyanogen bromide (100 mg, 0.94 mmol). The reaction mixture was stirred at room temperature for 2 days. The precipitated solid was filtered off and mother liquor was concentrated. The organic crude was purified over column chromatography (Si-gel, 30% ethyl acetate in hexane) to afford 25 mg (23.7%) of 6-Chloro-7-cyanoamino-2-[((5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester. LC/MS [M+H]⁺:554.4.

Step-2: 6-Chloro-2-[((5Z7E)-3-cyano-3-ethyl-8-fluoro-5-vinyl-nona-5,7-dienyl)-methyl-carbamoyl]-7-(cyano-methyl-amino)-2,3-dihydro-benzo[1,4]oxazine-4-carboxylic acid tert-butyl ester

To a solution of 6-Chloro-7-cyanoamino-2-[((5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester (25 mg, 0.04 mmol) in DMF was added anhd. K₂CO₃ (12 mg, 0.09 mmol) and CH₃I (0.001 ml, 0.09 mmol). The reaction mixture was heated at 40° C. for 3 h. The reaction mixture was diluted with water and extracted with ethyl acetate, washed the organic layer with water, brine and dried over Na₂SO₄. Concentration of organic layer under reduced pressure afforded 25 mg (100%) of (6-Chloro-2-[((5Z,7E)-3-cyano-3-ethyl-8-fluoro-5-vinyl-nona-5,7-dienyl)-methyl-carbamoyl]-7-(cyano-methyl-amino)-2,3-dihydro-benzo[1,4]oxazine-4-carboxylic acid tert-butyl ester. LC/MS [M+H]⁺: 568.6.

Step-3: 6-Chloro-7-(cyano-methyl-amino)-3,4-dihydro-2H-benzo[1,4]oxazine-2-carboxylic acid ((5Z,7E)-3-cyano-3-ethyl-8-fluoro-5-vinyl-nona-5,7-dienyl)-methyl-amide

To a solution of 6-Chloro-2-[((5Z,7E)-3-cyano-3-ethyl-8-fluoro-5-vinyl-nona-5,7-dienyl)-methyl-carbamoyl]-7-(cyano-methyl-amino)-2,3-dihydro-benzo[1,4]oxazine-4-carboxylic acid tert-butyl ester. (25 mg, 0.04 mmol) in DCM was added trifluoroacetic acid (0.3 ml, 3.9 mmol). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 6 mg (30%) of 6-Chloro-7-(cyano-methyl-amino)-3,4-dihydro-2H-benzo[1,4]oxazine-2-carboxylic acid ((5Z,7E)-3-cyano-3-ethyl-8-fluoro-5-vinyl-nona-5,7-dienyl)-methyl-amide. LC/MS [M+H]⁺: 468.4.

### Example 98

### 1-(6-Chloro-7-methoxy-3,4-dihydro-2H-benzo[1,4]oxazine-2-carbonyl)-4-[difluoro-(4-fluoro-phenyl)-methyl]-piperidine-4-carbonitrile

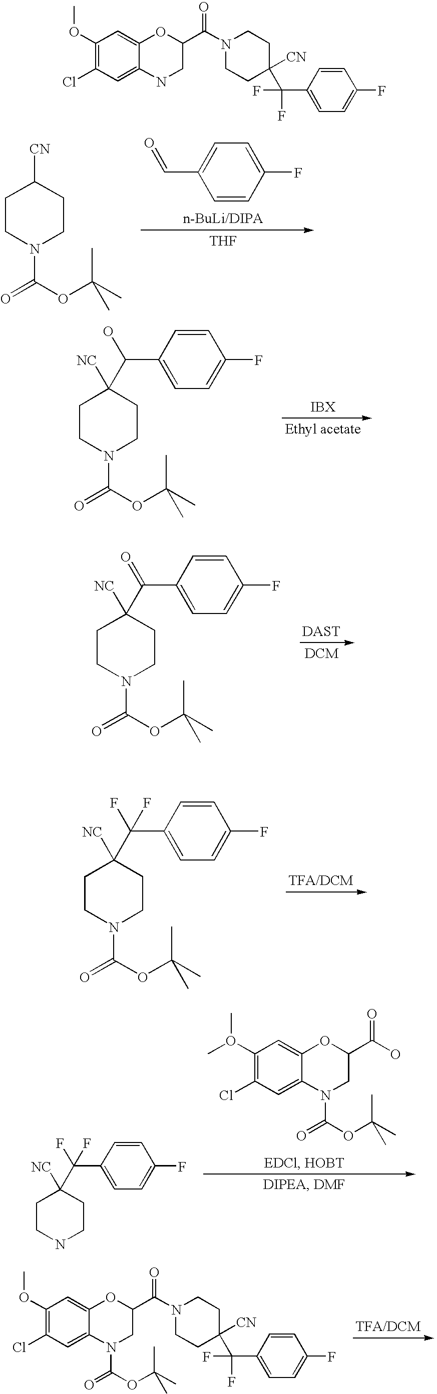

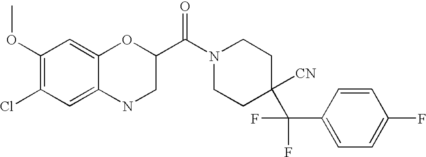

Step-1: 4-Cyano-4-[(4-fluoro-phenyl)-hydroxy-methyl]-piperidine-1-carboxylic acid tert-butyl ester

To a stirred solution of DIPA (0.8 ml, 5.71 m.mol) in THF, n-BuLi (1.57M) (4.24 ml, 6.66 m.moll) was added to it drop wise in ice-cold condition. The reaction mixture was stirred for 1 hour and was added 4-Cyano-piperidine-1-carboxylic acid tert-butyl ester (1 g, 4.76 m.mol) at −78° C. The reaction mixture was stirred for another 2 hours at room temperature and added 4-Fluoro-benzaldehyde (0.54 ml, 4.76 m.mol,) and stirred the reaction mixture for 3 h at room temperature, quenched the reaction mixture with saturated NH₄Cl solution and extracted with ethyl acetate. The organic layer was washed with water, brine, dried over Na₂SO₄ and concentrated the solvent under reduced pressure. Thus obtained crude was purified over column chromatography (Si-gel, 12% ethyl acetate-hexane) to afford 1.0 g (52.5%) of 4-Cyano-4-[(4-fluoro-phenyl)-hydroxy-methyl]-piperidine-1-carboxylic acid tert-butyl ester. ¹H-NMR (400 MHz, CDCl₃) δ (ppm): 7.43(m, 2H), 7.1(m, 2H), 4.51(d, 1H), 4.12(m, 3H)), 2.99(m, 3H), 2.32(d, 1H), 2.25(d, 2H), 1.44(s, 9H). LC/MS [M+H]⁺: 335.3.

Step-2: 4-Cyano-4-(4-fluoro-benzoyl)-piperidine-1-carboxylic acid tert-butyl ester

To a stirred solution of 4-Cyano-4-[(4-fluoro-phenyl)-hydroxy-methyl]-piperidine-1-carboxylic acid tert-butyl ester (200 mg, 0.6 mmol) in ethyl acetate, IBX (335 mg, 1.2 mmol) was added. The reaction mixture was refluxed for 5 h and filtered through celite bed. The solution was concentrated under reduced pressure to afford 180 mg (92.3%) of 4-Cyano-4-(4-fluoro-benzoyl)-piperidine-1-carboxylic acid tert-butyl ester. ¹H-NMR (400 MHz, CDCl₃) δ (ppm): 8.2 (m, 2H), 7.18 (m, 2H), 4.19 (m, 2H), 3.23 (m, 2H), 2.17-1.2 (m, 4H), 1.46 (s, 9H). LC/MS [M+H]⁺: 233.4.

Step-3: 4-Cyano-4-[difluoro-(4-fluoro-phenyl)-methyl]-piperidine-1-carboxylic acid tert-butyl ester

To a solution of 4-Cyano-4-(4-fluoro-benzoyl)-piperidine-1-carboxylic acid tert-butyl ester (180 mg, 0.54 mmol) in DCM, DAST (2.2 ml, 18.4 mmol) was added. The reaction mixture was stirred for 2 h at room temperature. The reaction mixture was diluted with water and organic layer was separated. The organic layer was washed with water and concentrated under reduced pressure to afford 190 mg (99.3%) of 4-Cyano-4-[difluoro-(4-fluoro-phenyl)-methyl]-piperidine-1-carboxylic acid tert-butyl ester. GCMS (m/z):354.

Step-4: 4-[Difluoro-(4-fluoro-phenyl)-methyl]-piperidine-4-carbonitrile

To a solution of 4-Cyano-4-[difluoro-(4-fluoro-phenyl)-methyl]-piperidine-1-carboxylic acid tert-butyl ester (190 mg, 0.54 mmol) in DCM was added trifluoroacetic acid (0.41 ml, 5.4 mmol). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 130 mg (94.7%) of 4-[Difluoro-(4-fluoro-phenyl)-methyl]-piperidine-4-carbonitrile. ¹H-NMR (400 MHz, CDCl₃) δ (ppm): 7.53(m, 2H), 7.17(m, 2H), 3.15(d, 2H), 2.9(t, 2H), 1.89-1.77(m, 4H), 1.25(s, 1H). GCMS(m/z): 254.

Step-5: 6-Chloro-2-{4-cyano-4-[difluoro-(4-fluoro-phenyl)-methyl]-piperidine-1-carbonyl}-7-methoxy-2,3-dihydro-benzo[1,4]oxazine-4-carboxylic acid tert-butyl ester

To a stirred solution of 6-Chloro-7-methoxy-2,3-dihydro-benzo[1,4]oxazine-2,4-dicarboxylic acid 4-tert-butyl ester (81 mg, 0.236 mmol) in DMF was added DIPEA (0.12 ml, 0.71 mmol), EDCI (90 mg, 0.47 mmol) and HOBT (48 mg, 0.35 mmol) at 0° C. The reaction mixture was stirred for 1 h at room temperature and was added 4-[Difluoro-(4-fluoro-phenyl)-methyl]-piperidine-4-carbonitrile (60 mg, 0.24 mmol). The reaction mixture was stirred for overnightand diluted with water. The precipitated solid was filtered and dried to afford 130 mg (95.8%) of 6-Chloro-2-{4-cyano-4-[difluoro-(4-fluoro-phenyl)-methyl]-piperidine-1-carbonyl}-7-methoxy-2,3-dihydro-benzo[1,4]oxazine-4-carboxylic acid tert-butyl ester. ¹H-NMR (400 MHz, CDCl₃) δ (ppm): 7.56-6.46(m, 6H), 4.87-4.17(m, 4H), 3.83(s, 3H), 3.72-3.38(m, 3H), 2.86(m, 1H), 2.03-1.82(m, 3H), 1.54(s, 9H). LC/MS [M+H]⁺: 580.4.

Step-6: 1-(6-Chloro-7-methoxy-3,4-dihydro-2H-benzo[1,4]oxazine-2-carbonyl)-4-[difluoro-(4-fluoro-phenyl)-methyl]-piperidine-4-carbonitrile

To a stirred solution of 6-Chloro-2-{4-cyano-4-[difluoro-(4-fluoro-phenyl)-methyl]-piperidine-1-carbonyl}-7-methoxy-2,3-dihydro-benzo[1,4]oxazine-4-carboxylic acid tert-butyl ester (130 mg, 0.23 mmol) in DCM, TFA (0.17 ml, 2.25 mmol) was added in ice-cold condition. The reaction mixture was stireed for 2 h at room temperature and, concentrared the reaction mixture, diluted with water, basified the compound with NaHCO₃ solution and extracted with ethyl acetate. The organic layer was wased with water, brine, dried over Na₂SO₄ and concentrated under reduced pressure to afford 50 mg (45.3%) of 1-(6-Chloro-7-methoxy-3,4-dihydro-2H-benzo[1,4]oxazine-2-carbonyl)-4-[difluoro-(4-fluoro-phenyl)-methyl]-piperidine-4-carbonitrile. LC/MS [M+H]⁺: 480.2. ¹H-NMR (400 MHz, DMSO-d₆) δ (ppm): 7.64-6.55(m, 6H), 5.58(s, 1H), 5.0-4.19(m, 3H), 3.7(s, 3H), 3.21(m, 2H), 1.99-1.34(m, 6H). HPLC: 97.6%

### Example 99

### 1-(6-Chloro-7-dimethylamino-3,4-dihydro-2H-benzo[1,4]oxazine-2-carbonyl)-4-[difluoro-(4-fluoro-phenyl)-methyl]-piperidine-4-carbonitrile

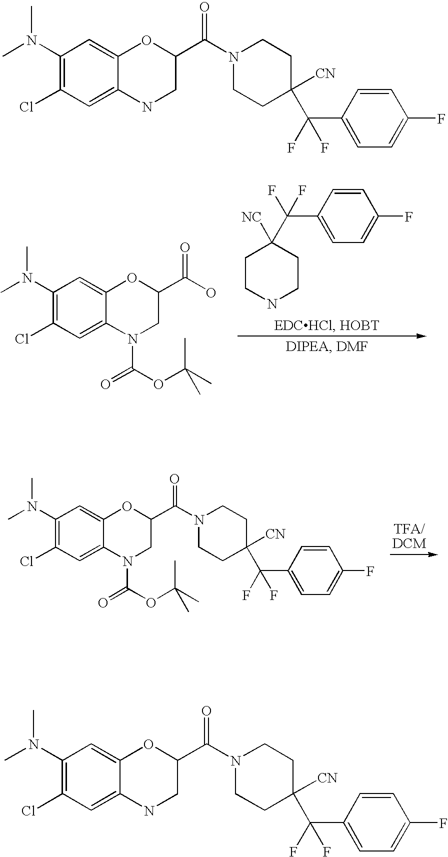

4-[Difluoro-(4-fluoro-phenyl)-methyl]-piperidine-4-carbonitrile was prepared by a process as described in example 98.
Step-1: 6-Chloro-2-{4-cyano-4-[difluoro-(4-fluoro-phenyl)-methyl]-piperidine-1-carbonyl}-7-dimethylamino-2,3-dihydro-benzo[1,4]oxazine-4-carboxylic acid tert-butyl ester

To a stirred solution of 6-Chloro-7-dimethylamino-2,3-dihydro-benzo[1,4]oxazine-2,4-dicarboxylic acid 4-tert-butyl ester (180 mg, 0.236 m.mol) in DMF was added DIPEA (0.12 ml, 0.708 m.mol), EDCI (90 mg, 0.472 m.mol) and HOBT (48 mg, 0.354 m.mol) were added at 0° C. The reaction mixture was stirred for lh at room temperature and added 4-[Difluoro-(4-fluoro-phenyl)-methyl]-piperidine-4-carbonitrile (60 mg, 0.236 m.mol. The reaction mixture was stirred for overnight and diluted with water. The precipitated solid was filtered and dried to afford 110 mg (79.2%) of 6-Chloro-2-{4-cyano-4-[difluoro-(4-fluoro-phenyl)-methyl]-piperidine-1-carbonyl}-7-dimethylamino-2,3-dihydro-benzo[1,4]oxazine-4-carboxylic acid tert-butyl ester. LC/MS [M+H]⁺: 593.4. ¹H-NMR (400 MHz, CDCl₃) δ (ppm): 7.54-6.56 (m, 6H), 4.84-2.84 (m, 6H), 2.75 (s, 6H), 2.34-1.82 (m, 5H), 1.52 (s, 9H).

Step-2: (6-Chloro-7-dimethylamino-3,4-dihydro-2H-benzo[1,4]oxazin-2-yl)-{4-[difluoro-(4-fluoro-phenyl)-methyl]-piperidin-1-yl}-methanone

To a solution of 6-Chloro-2-{4-cyano-4-[difluoro-(4-fluoro-phenyl)-methyl]-piperidine-1-carbonyl}-7-dimethylamino-2,3-dihydro-benzo[1,4]oxazine-4-carboxylic acid tert-butyl ester (110 mg, 0.19 mmol) in DCM was added trifluoroacetic acid (0.14 ml, 1.86 mmol). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 35 mg (37.4%) of (6-Chloro-7-dimethylamino-3,4-dihydro-2H-benzo[1,4]oxazin-2-yl)-{4-[difluoro-(4-fluoro-phenyl)-methyl]-piperidin-1-yl}-methanone. LC/MS [M+H]⁺:493.2. ¹H-NMR (400 MHz, DMSO-d₆) δ (ppm): 7.63-6.54 (m, 6H), 5.74 (s, 1H), 5.0-4.2 (m, 3H), 3.2 (m, 2H), 2.67 (s, 1H), 2.56-2.5 (d, 6H), 1.99-1.34 (m, 5H). HPLC-93.02%.

### Example 100

### 1-[6-Chloro-7-(1H-pyrazol-4-yl)-3,4-dihydro-2H-benzo[1,4]oxazine-2-carbonyl]-4-(4-fluoro-benzyl)-piperidine-4-carbonitrile

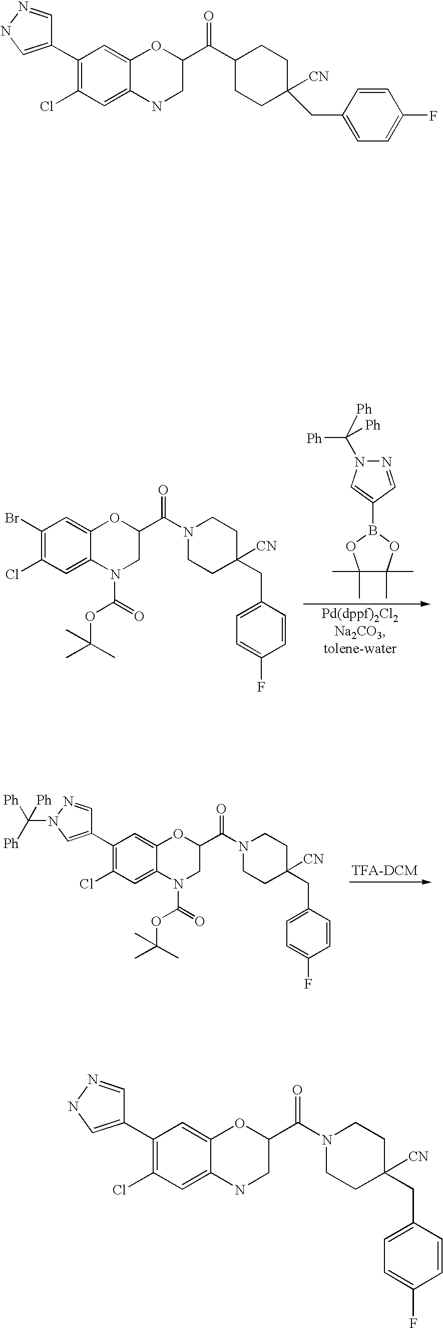

1-(7-Bromo-6-chloro-3,4-dihydro-2H-benzo[1,4]oxazine-2-carbonyl)-4-(4-fluoro-benzyl)-piperidine-4-carbonitrile was prepared by a process described in example 57.
Step-1: (6-Chloro-2-[4-cyano-4-(4-fluoro-benzyl)-piperidine-1-carbonyl]-7-(1-trityl-1H-pyrazol-4-yl)-2,3-dihydro-benzo[1,4]oxazine-4-carboxylic acid tert-butyl ester)

A solution of 7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester (100 mg, 0.1687 mmol) and 4-(4,4,5,5-Tetramethyl-[1,3,2]dioxaborolan-2-yl)-1-trityl-1H-pyrazole in tolune-water (1:1) and was degassed for one hour. To that solution were added sodium carbonate (36 mg, 0.3374 mmol) and catalytic amount of Pd(dppf)₂Cl₂ (10 mole %). The reaction mixture was refluxed for 12 hours and diluted with water. The organics was extracted with ethyl acetate, dried over sodium sulfate and concentrated under reduced pressure to get the crude mass. Purification of that crude mass by flash chromatography (Si-gel, 15% ethyl acetate-hexane) afforded 105 mg (81%) of (6-Chloro-2-[4-cyano-4-(4-fluoro-benzyl)-piperidine-1-carbonyl]-7-(1-trityl-1H-pyrazol-4-yl)-2,3-dihydro-benzo[1,4]oxazine-4-carboxylic acid tert-butyl ester). ¹H-NMR (400 MHz, DMSO-d₆) δ (ppm): 8.10 (s, 1H), 8.04 (s, 1H), 7.58-7.10 (m, 21H), 5.43 (s, 1H), 4.33 (m, 1H), 4.11 (bs, 2H), 3.65 (m, 1H), 3.14 (m, 2H), 2.92 (m, 2H), 2.50 (m, 2H), 1.82 (m, 2H), 1.46 (m, 9H).

Step-2: (1-[6-Chloro-7-(1H-pyrazol-4-yl)-3,4-dihydro2H-benzo[1,4]oxazine-2-carbonyl]-4-(4-fluoro-benzyl)-piperidine-4-carbonitrile)

To a solution of 6-Chloro-2-[4-cyano-4-(4-fluoro-benzyl)-piperidine-1-carbonyl]-7-(1-trityl-1H-pyrazol-4-yl)-2,3-dihydro-benzo[1,4]oxazine-4-carboxylic acid tert-butyl ester (100 mg, 0.12 mmol) was added trifluoro acetic acid (0.09 ml, 1.22 mmol) and the reaction mixture was stirred for 16 hours. The reaction mixture was concentrated under reduced pressure and diluted with water, neutralized with 1(N) NaOH solution and extracted with ethyl acetate. The organic layer was dried over sodium sulfate and concentrated to afford 25 mg (42%) of (1-[6-Chloro-7-(1H-pyrazol-4-yl)-3,4-dihydro2H-benzo[1,4]oxazine-2-carbonyl]-4-(4-fluoro-benzyl)-piperidine-4-carbonitrile). LC/MS [M+H]⁺: 480.4. ¹H-NMR (400 MHz, DMSO-d₆) δ (ppm): 12.85 (bs, 1H), 7.85 (bs, 2H), 7.33 (bs, 2H), 7.18 (m, 2H), 6.92 (m, 1H), 6.67 (s, 1H), 6.15 (s, 1H), 4.97 (s, 1H), 4.40 (m, 1H), 4.11 (m, 1H), 3.40 (m, 2H), 3.10 (m, 2H), 3.07 (s, 1H), 2.68 (m, 1H), 1.81 (m, 2H), 1.54(m, 1H). HPLC: 98.8%.

### Example 101

### 6-Chloro-7-[methyl-(2H-tetrazol-5-yl)-amino]-3,4-dihydro-2H-benzo[1,4]oxazine-2-carboxylic acid [3-cyano-3-(4-fluoro-benzyl)-pentyl]-methyl-amide

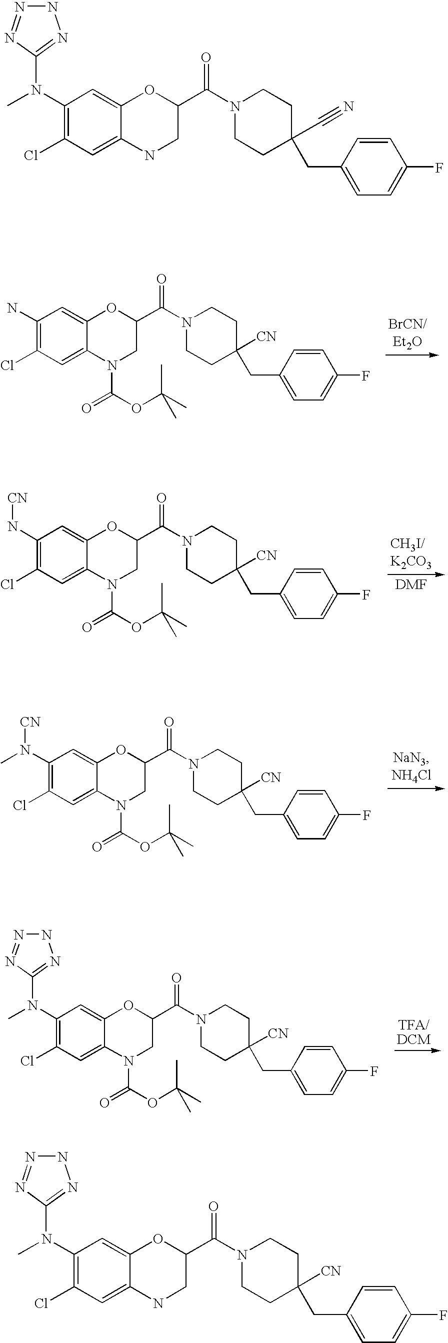

Step-1: 6-Chloro-7-cyanoamino-2-[((5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester

7-Amino-6-chloro-2-R(5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester (100 mg, 0.18 mmol) was taken in diethyl ether, added cyanogen bromide (100 mg, 0.94 mmol) and stirred at room temperature for two days. The reaction mixture was filtered, concentrated and purified over flash chromatography (Si-gel, 30% ethyl acetate-hexane) to afford 25 mg (22.2%) of 6-Chloro-7-cyanoamino-2-[((5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester. LC/MS [M+H]⁺:554.4.

Step-2 : 6-Chloro-2-[((5Z,7E)-3-cyano-3-ethyl-8-fluoro-5-vinyl-nona-5,7-dienyl)-methyl-carbamoyl]-7-(cyano-methyl-amino)-2,3-dihydro-benzo[1,4]oxazine-4-carboxylic acid tert-butyl ester

To a solution of 6-Chloro-7-cyanoamino-2-[((5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester (25 mg, 0.04 mmol) in DMF was added anhd. K₂CO₃(12 mg, 0.09 mmol) and CH₃I (13 mg. 0.0005 ml, 0.09 mmol). The reaction mixture was heated at 40° C. for 3 h., diluted with ice-water, extracted with ethyl acetate, washed with waterand concentrated to afford 25 mg (100%) of 6-Chloro-2-[((5Z,7E)-3-cyano-3-ethyl-8-fluoro-5-vinyl-nona-5,7-dienyl)-methyl-carbamoyl]-7-(cyano-methyl-amino)-2,3-dihydro-benzo[1,4]oxazine-4-carboxylic acid tert-butyl ester. LC/MS [M+H]⁺: 568.6

Step-3: 6-Chloro-2-[((5Z,7E)-3-cyano-3-ethyl-8-fluoro-5-vinyl-nona-5,7-dienyl)-methyl-carbamoyl]-7-[methyl-(2H-tetrazol-5-yl)-amino]-2,3-dihydro-benzo[1,4]oxazine-4-carboxylic acid tert-butyl ester

To a solution of 6-Chloro-2-[((5Z,7E)-3-cyano-3-ethyl-8-fluoro-5-vinyl-nona-5,7-dienyl)-methyl-carbamoyl]-7-(cyano-methyl-amino)-2,3-dihydro-benzo[1,4]oxazine-4-carboxylic acid tert-butyl ester (190 mg, 0.34 mmol) in DMF was added sodium azide (87 mg, 1.3 mmol) and ammonium chloride (72 mg, 1.3 mmol). The reaction mixture was heated at 100° C. overnight. The reaction mixture was diluted with water and precipitated solid was filtered. The solid was washed with water and dried to afford 110 mg (50.3%) of 6-Chloro-2-[((5Z,7E)-3-cyano-3-ethyl-8-fluoro-5-vinyl-nona-5,7-dienyl)-methyl-carbamoyl]-7-[methyl-(2H-tetrazol-5-yl)-amino]-2,3-dihydro-benzo[1,4]oxazine-4-carboxylic acid tert-butyl ester. LC/MS [M+H]⁺: 611.2

Step-4: 6-Chloro-7-[methyl-(2H-tetrazol-5-yl)-amino]-3,4-dihydro-2H-benzo[1,4]oxazine-2-carboxylic acid ((5Z,7E)-3-cyano-3-ethyl-8-fluoro-5-vinyl-nona-5,7-dienyl)-methyl-amide

To a solution of 6-Chloro-2-[((5Z,7E)-3-cyano-3-ethyl-8-fluoro-5-vinyl-nona-5,7-dienyl)-methyl-carbamoyl]-7-[methyl-(2H-tetrazol-5-yl)-amino]-2,3-dihydro-benzo[1,4]oxazine-4-carboxylic acid tert-butyl ester (110 mg, 0.18 mmol)) in DCM was added trifluoroacetic acid (1.14 ml, 1.8 mmol). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 20 mg (20.4%) of (6-Chloro-7-[methyl-(2H-tetrazol-5-yl)-amino]-3,4-dihydro-2H-benzo[1,4]oxazine-2-carboxylic acid ((5Z,7E)-3-cyano-3-ethyl-8-fluoro-5-vinyl-nona-5,7-dienyl)-methyl-amide. LC/MS [M+H]⁺: 511.4. ¹H-NMR (400 MHz, DMSO-d₆) δ (ppm): 7.35-6.68 (m, 6 H), 6.34 (bs, 1H), 5.80 (m, 1H), 4.94 (m, 2H), 4.40 (m, 1H), 4.10 (m, 1H), 3.41-1.72 (m, 12 H), HPLC purity: 89.6%.

### Example 102

### 1-[6-Chloro-7-(2H-tetrazol-5-ylamino)-3,4-dihydro-2H-benzo[1,4]oxazine-2-carbonyl]-4-(4-fluoro-benzyl)-piperidine-4-carbonitrile

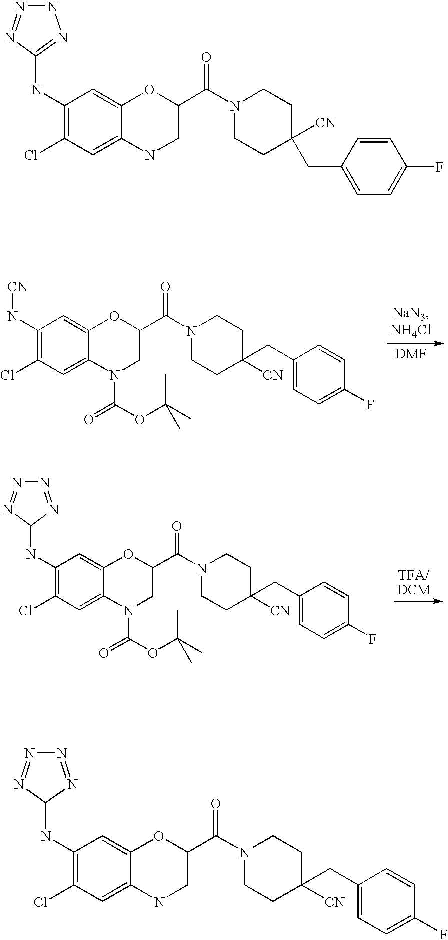

6-Chloro-7-cyanoamino-2-[((5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester was prepared by a process as described in example 101.
Step-1: 6-Chloro-2-N5Z7E)-3-cyano-3-ethyl-8-fluoro-5-vinyl-nona-5,7-dienyl)-methyl-carbamoyl]-7-(5H-tetrazol-5-ylamino)-2,3-dihydro-benzo[1,4]oxazine-4-carboxylic acid tert-butyl ester

To a solution of 6-Chloro-7-cyanoamino-2-R(5Z,7E)-3-cyano-3-ethyl-8-fluoro-5-vinyl-nona-5,7-dienyl)-methyl-carbamoyl]-2,3-dihydro-benzo[1,4]oxazine-4-carboxylic acid tert-butyl ester (150 mg, 0.27 mmol) in DMF was added sodium azide (70 mg, 1.1 mmol) and ammonium chloride (58 mg, 1.08 mmol). The reaction mixture was heated at 100° C. overnight. The reaction mixture was diluted with water and precipitated solid was filtered. The solid was washed with water and dried to afford 70 mg (44.4%) of 6-Chloro-2-[((5Z,7E)-3-cyano-3-ethyl-8-fluoro-5-vinyl-nona-5,7-dienyl)-methyl-carbamoyl]-7-(5H-tetrazol-5-ylamino)-2,3-dihydro-benzo[1,4]oxazine-4-carboxylic acid tert-butyl ester. LC/MS [M+H]⁺: 597.4.

Step-2: 1-[6-Chloro-7-(2H-tetrazol-5-ylamino)-3,4-dihydro-2H-benzo[1,4]oxazine-2-carbonyl]-4-(4-fluoro-benzyl)-piperidine-4-carbonitrile

To a stirred solution of 6-Chloro-2-R(5Z,7E)-3-cyano-3-ethyl-8-fluoro-5-vinyl-nona-5,7-dienyl)-methyl-carbamoyl]-7-(5H-tetrazol-5-ylamino)-2,3-dihydro-benzo[1,4]oxazine-4-carboxylic acid tert-butyl ester (70 mg, 0.12 mmol) in DCM was added TFA (1.1 ml, 14.3 mmol) at 0° C. and stirred at room temperature for 2 h. The reaction mixture was concentrated and diluted with water, basified with NaOH solution, extracted with ethyl acetate. The organic layer was concentrated and purified through flash chromatography (Si-gel, 1% MeOH-DCM) to afford 10 mg (16.8%). LC/MS [M+H]⁺: 497.4.HPLC: 72.3%.

### Example 103

### 1-[6-Chloro-7-dimethylamino-4-(2,2,2-trifluoro-acetyl)-3,4-dihydro-2H-benzo[1,4]oxazine-2-carbonyl]-4-(4-fluoro-benzyl)-piperidine-4-carbonitrile

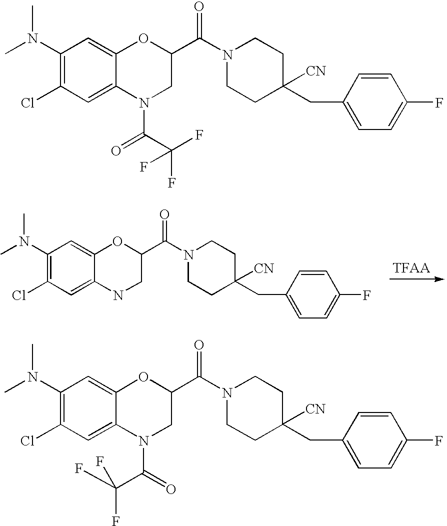

1-(6-Chloro-7-dimethylamino-3,4-dihydro-2H-benzo[1,4]oxazine-2-carbonyl)-4-(4-fluoro-benzyl)-piperidine-4-carbonitrile was prepared by a process described in example 51.

To a solution of 1-(6-Chloro-7-dimethylamino-3,4-dihydro-2H-benzo[1,4]oxazine-2-carbonyl)-4-(4-fluoro-benzyl)-piperidine-4-carbonitrile (100 mg, 0.438 mmol) in DCM was added tri ethyl amine (0.09 ml, 0.657 mmol) and tri fluoro acetic acid anhydride (0.07 ml, 0.5694) drop wise in ice cold condition. The reaction mixture was stirred for 3 hour at room temperature. The reaction mixture was diluted with water and stirred for half of an hour. Then the organic layer was separated, washed with brine, dried over sodium sulfate and concentrated under reduced pressure to get the crude mass which was then purified over column chromatography (Si-gel, 10% ethyl acetate in hexane) to afford 35 mg (14%) of 1-[6-Chloro-7-dimethylamino-4-(2,2,2-trifluoro-acetyl)-3,4-dihydro-2H-benzo[1,4]oxazine-2-carbonyl]-4-(4-fluoro-benzyl)-piperidine-4-carbonitrile. LC/MS [M+H]⁺: 553.2. ¹H-NMR (400 MHz, DMSO-d₆) δ (ppm): 7.85 (d, 1H), 7.34 (d, 2H), 7.20 (m, 2H), 6.71 (m, 1H), 4.35 (m, 1H), 4.15 (m, 1H), 3.99 (s, 2H), 3.17-1.52 (14H). HPLC: 92.1%.

### Example 104

### 1-[6-Chloro-7-methoxy-4-(2,2,2-trifluoro-acetyl)-3,4-dihydro-2H-benzo[1,4]oxazine-2-carbonyl]-4-(4-fluoro-benzyl)-piperidine-4-carbonitrile

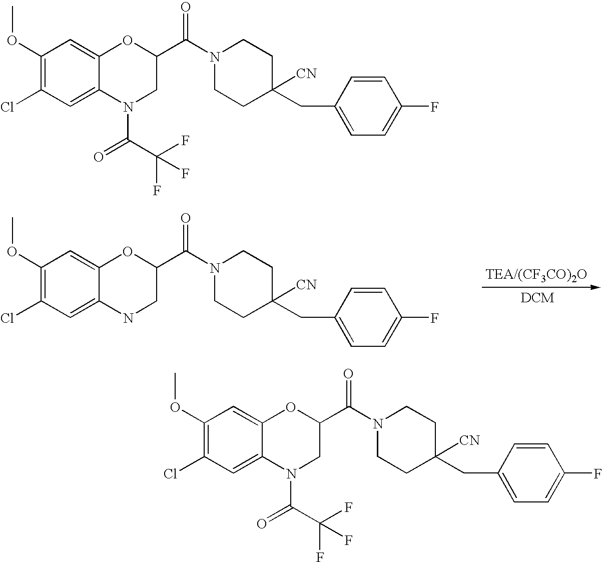

To a stirred solution of 1-(6-Chloro-7-methoxy-3,4-dihydro-2H-benzo[1,4]oxazine-2-carbonyl)-4-(4-fluoro-benzyl)-piperidine-4-carbonitrile (0.100g, 0.225 m.mol) in DCM was added TEA (0.094 ml, 0.675 m.mol) and trifluoro acetic anhydride (0.04 ml, 0.293 m.mol) drop wise in ice-cold condition. The reaction mixture was stirred for 4 h at room temperature. Concentrated thereaction mixture under reduced pressure and diluted with water. The organics was extracted with ethyl acetate, washed with water, brine, dried over Na₂SO4 and concentrated the solvent under reduced pressure. Thus obtained crude was purified with column chromatography (Si-gel, 17% ethyl acetate-hexane)to afford 57 mg (45.9%) of 1-[6-Chloro-7-methoxy-4-(2,2,2-trifluoro-acetyl)-3,4-dihydro-2H-benzo[1,4]oxazine-2-carbonyl]-4-(4-fluoro-benzyl)-piperidine-4-carbonitrile. ¹H NMR-(CR783-3506-34 in DMSO-d₆)-7.88(d, 1H), 7.37-7.17(m, 4H), 6.8(d, 1H), 5.62(m, 1H), 4.37-4.0(m, 4H), 3.86(d, 3H), 2.96-2.67(m, 3H), 1.9-1.5(m, 5H). LCMS (M+H)-540.2. HPLC-95.77%

### Example 105

### 1-(6-Chloro-7-methoxy-3-oxo-3,4-dihydro-2H-benzo[1,4]oxazine-2-carbonyl)-4-(4-fluoro-benzyl)-piperidine-4-carbonitrile

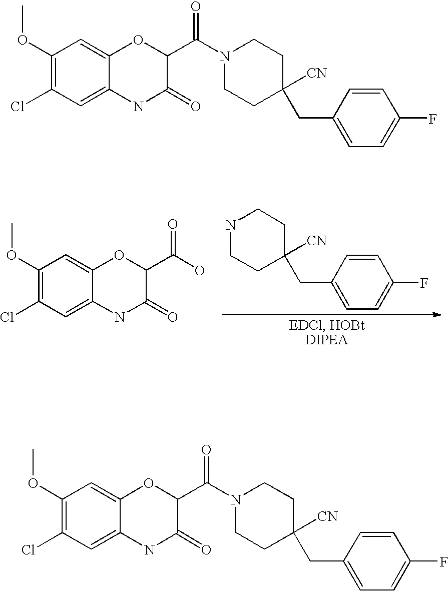

To a solution of 6-Chloro-7-methoxy-3-oxo-3,4-dihydro-2H-benzo[1,4]oxazine-2-carboxylic acid (150 mg, 0.58 mmol) in DMF was added 4-(4-Fluoro-benzyl)-piperidine-4-carbonitrile (127 mg, 0.58 mmol), EDCI (168 mg, 0.87 mmol), HOBt (95 mg, 07 mmol) and DIPEA (0.31 ml, 1.75 mmol) at 5-10° C. The reaction mixture was stirred at room temperature for 15 hours then diluted with water and extracted with ethyl acetate. The ethyl acetate layer was concentrated in vacuo and subjected to column chromatography (Si-gel, 20% EtOAC-hexane) to afford 120 mg (45.1%) of 1-(6-Chloro-7-methoxy-3-oxo-3,4-dihydro-2H-benzo[1,4]oxazine-2-carbonyl)-4-(4-fluoro-benzyl)-piperidine-4-carbonitrile. LCMS (M+H): 458.2. ¹H-NMR (400 MHz, DMSO-d₆) δ (ppm): 10.8 (m, 1H), 7.36 (m, 2H), 7.20 (m, 2H), 7.16 (m, 2H), 5.87 (m, 1H), 4.32 (m, 2H), 3.80 (m, 3H), 3.21-1.70 (m, 8H). HPLC-96.3%.

### Example 106

### 1-(6-Chloro-7-imidazol-1-yl-3,4-dihydro-2H-benzo[1,4]oxazine-2-carbonyl)-4-(4-fluoro-benzyl)-piperidine-4-carbonitrile

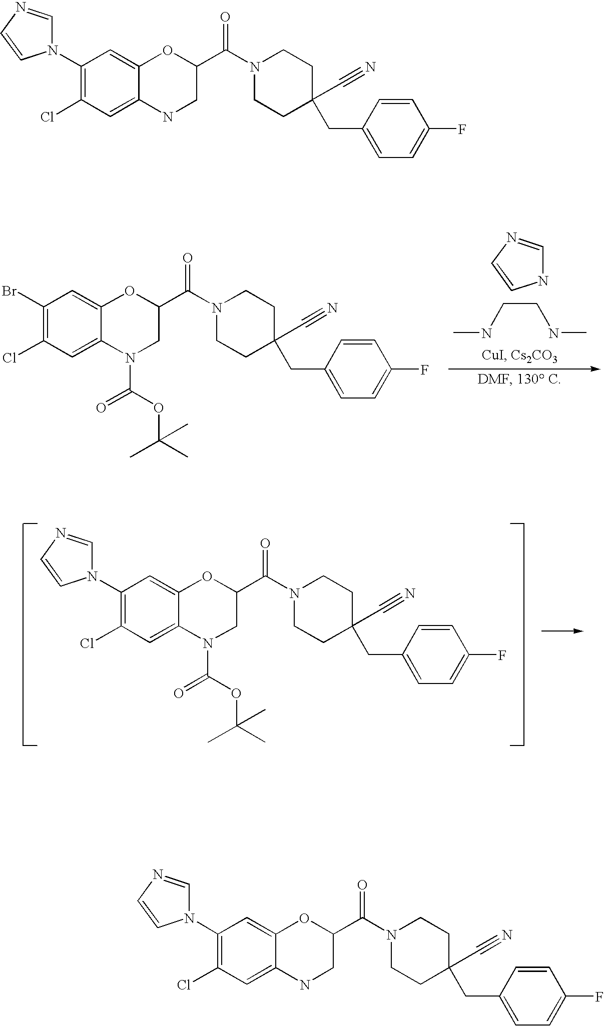

7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester was prepared by a process as described in example 57.
Step-1: 1-(6-Chloro-7-imidazol-1-yl-3,4-dihydro-2H-benzo[1,4]oxazine-2-carbonyl)-4-(4-fluoro-benzyl)-piperidine-4-carbonitrile

To a solution of 7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester (100 mg, 0.17 mmol) in DMF was added imidazole (14 mg, 0.2 mmol) and cesium carbonate (116 mg, 0.35 mmol). The solution was degassed with argon during 1 hour. To the previous degassed solution was added copper iodide (4 mg, 0.02 mmol) followed by N,N-dimethylethylenediamine (6 mg, 0.07 mmol) and was heated at 170° C. overnight. The reaction mixture was passed through a celite bed was diluted with water. The organics was extracted with ethyl acetate. The organic layer was concentrated and purified over column chromatography (Si-gel, 1% MeOH-DCM) to afford 12 mg (16.2%) of 1-(6-Chloro-7-imidazol-1-yl-3,4-dihydro-2H-benzo[1,4]oxazine-2-carbonyl)-4-(4-fluoro-benzyl)-piperidine-4-carbonitrile. LC/MS [M+H]⁺: 480.4. ¹H-NMR (400 MHz, DMSO-d₆) δ (ppm): 7.72 (bs, 1H0, 7.32-6.70 (m, 8H), 6.49 (s, 1H), 5.05 (m, 1H), 4.38 (m, 1H), 4.08 (m, 1H), 2.93 (m, 3H0, 1.90 (m, 4H), 1.77 (m, 3H). HPLC purity: 93.8%.

### Example 107

### 1-[6-Chloro-7-methoxy-4-(2H-tetrazol-5-yl)-3,4-dihydro-2H-benzo[1,4]oxazine-2-carbonyl]-4-(4-fluoro-benzyl)-piperidine-4-carbonitrile

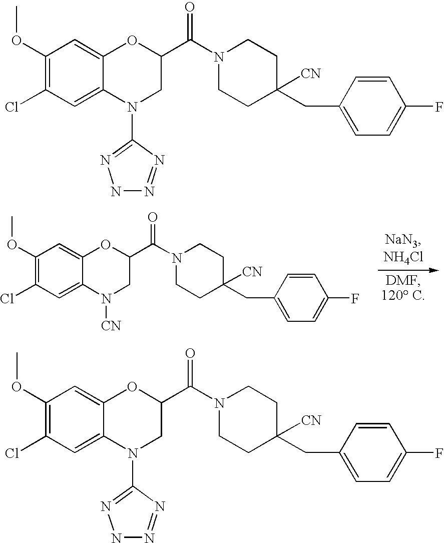

6-Chloro-2-[4-cyano-4-(4-fluoro-benzyl)-piperidine-1-carbonyl]-7-methoxy-2,3-dihydro-benzo[1,4]oxazine-4-carbonitrile was prepared by a process as described in example 81.

To a solution of 6-Chloro-2-[4-cyano-4-(4-fluoro-benzyl)-piperidine-1-carbonyl]-7-methoxy-2,3-dihydro-benzo[1,4]oxazine-4-carbonitrile in DMF was added NaN₃ (44 mg, 0.682 mmol) and NH₄Cl (36 mg, 0.682 mmol) and heated in a sealed tube at 120° C. for 16 hours. Then the reaction mixture was diluted with water, extracted ethyl acetate, washed with brine, dried over sodium sulfate and concentrated under reduced pressure followed by washing with hexane to afford 25 mg (28%) of 1-[6-Chloro-7-methoxy-4-(2H-tetrazol-5-yl)-3,4-dihydro-2H-benzo[1,4]oxazine-2-carbonyl]-4-(4-fluoro-benzyl)-piperidine-4-carbonitrile. LC/MS [M+H]⁺: 512.5. ¹H-NMR (400 MHz, DMSO-d₆) δ (ppm): 8.02 (m, 1H), 7.33 (bs, 2H), 7.19 (bs, 2H), 6.78 (m, 1H), 5.73 (s, 1H), 4.15 (m, 4H), 3.81 (s, 3H), 2.93 (m, 2H), 2.66 (m, 1H), 1.80 (m, 3H), 1.60 (m, 1H), 1.28(m, 1H). HPLC: 96%.

### Example 108

### 4-(4-Fluoro-benzyl)-1-(quinoxaline-2-carbonyl)-piperidine-4-carbonitrile

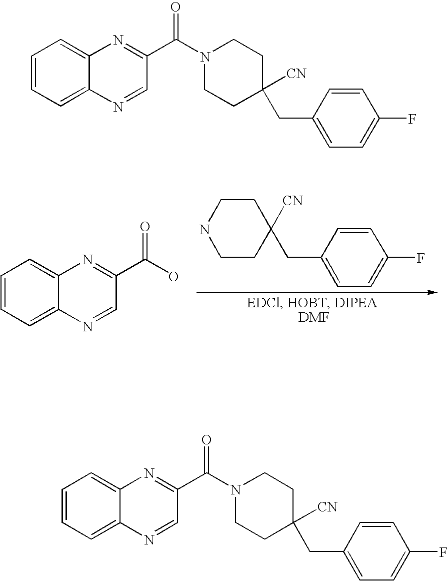

To a stirred solution of Quinoxaline-2-carboxylic acid (100 mg, 0.57 mmol) in DMF was added DIPEA (0.3 ml, 0.72 mmol), EDCI (220 mg, 1.15 mmol), and HOBT (116 mg, 0.86 mmol) at 0° C. The reaction mixture was stirred for half an hour atroom temperature and was added 4-(4-Fluoro-benzyl)-piperidine-4-carbonitrile (125 mg, 0.57 mmol). The reaction mixture was stirred overnight atroom temperature, diluted with water and extracted with ethyl acetate. The organic layer was washed with water, brine, dried over Na₂SO₄ and concentrated under reduced pressure. Thus obtained crude was purified with column chromatography (Si-gel, 0.5% MeOH-DCM) to afford 110 mg (51.4%) of 4-(4-Fluoro-benzyl)-1-(quinoxaline-2-carbonyl)-piperidine-4-carbonitrile. LC/MS [M+H]⁺: 375.4. ¹¹H-NMR (400 MHz, DMSO-d₆) δ (ppm): 9.1 (s, 1H), 8.17-7.94 (m, 4H), 7.36 (t, 2H), 7.17 (t, 2H), 4.64 (d, 1H), 4.0 (d, 1H), 3.27 (m, 1H), 2.99 (m, 3H), 1.93 (m, 1H), 1.79 (m, 3H). HPLC: 97.1%

### Example 109

### 4-(4-Fluoro-benzyl)-1-([1,2,4]triazolo[1,5-a]pyrimidine-2-carbonyl)-piperidine-4-carbonitrile

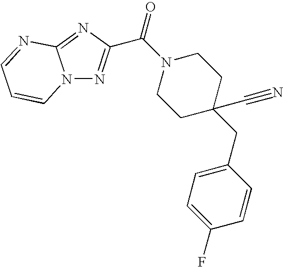

4-(4-Fluoro-benzyl)-1-([1,2,4]triazolo[1,5-a]pyrimidine-2-carbonyl)-piperidine-4-carbonitrile was prepared by a process similar to that described in example 108.

LC/MS [M+H]⁺: 365.2. ¹¹H-NMR (400 MHz, DMSO-d₆) δ (ppm): 9.46 (m, 1H), 8.96 (m, 1H), 7.47 (m, 1H), 7.35 (m, 2H), 7.18 (m, 2H), 4.59 (d, 1H), 4.05 (d, 1H), 3.17 (m, 1H), 2.95 (m, 3H), 1.94 (m, 1H), 1.73 (m, 3H). HPLC: 99.6%

### Example 110

### 4-(4-Fluoro-benzyl)-1-(pyrazolo[1,5-a]pyrimidine-3-carbonyl)-piperidine-4-carbonitrile

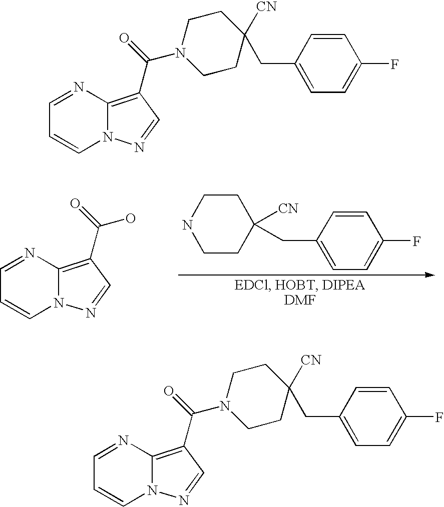

4-(4-Fluoro-benzyl)-1-(pyrazolo[1,5-a]pyrimidine-3-carbonyl)-piperidine-4-carbonitrile was prepared by a process similar to that described in example 108. LCMS (M+H)-364.4. ¹H NMR-(CR783-3506-56 in DMSO-d₆)-9.22(d, 2H), 8.71(d, 1H), 8.42(s, 1H), 7.37-7.16(m, 5H), 4.5(m, 2H), 4.0(m, 2H), 2.96(s, 2H), 1.81-1.74(m, 4H). HPLC-99.88%.

### Example 111

### 6-Chloro-2-[4-cyano-4-(4-fluoro-benzyl)-piperidine-1-carbonyl]-2,3-dihydro-benzo[1,4]oxazine-4-carbonitrile

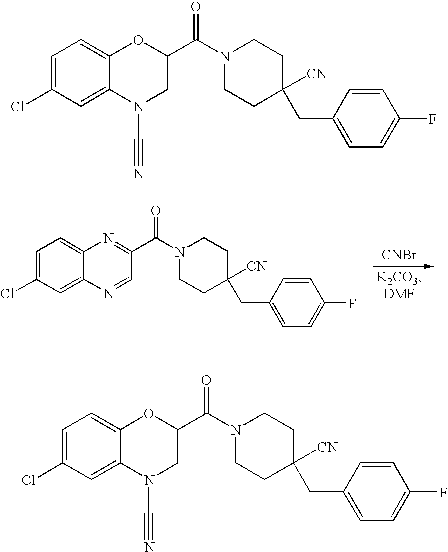

To a stirred solution of 1-(6-Chloro-3,4-dihydro-2H-benzo[1,4]oxazine-2-carbonyl)-4-(4-fluoro-benzyl)-piperidine-4-carbonitrile) (330 mg, 0.797 mmol) in DMF were added K₂CO₃ (330 mg, 2.39 mmol) and cyanogens bromide (126 mg, 1.19 mmol) in ice cool condition. The reaction mixture was stirred for 16 hours at room temperature and then it was diluted with water, extracted with ethyl acetate, washed with brine, dried over sodium sulfate and concentrated under resduced pressure to get the crude mass which was then purified by silica gel column chromatography (Si-gel, 20% ethyl acetate in hexane) to afford 160 mg (46%) of 6-Chloro-2-[4-cyano-4-(4-fluoro-benzyl)-piperidine-1-carbonyl]-2,3-dihydro-benzo[1,4]oxazine-4-carbonitrile. LC/MS [M+H]⁺: 439.2. ¹H-NMR (400 MHz, DMSO-d₆) δ (ppm): 7.34 (m, 2H), 7.19 (m, 2H), 7.01 (m, 3H), 5.37 (s, 1H), 4.35 (m, 1H), 4.12 (m, 1H), 3.98 (m, 2H), 3.22-1.47 (8H). HPLC: 94.2%

### Example 112

### 1-[6-Chloro-4-(2H-tetrazol-5-yl)-3,4-dihydro-2H-benzo[1,4]oxazine-2-carbonyl]-4-(4-fluoro-benzyl)-piperidine-4-carbonitrile

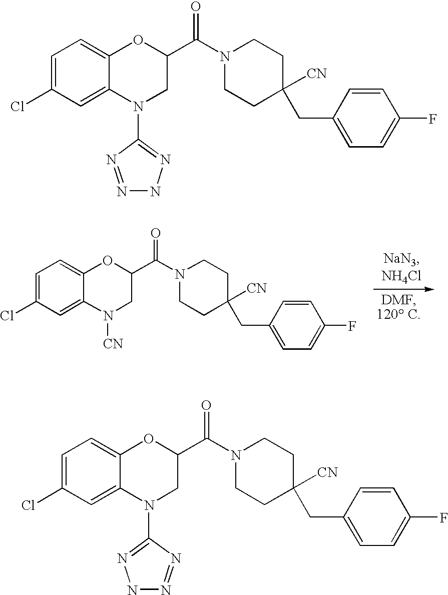

6-Chloro-2-[4-cyano-4-(4-fluoro-benzyl)-piperidine-1-carbonyl]-2,3-dihydro-benzo[1,4]oxazine-4-carbonitrile was prepared by a process as described in example 111.

To a solution of 6-Chloro-2-[4-cyano-4-(4-fluoro-benzyl)-piperidine-1-carbonyl]-2,3-dihydro-benzo[1,4]oxazine-4-carbonitrile (50 mg, 0.114 mmol) in DMF were added NH₄Cl (24 mg, 0.455 mmol) and NaN₃ (30 mg, 0.455 mmol). The reaction mixture was taken in a sealed tube and it was heated at 120° C. for 16 hours. Then it was diluted with water and extracted with water. The organic layer was separated and washed wth brine, dried over sodium sulfate and concentrated under reduced pressure to give the crude product which was washed with hexane to afford 15 mg (27%) of 1-[6-Chloro-4-(2H-tetrazol-5-yl)-3,4-dihydro-2H-benzo[1,4]oxazine-2-carbonyl]-4-(4-fluoro-benzyl)-piperidine-4-carbonitrile. LC/MS [M+H]⁺: 482.3. ¹H-NMR (400 MHz, DMSO-d₆) δ (ppm): 8.36 (m, 1H), 7.30 (m, 2H), 7.16 (m, 2H), 6.86 (d, 1H), 6.77 (bs, 1H), 5.20 (m, 1H), 4.32 (m, 1H), 4.13 (m, 2H), 3.86 (m, 1H), 3.10-1.44 (8H). HPLC: 92.3%.

### Example 113

### 1-[6-Chloro-4-(1H-[1,2,4]triazol-3-yl)-3,4-dihydro-2H-benzo[1,4]oxazine-2-carbonyl]-4-(4-fluoro-benzyl)-piperidine-4-carbonitrile

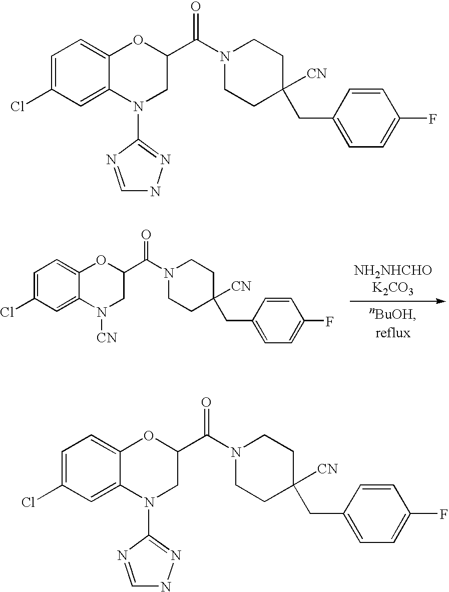

6-Chloro-2-[4-cyano-4-(4-fluoro-benzyl)-piperidine-1-carbonyl]-2,3-dihydro-benzo[1,4]oxazine-4-carbonitrile was prepared by a process as described in example 111.

To a stirred solution of 6-Chloro-2-[4-cyano-4-(4-fluoro-benzyl)-piperidine-1-carbonyl]-2,3-dihydro-benzo[1,4]oxazine-4-carbonitrile (90 mg, 0.21 mmol) in n-butanol (7 ml) were added NH₂NHCHO (16 mg, 0.27 mmol) and K₂CO₃ (56 mg, 0.41 mmol) in ice cool condition. Then the reaction mixture was refluxed for 16 hours. The reaction mixtures was concentrated and then diluted with ethyl acetate and water. The organic layer was separated, washed with brine, dried over sodium sulfate and concentrated under reduced pressure to afford the crude mass which was then purified by column chromatography (Si-gel, 5% methanol—DCM) to afford 20 mg (20%) of 1-[6-Chloro-4-(1H-[1,2,4]triazol-3-yl)-3,4-dihydro-2H-benzo[1,4]oxazine-2-carbonyl]-4-(4-fluoro-benzyl)-piperidine-4-carbonitrile. LC/MS [M+H]⁺: 479.4. ¹H-NMR (400 MHz, DMSO-d₆) δ (ppm): 13.64 (s, 1H), 8.42 (m, 1H), 7.34 (bs, 2H), 7.19 (m, 2H), 6.89 (m, 2H), 5.25 (m, 1H), 4.36-3.96 (m, 4H), 3.20-1.38 (m, 8H). HPLC: 96.1%.

### Example 114

### 1-(6-Chloro-7-pyrrol-1-yl-3,4-dihydro-2H-benzo[1,4]oxazine-2-carbonyl)-4-(4-fluoro-benzyl) piperidine-4-carbonitrile

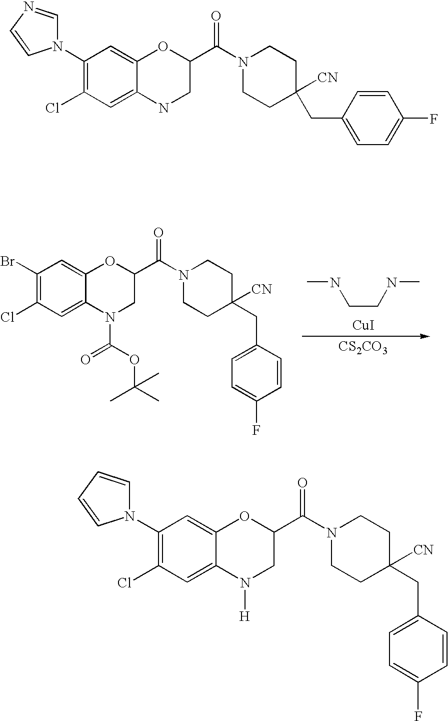

7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester was prepared by a process as described in example 57.

To a solution of 7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester (200 mg, 0.34 mmol) in DMF was added Pyrrole (26 mg, 0.38 mmol) in a sealed tube. The reaction mixture was degassed with Argon for about 15 min followed by addition of cesium carbonate (231 mg, 0.71 mmol), copper iodide (13 mg, 0.07 mmol) and N,N dimethyl ethylenediammine (12 mg, 0.135 mmol). The reaction mixture was heated at 170° C. overnight. The reaction mixture was filtered through celite bed and purified over column chromatography (Si-gel, 1.5% MeOH-DCM) to afford 26 mg (16%) of 1-(6-Chloro-7-pyrrol-1-yl-3,4-dihydro-2H-benzo[1,4]oxazine-2-carbonyl)-4-(4-fluoro-benzyl) piperidine-4-carbonitrile. LC/MS [M+H]⁺: 479.4. ¹H-NMR (400 MHz, DMSO-d₆) δ (ppm): 7.33 (2H, bs), 7.19(2H, bs), 6.83 (3H, m), 6.37 (3H, bs), 5.02 (1H, bs), 4.38 (1H, bs), 4.09 (1H, bs), 3.44 (1H,m), 3.34 (2H, s), 2.93 (2H, m), 2.68 (1H, m), 1.85 (3H, m), 1.77 (1H, m). HPLC purity: 91.1%.

### Example 115

### 1-[6-Chloro-7-methoxy-4-(1H-[1,2,4]triazol-3-yl)-3,4-dihydro-2H-benzo[1,4]oxazine-2-carbonyl]-4-(4-fluoro-benzyl)-piperidine-4-carbonitrile

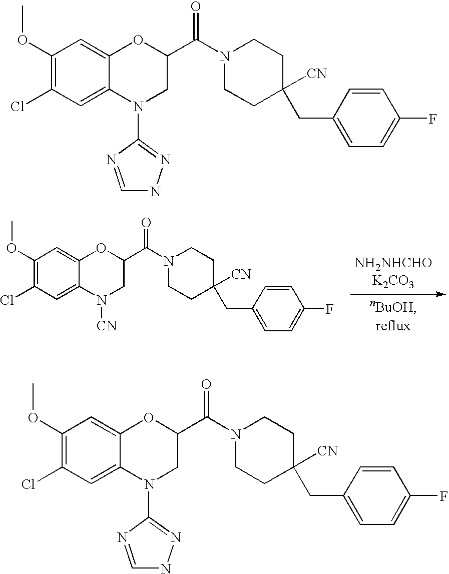

6-Chloro-2-[4-cyano-4-(4-fluoro-benzyl)-piperidine-1-carbonyl]-7-methoxy-2,3-dihydro-benzo[1,4]oxazine-4-carbonitrile was prepared as described in example 81.

To a stirred solution of 6-Chloro-2-[4-cyano-4-(4-fluoro-benzyl)-piperidine-1-carbonyl]-7-methoxy-2,3-dihydro-benzo[1,4]oxazine-4-carbonitrile)(100 mg, 0.21 mmol) in n-butanol (7 ml) were added NH₂NHCHO (16 mg, 0.27 mmol) and K₂CO₃(59 mg, 0.426 mmol) in ice cool condition. Then the reaction mixture was subjected to reflux for 16 hours. Then the reaction mixture was concentrated and then diluted with ethyl acetate and water. Organic layer was separated, washed with brine, dried over sodium sulfate and concentrated under reduced pressure to give the crude mass which was then purified by silica gel column chromatography (5% methanol-DCM) to afford 10 mg (10%) of 1-[6-Chloro-7-methoxy-4-(1H-[1,2,4]triazol-3-yl)-3,4-dihydro-2H-benzo[1,4]oxazine-2-carbonyl]-4-(4-fluoro-benzyl)-piperidine-4-carbonitrile. LC/MS [M+H]⁺: 511.4. ¹H-NMR (400 MHz, DMSO-d₆) δ (ppm): 13.51 (s, 1H), 8.35 (m, 1H), 7.34 (bs, 2H), 7.21(m, 2H), 6.71(m, 1H), 6.44 (m, 1H), 5.26 (m, 1H), 4.36 (m, 1H), 4.15 (m, 2H), 3.79 (s, 3H), 3.19-1.39 (m, 8H). HPLC: 75.2%.

### Example 116

### 1-[6-Chloro-8-(1H-pyrazol-4-yl)-3,4-dihydro-2H-benzo[1,4]oxazine-2-carbonyl]-4-(4-fluoro-benzyl)-piperidine-4-carbonitrile

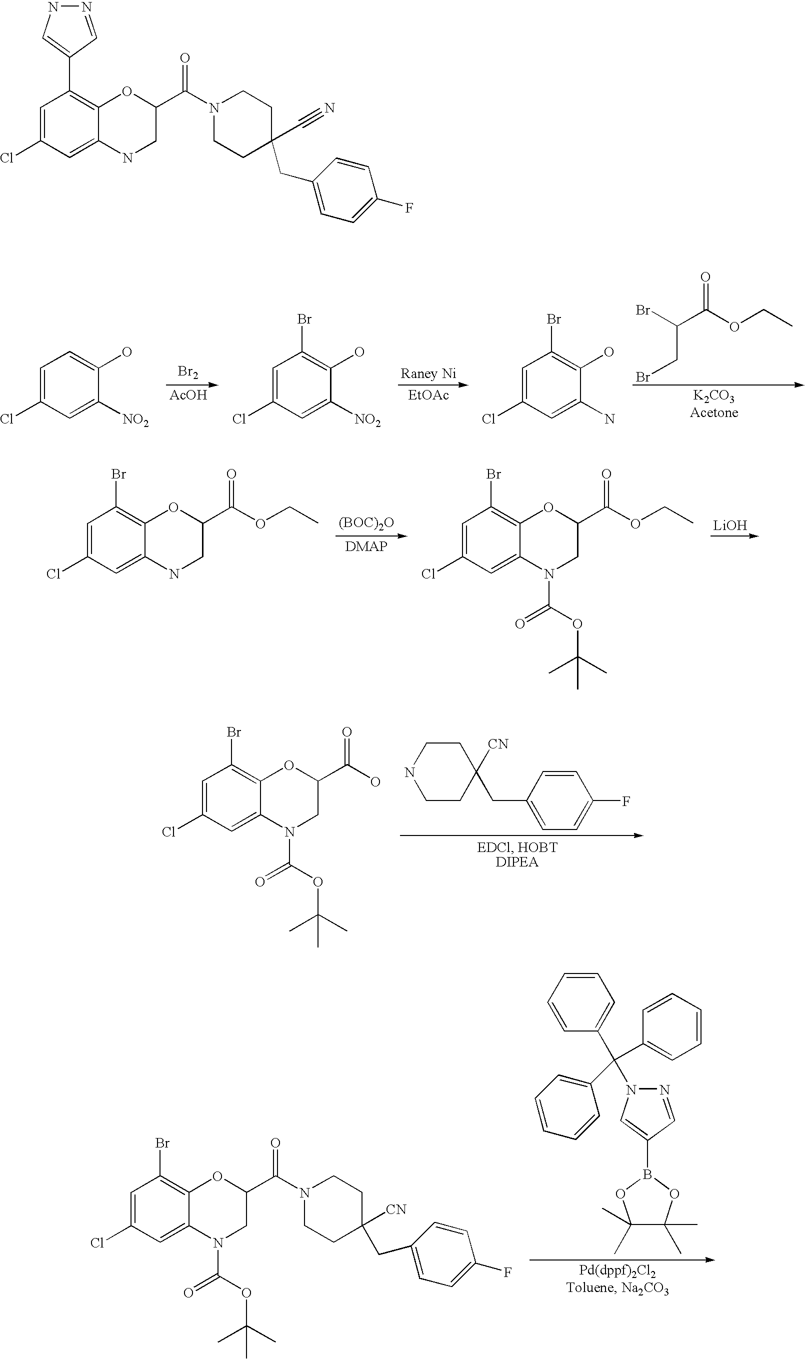

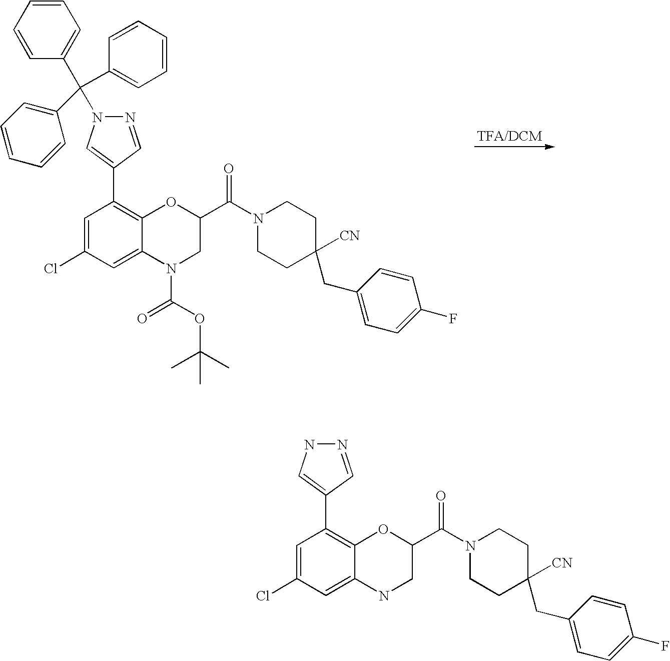

Step-1: 2-Bromo-4-chloro-6-nitro-phenol

To a solution of 4-chloro-2-nitrophenol (1 g, 5.76 mmol) in acetic acid was added bromine (0.32 ml, 6.3 mmol) at 5-10° C. and stirred at 10° C. for half an hour. The temperature of the reaction mixture was raised to room temperature and stireed at RT for 3 hours. The reaction mixture was diluted with ice-water and extracted with ethyl acetate. The organic layer was washed with ethyl acetate and dried over sodium sulfate. Concentration of organic layer was afforded 1.3 g (90.9%) of 2-Bromo-4-chloro-6-nitro-phenol. GCMS [m/z]: 253.0. ¹H-NMR (400 MHz, DMSO-d₆) δ (ppm): 11.1 (s, 1H), 8.10 (d, 1H), 8.04 (d, 1H).

Step-2: 2-Amino-6-bromo-4-chloro-phenol

To a solution of 2-bromo-4-chloro-6-nitro-phenol (2.7 g, 10.6 mmol) in ethyl acetate was added raney Ni (1 g) and was hydrogenetaed at ballon pressure for 4 hours. The reaction mixture was filtered through celite bed and mother liquor was concentrated to afford 2 g (85.1%) of 2-Amino-6-bromo-4-chloro-phenol. LCMS [M+1]: 224.0. ¹H-NMR (400 MHz, DMSO-d₆) δ (ppm): 6.67 (s, 1H), 6.61 (s, 1H), 6.57 (s, 1H), 5.2 (bs, 2H).

Step-3: 8-Bromo-6-chloro-3,4-dihydro-2H-benzo[1,4]oxazine-2-carboxylic acid ethyl ester

2-Amino-6-bromo-4-chloro-phenol (360 mg, 1.62 mmol) was dissolved in acetone. Anhydrous K₂CO₃ (603.7 mg, 4.37 mmol) and diethyl-2-bromomalonate (0.25 ml, 1.8 mmol) were added to the solution and the resulting mixture was refluxed overnight. The reaction mixture was concentrated, diluted with ice-water and extracted with ethyl acetate. The organic layer was washed successively with water and brine and then dried over Na₂SO₄. The organic layer was concentrated under reduced pressure and purification of the residue by flash chromatography (silica-gel, 5% EtOAc-hexane) afforded 270 mg (52.1%) of 8-Bromo-6-chloro-3,4-dihydro-2H-benzo[1,4]oxazine-2-carboxylic acid ethyl ester. LCMS [M+1]: 322.2. ¹H-NMR (400 MHz, DMSO-d₆) δ (ppm): 6.81 (d, 1H), 6.59 (d, 1H), 6.45 (s, 1H), 5.14 (t, 1H), 4.01 (q, 2H), 3.49 (m, 1H), 3.47 (d, 1H), 1.16 (t, 3H).

Step-4: 8-Bromo-6-chloro-2,3-dihydro-benzo[1,4]oxazine-2,4-dicarboxylic acid 4-tert-butyl ester 2-ethyl ester

DMAP (57 mg, 4.6 mmol) and (Boc)₂O (1 ml, 5.0 mmol) were added to a solution of 8-Bromo-6-chloro-3,4-dihydro-2H-benzo[1,4]oxazine-2-carboxylic acid ethyl ester (1.5 g, 4.6 mmol) in THF and the resulting mixture was heated to reflux overnight. The mixture was then concentrated, diluted with water and extracted with ethyl acetate. The organic layer was dried over sodium sulfate and concentrated under reduced pressure. The crude product was purified through column chromatography over Si-gel (6%-ethyl acetate-hexane) to afford 1.2 g (63.1%) of 8-Bromo-6-chloro-2,3-dihydro-benzo[1,4]oxazine-2,4-dicarboxylic acid 4-tert-butyl ester 2-ethyl ester. ¹H-NMR (400 MHz, DMSO-d₆) δ (ppm): 7.76 (s, 1H), 7.47 (s, 1H), 5.38 (s, 1H), 4.51 (d, 1H), 4.11 (m, 2H), 3.57 (d, 1H), 1.45 (s, 9H), 1.20 (t, 3H).

Step-5: 8-Bromo-6-chloro-2,3-dihydro-benzo[1,4]oxazine-2,4-dicarboxylic acid 4-tert-butyl ester

To a solution of 8-Bromo-6-chloro-2,3-dihydro-benzo[1,4]oxazine-2,4-dicarboxylic acid 4-tert-butyl ester 2-ethyl ester (1 gm, 2.4 mmol) in (4:1) THF: H2O was added (110 mg, 2.6 mmol) LiOH. The reaction mixture was stirred at room tempearture for 3 hours. The reaction mixture was concentrated, diluted with water, neutralized with acetic acid and extracted with ethyl acetate. The organic layer was washed with water and concentrated under reduced pressure to afford 920 mg (98.9%) of 8-Bromo-6-chloro-2,3-dihydro-benzo[1,4]oxazine-2,4-dicarboxylic acid 4-tert-butyl ester. ¹H-NMR (400 MHz, DMSO-d₆) δ (ppm): 13.5 (brs, 1H), 7.72 (s, 1H), 7.44 (s, 1H), 5.23 (s, 1H), 4.48 (m, 1H), 3.54 (m, 1H), 1.39 (s, 9H).

Step-6: 8-Bromo-6-chloro-2-[4-cyano-4-(4-fluoro-benzyl)-piperidine-1-carbonyl]-2,3-dihydro-benzo[1,4]oxazine-4-carboxylic acid tent-butyl ester

To a solution of 8-Bromo-6-chloro-2,3-dihydro-benzo[1,4]oxazine-2,4-dicarboxylic acid 4-tert-butyl ester (1.1 gm, 2.8 mmol) in DMF was added DIPEA (1.6 ml, 8.4 mmol) at cold condition followed by addition of EDCI (805 mg, 4.2 mmol) and HOBT (380 mg, 2.8 mmol). The reacting solution was stirred for 30 minutes and was added 4-(4-Fluoro-benzyl)-piperidine-4-carbonitrile (615 mg, 2.8 mmol). The reaction mixture was stirred at room temperature overnight. The reaction mixture was diluted with water and extracted with ethyl acetate. The organic layer was washed with water, concentrated and purified over column chromatography (Si-gel, 15% ethyl acetate-hexane) to afford 1.3 g (78.3%) of 8-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester. ¹H-NMR (400 MHz, DMSO-d₆) δ (ppm): 7.69-7.17 (m, 6H), 5.57 (m, 1H), 4.36-1.43 (m, 21H).

Step-7: 6-Chloro-2-[4-cyano-4-(4-fluoro-benzyl)-piperidine-1-carbonyl]-8-(1-trityl-1H-pyrazol-4-yl)-2,3-dihydro-benzo[1,4]oxazine-4-carboxylic acid tert-butyl ester

To a solution of 8-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester (100 mg, 0.17 mmol) in 3:1 toluene-water was added 4-(4,4,5,5-Tetramethyl-[1,3,2]dioxaborolan-2-yl)-1-trityl-1H-pyrazole (74 mg, 0.17 mmol) and Na₂CO₃ (36 mg, 0.34 mmol). The solution was degassed with nitrogen for 1 hour and was added catalytic amount of Pd(dppf)₂Cl₂ at argon atmosphere. The reaction mixture was heated at 90° C. for overnight, diluted with water and extracted with ethyl acetate. The organic layer was washed with water, brine and dried over Na₂SO₄. The organics was concentrated and purified purified over column chromatography (Si-gel, 15% ethyl acetate-hexane) to afford 110 mg (78.7%) of 6-Chloro-2-[4-cyano-4-(4-fluoro-benzyl)-piperidine-1-carbonyl]-8-(1-trityl-1H-pyrazol-4-yl)-2,3-dihydro-benzo[1,4]oxazine-4-carboxylic acid tert-butyl ester. ¹H-NMR (400 MHz, DMSO-d₆) δ (ppm): 8.15-7.05 (m, 23H), 5.45-1.79 (m, 13H), 1.43 (s, 9H).

### Step-8

To a solution of 6-Chloro-2-[4-cyano-4-(4-fluoro-benzyl)-piperidine-1-carbonyl]-8-(1-trityl-1H-pyrazol-4-yl)-2,3-dihydro-benzo[1,4]oxazine-4-carboxylic acid tert-butyl ester (100 mg, 0.12 mmol) in DCM was added TFA (2 ml) and stirred at RT for 4 hours. The DCM solution was concentrated, diluted with water and basified with dilute NaOH solution. The organics was extracted with ethyl acetate, washed with water. The organic layer was concentrated and purified over column chromatography (Si-gel, 1% MeOH-DCM) to afford 55 mg (95.5%) of 1-[6-Chloro-8-(1H-pyrazol-4-yl)-3,4-dihydro-2H-benzo[1,4]oxazine-2-carbonyl]-4-(4-fluoro-benzyl)-piperidine-4-carbonitrile. LC/MS [M+H]⁺: 480.2. ¹H-NMR (400 MHz, DMSO-d₆) δ (ppm): 12.93 (m, 1H), 8.13-6.19 (m, 9H), 4.94 (m, 1H), 4.42 (m, 1H), 3.96 (m, 1H), 3.47-1.54 (m, 10H). HPLC: 95.2%.

### Example 117

### 1-(6-Chloro-8-pyridin-4-yl-3,4-dihydro-2H-benzo[1,4]oxazine-2-carbonyl)-4-(4-fluoro-benzyl)-piperidine-4-carbonitrile

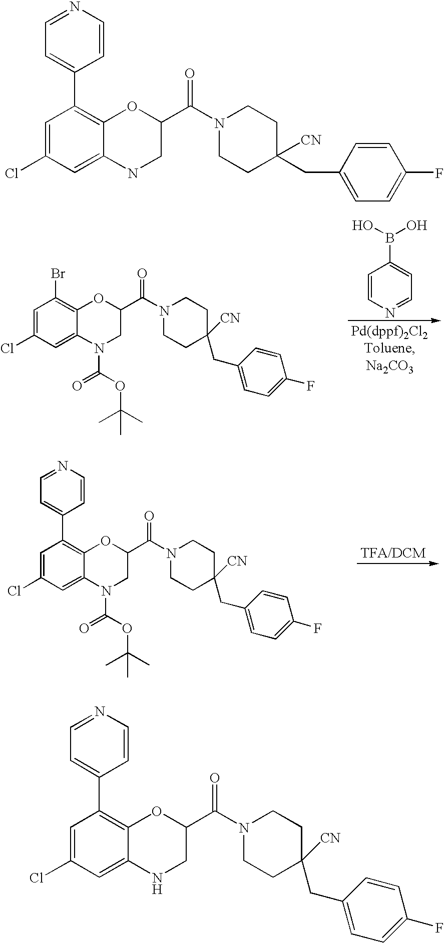

8-Bromo-6-chloro-2-[4-cyano-4-(4-fluoro-benzyl)-piperidine-1-carbonyl]-2,3-dihydro-benzo[1, 4]oxazine-4-carboxylic acid tert-butyl ester was prepared by a process as described in example 116.
Step-1: 6-Chloro-2-[4-cyano-4-(4-fluoro-benzyl)-piperidine-1-carbonyl]-8-pyridin-4-yl-2,3-dihydro-benzo[1,4]oxazine-4-carboxylic acid tert-butyl ester

To a solution of 8-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester (100 mg, 0.17 mmol) in 3:1 toluene-water was added pyridine-4-boronic acid (21 mg, 0.17 mmol), Na₂CO₃ (36 mg, 0.34 mmol). The solution was degassed with nitrogen for 1 hour and was added catalytic amount of Pd(dppf)₂Cl₂ at argon atmosphere. The reaction mixture was heated at 90° C. for overnight, diluted with water and extracted with ethyl acetate. The organic layer was washed with water, brine and dried over Na₂SO₄. The organics was concentrated and purified purified over column chromatography (Si-gel, 1.5% MeOH-DCM) to afford 70 mg (69.7%) of 6-Chloro-2-[4-cyano-4-(4-fluoro-benzyl)-piperidine-1-carbonyl]-8-pyridin-4-yl-2,3-dihydro-benzo[1,4]oxazine-4-carboxylic acid tert-butyl ester. LCMS [M+H]⁺: 491.2.

### Step-2

To a solution of 6-Chloro-2-[4-cyano-4-(4-fluoro-benzyl)-piperidine-1-carbonyl]-8-pyridin-4-yl-2,3-dihydro-benzo[1,4]oxazine-4-carboxylic acid tert-butyl ester (70 mg, 0.11 mmol) in DCM was added TFA (1 ml) and stirred at RT for 3 hours. The DCM solution was concentrated, diluted with water and basified with dilute NaOH solution. The organics was extracted with ethyl acetate, washed with water. The organic layer was concentrated and washed with hexane to afford 30 mg (55.5%) of 1-(6-Chloro-8-pyridin-4-yl-3,4-dihydro-2H-benzo[1,4]oxazine-2-carbonyl)-4-(4-fluoro-benzyl)-piperidine-4-carbonitrile. LC/MS [M+H]⁺: 491.2. ¹H-NMR (400 MHz, DMSO-d₆) δ (ppm): 8.60 (m, 2H), 7.52 (d, 2H), 7.32-7.19 (m, 4H), 6.68-6.41(m, 3H), 5.01 (m, 1H), 4.37 (m, 1H), 3.84 (m, 1H), 3.41 (m, 2H), 3.02-1.18 (m, 814). HPLC: 98.8%

### Example 118

### 1-(6-Chloro-8-thiophen-3-yl-3,4-dihydro-2H-benzo[1,4]oxazine-2-carbonyl)-4-(4-fluoro-benzyl)-piperidine-4-carbonitrile

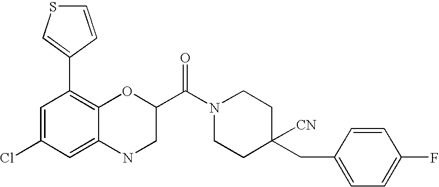

8-Bromo-6-chloro-2-[4-cyano-4-(4-fluoro-benzyl)-piperidine-1-carbonyl]-2,3-dihydro-benzo[1, 4]oxazine-4-carboxylic acid tert-butyl ester was prepared by a process as described in example 116.

1-(6-Chloro-8-thiophen-3-yl-3,4-dihydro-2H-benzo[1,4]oxazine-2-carbonyl)-4-(4-fluoro-benzyl)-piperidine-4-carbonitrile was prepared by a similar process i.e Suzuki coupling with thiophene-3-boronic acid followed by deprotection as described in example 117.

LC/MS [M+H]⁺: 496.4. ¹H-NMR (400 MHz, DMSO-d₆) δ (ppm): 7.78 (m, 1H), 7.61 (m, 1H), 7.45 (d, 1H), 7.30 (m, 2H), 7.19 (m, 2H), 6.74 (m, 1H), 6.57 (m, 1H), 6.30 (m, 1H), 4.97 (m, 1H), 4.41 (m, 1H), 3.93 (m, 1H), 3.34 (m, 2H), 2.95 (m, 3H), 2.66(m, 2H), 1.900.93 (m, 3H).

HPLC:89.5%.

### Example 119

### 1-(6-Chloro-7-thiophen-3-yl-3,4-dihydro-2H-benzo[1,4]oxazine-2-carbonyl)-4-(4-fluoro-benzyl)-piperidine-4-carbonitrile

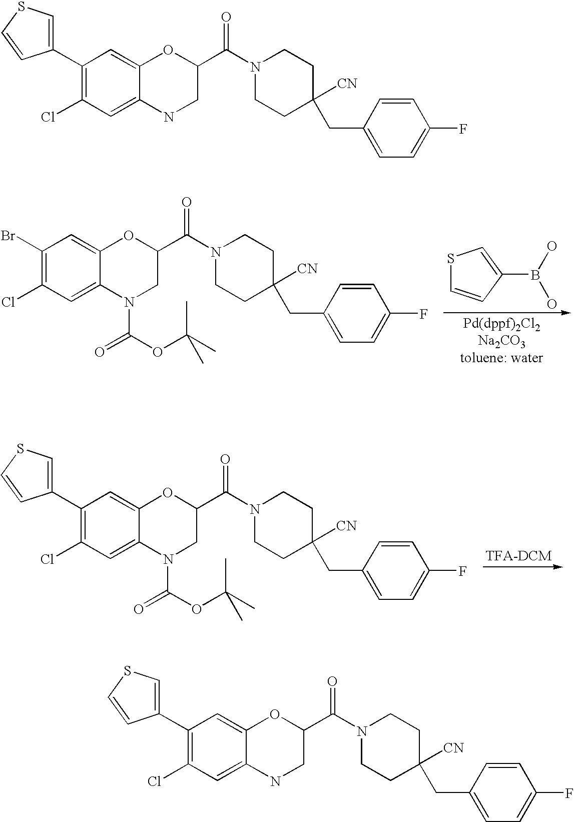

1-(7-Bromo-6-chloro-3,4-dihydro-2H-benzo[1,4]oxazine-2-carbonyl)-4-(4-fluoro-benzyl)-piperidine-4-carbonitrile was prepared by a process described in example 57.
Step-1: 6-Chloro-2-[4-cyano-4-(4-fluoro-benzyl)-piperidine-1-carbonyl]-7-thiophen-3-yl-2,3-dihydro-benzo[1,4]oxazine-4-carboxylic acid tent-butyl ester

To a stirred solution of 7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester (0.17 m.mol, 0.100 g) Na₂CO₃ (0.36 m.mol, 0.036 g) and Thiophene boronic acid (0.17 m.mol, 0.022 g) in toluene: water (3:1, degassed with Ar) were further degassed with Ar for 1 h, then [Pd(dppf)₂Cl₂] (25 mg) was added to it and heated the reaction mixture at 100° C. for overnight. Water was added to it. Extracted the compound with ethyl acetate, washed with water, brine, dried over Na₂SO4 and concentrated the solvent under reduced pressure. Thus obtained crude was purified with column chromatography using 100-200 mesh silica gel, 15% EA/Hex as Eluent to get (0.085g) of 6-Chloro-2-[4-cyano-4-(4-fluoro-benzyl)-piperidine-1-carbonyl]-7-thiophen-3-yl-2,3-dihydro-benzo[1,4]oxazine-4-carboxylic acid tert-butyl ester (6). LCMS (M+H)-496.2.

¹H NMR-(CR783-3506-72 in CDCl₃)-7.99-6.93(m, 6H), 4.93-3.35(m, 7H), 2.87(s, 2H), 2.03-1.66(m, 4H), 1.55(s, 9H).

Step-2: 1-(6-Chloro-7-thiophen-3-yl-3,4-dihydro-2H-benzo[1,4]oxazine-2-carbonyl)-4-(4-fluoro-benzyl)-piperidine-4-carbonitrile

To a stirred solution of 6-Chloro-2-[4-cyano-4-(4-fluoro-benzyl)-piperidine-1-carbonyl]-7-thiophen-3-yl-2,3-dihydro-benzo[1,4]oxazine-4-carboxylic acid tert-butyl ester. (0.143 m.mol, 0.085 g) in DCM (10 ml), TFA (1.43 m.mol, 0.11 ml) was added in ice-cold condition. Left the reaction mixture for 2 h at rt.concentrared the reaction mixture and diluted with water, basified the compound with dil NaOH solution Extracted the compound with Ethyl acetate, washed with water, brine, dried over Na₂SO4 and concentrated the solvent under reduced pressure, washed the compound with Hexane to get (0.030 g) of 1-(6-Chloro-7-thiophen-3-yl-3,4-dihydro-2H-benzo[1,4]oxazine-2-carbonyl)-4-(4-fluoro-benzyl)-piperidine-4-carbonitrile. LC/MS [M+H]⁺: 496.4. ¹H-NMR (400 MHz, DMSO-d₆) δ (ppm): 7.56 (m, 2M), 7.33 (m, 2H), 7.20 (m, 1H), 7.16 (m, 2H), 6.87 (m, 1H), 6.69 (s, 1H), 6.29 (s, 2H), 4.97 (m, 1H), 4.38 (m, 1H), 4.10 (m, 1H), 3.32 (m, 2H), 3.07 (m, 1H), 2.94 (s, 2H), 1.90 (m, 4H), 1.51 (m, 1H). HPLC: 94.1%.

### Example 120

### 1-(6-Chloro-7-pyridin-3-yl-3,4-dihydro-2H-benzo[1,4]oxazine-2-carbonyl)-4-(4-fluoro-benzyl)-piperidine-4-carbonitrile

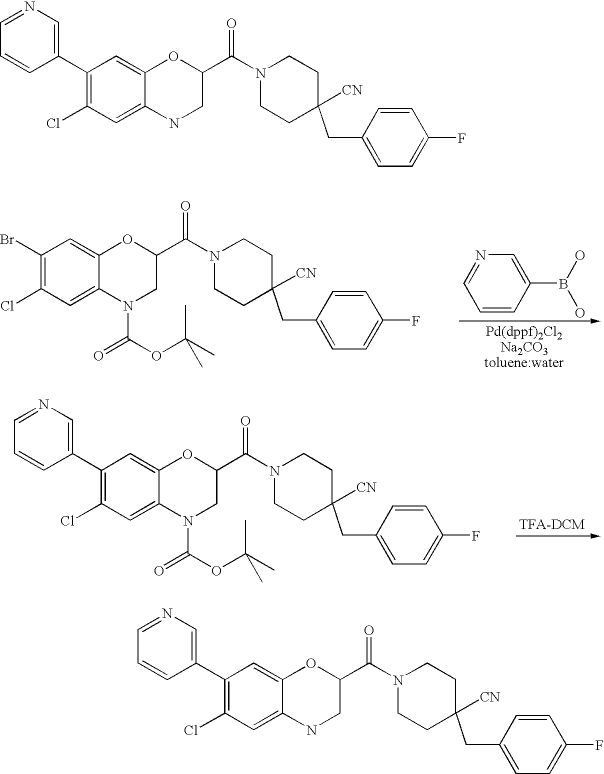

Step-1: 6-Chloro-2-[4-cyano-4-(4-fluoro-benzyl)-piperidine-1-carbonyl]-7-pyridin-3-yl-2,3-dihydro-benzo[1,4]oxazine-4-carboxylic acid tent-butyl ester

To a stirred solution of 7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester (0.17 m.mol, 0.100 g) Na₂CO₃ (0.36 m.mol, 0.036 g) and Pyridine boronic acid (0.17 m.mol, 0.021 g) in toluene: water (3:1, degassed with Ar) were further degassed with Ar for 1 h, then Pd(dppf)₂Cl₂ (25 mg) was added to it and heated the reaction mixture at 100° C. for overnight. Water was added to it. Extracted the compound with ethyl acetate, washed with water, brine, dried over Na₂SO4 and concentrated the solvent under reduced pressure. Thus obtained crude was purified with column chromatography using 100-200 mesh silica gel, 25% EA/Hex as Eluent to get (0.067 g) of of 6-Chloro-2-[4-cyano-4-(4-fluoro-benzyl)-piperidine-1-carbonyl]-7-pyridin-3-yl-2,3-dihydro-benzo[1,4]oxazine-4-carboxylic acid tert-butyl ester (6). ¹H NMR-(CR783-3506-74 in CDCl₃)-8.66-6.85(m, 10H), 4.97-3.39(m, 7H), 2.87(s, 2H), 2.09-1.64(m, 4H), 1.55(s, 9H). LCMS (M+H)-591.2.

Step-2: 1-(6-Chloro-7-pyridin-3-yl-3,4-dihydro-2H-benzo[1,4]oxazine-2-carbonyl)-4-(4-fluoro-benzyl)-piperidine-4-carbonitrile

To a stirred solution of 6-Chloro-2-[4-cyano-4-(4-fluoro-benzyl)-piperidine-1-carbonyl]-7-pyridin-3-yl-2,3-dihydro-benzo[1,4]oxazine-4-carboxylic acid tert-butyl ester (0.11 m.mol, 0.067 g) in DCM (10 ml), TFA (1.1 m.mol, 0.085 ml) was added in ice-cold condition. Left the reaction mixture for 2 h at rt.concentrared the reaction mixture and diluted with water, basified the compound with dil NaOH solution Extracted the compound with Ethyl acetate, washed with water, brine, dried over Na₂SO4 and concentrated the solvent under reduced pressure, washed the compound with Hexane to get (0.014 g) of 1-(6-Chloro-7-pyridin-3-yl-3,4-dihydro-2H-benzo[1,4]oxazine-2-carbonyl)-4-(4-fluoro-benzyl)-piperidine-4-carbonitrile. LC/MS [M+H]⁺: 491.4. ¹H-NMR (400 MHz, DMSO-d₆) δ (ppm): 8.57-6.76(m, 10H), 6.41(s, 1H), 4.39-2.67(m, 8H), 1.86-1.54(m, 5H). HPLC purity: 94.7%.

### Example 121

### 1-(6-Chloro-7-pyridin-4-yl-3,4-dihydro-2H-benzo[1,4]oxazine-2-carbonyl)-4-(4-fluoro-benzyl)-piperidine-4-carbonitrile

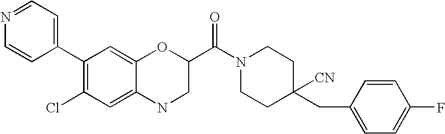

1-(6-Chloro-7-pyridin-4-yl-3,4-dihydro-2H-benzo[1,4]oxazine-2-carbonyl)-4-(4-fluoro-benzyl)-piperidine-4-carbonitrile was prepared by the similar procedure as described in example 120. LC/MS [M+H]⁺: 491.4. ¹H-NMR (400 MHz, DMSO-d₆) δ (ppm): 8.58 (bs, 2H), 7.41 (m, 2H), 7.33 (m, 2H), 7.17 (m, 2H0, 6.87 (m, 1H), 6.74 (m, 1H), 6.49 (bs, 1H), 5.01 (m, 1H) 4.39 (m, 1H), 4.07 (m, 1H), 3.32 (m, 2H, 3.15 (m, 1H) 2.93 (bs, 3H), 2.69 (m, 2H), 1.83 (m, 2H). HPLC purity: 94.5%.

### Example 122

### 1-[6-Chloro-7-(2-methyl-2H-pyrazol-3-yl)-3,4-dihydro-2H-benzo[1,4]oxazine-2-carbonyl]-4-(4-fluoro-benzyl)-piperidine-4-carbonitrile

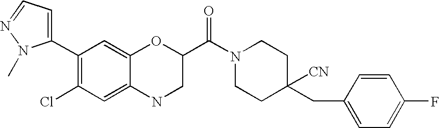

1-[6-Chloro-7-(2-methyl-2H-pyrazol-3-yl)-3,4-dihydro-2H-benzo[1,4]oxazine-2-carbonyl]-4-(4-fluoro-benzyl)-piperidine-4-carbonitrile was prepared by similar process as described in example 120. LC/MS [M+H]⁺: 494.2. ¹H-NMR (400 MHz, DMSO-d₆) δ (ppm): 7.43 (bs, 1H), 7.33 (m, 2H), 7.18 (m, 2H), 6.75 (m, 2H), 6.49 (bs, 1H), 6.21 (bs, 1H), 5.00 (bs, 1H), 4.39 (m, 1H), 3.62 (s, 3H), 3.44 (m, 1H), 3.11 (m, 1H), 2.93 (s, 2H), 1.71 (m, 4H). HPLC purity: 88.5%.

### Example 123

### 1-[6-Chloro-7-(1-methyl-1H-pyrazol-4-yl)-3,4-dihydro-2H-benzo[1,4]oxazine-2-carbonyl]-4-(4-fluoro-benzyl)-piperidine-4-carbonitrile

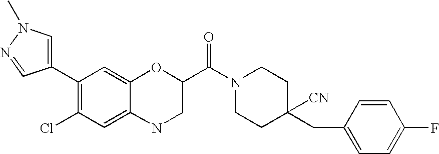

1-[6-Chloro-7-(1-methyl-1H-pyrazol-4-yl)-3,4-dihydro-2H-benzo[1,4]oxazine-2-carbonyl]-4-(4-fluoro-benzyl)-piperidine-4-carbonitrile was prepared by similar process as described in example 120. LC/MS [M+H]⁺: 494.4. ¹H-NMR (400 MHz, DMSO-d₆) δ (ppm): 7.94-6.16(m, 9H), 4.97(m, 1H), 4.39(m, 1H), 4.09(m, 1H), 3.85(s, 3H), 3.41-1.55(m, 10H). HPLC purity: 87.1%.

### Example 124

### Laevo and dextro enantiomers of 1-(6-Chloro-7-dimethylamino-3,4-dihydro-2H-benzo[1,4]oxazine-2-carbonyl)-4-(4fluoro-benzyl)-piperidine-4-carbonitrile

### Preparation of two epimers of 6-Chloro-7-dimethylamino-2-(2-hydroxy-1-phenyl-ethylcarbamoyl)-2,3-dihydro-benzo[1,4]oxazine-4-carboxylic acid tert-butyl ester

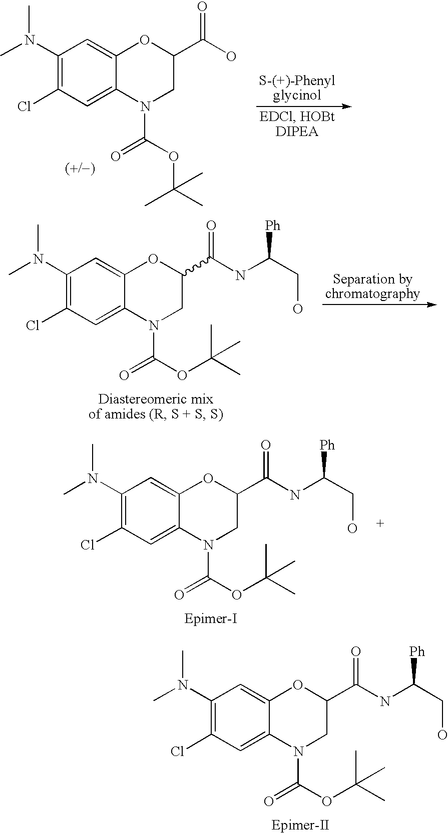

Racemic-6-Chloro-7-dimethylamino-2,3-dihydro-benzo[1,4]oxazine-2,4-dicarboxylic acid 4-tert-butyl ester was prepared as described in example 51.

To a solution of 6-Chloro-7-dimethylamino-2,3-dihydro-benzo[1,4]oxazine-2,4-dicarboxylic acid 4-tert-butyl ester (13.5 g, 0.038 mol) in DCM was added (S)-(+)-phenylglycinol (5.19 g, 0.038 mmol), EDCI (9.46 g, 0.049 mol), HOBt (2.56 g, 0.019 mol) and DIPEA (19.3 ml, 0.12 mol) at 5-10° C. The reaction mixture was stirred at room temperature for 15 hours then diluted with water and extracted with DCM. The organic layer was concentrated in vacuo and subjected to column chromatography (Si-gel, 25-35% ethyl acetate-hexane) to afford epimer-I (4.5 g , 24.9%) and epimer-II (˜4 g) of 6-Chloro-7-dimethylamino-2-(2-hydroxy-1-phenyl-ethylcarbamoyl)-2,3-dihydro-benzo[1,4]oxazine-4-carboxylic acid tert-butyl ester. LC/MS [M+H]⁺: 476.4

### Example 124a

### Laevo-isomer of 1-(6-Chloro-7-dimethylamino-3,4-dihydro-2H-benzo[1,4]oxazine-2-carbonyl)-4-(4-fluoro-benzyl)-piperidine-4-carbonitrile

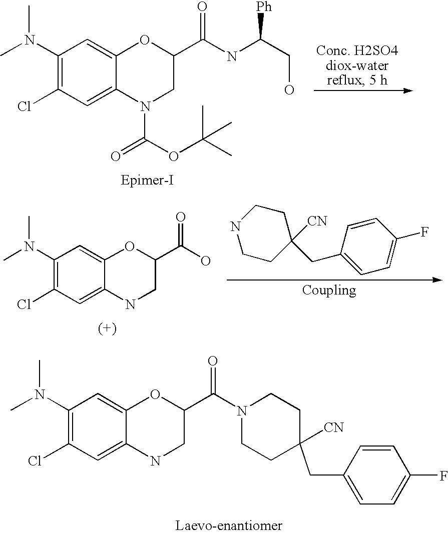

### Step-1

Epimer- I of 6-Chloro-7-dimethylamino-2-(2-hydroxy-1-phenyl-ethylcarbamoyl)-2,3-dihydro-benzo[1,4]oxazine-4-carboxylic acid tert-butyl ester ([α]=−2.7°; c 1.03% in CHCl₃) 4.4 g, was dissolved in dioxane-water (1:1) and was added 3 (N) H₂SO₄ (50 ml) dropwise. The solution was refluxed for 5-6 hours and pH was adjusted to 8-9. The organics were extracted with ethyl acetate and 10% EtOH-ethyl acetate. The organic layer was washed with brine solution and dried over Na₂SO₄. The organic layer was concentrated to afford 1.5 g (63.5%) of the dextro-enantiomer of 6-Chloro-7-dimethylamino-3,4-dihydro-2H-benzo[1,4]oxazine-2-carboxylic acid. LC/MS [M+H]⁺: 257.1. [α]=+38°; c 0.094% in MeOH

### Step-2

The dextro-enantiomer of 6-Chloro-7-dimethylamino-3,4-dihydro-2H-benzo[1,4]oxazine-2-carboxylic acid ([α]=+38°; c 0.094% in MeOH) (1.4 g, 5.45 mmol) in dichloromethane were added EDCI (1.35 g, 7.08 mmol), HOBt (370 mg, 2.72 mmol), DIPEA (3 ml, 16.3 mmol) and 4-(4-Fluoro-benzyl)-piperidine-4-carbonitrile (1.55 g, 5.45 mmol) at 5° C. The reaction mixture was stirred at room temperature for 20 hours. The reaction mixture was diluted with water and extracted with dichloromethane. The organic layer was washed successively with water, brine and dried over Na₂SO₄ The organic solution was concentrated and purified over column chromatography (Si-gel, 35% ethyl acetate-hexane) to afford 1.1 g (44.8%) of laevo enantiomer of 1-(6-Chloro-7-dimethylamino-3,4-dihydro-2H-benzo[1,4]oxazine-2-carbonyl)-4-(4-fluoro-benzyl)-piperidine-4-carbonitrile. LC/MS [M+H]⁺: 457.2. ¹H-NMR (400 MHz, DMSO-d₆) δ (ppm): 7.34 (bs, 2H), 7.20 (m, 2H), 6.58 (m, 2H), 5.74 (bs, 1H), 4.94 (m, 1H), 4.37 (m, 1H), 4.09 (m, 1H), 3.22 (m, 2H), 2.95 (m, 2H), 2.70 (m, 1H), 2.49 (s, 6H), 1.81 (m, 4H), 1.54 (m, 1H).

Chiral HPLC: 99.4%; ee 98.7%. [α]=−46.1°; c 0.97% in CHCl₃.

### Example 124b

### Dextro-isomer of 1-(6-Chloro-7-dimethylamino-3,4-dihydro-2H-benzo[1,4]oxazine-2-carbonyl)-4-(4-fluoro-benzyl)-piperidine-4-carbonitrile

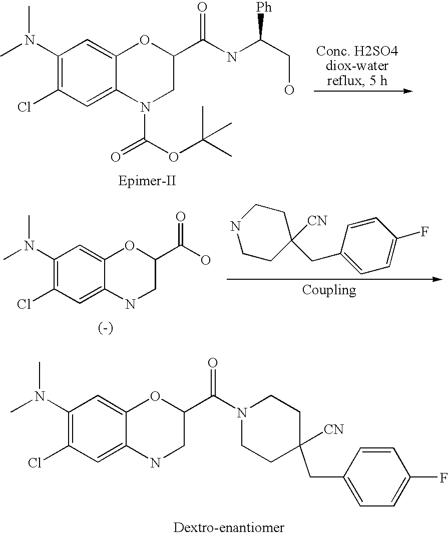

### Step-1

Epimer-II of 6-Chloro-7-dimethylamino-2-(2-hydroxy-1-phenyl-ethylcarbamoyl)-2,3-dihydro-benzo[1,4]oxazine-4-carboxylic acid tert-butyl ester (isolated in step-1) 500 mg, was dissolved in dioxane-water (1:1) and was added 3(N) H₂SO₄ 50 ml dropwise. The solution was refluxed for 5-6 hours and pH was adjusted to 8-9. The organics was extracted with ethyl acetate and 10% EtOH-ethyl acetate. The organic layer was washed with brine solution and dried over Na₂SO₄. The organic layer was concentrated to afford 352 mg of laevo- isomer of 6-Chloro-7-dimethylamino-3,4-dihydro-2H-benzo[1,4]oxazine-2-carboxylic acid.

LC/MS [M+H]⁺: 257.1.

### Step-2

The laevo-isomer of 6-Chloro-7-dimethylamino-3,4-dihydro-2H-benzo[1,4]oxazine-2-carboxylic acid (350 mg, 1.36 mmol) in dichloromethane were added EDCI (339.3 mg, 1.77 mmol), HOBt (91.9 mg, 0.68 mmol), DIPEA (0.7 ml, 4.1 mmol) and 4-(4-Fluoro-benzyl)-piperidine-4-carbonitrile (386.3 mg, 1.7 mmol) at 5° C. The reaction mixture was stirred at room temperature for 20 hours. The reaction mixture was diluted with water and extracted with dichloromethane. The organic layer was washed with water, brine solution and dried over Na₂SO₄ The organic solution was concentrated and purified over column chromatography (Si-gel, 35% ethyl acetate-hexane) to afford 62 mg (10%) of the dextro enantiomer of 1-(6-Chloro-7-dimethylamino-3,4-dihydro-2H-benzo[1,4]oxazine-2-carbonyl)-4-(4-fluoro-benzyl)-piperidine-4-carbonitrile. LC/MS [M+H]⁺: 457.2. ¹H-NMR (400 MHz, DMSO-d₆) δ (ppm): 7.34 (bs, 2H), 7.20 (m, 2H), 6.58 (m, 2H), 5.74 (bs, 1H), 4.94 (m, 1H), 4.37 (m, 1H), 4.09 (m, 1H), 3.22 (m, 2H), 2.95 (m, 2H), 2.70 (m, 1H), 2.49 (s, 6H), 1.81 (m, 4H), 1.54 (m, 1H). Chiral HPLC: 100%; ee=100%. [α]=+50.1°; c 0.63% in CHCl₃.

### Example 125

### 1-(6-Chloro-7-pyrazol-1-yl-3,4-dihydro-2H-benzo[1,4]oxazine-2-carbonyl)-4-(4-fluoro-benzyl)-piperidine-4-carbonitrile

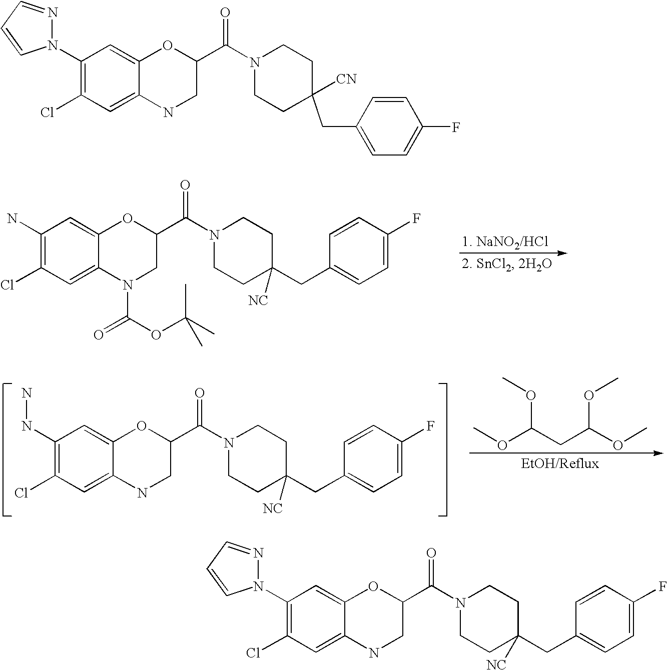

To a solution of 7-Amino-6-chloro-2-[4-cyano-4-(4-fluoro-benzyl)-piperidine-1-carbonyl]-2,3-dihydro-benzo[1,4]oxazine-4-carboxylic acid tert-butyl ester (250 mg, 0.47 mmol) in aqueous HCl (5 ml) was added aqueous NaNO₂ solution (64.9 mg, 0.94 mmol) at −5° C. and small amount of 1,4-dioxane was added to dissolve the compound completely and then stirred at 5-10° C. for 2 hour. Then SnCl₂.2H₂O(446 mg, 2.35 mmol) in concentrated HCl was added and colour of the reaction mass became off white from light yellow with precipitation. The reaction mixture was stirred at 10° C. for 1 hour. The solid was filtered and subjected to forward reaction without purification. To a solution of the crude mass of 1-(6-Chloro-7-hydrazino-3,4-dihydro-2H-benzo[1,4]oxazine-2-carbonyl)-4-(4-fluoro-benzyl)-piperidine-4-carbonitrilein ethanol (5 ml) was added tetramethoxy propane (0.1 ml, 0.61 mmol) and refluxed for 1 hour. A yellow solid was precipitated on cooling. The yellow solid was filtered, washed with cold ethanol. To that added water, basified with 1(N) NaOH solution, extracted with ethyl acetate, washed with water, brine, dried over Na₂SO₄ and then concentrated. The organic mass was purified over column chromatography (silica gel, 0.8% methanol-DCM) to afford 20 mg (8.9%) of 1-(6-Chloro-7-pyrazol-1-yl-3,4-dihydro-2H-benzo[1,4]oxazine-2-carbonyl)-4-(4-fluoro-benzyl)-piperidine-4-carbonitrile. LC/MS [M+H]⁺: 480.4. ¹H-NMR (400 MHz, DMSO-d₆) δ (ppm): 9.36 (m, 1H), 8.21 (m, 1H), 7.67 (m, 1H), 7.33 (m, 2H), 7.19 (m, 2H), 7.02 (m, 2H), 5.64 (m, 1H), 5.35 (m, 1H), 4.32-1.74 (m, 11H). HPLC purity: 84.8%.

### Example 126

### 1-(6-Chloro-7-pyrimidin-5-yl-3,4-dihydro-2H-benzo[1,4]oxazine-2-carbonyl)-4-(4-fluoro-benzyl)-piperidine-4-carbonitrile

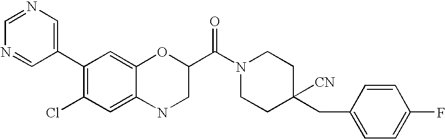

1-(6-Chloro-7-pyrimidin-5-yl-3,4-dihydro-2H-benzo[1,4]oxazine-2-carbonyl)-4-(4-fluoro-benzyl)-piperidine-4-carbonitrile was prepared by similar process as described in example 120. LC/MS [M+H]⁺: 492.4. LCMS purity: 90.7%.

### Example 127

### 1-(6-Chloro-7-hexa deuterio dimethylamino-3,4-dihydro-2H-benzo[1,4]oxazine-2-carbonyl)-4-(4-fluoro-benzyl)-piperidine-4-carbonitrile

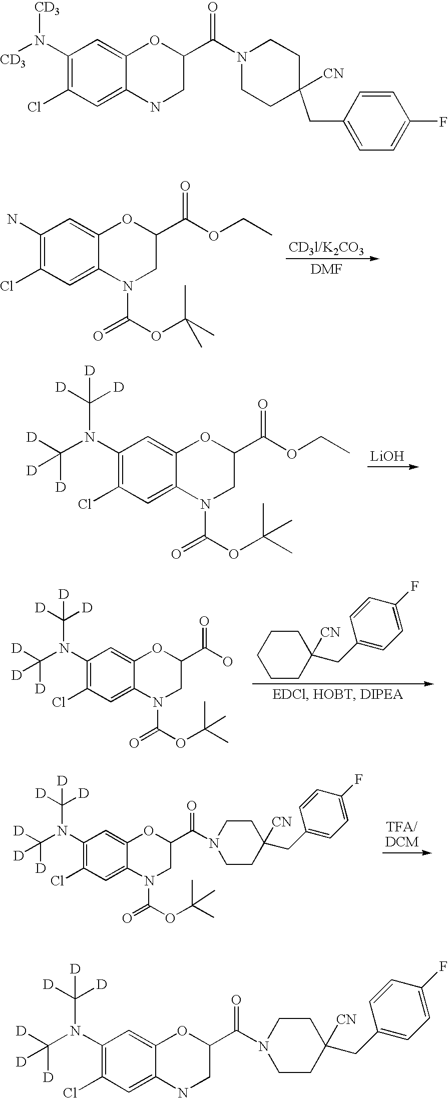

7-Amino-6-chloro-2,3-dihydro-benzo[1,4]oxazine-2,4-dicarboxylic acid 4-tert-butyl ester 2-ethyl ester was prepared by a process similar to that described in example 51.
Step-1: 6-Chloro-7-hexa deuterio dimethylamino-2,3-dihydro-benzo[1,4]oxazine-2,4-dicarboxylic acid 4-tert-butyl ester 2-ethyl ester

7-Amino-6-chloro-2,3-dihydro-benzo[1,4]oxazine-2,4-dicarboxylic acid 4-tert-butyl ester 2-ethyl ester (400 mg, 1.12 mmol) was dissolved in DMF. Then to it K₂CO₃ (464 mg, 3.36 mmol) and CD₃I (0.21 ml, 3.36 mmol) were added at cold condition. It was then heated at 40° C.-45° C. for overnight. Then it was diluted with water and extracted with ethyl acetate. Organic layer was washed with water and brine. It was dried and concentrated to produce 6-Chloro-7-hexa deuterio dimethylamino-2,3-dihydro-benzo[1,4]oxazine-2,4-dicarboxylic acid 4-tert-butyl ester 2-ethyl ester (250 mg, 57.1%).

LCMS: 391.2.

Step-2:6-Chloro-7-hexa deuterio dimethylamino-2,3-dihydro-benzo[1,4]oxazine-2,4-dicarboxylic acid 4-tert-butyl ester

6-Chloro-7-hexa deuterio dimethylamino-2,3-dihydro-benzo[1,4]oxazine-2,4-dicarboxylic acid 4-tert-butyl ester 2-ethyl ester (250 mg, 0.64 mmol) was dissolved in (4:1) THF: water and to it LiOH (55 mg, 1.3 mmol) was added at cold condition. It was stirred at RT for 3 hours. Then it was concentrated and neutralized by 50% aq. glacial acetic acid. It was extracted with ethyl acetate and organic layer was washed with water, brine. It was dried, concentrated and washed with hexane to produce 6-Chloro-7-hexa deuterio dimethylamino-2,3-dihydro-benzo[1,4]oxazine-2,4-dicarboxylic acid 4-tert-butyl ester (180 mg, 77.5%). LCMS: 363.4.

Step-3: 6-Chloro-2-[4-cyano-4-(4-fluoro-benzyl)-piperidine-1-carbonyl]-7-hexa deuteriodimethylamino-2,3-dihydro-benzo[1,4]oxazine-4-carboxylic acid tert-butyl ester

6-Chloro-7-hexa deuterio dimethylamino-2,3-dihydro-benzo[1,4]oxazine-2,4-dicarboxylic acid 4-tert-butyl ester (180 mg, 0.49 mmol) was dissolved in dry DMF and to it DIPEA (0.3 ml, 1.48 mmol) was added drop wise at cold condition. EDCI (143 mg, 0.74 mmol), HOBT (108 mg, 0.49 mmol) were then added to the reaction mixture and stirred for 0.5 hour. 4-(4-Fluoro-benzyl)-piperidine-4-carbonitrile (109 mg, 0.49 mmol) was then added to the reaction mixture and stirred overnight. It was then diluted with water and extracted with ethyl acetate. Organic layer was washed with water and brine. Finally it was dried, concentrated and purified through column chromatography using (10-30%) EA/hexane in SiO₂ to produce 6-Chloro-2-[4-cyano-4-(4-fluoro-benzyl)-piperidine-1-carbonyl]-7-hexa deuteriodimethylamino-2,3-dihydro-benzo[1,4]oxazine-4-carboxylic acid tert-butyl ester (185 mg, 67.04%). LCMS: 563.2.

Step-4: 1-(6-Chloro-7-hexa deuterio dimethylamino-3,4-dihydro-2H-benzo[1,4]oxazine-2-carbonyl)-4-(4-fluoro-benzyl)-piperidine-4-carbonitrile

6-Chloro-2-[4-cyano-4-(4-fluoro-benzyl)-piperidine-1-carbonyl]-7-hexa deuteriodimethylamino-2,3-dihydro-benzo[1,4]oxazine-4-carboxylic acid tert-butyl ester (180 mg, 0.32 mmol) was dissolved in DCM. TFA (0.25 ml, 3.2 mmol) was added to the reaction mixture at cold condition. It was then stirred at RT for 1 hour and concentrated. It was diluted with water and basified. After that it was extracted with ethyl acetate. Organic layer was washed with water, brine and dried. It was concentrated and washed with hexane to produce 1-(6-Chloro-7-hexa deuterio dimethylamino-3,4-dihydro-2H-benzo[1,4]oxazine-2-carbonyl)-4-(4-fluoro-benzyl)-piperidine-4-carbonitrile (140 mg, 94.5%). LC/MS [M+H]⁺: 463.4. ¹H-NMR (400 MHz, DMSO-d₆) δ (ppm): 7.34(brs, 2H), 7.19(m, 2H), 6.58(m, 2H), 5.75(s, 1H), 4.96-1.23(m, 13H).

HPLC purity: 92.6%

### Example 128

### 1-(6-Chloro-7-furan-2-yl-3,4-dihydro-2H-benzo[1,4]oxazine-2-carbonyl)-4-(4-fluoro-benzyl)-piperidine-4-carbonitrile

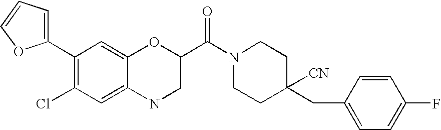

1-(6-Chloro-7-furan-2-yl-3,4-dihydro-2H-benzo[1,4]oxazine-2-carbonyl)-4-(4-fluoro-benzyl)-piperidine-4-carbonitrile was prepared by similar process as described in example 120. LC/MS [M+H]⁺: 480.2. ¹H-NMR (400 MHz, DMSO-d₆) δ (ppm): 7.71-6.44(m, 10H), 5.00(m, 1H), 4.39(m, 1H), 4.11(m, 1H), 3.40-1.53(m, 10H). HPLC purity: 91.2%.

### Example 129

### 1-(6-Chloro-7-furan-3-yl-3,4-dihydro-2H-benzo[1,4]oxazine-2-carbonyl)-4-(4-fluoro-benzyl)-piperidine-4-carbonitrile

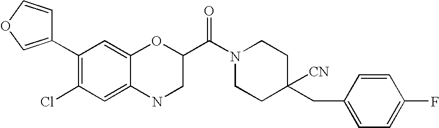

1-(6-Chloro-7-furan-3-yl-3,4-dihydro-2H-benzo[1,4]oxazine-2-carbonyl)-4-(4-fluoro-benzyl)-piperidine-4-carbonitrile was prepared by similar process as described in example 120. LC/MS [M+H]⁺: 480.2. ¹H-NMR (400 MHz, DMSO-d₆) δ (ppm): 7.94 (s, 1H), 7.70 (m, 1H), 7.35 (m, 2H), 7.19 (m, 2H), 6.90 (m, 1H), 6.78 (m, 1H), 6.68 (s, 1H), 6.27 (bs, 1H), 4.99 (m, 1H), 4.41 (m, 1H0, 4.10 (m, 1H), 3.40 (m, 1H), 2.95 (bs, 2H), 2.68 (m, 1H), 1.81 (m, 4H), 1.54 (m, 2H). HPLC purity: 90.1%.

### Example 130

### 1-[6-Chloro-7-(4-methyl-imidazol-1-yl)-3,4-dihydro-2H-benzo[1,4]oxazine-2-carbonyl]-4-(4-fluoro-benzyl)-piperidine-4-carbonitrile

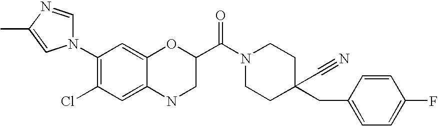

1-[6-Chloro-7-(4-methyl-imidazol-1-yl)-3,4-dihydro-2H-benzo[1,4]oxazine-2-carbonyl]-4-(4-fluoro-benzyl)-piperidine-4-carbonitrile was prepared by a process similar to that described in example 106. LC/MS [M+H]⁺: 494.2. ¹H-NMR (400 MHz, DMSO-d₆) δ (ppm): 7.57 (bs, 1H), 7.33 (m, 2H), 7.18 (m, 2H), 6.95 (m, 1H), 6.85 (m, 1H), 6.75 (bs, 1H), 6.46 (bs, 1H), 5.03 (m, 1H), 4.38 (m, 1H), 3.45 (m, 1H), 3.18 (m, 1H), 2.93 (s, 3H), 2.73 (m, 2H), 2.13 (s, 3H), 1.90 (m, 2H), 1.74 (m, 2H). HPLC purity: 95.5%.

### Example 131

### 1-[6-Chloro-7-(3-methyl-pyrazol-1-yl)-3,4-dihydro-2H-benzo[1,4]oxazine-2-carbonyl]-4-(4-fluoro-benzyl)-piperidine-4-carbonitrile

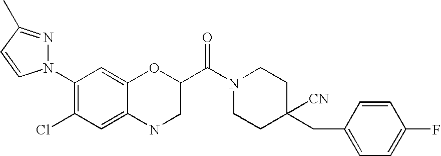

1-[6-Chloro-7-(3-methyl-pyrazol-1-yl)-3,4-dihydro-2H-benzo[1,4]oxazine-2-carbonyl]-4-(4-fluoro-benzyl)-piperidine-4-carbonitrile was prepared by a process similar to that described in example 106. LC/MS [M+H]⁺: 494.2. ¹H-NMR (400 MHz, DMSO-d₆) δ (ppm): 7.75 (bs, 1H), 7.34 (m, 2H), 7.19 (m, 2H), 6.86 (m, 1H), 6.72 (bs, 1H), 6.44 (bs, 1H), 6.21 (bs, 1H), 5.03 (m, 1H), 4.39 (m, 1H), 4.10 (m, 1H), 3.43 (m, 2H), 3.17 (m, 1H), 2.94 (s, 3H), 2.69 (m, 2H), 2.21 (s, 3H), 1.83-1.53 (m, 2H). HPLC purity: 92.6%.

### Example 132

### 1-(6-Chloro-7-[1,2,4]triazol-4-yl-3,4-dihydro-2H-benzo[1,4]oxazine-2-carbonyl)-4-(4-fluoro-benzyl)-piperidine-4-carbonitrile

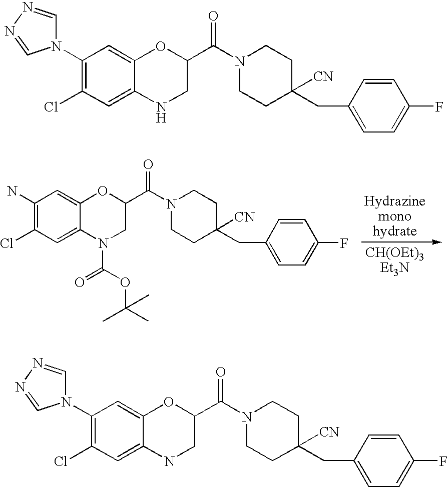

A mixture of 7-Amino-6-chloro-2-[4-cyano-4-(4-fluoro-benzyl)-piperidine-1-carbonyl]-2,3-dihydro-benzo[1,4]oxazine-4-carboxylic acid tert-butyl ester (400 mg, 0.67 mmol), Hydrazine hydrate (84 mg, 1.5 mmol) and triethylamine (3 ml) was refluxed in triethylothoformate at 150° C. overnight fitted with Dean-Stark apparatus. The triethylorthoformate was ditilled off and the reaction mixture was diluted with water and extracted with ethyl acetate. The organic layer was concentrated and purified over column chromatography (Si-gel, 1% MeoH-DCM) to afford 20 mg (6.1%) of 1-(6-Chloro-7-[1,2,4]triazol-4-yl-3,4-dihydro-2H-benzo[1,4]oxazine-2-carbonyl)-4-(4-fluoro-benzyl)-piperidine-4-carbonitrile. LC/MS [M+H]⁺: 481.4. ¹H-NMR (400 MHz, DMSO- d₆) δ (ppm): 8.68 (bs, 2H), 7.33 (bs, 2H), 7.18 (m, 2H), 7.05 (m, 1H), 6.79 (bs, 1H), 6.61 (bs, 1H), 5.07 (bs, 1H), 4.38 (bs, 1H), 3.47-1.51 (m, 10H). HPLC purity: 93.0%

### Example 133

### 1-(6-Chloro-7-[1,2,3]triazol-1-yl-3,4-dihydro-2H-benzol[1,4]oxazine-2-carbonyl)-4-(4-fluoro-benzyl)-piperidine-4-carbonitrile

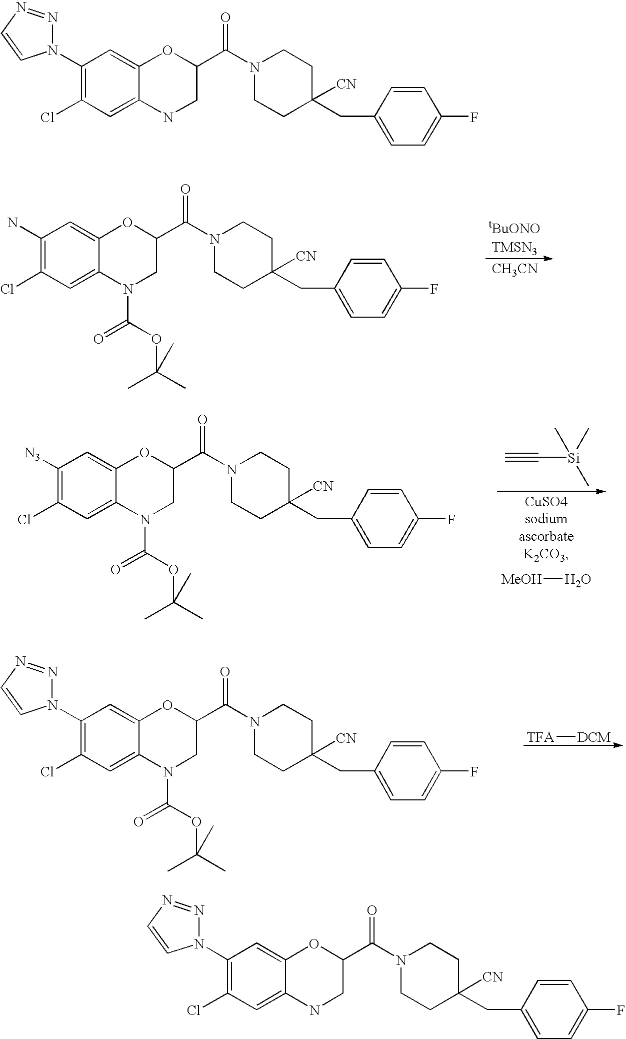

Step-1: 7-Azido-6-chloro-2-[4-cyano-4-(4-fluoro-benzyl)-piperidine-1-carbonyl]-2,3-dihydro-benzo[1,4]oxazine-4-carboxylic acid tert-butyl ester

To a solution of 7-Amino-6-chloro-2-[4-cyano-4-(4-fluoro-benzyl)-piperidine-1-carbonyl]-2,3-dihydro-benzo[1,4]oxazine-4-carboxylic acid tert-butyl ester (200 mg, 0.38 mmol) in acetonitrile was added slowly tert-butyl nitrite (40.05 ml, 0.42 mmol) followed by TMS azide (0.09 ml, 0.76 mmol) dropwise at 0° C. The resulting solution was stirred at room temperature for one hour. The reaction mixture was concentrated and ourified over column chromatography (Si-gel, 0.2% MEOH-DCM) to afford 150 mg (71%) of 7-Azido-6-chloro-2-[4-cyano-4-(4-fluoro-benzyl)-piperidine-1-carbonyl]-2,3-dihydro-benzo[1,4]oxazine-4-carboxylic acid tert-butyl ester. The material was used in next step without any characterization. LC/MS [M+H+NH₃]⁺: 572.5, 574.3

Step-2: 6-Chloro-2-[4-cyano-4-(4-fluoro-benzyl)-piperidine-1-carbonyl]-7-[1,2,3]triazol-1-yl-2,3-dihydro-benzo[1,4]oxazine-4-carboxylic acid tert-butyl ester

To a solution of 7-Azido-6-chloro-2-[4-cyano-4-(4-fluoro-benzyl)-piperidine-1-carbonyl]-2,3-dihydro-benzo[1,4]oxazine-4-carboxylic acid tert-butyl ester (150 mg, 0.27 mmol), potassium carbonate (373.6 mg, 2.7 mmol), CuSO₄ (13.5 mg, 0.054 mmol), sodium ascorbate (21.4 mg, 0.11 mmol) and trimethylsilylacetylene (0.05 ml, 0.32 mmol) in methanol-dioxane-water (1:1:1) in sealed tube. The reaction mixture was heated at 100° C. overnight. The resulting suspension was extracted with ethyl acetate. The organic layer was washed with water and dried over sodium sulfate. The organics was purified over column chromatography (Si-gel, 0.5% MeOH-DCM) to afford 60 mg (46.2%) of 6-Chloro-2-[4-cyano-4-(4-fluoro-benzyl)-piperidine-1-carbonyl]-7-[1,2,3]triazol-1-yl-2,3-dihydro-benzo[1,4]oxazine-4-carboxylic acid tert-butyl ester.

LC/MS [M-FH]⁺: 581.4.

Step-3: 1-(6-Chloro-7-[1,2,3]triazol-1-yl-3,4-dihydro-2H-benzo[1,4]oxazine-2-carbonyl)-4-(4-fluoro-benzyl)-piperidine-4-carbonitrile

To a solution of 6-Chloro-2-[4-cyano-4-(4-fluoro-benzyl)-piperidine-1-carbonyl]-7-[1,2,3]triazol-1-yl-2,3-dihydro-benzo[1,4]oxazine-4-carboxylic acid tert-butyl ester (60 mg, 0.103 mmol)) in DCM was added trifluoroacetic acid (0.3 ml). The reaction mixture was stirred for about 5 h then concentrated and washed with dry ether. The resulting TFA salt was diluted with a minimum volume of water and neutralized with aq. NaOH solution at 5-10° C. Extraction of the neutralized solution with ethyl acetate followed by concentration afforded 30 mg (60.5%) of 1-(6-Chloro-7-[1,2,3]triazol-1-yl-3,4-dihydro-2H-benzo[1,4]oxazine-2-carbonyl)-4-(4-fluoro-benzyl)-piperidine-4-carbonitrile. LC/MS [M+H]⁺: 481.2. ¹H-NMR (400 MHz, DMSO-d₆) δ (ppm): 8.36 (m, 1H), 7.89 (m, 1H), 7.33 (m, 2H), 7.18 (m, 2H), 7.00 (m, 1H), 6.80 (s, 1H), 6.68 (s, 1H), 5.07 (m, 1H), 4.38 (m, 1H), 4.06 (m, 1H), 3.46 (m, 1H), 3.40 (m, 1H), 3.19 (m, 1H), 2.93 (s, 2H), 2.69 (m, 1H), 1.80 (m, 2H), 1.72 (m, 1H), 1.54 (m, 1H). HPLC purity: 95.5%.

### Example 134

### 1-(6-Chloro-4-methyl-7-oxazol-5-yl-3,4-dihydro-2H-benzo[1,4]oxazine-2-carbonyl)-4-(4-fluoro-benzyl)-piperidine-4-carbonitrile

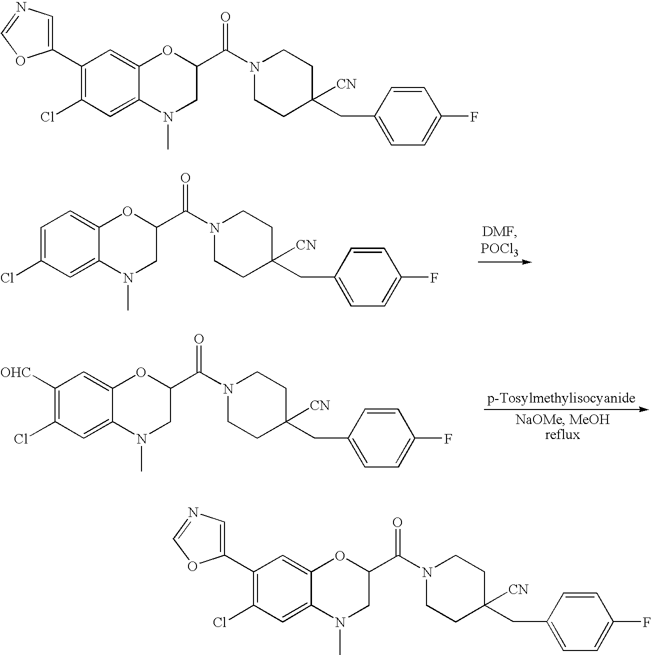

Step-1: 1-(6-Chloro-7-formyl-3,4-dihydro-2H-benzo[1,4]oxazine-2-carbonyl)-4-(4-fluoro-benzyl)-piperidine-4-carbonitrile

To a dry DMF (0.6 ml0 was added POCl₃ (0.6 ml) and sirirred the solution for 30 min at room temperature. To the DMF-POCl₃ solution was added a solution of 1-(6-Chloro-3,4-dihydro-2H-benzo[1,4]oxazine-2-carbonyl)-4-(4-fluoro-benzyl)-piperidine-4-carbonitrile (880 mg, 2.06 mmol) in 2 ml DMF and stirred the solution at 60° C. overnight. The Reaction mixture was diluted with ice and extracted with ethyl acetate. The organic layer was washed with water and dried over sodium sulfate. The organic layer was concentrated under redused pressure to afford 750 mg (79.9%) of 1-(6-Chloro-7-formyl-3,4-dihydro-2H-benzo[1,4]oxazine-2-carbonyl)-4-(4-fluoro-benzyl)-piperidine-4-carbonitrile. LC/MS [M+H]⁺: 456.0

Step-2: 1-(6-Chloro-4-methyl-7-oxazol-5-yl-3,4-dihydro-2H-benzo[1,4]oxazine-2-carbonyl)-4-(4-fluoro-benzyl)-piperidine-4-carbonitrile

To a mixture of 1-(6-Chloro-7-formyl-3,4-dihydro-2H-benzo[1,4]oxazine-2-carbonyl)-4-(4-fluoro-benzyl)-piperidine-4-carbonitrile (130 mg, 0.29 mmol) and sodium methoxe (48.6 mg, 0.9 mmol) in methanol was added p-Tosylmethylisocyanide (68 mg, 0.35 mmol). The reaction mixture was refluxed one hour. The reaction mixture was concentrated, diluted with water and extracted with ethyl acetate. The organic layer was concentrated to afford 53 mg (36.9%) of 1-(6-Chloro-4-methyl-7-oxazol-5-yl-3,4-dihydro-2H-benzo[1,4]oxazine-2-carbonyl)-4-(4-fluoro-benzyl)-piperidine-4-carbonitrile

LC/MS [M+H]⁺: 495.0. ¹H-NMR (400 MHz, DMSO-d₆) δ (ppm): 8.40 (m, 1H), 7.53 (bs, 1H), 7.34 (bs, 2H), 7.18 (m, 2H), 7.11 (m, 1H), 6.82 (m, 1H), 5.19 (bs, 1H), 4.37 (m, 1H), 4.12 (m, 1H), 3.41 (m, 2H), 3.20 (m, 1H), 2.93 (bs, 4H), 2.67 (m, 1H), 1.91-1.53 (m, 5H). HPLC purity: 90.7%.

### Example 135

### 1-[6-Chloro-7-dimethylamino-4-(1H-[1,2,4]triazol-3-yl)-3,4-dihydro-2H-benzo[1,4]oxazine-2-carbonyl]-4-(4-fluoro-benzyl)-piperidine-4-carbonitrile

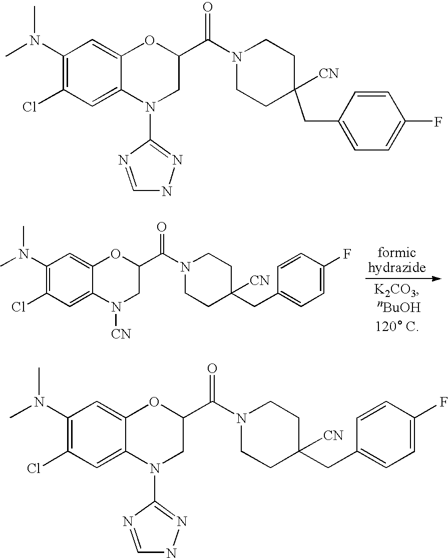

Preparation of 6-Chloro-2-[4-cyano-4-(4-fluoro-benzyl)-piperidine-1-carbonyl]-7-dimethylamino-2,3-dihydro-benzo[1,4]oxazine-4-carbonitrile was described in example 76.
Step-1: 1-[6-Chloro-7-dimethylamino-4-(1H-[1,2,4]triazol-3-yl)-3,4-dihydro-2H-benzo[1,4]oxazine-2-carbonyl]-4-(4-fluoro-benzyl)-piperidine-4-carbonitrile

To a solution of 6-Chloro-2-[4-cyano-4-(4-fluoro-benzyl)-piperidine-1-carbonyl]-7-dimethylamino-2,3-dihydro-benzo[1,4]oxazine-4-carbonitrile (100 mg, 0.21 mmol) in n-butanol was added formic hydrazide (16 mg, 0.21 mmol) and anhd. potassium carbonate (57 mg, 0.41 mmol). The reaction mixture was heated at 120° C. overnight. The reaction mixture was concentrated, diluted with water and extracted with ethyl acetate. The organic layer was washed with water and dried over sodium sulfate. The organic layer was concentrated and purified over column chromatography (Si-gel, 1.5% MeOH-DCM) to afford 9 mg (8%) of 1-[6-Chloro-7-dimethylamino-4-(1H-[1,2,4]triazol-3-yl)-3,4-dihydro-2H-benzo[1,4]oxazine-2-carbonyl]-4-(4-fluoro-benzyl)-piperidine-4-carbonitrile. LC/MS [M+H]⁺: 524.2. ¹H-NMR (400 MHz, DMSO-d₆) δ (ppm): 8.40 (m, 1H), 8.28 (m, 1H), 7.34 (m, 2H), 7.19 (m, 2H), 6.70 (m, 1H), 5.23 (m, 1H), 4.32 (m, 1H), 4.16 (m, 1H), 3.04 (m, 3H), 2.65 (s, 6H), 1.77 (m, 3H), 1.39 (m, 3H), 1.23 (m, 1H). HPLC purity: 87.0%.

### Example 136

### 1-[6-Chloro-7-(6-fluoro-pyridin-3-yl)-3,4-dihydro-2H-benzo[1,4]oxazine-2-carbonyl]-4-(4-fluoro-benzyl)-piperidine-4-carbonitrile

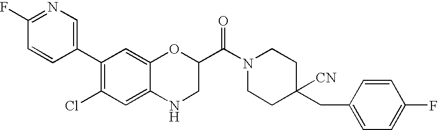

1-[6-Chloro-7-(6-fluoro-pyridin-3-yl)-3,4-dihydro-2H-benzo[1,4]oxazine-2-carbonyl]-4-(4-fluoro-benzyl)-piperidine-4-carbonitrilewas prepared by similar process as described in example 120.

LC/MS [M+H]⁺: 509.4. ¹H-NMR (400 MHz, DMSO-d₆) δ (ppm): 8.20 (bs, 1H), 7.98 (m, 1H), 7.33 (m, 2H), 7.17 (m, 3H), 6.85 (m, 1H), 6.75 (bs, 1H), 6.41 (bs, 1H), 5.00 (bs, 1H), 4.33 (m, 1H), 4.10 (m, 1H), 3.30 (m, 1H), 2.93 (s, 3H), 2.66 (m, 1H), 1.80 (m, 3H). HPLC purity: 92.9.

### Example 137

### 1-[6-Chloro-7-(6-methoxy-pyridin-3-yl)-3,4-dihydro-2H-benzo[1,4]oxazine-2-carbonyl]-4-(4-fluoro-benzyl)-piperidine-4-carbonitrile

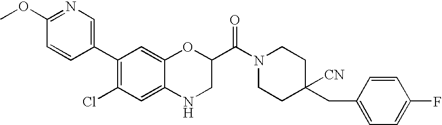

1-[6-Chloro-7-(6-methoxy-pyridin-3-yl)-3,4-dihydro-2H-benzo[1,4]oxazine-2-carbonyl]-4-(4-fluoro-benzyl)-piperidine-4-carbonitrile was prepared by similar process as described in example 120. LC/MS [M+H]⁺: 521.2. ¹H-NMR (400 MHz, DMSO-d₆) δ (ppm): 8.13 (s, 1H), 7.70 (d, 1H), 7.33 (m, 2H), 7.17 (m, 2H), 6.76 (m, 3H), 6.31 (bs, 1H), 4.98 (m, 1H), 4.39 (bs, 1H), 4.03 (m, 1H), 3.87 (s, 3H0, 3.42 (m, 1H), 3.15 (m, 1H), 2.93 (s, 3H), 2.72 (m, 2H), 1.80 (m, 4H). HPLC purity: 93.5%.

### Example 138

### 1-[6-Chloro-7-(2-methoxy-pyridin-3-yl)-3,4-dihydro-2H-benzo[1,4]oxazine-2-carbonyl]-4-(4-fluoro-benzyl)-piperidine-4-carbonitrile

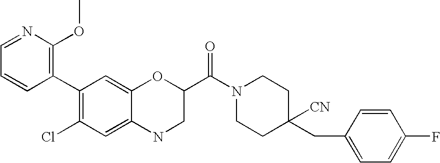

1-[6-Chloro-7-(2-methoxy-pyridin-3-yl)-3,4-dihydro-2H-benzo[1,4]oxazine-2-carbonyl]-4-(4-fluoro-benzyl)-piperidine-4-carbonitrile was prepared by similar process as described in example 120. LC/MS [M+H]⁺: 521.2. ¹H-NMR (400 MHz, DMSO-d₆) δ (ppm): 8.15 (bs, 1H), 7.51 (bs, 1H), 7.32 (m, 2H), 7.18 (m, 2H), 7.03 (bs, 1H), 6.69 (m, 2H), 6.26 (bs, 1H), 4.97 (bs, 1H), 4.39 (m, 1H), 4.05 (m, 1H), 3.81 (s, 3H), 3.42 (m, 1H), 3.18 (m, 2H), 2.93 (s, 3H), 2.69 (m, 1H), 1.54 (m, 4H). HPLC purity: 93.9%.

### Example 139

### 1-(6-Chloro-7-hydroxymethyl-3,4-dihydro-2H-benzo[1,4]oxazine-2-carbonyl)-4-(4-fluoro-benzyl)-piperidine-4-carbonitrile

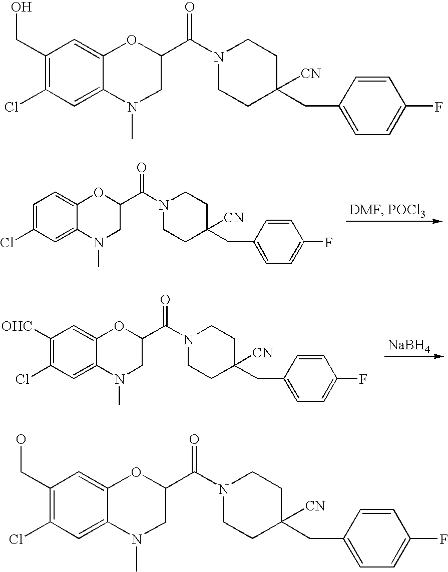

Step-1: 1-(6-Chloro-7-formyl-3,4-dihydro-2H-benzo[1,4]oxazine-2-carbonyl)-4-(4-fluoro-benzyl)-piperidine-4-carbonitrile

To a dry DMF (0.6 ml0 was added POCl₃ (0.6 ml) and sirirred the solution for 30 min at room temperature. To the DMF-POCl₃ solution was added a solution of 1-(6-Chloro-3,4-dihydro-2H-benzo[1,4]oxazine-2-carbonyl)-4-(4-fluoro-benzyl)-piperidine-4-carbonitrile (880 mg, 2.06 mmol) in 2 ml DMF and stirred the solution at 60° C. overnight. The Reaction mixture was diluted with ice and extracted with ethyl acetate. The organic layer was washed with water and dried over sodium sulfate. The organic layer was concentrated under redused pressure to afford 750 mg (79.9%) of 1-(6-Chloro-7-formyl-3,4-dihydro-2H-benzo[1,4]oxazine-2-carbonyl)-4-(4-fluoro-benzyl)-piperidine-4-carbonitrile. LC/MS [M+H]⁺: 456.0.

Step-2: 1-(6-Chloro-7-hydroxymethyl-3,4-dihydro-2H-benzo[1,4]oxazine-2-carbonyl)-4-(4-fluoro-benzyl)-piperidine-4-carbonitrile

To a solution of 1-(6-Chloro-7-formyl-3,4-dihydro-2H-benzo[1,4]oxazine-2-carbonyl)-4-(4-fluoro-benzyl)-piperidine-4-carbonitrile (100 mg, 0.22 mmol) in methanol was added sodium borohydride (12 mg, 0.31 mmol) at 0° C. and stirred at 5-10° C. for 2 hours. The reaction mixture was quenched with ice and stirred for 10 mins. The reaction mixture aws concentrated and extracted with ethyl acetate. The organic layer was washed with water and dried over sodium sulfate. The organic layer was concentrated under redused pressure and purified over column chromatography (Si-gel, 0.5% MeOH-DCM) to afford 50 mg (49.6%) of 1-(6-Chloro-7-hydroxymethyl-3,4-dihydro-2H-benzo[1,4]oxazine-2-carbonyl)-4-(4-fluoro-benzyl)-piperidine-4-carbonitrile. LC/MS [M+H]⁺: 458.2. ¹H-NMR (400 MHz, DMSO-d₆) δ (ppm): 7.34 (bs, 2H), 7.19 (m, 1H), 6.85 (m, 1H), 6.67 (m, 1H), 5.14 (bs, 2H), 4.40 (bs, 2H, 4.12 (m, 1H), 3.23 (m, 3H0, 2.96 (bs, 2H), 2.83 (s, 3H), 2.67 (m, 1H), 1.78 (m, 3H), 1.54 (m, 1H). HPLC purity: 96.4%.

### Example 140

### 1-(6-Chloro-2-methyl-3,4-dihydro-2H-benzo[1,4]oxazine-2-carbonyl)-4-(4-fluoro-benzyl)-piperidine-4-carbonitrile

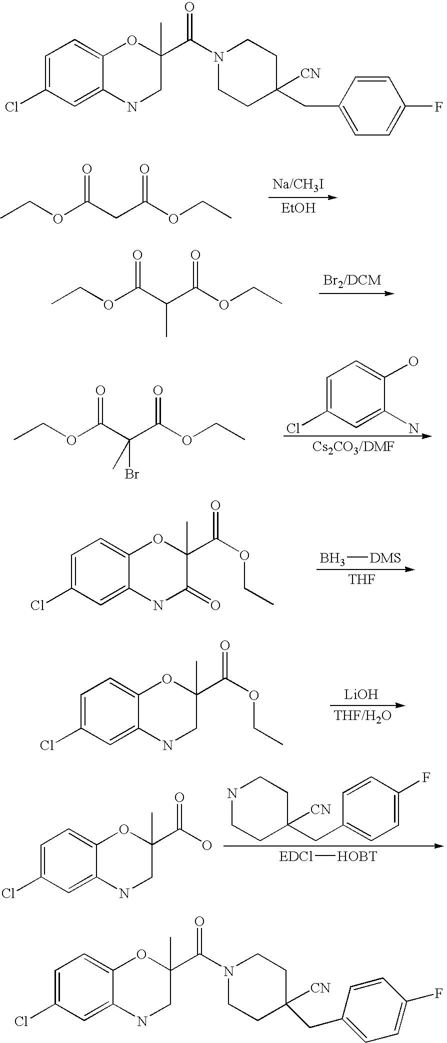

Step-1: 2-Methyl-malonic acid diethyl ester

Metallic sodium (790 mg, 34.3 mmol) was added to ethanol (70 ml) and stirred at room temperature for 1 hour. Diethyl malonate (4.8 ml, 31.2 mmol) was added drop wise to the previously prepared ethanolic solution at ice cold condition and stirred the solution at room temperature for 1 hour. Methyl iodide (5.9 ml, 93.6 mmol) was added drop wise to the ethanolic solution. The reaction mixture was stirred at room temperature overnight. The ethanolic solution was concentrated, diluted with water and extracted with ethyl acetate. The organic layer was washed with water, brine and dried over sodium sulphate. Concentration of the organic layer afforded 3.88 g (71.4%) of 2-Methyl-malonic acid diethyl ester. ¹H-NMR (400 MHz, DMSO-d₆) δ (ppm): 4.18 (m, 4H), 3.40 (m, 1H), 1.40 (d, 3H), 1.24 (q, 6H).

Step-2: 2-Bromo-2-methyl-malonic acid diethyl ester

To a stirred solution of 2-methyl-malonic acid diethyl ester (3.88 g, 22.3 mmol) in DCM (80 ml) was added a solution of Br₂ (1.14 ml, 22.3 mmol) in DCM (10 ml) drop wise under cold condition and the reaction mixture was stirred at room temperature for 1 hour. The reaction mixture was concentrated to afford 5.04 g (89.3%) of 2-bromo-2-methyl-malonic acid diethyl ester. GCMS [m/z]: 253.0

Step-3: 6-Chloro-2-methyl-3-oxo-3,4-dihydro-2H-benzo[1,4]oxazine-2-carboxylic acid ethyl ester

To a solution of 4-chloro-2-amino phenol (100 mg, 0.7 mmol) in DMF was added anhd. Cs₂CO₃ (570 mg, 1.8 mmol) and 2-bromo-2-methyl-malonic acid diethyl ester (264 mg, 1.04 mmol) drop wise under cold condition. The reaction mixture was stirred at room temperature overnight. The reaction mass was diluted with water, extracted with ethyl acetate, washed with water, brine and dried over Na₂SO₄. The organic layer was concentrated and the crude mass was purified by flash chromatography (silica gel, 10% ethlyl acetate-hexane) to afford 120 mg (63.5%) of 6-Chloro-2-methyl-3-oxo-3,4-dihydro-2H-benzo[1,4]oxazine-2-carboxylic acid ethyl ester. LC/MS [M+H]⁺: 270.2

Step-4: 6-Chloro-2-methyl-3,4-dihydro-2H-benzo[1,4]oxazine-2-carboxylic acid ethyl ester

To a stirred solution of 6-Chloro-2-methyl-3-oxo-3,4-dihydro-2H-benzo[1,4]oxazine-2-carboxylic acid ethyl ester (1.50 g, 5.7 mmol) in THF was added borane methyl sulfide (0.8 ml, 8.4 mmol) at 0° C. and then stirred at room temperature overnight. Methanol was added to the reaction mixture and refluxed the solution for 1 hour. The methanolic solution was concentrated and crude mass was purified by flash chromatography (Si-gel, 5% ethlyl acetate-hexane) to afford 640 mg (43.9%) of 6-Chloro-2-methyl-3,4-dihydro-2H-benzo[1,4]oxazine-2-carboxylic acid ethyl ester. ¹H-NMR (400 MHz, DMSO-d₆) δ (ppm): 6.71 (d, 1H), 6.55 (d, 1H), 6.50 (dd, 1H), 6.16 (bs, 1H), 4.06 (t, 2H), 3.57 (dd, 1H), 3.04 (d, 1H), 1.46 (s, 3H), 1.11 (t, 3H).

Step-5: 6-Chloro-2-methyl-3,4-dihydro-2H-benzo[1,4]oxazine-2-carboxylic acid

To a solution of 6-Chloro-2-methyl-3,4-dihydro-2H-benzo[1,4]oxazine-2-carboxylic acid ethyl ester (170 mg, 0.7 mmol) in THF: water (1:1) was added LiOH (70 mg, 1.7 mmol) under ice cold condition and stirred at room temperature for 2 hour. After completion of the reaction solvent was evaporated, added water, acidified with 1(N) HCl solution at 0° C. and extracted with ethyl acetate. The organic layer was washed with water, brine and dried over Na₂SO₄. Conecntration of organic layer afforded 100 mg (62.8%) of 6-Chloro-2-methyl-3,4-dihydro-2H-benzo[1,4]oxazine-2-carboxylic acid. LC/MS [M+H]⁺: 228.2

Step-6: 1-(6-Chloro-2-methyl-3,4-dihydro-2H-benzo[1,4]oxazine-2-carbonyl)-4-(4-fluoro-benzyl)-piperidine-4-carbonitrile

To a stirred solution of 6-Chloro-2-methyl-3,4-dihydro-2H-benzo[1,4]oxazine-2-carboxylic acid (100 mg, 0.44 mmol) was taken in dry DMF (1 ml) and was added EDCI (110 mg, 0.57 mmol), HOBT (30 mg, 0.22 mmol) and DIPEA (0.2 ml, 1.1 mmol) at 0° C. The reaction mixture was stirred at room temperature for 30 mins and 4-(4-fluoro-benzyl)-piperidine-4-carbonitrile (125 mg, 0.57 mmol) was added at 0° C. The reaction mixture was stirred at room temperature overnight and was added crushed ice, stirred, extracted with ethyl acetate. The organic layer was washed with water, brine and dried over Na₂SO₄. The organic layer was concentrated under reduced pressure and obtained crude mass was purified by flash chromatography (Si-gel, 20% ethyl acetate- hexane) to afford 80 mg (42.5%) of 1-(6-Chloro-2-methyl-3,4-dihydro-2H-benzo[1,4]oxazine-2-carbonyl)-4-(4-fluoro-benzyl)-piperidine-4-carbonitrile. LC/MS [M+H]⁺: 428.2. ¹H-NMR (400 MHz, DMSO-d₆) δ (ppm): 7.32 (m, 2H), 7.17 (t, 2H), 6.76 (d, 1H), 6.55 (d, 1H), 6.47 (d, 1H), 6.24 (bs, 1H), 4.50 (m, 5H), 3.67 (m, 1H), 2.94 (m, 4H), 1.79 (m, 2H), 1.46 (s, 3H). HPLC: 95.1%

### Example 141

### 1-(6-Chloro-7-dimethylamino-2-methyl-3,4-dihydro-2H-benzo[1,4]oxazine-2-carbonyl)-4-(4-fluoro-benzyl)-piperidine-4-carbonitrile

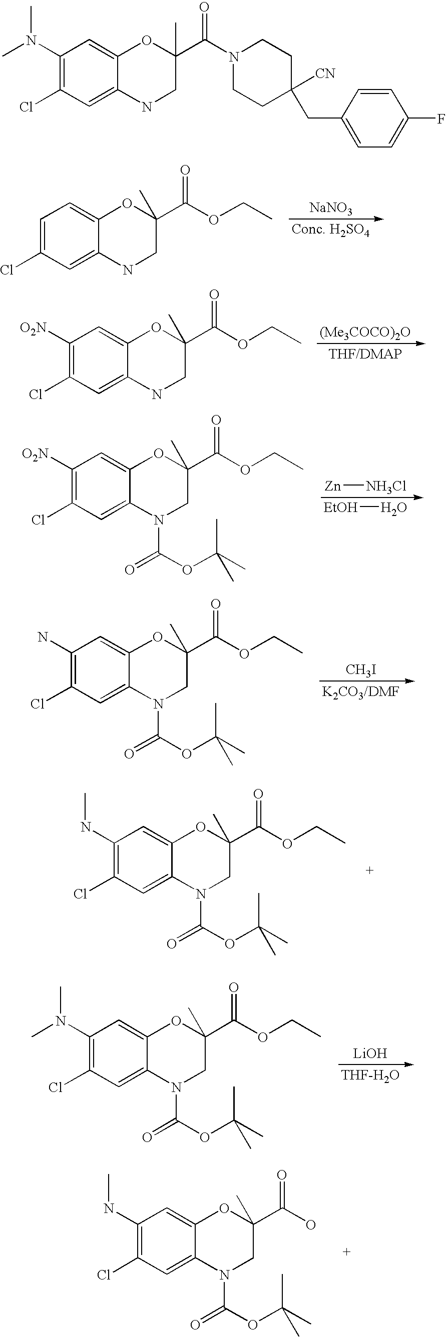

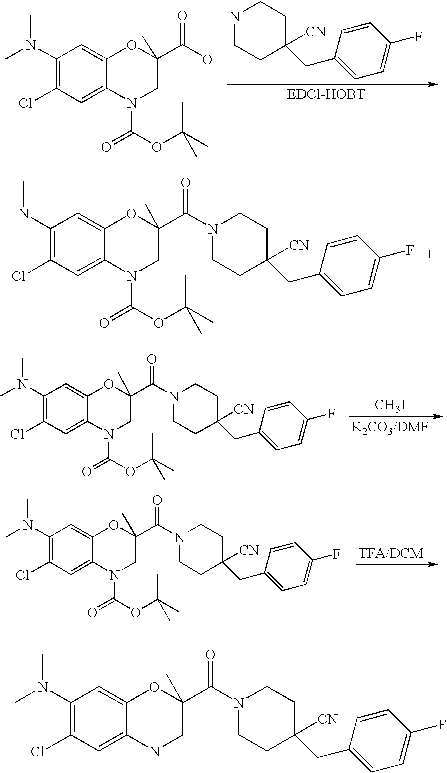

6-Chloro-2-methyl-3,4-dihydro-2H-benzo[1,4]oxazine-2-carboxylic acid ethyl ester was prepared by a process as described in example 140.
Step-1: 6-Chloro-2-methyl-7-nitro-3,4-dihydro-2H-benzo[1,4]oxazine-2-carboxylic acid ethyl ester

To a solution of 6-Chloro-2-methyl-3,4-dihydro-2H-benzo[1,4]oxazine-2-carboxylic acid ethyl ester (500 mg, 1.96 mmol) in concentrated H₂SO₄ (5 ml), NaNO₃ (166 mg, 1.96 mmol) was added portion wise at −15 to −20° C. Stirring was continued at this temperature for 1 hour. UV Intensity of the new spot generated was increased slightly after stirring of this reaction mass at −10° C. for half an hour and then no improvement was observed after stirring at −5° C. for another half an hour. This reaction mass was then poured into crushed ice and stirred well. The crude mass was extracted with ethyl acetate, washed with water, brine and dried over Na₂SO₄. The organic layer was concentrated and crude mass was purified by flash chromatography (Si-gel, 10% ethyl acetate-hexane) to get 240 mg (40.7%) of 6-Chloro-2-methyl-7-nitro-3,4-dihydro-2H-benzo[1,4]oxazine-2-carboxylic acid ethyl ester. ¹H-NMR (400 MHz, DMSO-d₆) δ (ppm): 7.55 (m, 2H), 6.73 (s, 1H), 4.09 (m, 2H), 3.75 (dd, 1H), 3.22 (m, 1H), 1.53 (s, 3H), 1.11 (t, 3H).

Step-2: 6-Chloro-2-methyl-7-nitro-2,3-dihydro-benzo[1,4]oxazine-2,4-dicarboxylic acid 4-tert-butyl ester 2-ethyl ester

To a solution of 6-Chloro-2-methyl-7-nitro-3,4-dihydro-2H-benzo[1,4]oxazine-2-carboxylic acid ethyl ester (240 mg, 0.8 mmol) in THF (10 ml), catalytic amount of DMAP and (BOC)₂O (0.2 ml, 0.8 mmol) was added at 0° C. The reaction mass was stirred under cold condition for 2.5 hour. The reaction mass was concentrated, added ethyl acetate. The organic part was washed with water, brine, dried over Na₂SO₄ and then concentrated to get 290 mg (90.4%) of 6-Chloro-2-methyl-7-nitro-2,3-dihydro-benzo[1,4]oxazine-2,4-dicarboxylic acid 4-tert-butyl ester 2-ethyl ester. LC/MS [M+H]⁺: 400.0.

Step-3: 7-Amino-6-chloro-2-methyl-2,3-dihydro-benzo[1,4]oxazine-2,4-dicarboxylic acid 4-tert-butyl ester 2-ethyl ester

To a solution of 6-Chloro-2-methyl-7-nitro-2,3-dihydro-benzo[1,4]oxazine-2,4-dicarboxylic acid 4-tert-butyl ester 2-ethyl ester (290 mg, 0.7 mmol) in THF-H₂O (4:1), Zn dust (331 mg, 5.1 mmol) and NH4Cl (116 mg, 2.2 mmol) was added. The reaction mixture was refluxed for 5 hour. The reaction mass was filtered over celite bed, added ethyl acetate, washed with water, brine and dried over Na₂SO₄. The organic layer was concentrated under reduced pressure to get 230 mg (88.6%) of 7-Amino-6-chloro-2-methyl-2,3-dihydro-benzo[1,4]oxazine-2,4-dicarboxylic acid 4-tert-butyl ester 2-ethyl ester. LC/MS [M+H]⁺: 371.4.

### Step-4

To a solution of 7-Amino-6-chloro-2-methyl-2,3-dihydro-benzo[1,4]oxazine-2,4-dicarboxylic acid 4-tert-butyl ester 2-ethyl ester (230 mg, 0.62 mmol) in dry THF added dry K₂CO₃ (214 mg, 1.6 mmol) and CH₃I (0.12 ml, 1.9 mmol) at 0° C. and then heated at 50° C. for 3 hour. The reaction mass was diluted with water, extracted with ethyl acetate, washed with water, brine and dried over Na₂SO₄. The organic layer was concentrated under reduced pressure and crude mass (210 mg) contains both the 6-Chloro-2-methyl-7-methylamino-2,3-dihydro-benzo[1,4]oxazine-2,4-dicarboxylic acid 4-tert-butyl ester 2-ethyl ester and 6-Chloro-7-dimethylamino-2-methyl-2,3-dihydro-benzo[1,4]oxazine-2,4-dicarboxylic acid 4-tert-butyl ester 2-ethyl ester . LC/MS [M+H]⁺: 399.2,385.4.

### Step-5

To a crude mixture of step 4 (210 mg, 0.53 mmol) in THF-H2O (3:1) was added LiOH (55 mg, 1.3 mmol) at 0° C. and stirred at room temperature for 2 hour. The reaction mixture was concentrated, diluted with water, extracted with ethyl acetate, washed with water, brine and dried over Na₂SO₄. The organic layer was concentrated under reduced pressure afforded 180 mg of a mixture of 6-Chloro-2-methyl-7-methylamino-2,3-dihydro-benzo[1,4]oxazine-2,4-dicarboxylic acid 4-tert-butyl ester and 6-Chloro-7-dimethylamino-2-methyl-2,3-dihydro-benzo[1,4]oxazine-2,4-dicarboxylic acid 4-tert-butyl ester. LC/MS [M+H]⁺: 371.2 and 357.2.

### Step-6

To a crude mixture of step-5 product (180 mg, 0.5 mmol) in dry DMF (2 ml) was added EDCI (125 mg, 0.65 mmol), HOBT (34 mg, 0.25 mmol) and DIPEA (0.25 ml, 1.5 mmol) at 0° C. The reaction mixture stirred at room temperature for 30 mins and was added 4-(4-Fluoro-benzyl)-piperidine-4-carbonitrile (106 mg, 0.5 mmol) at 0° C. The reaction mixture was stirred at room temperature overnight and added crushed ice, stirred, extracted with ethyl acetate, washed with water, brine and dried over Na₂SO₄. The organic layer was concentrated under reduced pressure and crude mass was purified by flash chromatography (10% ethyl acetate-hexane) to get 145 mg (50.8%) mixture of coupled product 6-Chloro-2-[4-cyano-4-(4-fluoro-benzyl)-piperidine-1-carbonyl]-2-methyl-7-methylamino-2,3-dihydro-benzo[1,4]oxazine-4-carboxylic acid tert-butyl ester and 6-Chloro-2-[4-cyano-4-(4-fluoro-benzyl)-piperidine-1-carbonyl]-7-dimethylamino-2-methyl-2,3-dihydro-benzo[1,4]oxazine-4-carboxylic acid tert-butyl ester. LC/MS [M+H]⁺: 571.2 and 557.4.

Step-7: 6-Chloro-2-[4-cyano-4-(4-fluoro-benzyl)-piperidine-1-carbonyl]-7-dimethylamino-2-methyl-2,3-dihydro-benzo[1,4]oxazine-4-carboxylic acid tert-butyl ester

To a solution of a mixture of product of step-6 (145 mg, 0.26 mmol) in THF was added dry K₂CO₃ (54 mg, 1.6 mmol) and CH₃I (0.01 ml, 0.13 mmol) at 0° C. and heated at 50° C. overnight. The reaction mass was diluted with water, extracted with ethyl acetate, washed with water, brine and dried over Na₂SO₄. The organic layer was concentrated under reduced pressure to afford 135 mg (90.9%) of 6-Chloro-2-[4-cyano-4-(4-fluoro-benzyl)-piperidine-1-carbonyl]-7-dimethylamino-2-methyl-2,3-dihydro-benzo[1,4]oxazine-4-carboxylic acid tert-butyl ester. LC/MS [M+H]⁺: 571.4.

Step-8: 1-(6-Chloro-7-dimethylamino-2-methyl-3,4-dihydro-2H-benzo[1,4]oxazine-2-carbonyl)-4-(4-fluoro-benzyl)-piperidine-4-carbonitrile

To a stirred solution of 6-Chloro-2-[4-cyano-4-(4-fluoro-benzyl)-piperidine-1-carbonyl]-7-dimethylamino-2-methyl-2,3-dihydro-benzo[1,4]oxazine-4-carboxylic acid tert-butyl ester (135 mg, 0.23 mmol) in DCM (10 ml) was added TFA (0.18 ml, 2.4 mmol) at 0° C. and stirred at room temperature for 2 h. The reaction mixture was completely evaporated under reduced pressure, added small amount of water, basified with 1(N) NaOH solution, extracted with ethyl acetate, washed with water, brine and dried over Na₂SO₄. The organic layer was concentrated and crude mass was washed with hexane to afford 20 mg (18.5%) of J 1-(6-Chloro-7-dimethylamino-2-methyl-3,4-dihydro-2H-benzo[1,4]oxazine-2-carbonyl)-4-(4-fluoro-benzyl)-piperidine-4-carbonitrile. LC/MS [M+H]⁺: 471.4. ¹H-NMR (400 MHz, DMSO-d₆) δ (ppm): 7.32 (bs, 2H), 7.17 (m, 2H), 6.66 (s, 1H), 6.59 (s, 1H), 5.8 1(bs, 1H), 4.60 (m, 4H), 3.62 (d, 1H), 2.90 (m, 3H), 2.56 (s, 6H), 1.81 (m, 2H), 1.46 (s,3H). HPLC: 93.5%.

### Example 142

### 6-Chloro-2-[4-cyano-4-(4-fluoro-benzyl)-piperidine-1-carbonyl]-7-dimethylamino-2,3-dihydro-benzo[1,4]oxazine-4-carbonitrile

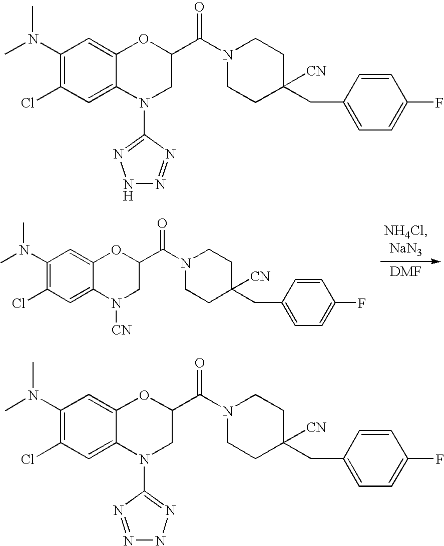

6-Chloro-2-[4-cyano-4-(4-fluoro-benzyl)-piperidine-1-carbonyl]-7-dimethylamino-2,3-dihydro-benzo[1,4]oxazine-4-carbonitrile was prepared by a process as described in example 76.

Step-1: 1-[6-Chloro-7-dimethylamino-4-(2H-tetrazol-5-yl)-3,4-dihydro-2H-benzo[1,4]oxazine-2-carbonyl]-4-(4-fluoro-benzyl)-piperidine-4-carbonitrile

To the stirred solution of 6-Chloro-2-[4-cyano-4-(4-fluoro-benzyl)-piperidine-1-carbonyl]-7-dimethylamino-2,3-dihydro-benzo[1,4]oxazine-4-carbonitrile (300 mg, 0.62 mmol) in DMF were added NH₄Cl (134 mg, 2.5 mmol) and NaN₃ (162 mg, 2.5 mmol). The reaction mixture was heated overnight at 120° C. The reaction mixture was diluted with ethyl acetate, washed with water, brine and dried over Na₂SO₄. The organic layer was concentrated under reduced pressure and crude mass was purified by flash chromatography (Si-gel, 2.0% MeOH-DCM) to afford 25 mg (7.7%) of 1-[6-Chloro-7-dimethylamino-4-(2H-tetrazol-5-yl)-3,4-dihydro-2H-benzo[1,4]oxazine-2-carbonyl]-4-(4-fluoro-benzyl)-piperidine-4-carbonitrile. LC/MS [M+H]⁺: 525.4. ¹H-NMR (400 MHz, DMSO-d₆) δ (ppm): 8.0-6.7 (m, 7H), 5.35-2.93 (m, 10H), 2.67 (s, 6H), 1.98-1.52 (m, 3H). LCMS purity: 90.3%.

### Example 143

### 1-[6-Chloro-7-(2-methoxy-phenyl)-3,4-dihydro-2H-benzo[1,4]oxazine-2-carbonyl]-4-(4-fluoro-benzyl)-piperidine-4-carbonitrile

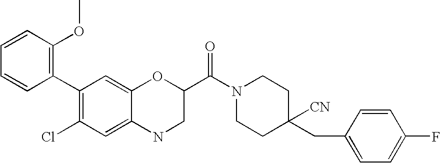

1-[6-Chloro-7-(2-methoxy-phenyl)-3,4-dihydro-2H-benzo[1,4]oxazine-2-carbonyl]-4-(4-fluoro-benzyl)-piperidine-4-carbonitrile was prepared by a process similar to that described in example 128. LC/MS [M+H]⁺: 509.4. ¹H-NMR (400 MHz, DMSO-d₆) δ (ppm): 7.32 (m, 3H), 7.17 (m, 2H), 7.06 (m, 2H), 6.96 (m, 1H), 6.67 (bs, 1H), 6.60 (bs, 1H), 6.18(bs, 1H), 4.96 (bs, 1H), 4.39 (m, 1H), 4.11 (m, 1H), 3.70 (s, 3H), 3.43 (m, 1H), 3.14 (m, 1H), 2.92 (m, 2H), 2.69 (m, 1H), 1.98 (s, 1H), 1.73 (m, 3H), 1.54 (m, 1H).

HPLC: 90.3%.

### Example 144

### 1-(6-Chloro-7-pyridin-2-yl-3,4-dihydro-2H-benzo[1,4]oxazine-2-carbonyl)-4-(4-fluoro-benzyl)-piperidine-4-carbonitrile

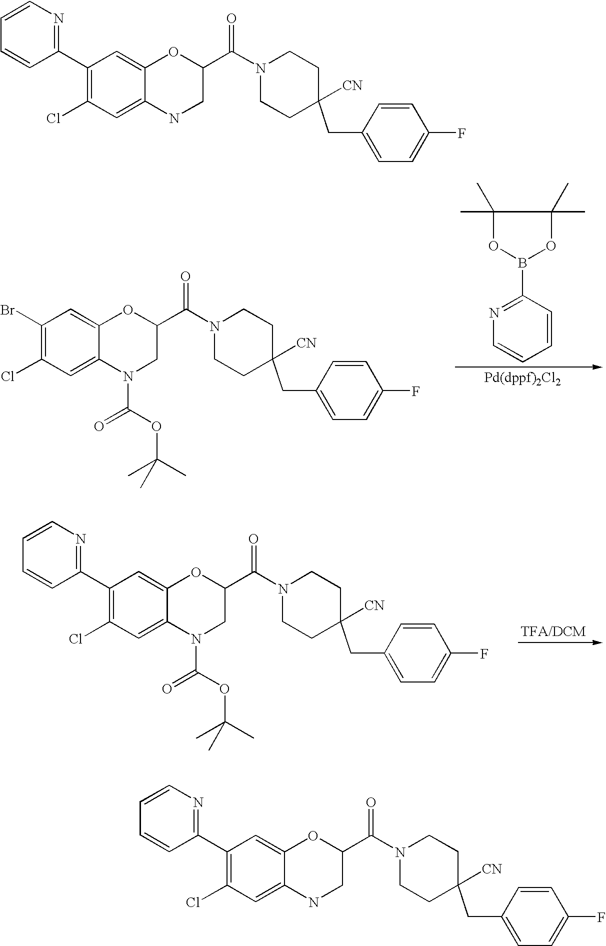

Step 1: 6-Chloro-2-[4-cyano-4-(4-fluoro-benzyl)-piperidine-1-carbonyl]-7-pyridin-2-yl-2,3-dihydro-benzo[1,4]oxazine-4-carboxylic acid tert-butyl ester

A solution of 7-Bromo-6-chloro-2-[4-cyano-4-(4-fluoro-benzyl)-piperidine-1-carbonyl]-2,3-dihydro-benzo[1,4]oxazine-4-carboxylic acid tert-butyl ester (250 mg, 0.42 mmol), 2-[4,4,5,5-Tetramethyl-[1,3,2]dioxaborolan-2-yl)-pyridine (112 mg, 0.54 mmol), Na₂CO₃ (90 mg, 0.84 mmol) in toluene-water was degassed for 1 hour and was added [Pd (dppf)₂Cl₂] was added at argon atmosphere. The reaction mixture was heated at 90° C. for overnight and diluted with water. The organics was extracted with ethyl acetate. The organic layer was washed with water, brine and dried over Na₂SO₄. The volatile components were removed under reduced pressure and purified by column chromatography (Si-gel, 30% ethyl acetate- hexane) to afford 35 mg (14%) of 6-Chloro-2-[4-cyano-4-(4-fluoro-benzyl)-piperidine-1-carbonyl]-7-pyridin-2-yl-2,3-dihydro-benzo[1,4]oxazine-4-carboxylic acid tert-butyl ester. LC/MS [M+H]⁺: 591.2.

Step 2: 1-(6-Chloro-7-pyridin-2-yl-3,4-dihydro-2H-benzo[1,4]oxazine-2-carbonyl)-4-(4-fluoro-benzyl)-piperidine-4-carbonitrile

To a solution of 6-Chloro-2-[4-cyano-4-(4-fluoro-benzyl)-piperidine-1-carbonyl]-7-pyridin-2-yl-2,3-dihydro-benzo[1,4]oxazine-4-carboxylic acid tert-butyl ester (35 mg, 0.06 mmol) in DCM was added TFA (0.5 ml) and stirred at RT for 1 hours. The volatile components were removed under reduced pressure and residue was diluted with water. It was basified and extracted with ethyl acetate. The organic layer was washed with water, brine and dried over Na₂SO₄. The organic layer was concentrated and washed with hexane to afford 15 mg (51.6%) of 1-[6-Chloro-7-(1-methyl-1H-pyrazol-4-yl)-3,4-dihydro-2H-benzo[1,4]oxazine-2-carbonyl]-4-(4-fluoro-benzyl)-piperidine-4-carbonitrile. LC/MS [M+H]⁺: 491.0. ¹H-NMR (400 MHz, DMSO-d₆) δ (ppm): 8.61 (bs, 1H), 7.81 (m, 1H), 7.62 (m, 1H), 7.33 (bs, 3H), 7.18 (bs, 2H), 6.99 (s, 1H), 6.71 (s, 1H), 6.44 (s, 1H), 5.00 (m, 1H), 4.60 (m, 1H), 4.12 (m, 1H), 3.42(m, 1H), 2.95 (s, 2H), 2.68(m, 1H), 1.79 (m, 3H), 1.54 (m, 1H), 1.23 (m, 1H), 0.85 (m, 1H). HPLC: 85.4%.

### Example 145

### 1-(7-Acetyl-6-chloro-3,4-dihydro-2H-benzo[1,4]oxazine-2-carbonyl)-4-(4-fluoro-benzyl)-piperidine-4-carbonitrile

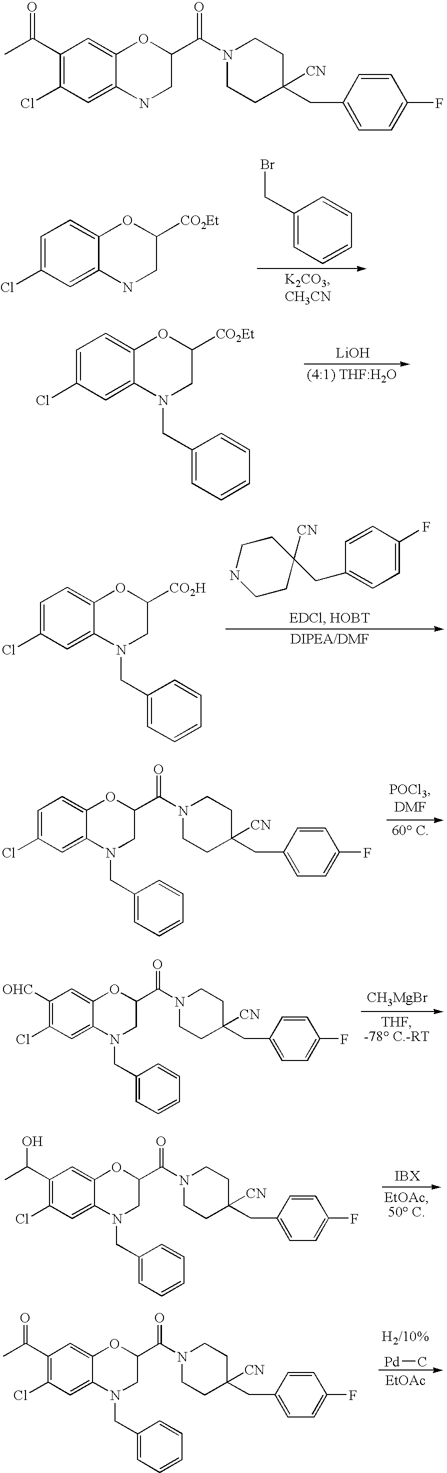

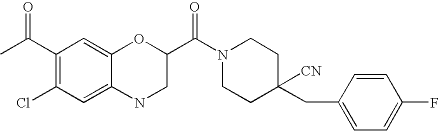

Step-1: 4-Benzyl-6-chloro-3,4-dihydro-2H-benzo[1,4]oxazine-2-carboxylic acid ethyl ester

To a solution of 6-Chloro-3,4-dihydro-2H-benzo[1,4]oxazine-2-carboxylic acid ethyl ester (1.8 g, 7.45 mmol) in acetonitrile were added K₂CO₃ (2.2 g, 15.64 mmol) and Benzyl bromide (1.0 ml, 8.19 mmol). The reaction mixture was refluxed overnight, concentrated and extracted with ethyl acetate. The organic layer was washed with water, brine and dried over Na₂SO₄. It was concentrated and purified by column chromatography (Silica gel, 4% ethyl acetate-hexane) to afford 1.48 g (59.9%) of 4-Benzyl-6-chloro-3,4-dihydro-2H-benzo[1,4]oxazine-2-carboxylic acid ethyl ester. ¹H-NMR (400 MHz, DMSO-d₆) δ (ppm): 7.34 (m, 2H), 7.25 (m, 3H), 6.80 (m, 1H), 6.66 (m, 1H), 6.60 (m, 1H), 5.09 (bs, 1H), 4.52 (m, 1H), 4.38 (m, 1H), 4.09 (m, 2H), 3.55 (m, 2H), 1.13(m, 3H).

Step-2: 4-Benzyl-6-chloro-3,4-dihydro-2H-benzo[1,4]oxazine-2-carboxylic acid

To a solution of 4-Benzyl-6-chloro-3,4-dihydro-2H-benzo[1,4]oxazine-2-carboxylic acid ethyl ester (1 g, 3.0 mmol) in (4:1) THF:H₂O was added LiOH (252 mg, 6.0 mmol) at cold condition. The reaction mixture was stirred at RT for 2 hours. It was concentrated, diluted with water and acidified with aq. HCl in cold condition. It was extracted with ethyl acetate and organic layer was washed with water, brine and dried over Na₂SO₄. The organic layer was concentrated to afford 950 mg (crude) of 4-Benzyl-6-chloro-3,4-dihydro-2H-benzo[1,4]oxazine-2-carboxylic acid. ¹H-NMR (400 MHz, DMSO-d₆) δ (ppm): 13.17 (hump, 1H), 7.33 (m, 2H), 7.28 (m, 3H), 6.78 (m, 1H), 6.58 (m, 2H), 4.96 (s, 1H), 4.47 (m, 2H), 3.53 (m, 2H).

Step-3: 1-(4-Benzyl-6-chloro-3,4-dihydro-2H-benzo[1,4]oxazine-2-carbonyl)-4-(4-fluoro-benzyl)-piperidine-4-carbonitrile

To a solution of 4-Benzyl-6-chloro-3,4-dihydro-2H-benzo[1,4]oxazine-2-carboxylic acid (950 mg, 3.13 mmol) in DMF was added DIPEA (1.7 ml, 9.38 mmol) drop wise at cold condition. EDCI (900 mg, 4.69 mmol), HOBT (506 mg, 3.75 mmol) were then added to the reaction mixture and stirred for 0.5 hour. 4-(4-Fluoro-benzyl)-piperidine-4-carbonitrile (683 mg, 3.13 mmol) was added to the reaction mixture and stirred overnight at RT. It was diluted with water and extracted with ethyl acetate. Organic layer was washed with water, brine and dried over Na₂SO₄. It was concentrated and purified by column chromatography (Silica gel, 20% ethyl acetate-hexane) to afford 1.2 g (76%) of 1-(4-Benzyl-6-chloro-3,4-dihydro-2H-benzo[1,4]oxazine-2-carbonyl)-4-(4-fluoro-benzyl)-piperidine-4-carbonitrile. LC/MS [M+H]⁺: 504.2.

Step-4: 1-(4-Benzyl-6-chloro-7-formyl-3,4-dihydro-2H-benzo[1,4]oxazine-2-carbonyl)-4-(4-fluoro-benzyl)-piperidine-4-carbonitrile

POCl₃ (0.5 ml, 5.34 mmol) was added drop wise to DMF (3 ml) at ice-cold condition and stirred for 0.5 hour. 1-(4-Benzyl-6-chloro-3,4-dihydro-2H-benzo[1,4]oxazine-2-carbonyl)-4-(4-fluoro-benzyl)-piperidine-4-carbonitrile (900 mg, 1.78 mmol) was then added to the DMF-POCl₃ and heated at 60° C. overnight. The reaction mixture was quenched with ice-water and extracted with ethyl acetate. The organic layer was dried over Na₂SO₄ and concentrated under reduced pressure to afford 770 mg (81.3%) 1-(4-Benzyl-6-chloro-7-formyl-3,4-dihydro-2H-benzo[1,4]oxazine-2-carbonyl)-4-(4-fluoro-benzyl)-piperidine-4-carbonitrile. ¹H-NMR (400 MHz, DMSO-d₆) δ (ppm): 10.0 (m, 1H), 7.29 (m, 7H), 7.19 (m, 3H), 6.78 (s, 1H), 5.25 (bs, 1H), 4.75 (m, 1H), 4.64 (m, 1H), 4.35 (m, 1H), 4.05(m, 1H), 3.61 (s, 2H), 2.95 (m, 3H), 2.70 (m, 1H), 1.80 (m, 3H), 1.53 (m, 1H).

Step-5: 1-[4-Benzyl-6-chloro-7-(1-hydroxy-ethyl)-3,4-dihydro-2H-benzo[1,4]oxazine-2-carbonyl]-4-(4-fluoro-benzyl)-piperidine-4-carbonitrile

To a solution of 1-(4-Benzyl-6-chloro-7-formyl-3,4-dihydro-2H-benzo[1,4]oxazine-2-carbonyl)-4-(4-fluoro-benzyl)-piperidine-4-carbonitrile (200 mg, 0.38 mmol) in THF was added [3(M) MeMgBr] (0.15 ml, 0.41 mmol) drop wise at −78° C. The reaction mixture was stirred overnight at RT, quenched with saturated aq. NH₄Cl solution and extracted with ethyl acetate. The organic layer was washed with water, brine and dried over Na₂SO₄. The organic solution was concentrated and purified by column chromatography (Silica gel, 30% ethyl acetate-hexane) to afford 138 mg (68%) of 1-[4-Benzyl-6-chloro-7-(1-hydroxy-ethyl)-3,4-dihydro-2H-benzo[1,4]oxazine-2-carbonyl]-4-(4-fluoro-benzyl)-piperidine-4-carbonitrile. LC/MS [M+H]⁺: 548.6.

Step-6: 1-(7-Acetyl-4-benzyl-6-chloro-3,4-dihydro-2H-benzo[1,4]oxazine-2-carbonyl)-4-(4-fluoro-benzyl)-piperidine-4-carbonitrile

To a solution of 1-[4-Benzyl-6-chloro-7-(1-hydroxy-ethyl)-3,4-dihydro-2H-benzo[1,4]oxazine-2-carbonyl]-4-(4-fluoro-benzyl)-piperidine-4-carbonitrile (300 mg, 0.55 mmol) in ethyl acetate was added IBX (307 mg, 1.09 mmol) at cold condition and heated at 50° C. overnight. The precipitated solid was filtered off and mother liquor was concentrated. The crude organics was purified by column chromatography (Silica gel, 30% ethyl acetate-hexane) to afford 130 mg (43.3%) of 1-(7-Acetyl-4-benzyl-6-chloro-3,4-dihydro-2H-benzo[1,4]oxazine-2-carbonyl)-4-(4-fluoro-benzyl)-piperidine-4-carbonitrile. LC/MS [M+H]⁺: 546.6.

Step-7: 1-(7-Acetyl-6-chloro-3,4-dihydro-2H-benzo[1,4]oxazine-2-carbonyl)-4-(4-fluoro-benzyl)-piperidine-4-carbonitrile

To a solution of 1-(7-Acetyl-4-benzyl-6-chloro-3,4-dihydro-2H-benzo[1,4]oxazine-2-carbonyl)-4-(4-fluoro-benzyl)-piperidine-4-carbonitrile (130 mg, 0.24 mmol) in HPLC ethyl acetate was added 10% Pd—C. The reaction mixture was hydrogenated at 50 psi for 5 hours. The catalyst was filtered off and mother liquor was concentrated. The crude organics was purified by column chromatography (Silica gel, 40% ethyl acetate-hexane) to afford 15 mg (13.7%) of 1-(7-Acetyl-6-chloro-3,4-dihydro-2H-benzo[1,4]oxazine-2-carbonyl)-4-(4-fluoro-benzyl)-piperidine-4-carbonitrile. LC/MS [M+H]⁺: 456.2. ¹H-NMR (400 MHz, DMSO-d₆) δ (ppm): 7.61-6.62 (m, 7H), 5.00 (m, 1H), 4.37 (m, 1H), 4.10 (m, 1H), 2.96 (m, 3H), 2.66 (m, 1H), 1.90 (m, 1H), 1.72 (m, 4H), 1.53 (m, 2H), 1.24 (m, 2H). HPLC: 91.9%

### Example 146

### 1-(6-Chloro-7-oxazol-5-yl-3,4-dihydro-2H-benzo[1,4]oxazine-2-carbonyl)-4-(4-fluoro-benzyl)-piperidine-4-carbonitrile

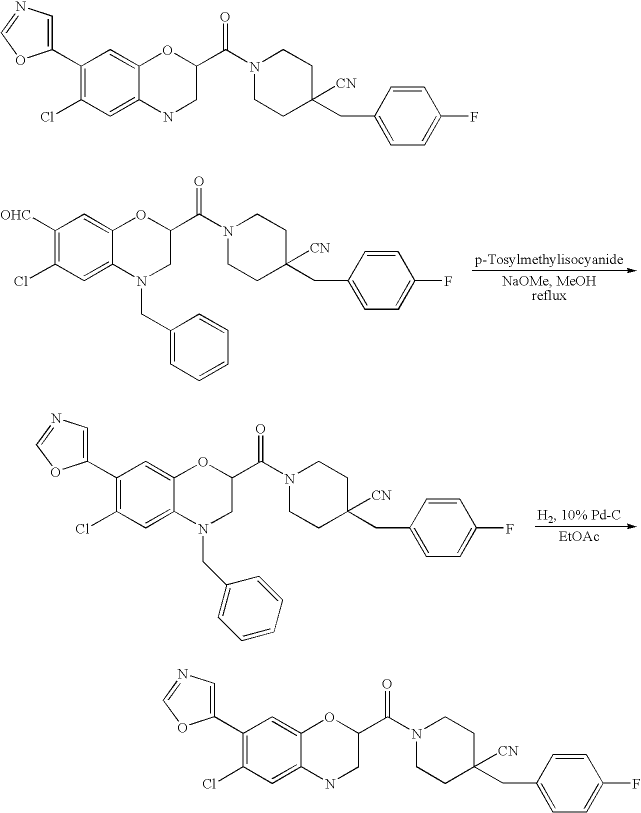

4-Benzyl-6-chloro-3,4-dihydro-2H-benzo[1,4]oxazine-2-carboxylic acid ethyl ester was prepared by a process as described in example 144.
Step-1: 1-(4-Benzyl-6-chloro-7-oxazol-5-yl-3,4-dihydro-2H-benzo[1,4]oxazine-2-carbonyl)-4-(4-fluoro-benzyl)-piperidine-4-carbonitrile

To a mixture of 1-(6-Chloro-7-formyl-3,4-dihydro-2H-benzo[1,4]oxazine-2-carbonyl)-4-(4-fluoro-benzyl)-piperidine-4-carbonitrile (1 g, 1.87 mmol) and sodium methoxe (305 mg, 5.6 mmol) in methanol was added p-Tosylmethylisocyanide (441 mg, 2.26 mmol). The reaction mixture was refluxed one hour. The reaction mixture was concentrated, diluted with water and extracted with ethyl acetate. The organic layer was concentrated to afford 461 mg (43%) of 1-(4-Benzyl-6-chloro-7-oxazol-5-yl-3,4-dihydro-2H-benzo[1,4]oxazine-2-carbonyl)-4-(4-fluoro-benzyl)-piperidine-4-carbonitrile. LC/MS [M+H]⁺: 571.0.

Step-2: 1-(6-Chloro-7-oxazol-5-yl-3,4-dihydro-2H-benzo[1,4]oxazine-2-carbonyl)-4-(4-fluoro-benzyl)-piperidine-4-carbonitrile

To a solution of 1-(4-Benzyl-6-chloro-7-oxazol-5-yl-3,4-dihydro-2H-benzo[1,4]oxazine-2-carbonyl)-4-(4-fluoro-benzyl)-piperidine-4-carbonitrile (200 mg, 0.35 mmol) in HPLC ethyl acetate was added 10% Pd—C. The reaction mixture was hydrogenated at 50 psi for 5 hours. The catalyst was filtered off and mother liquor was concentrated. The crude organics was purified by column chromatography (Silica gel, 45% ethyl acetate-hexane) to afford 17 mg (10%) of 1-(6-Chloro-7-oxazol-5-yl-3,4-dihydro-2H-benzo[1,4]oxazine-2-carbonyl)-4-(4-fluoro-benzyl)-piperidine-4-carbonitrile. LC/MS [M+H]⁺: 481.6. ¹H-NMR (400 MHz, DMSO-d₆) δ (ppm): 8.41 (m, 1H), 7.49 (bs, 1H), 7.34 (bs, 2H), 7.19 (t, 2H) 7.08 (m, 1H), 6.74 (s 1H), 6.59 (bs 1H), 5.03 (bs 1H), 4.38 (m, 1H), 4.11 (m, 1H), 3.43-2.67 (m, 6H), 1.89-1.54 (m, 4H). HPLC: 88.5%.

### Example 147

### 1-(4-Chloro-2-methyl-3,6,7,8-tetrahydro-9-oxa-1,3,6-triaza-cyclopenta[a]naphthalene-8-carbonyl)-4-(4-fluoro-benzyl)-piperidine-4-carbonitrile

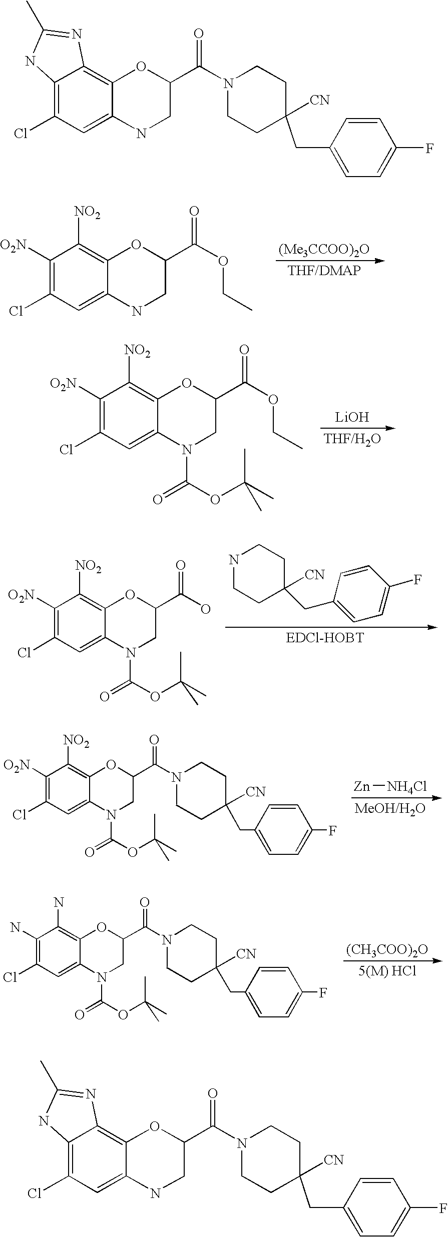

6-Chloro-7,8-dinitro-3,4-dihydro-2H-benzo[1,4]oxazine-2-carboxylic acid ethyl ester was prepared by a process similar to that described above in example 51.
Step-1: 6-Chloro-7,8-dinitro-2,3-dihydro-benzo[1,4]oxazine-2,4-dicarboxylic acid 4-tert-butyl ester 2-ethyl ester

To a solution of 6-Chloro-7,8-dinitro-3,4-dihydro-2H-benzo[1,4]oxazine-2-carboxylic acid ethyl ester (3 g, 9.05 mmol) in THF (40 ml) was added DMAP (111 mg, 0.91 mmol) and di-tert-butyl dicarbonate (4.6 ml, 21.07 mmol) drop wise at room temperature. The reaction mixture was stirred at room temperature for 5 h and concentrated under reduced pressure. The crude mass was purified by column chromatography (Si-gel, 5% ethyl acetate-hexane) to afford 1.29 g (33%) of 6-Chloro-7,8-dinitro-2,3-dihydro-benzo[1,4]oxazine-2,4-dicarboxylic acid 4-tert-butyl ester 2-ethyl ester. LC/MS [M+NH₃]⁺: 449.2.

Step-2: 6-Chloro-7,8-dinitro-2,3-dihydro-benzo[1,4]oxazine-2,4-dicarboxylic acid 4-tert-butyl ester

To a stirred suspension of 6-Chloro-7,8-dinitro-2,3-dihydro-benzo[1,4]oxazine-2,4-dicarboxylic acid 4-tert-butyl ester 2-ethyl ester (1.29 g, 2.99 mmol) in THF-H₂O (3:1), LiOH (313 mg, 7.47 mmol) was added portion wise under cold condition and stirred at room temperature for 2 hour. The reaction mixture was concentrated under reduced pressure, diluted with water. The solution was acidified with 50% acetic acid solution under cold condition. The organic mass was extracted with ethyl acetate, washed with water and brine. The organic layer was dried over Na₂SO₄ and concentrated under reduced pressure. The crude mass was then tripped with toluene thrice to get 1.16 g (96%) of 6-Chloro-7,8-dinitro-2,3-dihydro-benzo[1,4]oxazine-2,4-dicarboxylic acid 4-tert-butyl ester. ¹H-NMR (400 MHz, DMSO-d₆) δ (ppm): 8.15 (s, 1H), 5.31 (s, 1H), 4.54 (d,1H), 3.66 (d,1H), 1.38 (s, 9H).

Step-3: 6-Chloro-2-[4-cyano-4-(4-fluoro-benzyl)-piperidine-1-carbonyl]-7,8-dinitro-2,3-dihydro-benzo[1,4]oxazine-4-carboxylic acid tert-butyl ester

To a stirred solution of 6-Chloro-7,8-dinitro-2,3-dihydro-benzo[1,4]oxazine-2,4-dicarboxylic acid 4-tert-butyl ester (1.16 g, 2.87 mmol) in DMF (5.0 ml) was added EDCI (715 mg, 3.73 mmol), HOBT (194 mg, 1.44 mmol) & DIPEA (1.5 ml, 8.61 mmol) at 0° C. and stirred at room temperature for 30 minutes. 4-(4-Fluoro-benzyl)-piperidine-4-carbonitrile (627 mg, 2.87 mmol) was added at 0° C. to the previous solution and stirred at room temperature overnight. The reaction mixture was quenched with crushed ice, stirred, extracted with ethyl acetate. The organic layer was washed with water and brine solution. The organic layer was dried over Na₂SO₄ and concentrated under reduced pressure. The crude mass was purified by column chromatography (Si-gel, 25% ethyl acetate-hexane) to afford 900 mg (51.9%) of 6-Chloro-2-[4-cyano-4-(4-fluoro-benzyl)-piperidine-1-carbonyl]-7,8-dinitro-2,3-dihydro-benzo[1,4]oxazine-4-carboxylic acid tert-butyl ester. LC/MS [M+NH₃]⁺: 621.6.

Step-4: 7, 8-Diamino-6-chloro-2-[4-cyano-4-(4-fluoro-benzyl)-piperidine-1-carbonyl]-2,3-dihydro-benzo[1,4]oxazine-4-carboxylic acid tent-butyl ester

To a stirred suspension of 6-Chloro-2-[4-cyano-4-(4-fluoro-benzyl)-piperidine-1-carbonyl]-7,8-dinitro-2,3-dihydro-benzo[1,4]oxazine-4-carboxylic acid tert-butyl ester G (900 mg, 1.49 mmol) in 25 ml MeOH—H₂O (4:1), Zn-dust (1.95 g, 29.8 mmol) and NH₄Cl (797 mg, 14.9 mmol) was added at 0° C. The reaction mass was stirred at room temperature overnight. The reaction mass was filtered over a celite bed and concentrated. To that added water, extracted with ethyl acetate, washed with water, brine, dried with Na₂SO₄ and concentrated to get 600 mg (74%) of 7,8-Diamino-6-chloro-2-[4-cyano-4-(4-fluoro-benzyl)-piperidine-1-carbonyl]-2,3-dihydro-benzo[1,4]oxazine-4-carboxylic acid tert-butyl ester. LC/MS [M+H]⁺: 544.0.

Step-5: 1-(4-Chloro-2-methyl-3,6,7,8-tetrahydro-9-oxa-1,3,6-triaza-cyclopenta[a]naphthalene-8-carbonyl)-4-(4-fluoro-benzyl)-piperidine-4-carbonitrile

To a stirred solution of 7,8-Diamino-6-chloro-2-[4-cyano-4-(4-fluoro-benzyl)-piperidine-1-carbonyl]-2,3-dihydro-benzo[1,4]oxazine-4-carboxylic acid tert-butyl ester (100 mg, 0.18 mmol) in acetic anhydride (1.5 ml) was added 5(M) HCl (0.5 ml) and heated at 80° C. for 2 hours. The reaction mixture was concentrated, basified with saturated NaHCO₃ solution and extracted with ethyl acetate. The organic layer was washed with water, brine, dried with Na₂SO₄ and concentrated under reduced pressure. The crude mass was purified by column chromatography (Si-gel, 1.8% MeOH-DCM) to get 25 mg (29.7%) of 1-(4-Chloro-2-methyl-3,6,7,8-tetrahydro-9-oxa-1,3,6-triaza-cyclopenta[a]naphthalene-8-carbonyl)-4-(4-fluoro-benzyl)-piperidine-4-carbonitrile. LC/MS [M+H]⁺: 468.6. ¹H-NMR (400 MHz, DMSO-d₆) δ (ppm): 7.35 (m, 2H), 7.20 (m, 2H), 6.31 (m, 1H), 5.40 (m, 1H), 4.92 (m, 2H), 4.42 (m, 2H), 4.25 (m, 2H), 3.20 (m, 1H), 2.97 (d, 2H), 2.76 (m, 1H), 2.45 (s, 3H), 1.80 (m, 2H), 1.53 (m, 1H). HPLC: 94.36%.

### Example 148

### 1-(4-Chloro-3,6,7,8-tetrahydro-9-oxa-1,3,6triazacyclopenta[a]naphthalene-8-carbonyl)-4-(4-fluoro-benzyl)-piperidine-4-carbonitrile

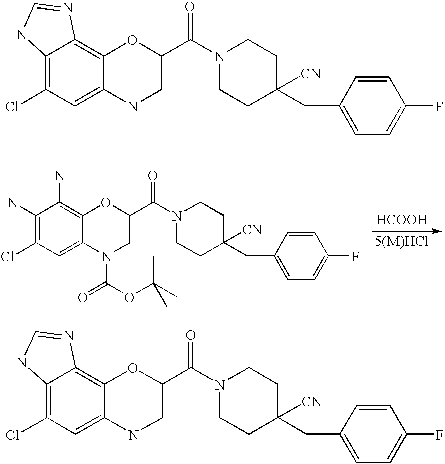

7,8-Diamino-6-chloro-2-[4-cyano-4-(4-fluoro-benzyl)-piperidine-1-carbonyl]-2,3-dihydro-benzo[1,4]oxazine-4-carboxylic acid tert-butyl ester was prepared by a process as described in example 145.
Step-1: 1-(4-Chloro-3,6,7,8-tetrahydro-9-oxa-1,3,6triazacyclopenta[a]naphthalene-8-carbonyl)-4-(4-fluoro-benzyl)-piperidine-4-carbonitrile

To a stirred solution of 7,8-Diamino-6-chloro-2-[4-cyano-4-(4-fluoro-benzyl)-piperidine-1-carbonyl]-2,3-dihydro-benzo[1,4]oxazine-4-carboxylic acid tert-butyl ester (95 mg, 0.17 mmol) in formic acid was added 5(M) HCl (0.5 ml) and heated at 80° C. for 2 hours. The reaction mixture was concentrated, basified with saturated NaHCO₃ solution and extracted with ethyl acetate. The organic layer was washed with water, brine, dried over Na₂SO₄ and concentrated under reduced pressure. The crude mass was purified by column chromatography (Si-gel, 2% MeOH-DCM) to get 15 mg (19.4%) of 1-(4-Chloro-3,6,7,8-tetrahydro-9-oxa-1,3,6-triazacyclopenta[a]naphthalene-8-carbonyl)-4-(4-fluoro-benzyl)-piperidine-4-carbonitrile. LC/MS [M+H]⁺: 454.2. ¹H-NMR (400 MHz, DMSO-d₆) δ (ppm): 8.04 (s, 1H), 7.35 (m, 2H), 7.21 (m, 2H), 6.38 (m, 1H), 5.40 (m, 1H), 5.03 (m, 2H), 4.54 (m, 1H), 4.42 (m, 2H), 4.20 (m, 1H), 3.19 (m, 2H), 2.95 (m, 2H), 2.76 (m, 1H), 1.80 (m, 2H). HPLC: 93.9%.

### Example 149

### 6-Chloro-2-[4-cyano-4-(4-fluoro-benzyl)-piperidine-1-carbonyl]-3,4-dihydro-2H-benzo[1,4]oxazine-7-carboxylic acid methylamide

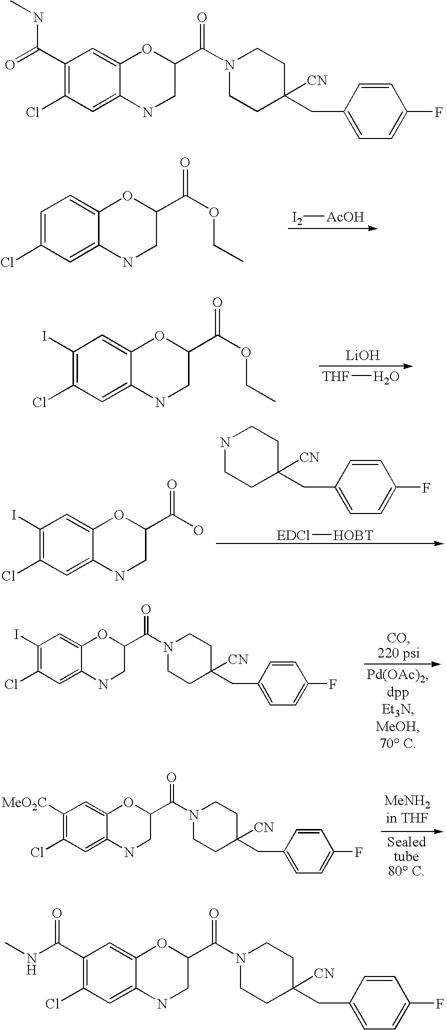

Step-1: 6-Chloro-7-iodo-3,4-dihydro-2H-benzo[1,4]oxazine-2-carboxylic acid ethyl ester

To a stirred solution of 6-Chloro-3,4-dihydro-2H-benzo[1,4]oxazine-2-carboxylic acid ethyl ester (5 g, 20.68 mmol) in acetic acid was added iodine (5.26 g, 20.68 mmol) drop wise at 10° C. The reaction mixture was stirred for 30 min at same temperature. The reaction mixture was poured in ice-water, extracted the compound with ethyl acetate, washed with water, brine, dried over Na₂SO4 and concentrated under reduced pressure. Thus obtained crude was purified through repeated column chromatography (Si-gel, 20% DCM-Hex) to afford 1.1 g (14.5%) of 6-Chloro-7-iodo-3,4-dihydro-2H-benzo[1,4]oxazine-2-carboxylic acid ethyl ester . LC/MS [M+H]⁺: 368.2.

Step-2: 6-Chloro-7-iodo-3,4-dihydro-2H-benzo[1,4]oxazine-2-carboxylic acid

To a stirred solution of 6-Chloro-7-iodo-3,4-dihydro-2H-benzo[1,4]oxazine-2-carboxylic acid ethyl ester (400 mg, 1.09 mmol) in THF: Water (3:1) was added LiOH (46 mg, 1.09 mmol) and stirred the reaction mixture for 2 h at room temperature. The reaction mixture was concentrated, diluted with water, acidified with dilute acetic acid and extracted the organics with ethyl acetate. The organic layer was washed with water, brine, dried over Na₂SO4 and concentrated under reduced pressure. Washed the crude with hexane to afford 260 mg (70.3%) of 6-Chloro-7-iodo-3,4-dihydro-2H-benzo[1,4]oxazine-2-carboxylic acid. The material was used in forward reaction without any characterization.

Step-3: 1-(6-Chloro-7-iodo-3,4-dihydro-2H-benzo[1,4]oxazine-2-carbonyl)-4-(4-fluoro-benzyl)-piperidine-4-carbonitrile

To a solution of 6-Chloro-7-iodo-3,4-dihydro-2H-benzo[1,4]oxazine-2-carboxylic acid (260 mg, 0.77 mmol) in DMF was added 4-(4-Fluoro-benzyl)-piperidine-4-carbonitrile (168 mg, 0.77 mmol), EDCI (220 mg, 1.15 m.mol), HOBt (125 mg, 0.92 m.mol) and DIPEA (297 mg, 2.23 m.mol) at 5-10° C. The reaction mixture was stirred at room temperature for 15 hours, diluted with water and extracted with ethyl acetate. The organic layer was washed with water, brine and dried over Na₂SO₄. The organic layer was concentrated and purified over column chromatography (Si-gel, 30% ethyl acetate-hexane) to afford 390 mg (93.8%) of 1-(6-Chloro-7-iodo-3,4-dihydro-2H-benzo[1,4]oxazine-2-carbonyl)-4-(4-fluoro-benzyl)-piperidine-4-carbonitrile. LC/MS [M+H]⁺: 540.2.

Step-4: 6-Chloro-2-[4-cyano-4-(4-fluoro-benzyl)-piperidine-1-carbonyl]-3,4-dihydro-2H-benzo[1,4]oxazine-7-carboxylic acid methyl ester

A solution of 1-(6-Chloro-7-iodo-3,4-dihydro-2H-benzo[1,4]oxazine-2-carbonyl)-4-(4-fluoro-benzyl)-piperidine-4-carbonitrile (100 mg, 0.19 mmol) in triethylamine-methanol (1:1) was degassed with argon during 30 mins. To the degassed solution were added Pd(OAc)2 (4 mg, 0.019 mmol) and Bis-1,3-(diphenylphosphino) propane (DPP) (15 mg, 0.037 mmol) in argon atmosphere. The reaction mixture was heated at 70° C. in presence of 220 psi carbon monoxide in a autoclave for overnight. The reaction mixture was cooled and filtered through celite bed. The methanolic solution was concentrated and purified through column chromatography (Si-gel, 40% ethyl acetate-hexane) to afford 33 mg (36.8%) of 6-Chloro-2-[4-cyano-4-(4-fluoro-benzyl)-piperidine-1-carbonyl]-3,4-dihydro-2H-benzo[1,4]oxazine-7-carboxylic acid methyl ester. LC/MS [M+H]⁺: 540.4.

Step-5: 6-Chloro-2-[4-cyano-4-(4-fluoro-benzyl)-piperidine-1-carbonyl]-3,4-dihydro-2H-benzo[1,4]oxazine-7-carboxylic acid methylamide

To a solution of 6-Chloro-2-[4-cyano-4-(4-fluoro-benzyl)-piperidine-1-carbonyl]-3,4-dihydro-2H-benzo[1,4]oxazine-7-carboxylic acid methyl ester (24 mg, 0.042 mmol) was dissolved in excess methylamine solution in THF and was heated at 90° C. in a sealed tube overnight. The reaction mixture was cooled and concentrated. The crude mass was purified through column chromatography (Si-gel, 2% MeoH-DCM) to afford 9 mg (45.6%) of 6-Chloro-2-[4-cyano-4-(4-fluoro-benzyl)-piperidine-1-carbonyl]-3,4-dihydro-2H-benzo[1,4]oxazine-7-carboxylic acid methylamide. LC/MS [M+H]⁺: 471.0. ¹H-NMR (400 MHz, DMSO-d₆) δ (ppm): 7.95 (m, 1H), 7.34 (m, 2H), 7.19 (m, 2H), 6.83 (m, 1H), 6.59 (s, 1H), 6.46 (bs, 1H), 4.95 (m, 1H), 4.38 (m, 1H), 4.06 (m, 1H), 3.43 (m, 1H), 3.16 (m, 1H), 3.00 (bs, 2H), 2.69 (bs, 3H), 1.89-1.53 (m, 5H). HPLC: 93.0%.

### Example 150

### 6-Chloro-2-[4-cyano-4-(4-fluoro-benzyl)-piperidine-1-carbonyl]-3,4-dihydro-2H-benzo[1,4]oxazine-7-carbonitrile

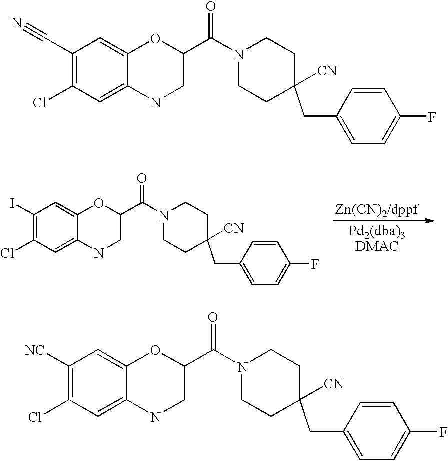

1-(6-Chloro-7-iodo-3,4-dihydro-2H-benzo[1,4]oxazine-2-carbonyl)-4-(4-fluoro-benzyl)-piperidine-4-carbonitrile was prepared by a process as described in example 149.

One drop of water was added to DMAC (1.5 ml) and degassed with argon for 1 hour orior to adding reactant. 1-(6-Chloro-7-iodo-3,4-dihydro-2H-benzo[1,4]oxazine-2-carbonyl)-4-(4-fluoro-benzyl)-piperidine-4-carbonitrile (100 mg, 0.18 mmol) was dissolved in DMAC at room temperature. To this solution was added Zn(CN)₂ (34 mg, 0.29 mmol), Pd₂(dba)₃ (8 mg, 0.009 mmol), dppf (9 mg, 0.067 mmol). The resulting mixture was heated at 120° C. overnight. The reaction mixture was diluted with water, extracted with ethyl acetate, washed with water, brine. The organic layer was dried over Na₂SO₄ and concentrated under reduced pressure. The crude mass was purified by column chromatography (silica gel, 0.5% MeOH-DCM) to get 28 mg (34.4%) 6-Chloro-2-[4-cyano-4-(4-fluoro-benzyl)-piperidine-1-carbonyl]-3,4-dihydro-2H-benzo[1,4]oxazine-7-carbonitrile. LC/MS [M+H]⁺: 439.6. ¹H-NMR (400 MHz, DMSO-d₆) δ (ppm): 7.24 (m, 6H), 6.73 (s, 1H), 5.03 (m, 1H), 4.36 (m, 1H), 4.07 (m, 1H), 3.51 (m, 1H), 3.31 (m, 1H), 3.15 (m, 2H), 2.95 (m, 2H), 2.67 (m, 1H), 1.80 (m, 3H). HPLC: 90.41%.

### Example 151

### 6-Chloro-2-[4-cyano-4-(4-fluoro-benzyl)-piperidine-1-carbonyl]-3,4-dihydro-2H-benzo[1,4]oxazine-7-carboxylic acid dimethylamide

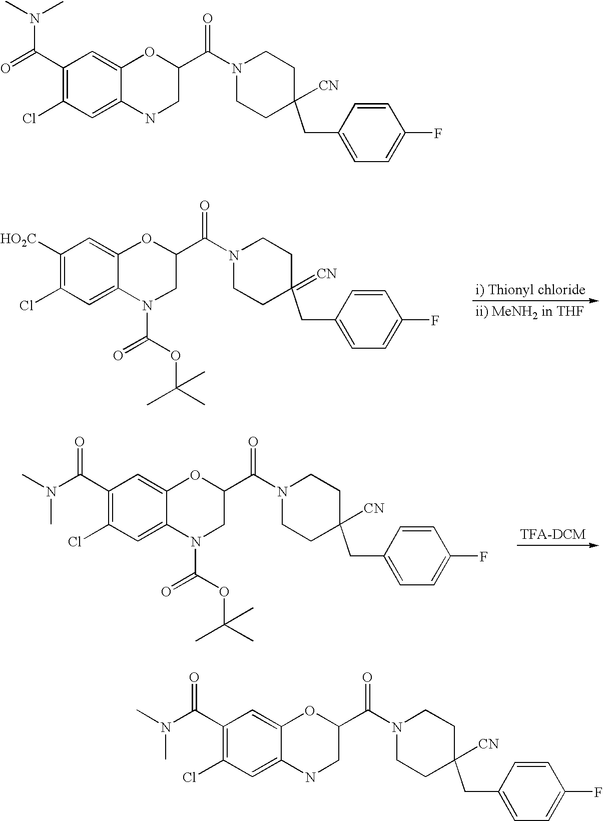

6-Chloro-2-[4-cyano-4-(4-fluoro-benzyl)-piperidine-1-carbonyl]-3,4-dihydro-2H-benzo[1,4]oxazine-7-carboxylic acid methyl ester was prepared by a process as described in example 149.

6-Chloro-2-[4-cyano-4-(4-fluoro-benzyl)-piperidine-1-carbonyl]-2,3-dihydro-benzo[1,4]oxazine-4,7-dicarboxylic acid 4-tert-butyl ester was prepared from 6-Chloro-2-[4-cyano-4-(4-fluoro-benzyl)-piperidine-1-carbonyl]-3,4-dihydro-2H-benzo[1,4]oxazine-7-carboxylic acid methyl ester by hydrolysis with LiOH.

Step-1: 6-Chloro-2-[4-cyano-4-(4-fluoro-benzyl)-piperidine-1-carbonyl]-3,4-dihydro-2H-benzo[1,4]oxazine-7-carboxylic acid dimethylamide

To a suspension of 6-Chloro-2-[4-cyano-4-(4-fluoro-benzyl)-piperidine-1-carbonyl]-2,3-dihydro-benzo[1,4]oxazine-4,7-dicarboxylic acid 4-tert-butyl ester (200 mg, 0.36 mmol) in dichloromethane was added thionyl chloride (2 ml) and hetaed at 50° C. The reaction mixture was concentrated under reduced pressure and tripped twice with toluene. The crude mass was stirred at room temperature with dimethylamine solution in THF (3 ml). The reaction mixture was concentrated and purified the crude mass through column chromatography (Si-gel, 2% MeOh-DCM) to afford 35 mg (16.7%) of 6-Chloro-2-[4-cyano-4-(4-fluoro-benzyl)-piperidine-1-carbonyl]-3,4-dihydro-2H-benzo[1,4]oxazine-7-carboxylic acid dimethylamide. LC/MS [M+H]⁺: 585.2.

Step-2: 6-Chloro-2-[4-cyano-4-(4-fluoro-benzyl)-piperidine-1-carbonyl]-3,4-dihydro-2H-benzo[1,4]oxazine-7-carboxylic acid dimethylamide

To a solution of 6-Chloro-2-[4-cyano-4-(4-fluoro-benzyl)-piperidine-1-carbonyl]-3,4-dihydro-2H-benzo[1,4]oxazine-7-carboxylic acid dimethylamide (35 mg, 0.06 mmol)in DCM was added trifluoroacetic acid (2 ml). The reaction mixture was stirred for about 12 h. The reaction mixture was concentrated and washed with dry ether. The resulting TFA salt was diluted with a minimum volume of water and neutralized with aq. NaOH solution at 5-10° C. Extraction of the neutralized solution with ethyl acetate, washed with water and brine solution. The organic layer was dried over Na₂SO₄ and concentrated under reduced pressure. The crude was purified through column chromatography (Si-gel, 2% MeOH-DCM) to afford 9 mg (30.9%) of 6-Chloro-2-[4-cyano-4-(4-fluoro-benzyl)-piperidine-1-carbonyl]-3,4-dihydro-2H-benzo[1,4]oxazine-7-carboxylic acid dimethylamide. LC/MS [M-H]⁺: 483.5. ¹H-NMR (400 MHz, DMSO-d₆) δ (ppm): 7.33 (m, 2H), 7.19 (m, 2H), 6.65 (bs, 1H), 6.62 (bs, 1H), 6.38 (m, 1H), 5.44 (m, 1H), 4.38 (m, 1H), 4.09 (m, 1H), 3.43 (m, 1H), 3.08 (m, 2H), 2.94 (s, 6H), 2.66 (s, 2H), 1.90-0.83 (m, 5H). HPLC: 95.1%.

### Example 152

### 1-[6-Chloro-7-(2-methyl-thiazol-4-yl)-3,4-dihydro-2H-benzo[1,4]oxazine-2-carbonyl]-4-(4-fluoro-benzyl)-piperidine-4-carbonitrile

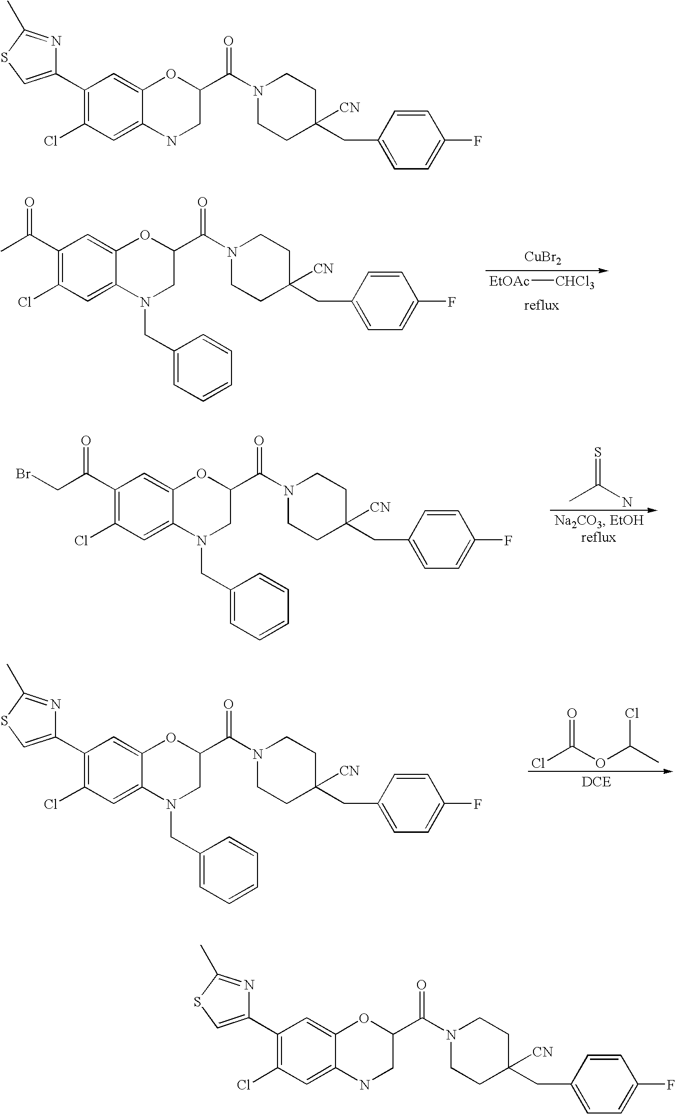

1-(7-Acetyl-4-benzyl-6-chloro-3,4-dihydro-2H-benzo[1,4]oxazine-2-carbonyl)-4-(4-fluoro-benzyl)-piperidine-4-carbonitrile was prepared by a process as described in example 145.
Step-1 : 1-[4-Benzyl-7-(2-bromo-acetyl)-6-chloro-3,4-dihydro-2H-benzo[1,4]oxazine-2-carbonyl]-4-(4-fluoro-benzyl)-piperidine-4-carbonitrile

1-(7-Acetyl-4-benzyl-6-chloro-3,4-dihydro-2H-benzo[1,4]oxazine-2-carbonyl)-4-(4-fluoro-benzyl)-piperidine-4-carbonitrile (200 mg, 0.37 mol) in CHCl3 (5 mL) was added to a solution of CuBr2 (200 mg, 0.88 mmol) in EtOAc (5 mL). The mixture was stirred for 12 h at reflux and then cooled to rt and filtered through Celite. The reaction mixture was concentrated. Purification by column chromatography (Si-gel, 40% ethyl acetate-hexane) gave 100 mg (43%) of 1-[4-Benzyl-7-(2-bromo-acetyl)-6-chloro-3,4-dihydro-2H-benzo[1,4]oxazine-2-carbonyl]-4-(4-fluoro-benzyl)-piperidine-4-carbonitrile. LC/MS [M+H]⁺: 62.6.2.

Step-2: 1-[4-Benzyl-6-chloro-7-(2-methyl-thiazol-4-yl)-3,4-dihydro-2H-benzo[1,4]oxazine-2-carbonyl]-4-(4-fluoro-benzyl)-piperidine-4-carbonitrile

To a solution of 1-[4-Benzyl-7-(2-bromo-acetyl)-6-chloro-3,4-dihydro-2H-benzo[1,4]oxazine-2-carbonyl]-4-(4-fluoro-benzyl)-piperidine-4-carbonitrile (100 mg, 0.16 mmol) in ethanol was added thoacetamide (15 mg, 0.19 mmol) and Na₂CO₃ (26 mg, 0.24 mmol). The reaction mixture was refluxed for 6 h. The reaction mixture was concentrated, diluted with water and extracted with ethyl acetate. The organic layer was washed with water, brine and dried over Na₂SO₄. The organic layer was concentrated under reduced pressure to afford 45 mg (46.8%) of 1-[4-Benzyl-6-chloro-7-(2-methyl-thiazol-4-yl)-3,4-dihydro-2H-benzo[1,4]oxazine-2-carbonyl]-4-(4-fluoro-benzyl)-piperidine-4-carbonitrile. LC/MS [M+H]⁺: 601.2.

Step-3: 1-[6-Chloro-7-(2-methyl-thiazol-4-yl)-3,4-dihydro-2H-benzo[1,4]oxazine-2-carbonyl]4-(4-fluoro-benzyl)-piperidine-4-carbonitrile

To a solution of 1-[4-Benzyl-6-chloro-7-(2-methyl-thiazol-4-yl)-3,4-dihydro-2H-benzo[1,4]oxazine-2-carbonyl]-4-(4-fluoro-benzyl)-piperidine-4-carbonitrile (22 mg, 0.037 mmol) in dichloroethane was added 1-chloroethylchloroformate (0.1 ml). The reaction mixture was refluxed for 6 h and methanol (10 ml) was added. The reaction mixture was refluxed again for another 3 h. The reaction mixture was concentrated and purified through column chromatography (Si-gel, 50% ethyl acetate-hexane) afford 6 mg (31.7%) of 1-[6-Chloro-7-(2-methyl-thiazol-4-yl)-3,4-dihydro-2H-benzo[1,4]oxazine-2-carbonyl]-4-(4-fluoro-benzyl)-piperidine-4-carbonitrile.

LC/MS [M+H]⁺: 511.2. HPLC: 87.2%.

### Human CCR1 Binding Assay (Ki Determination in 96 Well Format)

### Competition-Equilibrium Binding Assay

After trypsinization, CHO-K1-Gα16 (CHO-K1 from ATCC, Gα16 cloned and expressed in CHO-K 1 cells in CBT) cells expressing human wild-type CCR1 were harvested using calcium and magnesium free HBSS (INVITROGEN) buffer and then resuspended at 400,000 cells/ mL in binding buffer (RPMI1640, pH 7.4, 0.2% BSA, Complete protease inhibitor cocktail without EDTA from Roche) Competition-binding assays were performed in 96 well plates (Corning non binding surface flat clear bottom white 96 well), using 25 μL of cell suspension (10,000/well), 25 μL/ well of 100 μM solution of [125I]-RANTES (2200 Ci/mmol; Perkin Elmer NEX292) as tracer for human CCR1 receptor, 25 μL/ well of RPMI buffer or various concentration of competitor/compound diluted in RPMI and 25 μL/ well of PVT-WGA SPA beads (GE) (0.1 mg/well). Each well contained a final volume of 100 μL with a final DMSO concentration of 0.5%. The 96 well plates were incubated for 2 hours at room temperature in the dark without shaking. Bound radioligand was counted with a beta scintillation counter (Wallac, Micro-Beta Trilux counter) for 1 min per well. Total binding of [125I]-RANTES was measured in the absence and presence of competitor (compound), the nonspecific binding was measured with a 33-fold excess of BX471 (10 μM final). In order to determine binding parameters (Ki and IC₅₀), the counts results were analyzed using the one-site competition curve fitting functions in GraphPad PRISM, v. 4.0 (San Diego, Calif.).

The compounds of the present invention typically exhibit potency values of greater than 50% inhibition at 20 μM in this competitive binding assay.

The entire disclosures of all applications, patents and publications, cited above and below, are hereby incorporated by reference.

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

What is claimed is:

1. A compound having the chemical structure: 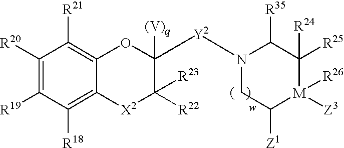
wherein
X² is 0 or —NR¹⁷, where R¹⁷ is hydrogen, alkyl, acyl, heteroaryl, —C(O)—CF₃, cyano, alkylsulfinyl, alkylsulfonyl, amino, —C(O)-aminoalkyl, -alkyleneamino, -alkylene-OH, -alkylene-C(O)NR^{e}R^{f}, or -alkylene-NR^{e}R^{f}, where R^{e} and R^{f} are each, independently, hydrogen or alkyl;
y² is —C(O)—, —CH₂C(O)—, —CH₂CH₂— or —CH₂—;
M is selected from the group consisting of —C—, —N—, and —O—;
Z¹ is selected from the group consisting of hydrogen, aryl, heteroaryl, heterocycle, arylalkyl, heteroarylalkyl or heterocyclealkyl;
Z³ is —C(O)-aryl, —C(O)-heteoraryl, —C(O)-arylalkyl, or —C(O)-heteroarylalkyl, arylalkyl or heteroarylalkyl;
R¹⁸, R¹⁹, R²⁰ and R²¹ are each, independently, hydrogen, halogen, hydroxy, cyano, nitro, amino, alkylamino, dialkylamino, carboxyl, alkyl, alkenyl, alkynyl, cycloalkyl, cycloalkylalkyl, aryl, arylalkyl, heteroaryl, heteroarylamino, heteroarylalkyl, heterocycle, heterocyclealkyl, aroyl, acyl, alkoxy, aryloxy, cycloalkyloxy, cycloalkylalkyloxy, arylalkyloxy, heteroarylalkyloxy, alkythio, arylthio, alkylsulfinyl, alkylsulfonyl, alkoxycarbonyl, aryloxycarbonyl, heteroaryloxycarbonyl —O-alkylene-CO₂H, —O-alkylene-C(O)NR^{x}R^{y} or NR^{x}C(O)-alkyl, in which R^{x} and R^{y} are each, independently, hydrogen or alkyl;
R²² and R²³ are each hydrogen, or R²² and R²³, together with the carbon atom to which they are attached, form a —C(O)-group;
R²⁴ and R²⁵ are each, independently, hydrogen, alkyl or arylalkyl;
V is alkyl;
w is 0, 1 or 2;
q is 0 or 1;
R²⁶ is hydrogen, hydroxyl, —CO₂Et, cyano, heteroaryl, -alkylene-OH, or -alkylene-NH₂; wherein
when R²⁶ is hydrogen, then X² is —NR¹⁷— and Z³ is —C(O)-aryl, —C(O)-heteoraryl, —C(O)-arylalkyl, or —C(O)-heteroarylalkyl; and
when R²⁶ is hydroxyl, —CO₂Et, cyano, heteroaryl, -alkylene-OH, or -alkylene-NH₂, then Z³ is arylalkyl or heteroarylalkyl;
R³⁵ is hydrogen or alkyl;
provided, however, that when R²⁶ is present, then M is —C—;
provided, however, that when Z³ is present, then M is —C— or —N—;
provided, however, than when M is —O—, then Z¹ is arylalkyl;
provided, however, than when w is 1, X² is —O—, and M is —N—, then Z³ is optionally substituted 4-fluorobenzyl,
wherein, when present, any aryl, heteroaryl or heterocycle group may optionally be substituted by halogen, hydroxy, cyano, nitro, amino, alkylamino, dialkylamino, arylamino, diarylamino, amido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, and combinations thereof;
and pharmaceutically acceptable salts or solvates or N-oxides thereof, or solvates of pharmaceutically acceptable salts thereof, or pharmaceutically acceptable salts or solvates of N-oxides thereof, prodrugs and isomers thereof,
with the proviso that said compound is not:
[4-[(2-chloro-4-fluorophenyl)methyl]-1-piperazinyl](2,3-dihydro-1,4-benzodioxin-2-yl)-methanone,
1-[2,3-dihydro-2-[[4-(1-naphthalenylmethyl)-1-piperazinyl]carbonyl]-4H-1,4-benzoxazin-4-yl]-ethanone,
1-[2,3-dihydro-2-[[4-[[4-(trifluoromethyl)phenyl]methyl]-1-piperazinyl]carbonyl]4H-1,4-benzoxazin-4-yl]-ethanone,
[4-(ethylsulfonyl)-3,4-dihydro-2H-1,4-benzoxazin-2-yl][4-(phenylmethyl)-1-piperazinyl]-methanone,
2-[2-oxo-2-[4-(phenylmethyl)-1-piperazinyl]ethyl]-2H-1,4,-benzoxazin-3(4H)-one, or
1-2[[hexahydro-4-[(4-methoxyphenyl)methyl]-1H-1,4-diazepin-1-yl]carbonyl]-2,3-dihydro-4H-1,4-benzoxazin-4-yl]-ethanone,
1-[2-[(4-benzoyl-1-piperidinyl)carbonyl]-2,3-dihydro-4H-1,4-benzoxazin-4-yl]-ethanone,
1-[2-[(4-(4-hydroxy)benzoyl-1-piperidinyl)carbonyl]-2,3-dihydro-4H-1,4-benzoxazin-4-yl]-ethanone,
1-[2-(2,3-dihydro-1,4-benzodioxin-2-yl)ethyl]-4-(4-methylphenyl)-4-piperidinol,
or a pharmaceutically acceptable salt thereof.

2. A compound according to claim 1, selected from:
(6-Chloro-2,3-dihydro-benzo[1,4]dioxin-2-yl)-[(R)-4-(4-fluoro-benzyl)-2-methyl-piperazin-1-yl]-methanone,
(2,3-Dihydro-benzo[1,4]dioxin-2-yl)-[(R)-4-(4-fluoro-benzyl)-2-methyl-piperazin-1-yl]-methanone,
(2,3-Dihydro-benzo[1,4]dioxin-2-yl)-[4-(4-fluoro-benzyl)-piperazin-1-yl]-methanone,
(6-Chloro-2,3-dihydro-benzo[1,4]dioxin-2-yl)-[4-(4-fluoro-benzyl)-piperazin-1-yl]-methanone,
(6-Chloro-2,3-dihydro-benzo[1,4]dioxin-2-yl)-[4-(4-fluoro-benzyl)-[1,4]diazepan-1-yl]-methanone,
(6,7-Dichloro-2,3-dihydro-benzo[1,4]dioxin-2-yl)-[(R)-4-(4-fluoro-benzyl)-2-methyl-piperazin-1-yl]-methanone,
(6,7-Dichloro-2,3-dihydro-benzo[1,4]dioxin-2-yl)-[4-(4-fluoro-benzyl)-[1,4]diazepan-1-yl]-methanone,
(6-Chloro-3,4-dihydro-2H-benzo[1,4]oxazin-2-yl)-[(R)-4-(4-fluoro-benzyl)-2-methyl-piperazin-1-yl]-methanone,
(6-Chloro-3,4-dihydro-2H-benzo[1,4]oxazin-2-yl)-[4-(4-fluoro-benzyl)-piperazin-1-yl]-methanone,
(6-Chloro-3,4-dihydro-2H-benzo[1,4]oxazin-2-yl)-[4-(4-fluoro-benzyl)-[1,4]diazepan-1-yl]-methanone,
6-Chloro-2-[4-(4-fluoro-benzyl)-[1,4]diazepane-1-carbonyl]-2,3-dihydro-benzo[1,4]oxazine-4-carboxylic acid amide,
(6-Chloro-4-methyl-3,4-dihydro-2H-benzo[1,4]oxazin-2-yl)-[(R)-4-(4-fluoro-benzyl)-2-methyl-piperazin-1-yl]-methanone,
(6-Chloro-4-methyl-3,4-dihydro-2H-benzo[1,4]oxazin-2-yl)-[4-(4-fluoro-benzyl)-[1,4]diazepan-1-yl]-methanone,
6-Chloro-2-[(R)-4-(4-fluoro-benzyl)-2-methyl-piperazine-1-carbonyl]-4H-benzo[1,4]oxazin-3-one,
1-{6-Chloro-2-[4-(4-fluoro-benzyl)-piperazine-1-carbonyl]-2,3-dihydro-benzo[1,4]oxazin-4-yl}-ethanone,
(6-Chloro-4-methanesulfonyl-3,4-dihydro-2H-benzo[1,4]oxazin-2-yl)-[4-(4-fluoro-benzyl)-piperazin-1-yl]-methanone,
(6-Chloro-3,4-dihydro-2H-benzo[1,4]oxazin-2-yl)-[7-(4-fluoro-benzyl)-[1,4]oxazepan-4-yl]methanone,
1-{6-Chloro-2-[4-(4-fluoro-benzyl)-piperazine-1-carbonyl]-2,3-dihydro-benzo[1,4]oxazin-4-yl}-2-dimethylamino-ethanone,
(6-Chloro-4-methyl-3,4-dihydro-2H-benzo[1,4]oxazin-2-yl)-[4-(4-fluoro-benzyl)-piperazin-1-yl]-methanone,
1-{6-Chloro-2-[4-(4-fluoro-benzyl)-piperazine-1-carbonyl]-2,3-dihydro-benzo[1,4]oxazin-4-yl}-2-methylamino-ethanone,
2-(6-Chloro-3,4-dihydro-2H-benzo[1,4]oxazin-2-yl)-1-[(R)-4-(4-fluoro-benzyl)-2-methyl-piperazin-1-yl]-ethanone,
2-(6-Chloro-3,4-dihydro-2H-benzo[1,4]oxazin-2-yl)-1-[4-(4-fluoro-benzyl)-[1,4]diazepan-1-yl]ethanone,
(6-Chloro-7-methoxy-3,4-dihydro-2H-benzo[1,4]oxazin-2-yl)-[(R)-4-(4-fluoro-benzyl)-2-methyl-piperazin-1-yl]-methanone,
(6-Chloro-7-methoxy-4-methyl-3,4-dihydro-2H-benzo[1,4]oxazin-2-yl)-[(R)-4-(4-fluoro-benzyl)-2-methyl-piperazin-1-yl]-methanone,
(6-Chloro-3,4-dihydro-2H-benzo[1,4]oxazin-2-yl)-[7-(4-fluoro-benzyl)-[1,4]oxazepan-4-yl]-methanone,
2-(6-Chloro-3,4-dihydro-2H-benzo[1,4]oxazin-2-yl)-1-[7-(4-fluoro-benzyl)-[1,4]oxazepan-4-yl]-ethanone,
2-(6-Chloro-3,4-dihydro-2H-benzo[1,4]oxazin-2-yl)-1-[2-(4-fluoro-benzyl)-[1,4]oxazepan-4-yl]-ethanone, and
6-Chloro-2-{2-[2-(4-fluoro-benzyl)-[1,4]oxazepan-4-yl]-ethyl}-3,4-dihydro-2H-benzo[1,4]oxazine,
(6-Chloro-3,4-dihydro-2H-benzo[1,4]oxazin-2-yl)-[4-(4-fluoro-benzoyl)-piperidin-1-yl]-methanone,
1-(6-Chloro-3,4-dihydro-2H-benzo[1,4]oxazine-2-carbonyl)-4-(4-fluoro-benzyl)-piperidine-4-carbonitrile,
[1-(6-Chloro-3,4-dihydro-2H-benzo[1,4]oxazin-2-ylmethyl)-4-(4-fluoro-benzyl)-piperidin-4-yl]-methylamine,
1-(6-Chloro-7-methoxy-3,4-dihydro-2H-benzo[1,4]oxazine-2-carbonyl)-4-(4-fluoro-benzyl)-piperidine-4-carbonitrile,
4-(4-Chloro-benzyl)-1-(6-chloro-3,4-dihydro-2H-benzo[1,4]oxazine-2-carbonyl)-piperidine-4-carbonitrile,
[4-(4-Chloro-benzyl)-4-hydroxy-3,3-dimethyl-piperidin-1-yl]-(6-chloro-3,4-dihydro-2H-benzo[1,4]oxazin-2-yl)-methanone,
4-(4-Chloro-benzyl)-1-(6-chloro-3,4-dihydro-2H-benzo[1,4]oxazin-2-ylmethyl)-3,3-dimethyl-piperidin-4-ol,
1-(6-Chloro-7-methoxy-4-methyl-3,4-dihydro-2H-benzo[1,4]oxazine-2-carbonyl)-4-(4-fluoro-benzyl)-piperidine-4-carbonitrile,
1-(6-Chloro-7-hydroxy-3,4-dihydro-2H-benzo[1,4]oxazine-2-carbonyl)-4-(4-fluoro-benzyl)-piperidine-4-carbonitrile,
2-{6-Chloro-2-[4-cyano-4-(4-fluoro-benzyl)-piperidine-1-carbonyl]-3,4-dihydro-2H-benzo[1,4]oxazin-7-yloxy}-acetic acid,
2-{6-Chloro-2-[4-cyano-4-(4-fluoro-benzyl)-piperidine-1-carbonyl]-3,4-dihydro-2H-benzo[1,4]oxazin-7-yloxy}-acetamide,
2-{6-Chloro-2-[4-cyano-4-(4-fluoro-benzyl)-piperidine-1-carbonyl]-3,4-dihydro-2H-benzo[1,4]oxazin-7-yloxy}-N,N-dimethyl-acetamide,
1-(6-Chloro-7-ethoxy-3,4-dihydro-2H-benzo[1,4]oxazine-2-carbonyl)-4-(4-fluoro-benzyl)-piperidine-4-carbonitrile,
1-[6-Chloro-4-(2-dimethylamino-acetyl)-7-methoxy-3,4-dihydro-2H-benzo[1,4]oxazine-2-carbonyl]-4-(4-fluoro-benzyl)-piperidine-4-carbonitrile,
1-(4-Acetyl-6-chloro-7-methoxy-3,4-dihydro-2H-benzo[1,4]oxazine-2-carbonyl)-4-(4-fluoro-benzyl)-piperidine-4-carbonitrile,
1-[4-(4-Chloro-benzyl)-4-hydroxy-3,3-dimethyl-piperidin-1-yl]-2-(6-chloro-3,4-dihydro-2H-benzo[1,4]oxazin-2-yl)-ethanone,
4-(4-Chloro-benzyl)-1-[2-(6-chloro-3,4-dihydro-2H-benzo[1,4]oxazin-2-yl)-ethyl]-3,3-dimethyl-piperidin-4-ol,
1-[2-(6-Chloro-3,4-dihydro-2H-benzo[1,4]oxazin-2-yl)-ethyl]-4-(4-fluoro-benzyl)-3,3-dimethyl-piperidin-4-ol,
1-(6-Chloro-4-methanesulfonyl-7-methoxy-3,4-dihydro-2H-benzo[1,4]oxazine-2-carbonyl)-4-(4-fluoro-benzyl)-piperidine-4-carbonitrile,
2-{6-Chloro-2-[4-cyano-4-(4-fluoro-benzyl)-piperidine-1-carbonyl]-7-methoxy-2,3-dihydro-benzo[1,4]oxazin-4-yl) -acetamide,
1-(6-Chloro-7-dimethylamino-3,4-dihydro-2H-benzo[1,4]oxazine-2-carbonyl)-4-(4-fluoro-benzyl)-piperidine-4-carbonitrile,
N-{6-Chloro-2-[4-cyano-4-(4-fluoro-benzyl)-piperidine-1-carbonyl]-3,4-dihydro-2H-benzo[1,4]oxazin-7-yl) -acetamide,
4-(4-Fluoro-benzyl)-1-(7-methoxy-3,4-dihydro-2H-benzo[1,4]oxazine-2-carbonyl)-piperidine-4-carbonitrile,
6-Chloro-2-[4-cyano-4-(4-fluoro-benzyl)-piperidine-1-carbonyl]-7-methoxy-2,3-dihydro-benzo[1,4]oxazine-4-carboxylic acid amide,
1-(7-Bromo-6-chloro-3,4-dihydro-2H-benzo[1,4]oxazine-2-carbonyl)-4-(4-fluoro-benzyl)-piperidine-4-carbonitrile,
1-(6-Chloro-8-dimethylamino-3,4-dihydro-2H-benzo[1,4]oxazine-2-carbonyl)-4-(4-fluoro-benzyl)-piperidine-4-carbonitrile,
1-(6,7-Dichloro-3,4-dihydro-2H-benzo[1,4]oxazine-2-carbonyl)-4-(4-fluoro-benzyl)-piperidine-4-carbonitrile,
1-(6-Chloro-7-isopropoxy-3,4-dihydro-2H-benzo[1,4]oxazine-2-carbonyl)-4-(4-fluoro-benzyl)-piperidine-4-carbonitrile,
1-(4-Acetyl-6-chloro-7-dimethylamino-3,4-dihydro-2H-benzo[1,4]oxazine-2-carbonyl)-4-(4-fluoro-benzyl)-piperidine-4-carbonitrile,
2-{6-Chloro-2-[4-cyano-4-(4-fluoro-benzyl)-piperidine-1-carbonyl]-7-dimethylamino-2,3-dihydro-benzo[1,4]oxazin-4-yl}-acetamide,
1-(6-Chloro-7-dimethylamino-4-methanesulfonyl-3,4-dihydro-2H-benzo[1,4]oxazine-2-carbonyl)-4-(4-fluoro-benzyl)-piperidine-4-carbonitrile,
2-{6-Chloro-2-[4-cyano-4-(4-fluoro-benzyl)-piperidine-1-carbonyl]-7-isopropoxy-2,3-dihydro-benzo[1,4]oxazin-4-yl}-acetamide,
2-{6-Chloro-2-[4-cyano-4-(4-fluoro-benzyl)-piperidine-1-carbonyl]-7-ethoxy-2,3-dihydro-benzo[1,4]oxazin-4-yl}-acetamide,
1-(6-Chloro-7-ethoxy-4-methanesulfonyl-3,4-dihydro-2H-benzo[1,4]oxazine-2-carbonyl)-4-(4-fluoro-benzyl)-piperidine-4-carbonitrile,
1-(6-Chloro-7-pyrrolidin-1-yl-3,4-dihydro-2H-benzo[1,4]oxazine-2-carbonyl)-4-(4-fluoro-benzyl)-piperidine-4-carbonitrile,
1-(7-Azetidin-1-yl-6-chloro-3,4-dihydro-2H-benzo[1,4]oxazine-2-carbonyl)-4-(4-fluoro-benzyl)-piperidine-4-carbonitrile,
1-(6-Chloro-7-difluoromethoxy-3,4-dihydro-2H-benzo[1,4]oxazine-2-carbonyl)-4-(4-fluoro-benzyl)-piperidine-4-carbonitrile,
1-(7-Chloro-6-methoxy-3,4-dihydro-2H-benzo[1,4]oxazine-2-carbonyl)-4-(4-fluoro-benzyl)-piperidine-4-carbonitrile,
1-(6-Chloro-7-diethylamino-3,4-dihydro-2H-benzo[1,4]oxazine-2-carbonyl)-4-(4-fluoro-benzyl)-piperidine-4-carbonitrile,
1-(6-Chloro-7-morpholin-4-yl-3,4-dihydro-2H-benzo[1,4]oxazine-2-carbonyl)-4-(4-fluoro-benzyl)-piperidine-4-carbonitrile,
(6-Chloro-7-methoxy-3,4-dihydro-2H-benzo[1,4]oxazin-2-yl)-[4-(4-fluoro-phenoxy)-piperidin-1-yl]-methanone,
1-[6-Chloro-4-(2-hydroxy-ethyl)-7-methoxy-3,4-dihydro-2H-benzo[1,4]oxazine-2-carbonyl]-4-(4-fluoro-benzyl)-piperidine-4-carbonitrile,
1-[6-Chloro-7-dimethylamino-4-(2-hydroxy-ethyl)-3,4-dihydro-2H-benzo[1,4]oxazine-2-carbonyl]-4-(4-fluoro-benzyl)-piperidine-4-carbonitrile,
6-Chloro-2-[4-cyano-4-(4-fluoro-benzyl)-piperidine-1-carbonyl]-7-dimethylamino-2,3-dihydro-benzo[1,4]oxazine-4-carbonitrile,
1-(6-Chloro-7-dimethylamino-3,4-dihydro-2H-benzo[1,4]oxazine-2-carbonyl)-4-(6-fluoro-pyridin-3-ylmethyl)-piperidine-4-carbonitrile,
1-(6-Chloro-7-methylamino-3,4-dihydro-2H-benzo[1,4]oxazine-2-carbonyl)-4-(4-fluoro-benzyl)-piperidine-4-carbonitrile,
2-{6-Chloro-2-[4-cyano-4-(4-fluoro-benzyl)-piperidine-1-carbonyl]-7-methoxy-2,3-dihydro-benzo[1,4]oxazin-4-yl}-N-methyl-acetamide,
1-(6-Chloro-7-methoxy-3,4-dihydro-2H-benzo[1,4]oxazine-2-carbonyl)-4-(6-fluoro-pyridin-3-ylmethyl)-piperidine-4-carbonitrile,
6-Chloro-2-[4-cyano-4-(4-fluoro-benzyl)-piperidine-1-carbonyl]-7-methoxy-2,3-dihydro-benzo[1,4]oxazine-4-carbonitrile,
1-(6-Chloro-7-methoxy-3,4-dihydro-2H-benzo[1,4]oxazine-2-carbonyl)-4-(4-fluoro-benzyl)-piperidine-4-carboxylic acid ethyl ester,
1-(6-Chloro-7-dimethylamino-3,4-dihydro-2H-benzo[1,4]oxazine-2-carbonyl)-4-(4-fluoro-benzyl)-piperidine-4-carboxylic acid ethyl ester,
1-(6-Chloro-7-methoxy-3,4-dihydro-2H-benzo[1,4]oxazine-2-carbonyl)-4-pyridin-4-ylmethyl-piperidine-4-carbonitrile,
1-(6-Chloro-7-dimethylamino-3,4-dihydro-2H-benzo[1,4]oxazine-2-carbonyl)-4-pyridin-4-ylmethyl-piperidine-4-carbonitrile,
(6-Chloro-7-methoxy-3,4-dihydro-2H-benzo[1,4]oxazin-2-yl)-[4-(4-fluoro-benzyl)-4-hydroxymethyl-piperidin-1-yl]-methanone,
(6-Chloro-7-dimethylamino-3,4-dihydro-2H-benzo[1,4]oxazin-2-yl)-[4-(4-fluoro-benzyl)-4-hydroxymethyl-piperidin-1-yl]-methanone,
1-(6-Chloro-7-dimethylamino-3,4-dihydro-2H-benzo[1,4]oxazine-2-carbonyl)-4-[(4-fluoro-phenyl)-hydroxy-methyl]-piperidine-4-carbonitrile,
1-[6-Chloro-7-dimethylamino-4-(2-dimethylamino-ethyl)-3,4-dihydro-2H-benzo[1,4]oxazine-2-carbonyl]-4-(4-fluoro-benzyl)-piperidine-4-carbonitrile,
1-[6-Chloro-4-(2-dimethylamino-ethyl)-7-methoxy-3,4-dihydro-2H-benzo[1,4]oxazine-2-carbonyl]-4-(4-fluoro-benzyl)-piperidine-4-carbonitrile,
(6-Chloro-7-methoxy-3,4-dihydro-2H-benzo[1,4]oxazin-2-yl)-[4-(4-fluoro-benzyl)-4-imidazol-1-ylmethyl-piperidin-1-yl]-methanone,
1-(6-Chloro-7-ethyl-3,4-dihydro-2H-benzo[1,4]oxazine-2-carbonyl)-4-(4-fluoro-benzyl)-piperidine-4-carbonitrile,
1-(6-Chloro-7-methoxy-3,4-dihydro-2H-benzo[1,4]oxazine-2-carbonyl)-4-[(4-fluoro-phenyl)-hydroxy-methyl]-piperidine-4-carbonitrile,
1-(6-Chloro-7-dimethylamino-3,4-dihydro-2H-benzo[1,4]oxazine-2-carbonyl)-3-(4-fluoro-benzyl)-pyrrolidine-3-carbonitrile,
{6-Chloro-2-[4-cyano-4-(4-fluoro-benzyl)-piperidine-1-carbonyl]-3,4-dihydro-2H-benzo[1,4]oxazin-7-yl}-methyl-cyanamide,
1-(6-Chloro-7-methoxy-3,4-dihydro-2H-benzo[1,4]oxazine-2-carbonyl)-4-[difluoro-(4-fluoro-phenyl)-methyl]-piperidine-4-carbonitrile,
1-(6-Chloro-7-dimethylamino-3,4-dihydro-2H-benzo[1,4]oxazine-2-carbonyl)-4-[difluoro-(4-fluoro-phenyl)-methyl]-piperidine-4-carbonitrile,
1-[6-Chloro-7-(1H-pyrazol-4-yl)-3,4-dihydro-2H-benzo[1,4]oxazine-2-carbonyl]-4-(4-fluoro-benzyl)-piperidine-4-carbonitrile,
6-Chloro-7-[methyl-(2H-tetrazol-5-yl)-amino]-3,4-dihydro-2H-benzo[1,4]oxazine-2-carboxylic acid [3-cyano-3-(4-fluoro-benzyl)-pentyl]-methyl-amide,
1-[6-Chloro-7-(2H-tetrazol-5-ylamino)-3,4-dihydro-2H-benzo[1,4]oxazine-2-carbonyl]-4-(4-fluoro-benzyl)-piperidine-4-carbonitrile,
1-[6-Chloro-7-dimethylamino-4-(2,2,2-trifluoro-acetyl)-3,4-dihydro-2H-benzo[1,4]oxazine-2-carbonyl]-4-(4-fluoro-benzyl)-piperidine-4-carbonitrile,
1-[6-Chloro-7-methoxy-4-(2,2,2-trifluoro-acetyl)-3,4-dihydro-2H-benzo[1,4]oxazine-2-carbonyl]-4-(4-fluoro-benzyl)-piperidine-4-carbonitrile,
1-(6-Chloro-7-methoxy-3-oxo-3,4-dihydro-2H-benzo[1,4]oxazine-2-carbonyl)-4-(4-fluoro-benzyl)-piperidine-4-carbonitrile,
1-(6-Chloro-7-imidazol-1-yl-3,4-dihydro-2H-benzo[1,4]oxazine-2-carbonyl)-4-(4-fluoro-benzyl)-piperidine-4-carbonitrile,
1-[6-Chloro-7-methoxy-4-(2H-tetrazol-5-yl)-3,4-dihydro-2H-benzo[1,4]oxazine-2-carbonyl]-4-(4-fluoro-benzyl)-piperidine-4-carbonitrile,
4-(4-Fluoro-benzyl)-1-(quinoxaline-2-carbonyl)-piperidine-4-carbonitrile,
4-(4-Fluoro-benzyl)-1-([1,2,4]triazolo[1,5-a]pyrimidine-2-carbonyl)-piperidine-4-carbonitrile,
4-(4-Fluoro-benzyl)-1-(pyrazolo[1,5-a]pyrimidine-3-carbonyl)-piperidine-4-carbonitrile
6-Chloro-2-[4-cyano-4-(4-fluoro-benzyl)-piperidine-1-carbonyl]-2,3-dihydro-benzo[1,4]oxazine-4-carbonitrile,
1-[6-Chloro-4-(2H-tetrazol-5-yl)-3,4-dihydro-2H-benzo[1,4]oxazine-2-carbonyl]-4-(4-fluoro-benzyl)-piperidine-4-carbonitrile,
1-[6-Chloro-4-(1H-[1,2,4]triazol-3-yl)-3,4-dihydro-2H-benzo[1,4]oxazine-2-carbonyl]-4-(4-fluoro-benzyl)-piperidine-4-carbonitrile,
1-(6-Chloro-7-pyrrol-1-yl-3,4-dihydro-2H-benzo[1,4]oxazine-2-carbonyl)-4-(4-fluoro-benzyl)piperidine-4-carbonitrile,
1-[6-Chloro-7-methoxy-4-(1H-[1,2,4]triazol-3-yl)-3,4-dihydro-2H-benzo[1,4]oxazine-2-carbonyl]-4-(4-fluoro-benzyl)-piperidine-4-carbonitrile,
1-[6-Chloro-8-(1H-pyrazol-4-yl)-3,4-dihydro-2H-benzo[1,4]oxazine-2-carbonyl]-4-(4-fluoro-benzyl)-piperidine-4-carbonitrile,
1-(6-Chloro-8-pyridin-4-yl-3,4-dihydro-2H-benzo[1,4]oxazine-2-carbonyl)-4-(4-fluoro-benzyl)-piperidine-4-carbonitrile,
1-(6-Chloro-8-thiophen-3-yl-3,4-dihydro-2H-benzo[1,4]oxazine-2-carbonyl)-4-(4-fluoro-benzyl)-piperidine-4-carbonitrile,
1-(6-Chloro-7-thiophen-3-yl-3,4-dihydro-2H-benzo[1,4]oxazine-2-carbonyl)-4-(4-fluoro-benzyl)-piperidine-4-carbonitrile,
1-(6-Chloro-7-pyridin-3-yl-3,4-dihydro-2H-benzo[1,4]oxazine-2-carbonyl)-4-(4-fluoro-benzyl)-piperidine-4-carbonitrile,
1-(6-Chloro-7-pyridin-4-yl-3,4-dihydro-2H-benzo[1,4]oxazine-2-carbonyl)-4-(4-fluoro-benzyl)-piperidine-4-carbonitrile,
1-[6-Chloro-7-(2-methyl-2H-pyrazol-3-yl)-3,4-dihydro-2H-benzo[1,4]oxazine-2-carbonyl]-4-(4-fluoro-benzyl)-piperidine-4-carbonitrile,
1-[6-Chloro-7-(1-methyl-1H-pyrazol-4-yl)-3,4-dihydro-2H-benzo[1,4]oxazine-2-carbonyl]-4-(4-fluoro-benzyl)-piperidine-4-carbonitrile,
1-(6-Chloro-7-dimethylamino-3,4-dihydro-2H-benzo[1,4]oxazine-2-carbonyl)-4-(4-fluoro-benzyl)-piperidine-4-carbonitrile,
1-(6-Chloro-7-pyrazol-1-yl-3,4-dihydro-2H-benzo[1,4]oxazine-2-carbonyl)-4-(4-fluoro-benzyl)-piperidine-4-carbonitrile,
1-(6-Chloro-7-pyrimidin-5-yl-3,4-dihydro-2H-benzo[1,4]oxazine-2-carbonyl)-4-(4-fluoro-benzyl)-piperidine-4-carbonitrile,
1-(6-Chloro-7-hexa deuterio dimethylamino-3,4-dihydro-2H-benzo[1,4]oxazine-2-carbonyl)-4-(4-fluoro-benzyl)-piperidine-4-carbonitrile,
1-(6-Chloro-7-furan-2-yl-3,4-dihydro-2H-benzo[1,4]oxazine-2-carbonyl)-4-(4-fluoro-benzyl)-piperidine-4-carbonitrile,
1-(6-Chloro-7-furan-3-yl-3,4-dihydro-2H-benzo[1,4]oxazine-2-carbonyl)-4-(4-fluoro-benzyl)-piperidine-4-carbonitrile,
1-[6-Chloro-7-(4-methyl-imidazol-1-yl)-3,4-dihydro-2H-benzo[1,4]oxazine-2-carbonyl]-4-(4-fluoro-benzyl)-piperidine-4-carbonitrile,
1-[6-Chloro-7-(3-methyl-pyrazol-1-yl)-3,4-dihydro-2H-benzo[1,4]oxazine-2-carbonyl]-4-(4-fluoro-benzyl)-piperidine-4-carbonitrile,
1-(6-Chloro-7-[1,2,4]triazol-4-yl-3,4-dihydro-2H-benzo[1,4]oxazine-2-carbonyl)-4-(4-fluoro-benzyl)-piperidine-4-carbonitrile,
1-(6-Chloro-7-[1,2,3]triazol-1-yl-3,4-dihydro-2H-benzo[1,4]oxazine-2-carbonyl)-4-(4-fluoro-benzyl)-piperidine-4-carbonitrile,
1-(6-Chloro-4-methyl-7-oxazol-5-yl-3,4-dihydro-2H-benzo[1,4]oxazine-2-carbonyl)-4-(4-fluoro-benzyl)-piperidine-4-carbonitrile,
1-[6-Chloro-7-dimethylamino-4-(1H-[1,2,4]triazol-3-yl)-3,4-dihydro-2H-benzo[1,4]oxazine-2-carbonyl]-4-(4-fluoro-benzyl)-piperidine-4-carbonitrile,
1-[6-Chloro-7-(6-fluoro-pyridin-3-yl)-3,4-dihydro-2H-benzo[1,4]oxazine-2-carbonyl]-4-(4-fluoro-benzyl)-piperidine-4-carbonitrile,
1-[6-Chloro-7-(6-methoxy-pyridin-3-yl)-3,4-dihydro-2H-benzo[1,4]oxazine-2-carbonyl]-4-(4-fluoro-benzyl)-piperidine-4-carbonitrile,
1-[6-Chloro-7-(2-methoxy-pyridin-3-yl)-3,4-dihydro-2H-benzo[1,4]oxazine-2-carbonyl]-4-(4-fluoro-benzyl)-piperidine-4-carbonitrile,
1-(6-Chloro-7-hydroxymethyl-3,4-dihydro-2H-benzo[1,4]oxazine-2-carbonyl)-4-(4-fluoro-benzyl)-piperidine-4-carbonitrile,
1-(6-Chloro-2-methyl-3,4-dihydro-2H-benzo[1,4]oxazine-2-carbonyl)-4-(4-fluoro-benzyl)-piperidine-4-carbonitrile,
1-(6-Chloro-7-climethylamino-2-methyl-3,4-dihydro-2H-benzo[1,4]oxazine-2-carbonyl)-4-(4-fluoro-benzyl)-piperidine-4-carbonitrile,
6-Chloro-2-[4-cyano-4-(4-fluoro-benzyl)-piperidine-1-carbonyl]-7-dimethylamino-2,3-dihydro-benzo[1,4]oxazine-4-carbonitrile,
1-[6-Chloro-7-(2-methoxy-phenyl)-3,4-dihydro-2H-benzo[1,4]oxazine-2-carbonyl]-4-(4-fluoro-benzyl)-piperidine-4-carbonitrile,
1-(6-Chloro-7-pyridin-2-yl-3,4-dihydro-2H-benzo[1,4]oxazine-2-carbonyl)-4-(4-fluoro-benzyl)-piperidine-4-carbonitrile,
1-(7-Acetyl-6-chloro-3,4-dihydro-2H-benzo[1,4]oxazine-2-carbonyl)-4-(4-fluoro-benzyl)-piperidine-4-carbonitrile,
1-(6-Chloro-7-oxazol-5-yl-3,4-dihydro-2H-benzo[1,4]oxazine-2-carbonyl)-4-(4-fluoro-benzyl)-piperidine-4-carbonitrile,
1-(4-Chloro-2-methyl-3,6,7,8-tetrahydro-9-oxa-1,3,6-triaza-cyclopenta[a]naphthalene-8-carbonyl)-4-(4-fluoro-benzyl)-piperidine-4-carbonitrile,
1-(4-Chloro-3,6,7,8-tetrahydro-9-oxa-1,3,6triazacyclopenta[a]naphthalene-8-carbonyl)-4-(4-fluoro-benzyl)-piperidine-4-carbonitrile,
6-Chloro-2-[4-cyano-4-(4-fluoro-benzyl)-piperidine-1-carbonyl]-3,4-dihydro-2H-benzo[1,4]oxazine-7-carboxylic acid methylamide,
6-Chloro-2-[4-cyano-4-(4-fluoro-benzyl)-piperidine-1-carbonyl]-3,4-dihydro-2H-benzo[1,4]oxazine-7-carbonitrile,
6-Chloro-2-[4-cyano-4-(4-fluoro-benzyl)-piperidine-1-carbonyl]-3,4-dihydro-2H-benzo[1,4]oxazine-7-carboxylic acid dimethylamide, and
1-[6-Chloro-7-(2-methyl-thiazol-4-yl)-3,4-dihydro-2H-benzo[1,4]oxazine-2-carbonyl]-4-(4-fluoro-benzyl)-piperidine-4-carbonitrile,
and pharmaceutically acceptable salts thereof, solvates thereof, and solvates of pharmaceutically acceptable salts thereof,
wherein if the compound exhibits chirality it can be in the form of a mixture of enantiomers such as a racemate or a mixture of diastereomers, or can be in the form of a single enantiomer or a single diastereomer.

3. A compound selected from:
6-Chloro-3,4-dihydro-2H-benzo[1,4]oxazine-2-carboxylic acid [1-(4-fluoro-benzyl)-piperidin-4-yl]-amide,
N-[1-(4-Chloro-benzyl)-piperidin-4-yl]-2-(6-chloro-3,4-dihydro-2H-benzo[1,4]oxazin-2-yl)-acetamide,
[1-(4-Chloro-benzyl)-piperidin-4-yl]-[2-(6-chloro-3,4-dihydro-2H-benzo[1,4]oxazin-2-yl)-ethyl]-amine,
6-Chloro-7-methoxy-3,4-dihydro-2H-benzo[1,4]oxazine-2-carboxylic acid [1-(4-fluoro-benzyl)-piperidin-4-yl]-amide,
6-Chloro-7-methoxy-3,4-dihydro-2H-benzo[1,4]oxazine-2-carboxylic acid [1-(4-fluoro-benzyl)-4-hydroxy-piperidin-4-ylmethyl]-amide, and
6-Chloro-7-dimethylamino-3,4-dihydro-2H-benzo[1,4]oxazine-2-carboxylic acid [1-(4-fluoro-benzyl)-4-hydroxy-piperidin-4-ylmethyl]-amide,
and pharmaceutically acceptable salts thereof, solvates thereof, and solvates of pharmaceutically acceptable salts thereof,
wherein if the compound exhibits chirality it can be in the form of a mixture of enantiomers such as a racemate or a mixture of diastereomers, or can be in the form of a single enantiomer or a single diastereomer.

4. A pharmaceutical composition comprising a compound according to claim 1 and a pharmaceutical acceptable carrier.

5. A method for treating a condition that responds to a CC chemokine receptor ligand comprising administering to a patient in need thereof an effective amount of a composition according to claim 4.

6. The method of claim 5, wherein the CC chemokine receptor ligand is a CCR1 antagonist.

7. The method according to claim 5, wherein the condition is selected from a group consisting of inflammatory disorder, autoimmune diseases, diabetes, obesity, and metabolic syndrome.

8. The method according to claim 7, wherein the condition is inflammatory disorder.

9. The method according to claim 7, wherein the condition is autoimmune diseases.

10. The method according to claim 7, wherein the condition is diabetes.

11. The method according to claim 7, wherein the condition is obesity.
